(19) United States
(12) Patent Application Publication
(10) Pub. No.: US 2022/0173322 A1
(43) Pub. Date: Jun. 2, 2022

(54) ORGANIC ELECTROLUMINESCENT COMPOUND, A PLURALITY OF HOST MATERIALS COMPRISING THE SAME, AND ORGANIC ELECTROLUMINESCENT DEVICE

(71) Applicant: ROHM AND HAAS ELECTRONIC MATERIALS KOREA LTD., Chungcheongnam-do (KR)

(72) Inventors: Hyo-Soon PARK, Gyeonggi-do (KR); Young-Jae KIM, Gyeonggi-do (KR); Sang-Hee CHO, Gyeonggi-do (KR); Su-Hyun LEE, Gyeonggi-do (KR)

(21) Appl. No.: 17/528,997

(22) Filed: Nov. 17, 2021

(30) Foreign Application Priority Data
Dec. 1, 2020 (KR) 10-2020-0165390
Oct. 5, 2021 (KR) 10-2021-0131556

(51) Int. Cl. H01L 51/00

(52) U.S. Cl. CPC H01L 51/0055; H01L 51/0058; H01L 51/0067; H01L 51/0074; H01L 51/0073; H01L 51/5012; H01L 51/006; H01L 51/0061; H01L 51/0056; H01L 51/0054; H01L 2251/5384; H01L 51/0072

(57) ABSTRACT

The present disclosure relates to an organic electroluminescent compound, a plurality of host materials comprising the same, and an organic electroluminescent device. By comprising the organic electroluminescent compound and/or a plurality of host materials comprising the same according to the present disclosure, an organic electroluminescent device having low driving voltage and/or high luminous efficiency and/or long lifespan property can be provided.

### TECHNICAL FIELD

The present disclosure relates to an organic electroluminescent compound, a plurality of host materials comprising the same, and an organic electroluminescent device.

### BACKGROUND ART

An electroluminescent device (EL device) is a self-light-emitting display device which has advantages in that it provides a wider viewing angle, a greater contrast ratio, and a faster response time. An organic electroluminescent device (OLED) was first developed by Eastman Kodak in 1987, by using small aromatic diamine molecules and aluminum complexes as materials for forming a light-emitting layer [Appl. Phys. Lett. 51, 913, 1987].

The most important factor determining luminous efficiency in an OLED is the light-emitting materials used. The light-emitting material is classified into a host material and a dopant material in a functional aspect. A light-emitting material can be used as a combination of a host and a dopant to improve color purity, luminous efficiency, and stability. Generally, a device having excellent electroluminescent (EL) characteristics has a structure comprising a light-emitting layer formed by doping a dopant to a host. When using such a dopant/host material system as a light-emitting material, their selection is important since host materials greatly influence the efficiency and lifespan of the light-emitting device.

Recently, an urgent task is the development of an OLED having high efficiency and long lifespan property. In particular, the development of highly excellent light-emitting material over conventional light-emitting materials is urgently required, considering the EL properties necessary for medium and large-sized OLED panels.

Japanese Patent Publication No. JP 5185591 B2 discloses a specific dopant compound and a host compound having a polycyclic condensed aromatic skeleton such as benzochrysene. However, said reference does not specifically disclose an organic electroluminescent compound, and a specific combination of host materials comprising the same as described in the present disclosure. In addition, there is still a need for development of a light-emitting material having improved performances, such as improved driving voltage, luminous efficiency, and long lifespan property, compared to the conventional specific combination of compounds disclosed in said reference.

### DISCLOSURE OF THE INVENTION

### Problems to be Solved

The object of the present disclosure is firstly, to provide an organic electroluminescent compound which is able to produce an organic electroluminescent device having low driving voltage and/or high luminous efficiency and/or long lifespan property, and a plurality of host materials comprising the same, and secondly, to provide an organic electroluminescent device comprising the organic electroluminescent compound and/or the plurality of host materials.

### Solution to Problems

As a result of intensive studies to solve the technical problem above, the present inventors found that a compound having a benzo[c]chrysenyl moiety has an energy level suitable for use in an OLED, and particularly exhibits excellent device properties by combining with an aminoaryl or azine-based heteroaryl moiety, so that the present invention was completed. The organic electroluminescent compound according to the present disclosure exhibiting these characteristics is represented by the following formula 1.

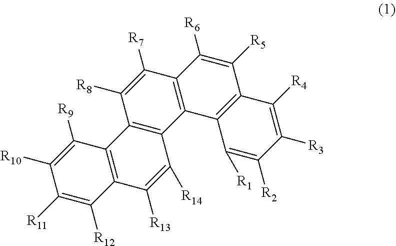

In formula 1,

R₁ to R₁₄ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of a (C3-C30) aliphatic ring and a (C6-C30) aromatic ring, -(L₁)ₙ-(HAr)ₘ, or -L₃-N—(Ar₃)(Ar₄); or may be linked to the adjacent substituent each other to form a ring(s);

provided that at least one of R₁ to R₁₄ is-(L₁)ₙ-(HAr)ₘ or -L₃-N—(Ar₃)(Ar₄);

L₁ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;

HAr represents a substituted or unsubstituted (C6-C30)aryl or a substituted or unsubstituted (3- to 30-membered)heteroaryl;

L₃ represents a single bond, a substituted or unsubstituted (C6-C30)arylene, or a substituted or unsubstituted (3- to 30-membered)heteroarylene;

Ar₃ and Ar₄ each independently represent hydrogen, a substituted or unsubstituted (C1-C30)alkyl, a substituted or unsubstituted (C2-C30)alkenyl, a substituted or unsubstituted fused ring of a (C3-C30) aliphatic ring and a (C6-C30) aromatic ring, a substituted or unsubstituted (C6-C30)aryl, or a substituted or unsubstituted (3- to 30-membered)heteroaryl; and

n and m each independently represent an integer of 1 or 2; and when n and m are an integer of 2, each of L₁ and each of HAr may be the same or different;

provided that the following compounds are excluded from the organic electroluminescent compound represented by the formula 1.

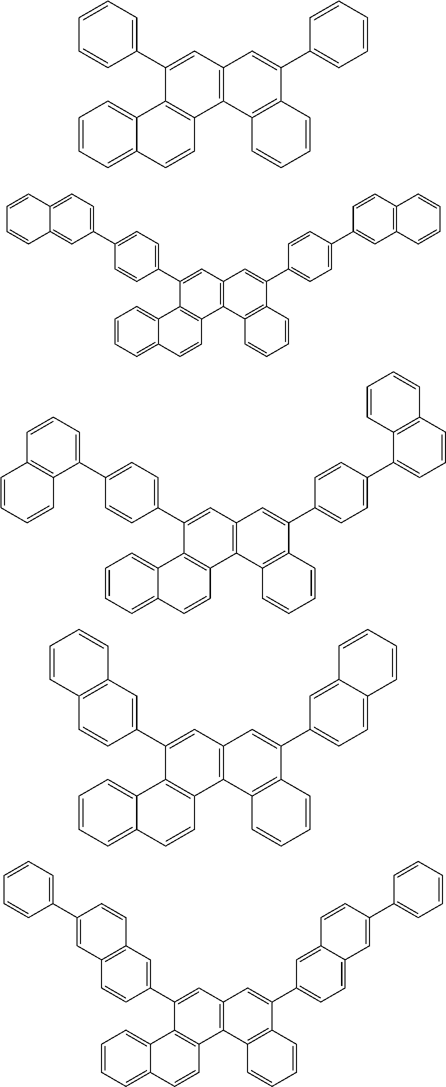

### Advantageous Effects of Invention

By comprising the organic electroluminescent compound according to the present disclosure, an organic electroluminescent device having low driving voltage and/or high luminous efficiency and/or long lifespan property can be provided.

### EMBODIMENTS OF THE INVENTION

Hereinafter, the present disclosure will be described in detail. However, the following description is intended to explain the invention, and is not meant in any way to restrict the scope of the invention.

The present disclosure relates to an organic electroluminescent compound represented by the formula 1, an organic electroluminescent material comprising the organic electroluminescent compound and an organic electroluminescent device comprising the organic electroluminescent compound.

The present disclosure relates to a plurality of host materials comprising a first host material including an organic electroluminescent compound represented by the formula 1 and a second host material, which is different from the first host material, and an organic electroluminescent device comprising the same.

The term “organic electroluminescent compound” in the present disclosure means a compound that may be used in an organic electroluminescent device, and may be comprised in any material layer constituting an organic electroluminescent device, as necessary.

Herein, “organic electroluminescent material” means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (containing host and dopant materials), an electron buffer material, a hole blocking material, an electron transport material, or an electron injection material, etc.

Herein, “a plurality of host materials” means an organic electroluminescent material comprising a combination of at least two host materials. It may mean both a material before being comprised in an organic electroluminescent device (e.g., before vapor deposition) and a material after being comprised in an organic electroluminescent device (e.g., after vapor deposition). A plurality of host materials of the present disclosure may be comprised in any light-emitting layer constituting an organic electroluminescent device. The at least two compounds comprised in a plurality of host materials may be comprised together in one light-emitting layer, or may each be comprised in separate light-emitting layers. When at least two host materials are comprised in one layer, the at least two host materials may be mixture-evaporated to form a layer or may be individually and simultaneously co-evaporated to form a layer.

Herein, “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. Herein, the term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. Herein, “(C6-C30)aryl(ene)” is a monocyclic or fused ring radical derived from an aromatic hydrocarbon having 6 to 30 ring backbone carbon atoms, in which the number of the ring backbone carbon atoms is preferably 6 to 20, more preferably 6 to 15, may be partially saturated, and may include a spiro structure. Examples of the aryl specifically may be phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluoren-fluoren]yl, spiro[fluoren-benzofluoren]yl, azulenyl, tetramethyl-dihydrophenanthrenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

Herein, “(3- to 30-membered)heteroaryl(ene)” is an aryl having 3 to 30 ring backbone atoms including at least one, preferably 1 to 4 heteroatoms selected from the group consisting of B, N, O, S. Si, P, Se, and Ge, in which the number of the ring backbone carbon atoms is preferably 5 to 24. The above heteroaryl(ene) may be a monocyclic ring, or a fused ring condensed with at least one benzene ring; and may be partially saturated. Also, the above heteroaryl or heteroarylene herein may be one formed by linking at least one heteroaryl or aryl group to a heteroaryl group via a single bond(s). Examples of the heteroaryl specifically may be a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthiridinyl, benzofuropyrimidinyl, naphthofuropyrimidinyl, benzothienoquinolinyl, benzothienoquinazolinyl, benzothienonaphthiridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolinyl, thiochromenoquinazolinyl, dimethylbenzopyrimidinyl, indolocarbazolyl, indenocarbazolyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthrdinyl, 10-phenanthridinyl, 1-acridinyl, 2-acrdinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Herein, the term “a fused ring of (C3-C30) aliphatic ring and (C6-C30) aromatic ring” means a ring formed by fusing at least one aliphatic ring having 3 to 30 ring backbone carbon atoms in which the carbon atoms number is preferably 3 to 25, more preferably 3 to 18, and at least one aromatic ring having 6 to 30 ring backbone carbon atoms in which the carbon atoms number is preferably 6 to 25, more preferably 6 to 18. For example, the fused ring may be a fused ring of at least one benzene and at least one cyclohexane, or a fused ring of at least one naphthalene and at least one cyclopentane, etc. Herein, the carbon atoms in the fused ring of (C3-C30) aliphatic ring and (C6-C30) aromatic ring may be replaced with at least one heteroatoms selected from B, N, O, S, Si and P, preferably at least one heteroatoms selected from N, O and S. The term “Halogen” in the present disclosure includes F, Cl, Br, and I.

In addition, “ortho (o).” “meta (m),” and “para (p)” are meant to signify the substitution position of all substituents. Ortho position is a compound with substituents, which are adjacent to each other, e.g., at the 1 and 2 positions on benzene. Meta position is the next substitution position of the immediately adjacent substitution position, e.g., a compound with substituents at the 1 and 3 positions on benzene. Para position is the next substitution position of the meta position, e.g., a compound with substituents at the 1 and 4 positions on benzene.

Herein, “a ring formed in linking to an adjacent substituent” means a substituted or unsubstituted (3- to 30-membered) mono- or polycyclic, alicyclic, aromatic ring, or a combination thereof, formed by linking or fusing two or more adjacent substituents, preferably may be a substituted or unsubstituted (3- to 26-membered) mono- or polycyclic, alicyclic, aromatic ring, or a combination thereof. Further, the formed ring may include at least one heteroatom selected from the group consisting of B, N, O, S, Si and P, preferably, N, O and S. According to one embodiment of the present disclosure, the number of atoms in the ring skeleton is 5 to 20; according to another embodiment of the present disclosure, the number of atoms in the ring skeleton is 5 to 15. In one embodiment, the fused ring may be, for example, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, a substituted or unsubstituted carbazole ring, etc.

In addition, “substituted” in the expression “substituted or unsubstituted” means that a hydrogen atom in a certain functional group is replaced with another atom or functional group, i.e., a substituent, and substituted with a group to which two or more substituents are connected among the substituents. For example, “a substituent to which two or more substituents are connected” may be pyridine-triazine. That is, pyridine-triazine may be heteroaryl or may be interpreted as one substituent in which two heteroaryls are connected. The substituents of the substituted (C1-C30)alkyl, the substituted (C2-C30)alkenyl, the substituted (C6-C30)aryl(ene), the substituted (3- to 30-membered)heteroaryl(ene), the substituted (C3-C30)cycloalkyl, the substituted (C1-C30)alkoxy, the substituted fused ring of (C3-C30) aliphatic ring and (C6-C30) aromatic ring, the substituted tri(C1-C30)alkylsilyl, the substituted di(C1-C30)alkyl(C6-C30)arylsilyl, the substituted (C1-C30)alkyldi(C6-C30)arylsilyl, and the substituted tri(C6-C30)arylsilyl in the formulas of the present disclosure, each independently represent, at least one selected from the group consisting of deuterium; halogen; cyano; carboxyl; nitro; hydroxy; (C1-C30)alkyl; halo(C1-C30)alkyl; (C2-C30)alkenyl; (C2-C30)alkynyl; (C1-C30)alkoxy; (C1-C30)alkylthio; (C3-C30)cycloalkyl; (C3-C30)cycloalkenyl; (3- to 7-membered)heterocycloalkyl; (C6-C30)aryloxy; (C6-C30)arylthio; (3- to 30-membered)heteroaryl unsubstituted or substituted with at least one (C6-C30)aryl; (C6-C30)aryl unsubstituted or substituted with at least one of (C1-C30)alkyl and (3- to 30-membered)heteroaryl; tri(C1-C30)alkylsilyl; tri(C6-C30)arylsilyl; di(C1-C30)alkyl(C6-C30)arylsilyl; (C1-C30)alkyldi(C6-C30)arylsilyl; amino; mono- or di-(C1-C30)alkylamino; mono- or di-(C2-C30)alkenylamino; mono- or di-(C6-C30)arylamino unsubstituted or substituted with (C1-C30)alkyl; mono- or di-(3- to 30-membered)heteroarylamino; (C1-C30)alkyl(C2-C30)alkenylamino; (C1-C30)alkyl(C6-C30)arylamino; (C1-C30)alkyl(3- to 30-membered)heteroarylamino; (C2-C30)alkenyl(C6-C30)arylamino; (C2-C30)alkenyl(3- to 30-membered)heteroarylamino; (C6-C30)aryl(3- to 30-membered)heteroarylamino; (C1-C30)alkylcarbonyl; (C1-C30)alkoxycarbonyl; (C6-C30)arylcarbonyl; (C6-C30)arylphosphinyl; di(C6-C30)arylboronyl; di(C1-C30)alkylboronyl; (C1-C30)alkyl(C6-C30)arylboronyl; (C6-C30)ar(C1-C30)alkyl; and (C1-C30)alkyl(C6-C30)aryl. For example, the substituents may be at least one selected from deuterium; methyl; tert-butyl; cyclohexyl; phenyl unsubstituted or substituted with at least one of methyl and tert-butyl; naphthyl; m-biphenyl; p-biphenyl; anthracenyl; fluoranthenyl; fluorenyl unsubstituted or substituted with (C1-C10)alkyl or (C6-C18)aryl; 9,10-dihydrophenanthrenyl unsubstituted or substituted with (C1-C10)alkyl; pyridyl unsubstituted or substituted with phenyl; phenoxazinyl; diphenylamino; benzimidazolyl substituted with phenyl; dibenzothiophenyl; and dibenzofuranyl.

Hereinafter, the organic electroluminescent compound according to one embodiment will be described.

The organic electroluminescent compound according to one embodiment is represented by the following formula 1.

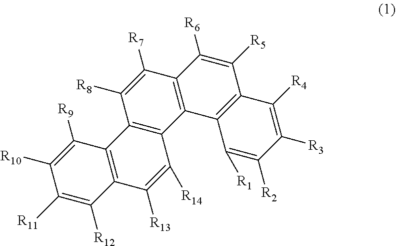

In formula 1,

R₁ to R₁₄ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of a (C3-C30) aliphatic ring and a (C6-C30) aromatic ring, -(L₁)ₙ-(HAr)ₘ, or -L₃-N—(Ar₃)(Ar₄); or the adjacent substituents may be linked to each other to form a ring(s);

provided that at least one of R₁ to R₁₄ is -(L₁)ₙ-(HAr)ₘ or -L₃-N—(Ar₃)(Ar₄);

L₁ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene:

HAr represents a substituted or unsubstituted (C6-C30)aryl or a substituted or unsubstituted (3- to 30-membered)heteroaryl;

L₃ represents a single bond, a substituted or unsubstituted (C6-C30)arylene, or a substituted or unsubstituted (3- to 30-membered)heteroarylene;

Ar₃ and Ar₄ each independently represent hydrogen, a substituted or unsubstituted (C1-C30)alkyl, a substituted or unsubstituted (C2-C30)alkenyl, a substituted or unsubstituted fused ring of a (C3-C30) aliphatic ring and a (C6-C30) aromatic ring, a substituted or unsubstituted (C6-C30)aryl, or a substituted or unsubstituted (3- to 30-membered)heteroaryl; and

n and m each independently represent an integer of 1 or 2; and when n and m are an integer of 2, each of L₁ and HAr may be the same or different;

provided that the following compounds are excluded from the organic electroluminescent compound represented by the formula 1.

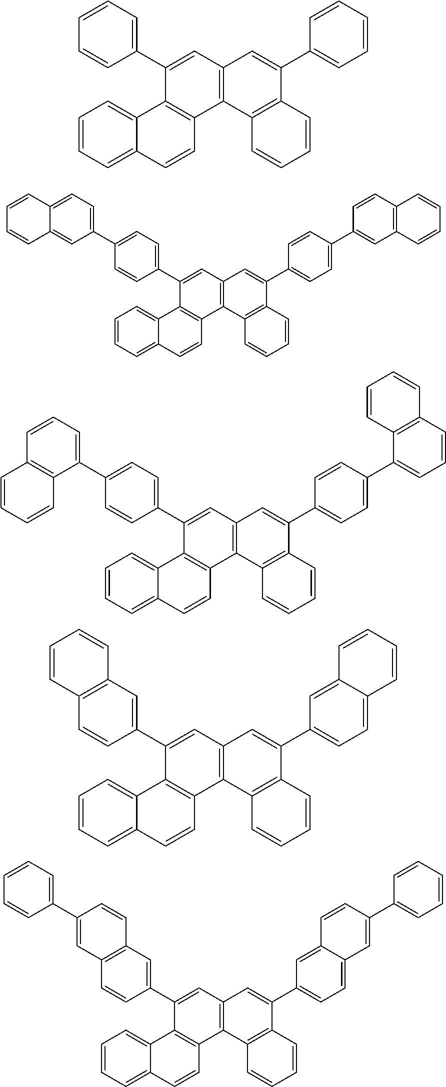

In one embodiment, R₁ to R₁₄ each independently may be hydrogen, deuterium, a substituted or unsubstituted (C6-C30)aryl, a substituted or unsubstituted (5- to 30-membered)heteroaryl, -(L₁)ₙ(HAr)ₘ, or -L₃-N—(Ar₃)(Ar₄), preferably hydrogen, deuterium, (C6-C25)aryl unsubstituted or substituted with a substituted or unsubstituted (5- to 30-membered)heteroaryl, a substituted or unsubstituted (5- to 25-membered)heteroaryl, -(L₁)ₙ-(HAr)ₘ, or -L₃-N—(Ar₃)(Ar₄), more preferably hydrogen, deuterium, (C6-C18)aryl unsubstituted or substituted with a substituted or unsubstituted (5- to 25-membered)heteroaryl, a substituted or unsubstituted (5- to 18-membered)heteroaryl, -(L₁)ₙ-(HAr)ₘ, or -L₃-N—(Ar₃)(Ar₄).

In one embodiment, at least one of R₁ to R₁₄ may be -(L₁)ₙ-(HAr)ₘ or -L₃-N—(Ar₃)(Ar₄).

In one embodiment, at least one of R₁ to R₁₄ may be -(L₁)ₙ-(HAr)ₘ.

In one embodiment, L₁ may be a single bond, a substituted or unsubstituted (C6-C30)arylene, or a substituted or unsubstituted (5- to 30-membered)heteroarylene, preferably a single bond, a substituted or unsubstituted (CM-C25)arylene, or a substituted or unsubstituted (5- to 25-membered)heteroarylene, more preferably a single bond, a substituted or unsubstituted (CM-C18)arylene, or a substituted or unsubstituted (5- to 18-membered)heteroarylene. For example, L₁ may be a single bond, a substituted or unsubstituted phenylene, a substituted or unsubstituted naphthylene, or a substituted or unsubstituted pyridylene.

In one embodiment, HAr may be a substituted or unsubstituted (C6-C30)aryl or a substituted or unsubstituted (5- to 30-membered)heteroaryl, preferably a substituted or unsubstituted (5- to 25-membered)heteroaryl, more preferably a substituted or unsubstituted (5- to 18-membered)heteroaryl comprising at least one nitrogen. For example, HAr may be a substituted or unsubstituted carbazolyl, a substituted or unsubstituted triazinyl, a substituted or unsubstituted quinoxalinyl, or a substituted or unsubstituted quinazolinyl. The substituents of the substituted carbazolyl, the substituted triazinyl, the substituted quinoxalinyl, and the substituted quinazolinyl may be at least one selected from a substituted or unsubstituted fused ring of a (C3-C30) aliphatic ring and a (C6-C30) aromatic ring; a substituted or unsubstituted (C6-C30)aryl; and a substituted or unsubstituted (5- to 30-membered)heteroaryl, preferably at least one selected from a substituted or unsubstituted fused ring of (C5-C25) aliphatic ring and (C6-C25) aromatic ring; a substituted or unsubstituted (C6-C25)aryl; and a substituted or unsubstituted (5- to 25-membered)heteroaryl. For example, the substituents may be at least one selected from phenyl; naphthyl; m-biphenyl; p-biphenyl; fluorenyl unsubstituted or substituted with (C1-C10)alkyl; 9,10-dihydrophenanthrenyl unsubstituted or substituted with (C1-C10)alkyl; pyridyl; dibenzothiophenyl; and dibenzofuranyl.

In one embodiment, at least one of R₁ to R₁₄ may be -L₃-N—(Ar₃)(Ar₄).

In one embodiment, L₃ may be a single bond.

In one embodiment, Ar₃ and Ar₄ each independently may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C18)aryl or a substituted or unsubstituted (5- to 18-membered)heteroaryl. For example, Ar₃ and Ar₄ each independently may be a substituted or unsubstituted phenyl, a substituted or unsubstituted naphthyl, a substituted or unsubstituted p-biphenyl, a substituted or unsubstituted m-biphenyl, a substituted or unsubstituted fluorenyl, a substituted or unsubstituted dibenzofuranyl, or a substituted or unsubstituted dibenzothiophenyl.

According to one embodiment, the organic electroluminescent compound represented by the formula 1 may be more specifically illustrated by the following compounds, but is not limited thereto.

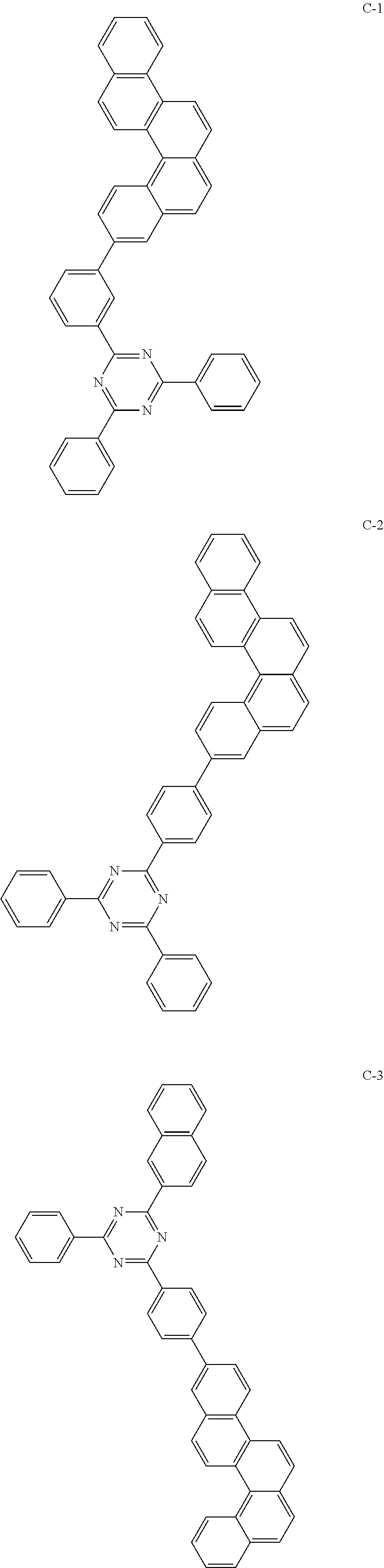 [SMILES: c1ccc(-c2nc(-c3ccccc3)nc(-c3cccc(-c4ccc5c(ccc6ccc7c8ccccc8ccc7c65)c4)c3)n2)cc1 ; c1ccc(-c2nc(-c3ccccc3)nc(-c3ccc(-c4ccc5c(ccc6ccc7c8ccccc8ccc7c65)c4)cc3)n2)cc1 ; c1ccc(-c2nc(-c3ccc(-c4ccc5c(ccc6c5ccc5ccc7ccccc7c56)c4)cc3)nc(-c3ccc4ccccc4c3)n2)cc1]

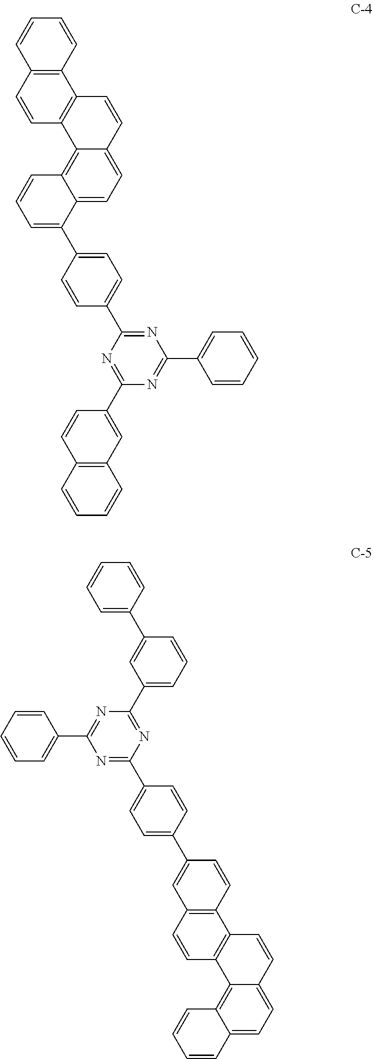 [SMILES: c1ccc(-c2nc(-c3ccc(-c4cccc5c4ccc4ccc6c7ccccc7ccc6c45)cc3)nc(-c3ccc4ccccc4c3)n2)cc1 ; c1ccc(-c2cccc(-c3nc(-c4ccccc4)nc(-c4ccc(-c5ccc6c(ccc7c6ccc6ccc8ccccc8c67)c5)cc4)n3)c2)cc1]

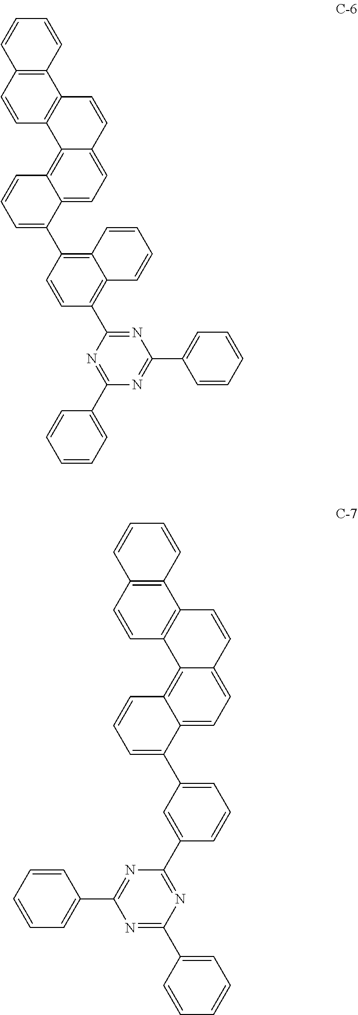 [SMILES: c1ccc(-c2nc(-c3ccccc3)nc(-c3ccc(-c4cccc5c4ccc4ccc6c7ccccc7ccc6c45)c4ccccc34)n2)cc1 ; c1ccc(-c2nc(-c3ccccc3)nc(-c3cccc(-c4cccc5c4ccc4ccc6c7ccccc7ccc6c45)c3)n2)cc1]

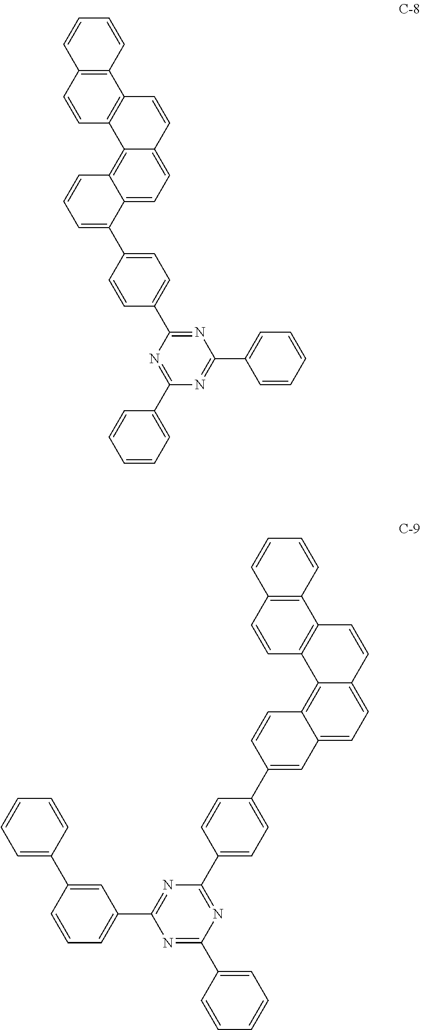 [SMILES: c1ccc(-c2nc(-c3ccccc3)nc(-c3ccc(-c4cccc5c4ccc4ccc6c7ccccc7ccc6c45)cc3)n2)cc1 ; c1ccc(-c2cccc(-c3nc(-c4ccccc4)nc(-c4ccc(-c5ccc6c(ccc7ccc8c9ccccc9ccc8c76)c5)cc4)n3)c2)cc1]

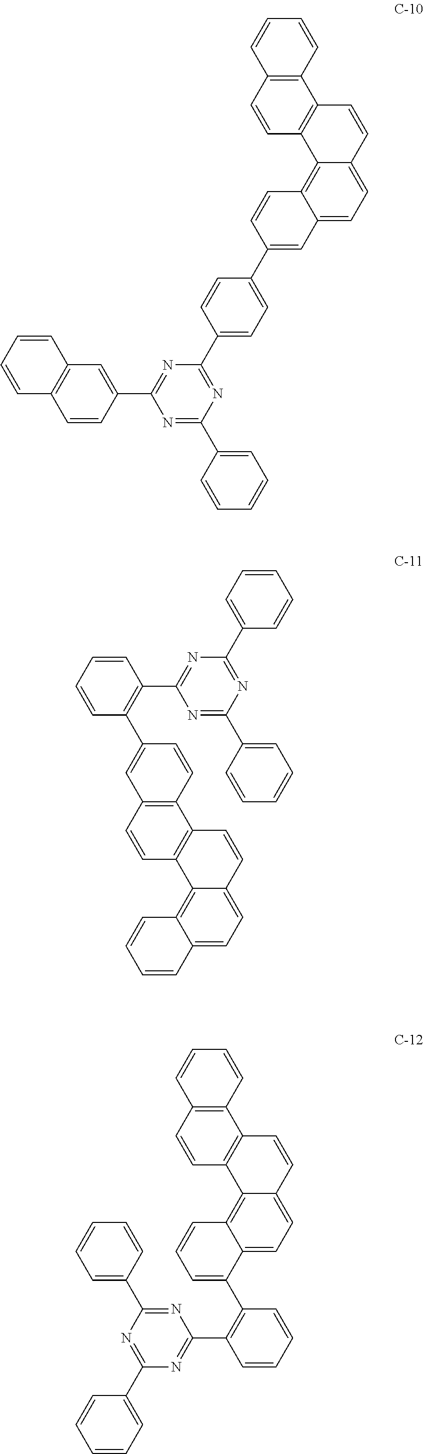 [SMILES: c1ccc(-c2nc(-c3ccc(-c4ccc5c(ccc6ccc7c8ccccc8ccc7c65)c4)cc3)nc(-c3ccc4ccccc4c3)n2)cc1 ; c1ccc(-c2nc(-c3ccccc3)nc(-c3ccccc3-c3ccc4c(ccc5c4ccc4ccc6ccccc6c45)c3)n2)cc1 ; c1ccc(-c2nc(-c3ccccc3)nc(-c3ccccc3-c3cccc4c3ccc3ccc5c6ccccc6ccc5c34)n2)cc1]

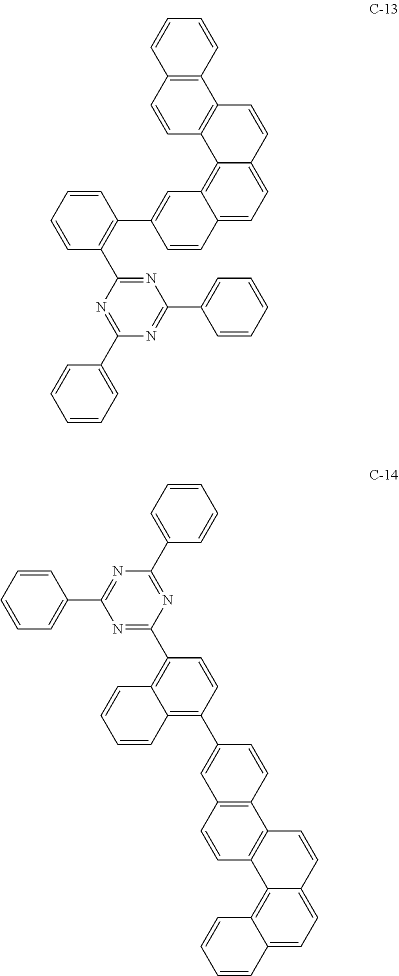 [SMILES: c1ccc(-c2nc(-c3ccccc3)nc(-c3ccccc3-c3ccc4ccc5ccc6c7ccccc7ccc6c5c4c3)n2)cc1 ; c1ccc(-c2nc(-c3ccccc3)nc(-c3ccc(-c4ccc5c(ccc6c5ccc5ccc7ccccc7c56)c4)c4ccccc34)n2)cc1]

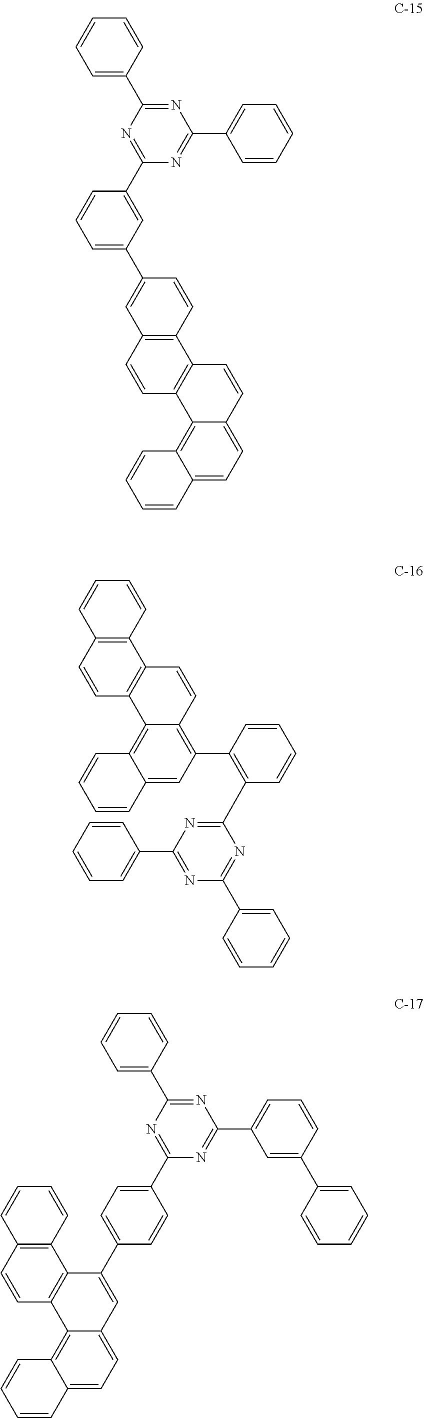 [SMILES: c1ccc(-c2nc(-c3ccccc3)nc(-c3cccc(-c4ccc5c(ccc6c5ccc5ccc7ccccc7c56)c4)c3)n2)cc1 ; c1ccc(-c2nc(-c3ccccc3)nc(-c3ccccc3-c3cc4ccccc4c4c3ccc3c5ccccc5ccc34)n2)cc1 ; c1ccc(-c2cccc(-c3nc(-c4ccccc4)nc(-c4ccc(-c5cc6ccc7ccccc7c6c6ccc7ccccc7c56)cc4)n3)c2)cc1]

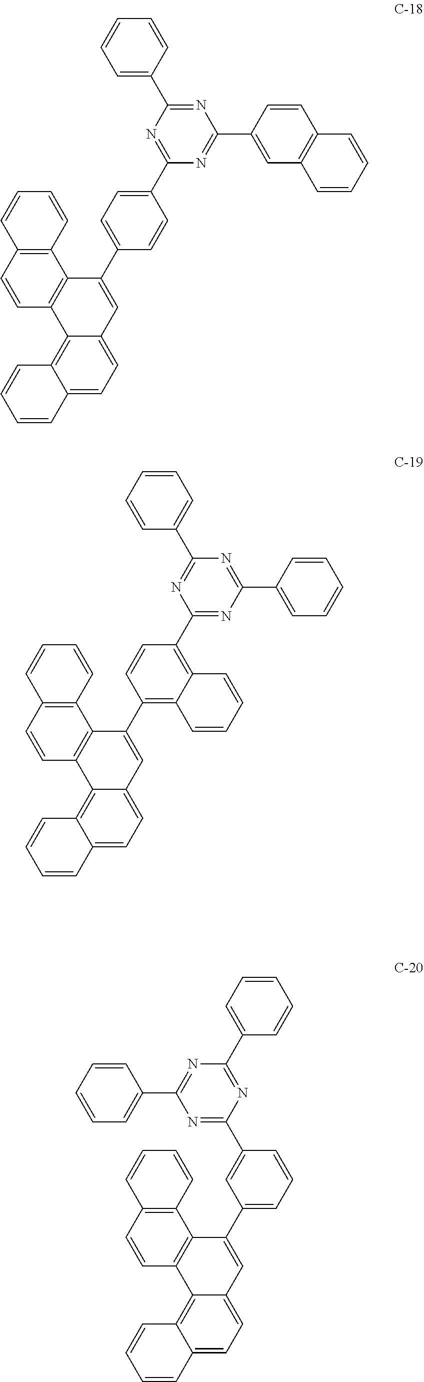 [SMILES: c1ccc(-c2nc(-c3ccc(-c4cc5ccc6ccccc6c5c5ccc6ccccc6c45)cc3)nc(-c3ccc4ccccc4c3)n2)cc1 ; c1ccc(-c2nc(-c3ccccc3)nc(-c3ccc(-c4cc5ccc6ccccc6c5c5ccc6ccccc6c45)c4ccccc34)n2)cc1 ; c1ccc(-c2nc(-c3ccccc3)nc(-c3cccc(-c4cc5ccc6ccccc6c5c5ccc6ccccc6c45)c3)n2)cc1]

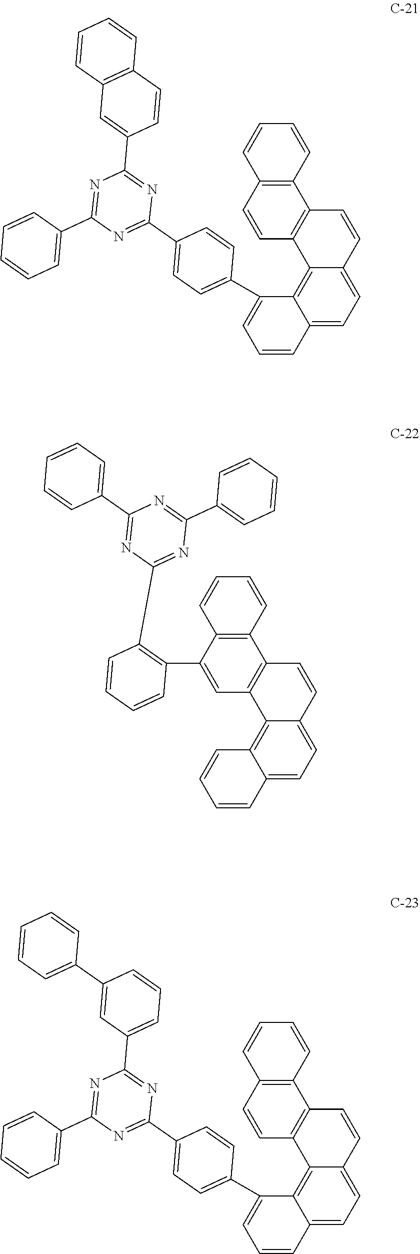 [SMILES: c1ccc(-c2nc(-c3ccc(-c4cccc5ccc6ccc7c8ccccc8ccc7c6c45)cc3)nc(-c3ccc4ccccc4c3)n2)cc1 ; c1ccc(-c2nc(-c3ccccc3)nc(-c3ccccc3-c3cc4c(ccc5ccc6ccccc6c54)c4ccccc34)n2)cc1 ; c1ccc(-c2cccc(-c3nc(-c4ccccc4)nc(-c4ccc(-c5cccc6ccc7ccc8c9ccccc9ccc8c7c56)cc4)n3)c2)cc1]

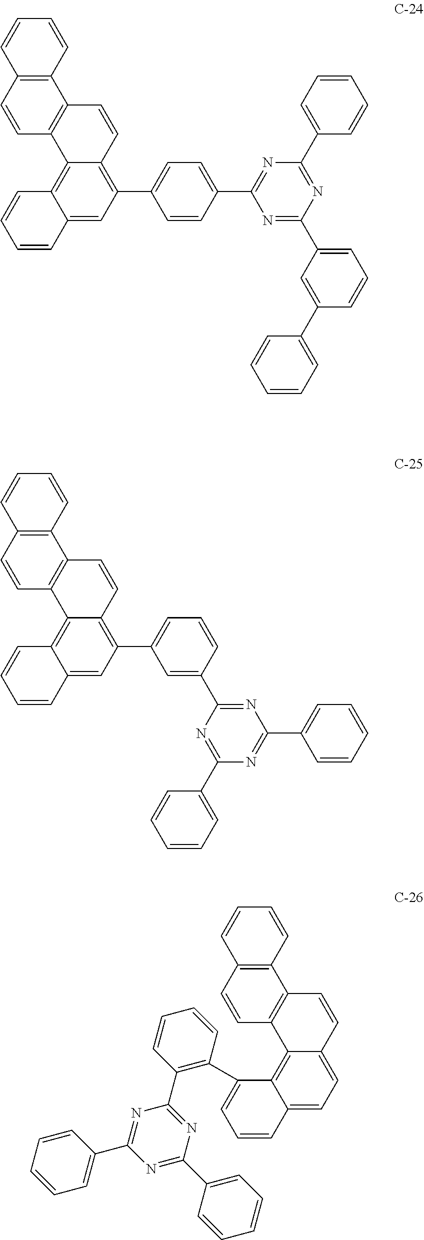 [SMILES: c1ccc(-c2cccc(-c3nc(-c4ccccc4)nc(-c4ccc(-c5cc6ccccc6c6c5ccc5c7ccccc7ccc56)cc4)n3)c2)cc1 ; c1ccc(-c2nc(-c3ccccc3)nc(-c3cccc(-c4cc5ccccc5c5c4ccc4c6ccccc6ccc45)c3)n2)cc1 ; c1ccc(-c2nc(-c3ccccc3)nc(-c3ccccc3-c3cccc4ccc5ccc6c7ccccc7ccc6c5c34)n2)cc1]

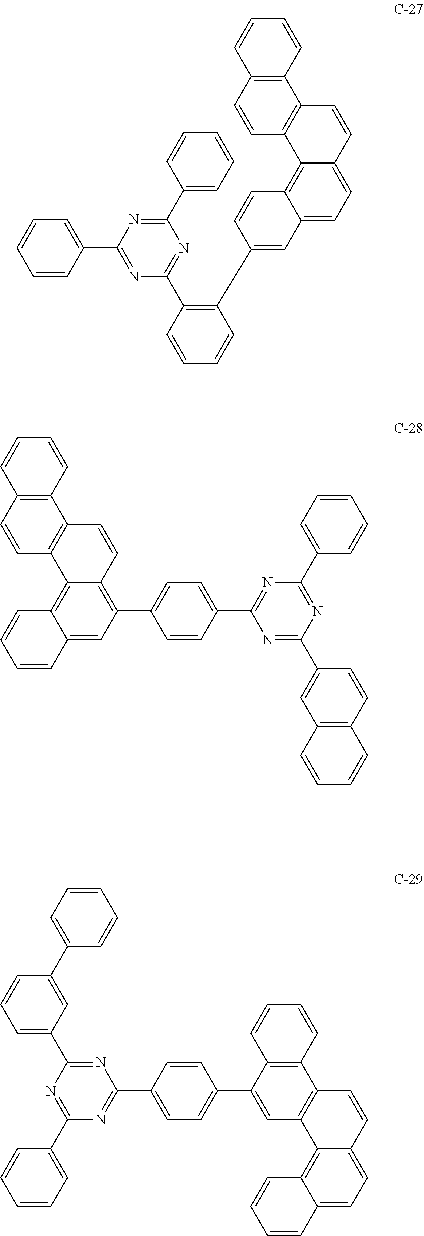 [SMILES: c1ccc(-c2nc(-c3ccccc3)nc(-c3ccccc3-c3ccc4c(ccc5ccc6c7ccccc7ccc6c54)c3)n2)cc1 ; c1ccc(-c2nc(-c3ccc(-c4cc5ccccc5c5c4ccc4c6ccccc6ccc45)cc3)nc(-c3ccc4ccccc4c3)n2)cc1 ; c1ccc(-c2cccc(-c3nc(-c4ccccc4)nc(-c4ccc(-c5cc6c(ccc7ccc8ccccc8c76)c6ccccc56)cc4)n3)c2)cc1]

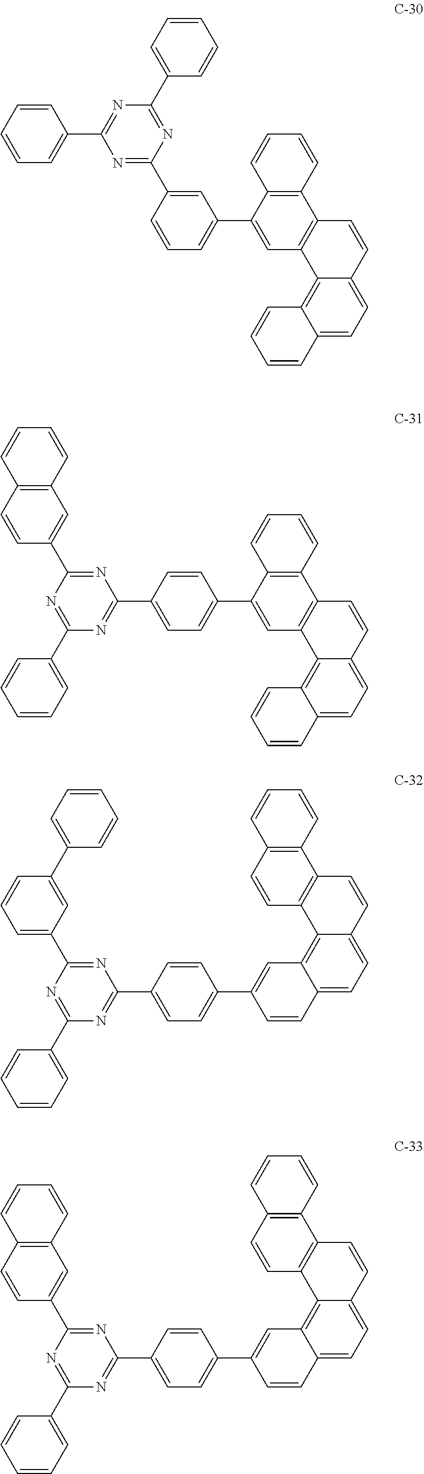

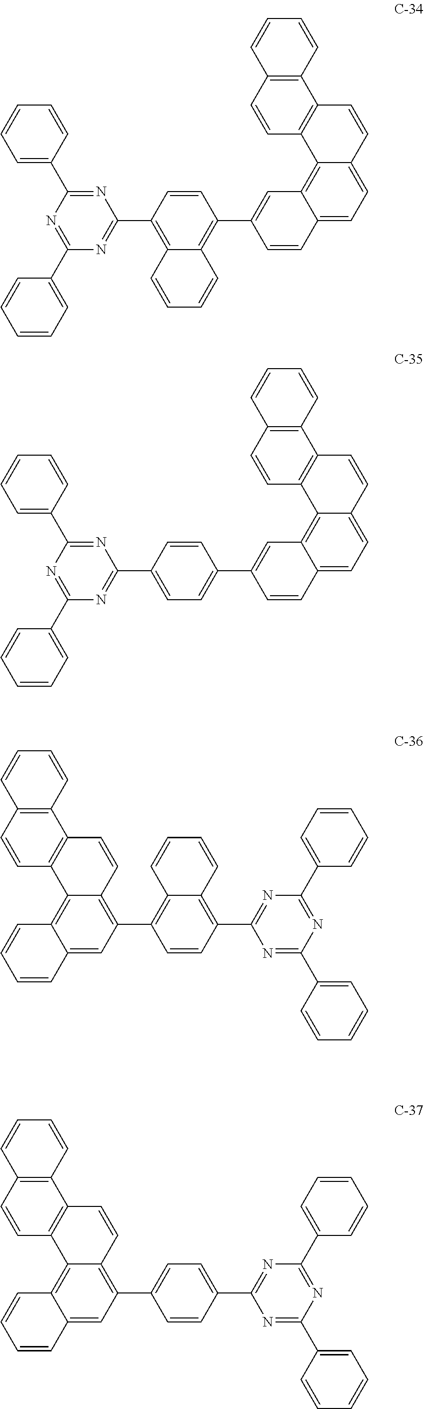 [SMILES: c1ccc(-c2nc(-c3ccccc3)nc(-c3ccc(-c4ccc5ccc6ccc7c8ccccc8ccc7c6c5c4)c4ccccc34)n2)cc1 ; c1ccc(-c2nc(-c3ccccc3)nc(-c3ccc(-c4ccc5ccc6ccc7c8ccccc8ccc7c6c5c4)cc3)n2)cc1 ; c1ccc(-c2nc(-c3ccccc3)nc(-c3ccc(-c4cc5ccccc5c5c4ccc4c6ccccc6ccc45)c4ccccc34)n2)cc1 ; c1ccc(-c2nc(-c3ccccc3)nc(-c3ccc(-c4cc5ccccc5c5c4ccc4c6ccccc6ccc45)cc3)n2)cc1]

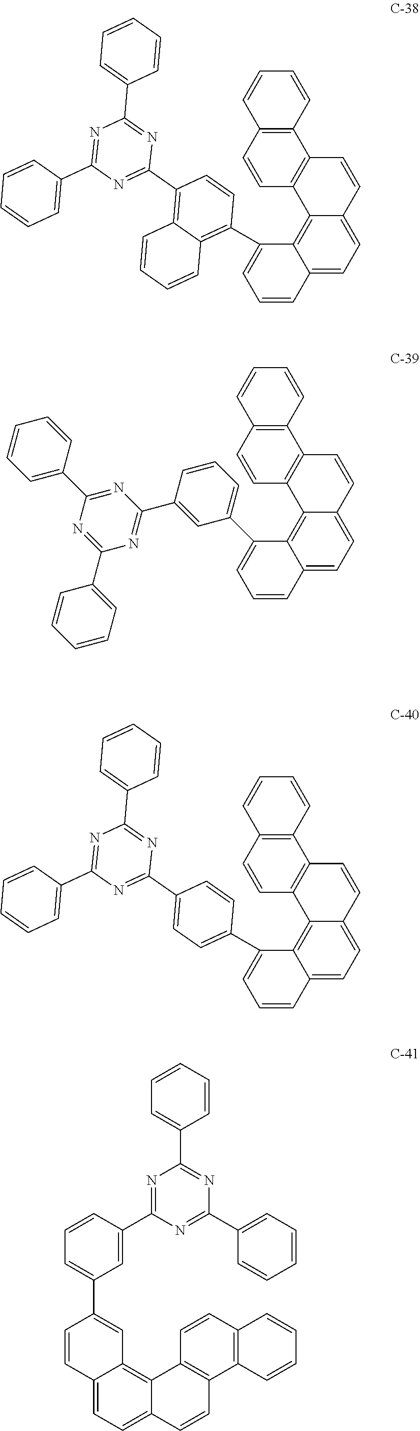 [SMILES: c1ccc(-c2nc(-c3ccccc3)nc(-c3ccc(-c4cccc5ccc6ccc7c8ccccc8ccc7c6c45)c4ccccc34)n2)cc1 ; c1ccc(-c2nc(-c3ccccc3)nc(-c3cccc(-c4cccc5ccc6ccc7c8ccccc8ccc7c6c45)c3)n2)cc1 ; c1ccc(-c2nc(-c3ccccc3)nc(-c3ccc(-c4cccc5ccc6ccc7c8ccccc8ccc7c6c45)cc3)n2)cc1 ; c1ccc(-c2nc(-c3ccccc3)nc(-c3cccc(-c4ccc5ccc6ccc7c8ccccc8ccc7c6c5c4)c3)n2)cc1]

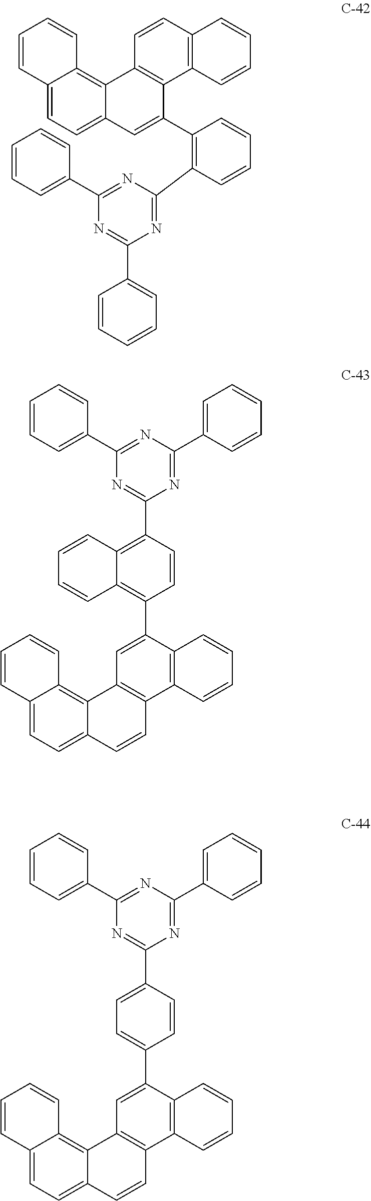 [SMILES: c1ccc(-c2nc(-c3ccccc3)nc(-c3ccccc3-c3cc4ccc5ccccc5c4c4ccc5ccccc5c34)n2)cc1 ; c1ccc(-c2nc(-c3ccccc3)nc(-c3ccc(-c4cc5c(ccc6ccc7ccccc7c65)c5ccccc45)c4ccccc34)n2)cc1 ; c1ccc(-c2nc(-c3ccccc3)nc(-c3ccc(-c4cc5c(ccc6ccc7ccccc7c65)c5ccccc45)cc3)n2)cc1]

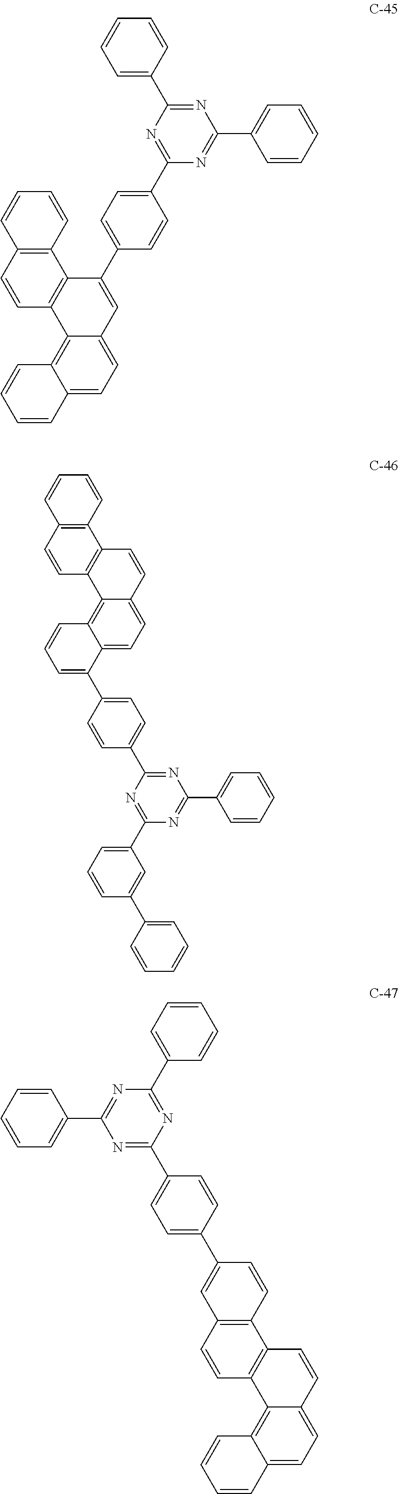

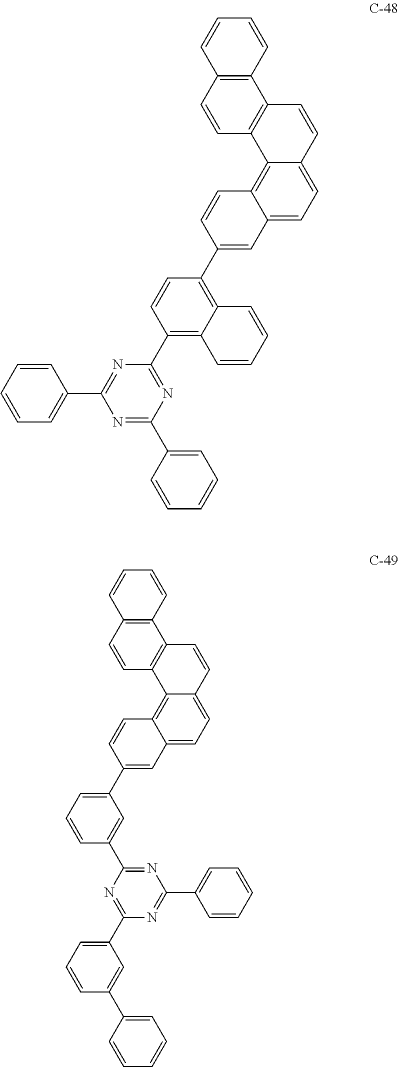 [SMILES: c1ccc(-c2nc(-c3ccccc3)nc(-c3ccc(-c4ccc5c(ccc6ccc7c8ccccc8ccc7c65)c4)c4ccccc34)n2)cc1 ; c1ccc(-c2cccc(-c3nc(-c4ccccc4)nc(-c4cccc(-c5ccc6c(ccc7ccc8c9ccccc9ccc8c76)c5)c4)n3)c2)cc1]

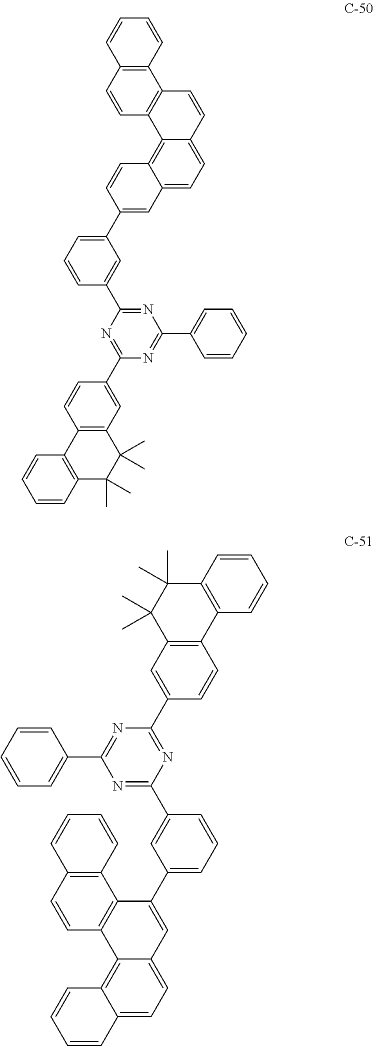 [SMILES: CC1(C)c2ccccc2-c2ccc(-c3nc(-c4ccccc4)nc(-c4cccc(-c5ccc6c(ccc7ccc8c9ccccc9ccc8c76)c5)c4)n3)cc2C1(C)C ; CC1(C)c2ccccc2-c2ccc(-c3nc(-c4ccccc4)nc(-c4cccc(-c5cc6ccc7ccccc7c6c6ccc7ccccc7c56)c4)n3)cc2C1(C)C]

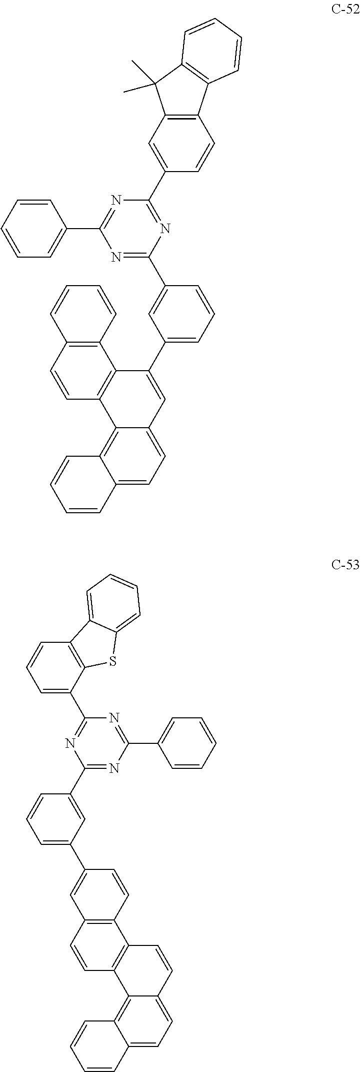 [SMILES: CC1(C)c2ccccc2-c2ccc(-c3nc(-c4ccccc4)nc(-c4cccc(-c5cc6ccc7ccccc7c6c6ccc7ccccc7c56)c4)n3)cc21 ; c1ccc(-c2nc(-c3cccc(-c4ccc5c(ccc6c5ccc5ccc7ccccc7c56)c4)c3)nc(-c3cccc4c3sc3ccccc34)n2)cc1]

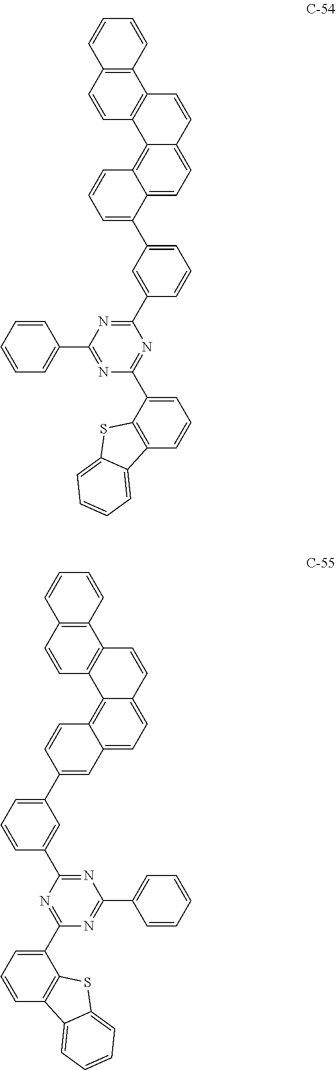 [SMILES: c1ccc(-c2nc(-c3cccc(-c4cccc5c4ccc4ccc6c7ccccc7ccc6c45)c3)nc(-c3cccc4c3sc3ccccc34)n2)cc1 ; c1ccc(-c2nc(-c3cccc(-c4ccc5c(ccc6ccc7c8ccccc8ccc7c65)c4)c3)nc(-c3cccc4c3sc3ccccc34)n2)cc1]

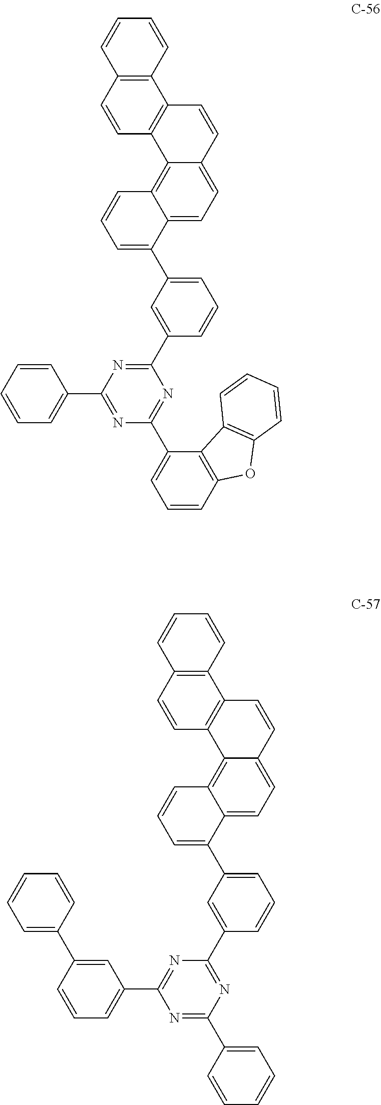 [SMILES: c1ccc(-c2nc(-c3cccc(-c4cccc5c4ccc4ccc6c7ccccc7ccc6c45)c3)nc(-c3cccc4oc5ccccc5c34)n2)cc1 ; c1ccc(-c2cccc(-c3nc(-c4ccccc4)nc(-c4cccc(-c5cccc6c5ccc5ccc7c8ccccc8ccc7c56)c4)n3)c2)cc1]

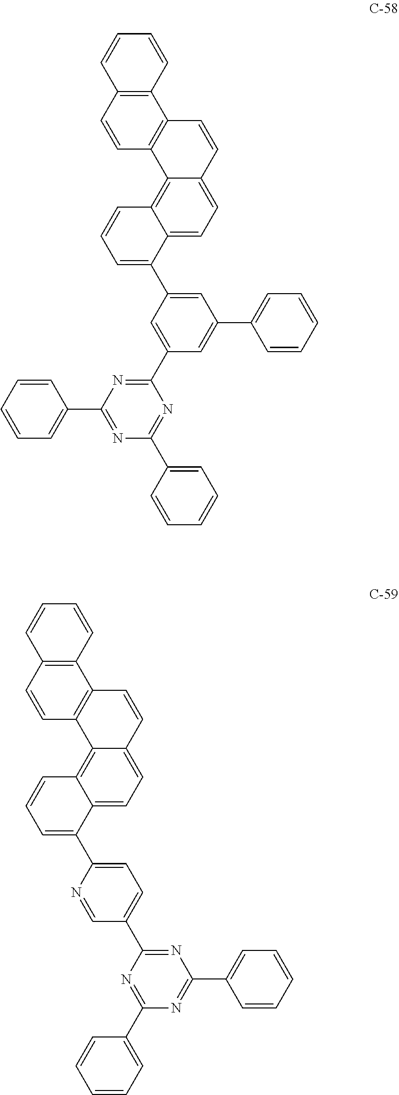 [SMILES: c1ccc(-c2cc(-c3nc(-c4ccccc4)nc(-c4ccccc4)n3)cc(-c3cccc4c3ccc3ccc5c6ccccc6ccc5c34)c2)cc1 ; c1ccc(-c2nc(-c3ccccc3)nc(-c3ccc(-c4cccc5c4ccc4ccc6c7ccccc7ccc6c45)nc3)n2)cc1]

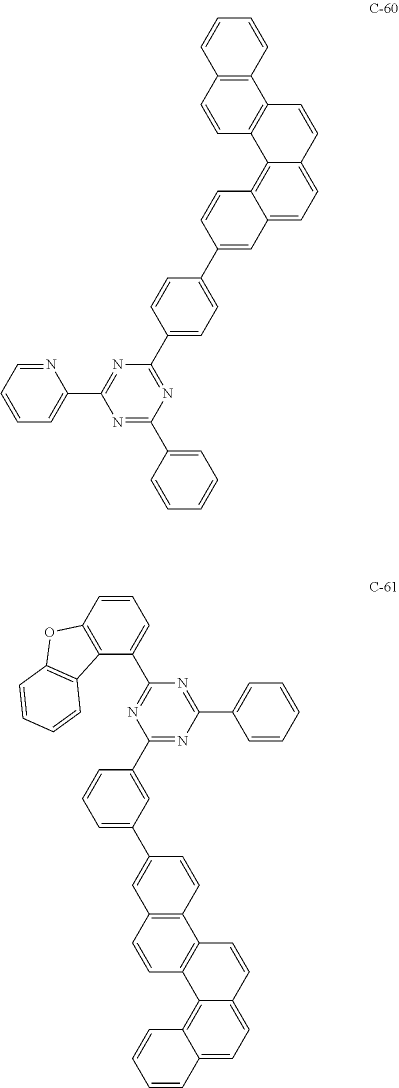 [SMILES: c1ccc(-c2nc(-c3ccc(-c4ccc5c(ccc6ccc7c8ccccc8ccc7c65)c4)cc3)nc(-c3ccccn3)n2)cc1 ; c1ccc(-c2nc(-c3cccc(-c4ccc5c(ccc6c5ccc5ccc7ccccc7c56)c4)c3)nc(-c3cccc4oc5ccccc5c34)n2)cc1]

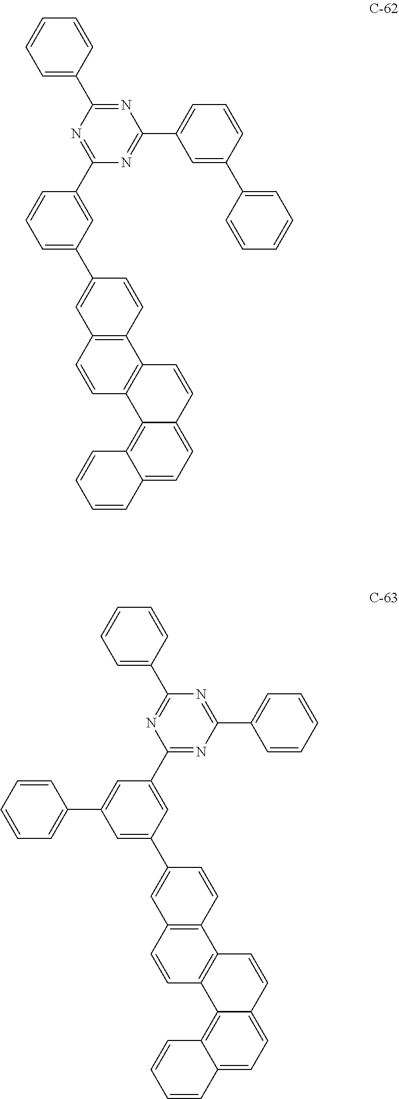 [SMILES: c1ccc(-c2cccc(-c3nc(-c4ccccc4)nc(-c4cccc(-c5ccc6c(ccc7c6ccc6ccc8ccccc8c67)c5)c4)n3)c2)cc1 ; c1ccc(-c2cc(-c3ccc4c(ccc5c4ccc4ccc6ccccc6c45)c3)cc(-c3nc(-c4ccccc4)nc(-c4ccccc4)n3)c2)cc1]

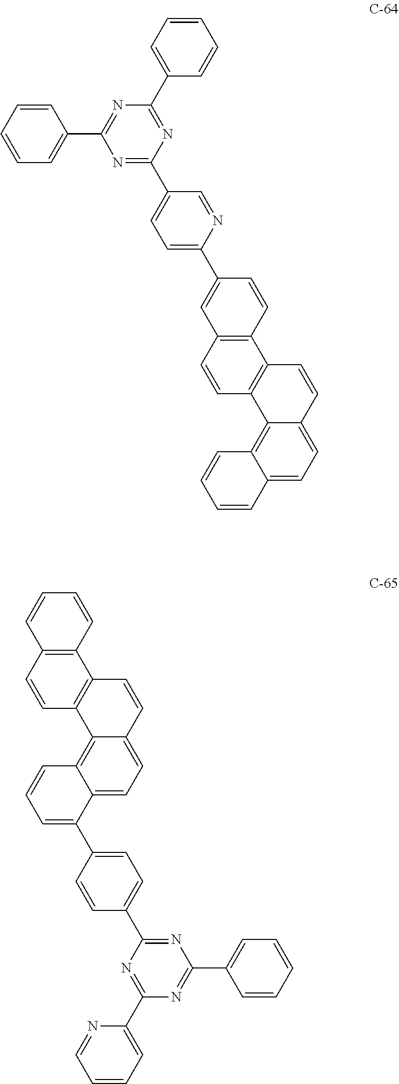 [SMILES: c1ccc(-c2nc(-c3ccccc3)nc(-c3ccc(-c4ccc5c(ccc6c5ccc5ccc7ccccc7c56)c4)nc3)n2)cc1 ; c1ccc(-c2nc(-c3ccc(-c4cccc5c4ccc4ccc6c7ccccc7ccc6c45)cc3)nc(-c3ccccn3)n2)cc1]

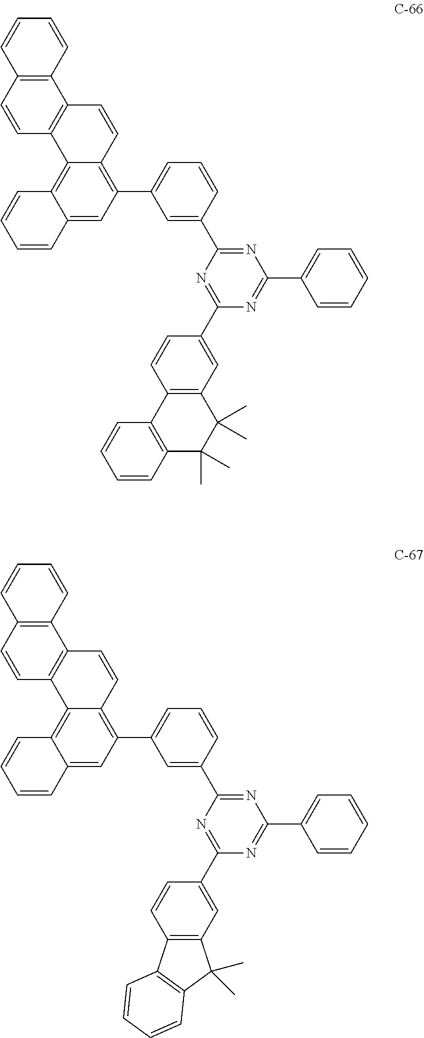 [SMILES: CC1(C)c2ccccc2-c2ccc(-c3nc(-c4ccccc4)nc(-c4cccc(-c5cc6ccccc6c6c5ccc5c7ccccc7ccc56)c4)n3)cc2C1(C)C ; CC1(C)c2ccccc2-c2ccc(-c3nc(-c4ccccc4)nc(-c4cccc(-c5cc6ccccc6c6c5ccc5c7ccccc7ccc56)c4)n3)cc21]

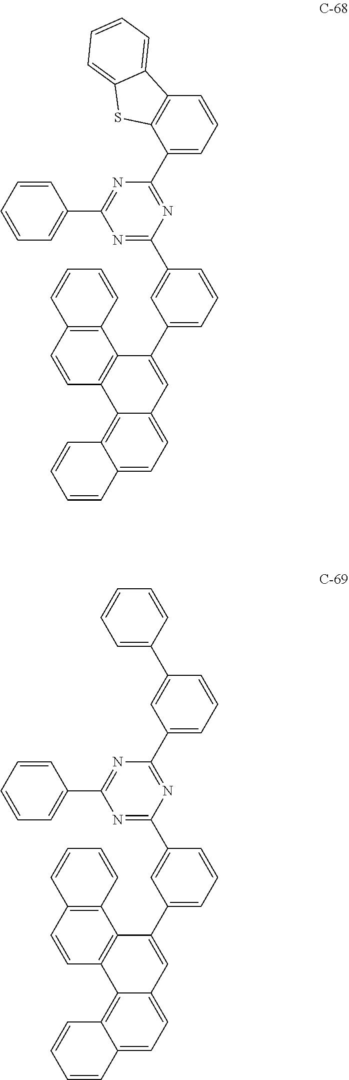 [SMILES: c1ccc(-c2nc(-c3cccc(-c4cc5ccc6ccccc6c5c5ccc6ccccc6c45)c3)nc(-c3cccc4c3sc3ccccc34)n2)cc1 ; c1ccc(-c2cccc(-c3nc(-c4ccccc4)nc(-c4cccc(-c5cc6ccc7ccccc7c6c6ccc7ccccc7c56)c4)n3)c2)cc1]

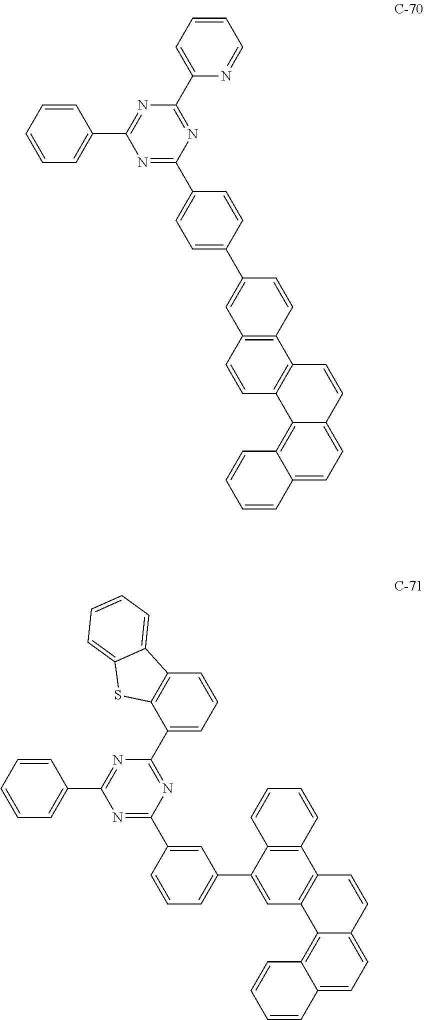 [SMILES: c1ccc(-c2nc(-c3ccc(-c4ccc5c(ccc6c5ccc5ccc7ccccc7c56)c4)cc3)nc(-c3ccccn3)n2)cc1 ; c1ccc(-c2nc(-c3cccc(-c4cc5c(ccc6ccc7ccccc7c65)c5ccccc45)c3)nc(-c3cccc4c3sc3ccccc34)n2)cc1]

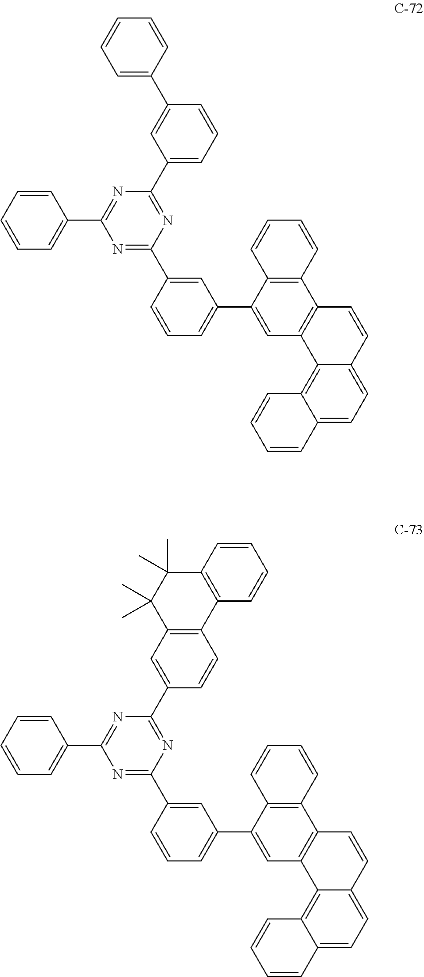 [SMILES: c1ccc(-c2cccc(-c3nc(-c4ccccc4)nc(-c4cccc(-c5cc6c(ccc7ccc8ccccc8c76)c6ccccc56)c4)n3)c2)cc1 ; CC1(C)c2ccccc2-c2ccc(-c3nc(-c4ccccc4)nc(-c4cccc(-c5cc6c(ccc7ccc8ccccc8c76)c6ccccc56)c4)n3)cc2C1(C)C]

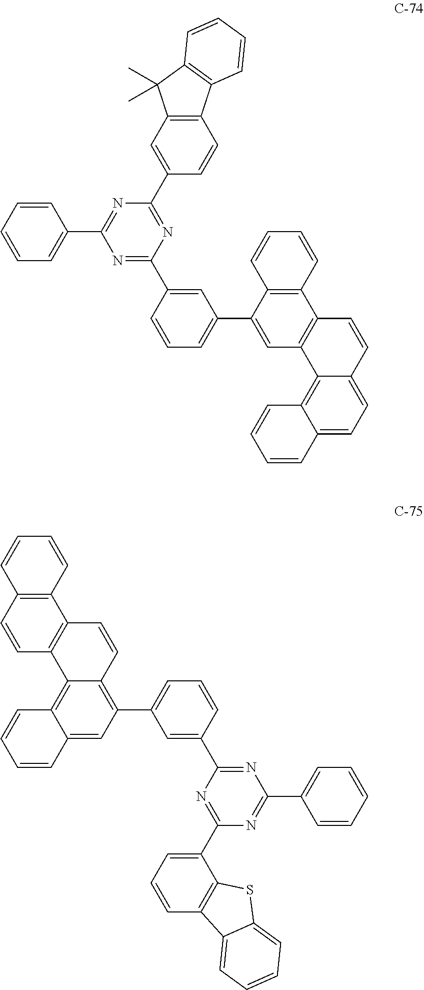 [SMILES: CC1(C)c2ccccc2-c2ccc(-c3nc(-c4ccccc4)nc(-c4cccc(-c5cc6c(ccc7ccc8ccccc8c76)c6ccccc56)c4)n3)cc21 ; c1ccc(-c2nc(-c3cccc(-c4cc5ccccc5c5c4ccc4c6ccccc6ccc45)c3)nc(-c3cccc4c3sc3ccccc34)n2)cc1]

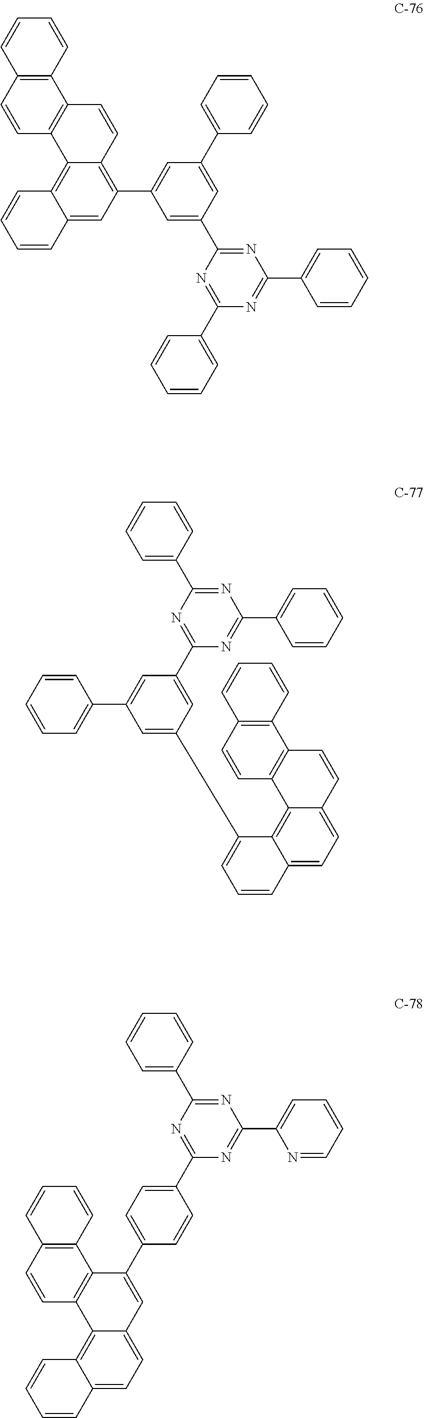 [SMILES: c1ccc(-c2cc(-c3nc(-c4ccccc4)nc(-c4ccccc4)n3)cc(-c3cc4ccccc4c4c3ccc3c5ccccc5ccc34)c2)cc1 ; c1ccc(-c2cc(-c3nc(-c4ccccc4)nc(-c4ccccc4)n3)cc(-c3cccc4ccc5ccc6c7ccccc7ccc6c5c34)c2)cc1 ; c1ccc(-c2nc(-c3ccc(-c4cc5ccc6ccccc6c5c5ccc6ccccc6c45)cc3)nc(-c3ccccn3)n2)cc1]

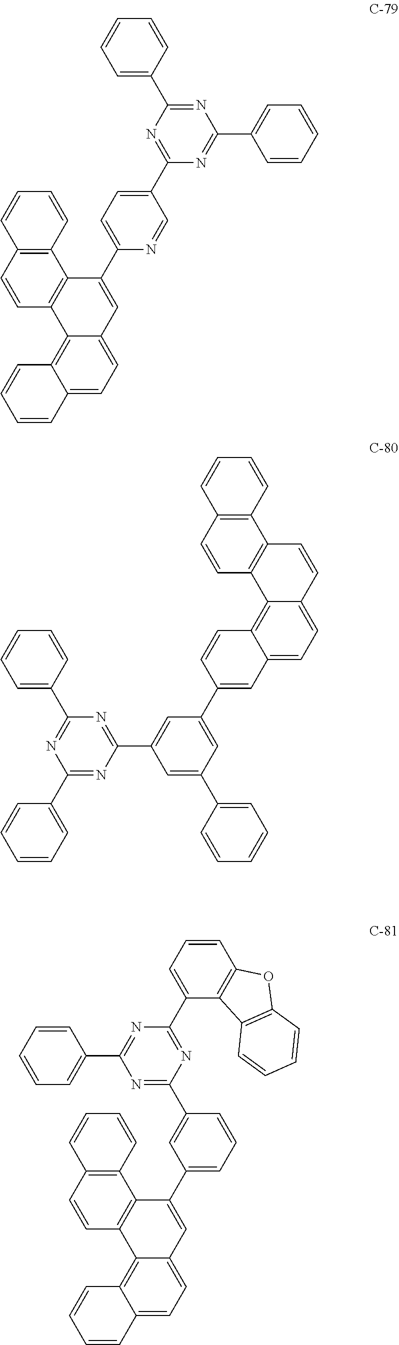 [SMILES: c1ccc(-c2nc(-c3ccccc3)nc(-c3ccc(-c4cc5ccc6ccccc6c5c5ccc6ccccc6c45)nc3)n2)cc1 ; c1ccc(-c2cc(-c3ccc4c(ccc5ccc6c7ccccc7ccc6c54)c3)cc(-c3nc(-c4ccccc4)nc(-c4ccccc4)n3)c2)cc1 ; c1ccc(-c2nc(-c3cccc(-c4cc5ccc6ccccc6c5c5ccc6ccccc6c45)c3)nc(-c3cccc4oc5ccccc5c34)n2)cc1]

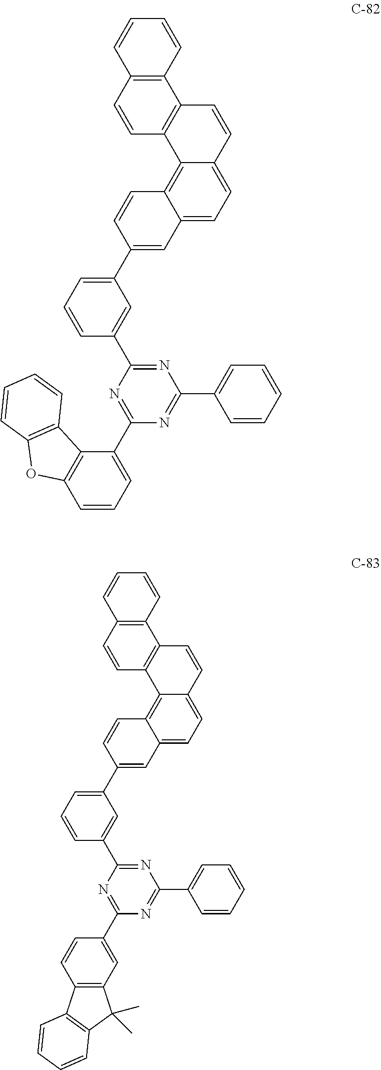 [SMILES: c1ccc(-c2nc(-c3cccc(-c4ccc5c(ccc6ccc7c8ccccc8ccc7c65)c4)c3)nc(-c3cccc4oc5ccccc5c34)n2)cc1 ; CC1(C)c2ccccc2-c2ccc(-c3nc(-c4ccccc4)nc(-c4cccc(-c5ccc6c(ccc7ccc8c9ccccc9ccc8c76)c5)c4)n3)cc21]

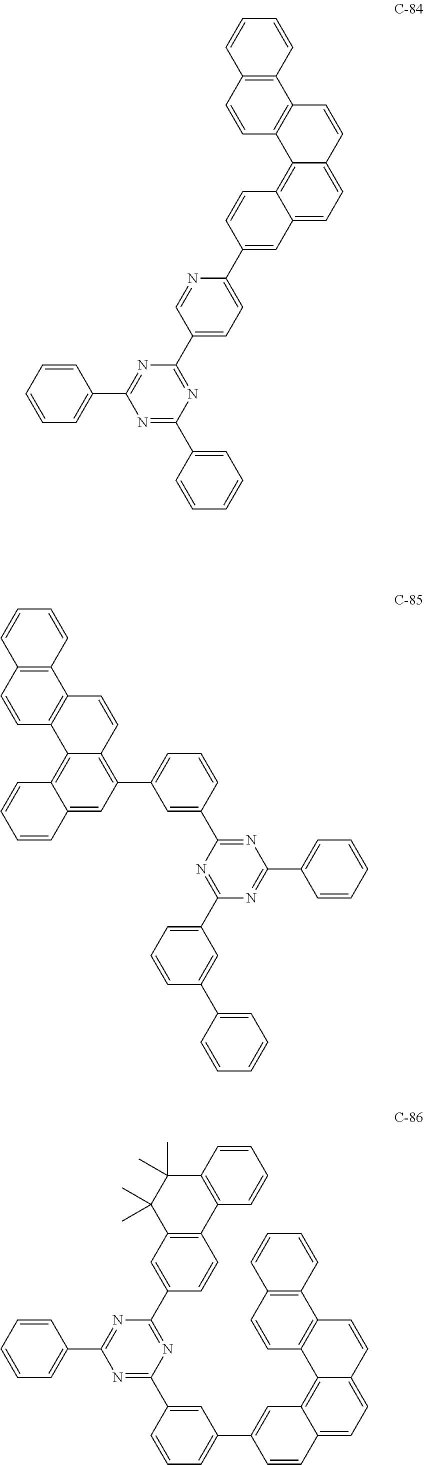 [SMILES: c1ccc(-c2nc(-c3ccccc3)nc(-c3ccc(-c4ccc5c(ccc6ccc7c8ccccc8ccc7c65)c4)nc3)n2)cc1 ; c1ccc(-c2cccc(-c3nc(-c4ccccc4)nc(-c4cccc(-c5cc6ccccc6c6c5ccc5c7ccccc7ccc56)c4)n3)c2)cc1 ; CC1(C)c2ccccc2-c2ccc(-c3nc(-c4ccccc4)nc(-c4cccc(-c5ccc6ccc7ccc8c9ccccc9ccc8c7c6c5)c4)n3)cc2C1(C)C]

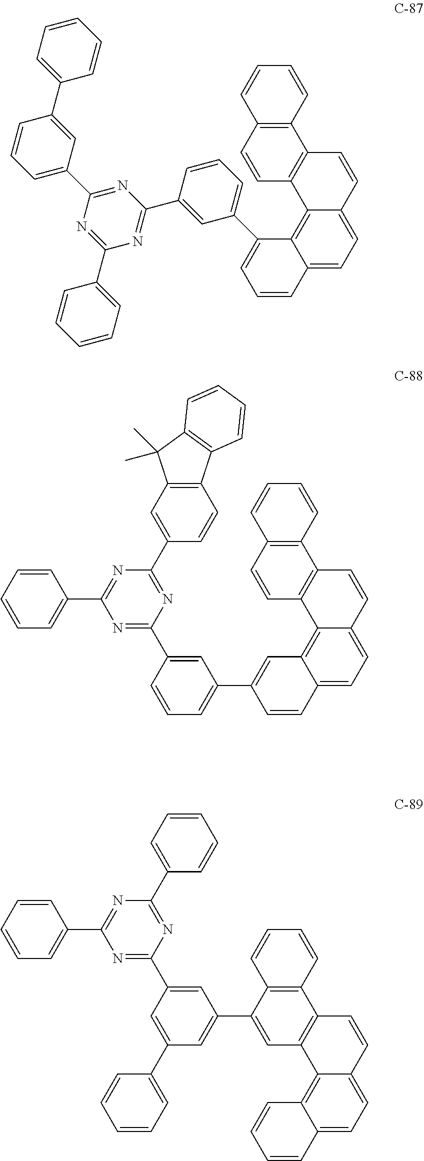 [SMILES: c1ccc(-c2cccc(-c3nc(-c4ccccc4)nc(-c4cccc(-c5cccc6ccc7ccc8c9ccccc9ccc8c7c56)c4)n3)c2)cc1 ; CC1(C)c2ccccc2-c2ccc(-c3nc(-c4ccccc4)nc(-c4cccc(-c5ccc6ccc7ccc8c9ccccc9ccc8c7c6c5)c4)n3)cc21 ; c1ccc(-c2cc(-c3nc(-c4ccccc4)nc(-c4ccccc4)n3)cc(-c3cc4c(ccc5ccc6ccccc6c54)c4ccccc34)c2)cc1]

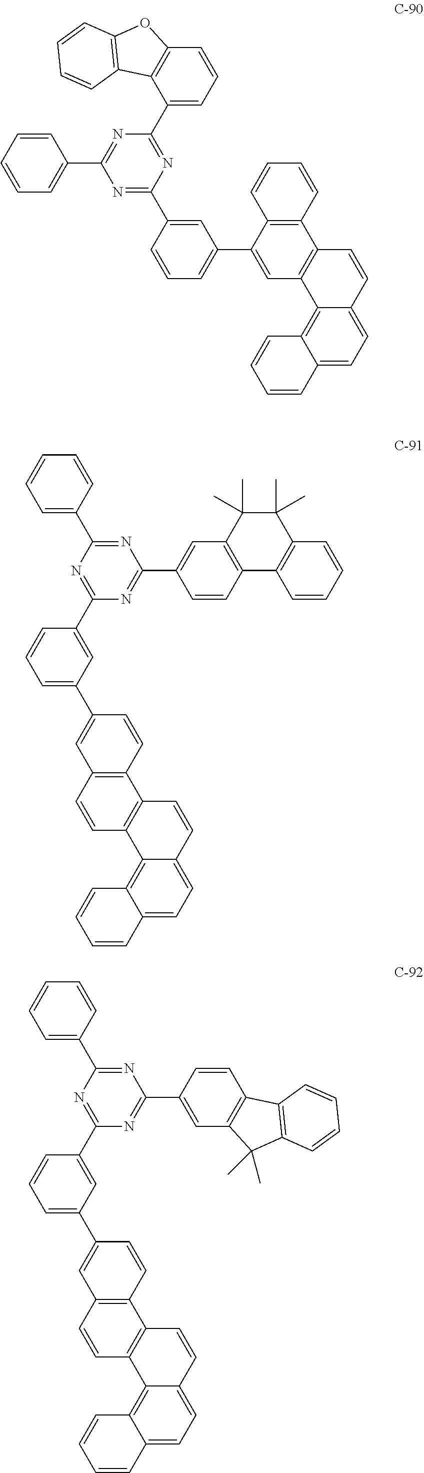

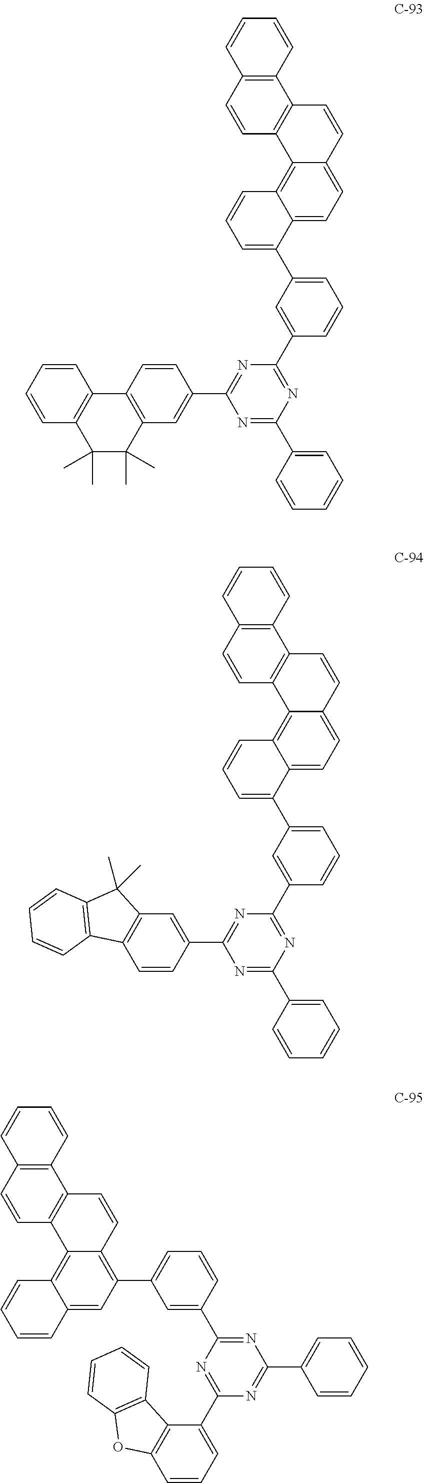 [SMILES: CC1(C)c2ccccc2-c2ccc(-c3nc(-c4ccccc4)nc(-c4cccc(-c5cccc6c5ccc5ccc7c8ccccc8ccc7c56)c4)n3)cc2C1(C)C ; CC1(C)c2ccccc2-c2ccc(-c3nc(-c4ccccc4)nc(-c4cccc(-c5cccc6c5ccc5ccc7c8ccccc8ccc7c56)c4)n3)cc21 ; c1ccc(-c2nc(-c3cccc(-c4cc5ccccc5c5c4ccc4c6ccccc6ccc45)c3)nc(-c3cccc4oc5ccccc5c34)n2)cc1]

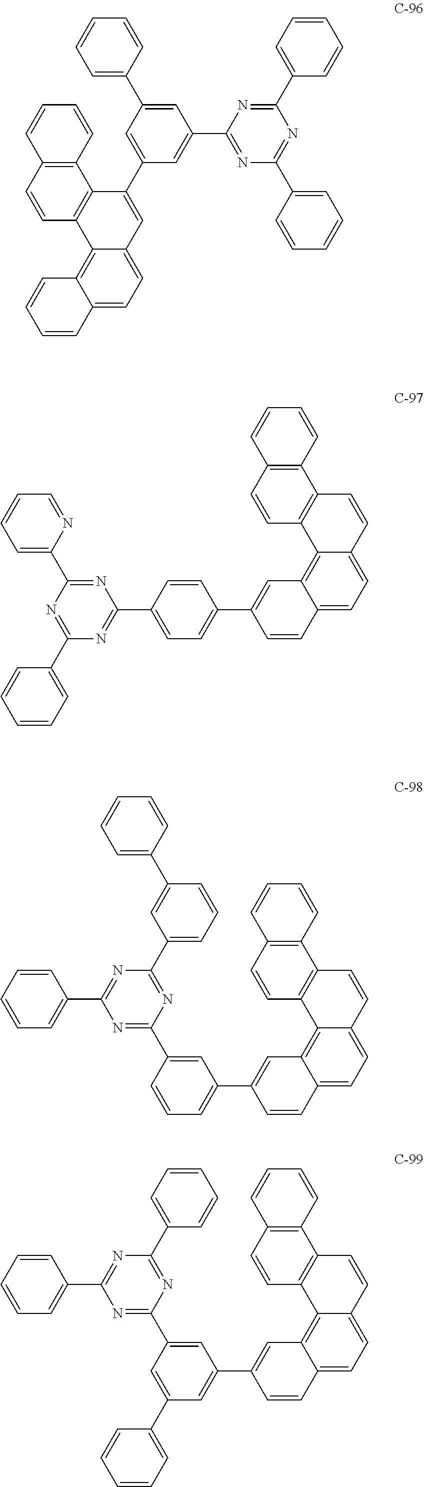 [SMILES: c1ccc(-c2cc(-c3nc(-c4ccccc4)nc(-c4ccccc4)n3)cc(-c3cc4ccc5ccccc5c4c4ccc5ccccc5c34)c2)cc1 ; c1ccc(-c2nc(-c3ccc(-c4ccc5ccc6ccc7c8ccccc8ccc7c6c5c4)cc3)nc(-c3ccccn3)n2)cc1 ; c1ccc(-c2cccc(-c3nc(-c4ccccc4)nc(-c4cccc(-c5ccc6ccc7ccc8c9ccccc9ccc8c7c6c5)c4)n3)c2)cc1 ; c1ccc(-c2cc(-c3ccc4ccc5ccc6c7ccccc7ccc6c5c4c3)cc(-c3nc(-c4ccccc4)nc(-c4ccccc4)n3)c2)cc1]

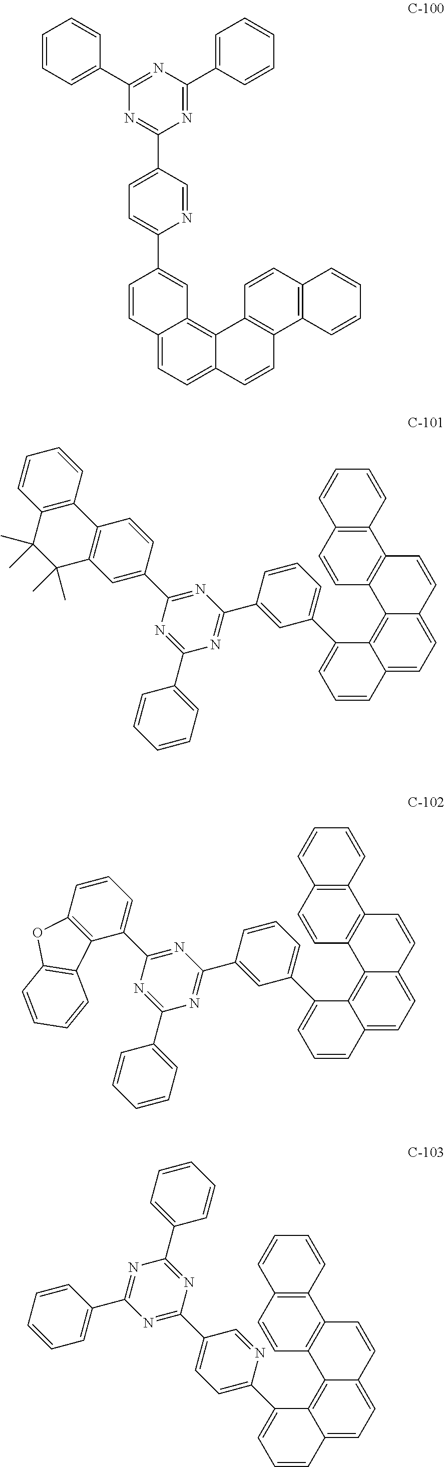 [SMILES: c1ccc(-c2nc(-c3ccccc3)nc(-c3ccc(-c4ccc5ccc6ccc7c8ccccc8ccc7c6c5c4)nc3)n2)cc1 ; CC1(C)c2ccccc2-c2ccc(-c3nc(-c4ccccc4)nc(-c4cccc(-c5cccc6ccc7ccc8c9ccccc9ccc8c7c56)c4)n3)cc2C1(C)C ; c1ccc(-c2nc(-c3cccc(-c4cccc5ccc6ccc7c8ccccc8ccc7c6c45)c3)nc(-c3cccc4oc5ccccc5c34)n2)cc1 ; c1ccc(-c2nc(-c3ccccc3)nc(-c3ccc(-c4cccc5ccc6ccc7c8ccccc8ccc7c6c45)nc3)n2)cc1]

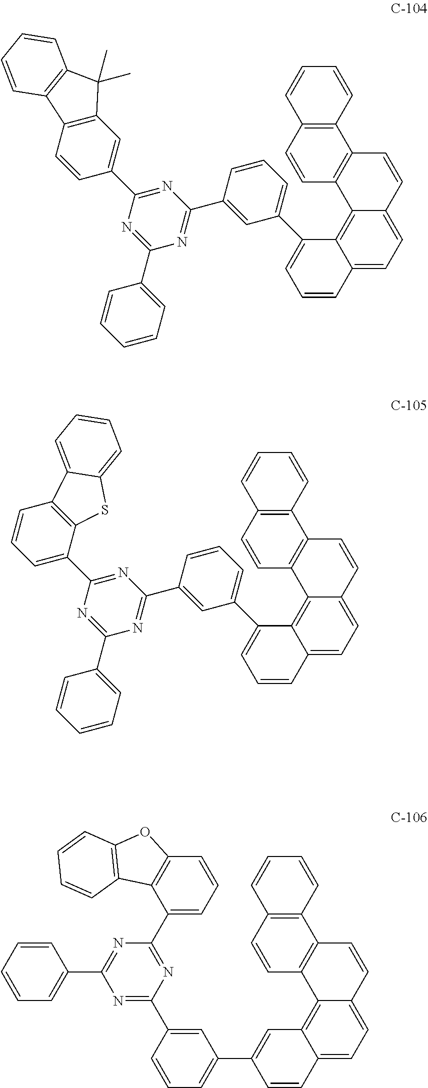

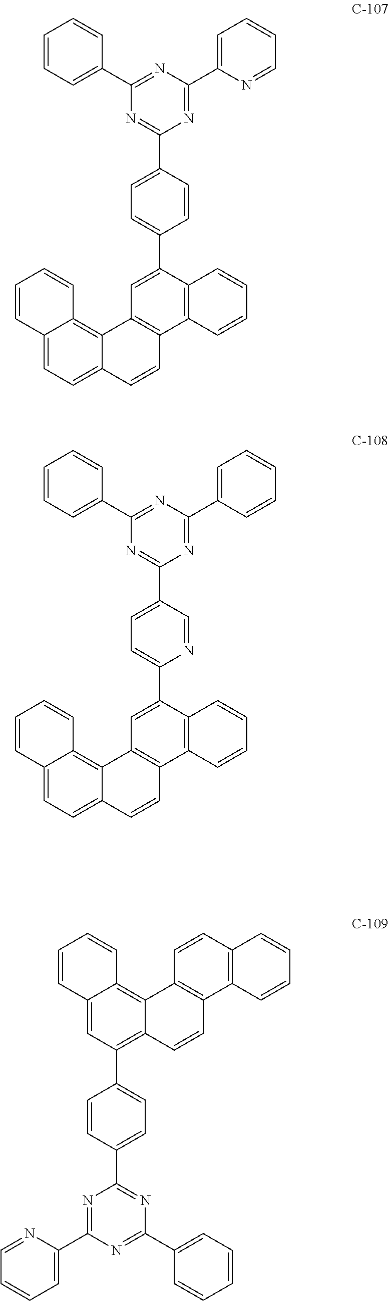 [SMILES: c1ccc(-c2nc(-c3ccc(-c4cc5c(ccc6ccc7ccccc7c65)c5ccccc45)cc3)nc(-c3ccccn3)n2)cc1 ; c1ccc(-c2nc(-c3ccccc3)nc(-c3ccc(-c4cc5c(ccc6ccc7ccccc7c65)c5ccccc45)nc3)n2)cc1 ; c1ccc(-c2nc(-c3ccc(-c4cc5ccccc5c5c4ccc4c6ccccc6ccc45)cc3)nc(-c3ccccn3)n2)cc1]

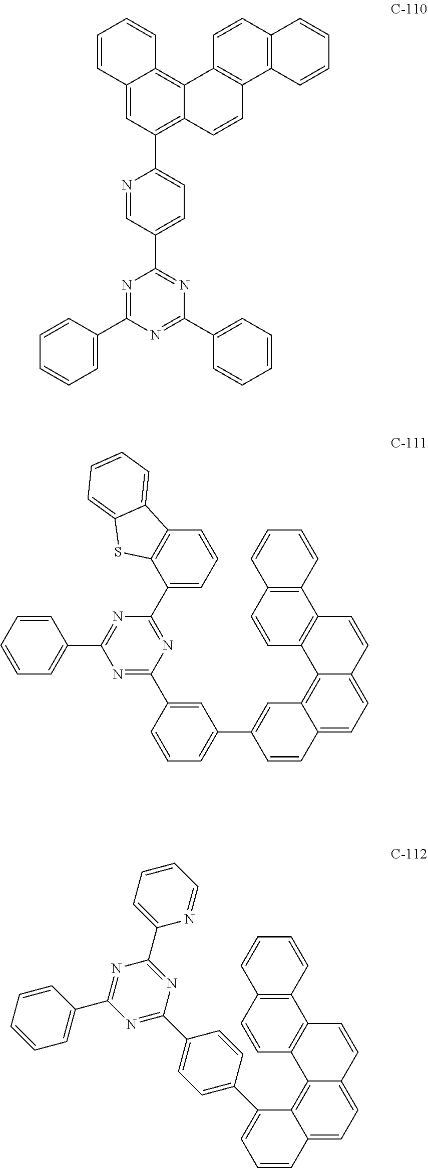 [SMILES: c1ccc(-c2nc(-c3ccccc3)nc(-c3ccc(-c4cc5ccccc5c5c4ccc4c6ccccc6ccc45)nc3)n2)cc1 ; c1ccc(-c2nc(-c3cccc(-c4ccc5ccc6ccc7c8ccccc8ccc7c6c5c4)c3)nc(-c3cccc4c3sc3ccccc34)n2)cc1 ; c1ccc(-c2nc(-c3ccc(-c4cccc5ccc6ccc7c8ccccc8ccc7c6c45)cc3)nc(-c3ccccn3)n2)cc1]

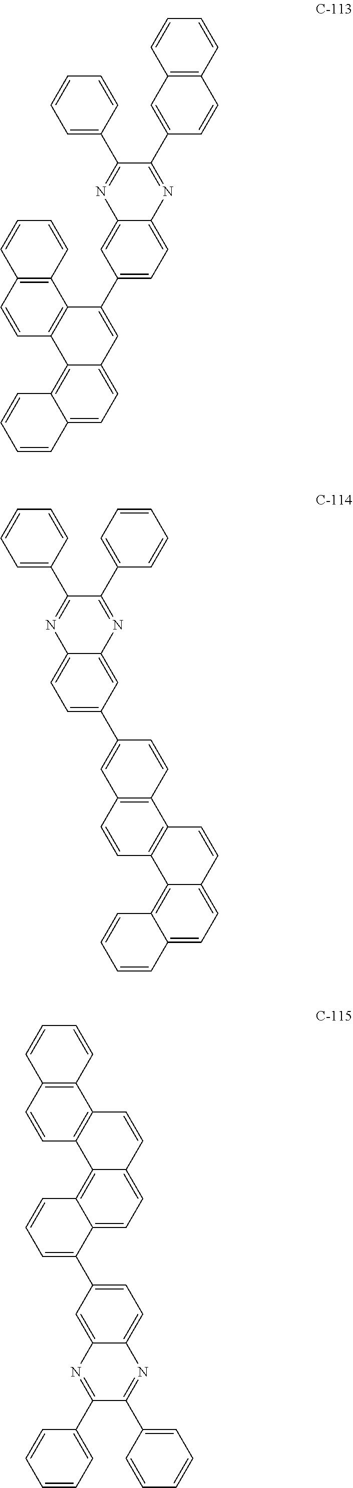 [SMILES: c1ccc(-c2nc3cc(-c4cc5ccc6ccccc6c5c5ccc6ccccc6c45)ccc3nc2-c2ccc3ccccc3c2)cc1 ; c1ccc(-c2nc3ccc(-c4ccc5c(ccc6c5ccc5ccc7ccccc7c56)c4)cc3nc2-c2ccccc2)cc1 ; c1ccc(-c2nc3ccc(-c4cccc5c4ccc4ccc6c7ccccc7ccc6c45)cc3nc2-c2ccccc2)cc1]

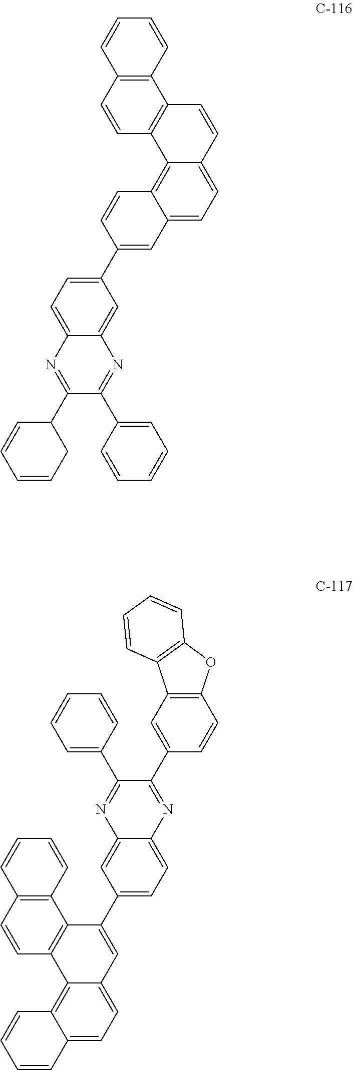 [SMILES: C1=CCC(c2nc3ccc(-c4ccc5c(ccc6ccc7c8ccccc8ccc7c65)c4)cc3nc2-c2ccccc2)C=C1 ; c1ccc(-c2nc3cc(-c4cc5ccc6ccccc6c5c5ccc6ccccc6c45)ccc3nc2-c2ccc3oc4ccccc4c3c2)cc1]

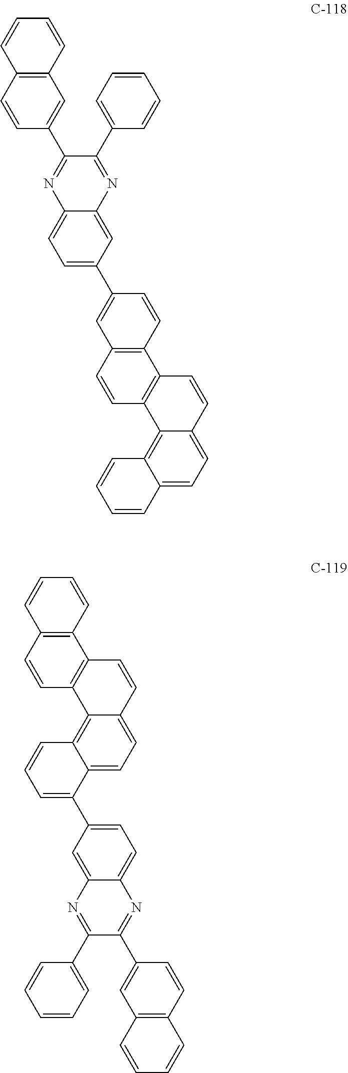 [SMILES: c1ccc(-c2nc3cc(-c4ccc5c(ccc6c5ccc5ccc7ccccc7c56)c4)ccc3nc2-c2ccc3ccccc3c2)cc1 ; c1ccc(-c2nc3cc(-c4cccc5c4ccc4ccc6c7ccccc7ccc6c45)ccc3nc2-c2ccc3ccccc3c2)cc1]

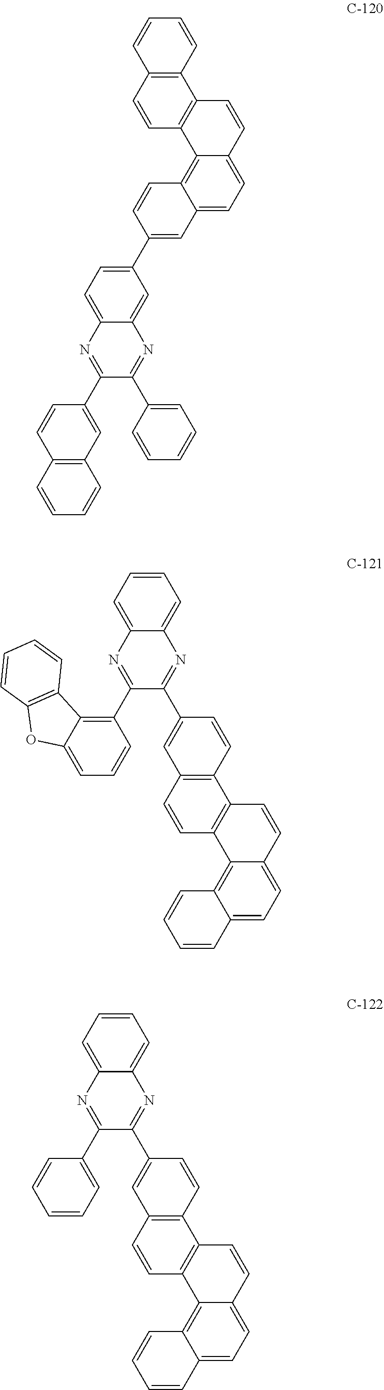 [SMILES: c1ccc(-c2nc3cc(-c4ccc5c(ccc6ccc7c8ccccc8ccc7c65)c4)ccc3nc2-c2ccc3ccccc3c2)cc1 ; c1ccc2c(c1)ccc1ccc3c4ccc(-c5nc6ccccc6nc5-c5cccc6oc7ccccc7c56)cc4ccc3c12 ; c1ccc(-c2nc3ccccc3nc2-c2ccc3c(ccc4c3ccc3ccc5ccccc5c34)c2)cc1]

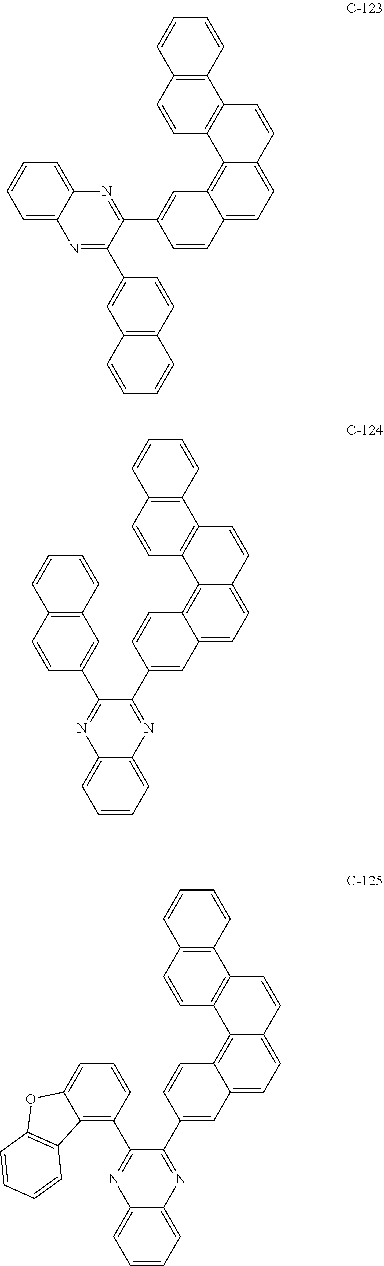 [SMILES: c1ccc2cc(-c3nc4ccccc4nc3-c3ccc4ccc5ccc6c7ccccc7ccc6c5c4c3)ccc2c1 ; c1ccc2cc(-c3nc4ccccc4nc3-c3ccc4c(ccc5ccc6c7ccccc7ccc6c54)c3)ccc2c1 ; c1ccc2c(c1)ccc1c2ccc2ccc3cc(-c4nc5ccccc5nc4-c4cccc5oc6ccccc6c45)ccc3c21]

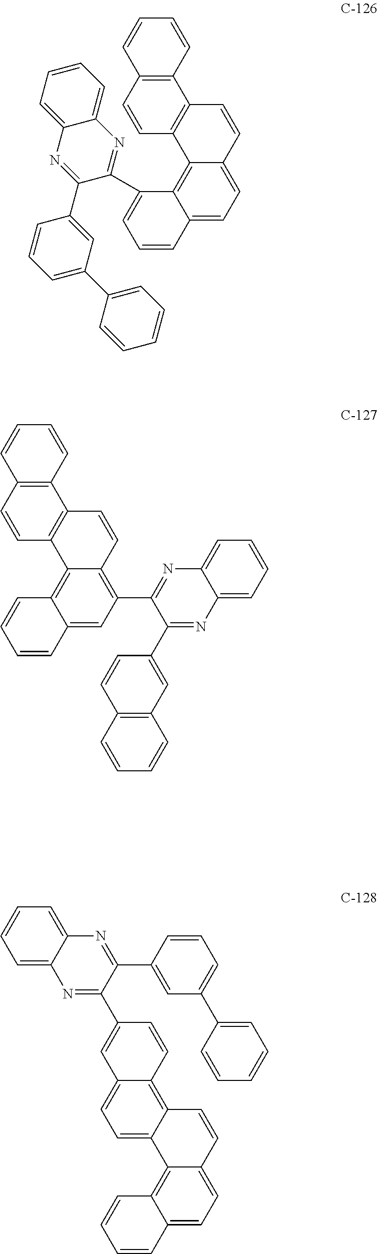 [SMILES: c1ccc(-c2cccc(-c3nc4ccccc4nc3-c3cccc4ccc5ccc6c7ccccc7ccc6c5c34)c2)cc1 ; c1ccc2cc(-c3nc4ccccc4nc3-c3cc4ccccc4c4c3ccc3c5ccccc5ccc34)ccc2c1 ; c1ccc(-c2cccc(-c3nc4ccccc4nc3-c3ccc4c(ccc5c4ccc4ccc6ccccc6c45)c3)c2)cc1]

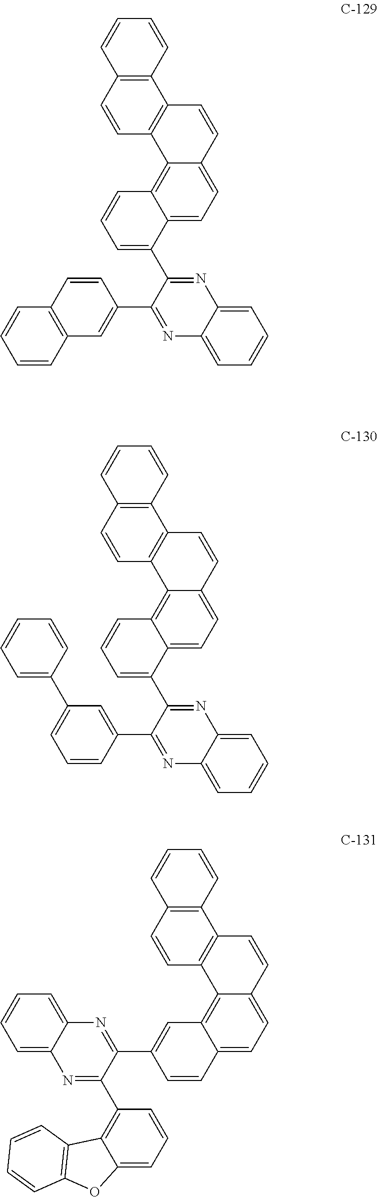 [SMILES: c1ccc2cc(-c3nc4ccccc4nc3-c3cccc4c3ccc3ccc5c6ccccc6ccc5c34)ccc2c1 ; c1ccc(-c2cccc(-c3nc4ccccc4nc3-c3cccc4c3ccc3ccc5c6ccccc6ccc5c34)c2)cc1 ; c1ccc2c(c1)ccc1c2ccc2ccc3ccc(-c4nc5ccccc5nc4-c4cccc5oc6ccccc6c45)cc3c21]

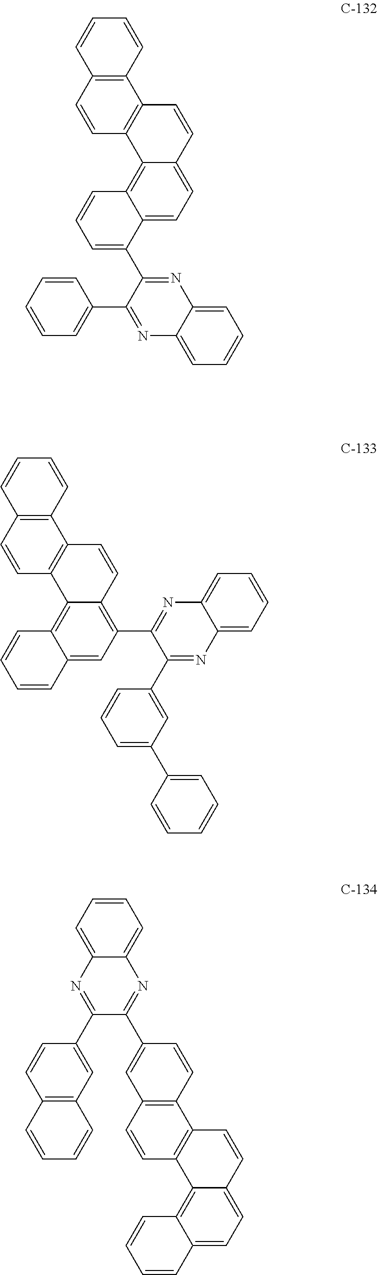 [SMILES: c1ccc(-c2nc3ccccc3nc2-c2cccc3c2ccc2ccc4c5ccccc5ccc4c23)cc1 ; c1ccc(-c2cccc(-c3nc4ccccc4nc3-c3cc4ccccc4c4c3ccc3c5ccccc5ccc34)c2)cc1 ; c1ccc2cc(-c3nc4ccccc4nc3-c3ccc4c(ccc5c4ccc4ccc6ccccc6c45)c3)ccc2c1]

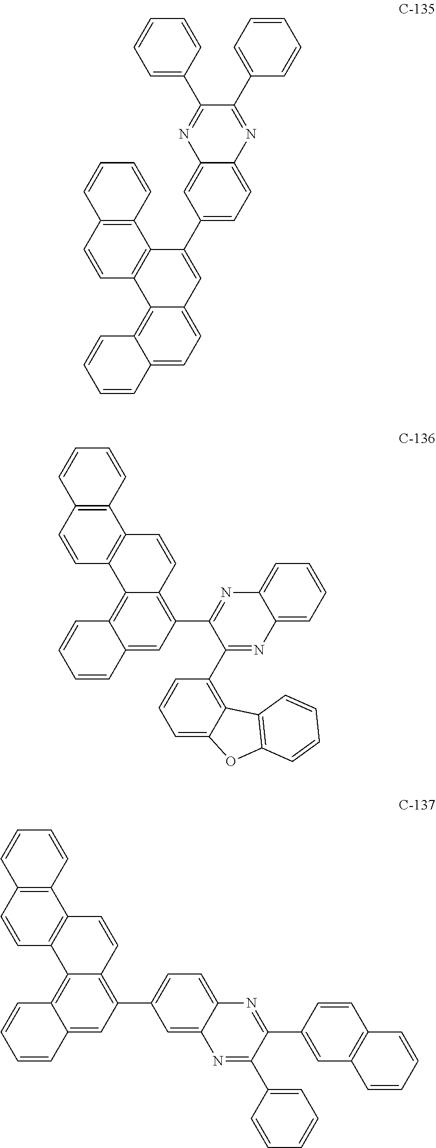

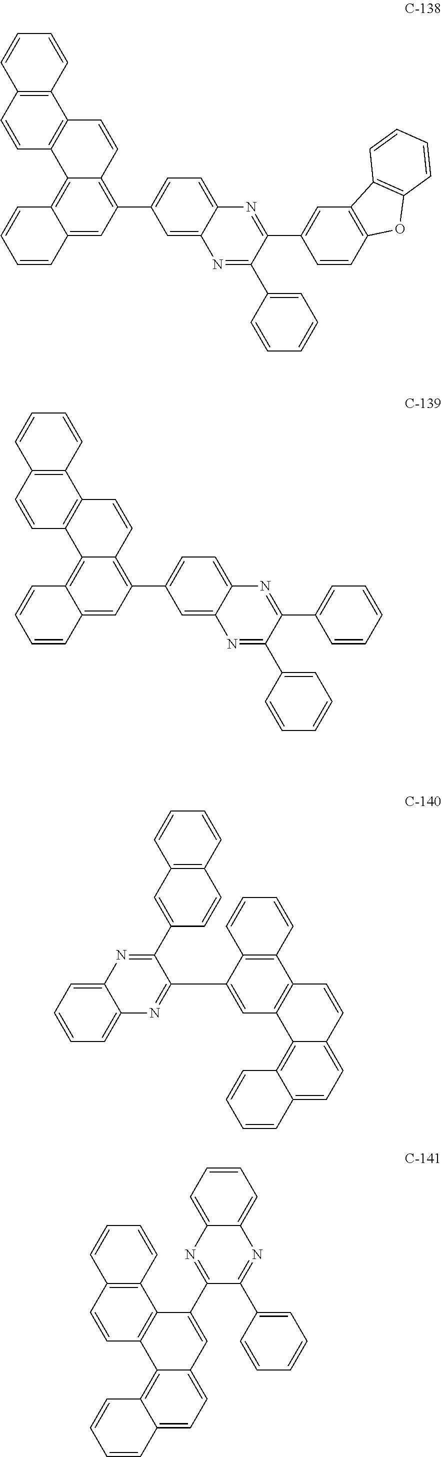

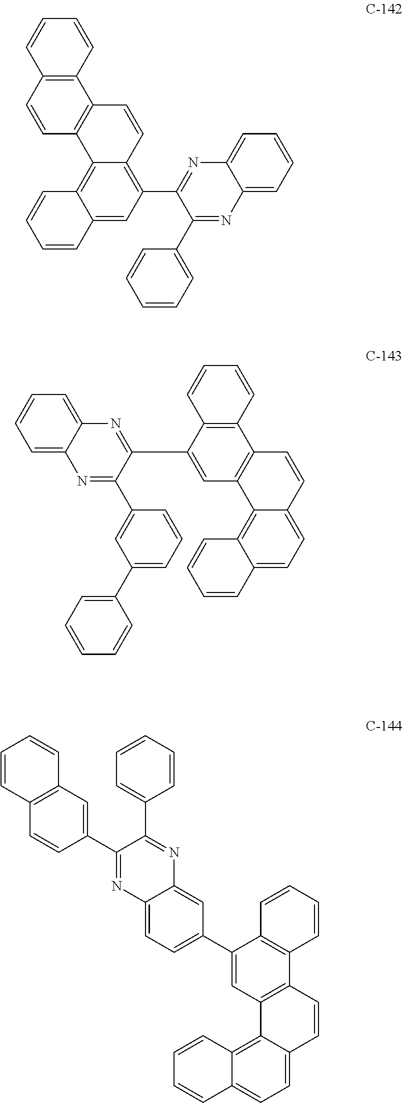

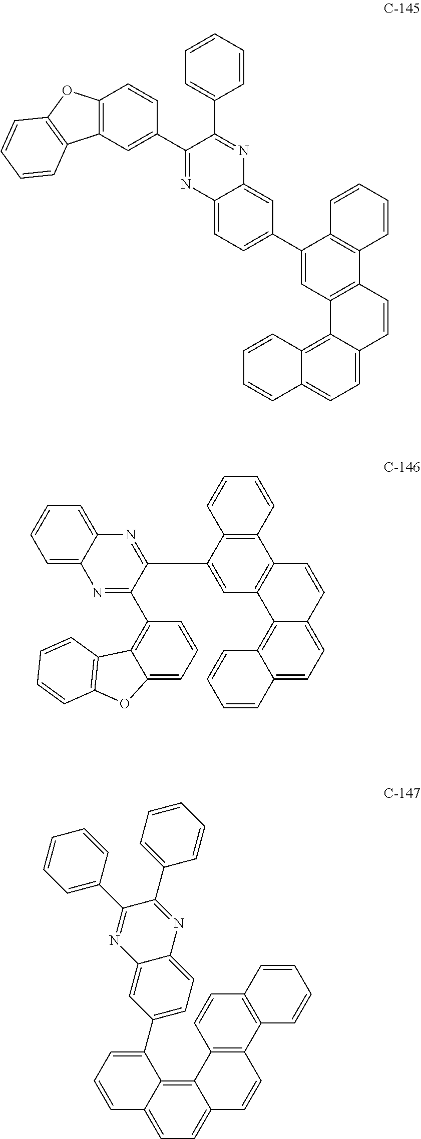 [SMILES: c1ccc(-c2nc3cc(-c4cc5c(ccc6ccc7ccccc7c65)c5ccccc45)ccc3nc2-c2ccc3oc4ccccc4c3c2)cc1 ; c1ccc2c(c1)ccc1ccc3c4ccccc4c(-c4nc5ccccc5nc4-c4cccc5oc6ccccc6c45)cc3c12 ; c1ccc(-c2nc3ccc(-c4cccc5ccc6ccc7c8ccccc8ccc7c6c45)cc3nc2-c2ccccc2)cc1]

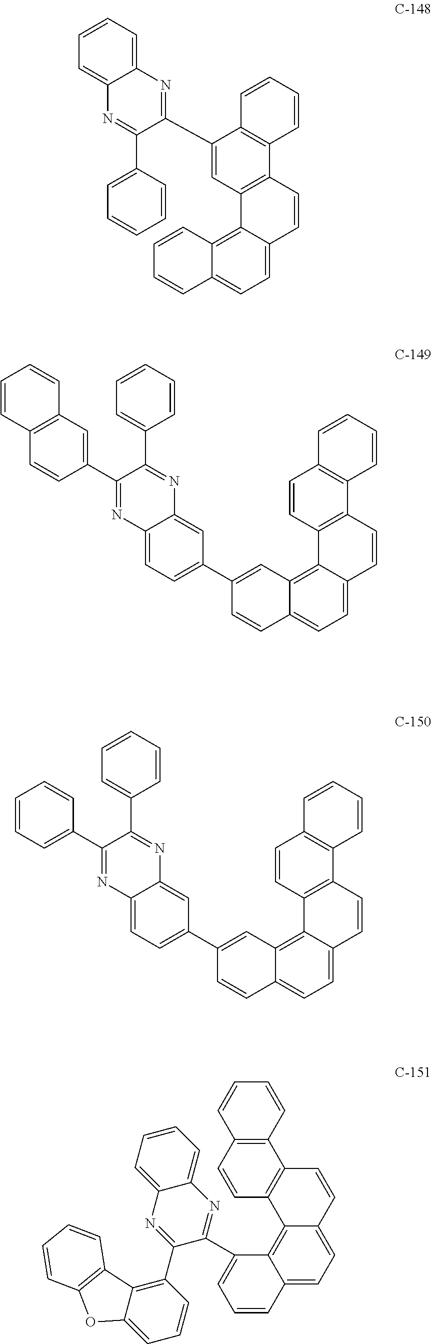 [SMILES: c1ccc(-c2nc3ccccc3nc2-c2cc3c(ccc4ccc5ccccc5c43)c3ccccc23)cc1 ; c1ccc(-c2nc3cc(-c4ccc5ccc6ccc7c8ccccc8ccc7c6c5c4)ccc3nc2-c2ccc3ccccc3c2)cc1 ; c1ccc(-c2nc3ccc(-c4ccc5ccc6ccc7c8ccccc8ccc7c6c5c4)cc3nc2-c2ccccc2)cc1 ; c1ccc2c(c1)ccc1c2ccc2ccc3cccc(-c4nc5ccccc5nc4-c4cccc5oc6ccccc6c45)c3c21]

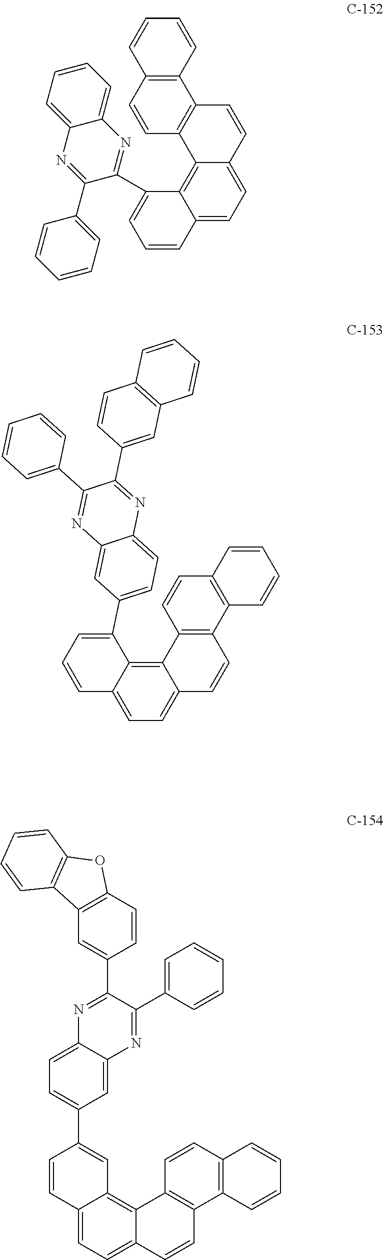 [SMILES: c1ccc(-c2nc3ccccc3nc2-c2cccc3ccc4ccc5c6ccccc6ccc5c4c23)cc1 ; c1ccc(-c2nc3cc(-c4cccc5ccc6ccc7c8ccccc8ccc7c6c45)ccc3nc2-c2ccc3ccccc3c2)cc1 ; c1ccc(-c2nc3cc(-c4ccc5ccc6ccc7c8ccccc8ccc7c6c5c4)ccc3nc2-c2ccc3oc4ccccc4c3c2)cc1]

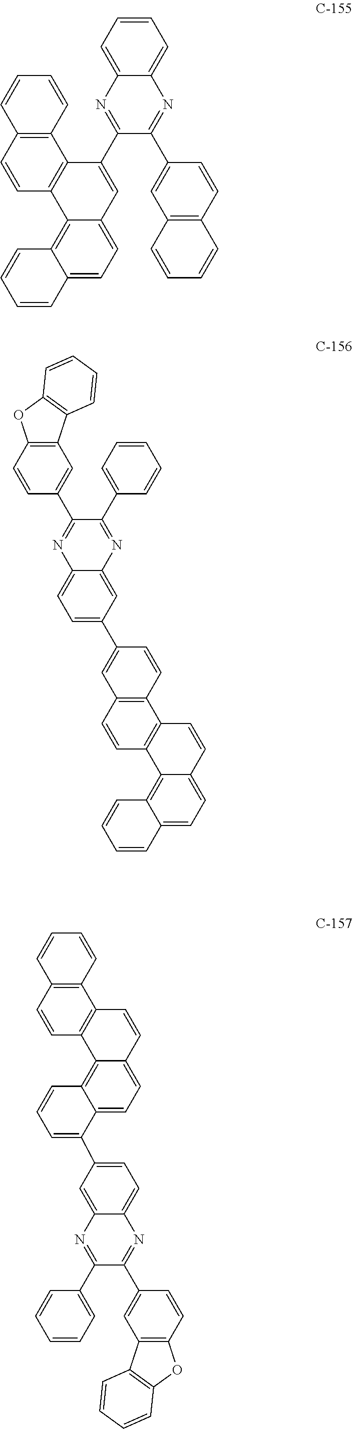 [SMILES: c1ccc2cc(-c3nc4ccccc4nc3-c3cc4ccc5ccccc5c4c4ccc5ccccc5c34)ccc2c1 ; c1ccc(-c2nc3cc(-c4ccc5c(ccc6c5ccc5ccc7ccccc7c56)c4)ccc3nc2-c2ccc3oc4ccccc4c3c2)cc1 ; c1ccc(-c2nc3cc(-c4cccc5c4ccc4ccc6c7ccccc7ccc6c45)ccc3nc2-c2ccc3oc4ccccc4c3c2)cc1]

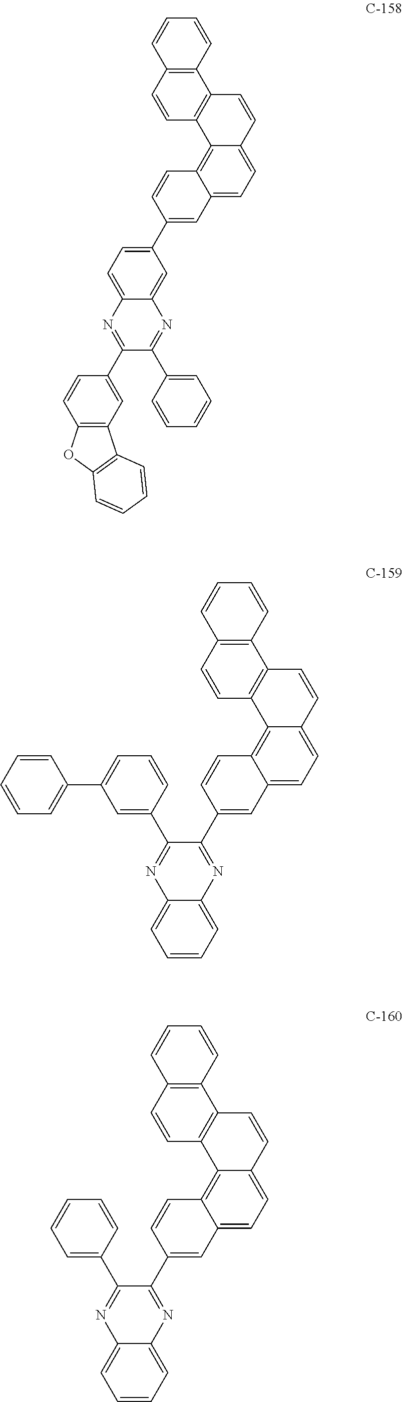 [SMILES: c1ccc(-c2nc3cc(-c4ccc5c(ccc6ccc7c8ccccc8ccc7c65)c4)ccc3nc2-c2ccc3oc4ccccc4c3c2)cc1 ; c1ccc(-c2cccc(-c3nc4ccccc4nc3-c3ccc4c(ccc5ccc6c7ccccc7ccc6c54)c3)c2)cc1 ; c1ccc(-c2nc3ccccc3nc2-c2ccc3c(ccc4ccc5c6ccccc6ccc5c43)c2)cc1]

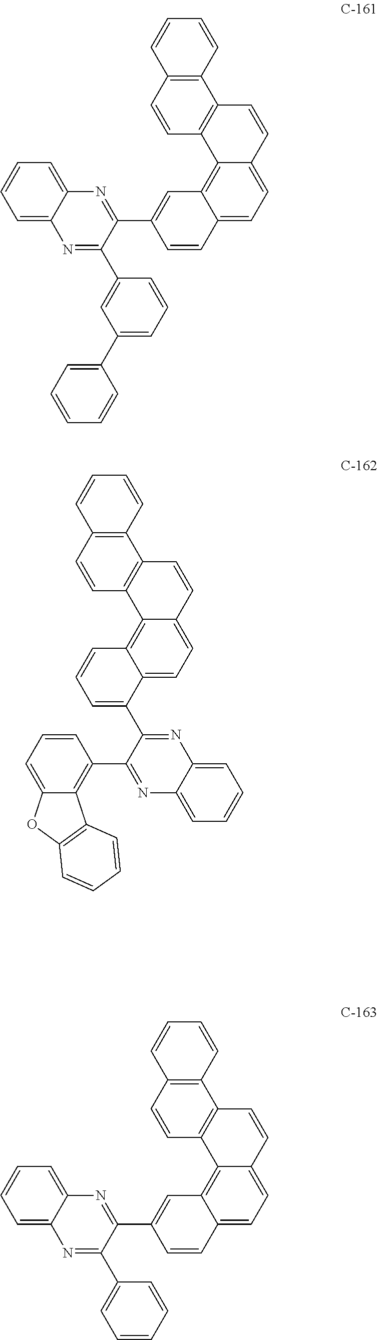 [SMILES: c1ccc(-c2cccc(-c3nc4ccccc4nc3-c3ccc4ccc5ccc6c7ccccc7ccc6c5c4c3)c2)cc1 ; c1ccc2c(c1)ccc1c2ccc2ccc3c(-c4nc5ccccc5nc4-c4cccc5oc6ccccc6c45)cccc3c21 ; c1ccc(-c2nc3ccccc3nc2-c2ccc3ccc4ccc5c6ccccc6ccc5c4c3c2)cc1]

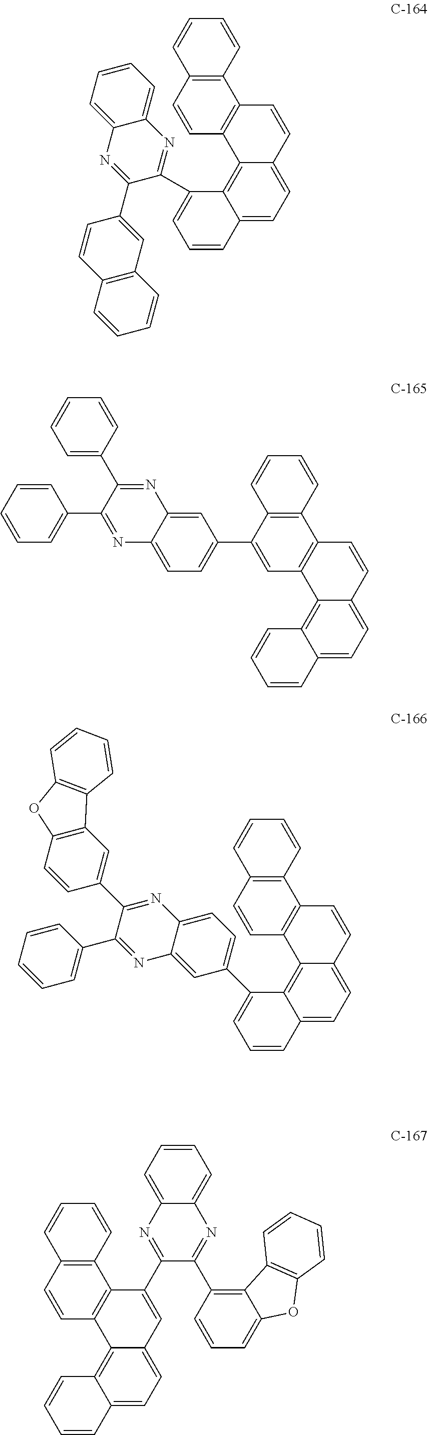 [SMILES: c1ccc2cc(-c3nc4ccccc4nc3-c3cccc4ccc5ccc6c7ccccc7ccc6c5c34)ccc2c1 ; c1ccc(-c2nc3ccc(-c4cc5c(ccc6ccc7ccccc7c65)c5ccccc45)cc3nc2-c2ccccc2)cc1 ; c1ccc(-c2nc3cc(-c4cccc5ccc6ccc7c8ccccc8ccc7c6c45)ccc3nc2-c2ccc3oc4ccccc4c3c2)cc1 ; c1ccc2c(c1)ccc1c2c(-c2nc3ccccc3nc2-c2cccc3oc4ccccc4c23)cc2ccc3ccccc3c21]

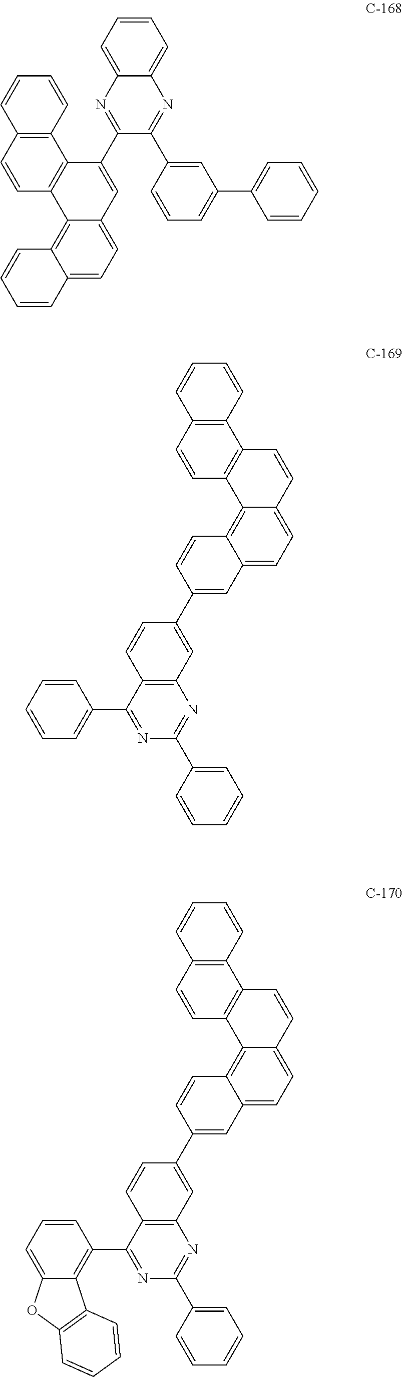 [SMILES: c1ccc(-c2cccc(-c3nc4ccccc4nc3-c3cc4ccc5ccccc5c4c4ccc5ccccc5c34)c2)cc1 ; c1ccc(-c2nc(-c3ccccc3)c3ccc(-c4ccc5c(ccc6ccc7c8ccccc8ccc7c65)c4)cc3n2)cc1 ; c1ccc(-c2nc(-c3cccc4oc5ccccc5c34)c3ccc(-c4ccc5c(ccc6ccc7c8ccccc8ccc7c65)c4)cc3n2)cc1]

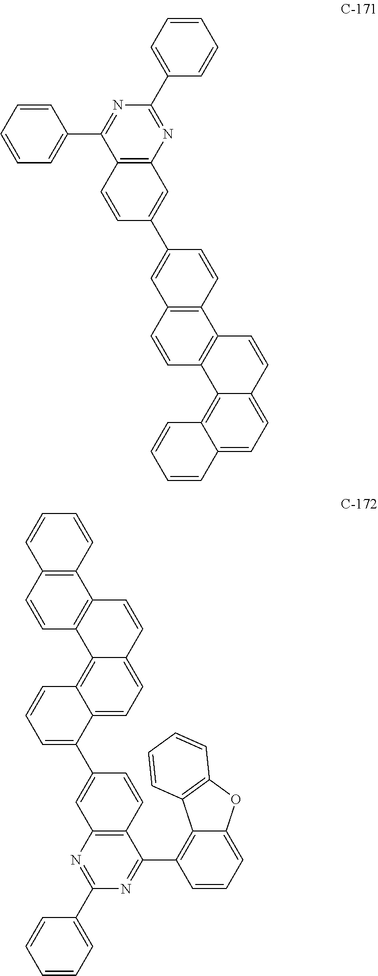 [SMILES: c1ccc(-c2nc(-c3ccccc3)c3ccc(-c4ccc5c(ccc6c5ccc5ccc7ccccc7c56)c4)cc3n2)cc1 ; c1ccc(-c2nc(-c3cccc4oc5ccccc5c34)c3ccc(-c4cccc5c4ccc4ccc6c7ccccc7ccc6c45)cc3n2)cc1]

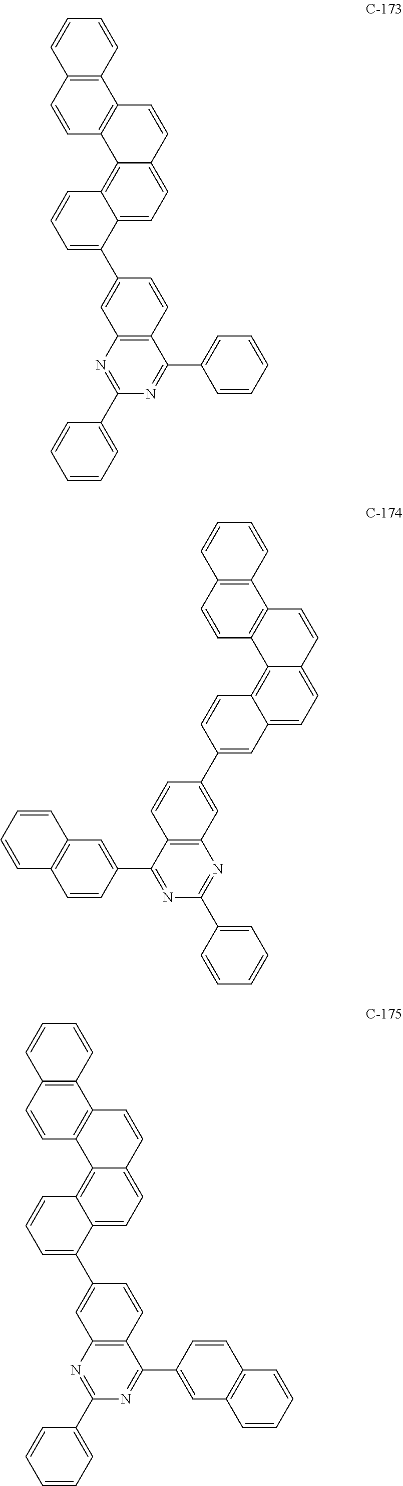 [SMILES: c1ccc(-c2nc(-c3ccccc3)c3ccc(-c4cccc5c4ccc4ccc6c7ccccc7ccc6c45)cc3n2)cc1 ; c1ccc(-c2nc(-c3ccc4ccccc4c3)c3ccc(-c4ccc5c(ccc6ccc7c8ccccc8ccc7c65)c4)cc3n2)cc1 ; c1ccc(-c2nc(-c3ccc4ccccc4c3)c3ccc(-c4cccc5c4ccc4ccc6c7ccccc7ccc6c45)cc3n2)cc1]

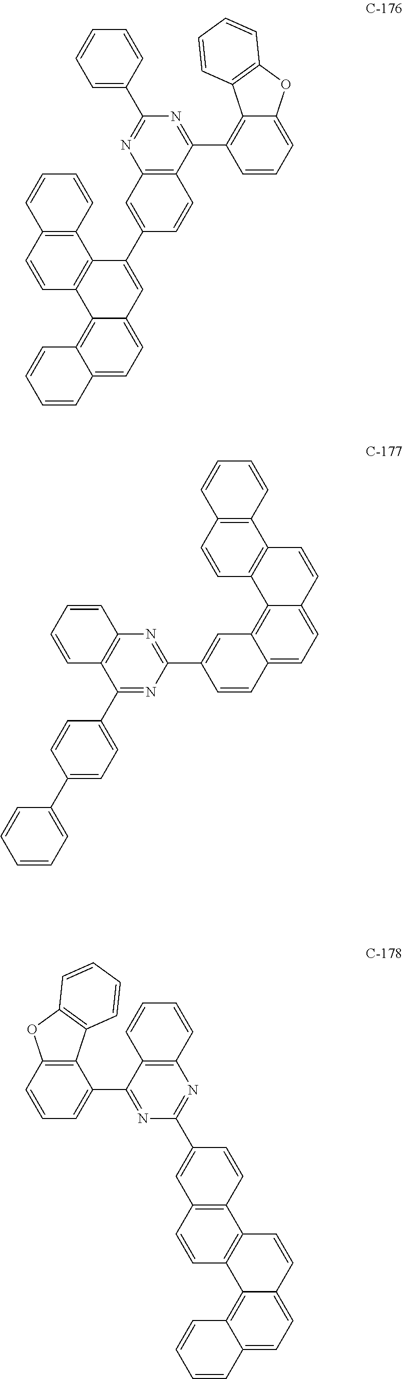 [SMILES: c1ccc(-c2nc(-c3cccc4oc5ccccc5c34)c3ccc(-c4cc5ccc6ccccc6c5c5ccc6ccccc6c45)cc3n2)cc1 ; c1ccc(-c2ccc(-c3nc(-c4ccc5ccc6ccc7c8ccccc8ccc7c6c5c4)nc4ccccc34)cc2)cc1 ; c1ccc2c(c1)ccc1ccc3c4ccc(-c5nc(-c6cccc7oc8ccccc8c67)c6ccccc6n5)cc4ccc3c12]

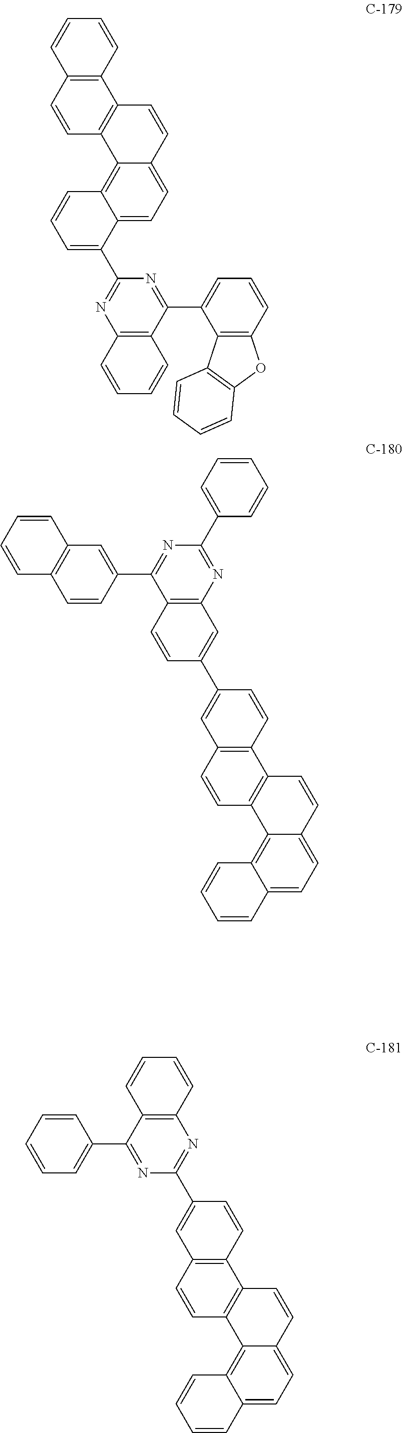

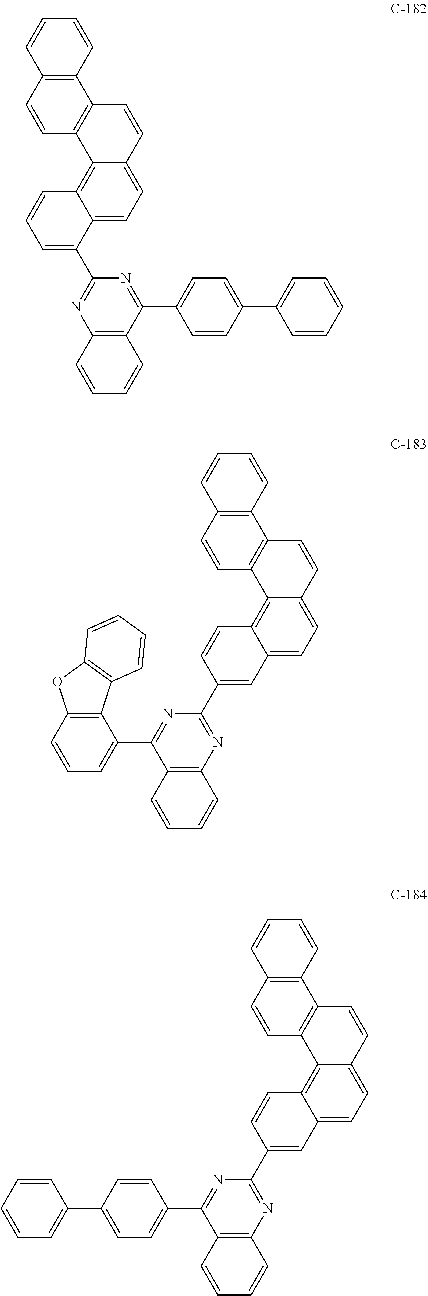 [SMILES: c1ccc(-c2ccc(-c3nc(-c4cccc5c4ccc4ccc6c7ccccc7ccc6c45)nc4ccccc34)cc2)cc1 ; c1ccc2c(c1)ccc1c2ccc2ccc3cc(-c4nc(-c5cccc6oc7ccccc7c56)c5ccccc5n4)ccc3c21 ; c1ccc(-c2ccc(-c3nc(-c4ccc5c(ccc6ccc7c8ccccc8ccc7c65)c4)nc4ccccc34)cc2)cc1]

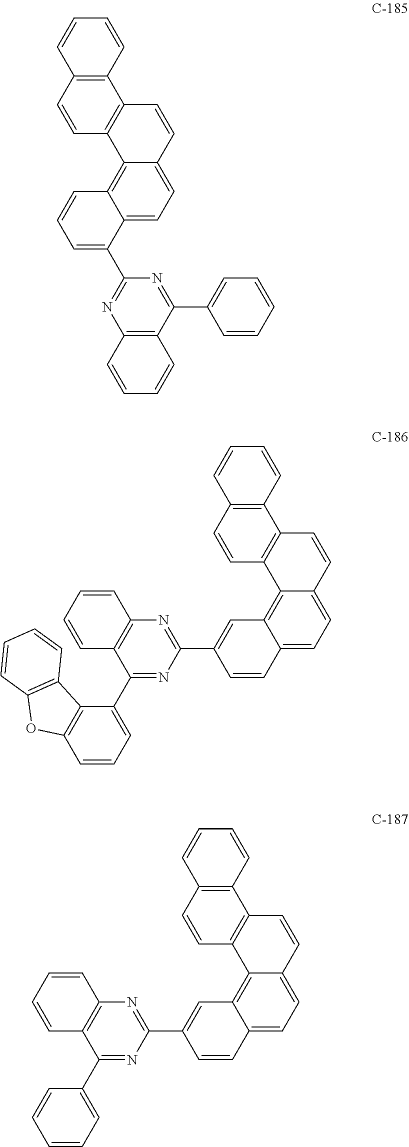 [SMILES: c1ccc(-c2nc(-c3cccc4c3ccc3ccc5c6ccccc6ccc5c34)nc3ccccc23)cc1 ; c1ccc2c(c1)ccc1c2ccc2ccc3ccc(-c4nc(-c5cccc6oc7ccccc7c56)c5ccccc5n4)cc3c21 ; c1ccc(-c2nc(-c3ccc4ccc5ccc6c7ccccc7ccc6c5c4c3)nc3ccccc23)cc1]

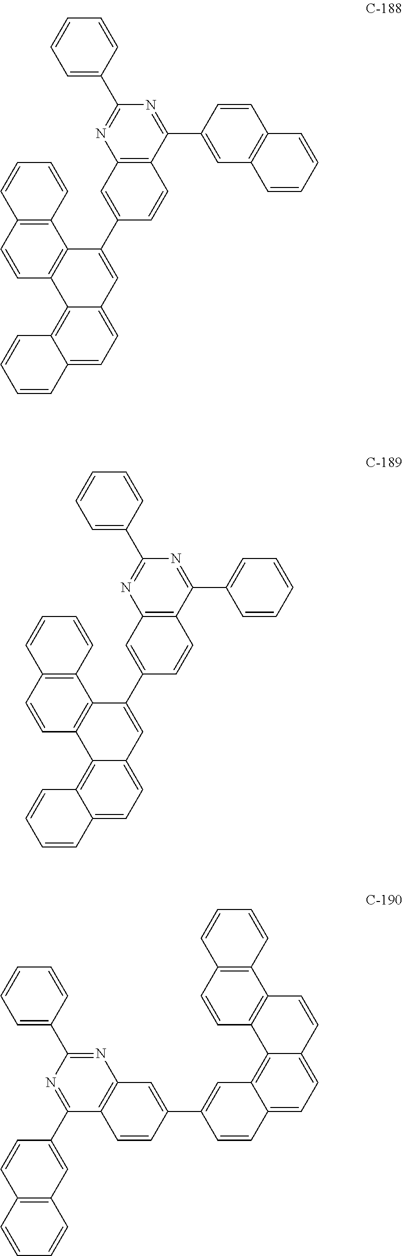 [SMILES: c1ccc(-c2nc(-c3ccc4ccccc4c3)c3ccc(-c4cc5ccc6ccccc6c5c5ccc6ccccc6c45)cc3n2)cc1 ; c1ccc(-c2nc(-c3ccccc3)c3ccc(-c4cc5ccc6ccccc6c5c5ccc6ccccc6c45)cc3n2)cc1 ; c1ccc(-c2nc(-c3ccc4ccccc4c3)c3ccc(-c4ccc5ccc6ccc7c8ccccc8ccc7c6c5c4)cc3n2)cc1]

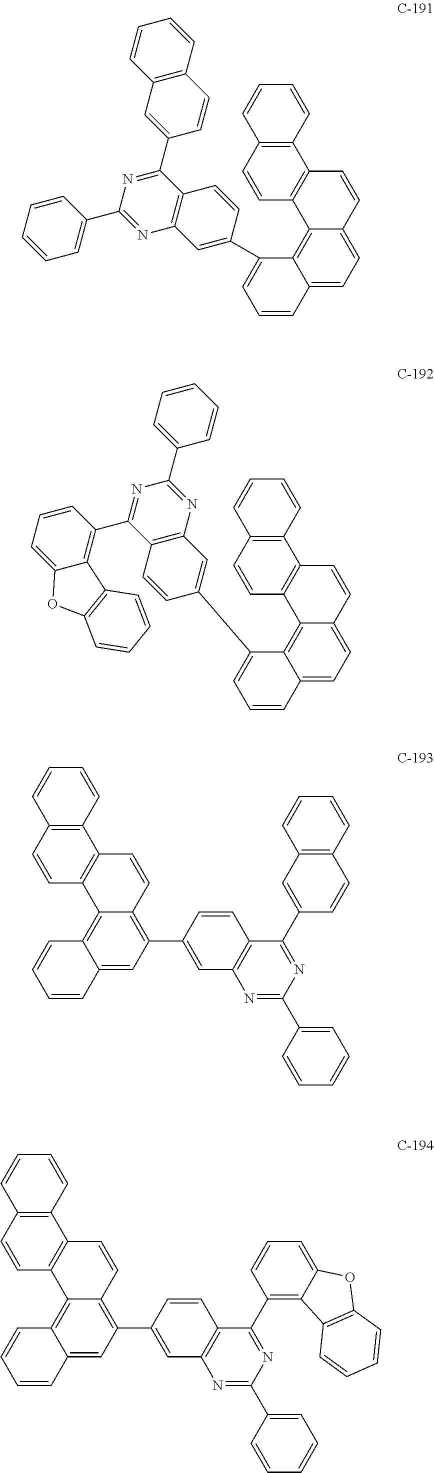 [SMILES: c1ccc(-c2nc(-c3ccc4ccccc4c3)c3ccc(-c4cccc5ccc6ccc7c8ccccc8ccc7c6c45)cc3n2)cc1 ; c1ccc(-c2nc(-c3cccc4oc5ccccc5c34)c3ccc(-c4cccc5ccc6ccc7c8ccccc8ccc7c6c45)cc3n2)cc1 ; c1ccc(-c2nc(-c3ccc4ccccc4c3)c3ccc(-c4cc5ccccc5c5c4ccc4c6ccccc6ccc45)cc3n2)cc1 ; c1ccc(-c2nc(-c3cccc4oc5ccccc5c34)c3ccc(-c4cc5ccccc5c5c4ccc4c6ccccc6ccc45)cc3n2)cc1]

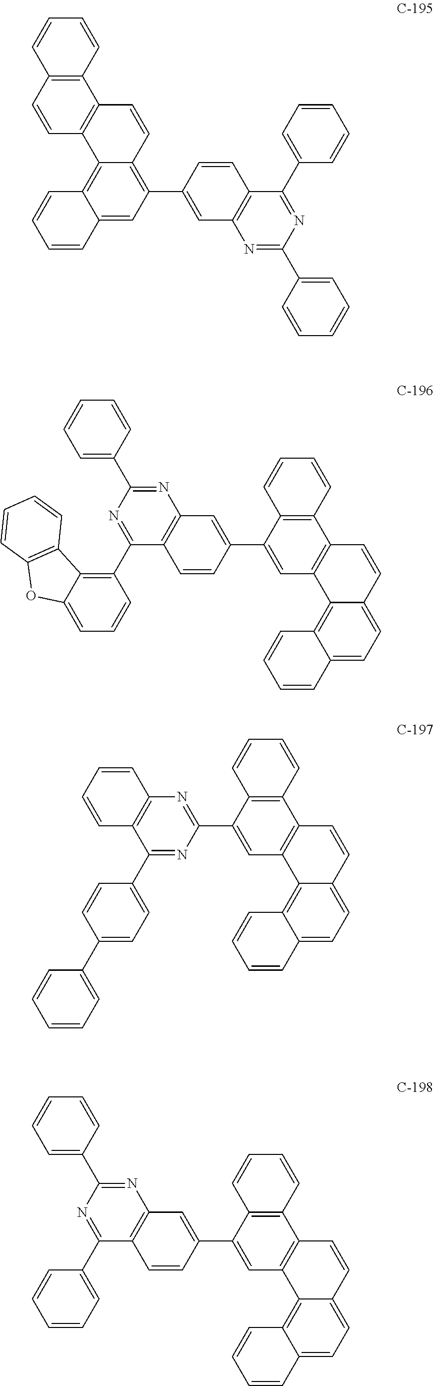 [SMILES: c1ccc(-c2nc(-c3ccccc3)c3ccc(-c4cc5ccccc5c5c4ccc4c6ccccc6ccc45)cc3n2)cc1 ; c1ccc(-c2nc(-c3cccc4oc5ccccc5c34)c3ccc(-c4cc5c(ccc6ccc7ccccc7c65)c5ccccc45)cc3n2)cc1 ; c1ccc(-c2ccc(-c3nc(-c4cc5c(ccc6ccc7ccccc7c65)c5ccccc45)nc4ccccc34)cc2)cc1 ; c1ccc(-c2nc(-c3ccccc3)c3ccc(-c4cc5c(ccc6ccc7ccccc7c65)c5ccccc45)cc3n2)cc1]

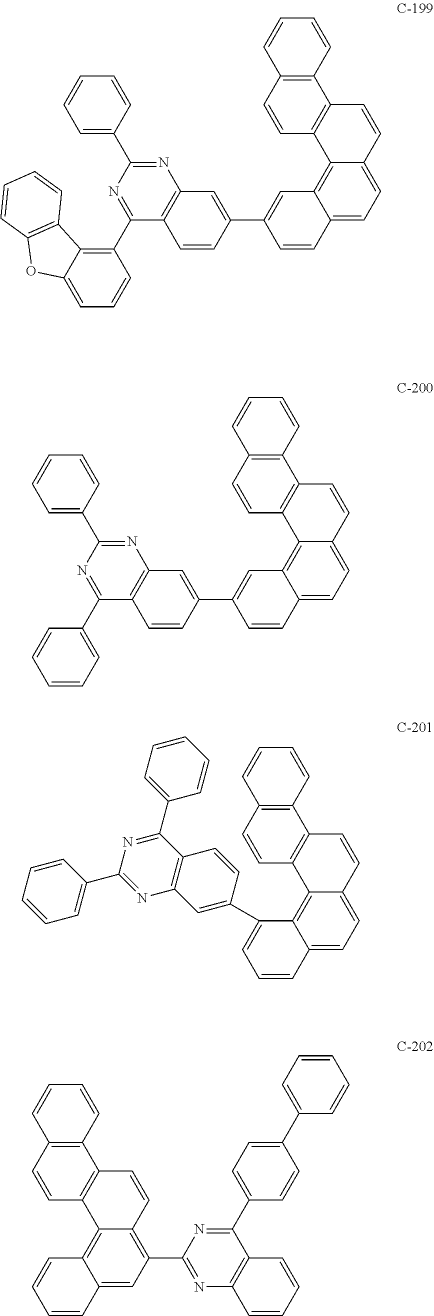 [SMILES: c1ccc(-c2nc(-c3cccc4oc5ccccc5c34)c3ccc(-c4ccc5ccc6ccc7c8ccccc8ccc7c6c5c4)cc3n2)cc1 ; c1ccc(-c2nc(-c3ccccc3)c3ccc(-c4ccc5ccc6ccc7c8ccccc8ccc7c6c5c4)cc3n2)cc1 ; c1ccc(-c2nc(-c3ccccc3)c3ccc(-c4cccc5ccc6ccc7c8ccccc8ccc7c6c45)cc3n2)cc1 ; c1ccc(-c2ccc(-c3nc(-c4cc5ccccc5c5c4ccc4c6ccccc6ccc45)nc4ccccc34)cc2)cc1]

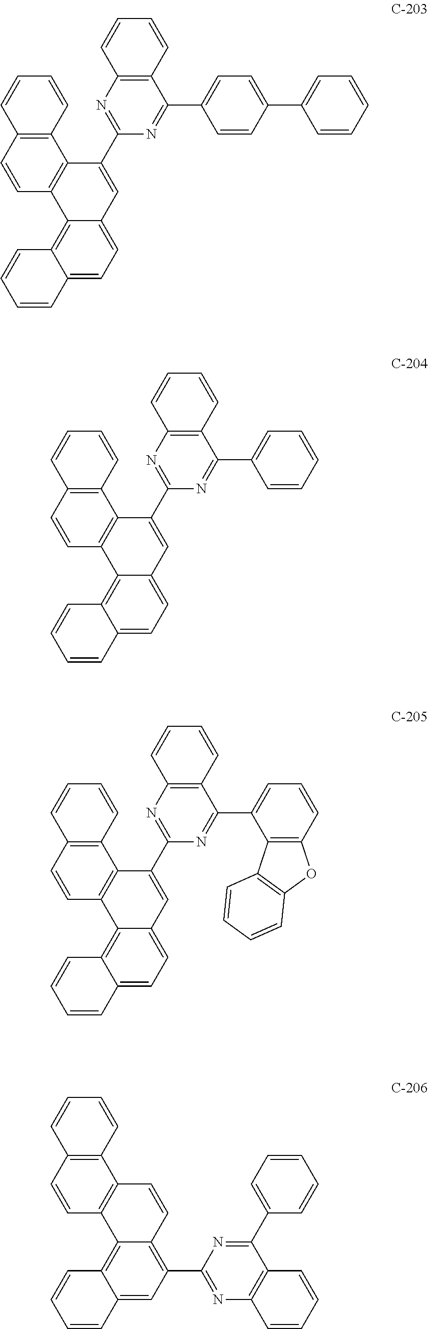 [SMILES: c1ccc(-c2ccc(-c3nc(-c4cc5ccc6ccccc6c5c5ccc6ccccc6c45)nc4ccccc34)cc2)cc1 ; c1ccc(-c2nc(-c3cc4ccc5ccccc5c4c4ccc5ccccc5c34)nc3ccccc23)cc1 ; c1ccc2c(c1)ccc1c2c(-c2nc(-c3cccc4oc5ccccc5c34)c3ccccc3n2)cc2ccc3ccccc3c21 ; c1ccc(-c2nc(-c3cc4ccccc4c4c3ccc3c5ccccc5ccc34)nc3ccccc23)cc1]

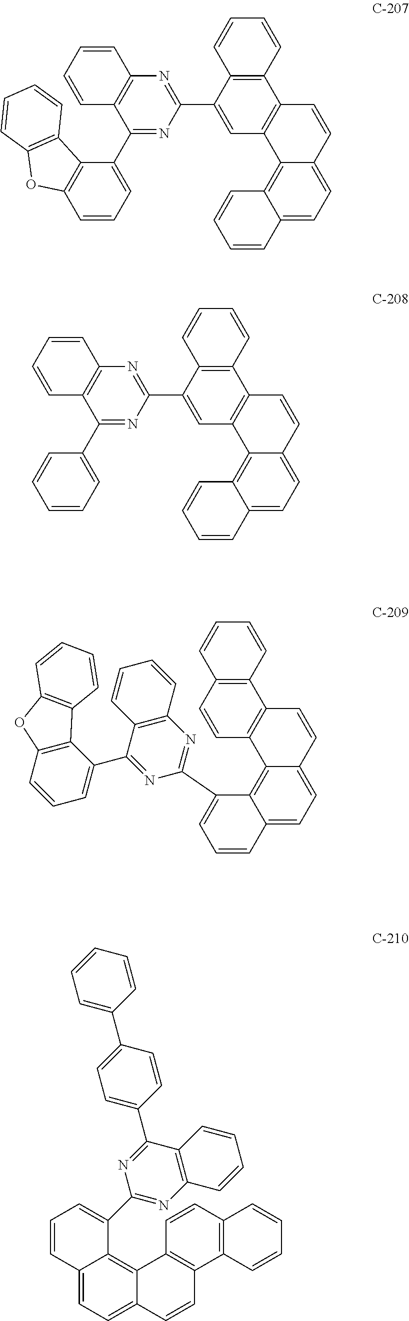 [SMILES: c1ccc2c(c1)ccc1ccc3c4ccccc4c(-c4nc(-c5cccc6oc7ccccc7c56)c5ccccc5n4)cc3c12 ; c1ccc(-c2nc(-c3cc4c(ccc5ccc6ccccc6c54)c4ccccc34)nc3ccccc23)cc1 ; c1ccc2c(c1)ccc1c2ccc2ccc3cccc(-c4nc(-c5cccc6oc7ccccc7c56)c5ccccc5n4)c3c21 ; c1ccc(-c2ccc(-c3nc(-c4cccc5ccc6ccc7c8ccccc8ccc7c6c45)nc4ccccc34)cc2)cc1]

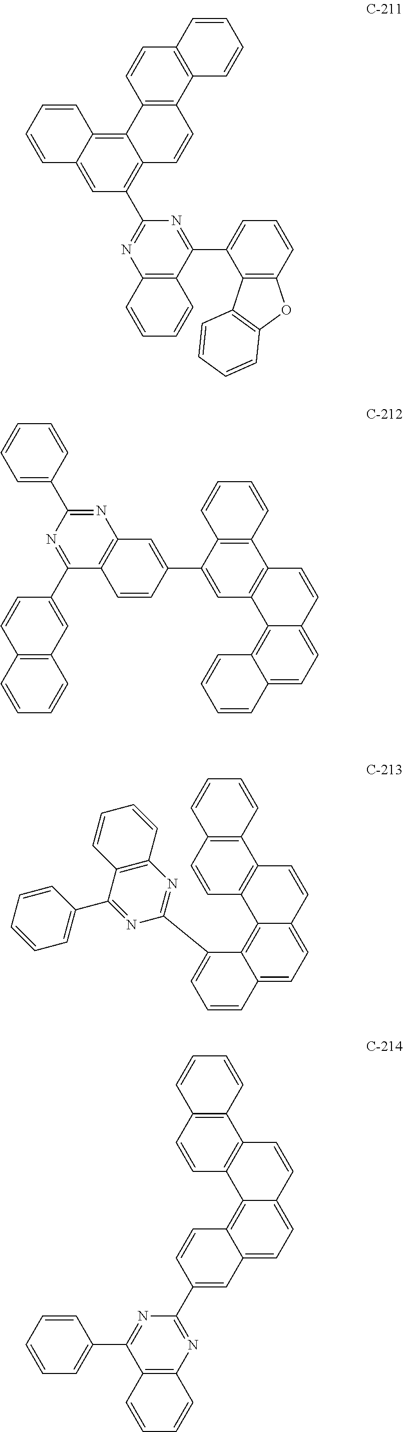 [SMILES: c1ccc2c(c1)ccc1c2ccc2c(-c3nc(-c4cccc5oc6ccccc6c45)c4ccccc4n3)cc3ccccc3c21 ; c1ccc(-c2nc(-c3ccc4ccccc4c3)c3ccc(-c4cc5c(ccc6ccc7ccccc7c65)c5ccccc45)cc3n2)cc1 ; c1ccc(-c2nc(-c3cccc4ccc5ccc6c7ccccc7ccc6c5c34)nc3ccccc23)cc1 ; c1ccc(-c2nc(-c3ccc4c(ccc5ccc6c7ccccc7ccc6c54)c3)nc3ccccc23)cc1]

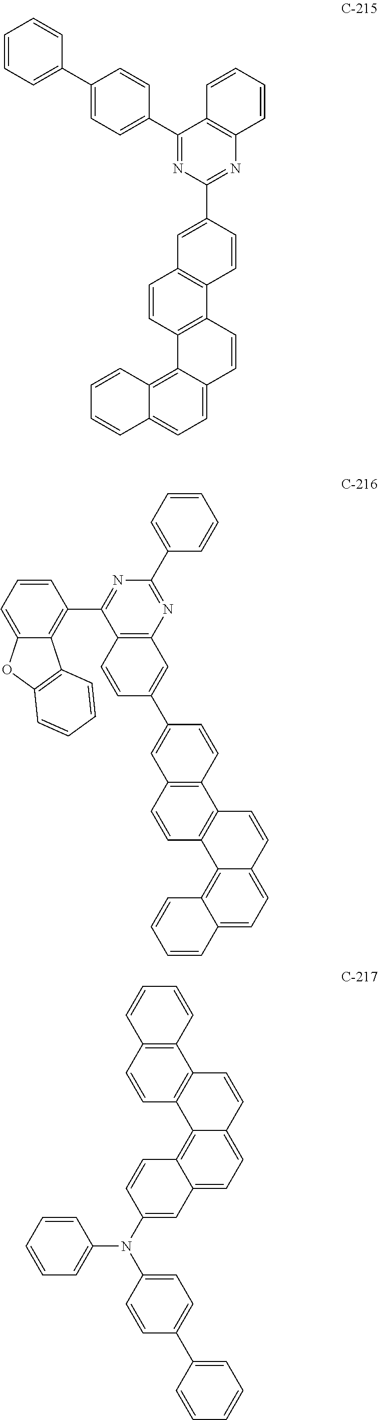

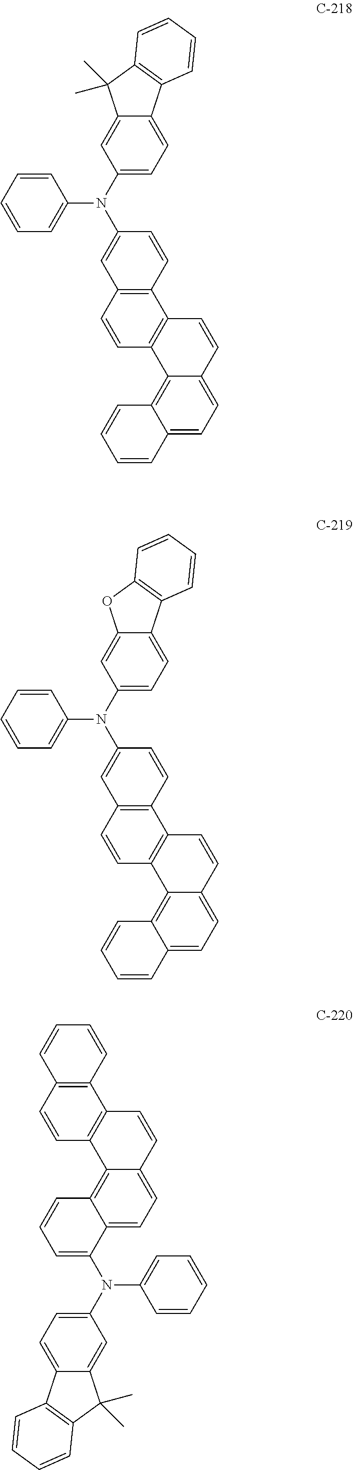 [SMILES: CC1(C)c2ccccc2-c2ccc(N(c3ccccc3)c3ccc4c(ccc5c4ccc4ccc6ccccc6c45)c3)cc21 ; c1ccc(N(c2ccc3c(ccc4c3ccc3ccc5ccccc5c34)c2)c2ccc3c(c2)oc2ccccc23)cc1 ; CC1(C)c2ccccc2-c2ccc(N(c3ccccc3)c3cccc4c3ccc3ccc5c6ccccc6ccc5c34)cc21]

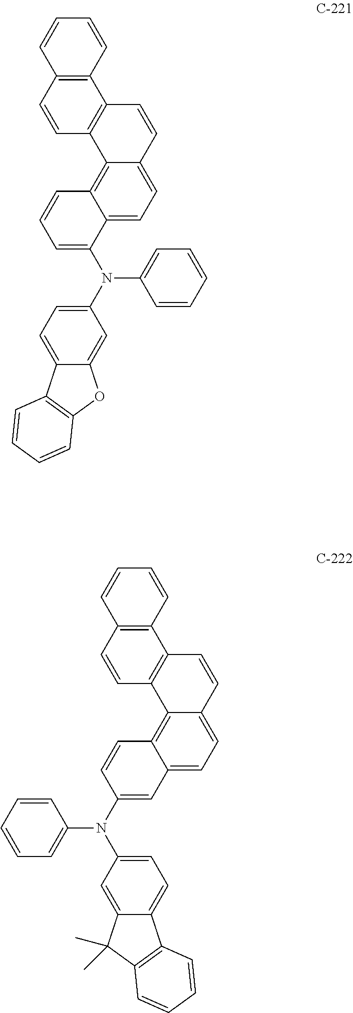 [SMILES: c1ccc(N(c2ccc3c(c2)oc2ccccc23)c2cccc3c2ccc2ccc4c5ccccc5ccc4c23)cc1 ; CC1(C)c2ccccc2-c2ccc(N(c3ccccc3)c3ccc4c(ccc5ccc6c7ccccc7ccc6c54)c3)cc21]

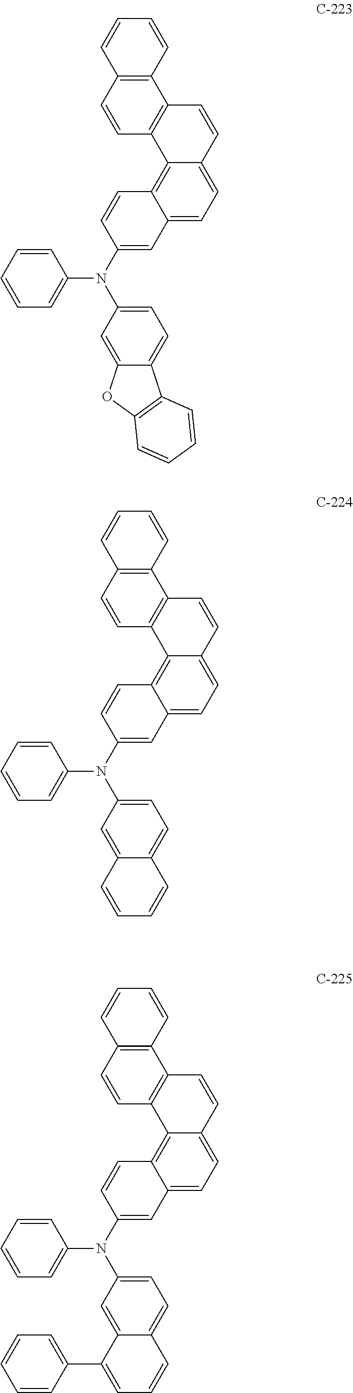 [SMILES: c1ccc(N(c2ccc3c(ccc4ccc5c6ccccc6ccc5c43)c2)c2ccc3c(c2)oc2ccccc23)cc1 ; c1ccc(N(c2ccc3ccccc3c2)c2ccc3c(ccc4ccc5c6ccccc6ccc5c43)c2)cc1 ; c1ccc(-c2cccc3ccc(N(c4ccccc4)c4ccc5c(ccc6ccc7c8ccccc8ccc7c65)c4)cc23)cc1]

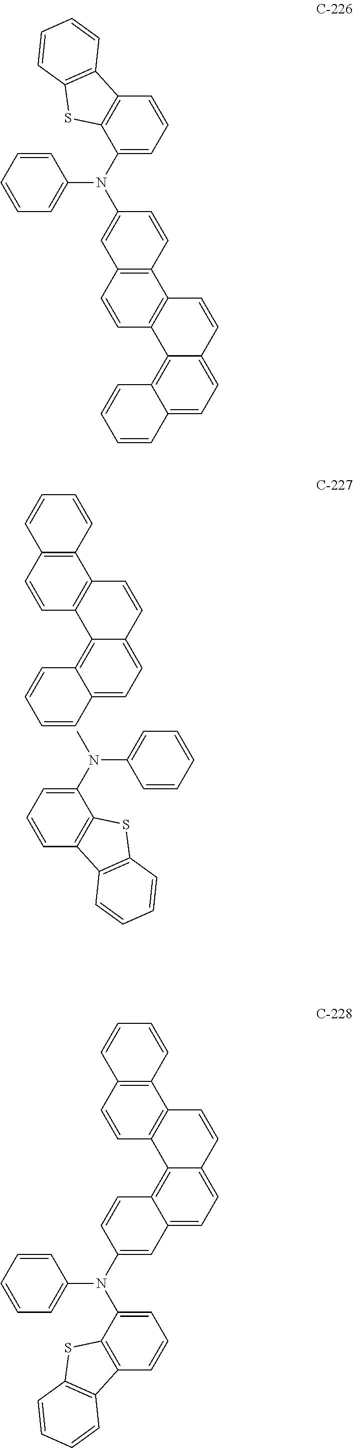

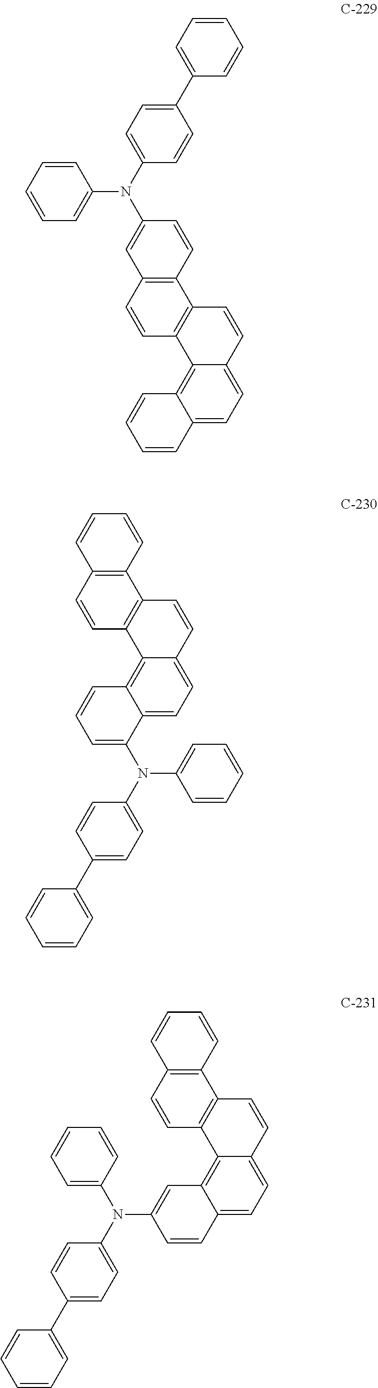 [SMILES: c1ccc(-c2ccc(N(c3ccccc3)c3ccc4c(ccc5c4ccc4ccc6ccccc6c45)c3)cc2)cc1 ; c1ccc(-c2ccc(N(c3ccccc3)c3cccc4c3ccc3ccc5c6ccccc6ccc5c34)cc2)cc1 ; c1ccc(-c2ccc(N(c3ccccc3)c3ccc4ccc5ccc6c7ccccc7ccc6c5c4c3)cc2)cc1]

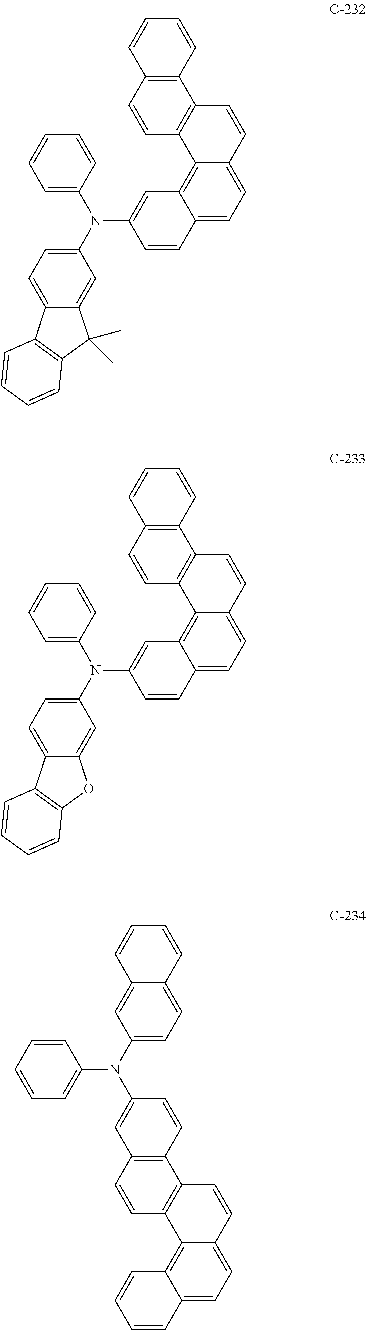 [SMILES: CC1(C)c2ccccc2-c2ccc(N(c3ccccc3)c3ccc4ccc5ccc6c7ccccc7ccc6c5c4c3)cc21 ; c1ccc(N(c2ccc3c(c2)oc2ccccc23)c2ccc3ccc4ccc5c6ccccc6ccc5c4c3c2)cc1 ; c1ccc(N(c2ccc3ccccc3c2)c2ccc3c(ccc4c3ccc3ccc5ccccc5c34)c2)cc1]

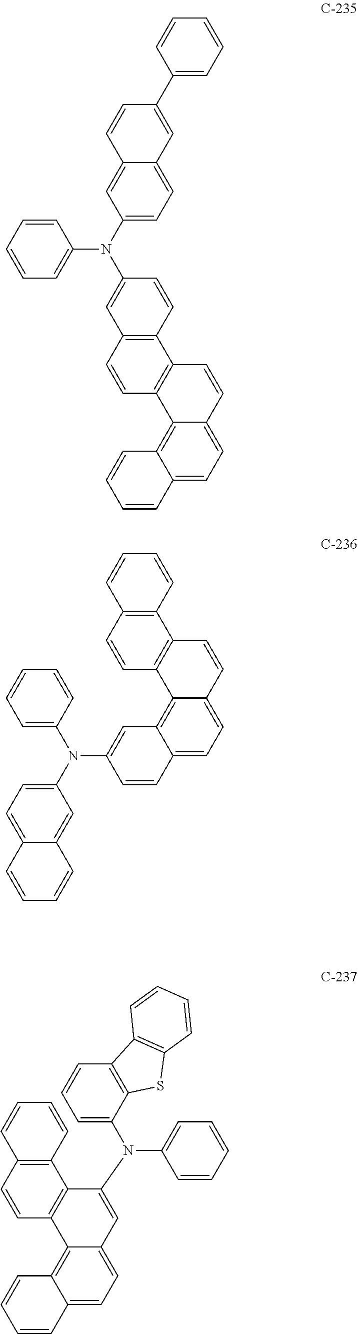

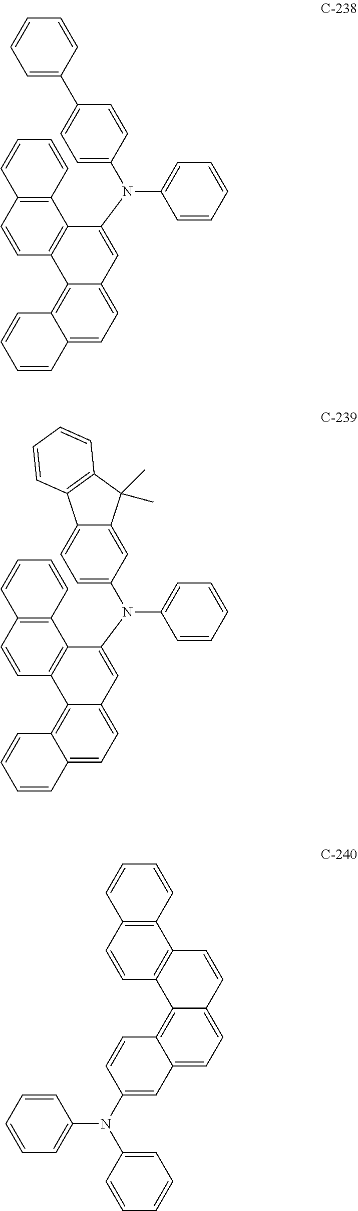 [SMILES: c1ccc(-c2ccc(N(c3ccccc3)c3cc4ccc5ccccc5c4c4ccc5ccccc5c34)cc2)cc1 ; CC1(C)c2ccccc2-c2ccc(N(c3ccccc3)c3cc4ccc5ccccc5c4c4ccc5ccccc5c34)cc21 ; c1ccc(N(c2ccccc2)c2ccc3c(ccc4ccc5c6ccccc6ccc5c43)c2)cc1]

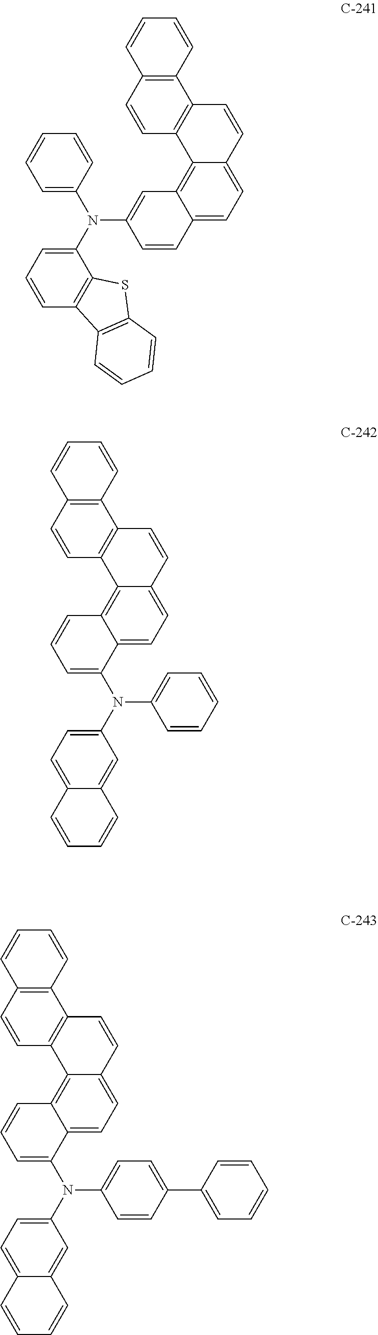 [SMILES: c1ccc(N(c2ccc3ccc4ccc5c6ccccc6ccc5c4c3c2)c2cccc3c2sc2ccccc23)cc1 ; c1ccc(N(c2ccc3ccccc3c2)c2cccc3c2ccc2ccc4c5ccccc5ccc4c23)cc1 ; c1ccc(-c2ccc(N(c3ccc4ccccc4c3)c3cccc4c3ccc3ccc5c6ccccc6ccc5c34)cc2)cc1]

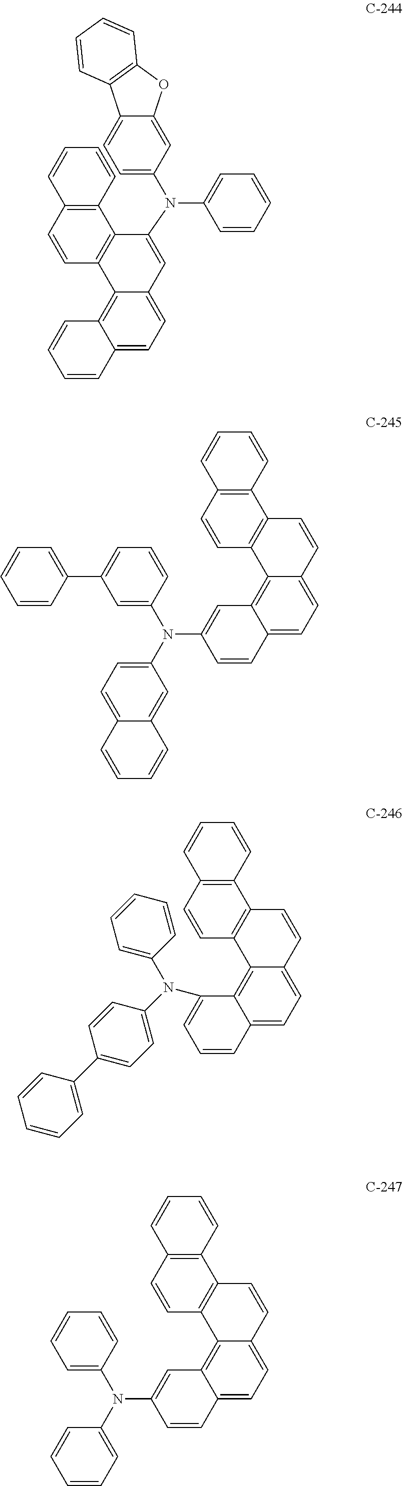 [SMILES: c1ccc(N(c2ccc3c(c2)oc2ccccc23)c2cc3ccc4ccccc4c3c3ccc4ccccc4c23)cc1 ; c1ccc(-c2cccc(N(c3ccc4ccccc4c3)c3ccc4ccc5ccc6c7ccccc7ccc6c5c4c3)c2)cc1 ; c1ccc(-c2ccc(N(c3ccccc3)c3cccc4ccc5ccc6c7ccccc7ccc6c5c34)cc2)cc1 ; c1ccc(N(c2ccccc2)c2ccc3ccc4ccc5c6ccccc6ccc5c4c3c2)cc1]

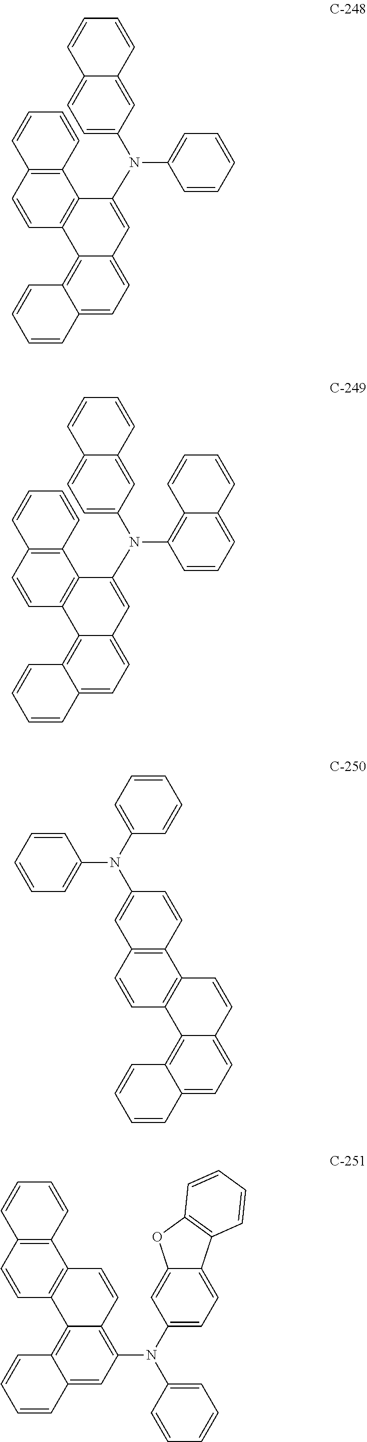 [SMILES: c1ccc(N(c2ccc3ccccc3c2)c2cc3ccc4ccccc4c3c3ccc4ccccc4c23)cc1 ; c1ccc2cc(N(c3cccc4ccccc34)c3cc4ccc5ccccc5c4c4ccc5ccccc5c34)ccc2c1 ; c1ccc(N(c2ccccc2)c2ccc3c(ccc4c3ccc3ccc5ccccc5c34)c2)cc1 ; c1ccc(N(c2ccc3c(c2)oc2ccccc23)c2cc3ccccc3c3c2ccc2c4ccccc4ccc23)cc1]

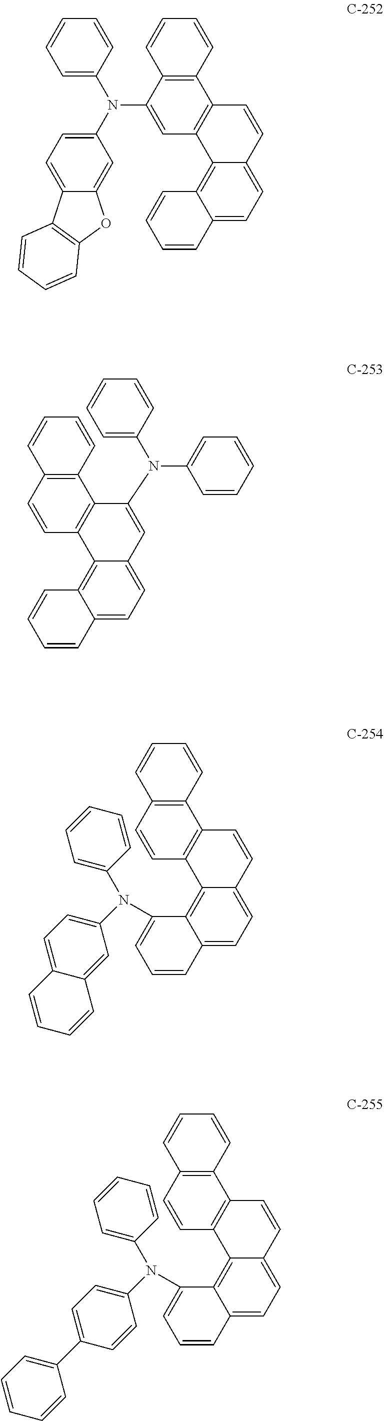

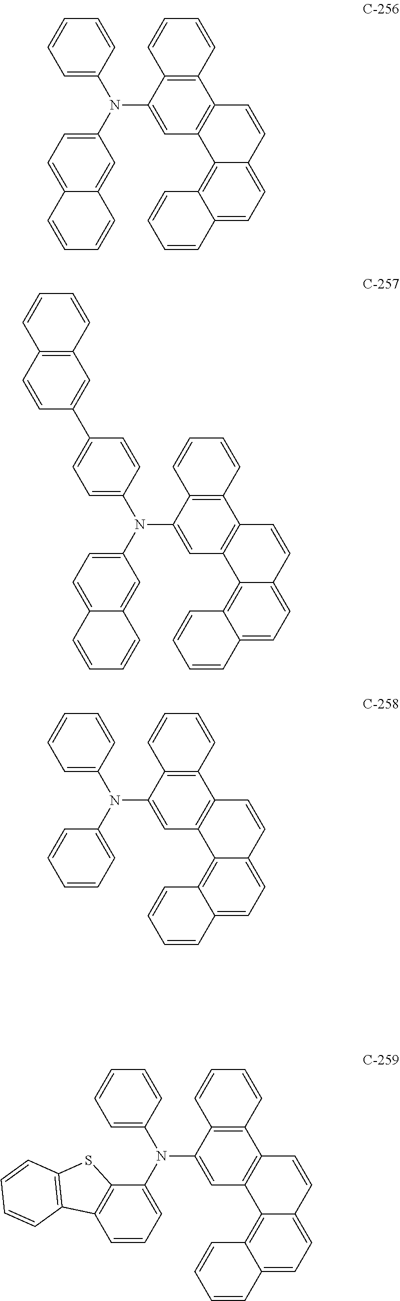

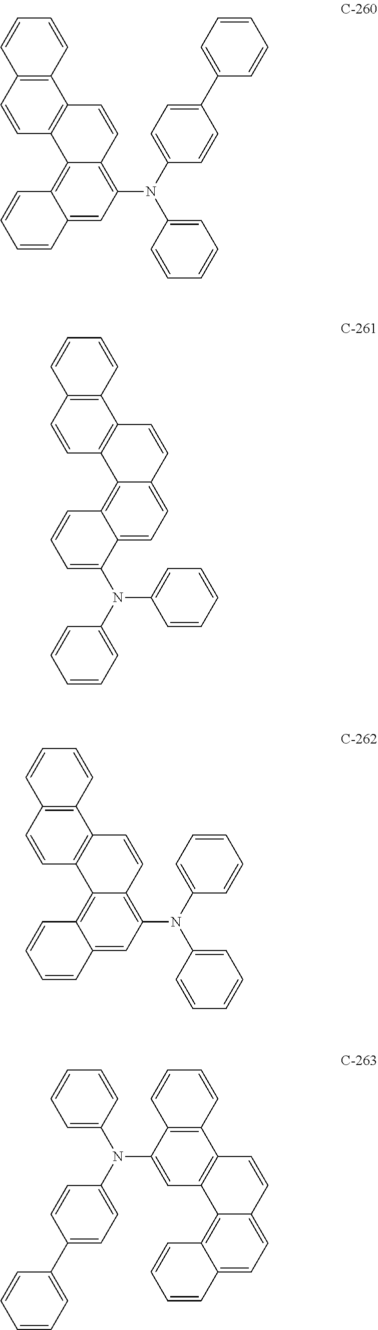 [SMILES: c1ccc(-c2ccc(N(c3ccccc3)c3cc4ccccc4c4c3ccc3c5ccccc5ccc34)cc2)cc1 ; c1ccc(N(c2ccccc2)c2cccc3c2ccc2ccc4c5ccccc5ccc4c23)cc1 ; c1ccc(N(c2ccccc2)c2cc3ccccc3c3c2ccc2c4ccccc4ccc23)cc1 ; c1ccc(-c2ccc(N(c3ccccc3)c3cc4c(ccc5ccc6ccccc6c54)c4ccccc34)cc2)cc1]

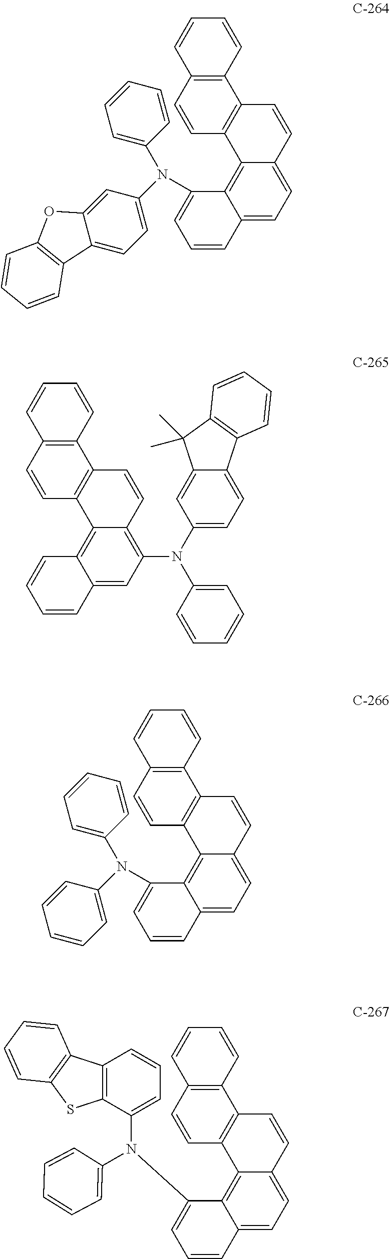

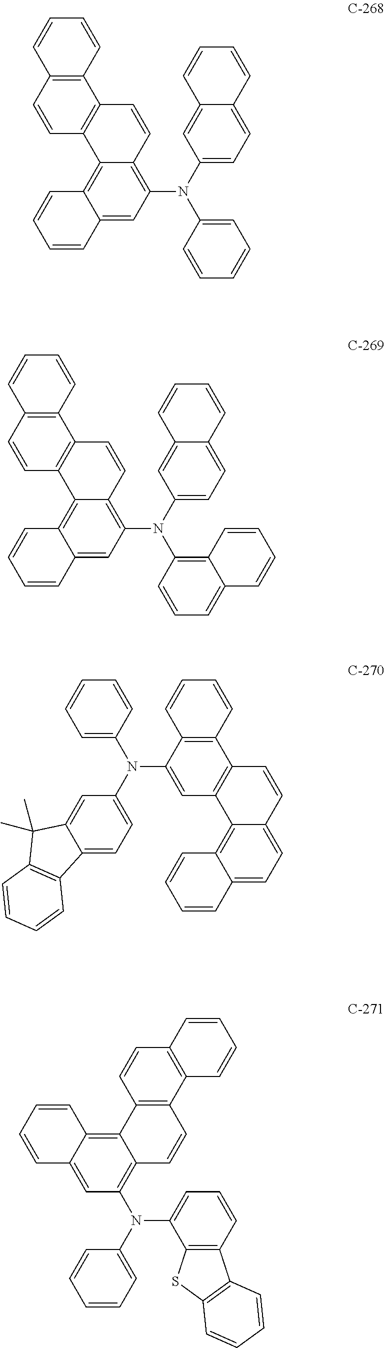 [SMILES: c1ccc(N(c2ccc3ccccc3c2)c2cc3ccccc3c3c2ccc2c4ccccc4ccc23)cc1 ; c1ccc2cc(N(c3cccc4ccccc34)c3cc4ccccc4c4c3ccc3c5ccccc5ccc34)ccc2c1 ; CC1(C)c2ccccc2-c2ccc(N(c3ccccc3)c3cc4c(ccc5ccc6ccccc6c54)c4ccccc34)cc21 ; c1ccc(N(c2cc3ccccc3c3c2ccc2c4ccccc4ccc23)c2cccc3c2sc2ccccc23)cc1]

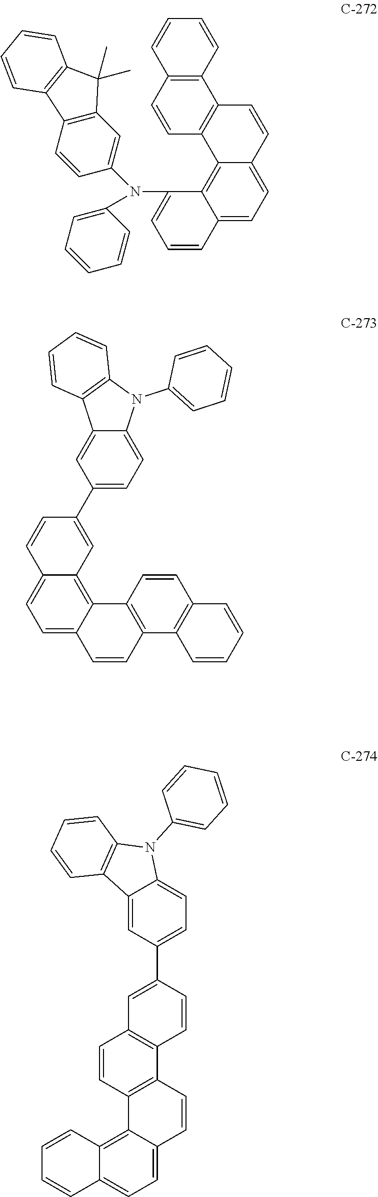 [SMILES: CC1(C)c2ccccc2-c2ccc(N(c3ccccc3)c3cccc4ccc5ccc6c7ccccc7ccc6c5c34)cc21 ; c1ccc(-n2c3ccccc3c3cc(-c4ccc5ccc6ccc7c8ccccc8ccc7c6c5c4)ccc32)cc1 ; c1ccc(-n2c3ccccc3c3cc(-c4ccc5c(ccc6c5ccc5ccc7ccccc7c56)c4)ccc32)cc1]

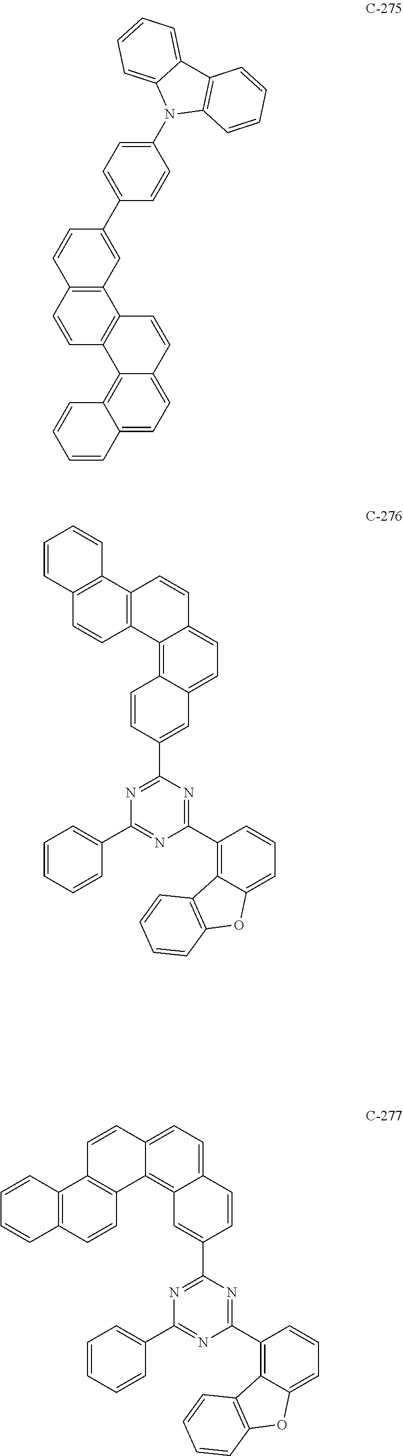 [SMILES: c1ccc2c(c1)ccc1ccc3c4cc(-c5ccc(-n6c7ccccc7c7ccccc76)cc5)ccc4ccc3c12 ; c1ccc(-c2nc(-c3ccc4c(ccc5ccc6c7ccccc7ccc6c54)c3)nc(-c3cccc4oc5ccccc5c34)n2)cc1 ; c1ccc(-c2nc(-c3ccc4ccc5ccc6c7ccccc7ccc6c5c4c3)nc(-c3cccc4oc5ccccc5c34)n2)cc1]

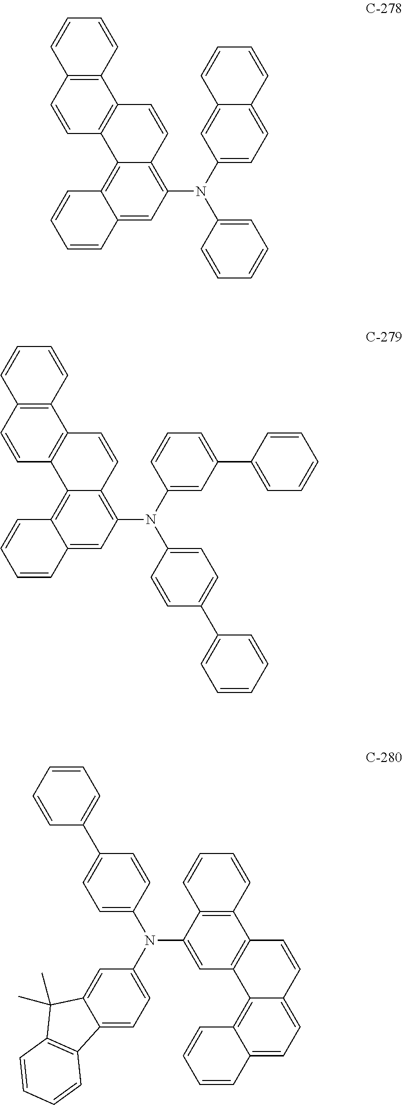 [SMILES: c1ccc(N(c2ccc3ccccc3c2)c2cc3ccccc3c3c2ccc2c4ccccc4ccc23)cc1 ; c1ccc(-c2ccc(N(c3cccc(-c4ccccc4)c3)c3cc4ccccc4c4c3ccc3c5ccccc5ccc34)cc2)cc1 ; CC1(C)c2ccccc2-c2ccc(N(c3ccc(-c4ccccc4)cc3)c3cc4c(ccc5ccc6ccccc6c54)c4ccccc34)cc21]

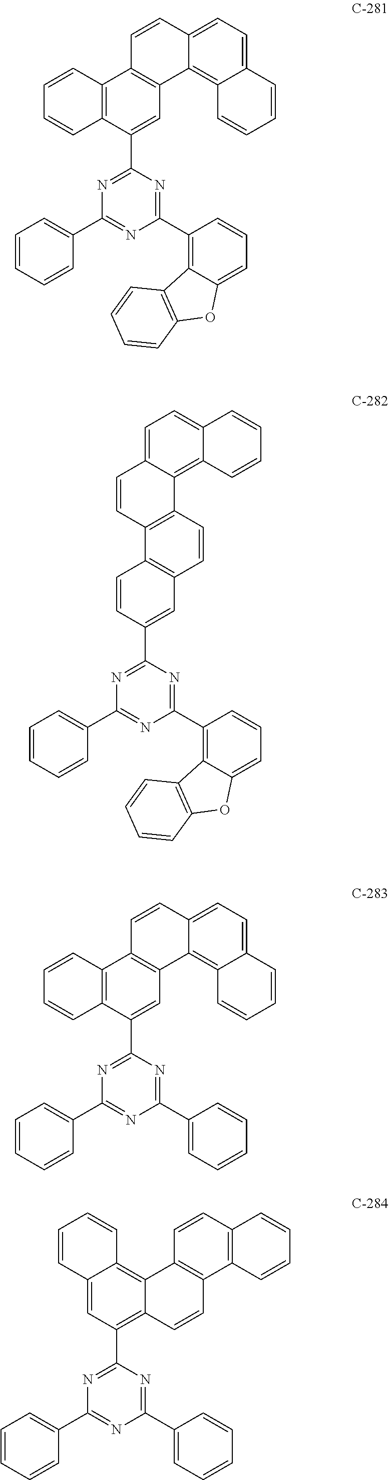

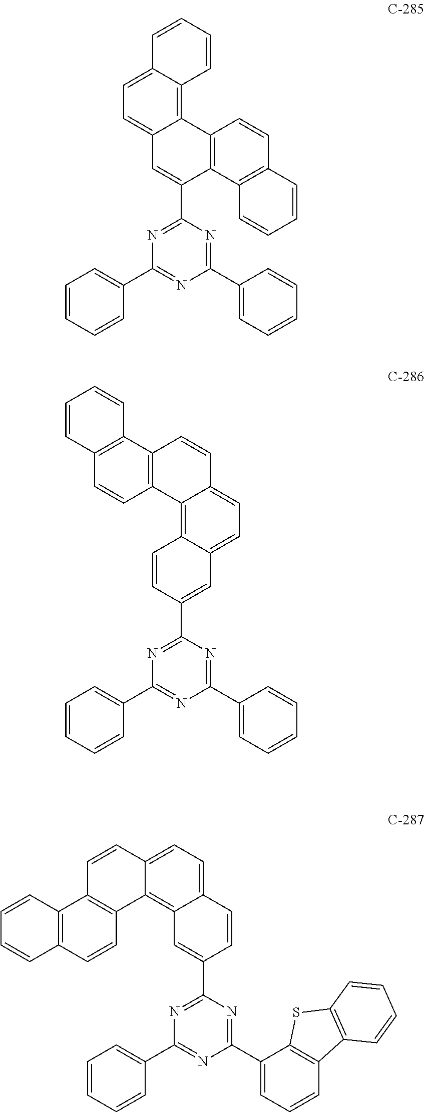 [SMILES: c1ccc(-c2nc(-c3ccccc3)nc(-c3cc4ccc5ccccc5c4c4ccc5ccccc5c34)n2)cc1 ; c1ccc(-c2nc(-c3ccccc3)nc(-c3ccc4c(ccc5ccc6c7ccccc7ccc6c54)c3)n2)cc1 ; c1ccc(-c2nc(-c3ccc4ccc5ccc6c7ccccc7ccc6c5c4c3)nc(-c3cccc4c3sc3ccccc34)n2)cc1]

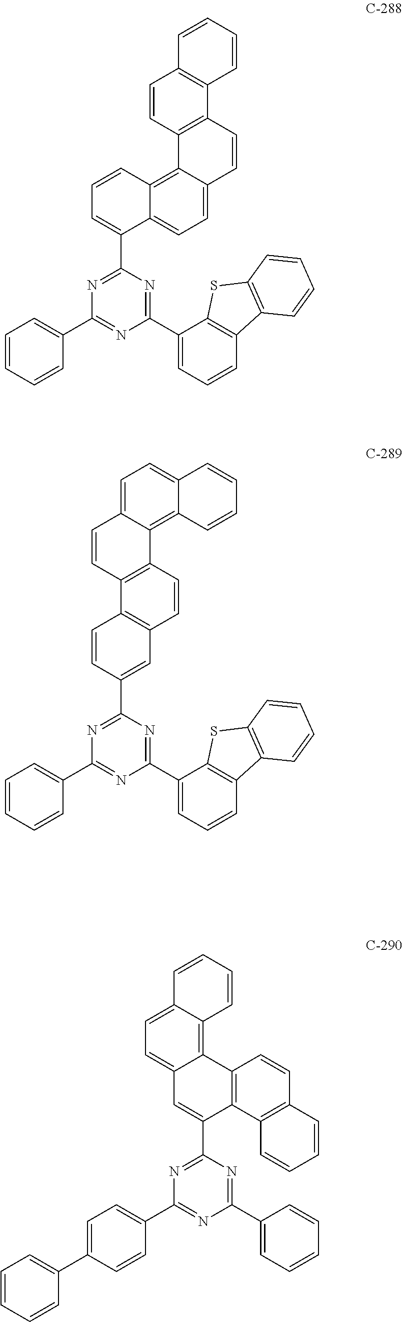 [SMILES: c1ccc(-c2nc(-c3cccc4c3ccc3ccc5c6ccccc6ccc5c34)nc(-c3cccc4c3sc3ccccc34)n2)cc1 ; c1ccc(-c2nc(-c3ccc4c(ccc5c4ccc4ccc6ccccc6c45)c3)nc(-c3cccc4c3sc3ccccc34)n2)cc1 ; c1ccc(-c2ccc(-c3nc(-c4ccccc4)nc(-c4cc5ccc6ccccc6c5c5ccc6ccccc6c45)n3)cc2)cc1]

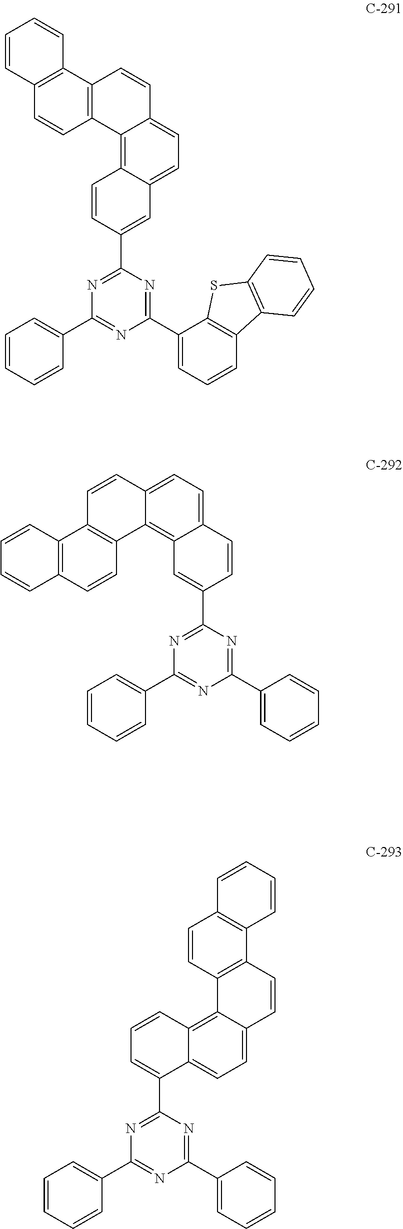 [SMILES: c1ccc(-c2nc(-c3ccc4c(ccc5ccc6c7ccccc7ccc6c54)c3)nc(-c3cccc4c3sc3ccccc34)n2)cc1 ; c1ccc(-c2nc(-c3ccccc3)nc(-c3ccc4ccc5ccc6c7ccccc7ccc6c5c4c3)n2)cc1 ; c1ccc(-c2nc(-c3ccccc3)nc(-c3cccc4c3ccc3ccc5c6ccccc6ccc5c34)n2)cc1]

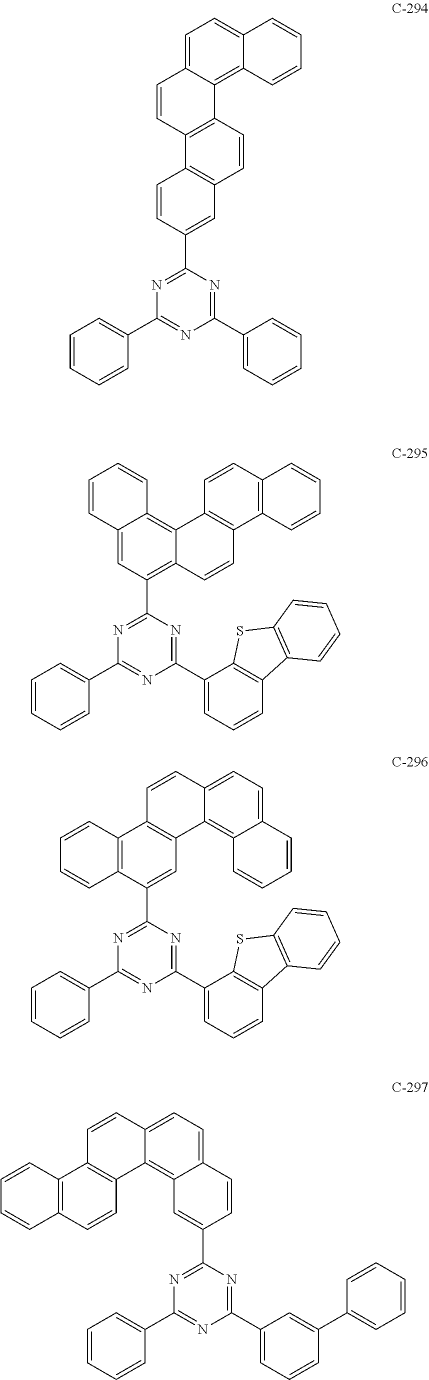

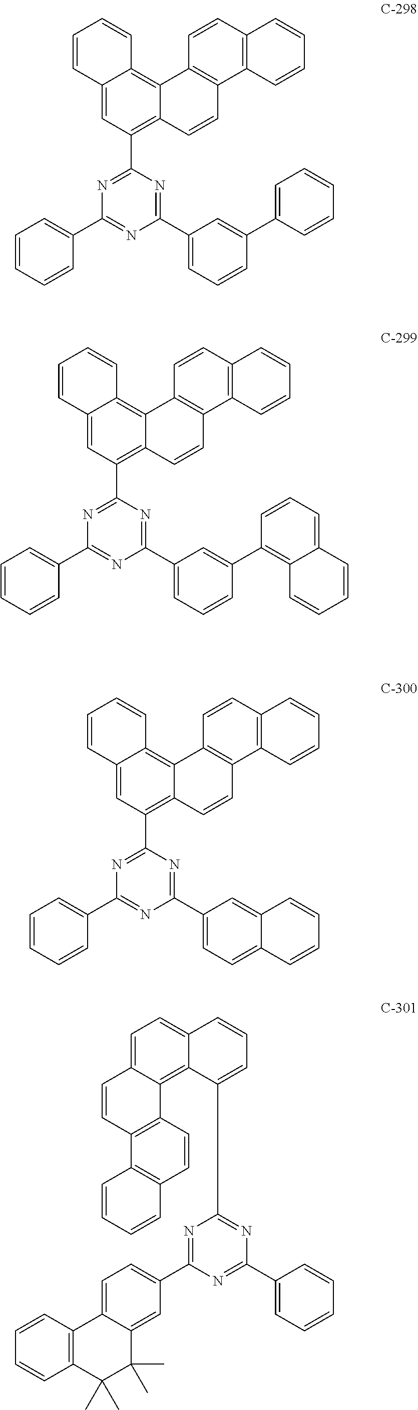 [SMILES: c1ccc(-c2cccc(-c3nc(-c4ccccc4)nc(-c4cc5ccccc5c5c4ccc4c6ccccc6ccc45)n3)c2)cc1 ; c1ccc(-c2nc(-c3cccc(-c4cccc5ccccc45)c3)nc(-c3cc4ccccc4c4c3ccc3c5ccccc5ccc34)n2)cc1 ; c1ccc(-c2nc(-c3ccc4ccccc4c3)nc(-c3cc4ccccc4c4c3ccc3c5ccccc5ccc34)n2)cc1 ; CC1(C)c2ccccc2-c2ccc(-c3nc(-c4ccccc4)nc(-c4cccc5ccc6ccc7c8ccccc8ccc7c6c45)n3)cc2C1(C)C]

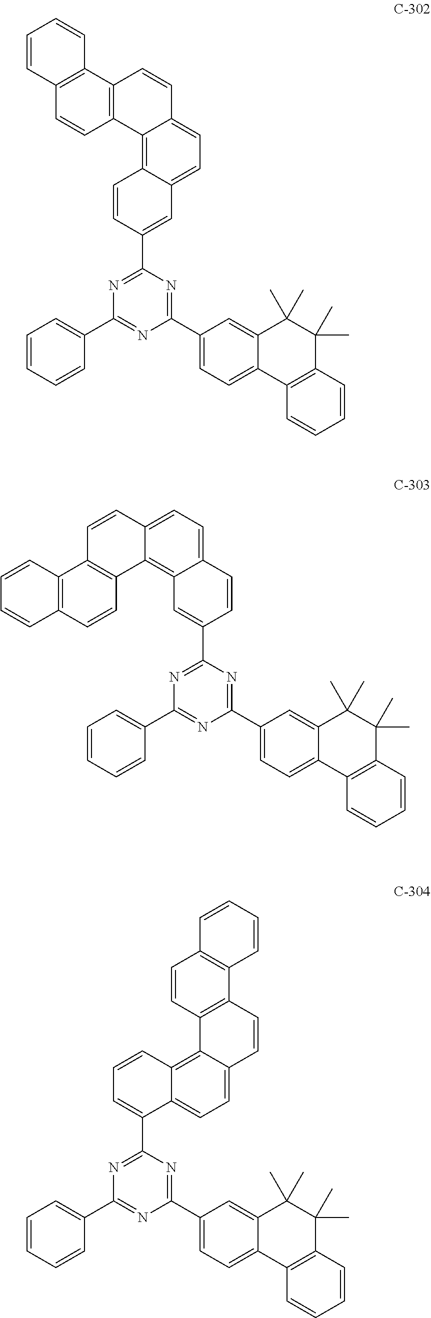 [SMILES: CC1(C)c2ccccc2-c2ccc(-c3nc(-c4ccccc4)nc(-c4ccc5c(ccc6ccc7c8ccccc8ccc7c65)c4)n3)cc2C1(C)C ; CC1(C)c2ccccc2-c2ccc(-c3nc(-c4ccccc4)nc(-c4ccc5ccc6ccc7c8ccccc8ccc7c6c5c4)n3)cc2C1(C)C ; CC1(C)c2ccccc2-c2ccc(-c3nc(-c4ccccc4)nc(-c4cccc5c4ccc4ccc6c7ccccc7ccc6c45)n3)cc2C1(C)C]

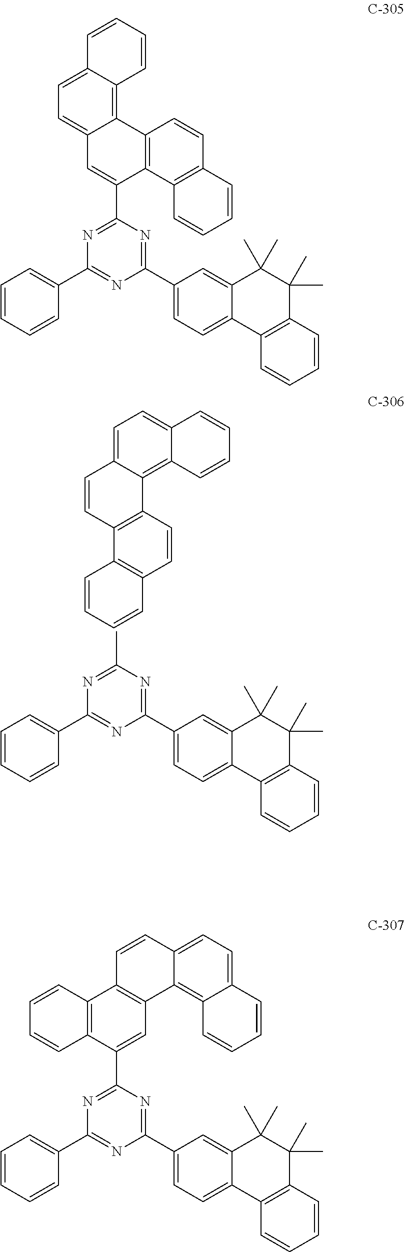

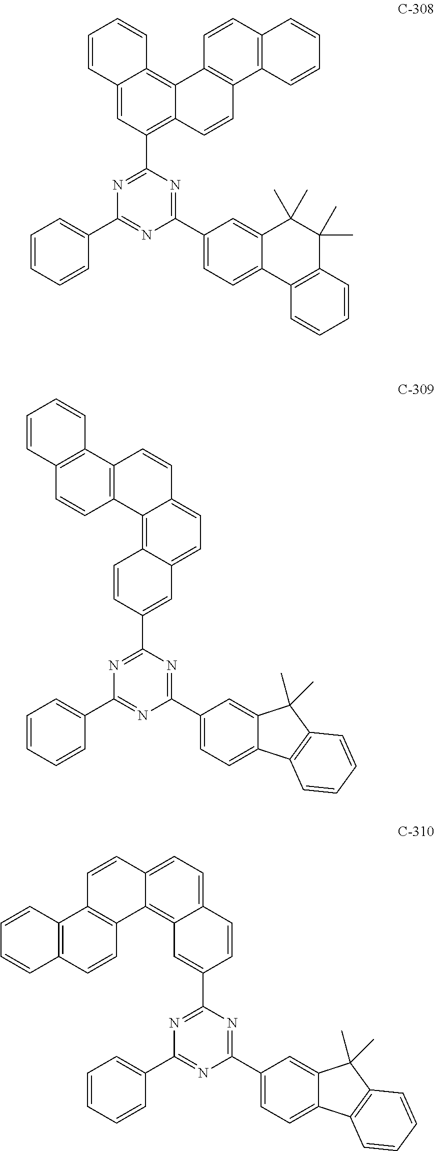 [SMILES: CC1(C)c2ccccc2-c2ccc(-c3nc(-c4ccccc4)nc(-c4cc5ccccc5c5c4ccc4c6ccccc6ccc45)n3)cc2C1(C)C ; CC1(C)c2ccccc2-c2ccc(-c3nc(-c4ccccc4)nc(-c4ccc5c(ccc6ccc7c8ccccc8ccc7c65)c4)n3)cc21 ; CC1(C)c2ccccc2-c2ccc(-c3nc(-c4ccccc4)nc(-c4ccc5ccc6ccc7c8ccccc8ccc7c6c5c4)n3)cc21]

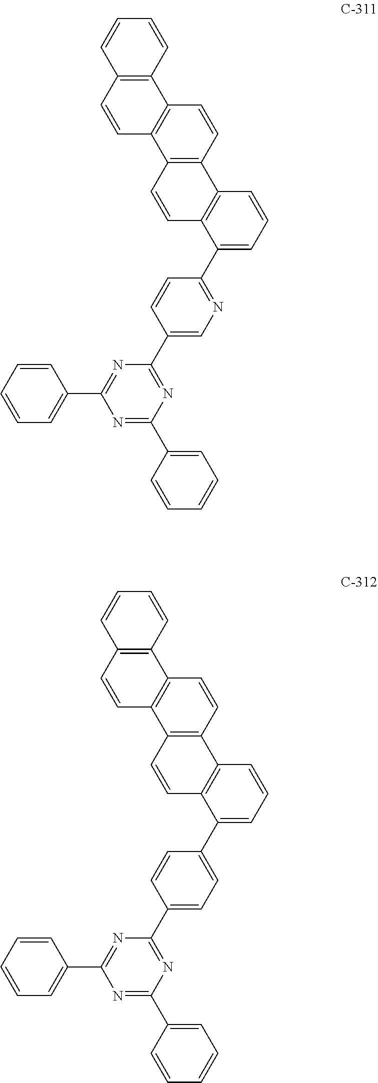 [SMILES: c1ccc(-c2nc(-c3ccccc3)nc(-c3ccc(-c4cccc5c4ccc4c5ccc5c6ccccc6ccc54)nc3)n2)cc1 ; c1ccc(-c2nc(-c3ccccc3)nc(-c3ccc(-c4cccc5c4ccc4c5ccc5c6ccccc6ccc54)cc3)n2)cc1]

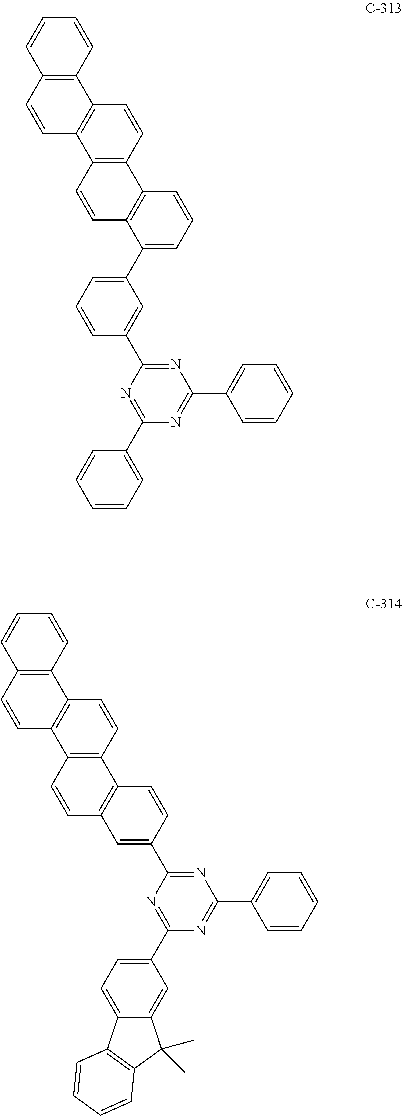 [SMILES: c1ccc(-c2nc(-c3ccccc3)nc(-c3cccc(-c4cccc5c4ccc4c5ccc5c6ccccc6ccc54)c3)n2)cc1 ; CC1(C)c2ccccc2-c2ccc(-c3nc(-c4ccccc4)nc(-c4ccc5c(ccc6c5ccc5c7ccccc7ccc56)c4)n3)cc21]

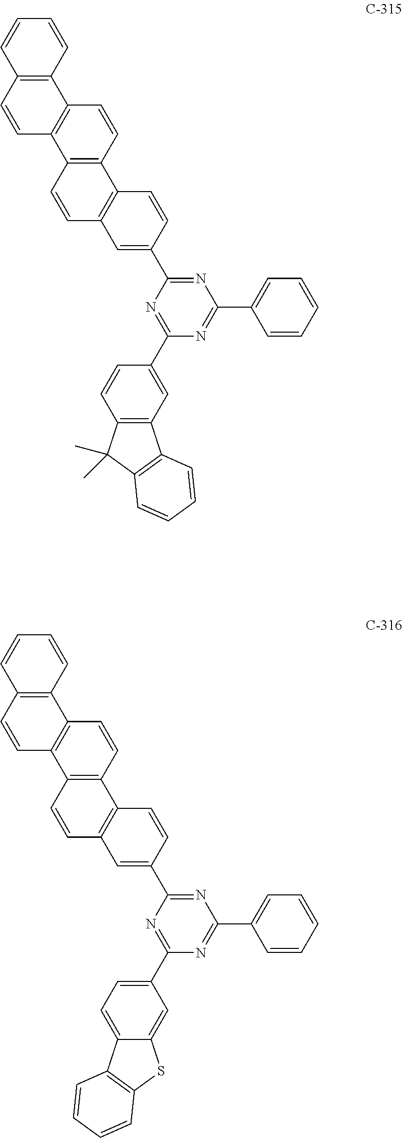 [SMILES: CC1(C)c2ccccc2-c2cc(-c3nc(-c4ccccc4)nc(-c4ccc5c(ccc6c5ccc5c7ccccc7ccc56)c4)n3)ccc21 ; c1ccc(-c2nc(-c3ccc4c(ccc5c4ccc4c6ccccc6ccc45)c3)nc(-c3ccc4c(c3)sc3ccccc34)n2)cc1]

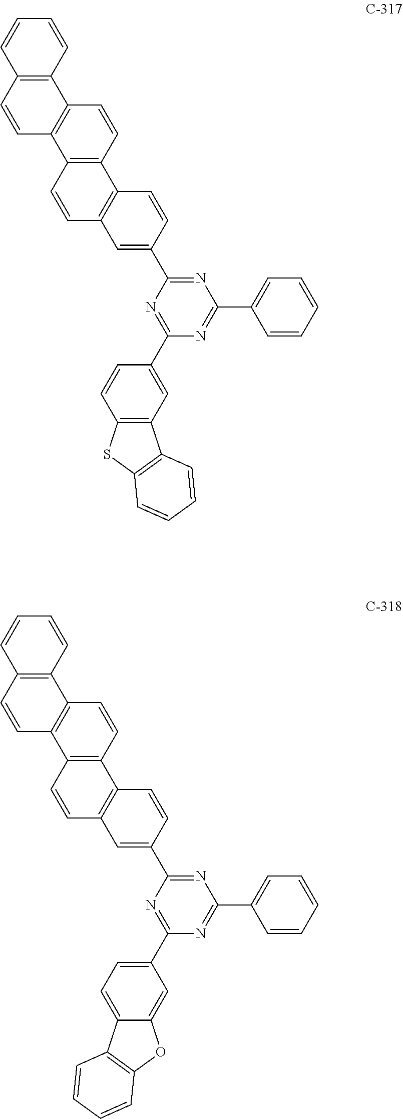 [SMILES: c1ccc(-c2nc(-c3ccc4c(ccc5c4ccc4c6ccccc6ccc45)c3)nc(-c3ccc4sc5ccccc5c4c3)n2)cc1 ; c1ccc(-c2nc(-c3ccc4c(ccc5c4ccc4c6ccccc6ccc45)c3)nc(-c3ccc4c(c3)oc3ccccc34)n2)cc1]

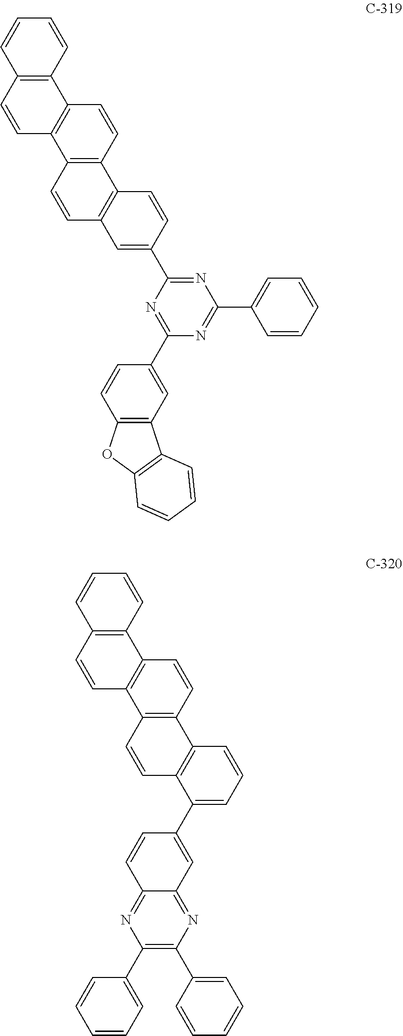 [SMILES: c1ccc(-c2nc(-c3ccc4c(ccc5c4ccc4c6ccccc6ccc45)c3)nc(-c3ccc4oc5ccccc5c4c3)n2)cc1 ; c1ccc(-c2nc3ccc(-c4cccc5c4ccc4c5ccc5c6ccccc6ccc54)cc3nc2-c2ccccc2)cc1]

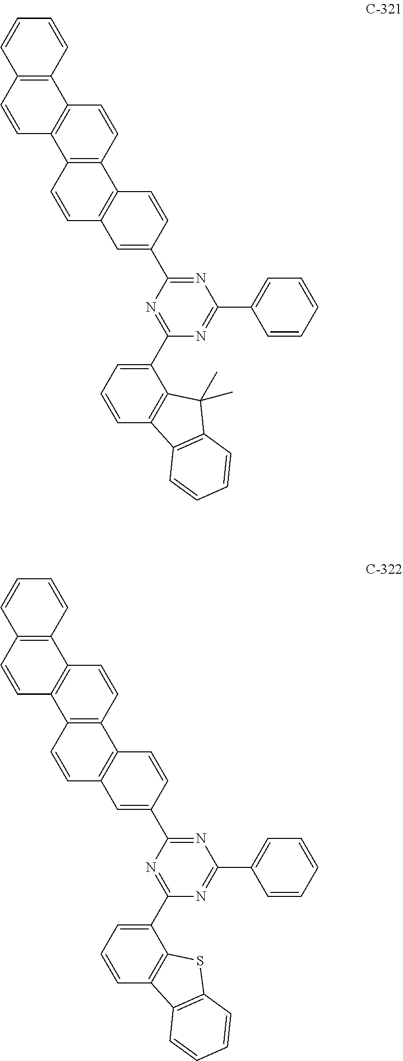 [SMILES: CC1(C)c2ccccc2-c2cccc(-c3nc(-c4ccccc4)nc(-c4ccc5c(ccc6c5ccc5c7ccccc7ccc56)c4)n3)c21 ; c1ccc(-c2nc(-c3ccc4c(ccc5c4ccc4c6ccccc6ccc45)c3)nc(-c3cccc4c3sc3ccccc34)n2)cc1]

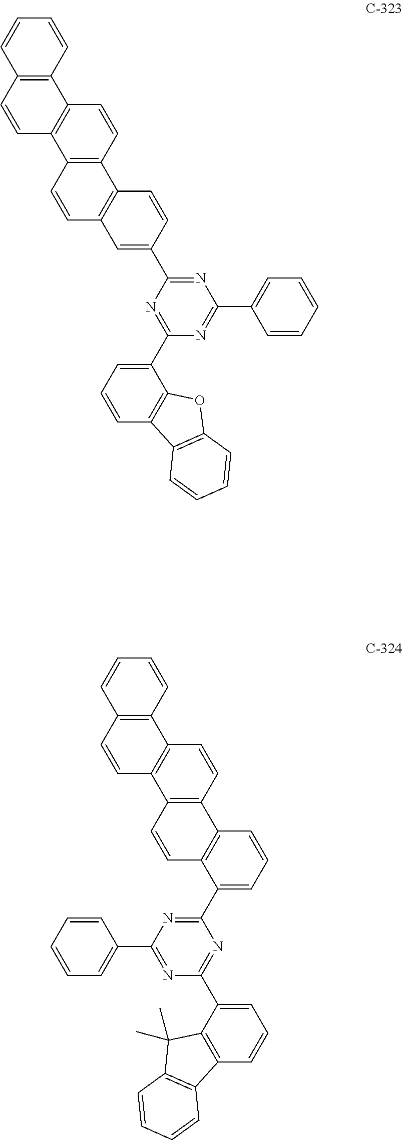 [SMILES: c1ccc(-c2nc(-c3ccc4c(ccc5c4ccc4c6ccccc6ccc45)c3)nc(-c3cccc4c3oc3ccccc34)n2)cc1 ; CC1(C)c2ccccc2-c2cccc(-c3nc(-c4ccccc4)nc(-c4cccc5c4ccc4c5ccc5c6ccccc6ccc54)n3)c21]

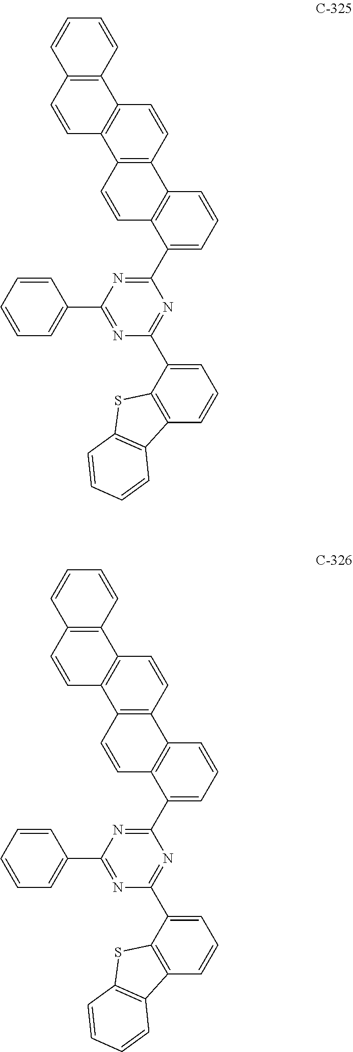 [SMILES: c1ccc(-c2nc(-c3cccc4c3ccc3c4ccc4c5ccccc5ccc43)nc(-c3cccc4c3sc3ccccc34)n2)cc1 ; c1ccc(-c2nc(-c3cccc4c3ccc3c4ccc4c5ccccc5ccc43)nc(-c3cccc4c3sc3ccccc34)n2)cc1]

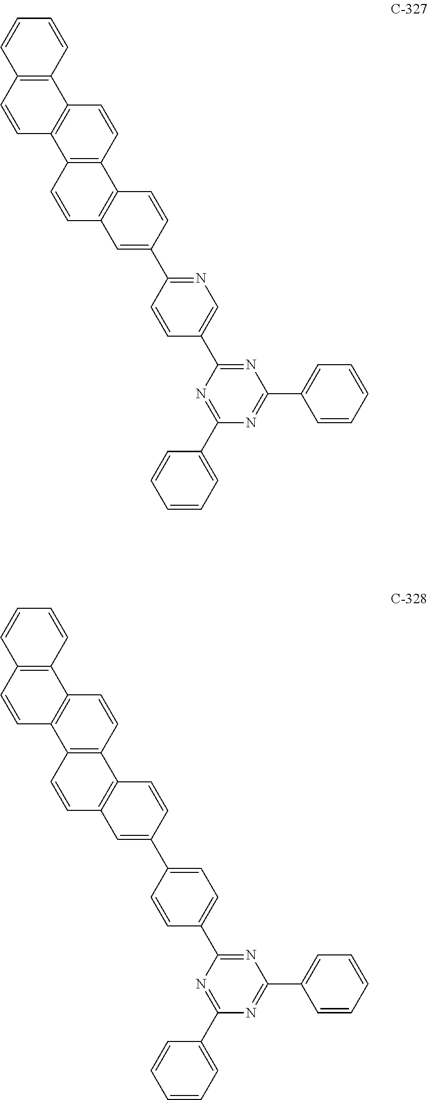 [SMILES: c1ccc(-c2nc(-c3ccccc3)nc(-c3ccc(-c4ccc5c(ccc6c5ccc5c7ccccc7ccc56)c4)nc3)n2)cc1 ; c1ccc(-c2nc(-c3ccccc3)nc(-c3ccc(-c4ccc5c(ccc6c5ccc5c7ccccc7ccc56)c4)cc3)n2)cc1]

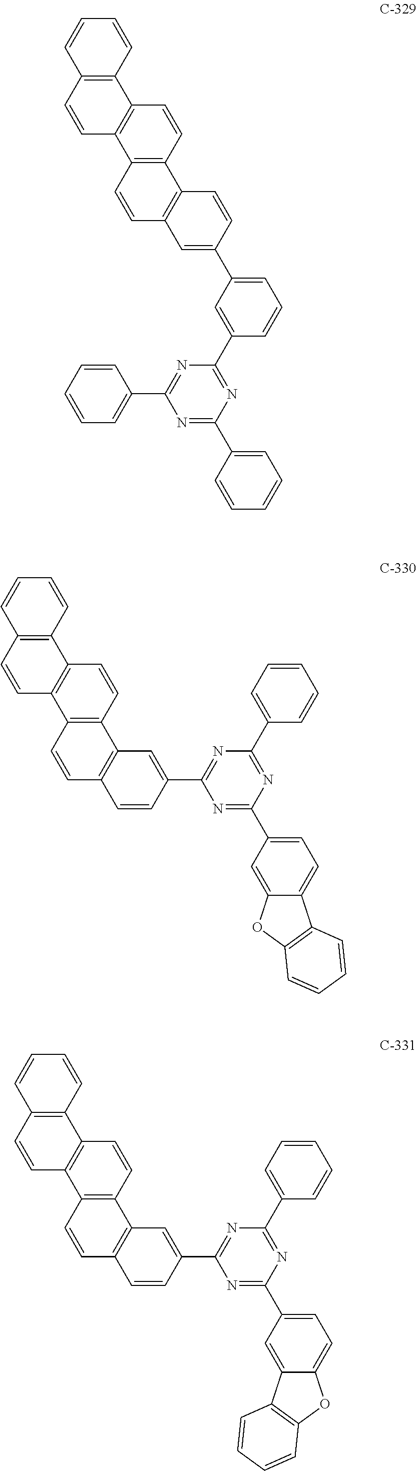 [SMILES: c1ccc(-c2nc(-c3ccccc3)nc(-c3cccc(-c4ccc5c(ccc6c5ccc5c7ccccc7ccc56)c4)c3)n2)cc1 ; c1ccc(-c2nc(-c3ccc4c(c3)oc3ccccc34)nc(-c3ccc4ccc5c(ccc6c7ccccc7ccc65)c4c3)n2)cc1 ; c1ccc(-c2nc(-c3ccc4ccc5c(ccc6c7ccccc7ccc65)c4c3)nc(-c3ccc4oc5ccccc5c4c3)n2)cc1]

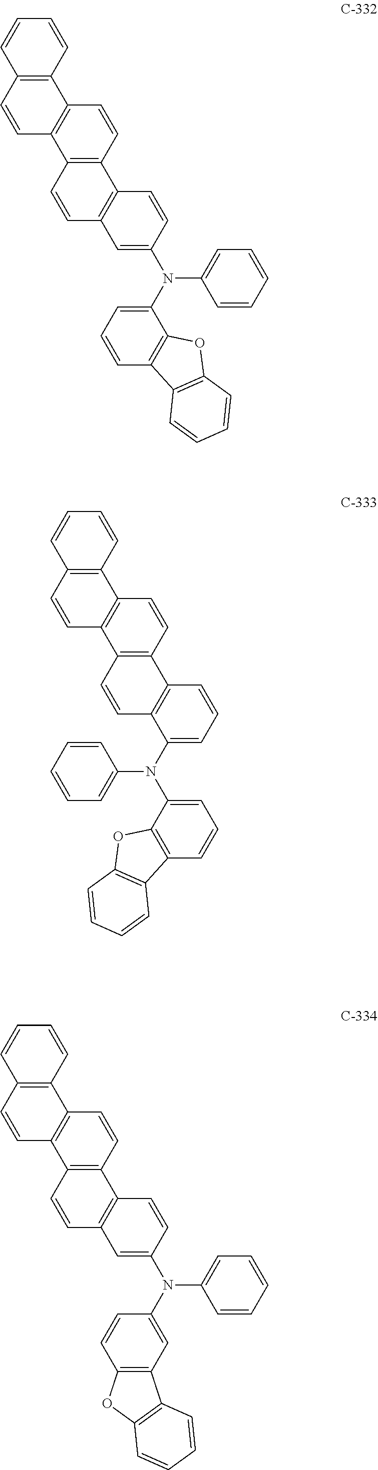 [SMILES: c1ccc(N(c2ccc3c(ccc4c3ccc3c5ccccc5ccc34)c2)c2cccc3c2oc2ccccc23)cc1 ; c1ccc(N(c2cccc3c2ccc2c3ccc3c4ccccc4ccc32)c2cccc3c2oc2ccccc23)cc1 ; c1ccc(N(c2ccc3c(ccc4c3ccc3c5ccccc5ccc34)c2)c2ccc3oc4ccccc4c3c2)cc1]

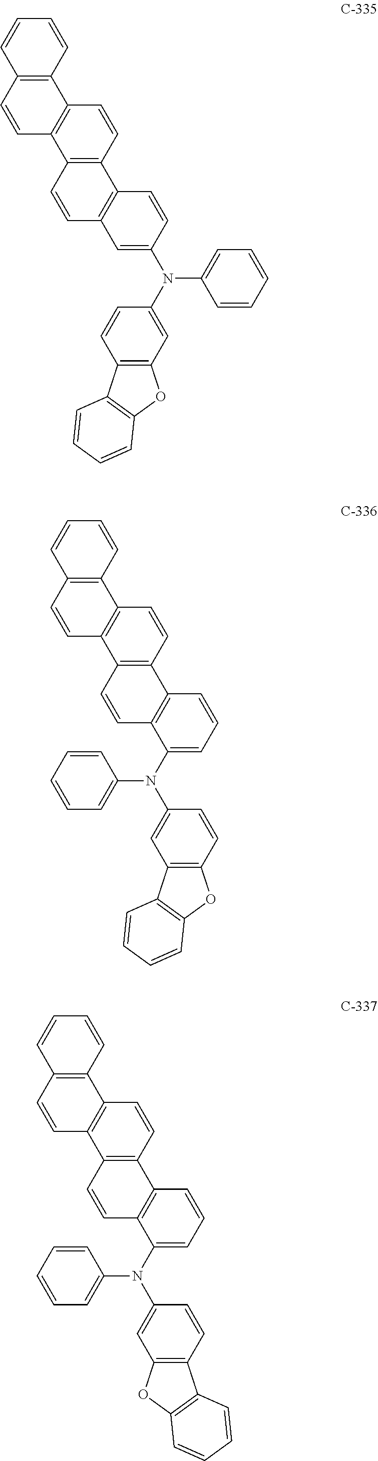 [SMILES: c1ccc(N(c2ccc3c(ccc4c3ccc3c5ccccc5ccc34)c2)c2ccc3c(c2)oc2ccccc23)cc1 ; c1ccc(N(c2ccc3oc4ccccc4c3c2)c2cccc3c2ccc2c3ccc3c4ccccc4ccc32)cc1 ; c1ccc(N(c2ccc3c(c2)oc2ccccc23)c2cccc3c2ccc2c3ccc3c4ccccc4ccc32)cc1]

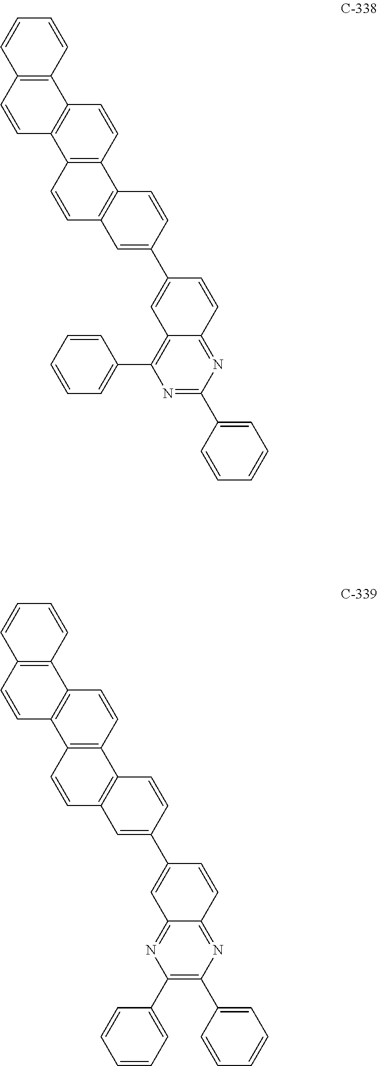 [SMILES: c1ccc(-c2nc(-c3ccccc3)c3cc(-c4ccc5c(ccc6c5ccc5c7ccccc7ccc56)c4)ccc3n2)cc1 ; c1ccc(-c2nc3ccc(-c4ccc5c(ccc6c5ccc5c7ccccc7ccc56)c4)cc3nc2-c2ccccc2)cc1]

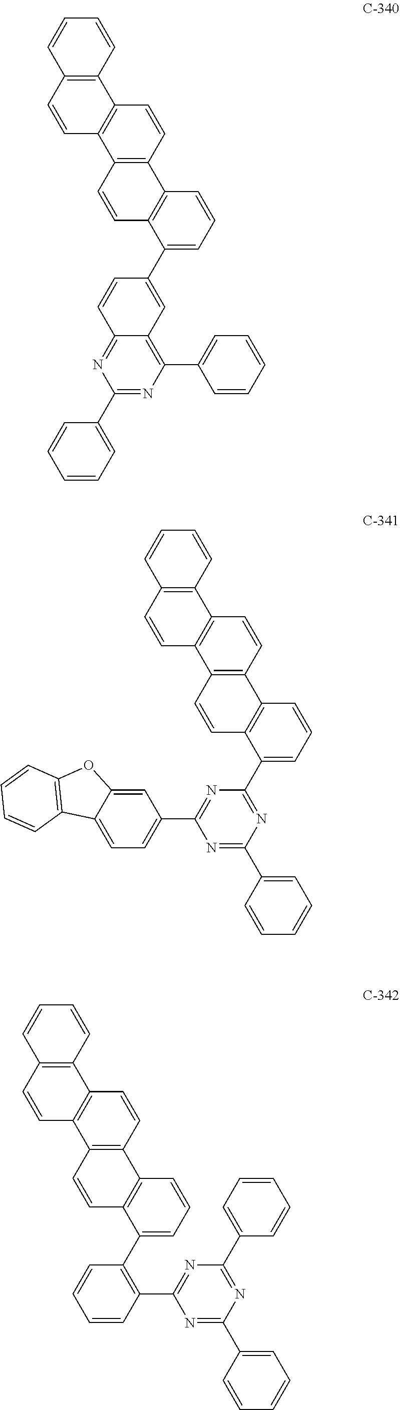 [SMILES: c1ccc(-c2nc(-c3ccccc3)c3cc(-c4cccc5c4ccc4c5ccc5c6ccccc6ccc54)ccc3n2)cc1 ; c1ccc(-c2nc(-c3ccc4c(c3)oc3ccccc34)nc(-c3cccc4c3ccc3c4ccc4c5ccccc5ccc43)n2)cc1 ; c1ccc(-c2nc(-c3ccccc3)nc(-c3ccccc3-c3cccc4c3ccc3c4ccc4c5ccccc5ccc43)n2)cc1]

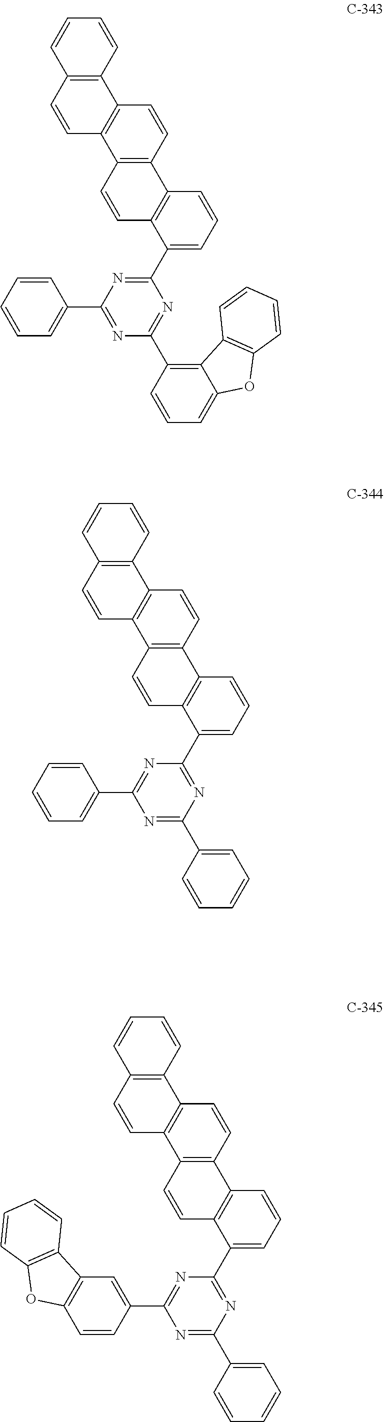 [SMILES: c1ccc(-c2nc(-c3cccc4c3ccc3c4ccc4c5ccccc5ccc43)nc(-c3cccc4oc5ccccc5c34)n2)cc1 ; c1ccc(-c2nc(-c3ccccc3)nc(-c3cccc4c3ccc3c4ccc4c5ccccc5ccc43)n2)cc1 ; c1ccc(-c2nc(-c3ccc4oc5ccccc5c4c3)nc(-c3cccc4c3ccc3c4ccc4c5ccccc5ccc43)n2)cc1]

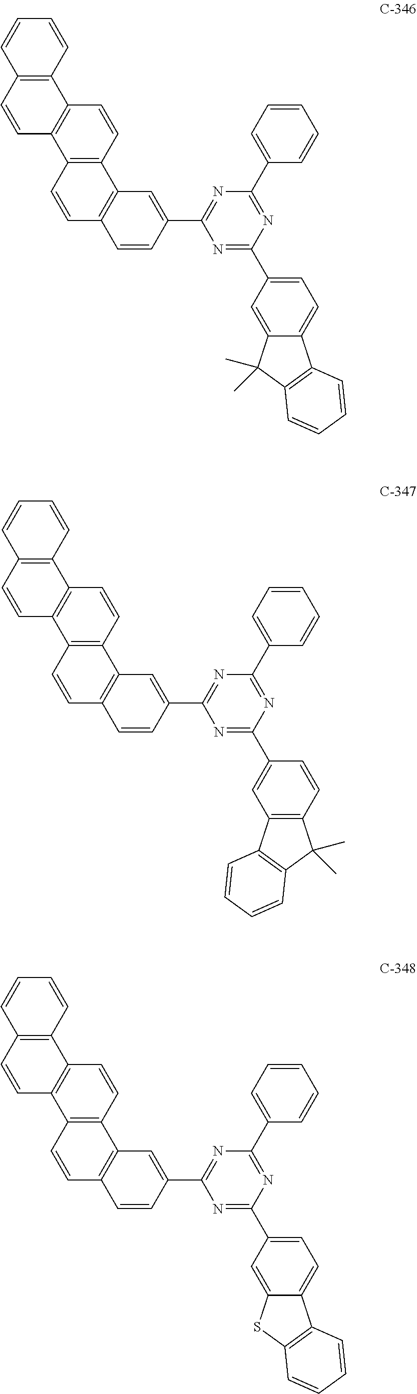 [SMILES: CC1(C)c2ccccc2-c2ccc(-c3nc(-c4ccccc4)nc(-c4ccc5ccc6c(ccc7c8ccccc8ccc76)c5c4)n3)cc21 ; CC1(C)c2ccccc2-c2cc(-c3nc(-c4ccccc4)nc(-c4ccc5ccc6c(ccc7c8ccccc8ccc76)c5c4)n3)ccc21 ; c1ccc(-c2nc(-c3ccc4c(c3)sc3ccccc34)nc(-c3ccc4ccc5c(ccc6c7ccccc7ccc65)c4c3)n2)cc1]

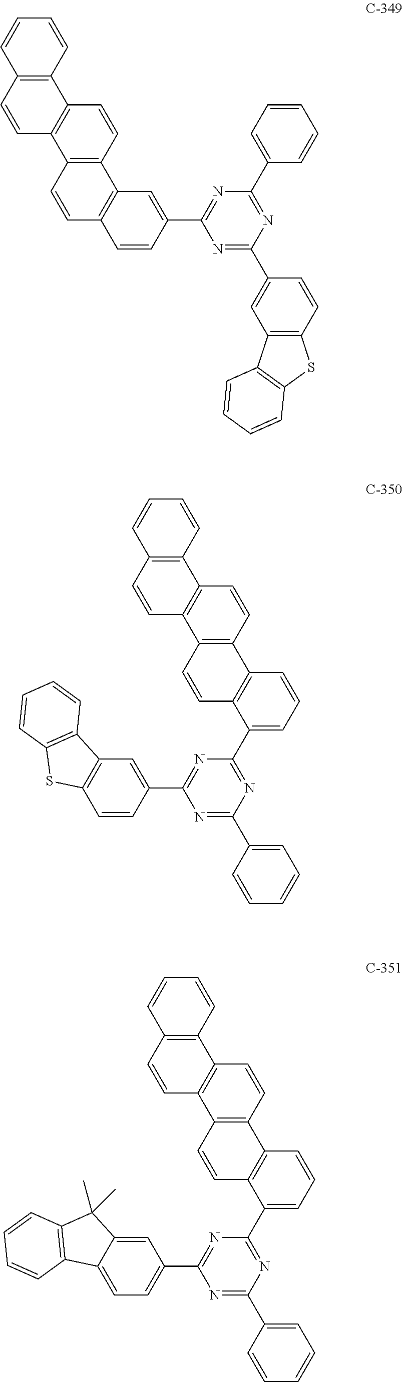 [SMILES: c1ccc(-c2nc(-c3ccc4ccc5c(ccc6c7ccccc7ccc65)c4c3)nc(-c3ccc4sc5ccccc5c4c3)n2)cc1 ; c1ccc(-c2nc(-c3ccc4sc5ccccc5c4c3)nc(-c3cccc4c3ccc3c4ccc4c5ccccc5ccc43)n2)cc1 ; CC1(C)c2ccccc2-c2ccc(-c3nc(-c4ccccc4)nc(-c4cccc5c4ccc4c5ccc5c6ccccc6ccc54)n3)cc21]

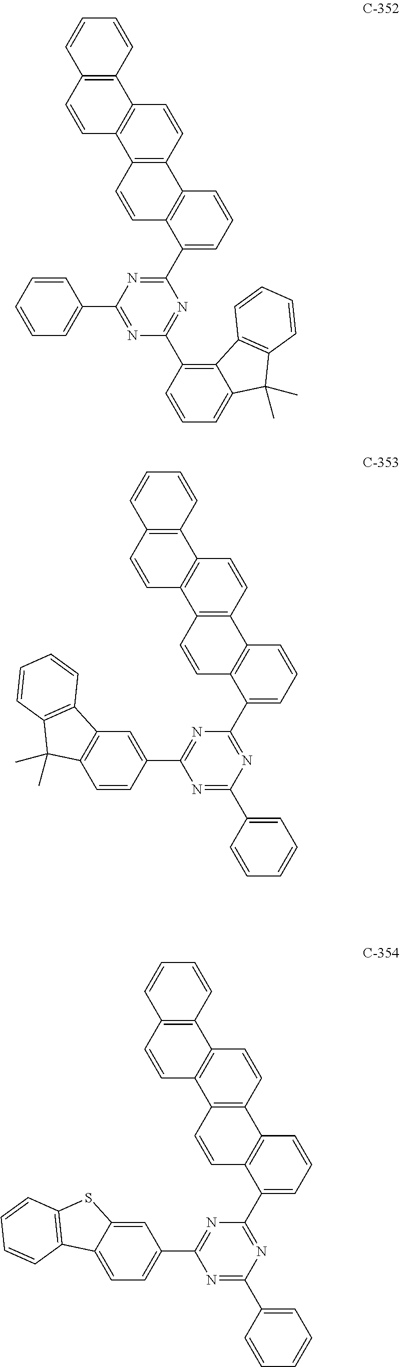

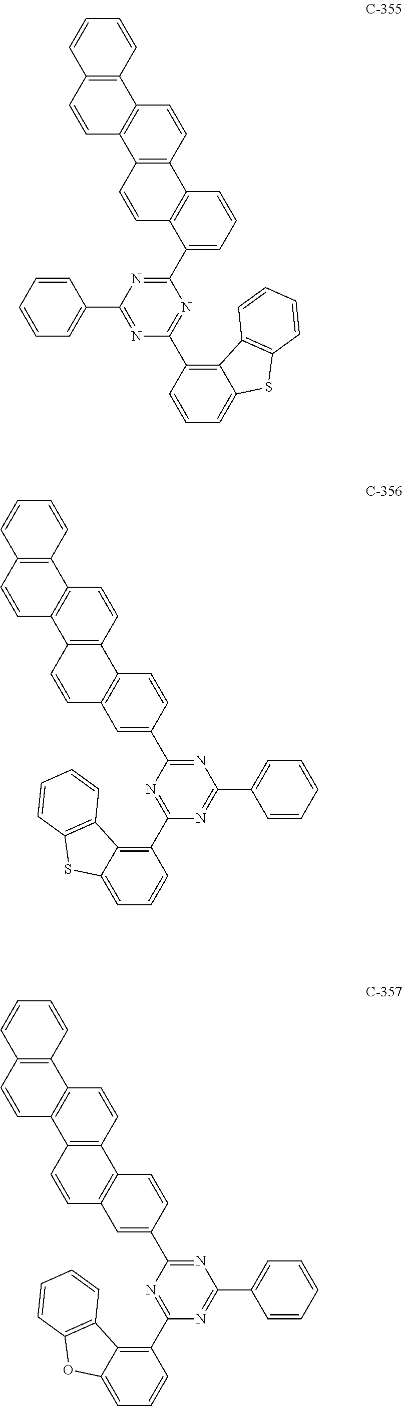 [SMILES: c1ccc(-c2nc(-c3cccc4c3ccc3c4ccc4c5ccccc5ccc43)nc(-c3cccc4sc5ccccc5c34)n2)cc1 ; c1ccc(-c2nc(-c3ccc4c(ccc5c4ccc4c6ccccc6ccc45)c3)nc(-c3cccc4sc5ccccc5c34)n2)cc1 ; c1ccc(-c2nc(-c3ccc4c(ccc5c4ccc4c6ccccc6ccc45)c3)nc(-c3cccc4oc5ccccc5c34)n2)cc1]

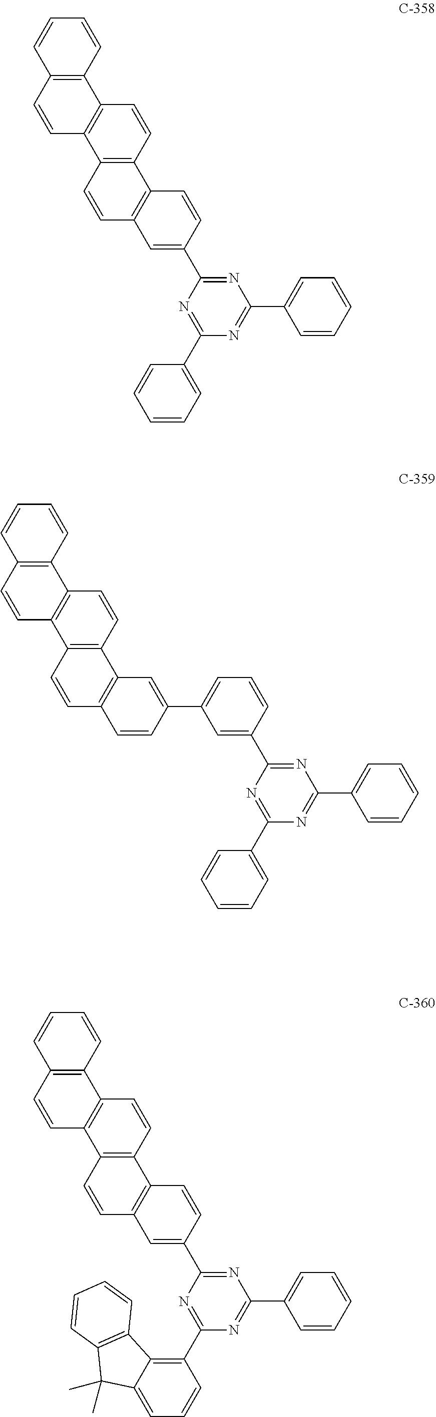 [SMILES: c1ccc(-c2nc(-c3ccccc3)nc(-c3ccc4c(ccc5c4ccc4c6ccccc6ccc45)c3)n2)cc1 ; c1ccc(-c2nc(-c3ccccc3)nc(-c3cccc(-c4ccc5ccc6c(ccc7c8ccccc8ccc76)c5c4)c3)n2)cc1 ; CC1(C)c2ccccc2-c2c(-c3nc(-c4ccccc4)nc(-c4ccc5c(ccc6c5ccc5c7ccccc7ccc56)c4)n3)cccc21]

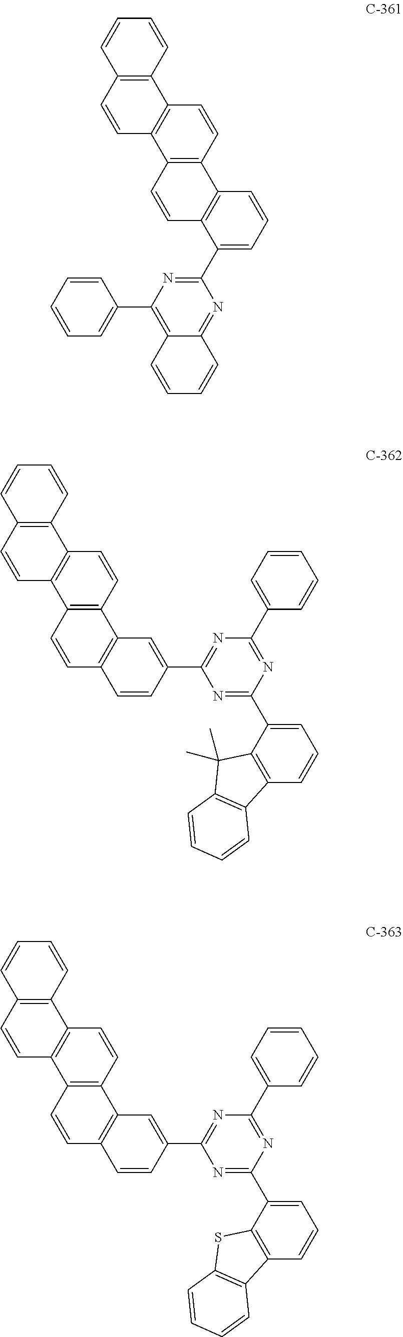 [SMILES: c1ccc(-c2nc(-c3cccc4c3ccc3c4ccc4c5ccccc5ccc43)nc3ccccc23)cc1 ; CC1(C)c2ccccc2-c2cccc(-c3nc(-c4ccccc4)nc(-c4ccc5ccc6c(ccc7c8ccccc8ccc76)c5c4)n3)c21 ; c1ccc(-c2nc(-c3ccc4ccc5c(ccc6c7ccccc7ccc65)c4c3)nc(-c3cccc4c3sc3ccccc34)n2)cc1]

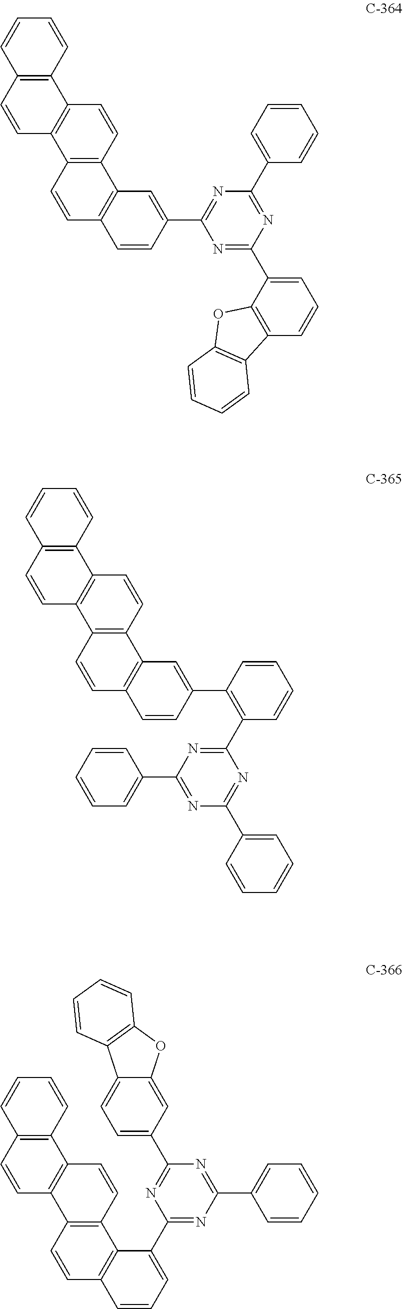 [SMILES: c1ccc(-c2nc(-c3ccc4ccc5c(ccc6c7ccccc7ccc65)c4c3)nc(-c3cccc4c3oc3ccccc34)n2)cc1 ; c1ccc(-c2nc(-c3ccccc3)nc(-c3ccccc3-c3ccc4ccc5c(ccc6c7ccccc7ccc65)c4c3)n2)cc1 ; c1ccc(-c2nc(-c3ccc4c(c3)oc3ccccc34)nc(-c3cccc4ccc5c6ccc7ccccc7c6ccc5c34)n2)cc1]

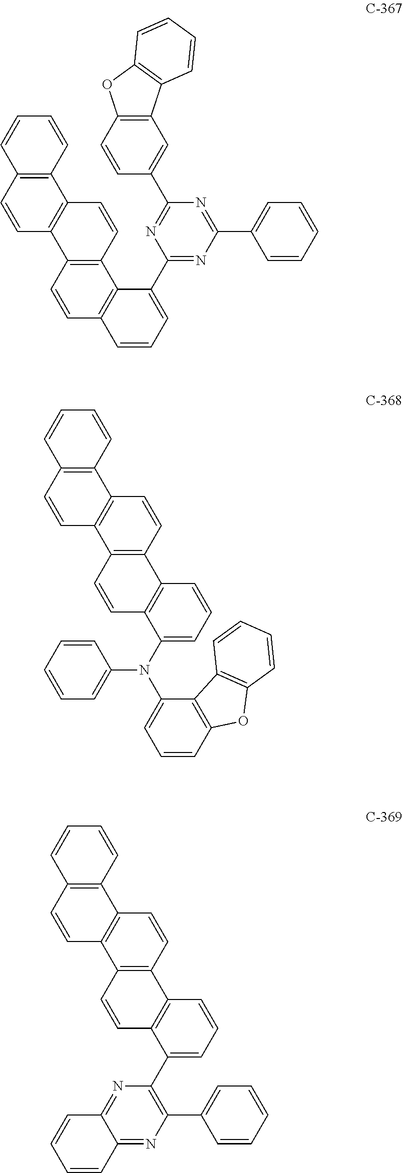 [SMILES: c1ccc(-c2nc(-c3ccc4oc5ccccc5c4c3)nc(-c3cccc4ccc5c6ccc7ccccc7c6ccc5c34)n2)cc1 ; c1ccc(N(c2cccc3c2ccc2c3ccc3c4ccccc4ccc32)c2cccc3oc4ccccc4c23)cc1 ; c1ccc(-c2nc3ccccc3nc2-c2cccc3c2ccc2c3ccc3c4ccccc4ccc32)cc1]

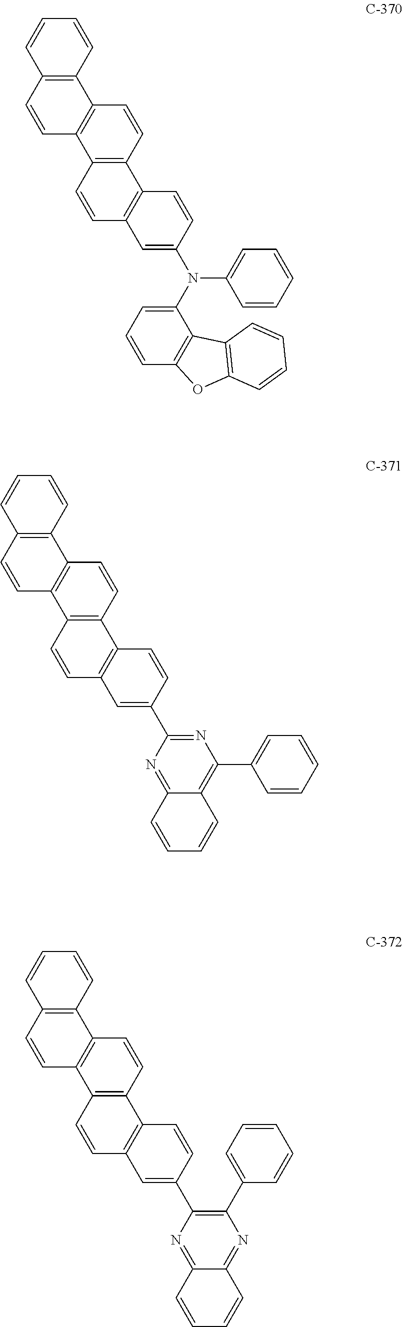 [SMILES: c1ccc(N(c2ccc3c(ccc4c3ccc3c5ccccc5ccc34)c2)c2cccc3oc4ccccc4c23)cc1 ; c1ccc(-c2nc(-c3ccc4c(ccc5c4ccc4c6ccccc6ccc45)c3)nc3ccccc23)cc1 ; c1ccc(-c2nc3ccccc3nc2-c2ccc3c(ccc4c3ccc3c5ccccc5ccc34)c2)cc1]

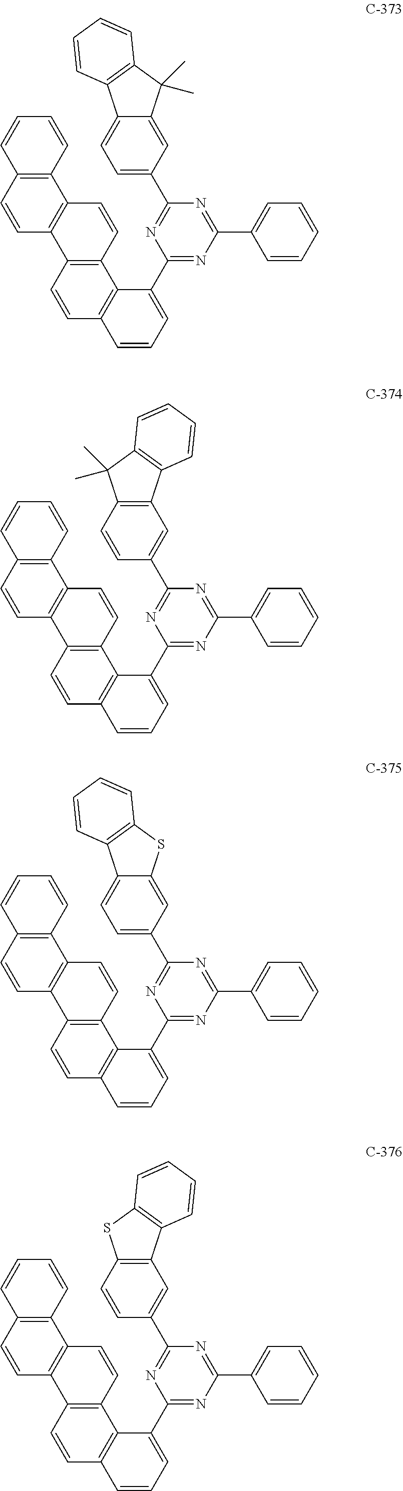 [SMILES: CC1(C)c2ccccc2-c2ccc(-c3nc(-c4ccccc4)nc(-c4cccc5ccc6c7ccc8ccccc8c7ccc6c45)n3)cc21 ; CC1(C)c2ccccc2-c2cc(-c3nc(-c4ccccc4)nc(-c4cccc5ccc6c7ccc8ccccc8c7ccc6c45)n3)ccc21 ; c1ccc(-c2nc(-c3ccc4c(c3)sc3ccccc34)nc(-c3cccc4ccc5c6ccc7ccccc7c6ccc5c34)n2)cc1 ; c1ccc(-c2nc(-c3ccc4sc5ccccc5c4c3)nc(-c3cccc4ccc5c6ccc7ccccc7c6ccc5c34)n2)cc1]

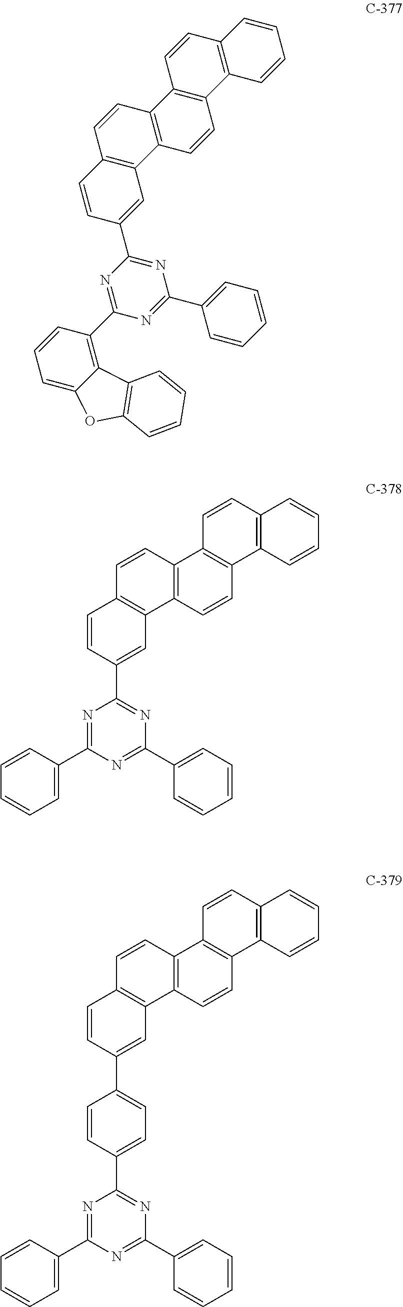 [SMILES: c1ccc(-c2nc(-c3ccc4ccc5c(ccc6c7ccccc7ccc65)c4c3)nc(-c3cccc4oc5ccccc5c34)n2)cc1 ; c1ccc(-c2nc(-c3ccccc3)nc(-c3ccc4ccc5c(ccc6c7ccccc7ccc65)c4c3)n2)cc1 ; c1ccc(-c2nc(-c3ccccc3)nc(-c3ccc(-c4ccc5ccc6c(ccc7c8ccccc8ccc76)c5c4)cc3)n2)cc1]

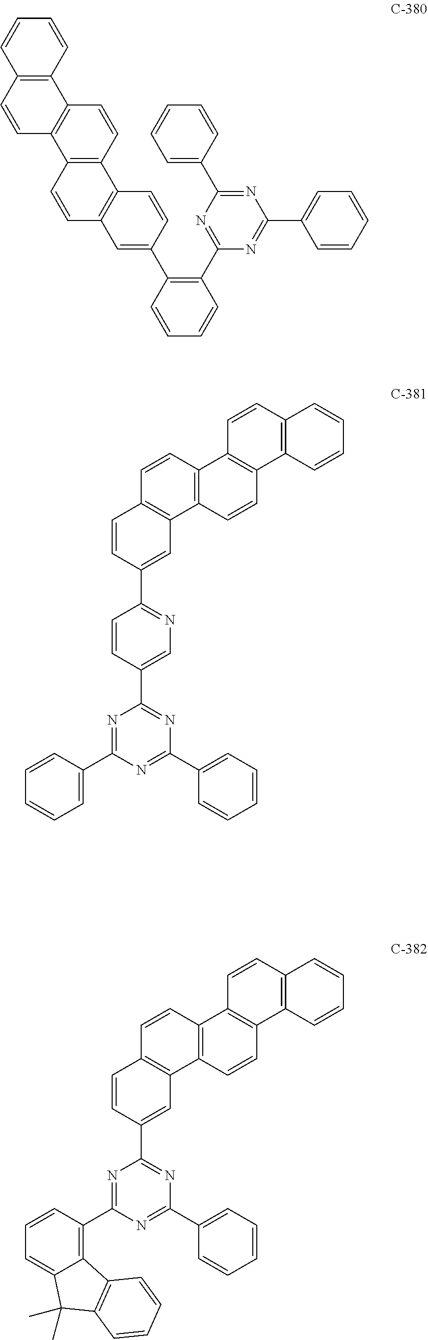 [SMILES: c1ccc(-c2nc(-c3ccccc3)nc(-c3ccccc3-c3ccc4c(ccc5c4ccc4c6ccccc6ccc45)c3)n2)cc1 ; c1ccc(-c2nc(-c3ccccc3)nc(-c3ccc(-c4ccc5ccc6c(ccc7c8ccccc8ccc76)c5c4)nc3)n2)cc1 ; CC1(C)c2ccccc2-c2c(-c3nc(-c4ccccc4)nc(-c4ccc5ccc6c(ccc7c8ccccc8ccc76)c5c4)n3)cccc21]

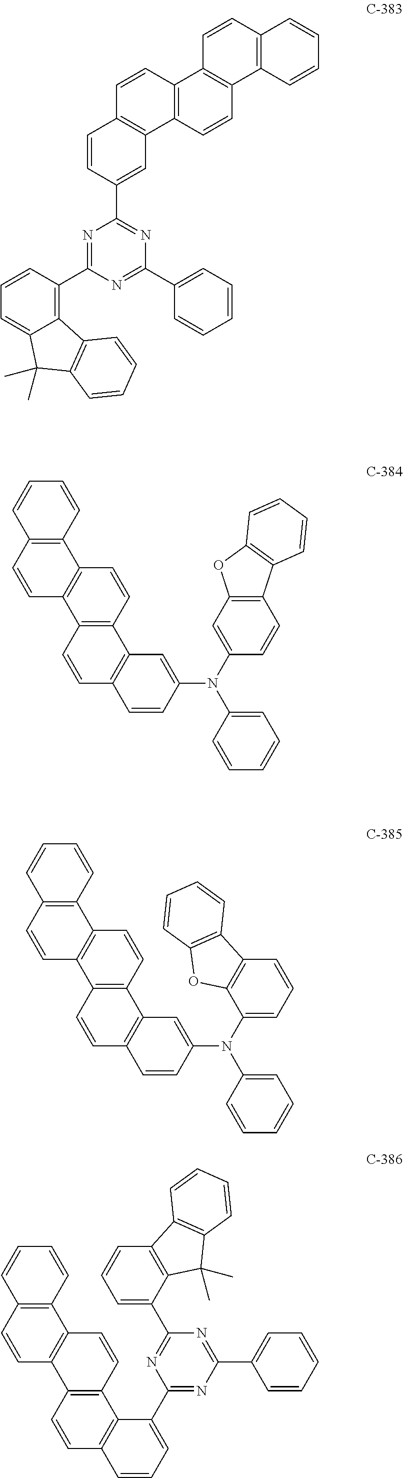 [SMILES: CC1(C)c2ccccc2-c2c(-c3nc(-c4ccccc4)nc(-c4ccc5ccc6c(ccc7c8ccccc8ccc76)c5c4)n3)cccc21 ; c1ccc(N(c2ccc3c(c2)oc2ccccc23)c2ccc3ccc4c(ccc5c6ccccc6ccc54)c3c2)cc1 ; c1ccc(N(c2ccc3ccc4c(ccc5c6ccccc6ccc54)c3c2)c2cccc3c2oc2ccccc23)cc1 ; CC1(C)c2ccccc2-c2cccc(-c3nc(-c4ccccc4)nc(-c4cccc5ccc6c7ccc8ccccc8c7ccc6c45)n3)c21]

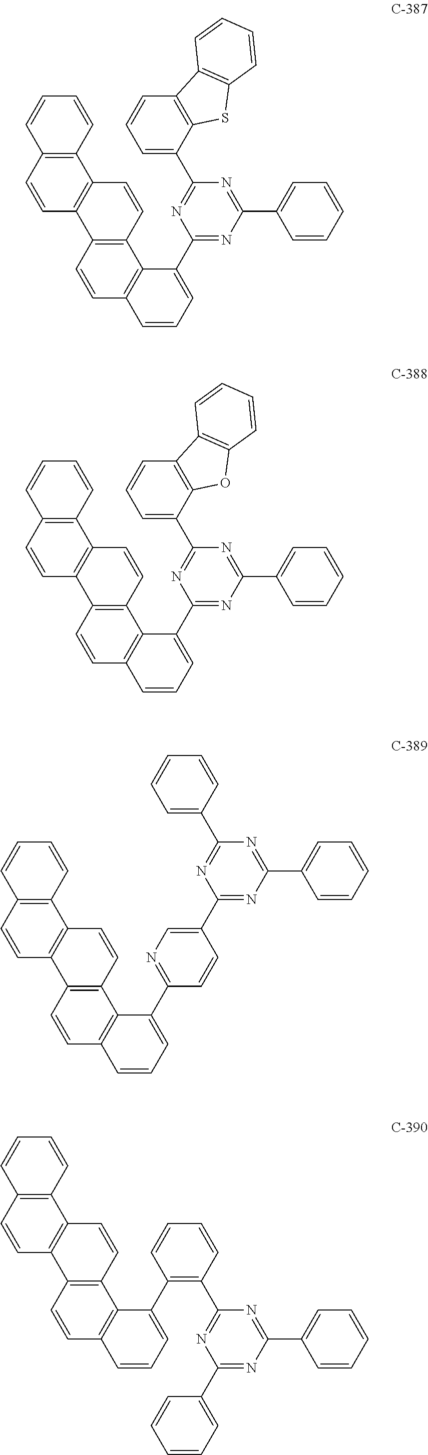 [SMILES: c1ccc(-c2nc(-c3cccc4c3sc3ccccc34)nc(-c3cccc4ccc5c6ccc7ccccc7c6ccc5c34)n2)cc1 ; c1ccc(-c2nc(-c3cccc4c3oc3ccccc34)nc(-c3cccc4ccc5c6ccc7ccccc7c6ccc5c34)n2)cc1 ; c1ccc(-c2nc(-c3ccccc3)nc(-c3ccc(-c4cccc5ccc6c7ccc8ccccc8c7ccc6c45)nc3)n2)cc1 ; c1ccc(-c2nc(-c3ccccc3)nc(-c3ccccc3-c3cccc4ccc5c6ccc7ccccc7c6ccc5c34)n2)cc1]

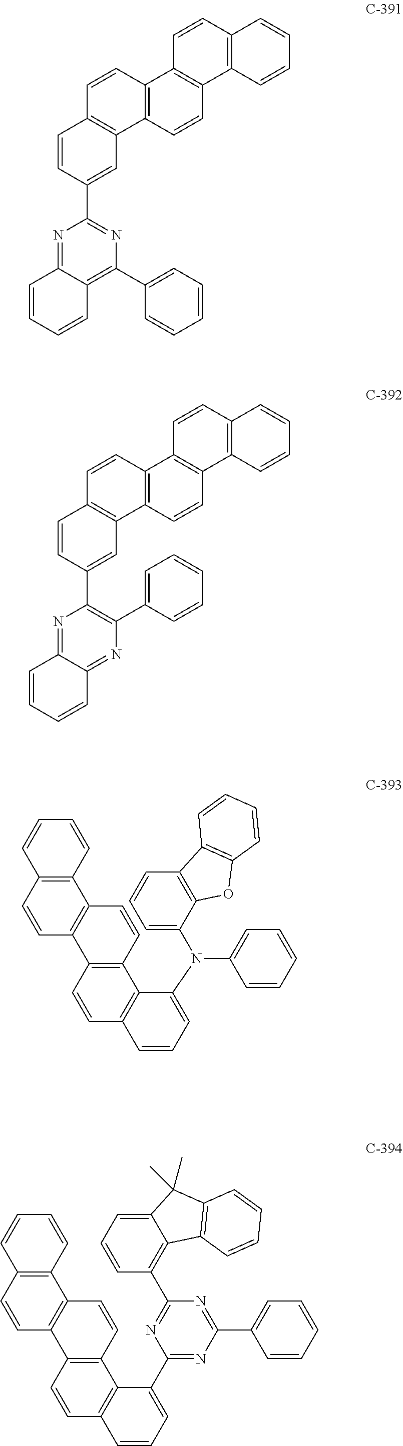 [SMILES: c1ccc(-c2nc(-c3ccc4ccc5c(ccc6c7ccccc7ccc65)c4c3)nc3ccccc23)cc1 ; c1ccc(-c2nc3ccccc3nc2-c2ccc3ccc4c(ccc5c6ccccc6ccc54)c3c2)cc1 ; c1ccc(N(c2cccc3c2oc2ccccc23)c2cccc3ccc4c5ccc6ccccc6c5ccc4c23)cc1 ; CC1(C)c2ccccc2-c2c(-c3nc(-c4ccccc4)nc(-c4cccc5ccc6c7ccc8ccccc8c7ccc6c45)n3)cccc21]

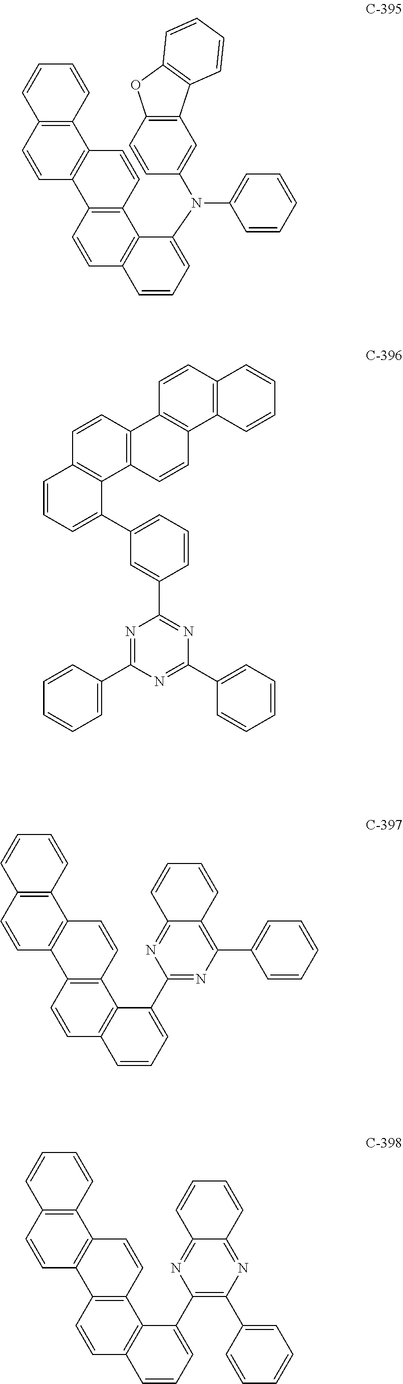 [SMILES: c1ccc(N(c2ccc3oc4ccccc4c3c2)c2cccc3ccc4c5ccc6ccccc6c5ccc4c23)cc1 ; c1ccc(-c2nc(-c3ccccc3)nc(-c3cccc(-c4cccc5ccc6c7ccc8ccccc8c7ccc6c45)c3)n2)cc1 ; c1ccc(-c2nc(-c3cccc4ccc5c6ccc7ccccc7c6ccc5c34)nc3ccccc23)cc1 ; c1ccc(-c2nc3ccccc3nc2-c2cccc3ccc4c5ccc6ccccc6c5ccc4c23)cc1]

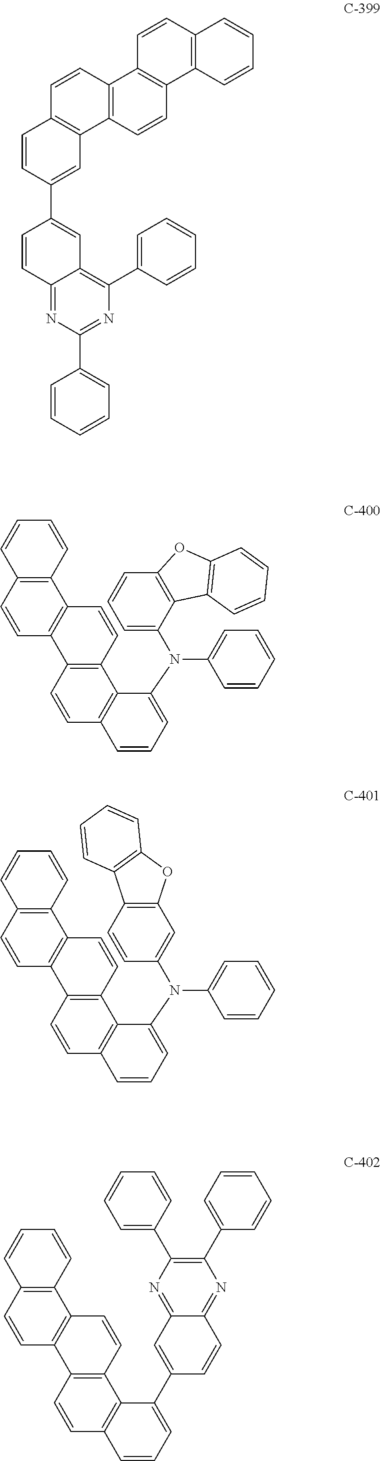 [SMILES: c1ccc(-c2nc(-c3ccccc3)c3cc(-c4ccc5ccc6c(ccc7c8ccccc8ccc76)c5c4)ccc3n2)cc1 ; c1ccc(N(c2cccc3ccc4c5ccc6ccccc6c5ccc4c23)c2cccc3oc4ccccc4c23)cc1 ; c1ccc(N(c2ccc3c(c2)oc2ccccc23)c2cccc3ccc4c5ccc6ccccc6c5ccc4c23)cc1 ; c1ccc(-c2nc3ccc(-c4cccc5ccc6c7ccc8ccccc8c7ccc6c45)cc3nc2-c2ccccc2)cc1]

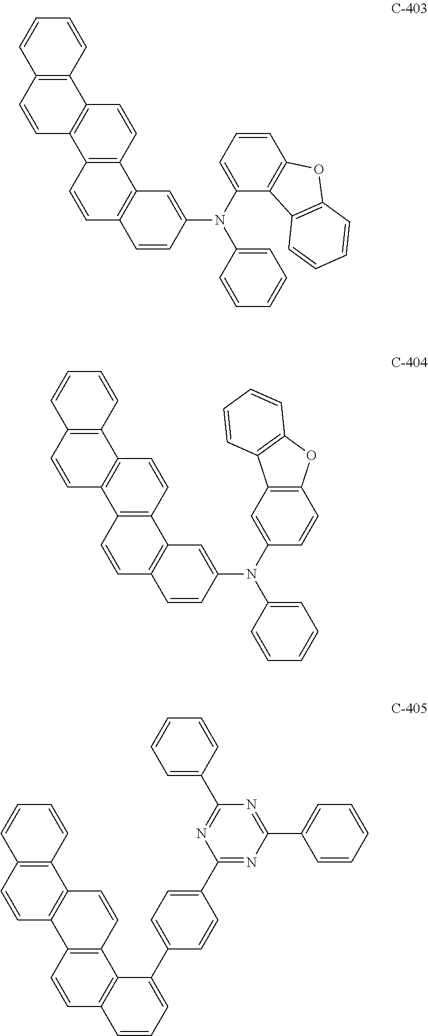 [SMILES: c1ccc(N(c2ccc3ccc4c(ccc5c6ccccc6ccc54)c3c2)c2cccc3oc4ccccc4c23)cc1 ; c1ccc(N(c2ccc3ccc4c(ccc5c6ccccc6ccc54)c3c2)c2ccc3oc4ccccc4c3c2)cc1 ; c1ccc(-c2nc(-c3ccccc3)nc(-c3ccc(-c4cccc5ccc6c7ccc8ccccc8c7ccc6c45)cc3)n2)cc1]

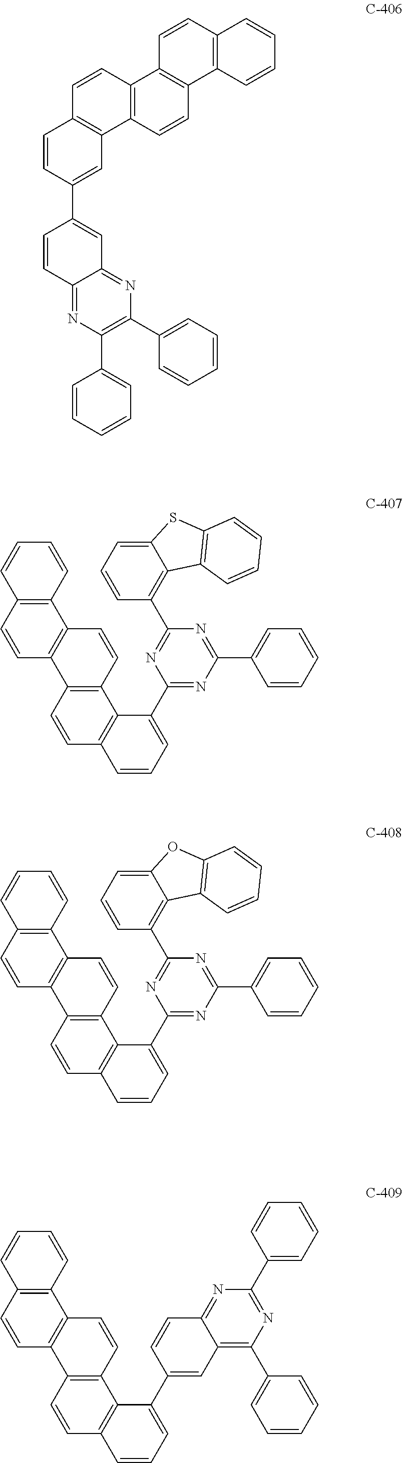 [SMILES: c1ccc(-c2nc3ccc(-c4ccc5ccc6c(ccc7c8ccccc8ccc76)c5c4)cc3nc2-c2ccccc2)cc1 ; c1ccc(-c2nc(-c3cccc4ccc5c6ccc7ccccc7c6ccc5c34)nc(-c3cccc4sc5ccccc5c34)n2)cc1 ; c1ccc(-c2nc(-c3cccc4ccc5c6ccc7ccccc7c6ccc5c34)nc(-c3cccc4oc5ccccc5c34)n2)cc1 ; c1ccc(-c2nc(-c3ccccc3)c3cc(-c4cccc5ccc6c7ccc8ccccc8c7ccc6c45)ccc3n2)cc1]

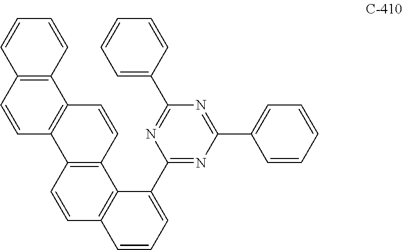 [SMILES: c1ccc(-c2nc(-c3ccccc3)nc(-c3cccc4ccc5c6ccc7ccccc7c6ccc5c34)n2)cc1]

The compound of formula 1 according to the present disclosure may be prepared by a known synthetic method, and in particular, a synthetic method disclosed in a number of patent documents may be used.

The present disclosure provides an organic electroluminescent material comprising the organic electroluminescent compound of formula 1, and an organic electroluminescent device comprising the organic electroluminescent material.

The organic electroluminescent material may be made of the organic electroluminescent compound of the present disclosure alone, or may further include conventional materials included in the organic electroluminescent material. When two or more materials are included in one layer, they may be mixture-evaporated to form a layer, or may be separately and simultaneously co-evaporated to form a layer. The organic electroluminescent material according to one embodiment may include at least one compound represented by the formula 1. For example, the compound of the formula 1 may be included in the light-emitting layer, and when included in the light-emitting layer, the compound of the formula 1 may be included as a host, and more specifically, may be included as a phosphorescent host.

According to one embodiment, the present disclosure provides a plurality of host materials comprising a first host material represented by the formula 1 and a second host material which is different from the first host material.

The second host material according to one embodiment comprises an organic electroluminescent compound represented by the following formula 2.

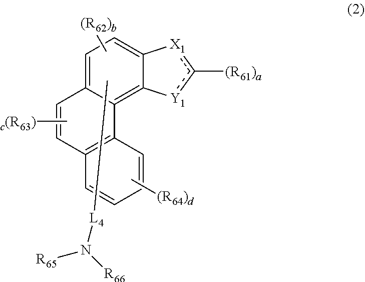

In formula 2,

X₁ and Y₁ each independently represent —N═, —NR₆₇—, —O— or —S—, provided that one of X₁ and Y₁ is —N═, and the other of X₁ and Y₁ is —NR₆₇—, —O— or —S—;

R₆₁ represents a substituted or unsubstituted (C6-C30)aryl or a substituted or unsubstituted (3- to 30-membered)heteroaryl;

R₆₂ to R₆₄ and R₆₇ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of a (C3-C30) aliphatic ring and a (C6-C30) aromatic ring, or -L₃″-N(Ar₃″)(Ar₄″); or may be linked to the adjacent substituent each other to form a ring(s);

L₃″ represents a single bond, a substituted or unsubstituted (C6-C30)arylene, or a substituted or unsubstituted (3- to 30-membered)heteroarylene;

Ar₃″ and Ar₄″ each independently represent hydrogen, a substituted or unsubstituted (C1-C30)alkyl, a substituted or unsubstituted (C2-C30)alkenyl, a substituted or unsubstituted fused ring of a (C3-C30) aliphatic ring and a (C6-C30) aromatic ring, a substituted or unsubstituted (C6-C30)aryl, or a substituted or unsubstituted (3- to 30-membered)heteroaryl;

L₄ represents a single bond, a substituted or unsubstituted (C6-C30)arylene, or a substituted or unsubstituted (3- to 30-membered)heteroarylene;

R₆₅ and R₆₆ each independently represent a substituted or unsubstituted (C6-C30)aryl or a substituted or unsubstituted (3- to 30-membered)heteroaryl; and

a is 1, b and c each independently represent 1 or 2, and d is an integer of 1 to 4, and when b to d are an integer of 2 or more, each of R₆₂ to R₆₄ may be the same or different.

In one embodiment, one of X₁ and Y₁ may be —N═, and the other of X₁ and Y₁ may be —O— or —S—. For example, X₁ may be —N═ and Y₁ may be —O—, or X₁ may be —O— and Y₁ may be —N═, or X₁ may be —S— and Y₁ may be —N═.

In one embodiment, R₆₁ may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C18)aryl or a substituted or unsubstituted (5- to 18-membered)heteroaryl. For example, R₆₁ may be an unsubstituted phenyl, an unsubstituted naphthyl, an unsubstituted o-biphenyl, an unsubstituted m-biphenyl, an unsubstituted p-biphenyl, or an unsubstituted pyridyl.

In one embodiment, R₆₂ to R₆₄ and R₆₇ each independently may be hydrogen, deuterium, halogen, cyano, a substituted or unsubstituted (C1-C30)alkyl, or a substituted or unsubstituted (C6-C30)aryl, preferably hydrogen, deuterium, halogen, cyano, or a substituted or unsubstituted (C6-C25)aryl, more preferably hydrogen, deuterium, or a substituted or unsubstituted (C6-C18)aryl. For example, R₆₂ to R₆₄ each independently may be hydrogen or an unsubstituted phenyl.

In one embodiment, L₄ may be a single bond or a substituted or unsubstituted (C6-C30)arylene, preferably a single bond or a substituted or unsubstituted (C6-C25)arylene, more preferably a substituted or unsubstituted (CM-C18)arylene. For example, L₄ may be a single bond, an unsubstituted phenylene, or an unsubstituted naphthylene.

In one embodiment, R₆₅ and R₆₆ each independently may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C25)aryl or a substituted or unsubstituted (5- to 20-membered)heteroaryl. For example, R₆₅ and R₆₆ each independently may be a substituted or unsubstituted phenyl, a substituted or unsubstituted o-biphenyl, a substituted or unsubstituted m-biphenyl, a substituted or unsubstituted p-biphenyl, a substituted or unsubstituted naphthyl, a substituted or unsubstituted terphenyl, a substituted or unsubstituted fluorenyl, a substituted or unsubstituted phenanthrenyl, a substituted or unsubstituted benzofuranyl, a substituted or unsubstituted dibenzofuranyl, a substituted or unsubstituted dibenzothiophenyl, a substituted or unsubstituted benzofluorenyl, a substituted or unsubstituted spirobifluorenyl, a substituted or unsubstituted C22 aryl, a substituted or unsubstituted carbazolyl, a substituted or unsubstituted benzonaphthofuranyl, or a substituted or unsubstituted benzofuropyridyl. The substituents of the substituted groups may be at least one of deuterium; methyl; tert-butyl; cyclohexyl; phenyl unsubstituted or substituted with at least one of methyl and tert-butyl; naphthyl; biphenyl; anthracenyl; fluoranthenyl; phenylfluorenyl; pyridyl unsubstituted or substituted with phenyl; phenoxazinyl; diphenylamino; and benzimidazolyl substituted with phenyl.

According to one embodiment, the organic electroluminescent compound of formula 2 may be more specifically illustrated by the following compounds, but is not limited thereto.

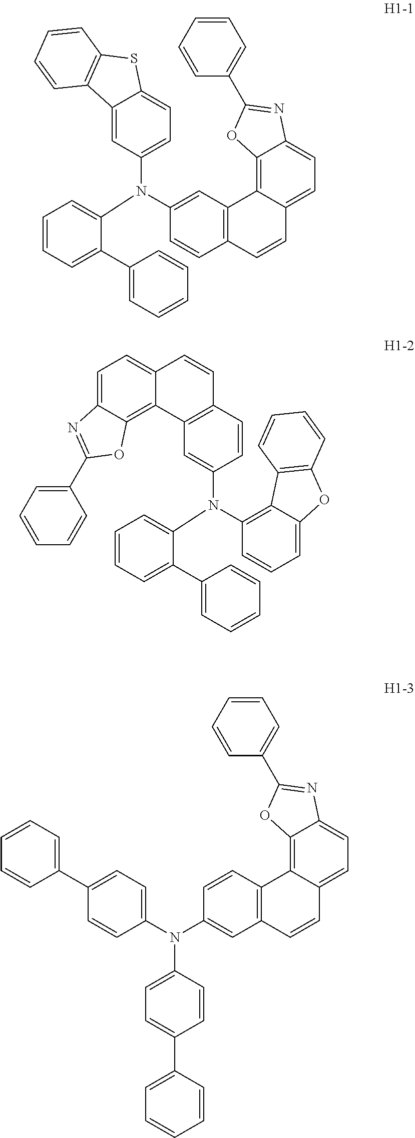

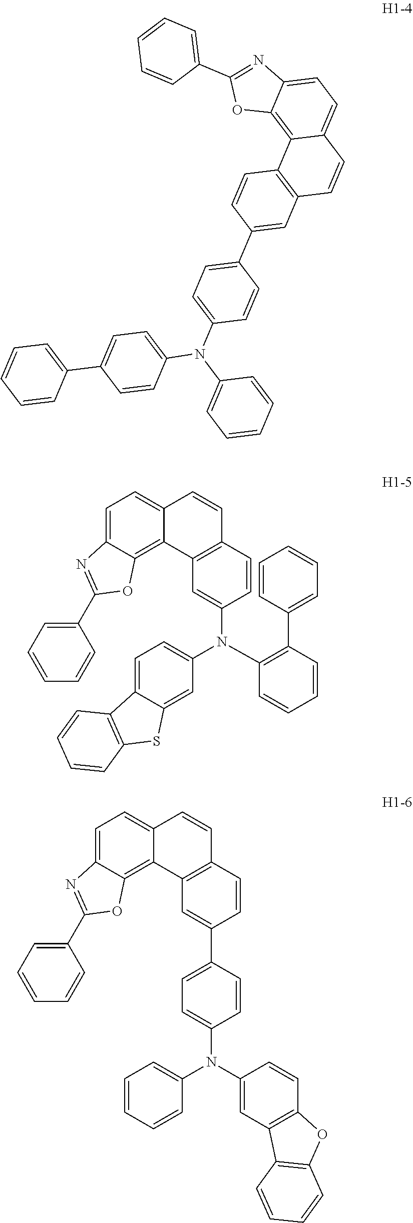

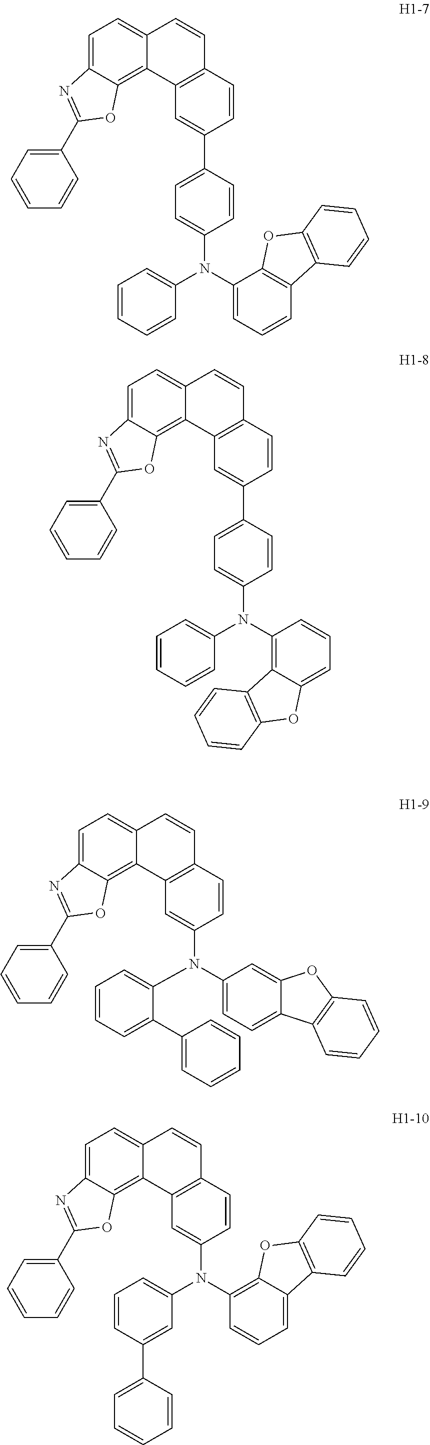

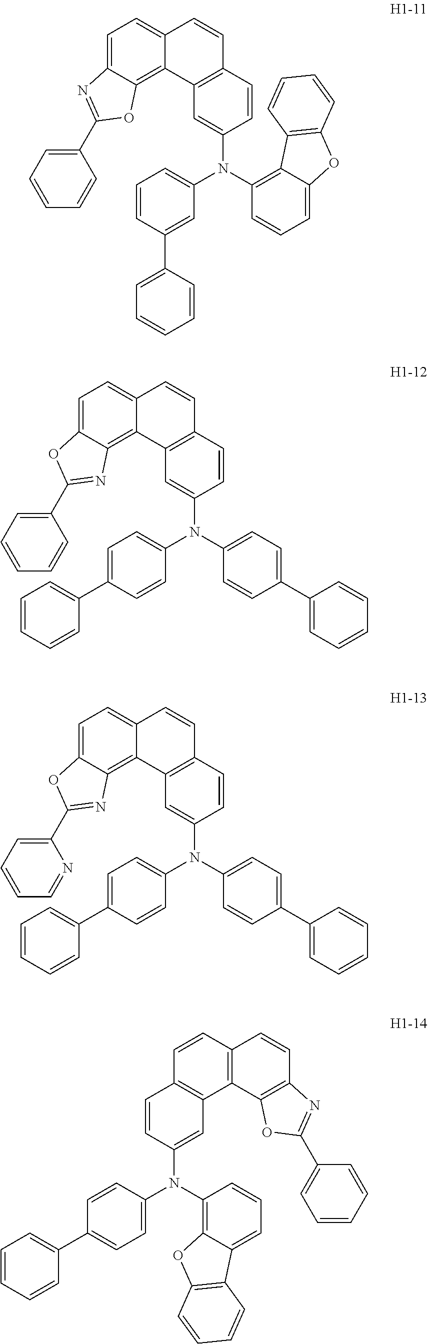 [SMILES: c1ccc(-c2cccc(N(c3ccc4ccc5ccc6nc(-c7ccccc7)oc6c5c4c3)c3cccc4oc5ccccc5c34)c2)cc1 ; c1ccc(-c2ccc(N(c3ccc(-c4ccccc4)cc3)c3ccc4ccc5ccc6oc(-c7ccccc7)nc6c5c4c3)cc2)cc1 ; c1ccc(-c2ccc(N(c3ccc(-c4ccccc4)cc3)c3ccc4ccc5ccc6oc(-c7ccccn7)nc6c5c4c3)cc2)cc1 ; c1ccc(-c2ccc(N(c3ccc4ccc5ccc6nc(-c7ccccc7)oc6c5c4c3)c3cccc4c3oc3ccccc34)cc2)cc1]

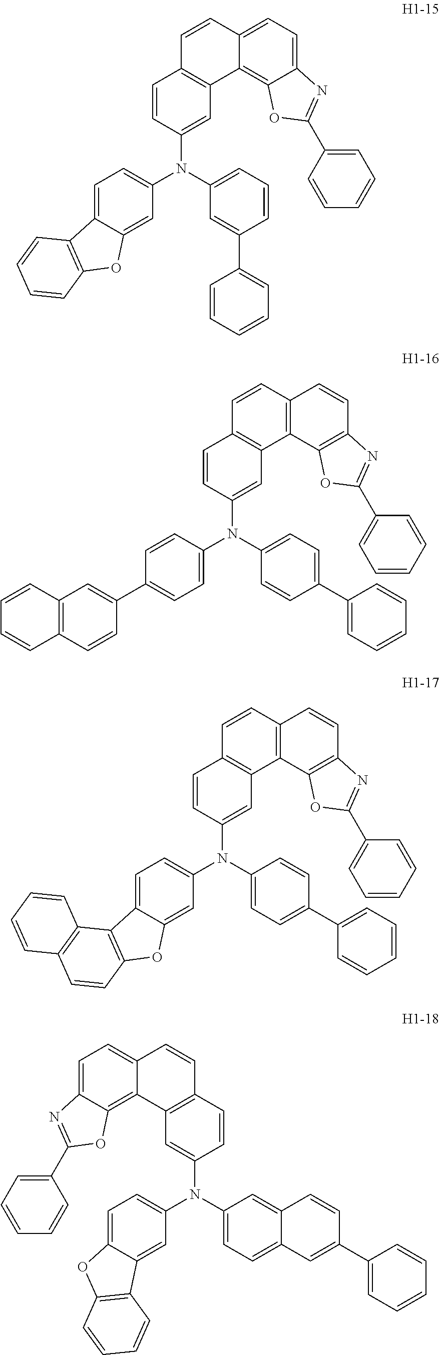 [SMILES: c1ccc(-c2cccc(N(c3ccc4c(c3)oc3ccccc34)c3ccc4ccc5ccc6nc(-c7ccccc7)oc6c5c4c3)c2)cc1 ; c1ccc(-c2ccc(N(c3ccc(-c4ccc5ccccc5c4)cc3)c3ccc4ccc5ccc6nc(-c7ccccc7)oc6c5c4c3)cc2)cc1 ; c1ccc(-c2ccc(N(c3ccc4c(c3)oc3ccc5ccccc5c34)c3ccc4ccc5ccc6nc(-c7ccccc7)oc6c5c4c3)cc2)cc1 ; c1ccc(-c2ccc3cc(N(c4ccc5oc6ccccc6c5c4)c4ccc5ccc6ccc7nc(-c8ccccc8)oc7c6c5c4)ccc3c2)cc1]

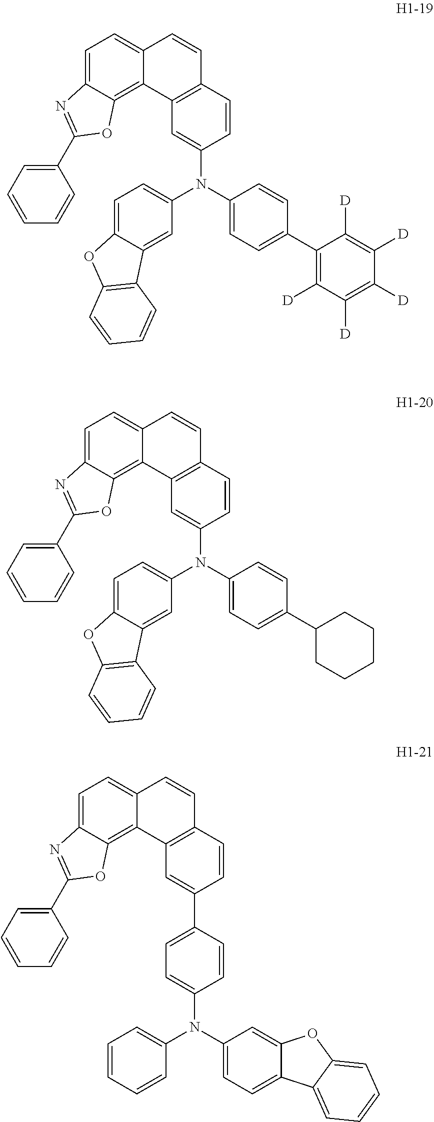 [SMILES: [2H]c1c([2H])c([2H])c(-c2ccc(N(c3ccc4oc5ccccc5c4c3)c3ccc4ccc5ccc6nc(-c7ccccc7)oc6c5c4c3)cc2)c([2H])c1[2H] ; c1ccc(-c2nc3ccc4ccc5ccc(N(c6ccc(C7CCCCC7)cc6)c6ccc7oc8ccccc8c7c6)cc5c4c3o2)cc1 ; c1ccc(-c2nc3ccc4ccc5ccc(-c6ccc(N(c7ccccc7)c7ccc8c(c7)oc7ccccc78)cc6)cc5c4c3o2)cc1]

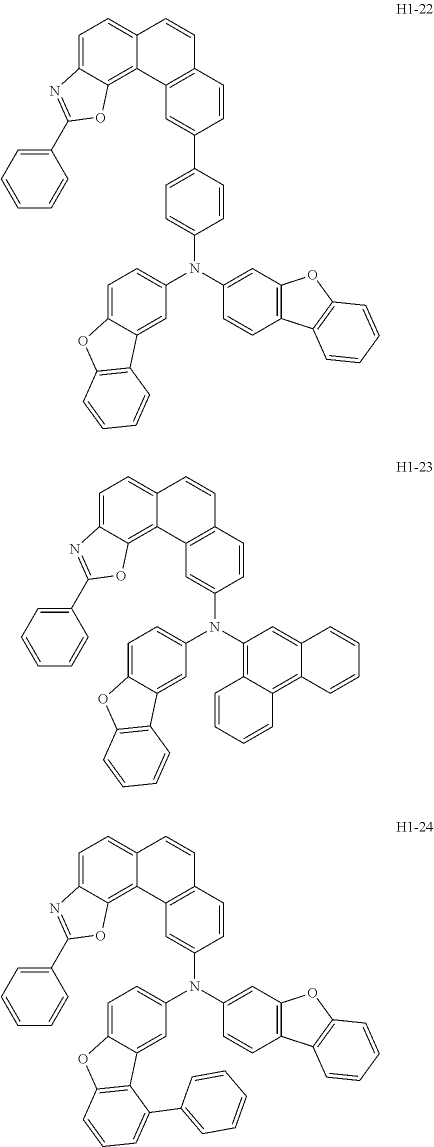 [SMILES: c1ccc(-c2nc3ccc4ccc5ccc(-c6ccc(N(c7ccc8c(c7)oc7ccccc78)c7ccc8oc9ccccc9c8c7)cc6)cc5c4c3o2)cc1 ; c1ccc(-c2nc3ccc4ccc5ccc(N(c6ccc7oc8ccccc8c7c6)c6cc7ccccc7c7ccccc67)cc5c4c3o2)cc1 ; c1ccc(-c2nc3ccc4ccc5ccc(N(c6ccc7c(c6)oc6ccccc67)c6ccc7oc8cccc(-c9ccccc9)c8c7c6)cc5c4c3o2)cc1]

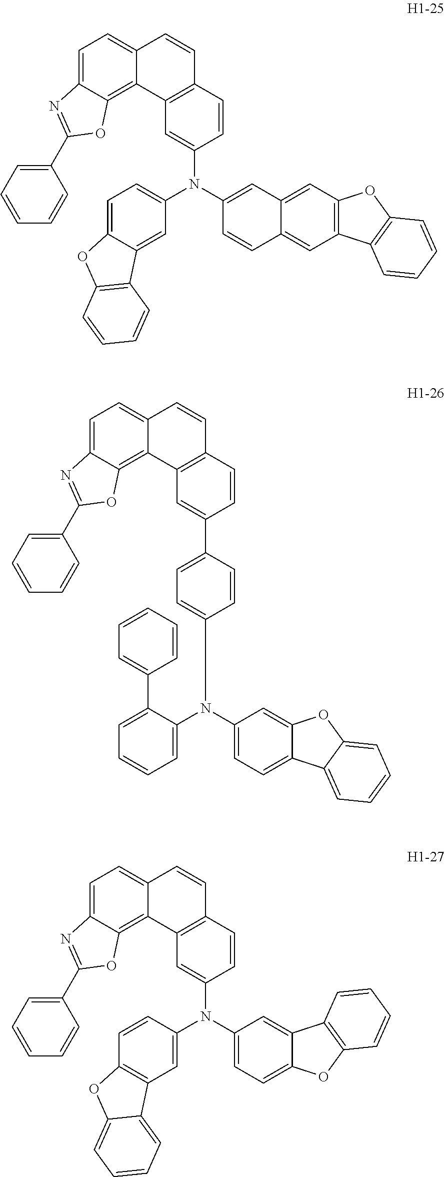 [SMILES: c1ccc(-c2nc3ccc4ccc5ccc(N(c6ccc7cc8c(cc7c6)oc6ccccc68)c6ccc7oc8ccccc8c7c6)cc5c4c3o2)cc1 ; c1ccc(-c2nc3ccc4ccc5ccc(-c6ccc(N(c7ccc8c(c7)oc7ccccc78)c7ccccc7-c7ccccc7)cc6)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1]

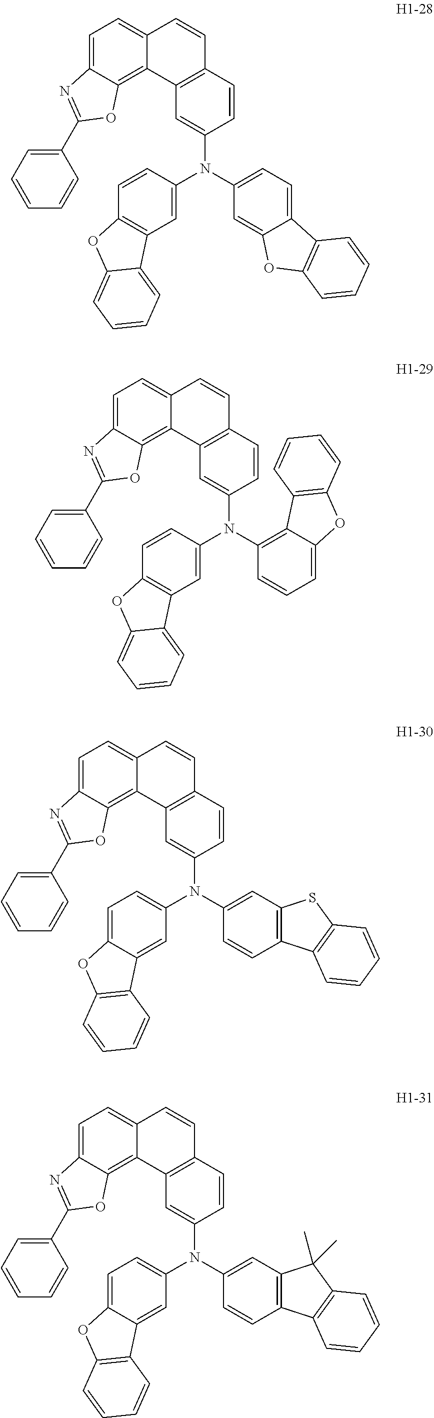 [SMILES: c1ccc(-c2nc3ccc4ccc5ccc(N(c6ccc7c(c6)oc6ccccc67)c6ccc7oc8ccccc8c7c6)cc5c4c3o2)cc1 ; c1ccc(-c2nc3ccc4ccc5ccc(N(c6ccc7oc8ccccc8c7c6)c6cccc7oc8ccccc8c67)cc5c4c3o2)cc1 ; c1ccc(-c2nc3ccc4ccc5ccc(N(c6ccc7c(c6)sc6ccccc67)c6ccc7oc8ccccc8c7c6)cc5c4c3o2)cc1 ; CC1(C)c2ccccc2-c2ccc(N(c3ccc4oc5ccccc5c4c3)c3ccc4ccc5ccc6nc(-c7ccccc7)oc6c5c4c3)cc21]

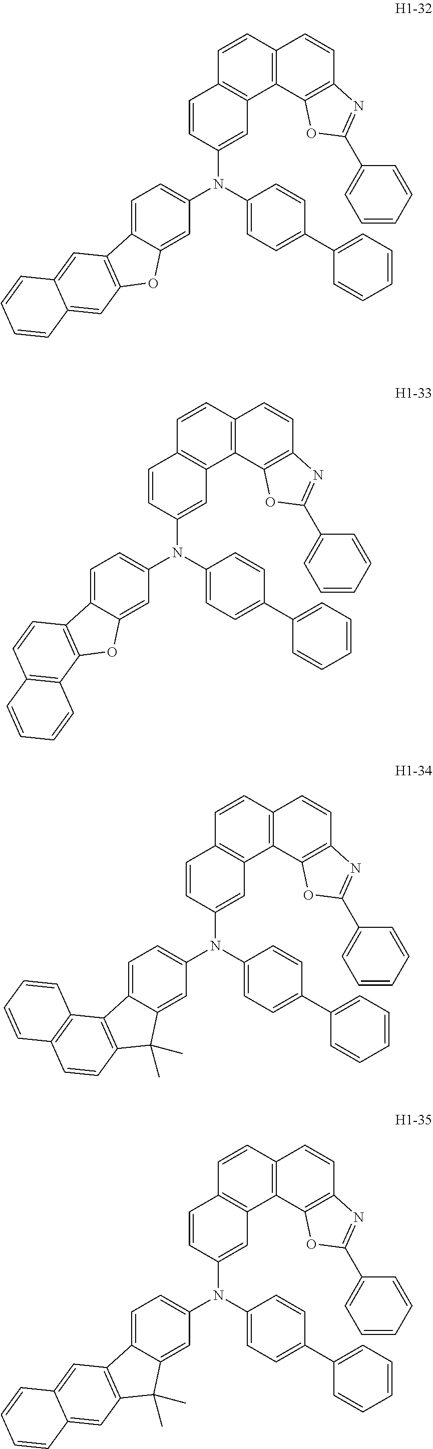 [SMILES: c1ccc(-c2ccc(N(c3ccc4c(c3)oc3cc5ccccc5cc34)c3ccc4ccc5ccc6nc(-c7ccccc7)oc6c5c4c3)cc2)cc1 ; c1ccc(-c2ccc(N(c3ccc4c(c3)oc3c5ccccc5ccc43)c3ccc4ccc5ccc6nc(-c7ccccc7)oc6c5c4c3)cc2)cc1 ; CC1(C)c2cc(N(c3ccc(-c4ccccc4)cc3)c3ccc4ccc5ccc6nc(-c7ccccc7)oc6c5c4c3)ccc2-c2c1ccc1ccccc21 ; CC1(C)c2cc(N(c3ccc(-c4ccccc4)cc3)c3ccc4ccc5ccc6nc(-c7ccccc7)oc6c5c4c3)ccc2-c2cc3ccccc3cc21]

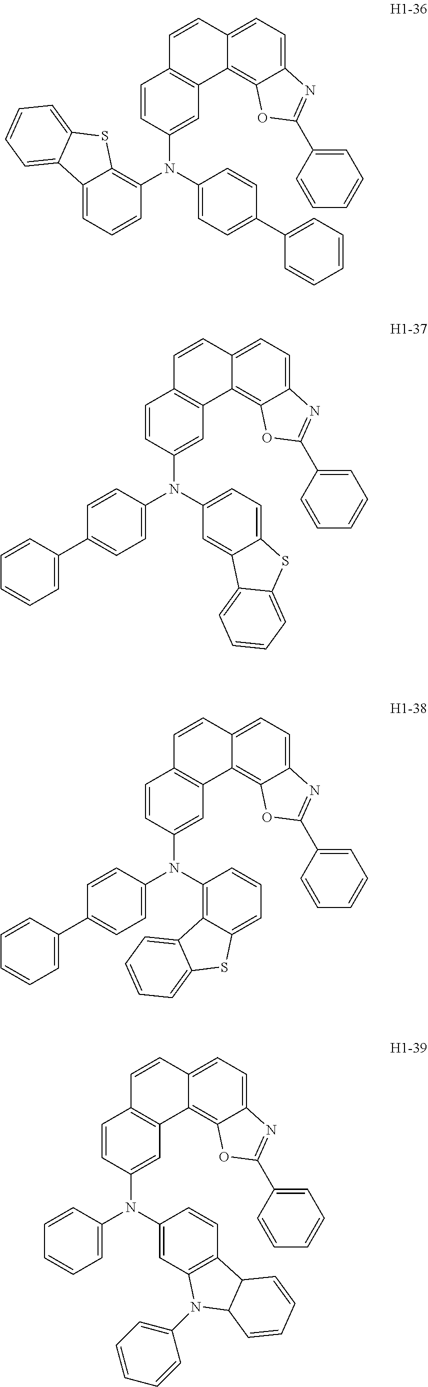 [SMILES: c1ccc(-c2ccc(N(c3ccc4ccc5ccc6nc(-c7ccccc7)oc6c5c4c3)c3cccc4c3sc3ccccc34)cc2)cc1 ; c1ccc(-c2ccc(N(c3ccc4sc5ccccc5c4c3)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4sc5ccccc5c34)cc2)cc1 ; C1=CC2c3ccc(N(c4ccccc4)c4ccc5ccc6ccc7nc(-c8ccccc8)oc7c6c5c4)cc3N(c3ccccc3)C2C=C1]

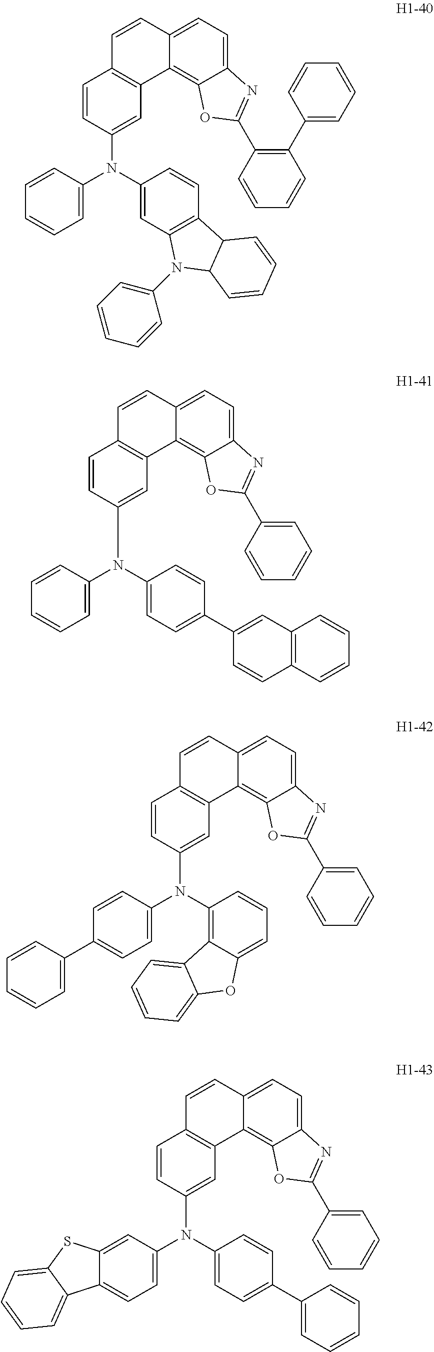

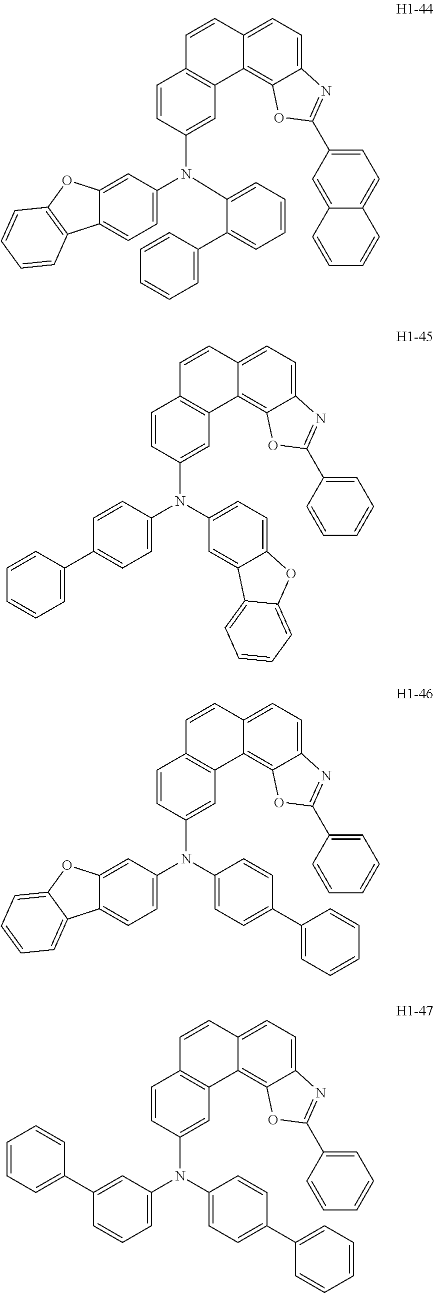

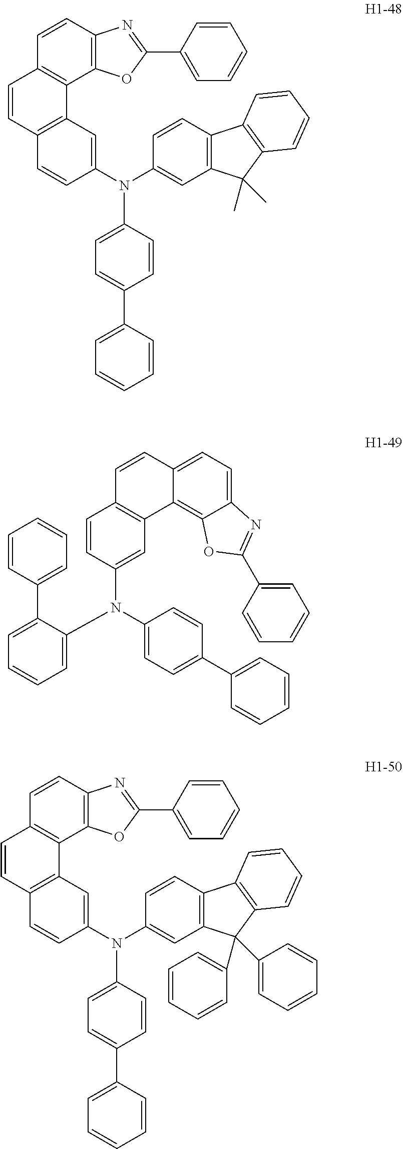 [SMILES: CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3ccc4ccc5ccc6nc(-c7ccccc7)oc6c5c4c3)c3ccccc3-c3ccccc3)cc2)cc1 ; c1ccc(-c2ccc(N(c3ccc4c(c3)C(c3ccccc3)(c3ccccc3)c3ccccc3-4)c3ccc4ccc5ccc6nc(-c7ccccc7)oc6c5c4c3)cc2)cc1]

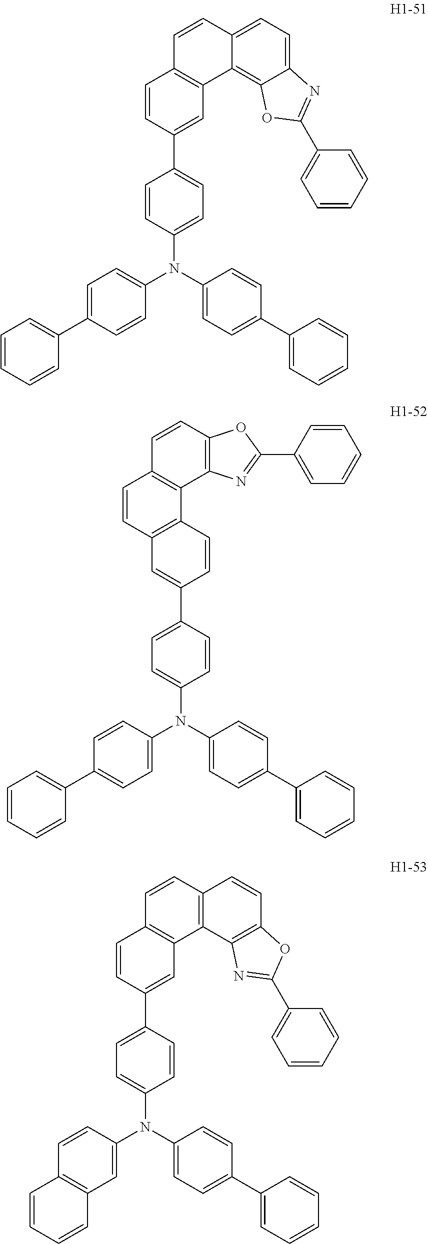

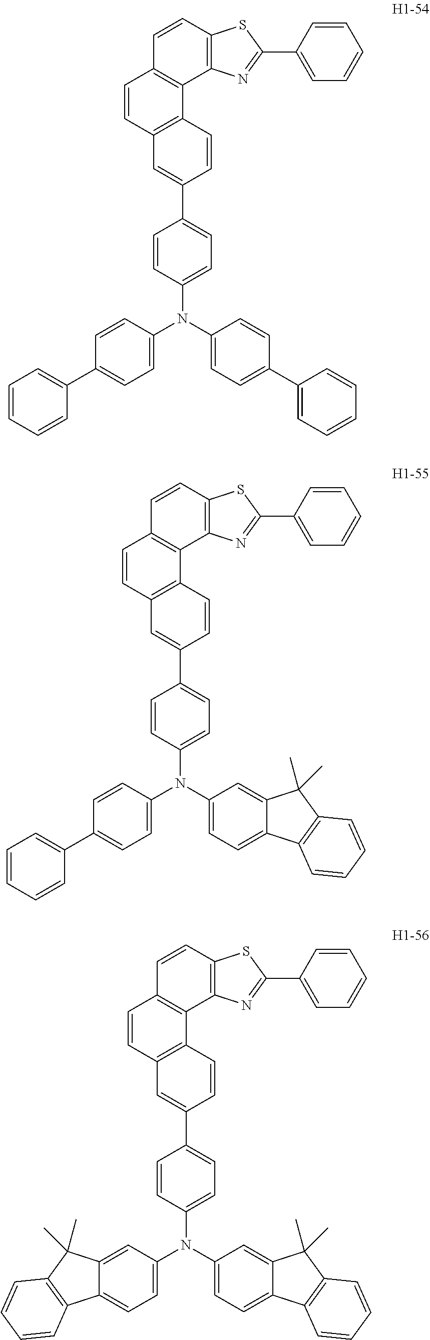 [SMILES: c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21]

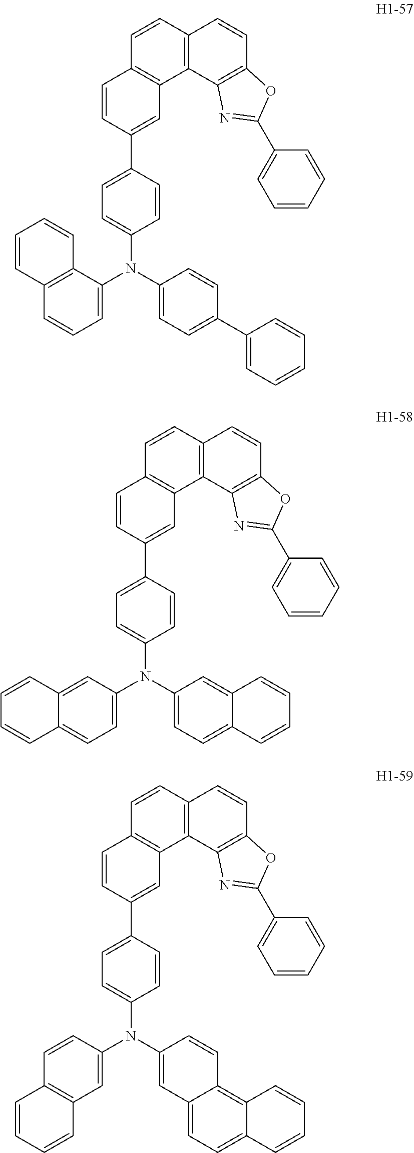

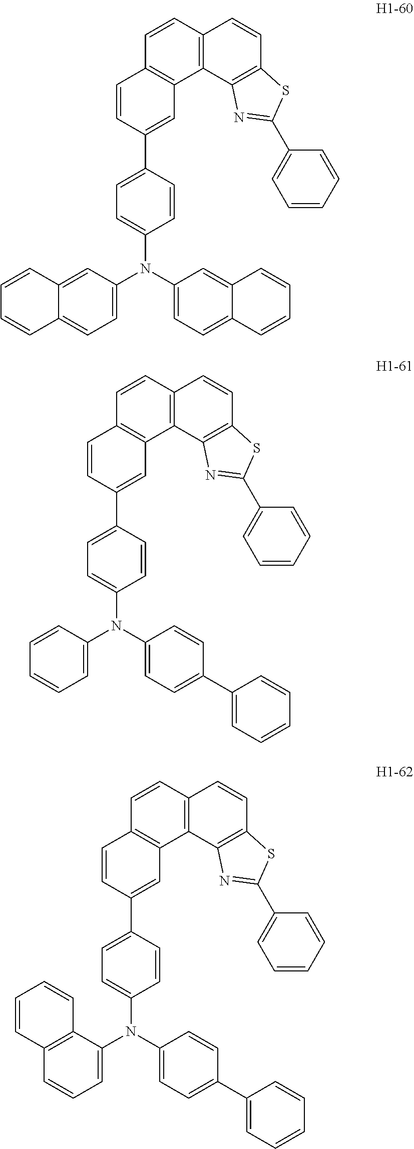

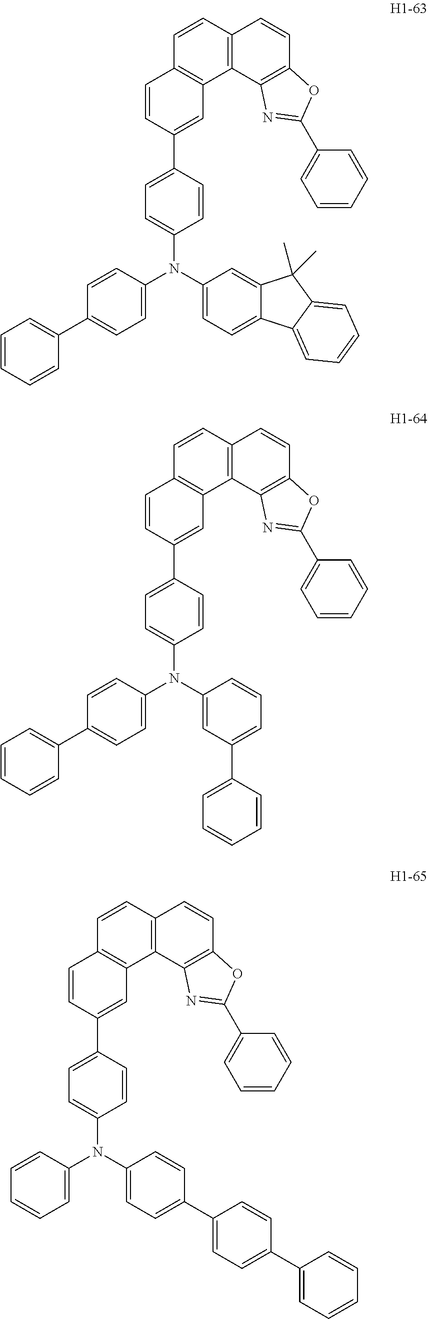 [SMILES: CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1]

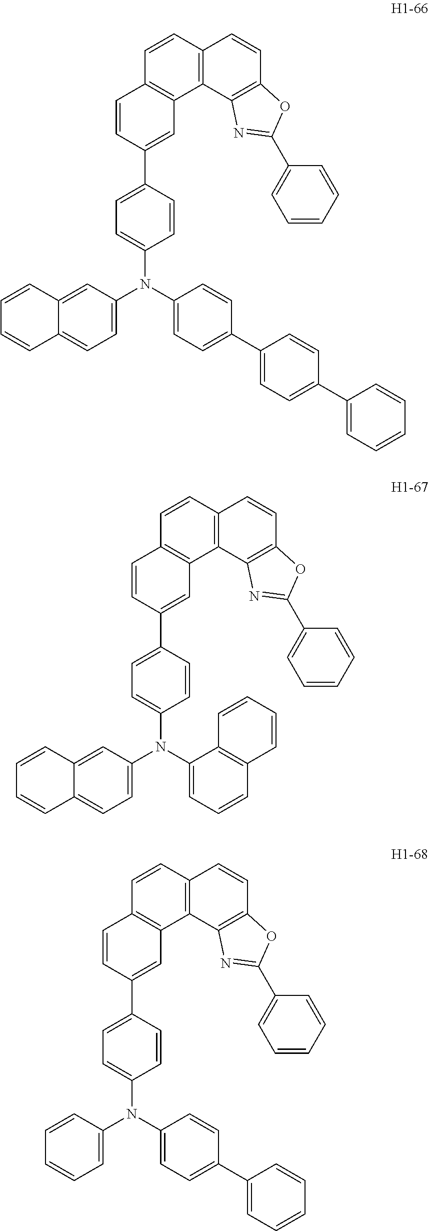 [SMILES: c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1 ; c1ccc(-c2ccc(N(c3ccccc3)c3ccc(-c4ccc5ccc6ccc7oc(-c8ccccc8)nc7c6c5c4)cc3)cc2)cc1]

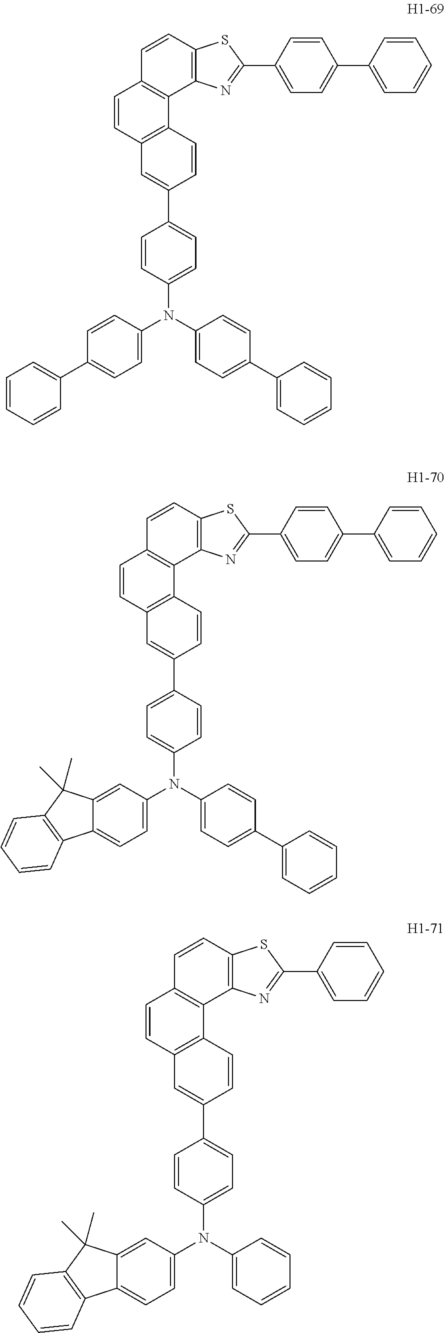 [SMILES: c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21 ; CC1(C)c2ccccc2-c2ccc(N(c3ccccc3)c3ccc(-c4ccc5c(ccc6ccc7sc(-c8ccccc8)nc7c65)c4)cc3)cc21]

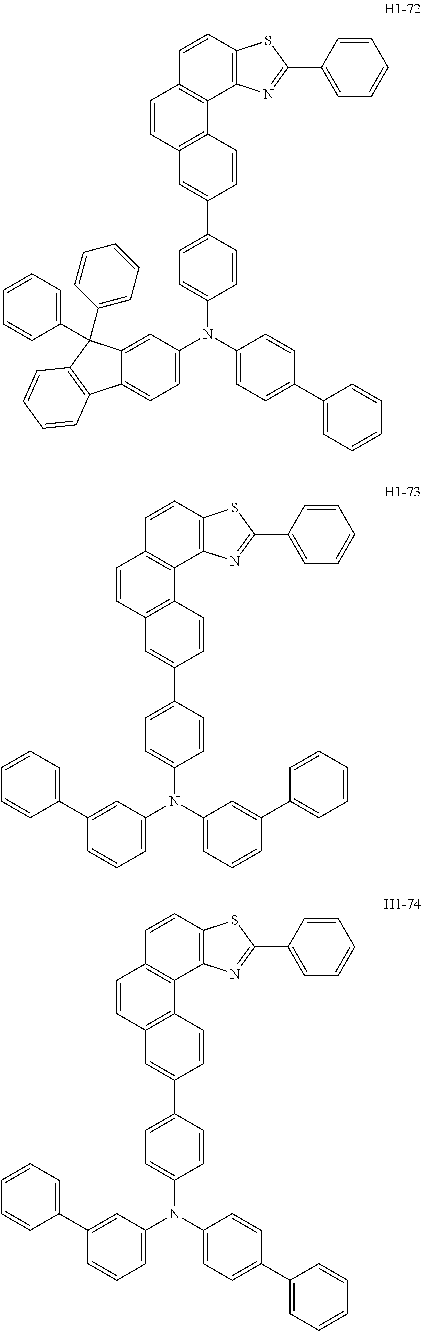 [SMILES: c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1]

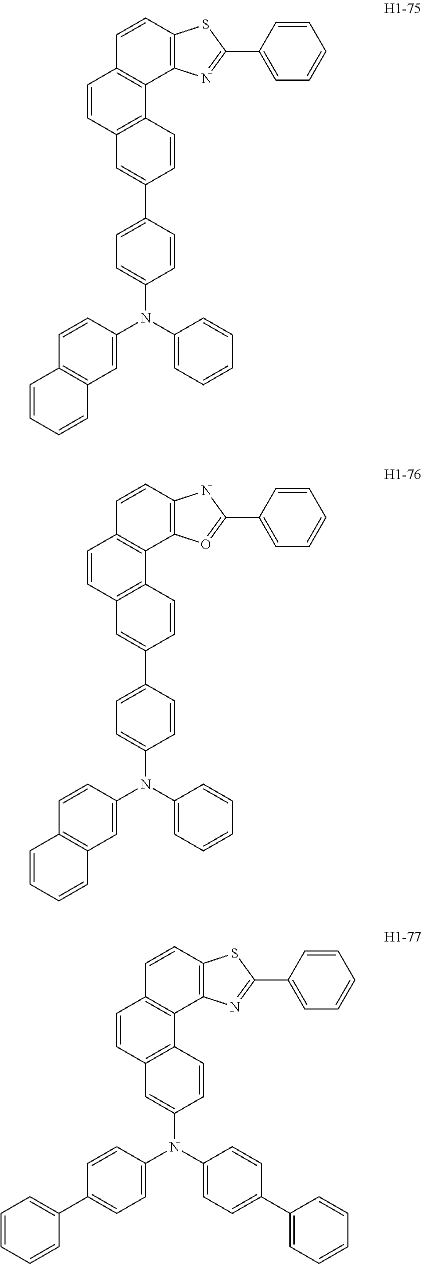

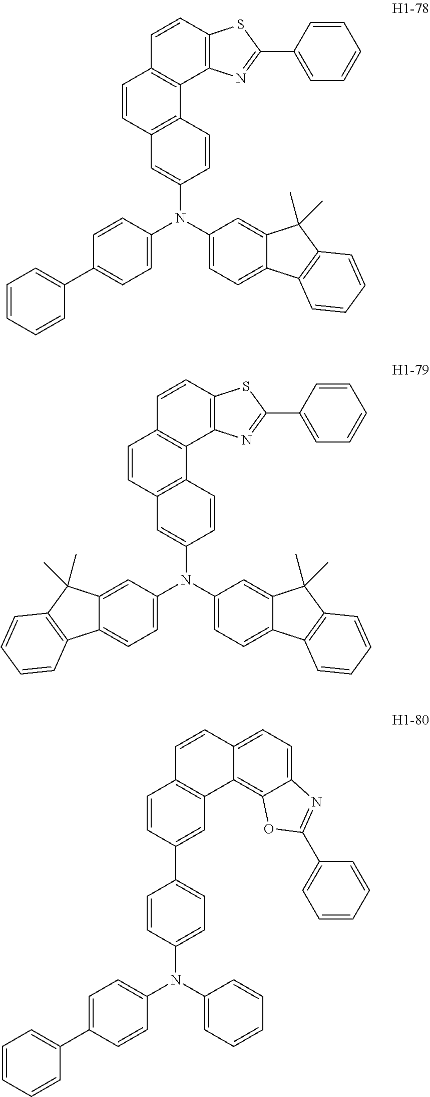 [SMILES: CC1(C)c2ccccc2-c2ccc(N(c3ccc(-c4ccccc4)cc3)c3ccc4c(ccc5ccc6sc(-c7ccccc7)nc6c54)c3)cc21 ; CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; c1ccc(-c2ccc(N(c3ccccc3)c3ccc(-c4ccc5ccc6ccc7nc(-c8ccccc8)oc7c6c5c4)cc3)cc2)cc1]

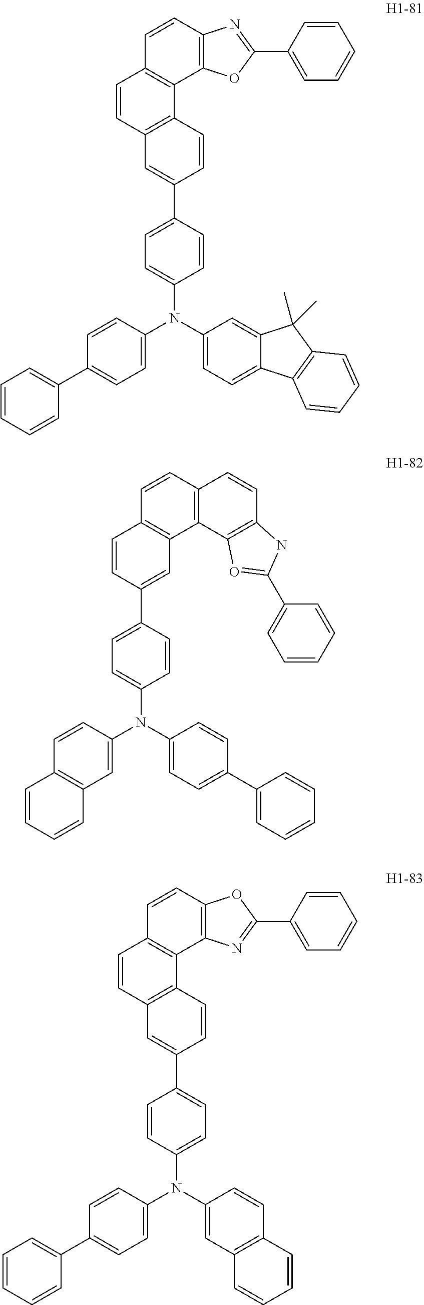

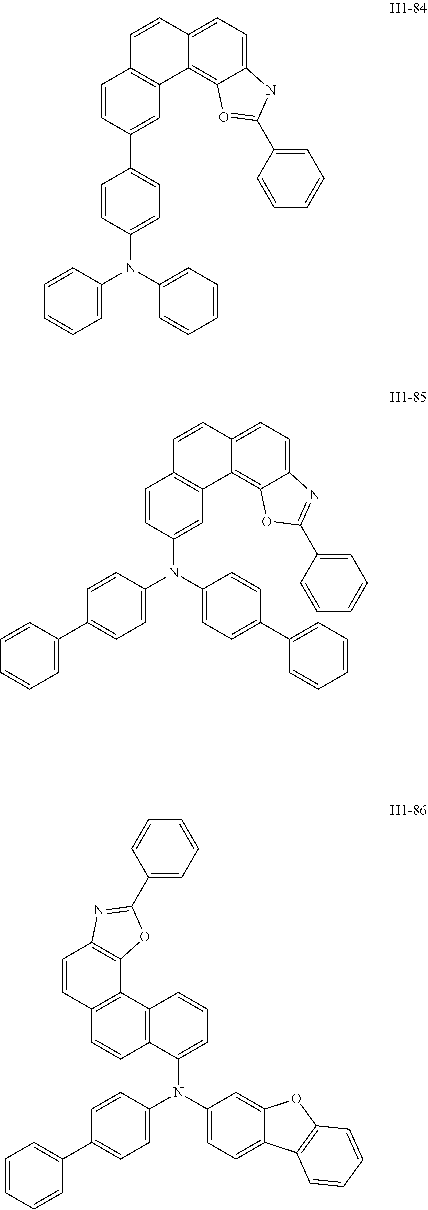

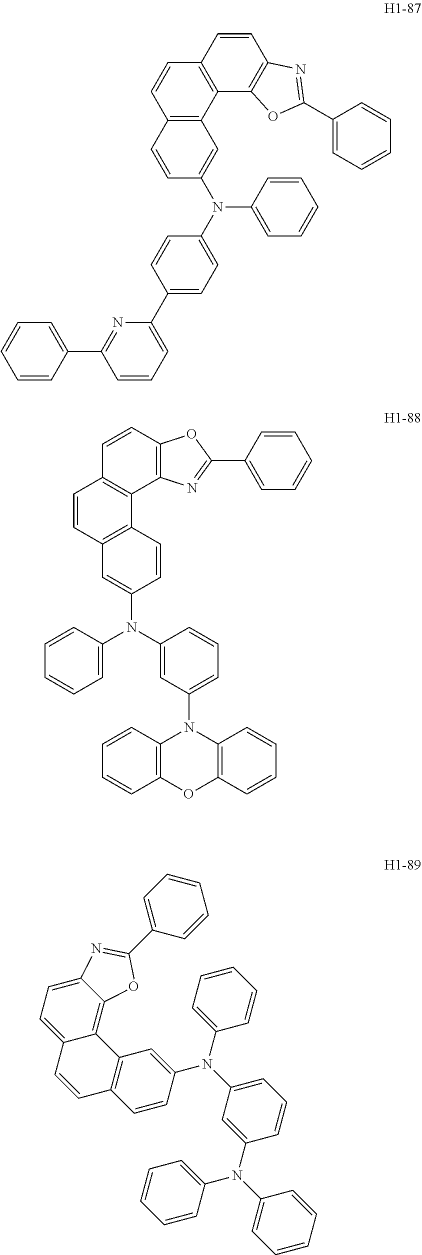 [SMILES: c1ccc(-c2cccc(-c3ccc(N(c4ccccc4)c4ccc5ccc6ccc7nc(-c8ccccc8)oc7c6c5c4)cc3)n2)cc1 ; c1ccc(-c2nc3c(ccc4ccc5cc(N(c6ccccc6)c6cccc(N7c8ccccc8Oc8ccccc87)c6)ccc5c43)o2)cc1 ; c1ccc(-c2nc3ccc4ccc5ccc(N(c6ccccc6)c6cccc(N(c7ccccc7)c7ccccc7)c6)cc5c4c3o2)cc1]

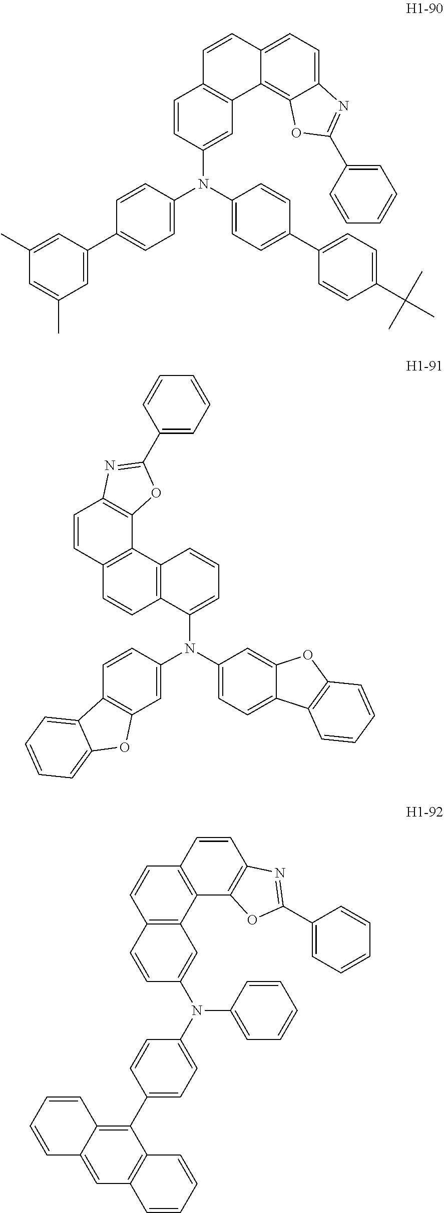 [SMILES: Cc1cc(C)cc(-c2ccc(N(c3ccc(-c4ccc(C(C)(C)C)cc4)cc3)c3ccc4ccc5ccc6nc(-c7ccccc7)oc6c5c4c3)cc2)c1 ; c1ccc(-c2nc3ccc4ccc5c(N(c6ccc7c(c6)oc6ccccc67)c6ccc7c(c6)oc6ccccc67)cccc5c4c3o2)cc1 ; c1ccc(-c2nc3ccc4ccc5ccc(N(c6ccccc6)c6ccc(-c7c8ccccc8cc8ccccc78)cc6)cc5c4c3o2)cc1]

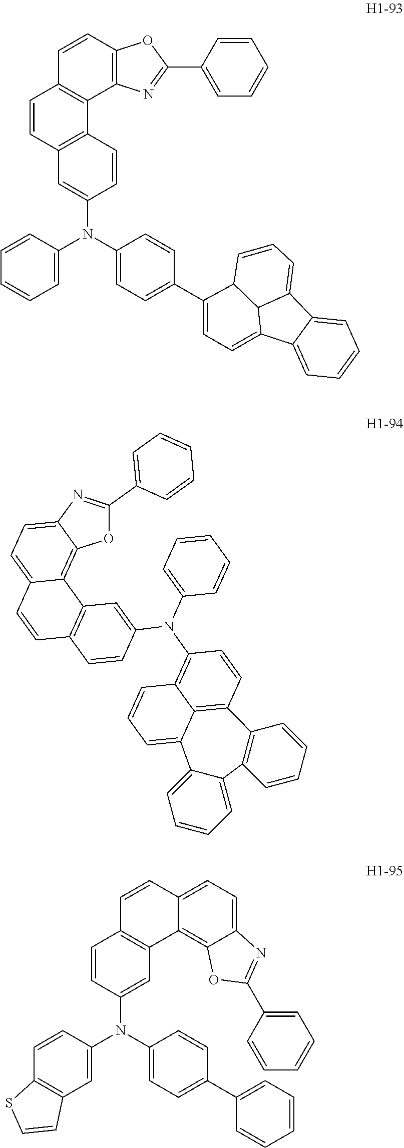

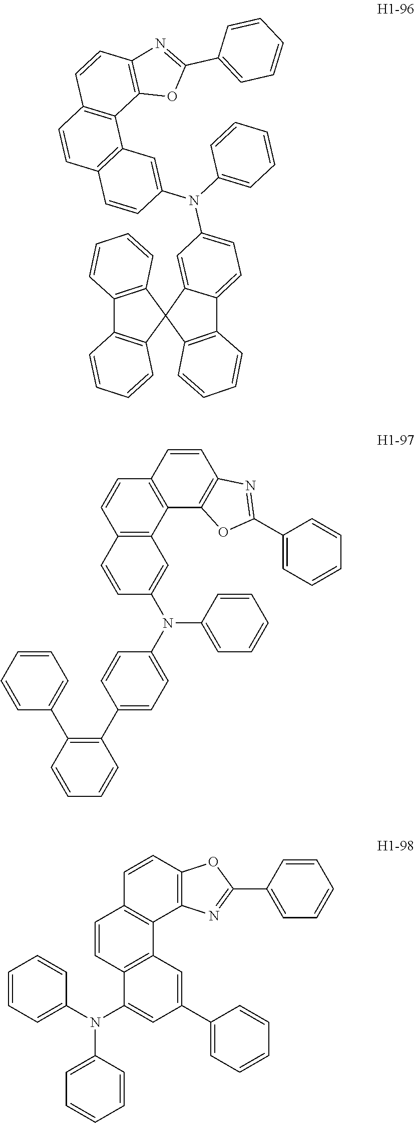 [SMILES: c1ccc(-c2nc3ccc4ccc5ccc(N(c6ccccc6)c6ccc7c(c6)C6(c8ccccc8-c8ccccc86)c6ccccc6-7)cc5c4c3o2)cc1 ; c1ccc(-c2nc3ccc4ccc5ccc(N(c6ccccc6)c6ccc(-c7ccccc7-c7ccccc7)cc6)cc5c4c3o2)cc1 ; c1ccc(-c2cc(N(c3ccccc3)c3ccccc3)c3ccc4ccc5oc(-c6ccccc6)nc5c4c3c2)cc1]

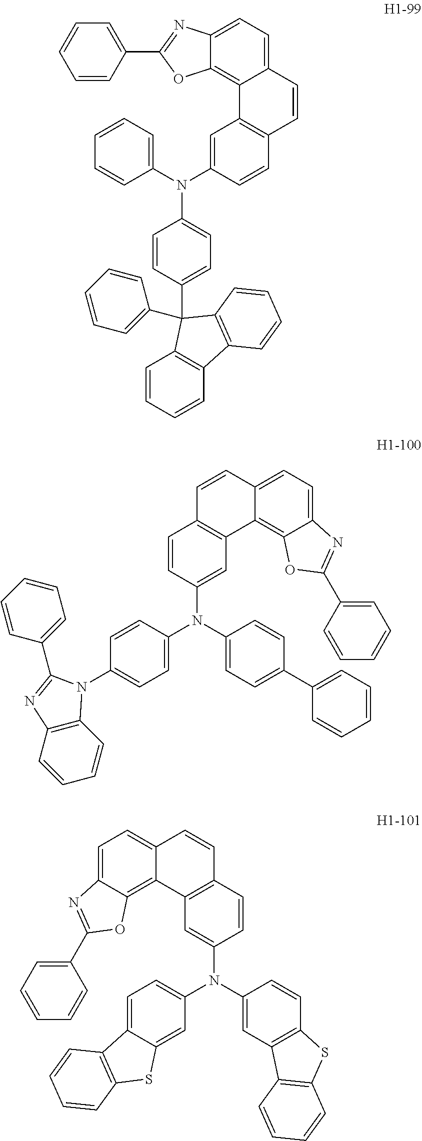 [SMILES: c1ccc(-c2nc3ccc4ccc5ccc(N(c6ccccc6)c6ccc(C7(c8ccccc8)c8ccccc8-c8ccccc87)cc6)cc5c4c3o2)cc1 ; c1ccc(-c2ccc(N(c3ccc(-n4c(-c5ccccc5)nc5ccccc54)cc3)c3ccc4ccc5ccc6nc(-c7ccccc7)oc6c5c4c3)cc2)cc1 ; c1ccc(-c2nc3ccc4ccc5ccc(N(c6ccc7c(c6)sc6ccccc67)c6ccc7sc8ccccc8c7c6)cc5c4c3o2)cc1]

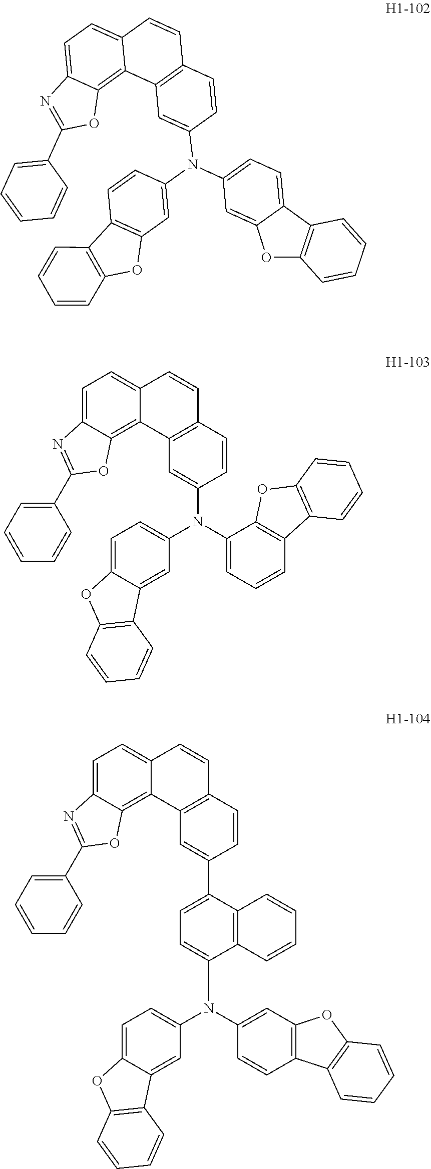 [SMILES: c1ccc(-c2nc3ccc4ccc5ccc(N(c6ccc7c(c6)oc6ccccc67)c6ccc7c(c6)oc6ccccc67)cc5c4c3o2)cc1 ; c1ccc(-c2nc3ccc4ccc5ccc(N(c6ccc7oc8ccccc8c7c6)c6cccc7c6oc6ccccc67)cc5c4c3o2)cc1 ; c1ccc(-c2nc3ccc4ccc5ccc(-c6ccc(N(c7ccc8c(c7)oc7ccccc78)c7ccc8oc9ccccc9c8c7)c7ccccc67)cc5c4c3o2)cc1]

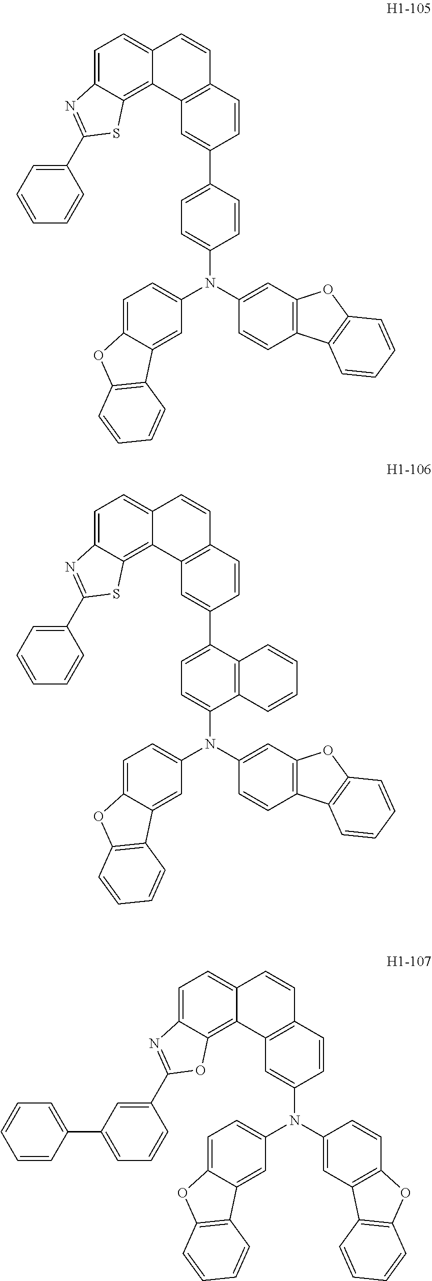 [SMILES: c1ccc(-c2nc3ccc4ccc5ccc(-c6ccc(N(c7ccc8c(c7)oc7ccccc78)c7ccc8oc9ccccc9c8c7)cc6)cc5c4c3s2)cc1 ; c1ccc(-c2nc3ccc4ccc5ccc(-c6ccc(N(c7ccc8c(c7)oc7ccccc78)c7ccc8oc9ccccc9c8c7)c7ccccc67)cc5c4c3s2)cc1 ; c1ccc(-c2cccc(-c3nc4ccc5ccc6ccc(N(c7ccc8oc9ccccc9c8c7)c7ccc8oc9ccccc9c8c7)cc6c5c4o3)c2)cc1]

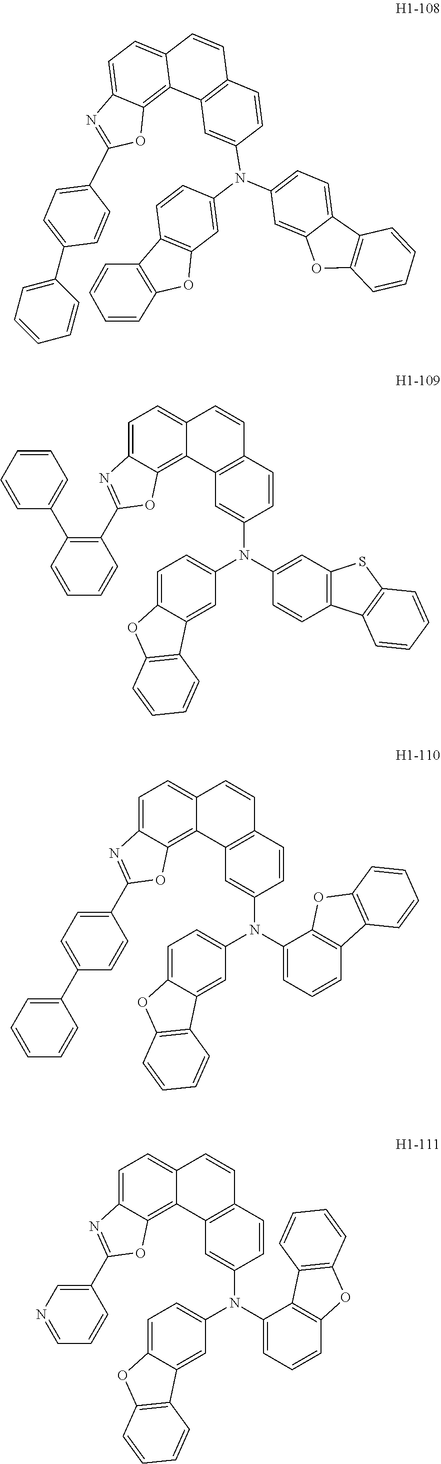 [SMILES: c1ccc(-c2ccc(-c3nc4ccc5ccc6ccc(N(c7ccc8c(c7)oc7ccccc78)c7ccc8c(c7)oc7ccccc78)cc6c5c4o3)cc2)cc1 ; c1ccc(-c2ccccc2-c2nc3ccc4ccc5ccc(N(c6ccc7c(c6)sc6ccccc67)c6ccc7oc8ccccc8c7c6)cc5c4c3o2)cc1 ; c1ccc(-c2ccc(-c3nc4ccc5ccc6ccc(N(c7ccc8oc9ccccc9c8c7)c7cccc8c7oc7ccccc78)cc6c5c4o3)cc2)cc1 ; c1cncc(-c2nc3ccc4ccc5ccc(N(c6ccc7oc8ccccc8c7c6)c6cccc7oc8ccccc8c67)cc5c4c3o2)c1]

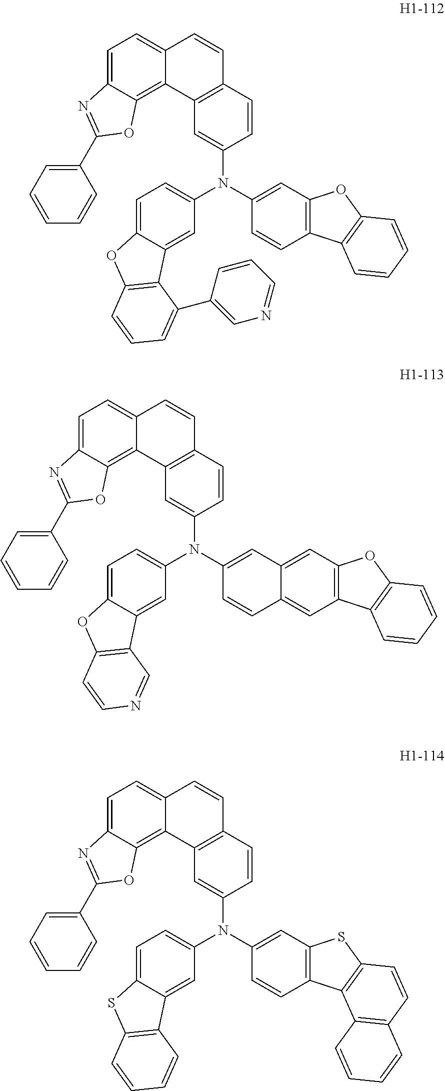 [SMILES: c1ccc(-c2nc3ccc4ccc5ccc(N(c6ccc7c(c6)oc6ccccc67)c6ccc7oc8cccc(-c9cccnc9)c8c7c6)cc5c4c3o2)cc1 ; c1ccc(-c2nc3ccc4ccc5ccc(N(c6ccc7cc8c(cc7c6)oc6ccccc68)c6ccc7oc8ccncc8c7c6)cc5c4c3o2)cc1 ; c1ccc(-c2nc3ccc4ccc5ccc(N(c6ccc7c(c6)sc6ccc8ccccc8c67)c6ccc7sc8ccccc8c7c6)cc5c4c3o2)cc1]

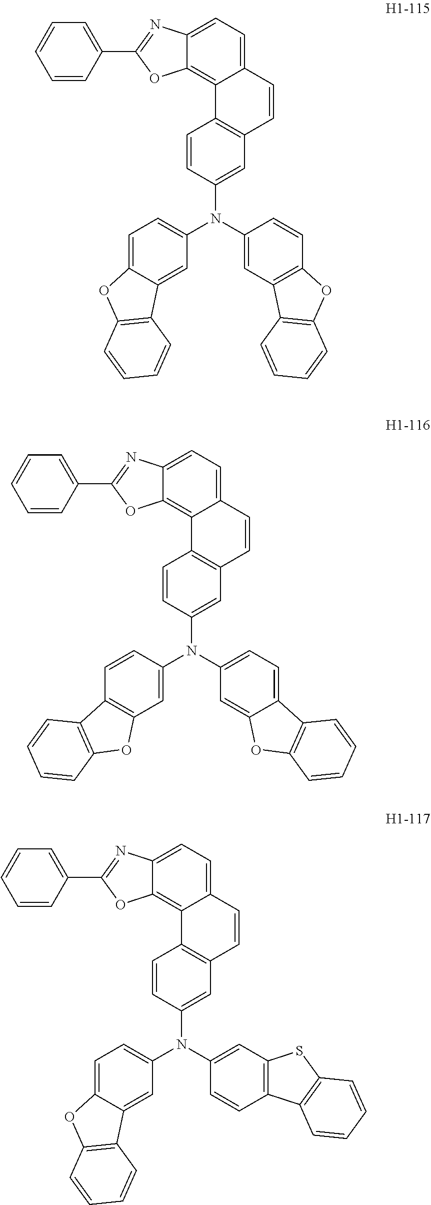 [SMILES: c1ccc(-c2nc3ccc4ccc5cc(N(c6ccc7oc8ccccc8c7c6)c6ccc7oc8ccccc8c7c6)ccc5c4c3o2)cc1 ; c1ccc(-c2nc3ccc4ccc5cc(N(c6ccc7c(c6)oc6ccccc67)c6ccc7c(c6)oc6ccccc67)ccc5c4c3o2)cc1 ; c1ccc(-c2nc3ccc4ccc5cc(N(c6ccc7c(c6)sc6ccccc67)c6ccc7oc8ccccc8c7c6)ccc5c4c3o2)cc1]

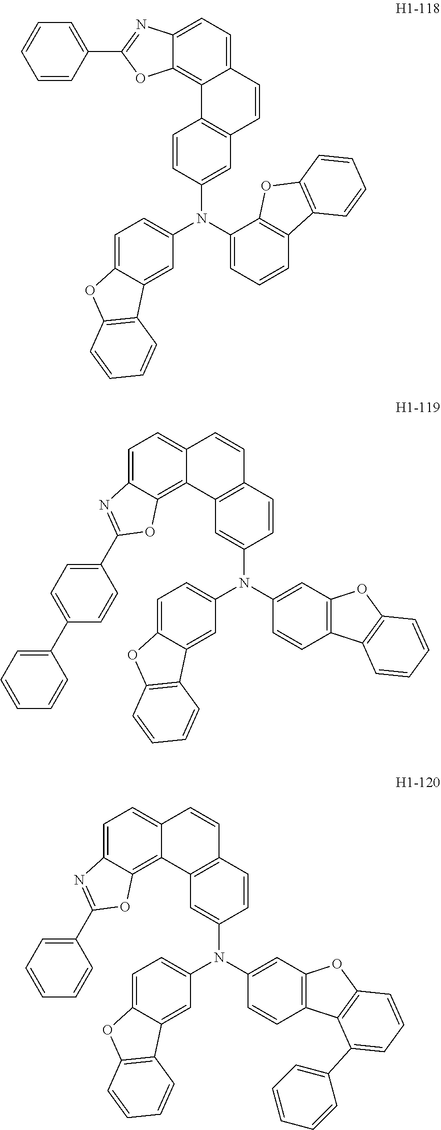 [SMILES: c1ccc(-c2nc3ccc4ccc5cc(N(c6ccc7oc8ccccc8c7c6)c6cccc7c6oc6ccccc67)ccc5c4c3o2)cc1 ; c1ccc(-c2ccc(-c3nc4ccc5ccc6ccc(N(c7ccc8c(c7)oc7ccccc78)c7ccc8oc9ccccc9c8c7)cc6c5c4o3)cc2)cc1 ; c1ccc(-c2nc3ccc4ccc5ccc(N(c6ccc7c(c6)oc6cccc(-c8ccccc8)c67)c6ccc7oc8ccccc8c7c6)cc5c4c3o2)cc1]

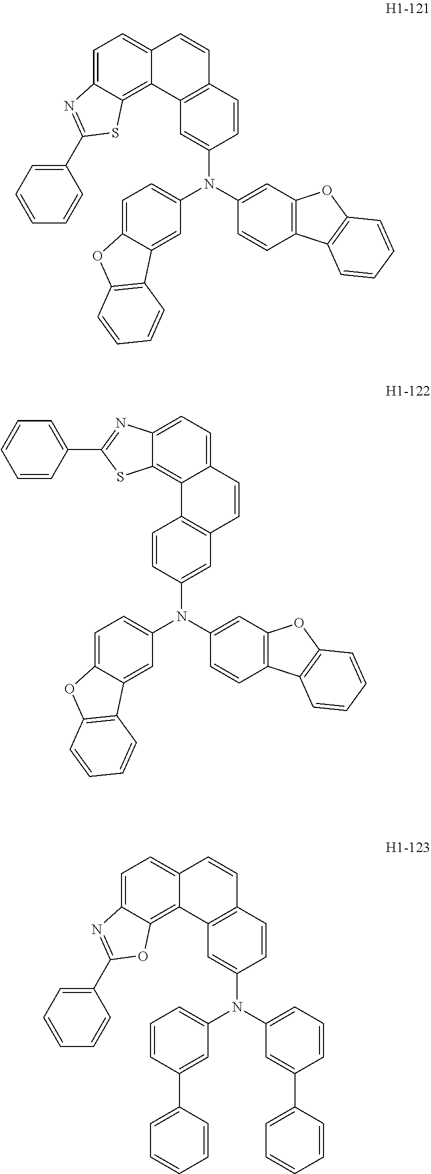 [SMILES: c1ccc(-c2nc3ccc4ccc5ccc(N(c6ccc7c(c6)oc6ccccc67)c6ccc7oc8ccccc8c7c6)cc5c4c3s2)cc1 ; c1ccc(-c2nc3ccc4ccc5cc(N(c6ccc7c(c6)oc6ccccc67)c6ccc7oc8ccccc8c7c6)ccc5c4c3s2)cc1 ; c1ccc(-c2cccc(N(c3cccc(-c4ccccc4)c3)c3ccc4ccc5ccc6nc(-c7ccccc7)oc6c5c4c3)c2)cc1]

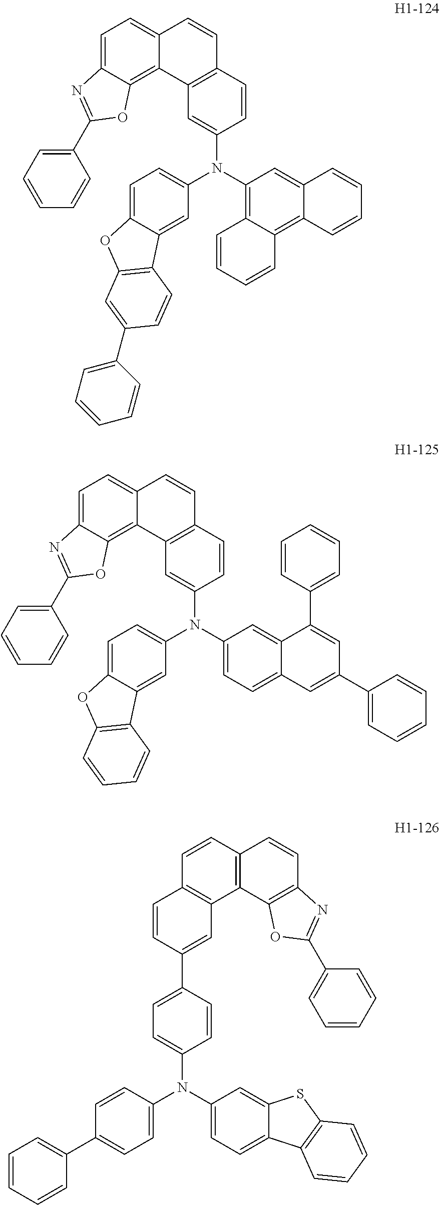 [SMILES: c1ccc(-c2ccc3c(c2)oc2ccc(N(c4ccc5ccc6ccc7nc(-c8ccccc8)oc7c6c5c4)c4cc5ccccc5c5ccccc45)cc23)cc1 ; c1ccc(-c2cc(-c3ccccc3)c3cc(N(c4ccc5oc6ccccc6c5c4)c4ccc5ccc6ccc7nc(-c8ccccc8)oc7c6c5c4)ccc3c2)cc1 ; c1ccc(-c2ccc(N(c3ccc(-c4ccc5ccc6ccc7nc(-c8ccccc8)oc7c6c5c4)cc3)c3ccc4c(c3)sc3ccccc34)cc2)cc1]

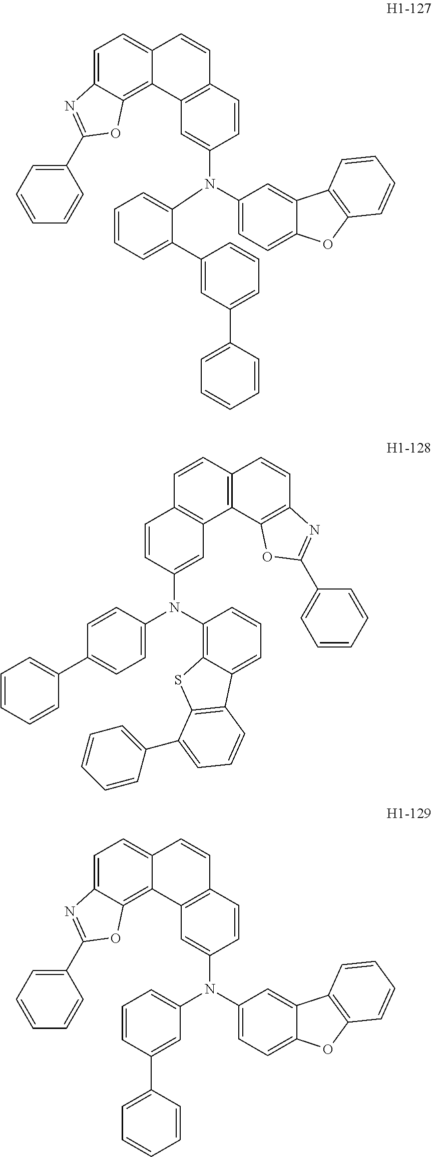 [SMILES: c1ccc(-c2cccc(-c3ccccc3N(c3ccc4oc5ccccc5c4c3)c3ccc4ccc5ccc6nc(-c7ccccc7)oc6c5c4c3)c2)cc1 ; c1ccc(-c2ccc(N(c3ccc4ccc5ccc6nc(-c7ccccc7)oc6c5c4c3)c3cccc4c3sc3c(-c5ccccc5)cccc34)cc2)cc1 ; c1ccc(-c2cccc(N(c3ccc4oc5ccccc5c4c3)c3ccc4ccc5ccc6nc(-c7ccccc7)oc6c5c4c3)c2)cc1]

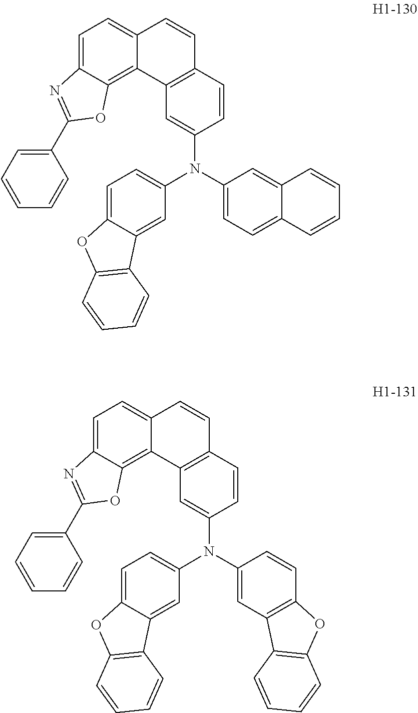 [SMILES: c1ccc(-c2nc3ccc4ccc5ccc(N(c6ccc7ccccc7c6)c6ccc7oc8ccccc8c7c6)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1]

The compound represented by the formula 1 according to one embodiment may be produced with reference to a synthetic method known to a person skilled in the art.

The second host material according to another embodiment comprises an organic electroluminescent compound represented by the following formula 3.

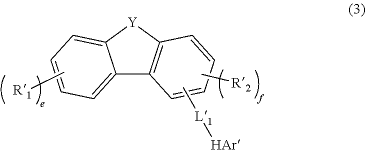

In formula 3.

Y represents —O— or —S—;

L′₁ represents a single bond, a substituted or unsubstituted (C6-C30)arylene, or a substituted or unsubstituted (3- to 30-membered)heteroarylene;

HAr′ represents a substituted or unsubstituted (3- to 30-membered)heteroaryl comprising at least one nitrogen;

R′₁ and R′ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or may be linked to adjacent substituents to form a ring(s); and

e represents an integer of 1 to 4, and f represent an integer of 1 to 3; and when e and f are an integer of 2 or more, each of R′₁ and each of R′₂ may be the same or different.

In one embodiment, L′₁ may be a single bond or a substituted or unsubstituted (C6-C30)arylene, preferably a single bond or a substituted or unsubstituted (C6-C25)arylene, more preferably a single bond or a substituted or unsubstituted (C6-C18)arylene. For example, L′₁ may be a single bond, phenylene unsubstituted or substituted with naphthyl, naphthylene unsubstituted or substituted with phenyl, or an unsubstituted biphenylene.

In one embodiment, HAr′ may be a substituted or unsubstituted (5- to 30-membered)heteroaryl comprising at least one nitrogen, preferably a (5- to 25-membered)heteroaryl comprising at least two nitrogens and unsubstituted or substituted with (C6-C30)aryl and/or (5- to 30-membered)heteroaryl, more preferably a (5- to 18-membered)heteroaryl comprising at least two nitrogens and unsubstituted or substituted with (C6-C25)aryl and/or (5- to 25-membered)heteroaryl. For example, HAr′ may be a substituted or unsubstituted triazinyl, a substituted or unsubstituted quinazolinyl, a substituted or unsubstituted quinoxalinyl, a substituted or unsubstituted naphthyridinyl, a substituted or unsubstituted pyridopyrazinyl, a substituted or unsubstituted benzoquinazolinyl, or a substituted or unsubstituted benzoquinoxalinyl. Wherein, the substituents of the substituted groups may be at least one of a substituted or unsubstituted (C6-C30)aryl and a substituted or unsubstituted (5- to 30-membered)heteroaryl, preferably at least one of a substituted or unsubstituted (C6-C25)aryl and a substituted or unsubstituted (5- to 25-membered)heteroaryl, more preferably at least one of a substituted or unsubstituted (C6-C18)aryl and a substituted or unsubstituted (5- to 18-membered)heteroaryl. For example, the substituents of the substituted groups may be at least one of phenyl unsubstituted or substituted with at least one of naphthyl, m-biphenyl, carbazolyl, dibenzothiophenyl, dibenzofuranyl, and quinoxalinyl substituted with phenyl; biphenyl; terphenyl; naphthyl unsubstituted or substituted with phenyl; carbazolyl unsubstituted or substituted with phenyl; fluorenyl; phenanthrenyl; triphenylenyl; benzofluorenyl; dibenzofuranyl; dibenzothiophenyl; and benzocarbazolyl.

In one embodiment, R′₁ and R′₂ each independently may be hydrogen, deuterium, halogen, cyano, or a substituted or unsubstituted (C1-C30)alkyl. For example, all of R′₁ and R′₂ may be hydrogen.

According to one embodiment, the organic electroluminescent compound of formula 3 may be more specifically illustrated by the following compounds, but is not limited thereto.

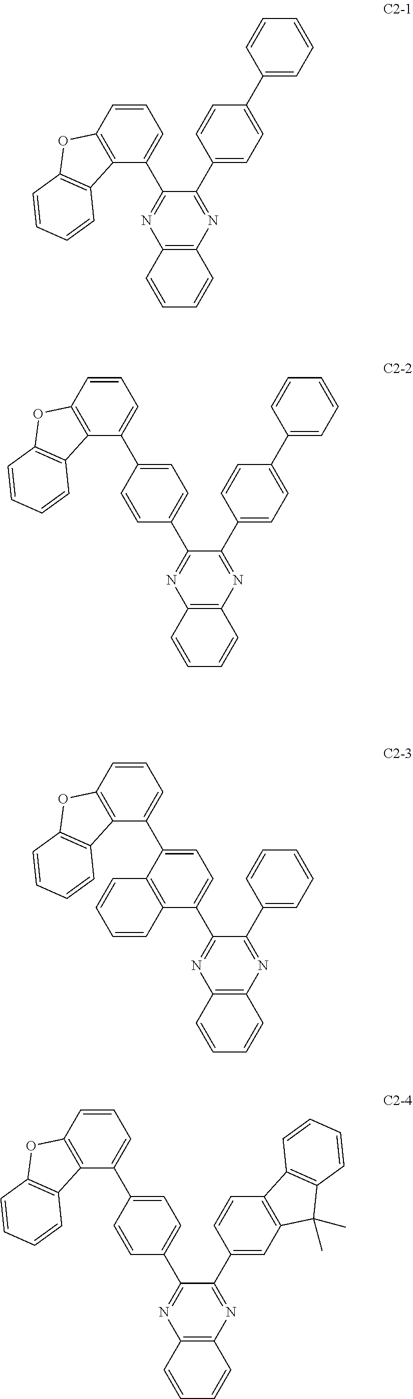 [SMILES: c1ccc(-c2ccc(-c3nc4ccccc4nc3-c3cccc4oc5ccccc5c34)cc2)cc1 ; c1ccc(-c2ccc(-c3nc4ccccc4nc3-c3ccc(-c4cccc5oc6ccccc6c45)cc3)cc2)cc1 ; c1ccc(-c2nc3ccccc3nc2-c2ccc(-c3cccc4oc5ccccc5c34)c3ccccc23)cc1 ; CC1(C)c2ccccc2-c2ccc(-c3nc4ccccc4nc3-c3ccc(-c4cccc5oc6ccccc6c45)cc3)cc21]

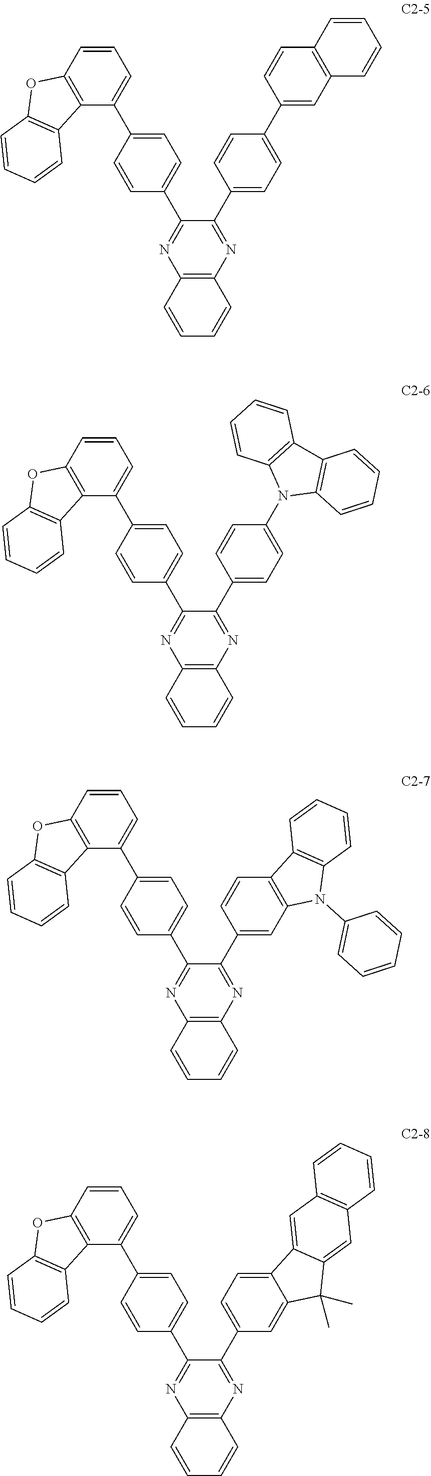 [SMILES: c1ccc2cc(-c3ccc(-c4nc5ccccc5nc4-c4ccc(-c5cccc6oc7ccccc7c56)cc4)cc3)ccc2c1 ; c1ccc2nc(-c3ccc(-n4c5ccccc5c5ccccc54)cc3)c(-c3ccc(-c4cccc5oc6ccccc6c45)cc3)nc2c1 ; c1ccc(-n2c3ccccc3c3ccc(-c4nc5ccccc5nc4-c4ccc(-c5cccc6oc7ccccc7c56)cc4)cc32)cc1 ; CC1(C)c2cc(-c3nc4ccccc4nc3-c3ccc(-c4cccc5oc6ccccc6c45)cc3)ccc2-c2cc3ccccc3cc21]

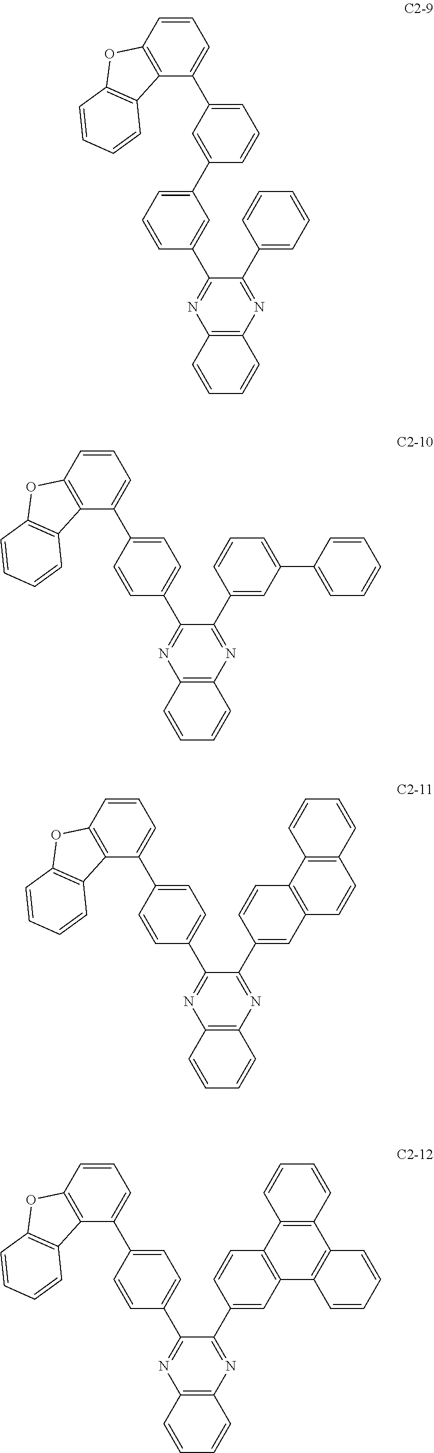 [SMILES: c1ccc(-c2nc3ccccc3nc2-c2cccc(-c3cccc(-c4cccc5oc6ccccc6c45)c3)c2)cc1 ; c1ccc(-c2cccc(-c3nc4ccccc4nc3-c3ccc(-c4cccc5oc6ccccc6c45)cc3)c2)cc1 ; c1ccc2c(c1)ccc1cc(-c3nc4ccccc4nc3-c3ccc(-c4cccc5oc6ccccc6c45)cc3)ccc12 ; c1ccc2nc(-c3ccc4c5ccccc5c5ccccc5c4c3)c(-c3ccc(-c4cccc5oc6ccccc6c45)cc3)nc2c1]

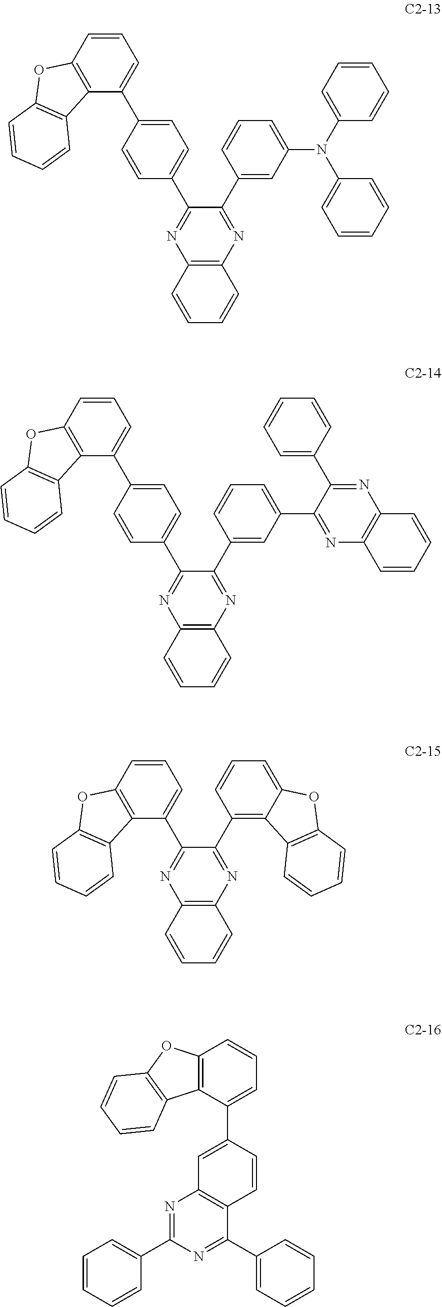 [SMILES: c1ccc(N(c2ccccc2)c2cccc(-c3nc4ccccc4nc3-c3ccc(-c4cccc5oc6ccccc6c45)cc3)c2)cc1 ; c1ccc(-c2nc3ccccc3nc2-c2cccc(-c3nc4ccccc4nc3-c3ccc(-c4cccc5oc6ccccc6c45)cc3)c2)cc1 ; c1ccc2nc(-c3cccc4oc5ccccc5c34)c(-c3cccc4oc5ccccc5c34)nc2c1 ; c1ccc(-c2nc(-c3ccccc3)c3ccc(-c4cccc5oc6ccccc6c45)cc3n2)cc1]

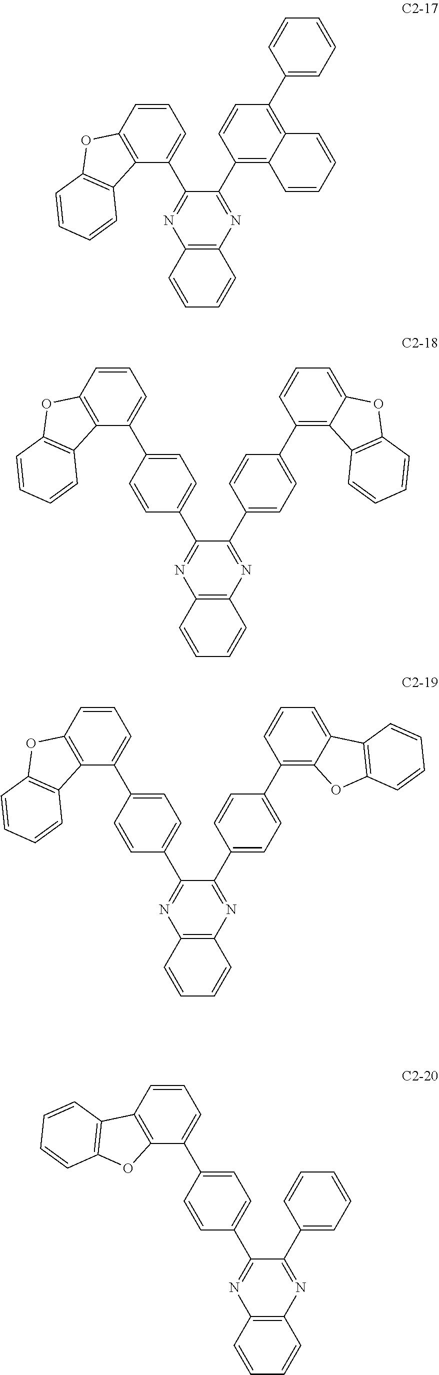 [SMILES: c1ccc(-c2ccc(-c3nc4ccccc4nc3-c3cccc4oc5ccccc5c34)c3ccccc23)cc1 ; c1ccc2nc(-c3ccc(-c4cccc5oc6ccccc6c45)cc3)c(-c3ccc(-c4cccc5oc6ccccc6c45)cc3)nc2c1 ; c1ccc2nc(-c3ccc(-c4cccc5oc6ccccc6c45)cc3)c(-c3ccc(-c4cccc5c4oc4ccccc45)cc3)nc2c1 ; c1ccc(-c2nc3ccccc3nc2-c2ccc(-c3cccc4c3oc3ccccc34)cc2)cc1]

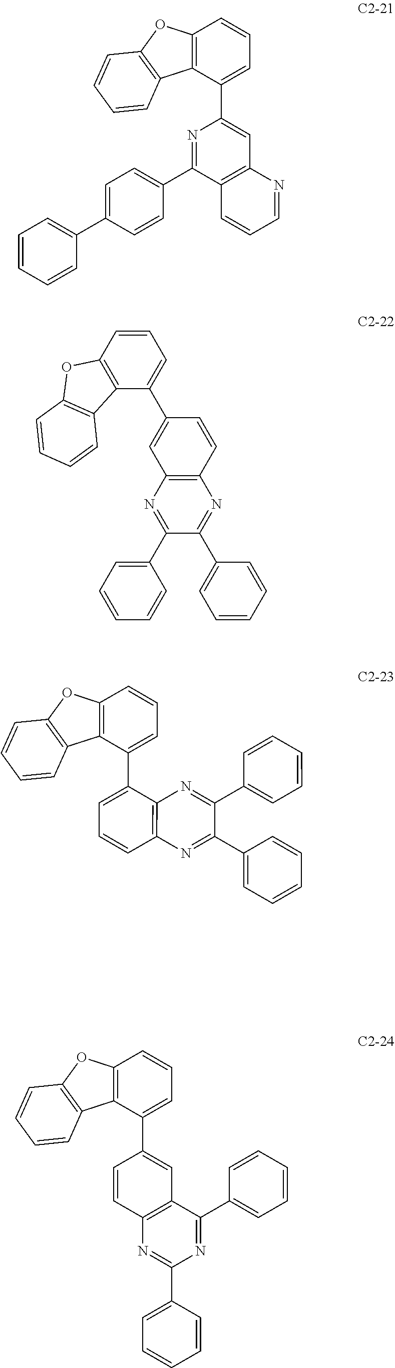 [SMILES: c1ccc(-c2ccc(-c3nc(-c4cccc5oc6ccccc6c45)cc4ncccc34)cc2)cc1 ; c1ccc(-c2nc3ccc(-c4cccc5oc6ccccc6c45)cc3nc2-c2ccccc2)cc1 ; c1ccc(-c2nc3cccc(-c4cccc5oc6ccccc6c45)c3nc2-c2ccccc2)cc1 ; c1ccc(-c2nc(-c3ccccc3)c3cc(-c4cccc5oc6ccccc6c45)ccc3n2)cc1]

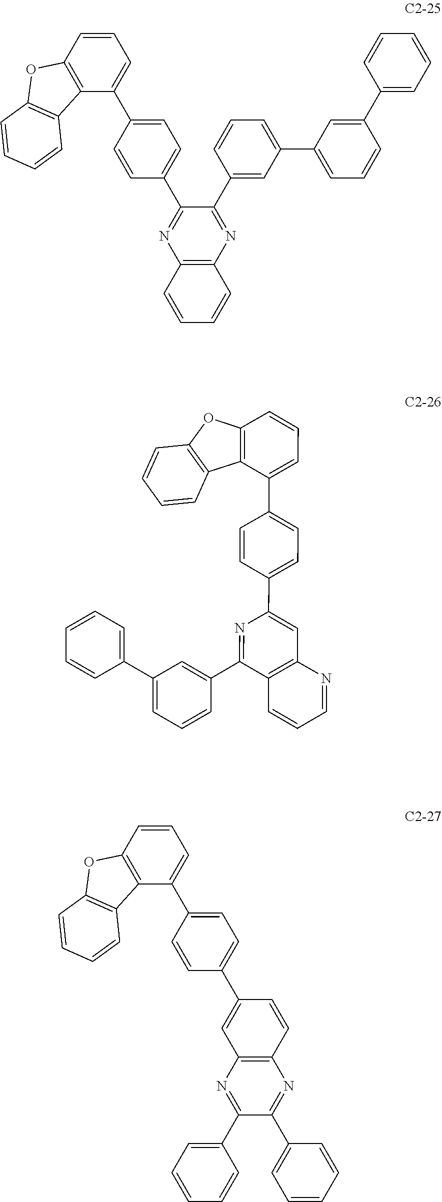 [SMILES: c1ccc(-c2cccc(-c3cccc(-c4nc5ccccc5nc4-c4ccc(-c5cccc6oc7ccccc7c56)cc4)c3)c2)cc1 ; c1ccc(-c2cccc(-c3nc(-c4ccc(-c5cccc6oc7ccccc7c56)cc4)cc4ncccc34)c2)cc1 ; c1ccc(-c2nc3ccc(-c4ccc(-c5cccc6oc7ccccc7c56)cc4)cc3nc2-c2ccccc2)cc1]

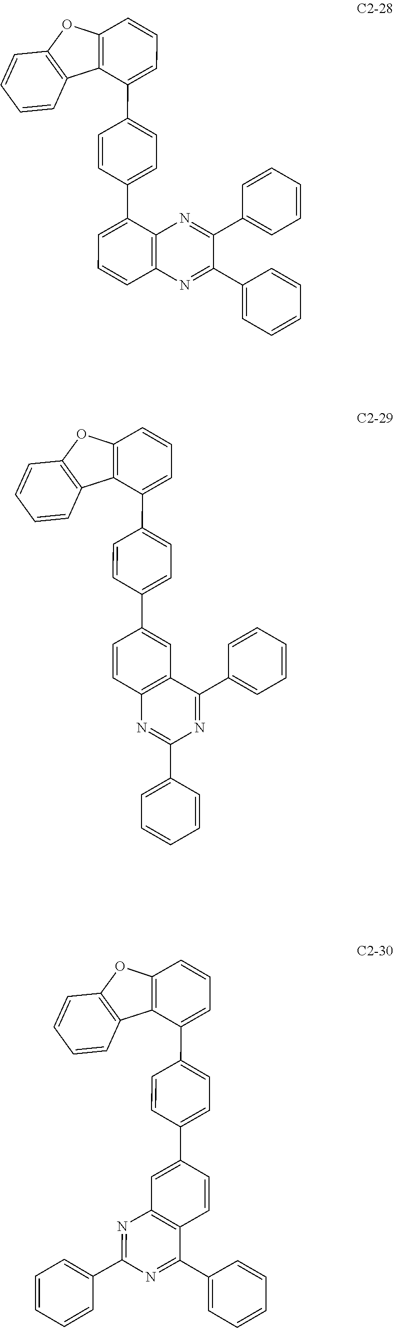 [SMILES: c1ccc(-c2nc3cccc(-c4ccc(-c5cccc6oc7ccccc7c56)cc4)c3nc2-c2ccccc2)cc1 ; c1ccc(-c2nc(-c3ccccc3)c3cc(-c4ccc(-c5cccc6oc7ccccc7c56)cc4)ccc3n2)cc1 ; c1ccc(-c2nc(-c3ccccc3)c3ccc(-c4ccc(-c5cccc6oc7ccccc7c56)cc4)cc3n2)cc1]

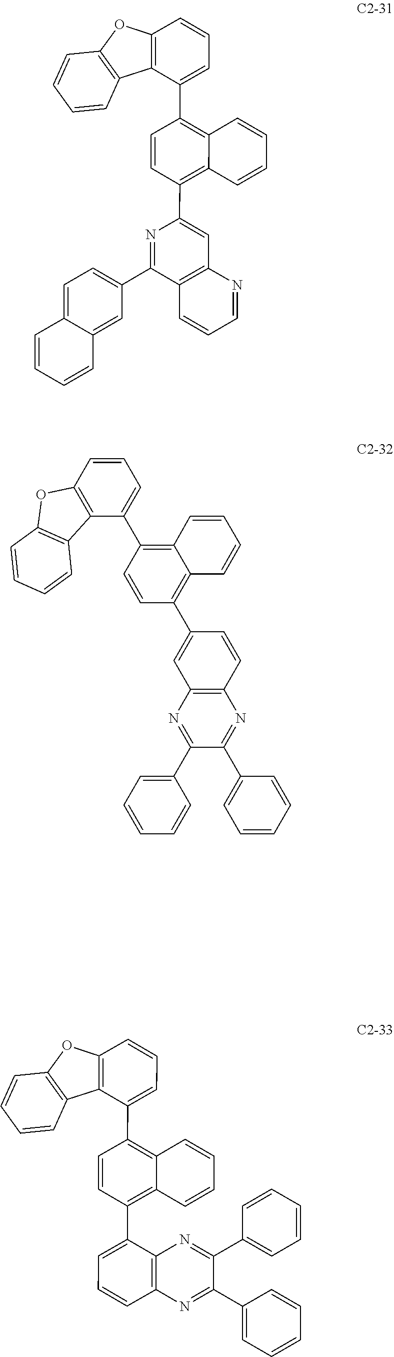 [SMILES: c1ccc2cc(-c3nc(-c4ccc(-c5cccc6oc7ccccc7c56)c5ccccc45)cc4ncccc34)ccc2c1 ; c1ccc(-c2nc3ccc(-c4ccc(-c5cccc6oc7ccccc7c56)c5ccccc45)cc3nc2-c2ccccc2)cc1 ; c1ccc(-c2nc3cccc(-c4ccc(-c5cccc6oc7ccccc7c56)c5ccccc45)c3nc2-c2ccccc2)cc1]

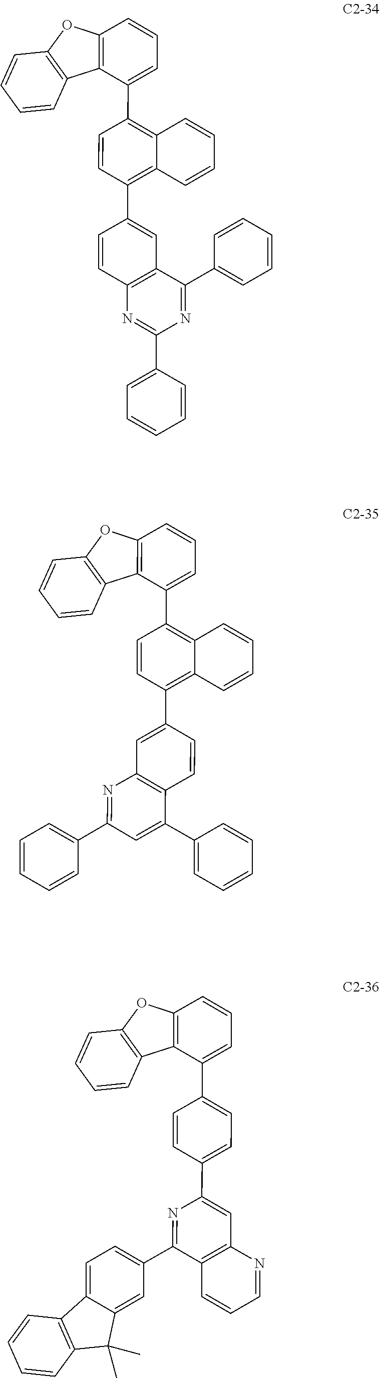 [SMILES: c1ccc(-c2nc(-c3ccccc3)c3cc(-c4ccc(-c5cccc6oc7ccccc7c56)c5ccccc45)ccc3n2)cc1 ; c1ccc(-c2cc(-c3ccccc3)c3ccc(-c4ccc(-c5cccc6oc7ccccc7c56)c5ccccc45)cc3n2)cc1 ; CC1(C)c2ccccc2-c2ccc(-c3nc(-c4ccc(-c5cccc6oc7ccccc7c56)cc4)cc4ncccc34)cc21]

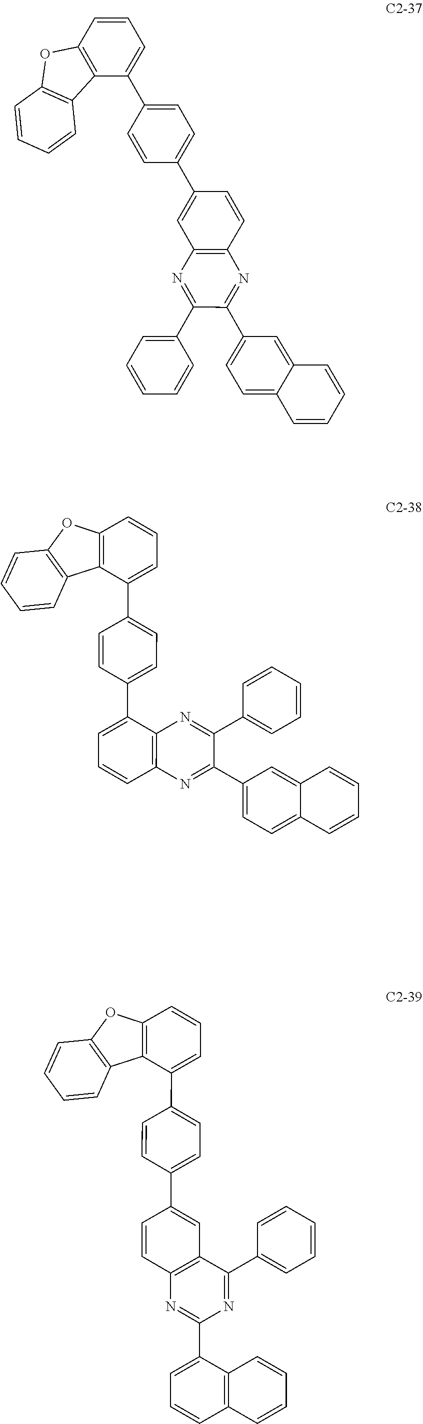 [SMILES: c1ccc(-c2nc3cc(-c4ccc(-c5cccc6oc7ccccc7c56)cc4)ccc3nc2-c2ccc3ccccc3c2)cc1 ; c1ccc(-c2nc3c(-c4ccc(-c5cccc6oc7ccccc7c56)cc4)cccc3nc2-c2ccc3ccccc3c2)cc1 ; c1ccc(-c2nc(-c3cccc4ccccc34)nc3ccc(-c4ccc(-c5cccc6oc7ccccc7c56)cc4)cc23)cc1]

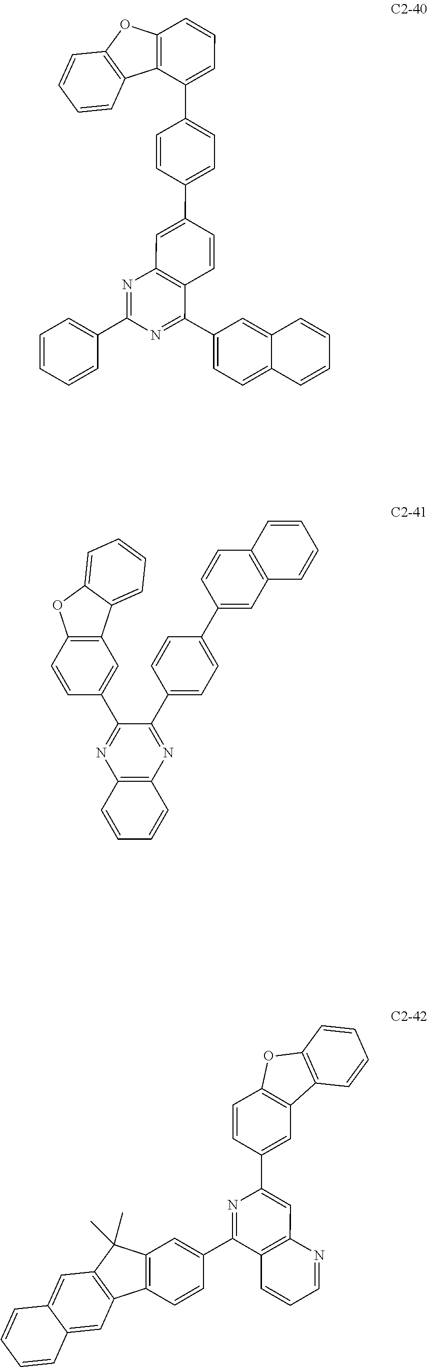 [SMILES: c1ccc(-c2nc(-c3ccc4ccccc4c3)c3ccc(-c4ccc(-c5cccc6oc7ccccc7c56)cc4)cc3n2)cc1 ; c1ccc2cc(-c3ccc(-c4nc5ccccc5nc4-c4ccc5oc6ccccc6c5c4)cc3)ccc2c1 ; CC1(C)c2cc(-c3nc(-c4ccc5oc6ccccc6c5c4)cc4ncccc34)ccc2-c2cc3ccccc3cc21]

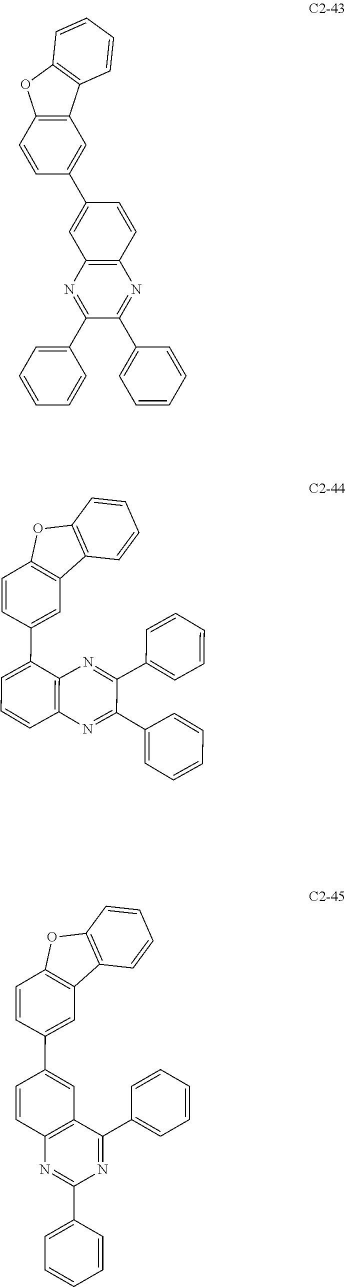 [SMILES: c1ccc(-c2nc3ccc(-c4ccc5oc6ccccc6c5c4)cc3nc2-c2ccccc2)cc1 ; c1ccc(-c2nc3cccc(-c4ccc5oc6ccccc6c5c4)c3nc2-c2ccccc2)cc1 ; c1ccc(-c2nc(-c3ccccc3)c3cc(-c4ccc5oc6ccccc6c5c4)ccc3n2)cc1]

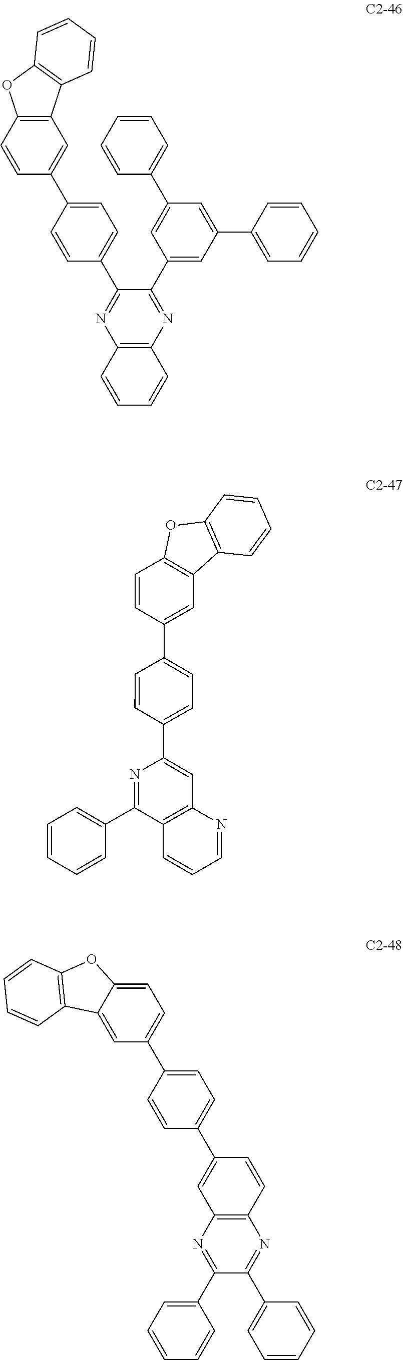 [SMILES: c1ccc(-c2cc(-c3ccccc3)cc(-c3nc4ccccc4nc3-c3ccc(-c4ccc5oc6ccccc6c5c4)cc3)c2)cc1 ; c1ccc(-c2nc(-c3ccc(-c4ccc5oc6ccccc6c5c4)cc3)cc3ncccc23)cc1 ; c1ccc(-c2nc3ccc(-c4ccc(-c5ccc6oc7ccccc7c6c5)cc4)cc3nc2-c2ccccc2)cc1]

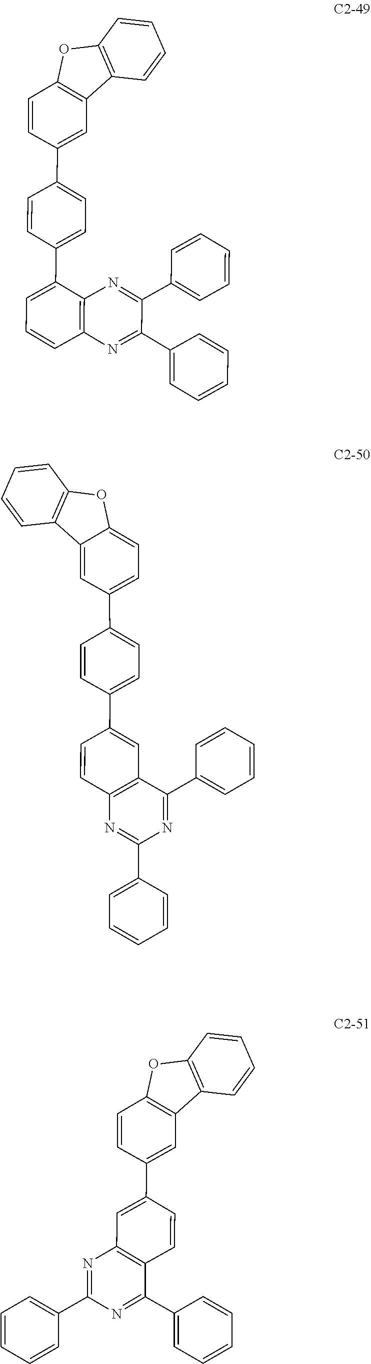 [SMILES: c1ccc(-c2nc3cccc(-c4ccc(-c5ccc6oc7ccccc7c6c5)cc4)c3nc2-c2ccccc2)cc1 ; c1ccc(-c2nc(-c3ccccc3)c3cc(-c4ccc(-c5ccc6oc7ccccc7c6c5)cc4)ccc3n2)cc1 ; c1ccc(-c2nc(-c3ccccc3)c3ccc(-c4ccc5oc6ccccc6c5c4)cc3n2)cc1]

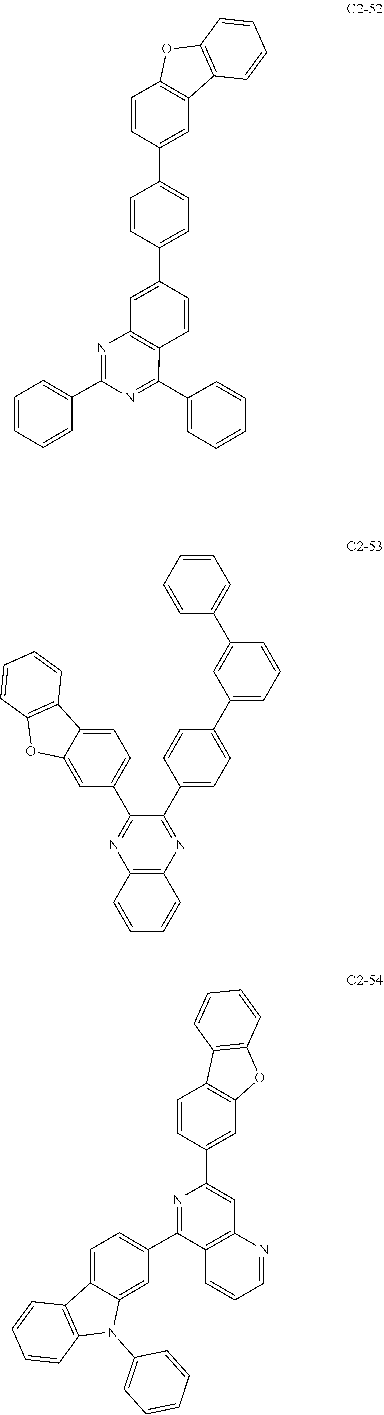 [SMILES: c1ccc(-c2nc(-c3ccccc3)c3ccc(-c4ccc(-c5ccc6oc7ccccc7c6c5)cc4)cc3n2)cc1 ; c1ccc(-c2cccc(-c3ccc(-c4nc5ccccc5nc4-c4ccc5c(c4)oc4ccccc45)cc3)c2)cc1 ; c1ccc(-n2c3ccccc3c3ccc(-c4nc(-c5ccc6c(c5)oc5ccccc56)cc5ncccc45)cc32)cc1]

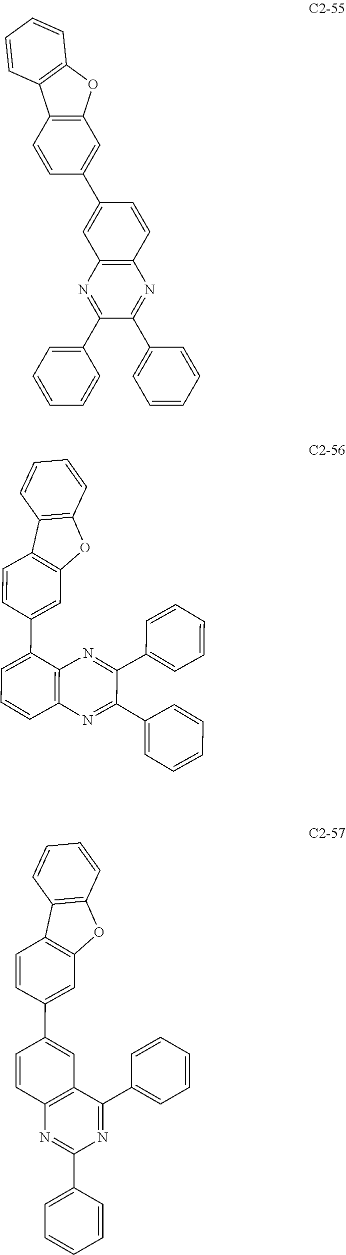 [SMILES: c1ccc(-c2nc3ccc(-c4ccc5c(c4)oc4ccccc45)cc3nc2-c2ccccc2)cc1 ; c1ccc(-c2nc3cccc(-c4ccc5c(c4)oc4ccccc45)c3nc2-c2ccccc2)cc1 ; c1ccc(-c2nc(-c3ccccc3)c3cc(-c4ccc5c(c4)oc4ccccc45)ccc3n2)cc1]

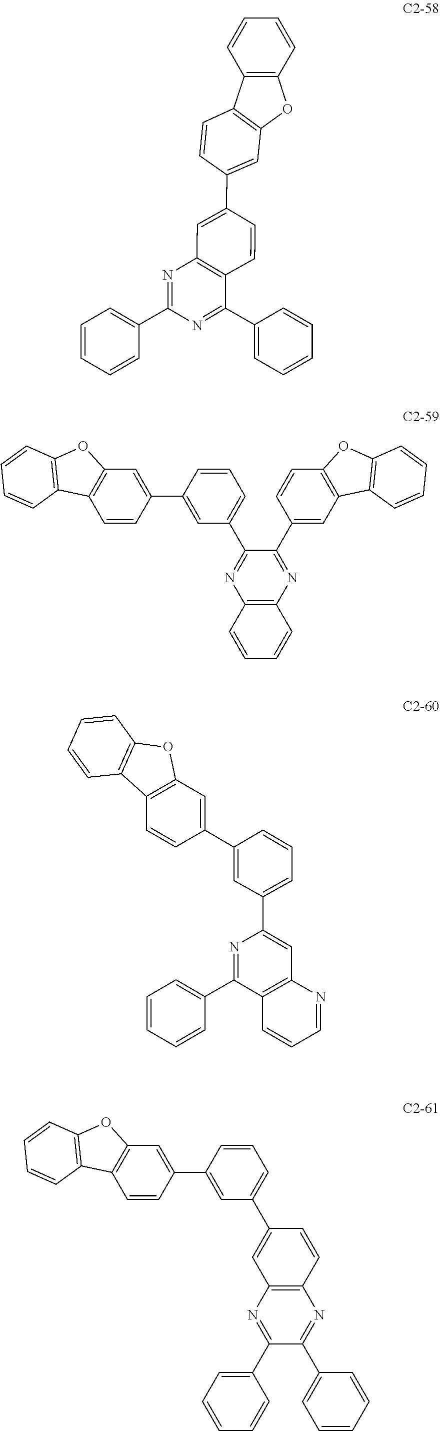 [SMILES: c1ccc(-c2nc(-c3ccccc3)c3ccc(-c4ccc5c(c4)oc4ccccc45)cc3n2)cc1 ; c1cc(-c2ccc3c(c2)oc2ccccc23)cc(-c2nc3ccccc3nc2-c2ccc3oc4ccccc4c3c2)c1 ; c1ccc(-c2nc(-c3cccc(-c4ccc5c(c4)oc4ccccc45)c3)cc3ncccc23)cc1 ; c1ccc(-c2nc3ccc(-c4cccc(-c5ccc6c(c5)oc5ccccc56)c4)cc3nc2-c2ccccc2)cc1]

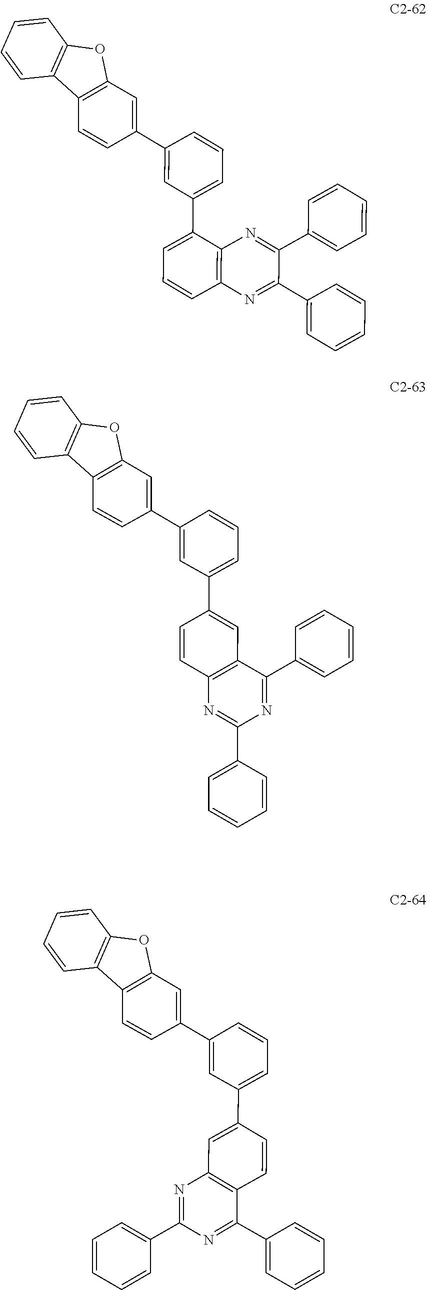 [SMILES: c1ccc(-c2nc3cccc(-c4cccc(-c5ccc6c(c5)oc5ccccc56)c4)c3nc2-c2ccccc2)cc1 ; c1ccc(-c2nc(-c3ccccc3)c3cc(-c4cccc(-c5ccc6c(c5)oc5ccccc56)c4)ccc3n2)cc1 ; c1ccc(-c2nc(-c3ccccc3)c3ccc(-c4cccc(-c5ccc6c(c5)oc5ccccc56)c4)cc3n2)cc1]

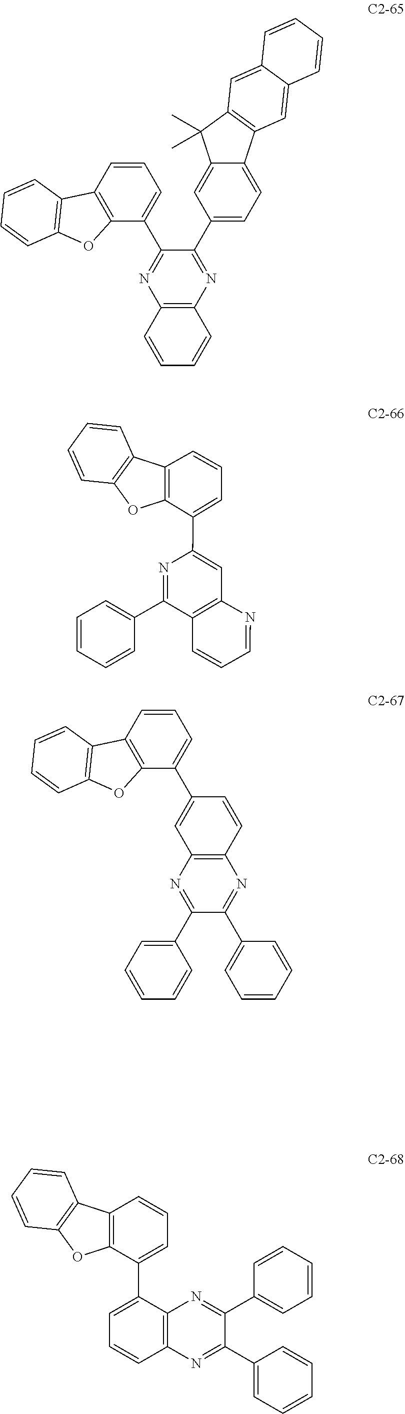 [SMILES: CC1(C)c2cc(-c3nc4ccccc4nc3-c3cccc4c3oc3ccccc34)ccc2-c2cc3ccccc3cc21 ; c1ccc(-c2nc(-c3cccc4c3oc3ccccc34)cc3ncccc23)cc1 ; c1ccc(-c2nc3ccc(-c4cccc5c4oc4ccccc45)cc3nc2-c2ccccc2)cc1 ; c1ccc(-c2nc3cccc(-c4cccc5c4oc4ccccc45)c3nc2-c2ccccc2)cc1]

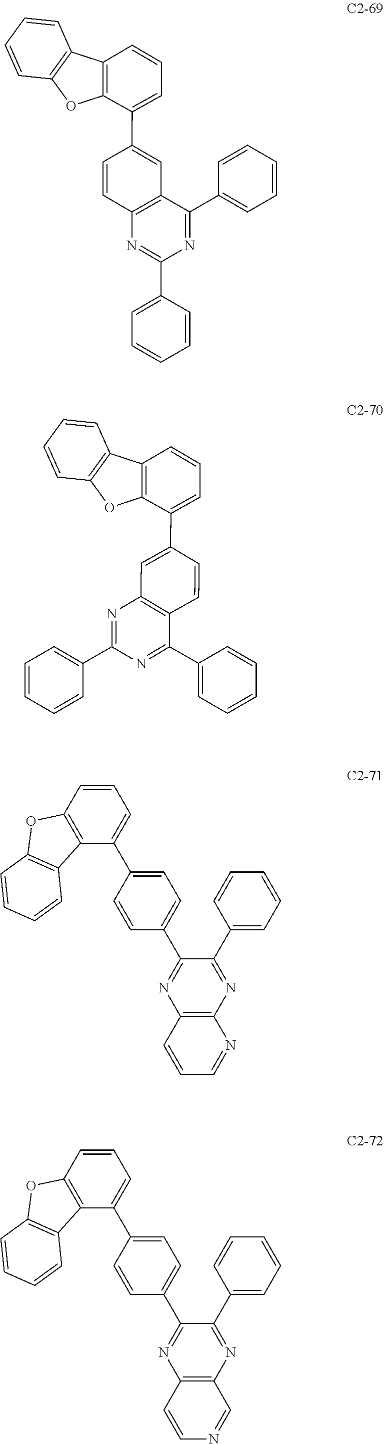 [SMILES: c1ccc(-c2nc(-c3ccccc3)c3cc(-c4cccc5c4oc4ccccc45)ccc3n2)cc1 ; c1ccc(-c2nc(-c3ccccc3)c3ccc(-c4cccc5c4oc4ccccc45)cc3n2)cc1 ; c1ccc(-c2nc3ncccc3nc2-c2ccc(-c3cccc4oc5ccccc5c34)cc2)cc1 ; c1ccc(-c2nc3cnccc3nc2-c2ccc(-c3cccc4oc5ccccc5c34)cc2)cc1]

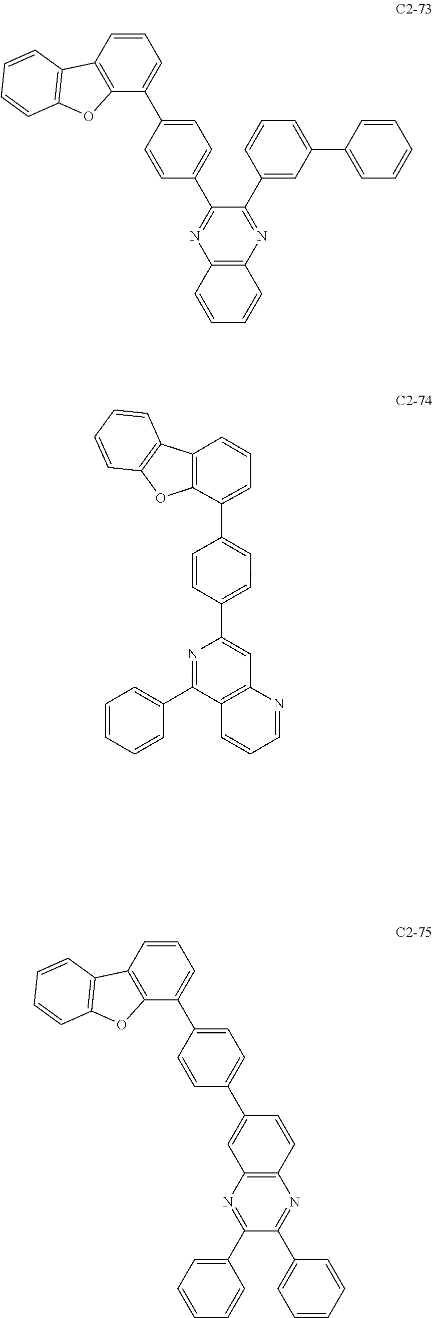 [SMILES: c1ccc(-c2cccc(-c3nc4ccccc4nc3-c3ccc(-c4cccc5c4oc4ccccc45)cc3)c2)cc1 ; c1ccc(-c2nc(-c3ccc(-c4cccc5c4oc4ccccc45)cc3)cc3ncccc23)cc1 ; c1ccc(-c2nc3ccc(-c4ccc(-c5cccc6c5oc5ccccc56)cc4)cc3nc2-c2ccccc2)cc1]

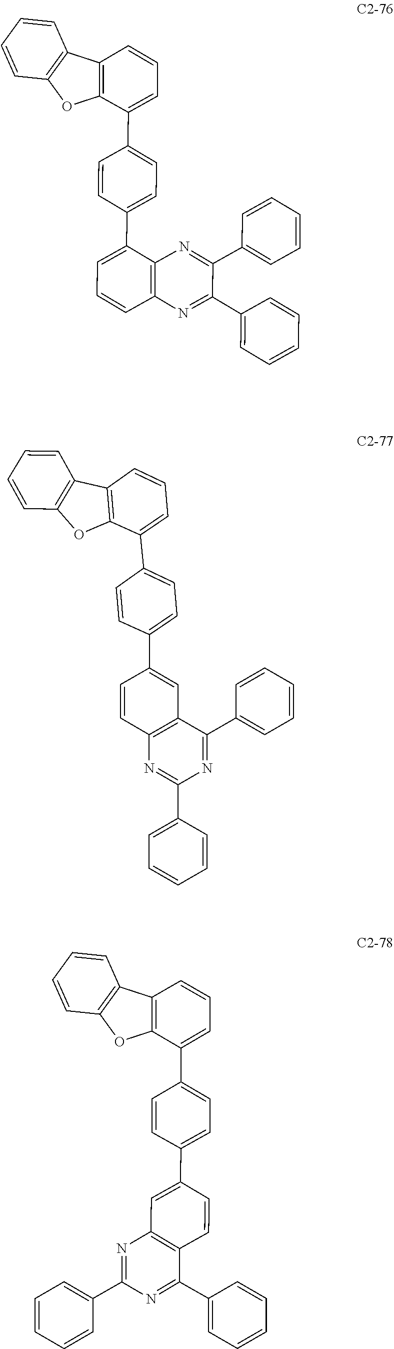 [SMILES: c1ccc(-c2nc3cccc(-c4ccc(-c5cccc6c5oc5ccccc56)cc4)c3nc2-c2ccccc2)cc1 ; c1ccc(-c2nc(-c3ccccc3)c3cc(-c4ccc(-c5cccc6c5oc5ccccc56)cc4)ccc3n2)cc1 ; c1ccc(-c2nc(-c3ccccc3)c3ccc(-c4ccc(-c5cccc6c5oc5ccccc56)cc4)cc3n2)cc1]

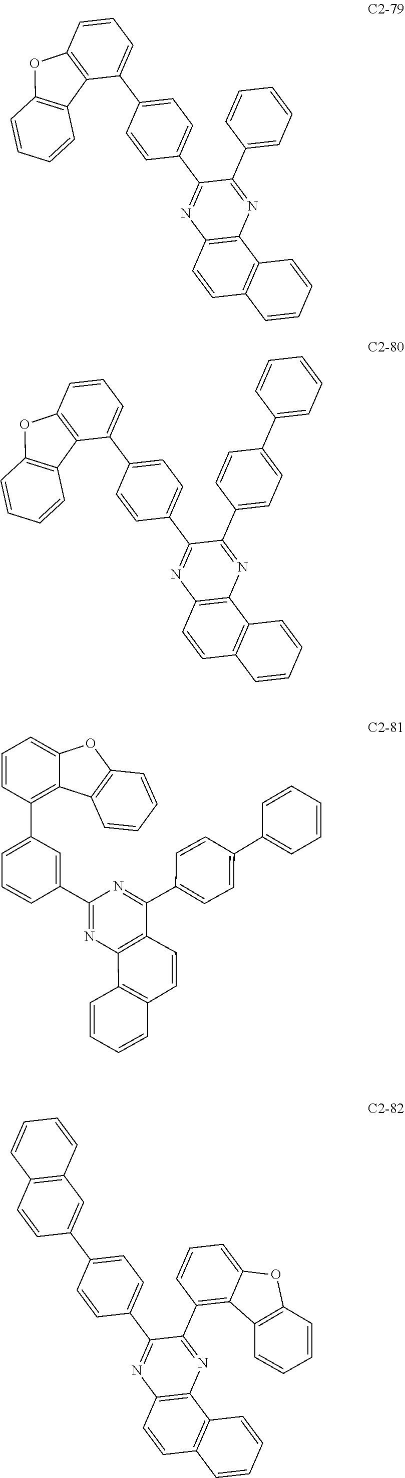

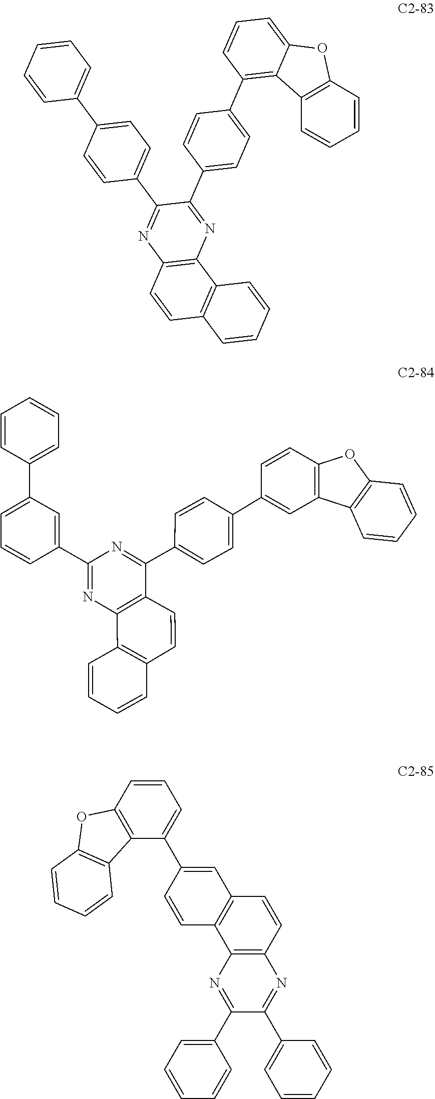 [SMILES: c1ccc(-c2ccc(-c3nc4ccc5ccccc5c4nc3-c3ccc(-c4cccc5oc6ccccc6c45)cc3)cc2)cc1 ; c1ccc(-c2cccc(-c3nc(-c4ccc(-c5ccc6oc7ccccc7c6c5)cc4)c4ccc5ccccc5c4n3)c2)cc1 ; c1ccc(-c2nc3ccc4cc(-c5cccc6oc7ccccc7c56)ccc4c3nc2-c2ccccc2)cc1]

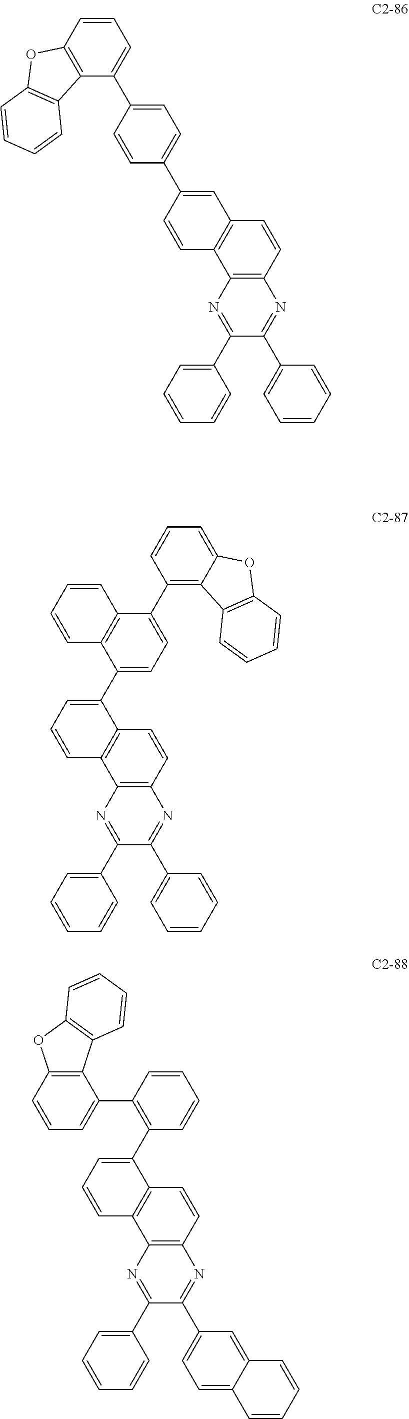

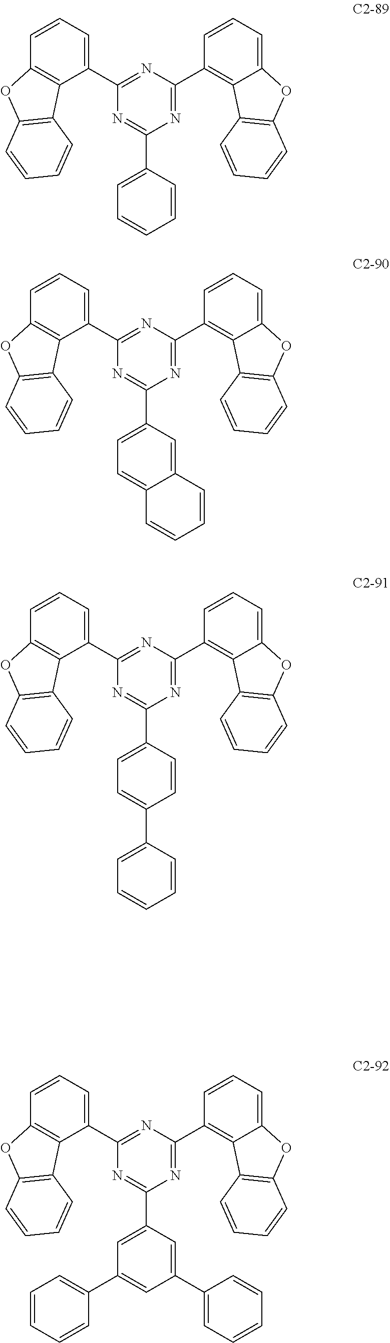 [SMILES: c1ccc(-c2nc(-c3cccc4oc5ccccc5c34)nc(-c3cccc4oc5ccccc5c34)n2)cc1 ; c1ccc2cc(-c3nc(-c4cccc5oc6ccccc6c45)nc(-c4cccc5oc6ccccc6c45)n3)ccc2c1 ; c1ccc(-c2ccc(-c3nc(-c4cccc5oc6ccccc6c45)nc(-c4cccc5oc6ccccc6c45)n3)cc2)cc1 ; c1ccc(-c2cc(-c3ccccc3)cc(-c3nc(-c4cccc5oc6ccccc6c45)nc(-c4cccc5oc6ccccc6c45)n3)c2)cc1]

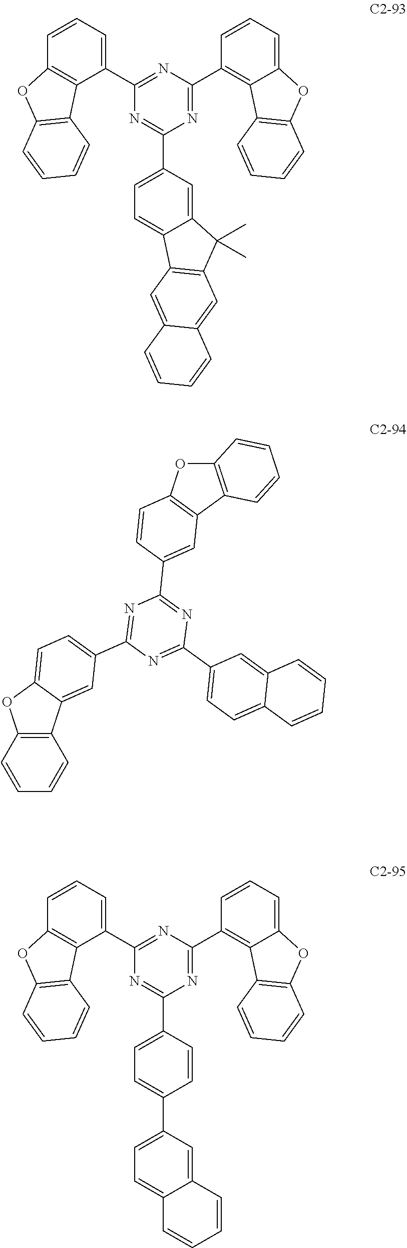 [SMILES: CC1(C)c2cc(-c3nc(-c4cccc5oc6ccccc6c45)nc(-c4cccc5oc6ccccc6c45)n3)ccc2-c2cc3ccccc3cc21 ; c1ccc2cc(-c3nc(-c4ccc5oc6ccccc6c5c4)nc(-c4ccc5oc6ccccc6c5c4)n3)ccc2c1 ; c1ccc2cc(-c3ccc(-c4nc(-c5cccc6oc7ccccc7c56)nc(-c5cccc6oc7ccccc7c56)n4)cc3)ccc2c1]

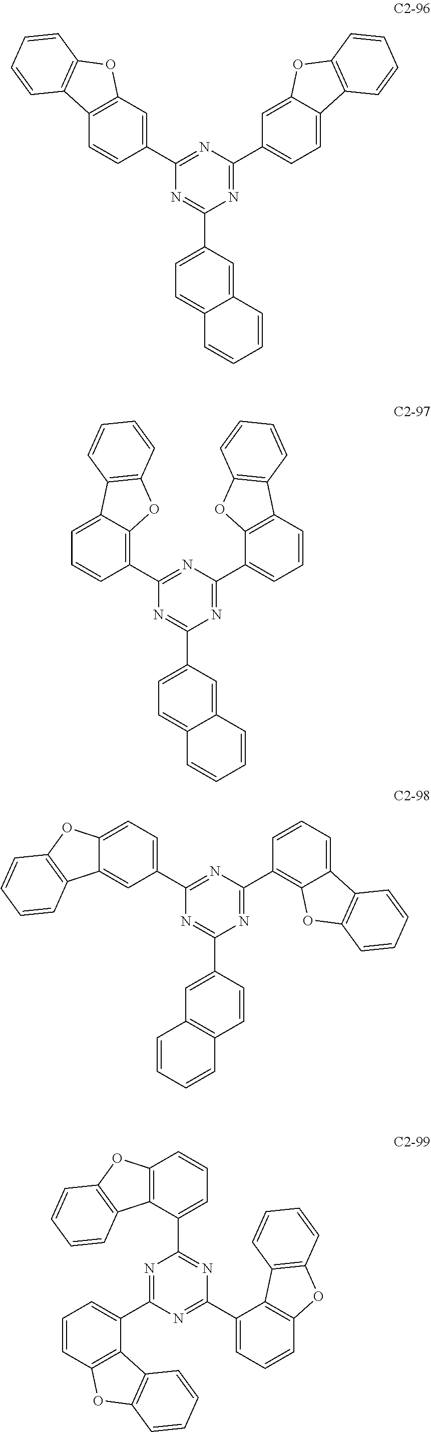 [SMILES: c1ccc2cc(-c3nc(-c4ccc5c(c4)oc4ccccc45)nc(-c4ccc5c(c4)oc4ccccc45)n3)ccc2c1 ; c1ccc2cc(-c3nc(-c4cccc5c4oc4ccccc45)nc(-c4cccc5c4oc4ccccc45)n3)ccc2c1 ; c1ccc2cc(-c3nc(-c4ccc5oc6ccccc6c5c4)nc(-c4cccc5c4oc4ccccc45)n3)ccc2c1 ; c1ccc2c(c1)oc1cccc(-c3nc(-c4cccc5oc6ccccc6c45)nc(-c4cccc5oc6ccccc6c45)n3)c12]

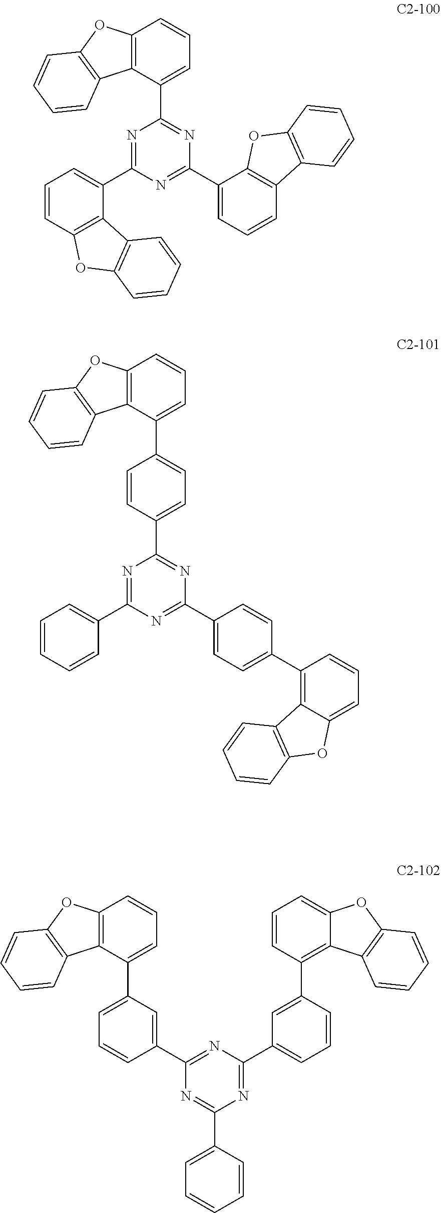 [SMILES: c1ccc2c(c1)oc1c(-c3nc(-c4cccc5oc6ccccc6c45)nc(-c4cccc5oc6ccccc6c45)n3)cccc12 ; c1ccc(-c2nc(-c3ccc(-c4cccc5oc6ccccc6c45)cc3)nc(-c3ccc(-c4cccc5oc6ccccc6c45)cc3)n2)cc1 ; c1ccc(-c2nc(-c3cccc(-c4cccc5oc6ccccc6c45)c3)nc(-c3cccc(-c4cccc5oc6ccccc6c45)c3)n2)cc1]

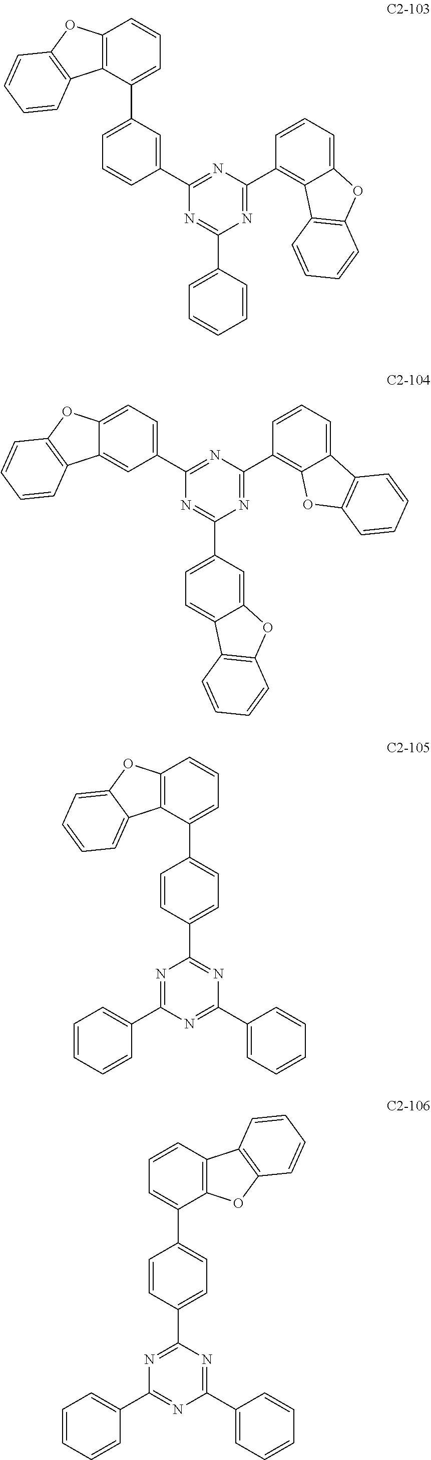

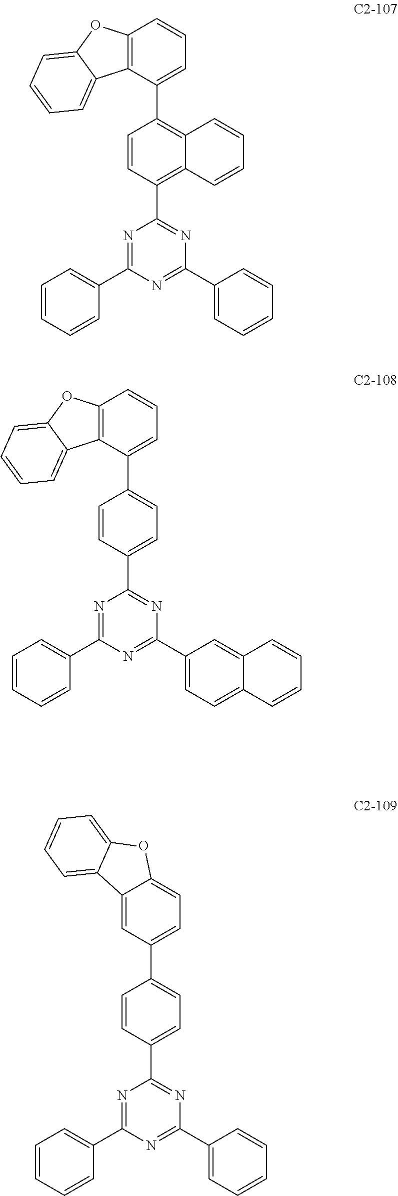 [SMILES: c1ccc(-c2nc(-c3ccccc3)nc(-c3ccc(-c4cccc5oc6ccccc6c45)c4ccccc34)n2)cc1 ; c1ccc(-c2nc(-c3ccc(-c4cccc5oc6ccccc6c45)cc3)nc(-c3ccc4ccccc4c3)n2)cc1 ; c1ccc(-c2nc(-c3ccccc3)nc(-c3ccc(-c4ccc5oc6ccccc6c5c4)cc3)n2)cc1]

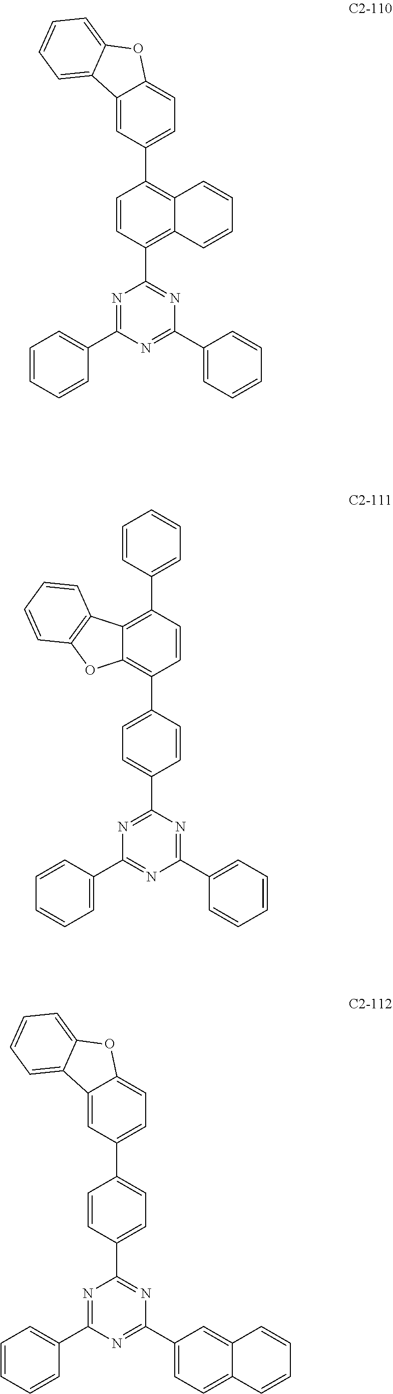 [SMILES: c1ccc(-c2nc(-c3ccccc3)nc(-c3ccc(-c4ccc5oc6ccccc6c5c4)c4ccccc34)n2)cc1 ; c1ccc(-c2nc(-c3ccccc3)nc(-c3ccc(-c4ccc(-c5ccccc5)c5c4oc4ccccc45)cc3)n2)cc1 ; c1ccc(-c2nc(-c3ccc(-c4ccc5oc6ccccc6c5c4)cc3)nc(-c3ccc4ccccc4c3)n2)cc1]

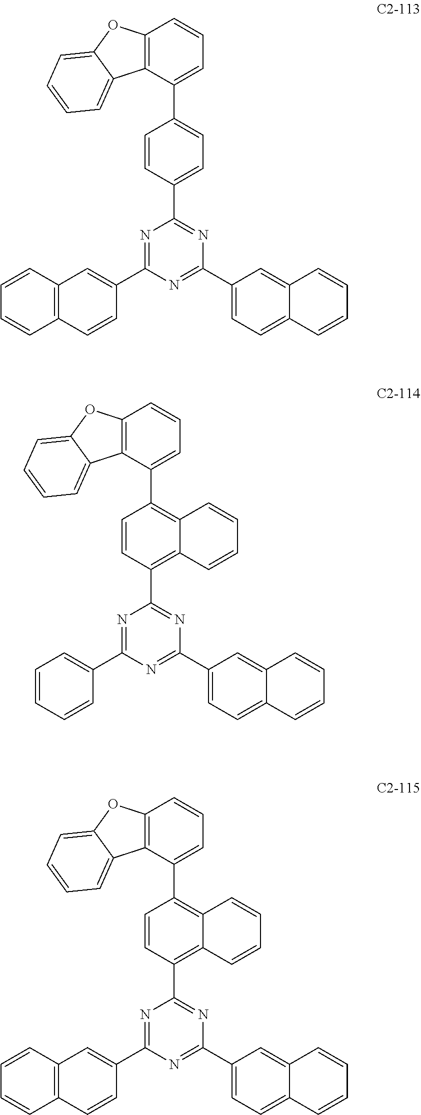 [SMILES: c1ccc2cc(-c3nc(-c4ccc(-c5cccc6oc7ccccc7c56)cc4)nc(-c4ccc5ccccc5c4)n3)ccc2c1 ; c1ccc(-c2nc(-c3ccc4ccccc4c3)nc(-c3ccc(-c4cccc5oc6ccccc6c45)c4ccccc34)n2)cc1 ; c1ccc2cc(-c3nc(-c4ccc5ccccc5c4)nc(-c4ccc(-c5cccc6oc7ccccc7c56)c5ccccc45)n3)ccc2c1]

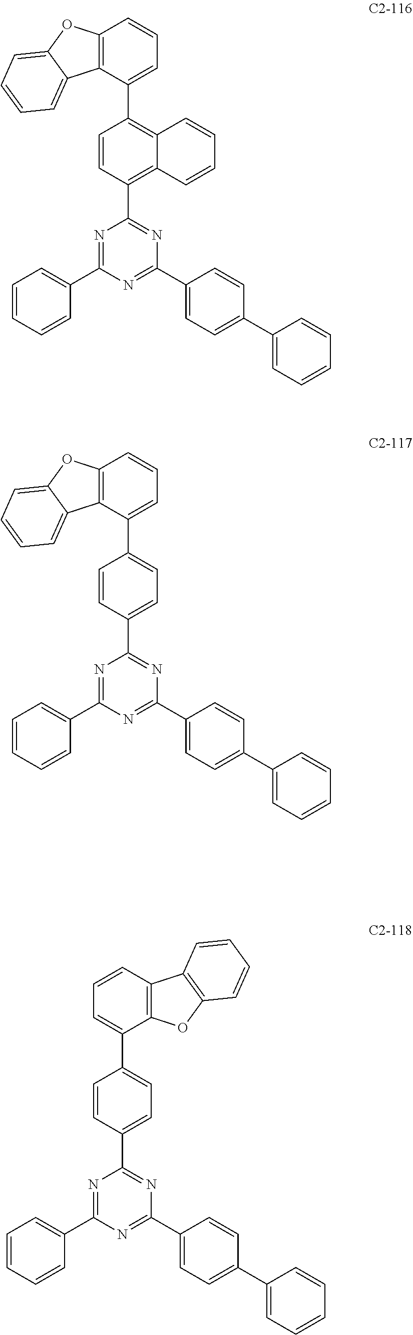 [SMILES: c1ccc(-c2ccc(-c3nc(-c4ccccc4)nc(-c4ccc(-c5cccc6oc7ccccc7c56)c5ccccc45)n3)cc2)cc1 ; c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccccc4)nc(-c4ccc(-c5cccc6c5oc5ccccc56)cc4)n3)cc2)cc1]

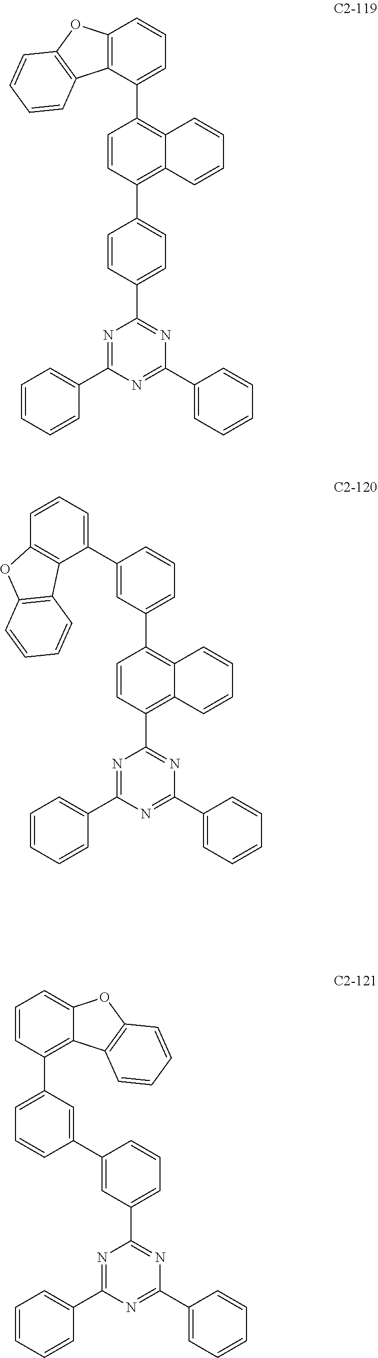 [SMILES: c1ccc(-c2nc(-c3ccccc3)nc(-c3ccc(-c4ccc(-c5cccc6oc7ccccc7c56)c5ccccc45)cc3)n2)cc1 ; c1ccc(-c2nc(-c3ccccc3)nc(-c3ccc(-c4cccc(-c5cccc6oc7ccccc7c56)c4)c4ccccc34)n2)cc1 ; c1ccc(-c2nc(-c3ccccc3)nc(-c3cccc(-c4cccc(-c5cccc6oc7ccccc7c56)c4)c3)n2)cc1]

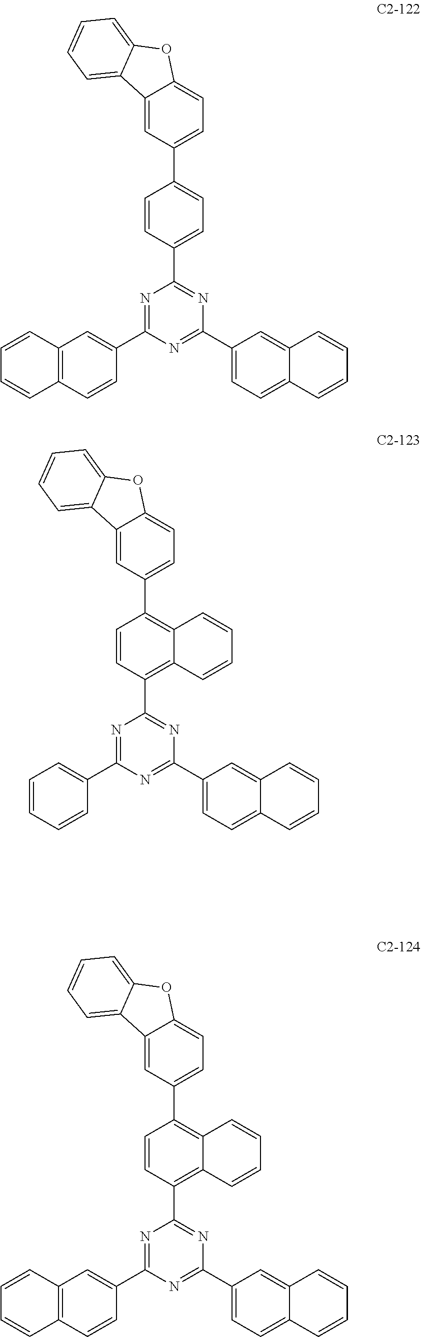 [SMILES: c1ccc2cc(-c3nc(-c4ccc(-c5ccc6oc7ccccc7c6c5)cc4)nc(-c4ccc5ccccc5c4)n3)ccc2c1 ; c1ccc(-c2nc(-c3ccc4ccccc4c3)nc(-c3ccc(-c4ccc5oc6ccccc6c5c4)c4ccccc34)n2)cc1 ; c1ccc2cc(-c3nc(-c4ccc5ccccc5c4)nc(-c4ccc(-c5ccc6oc7ccccc7c6c5)c5ccccc45)n3)ccc2c1]

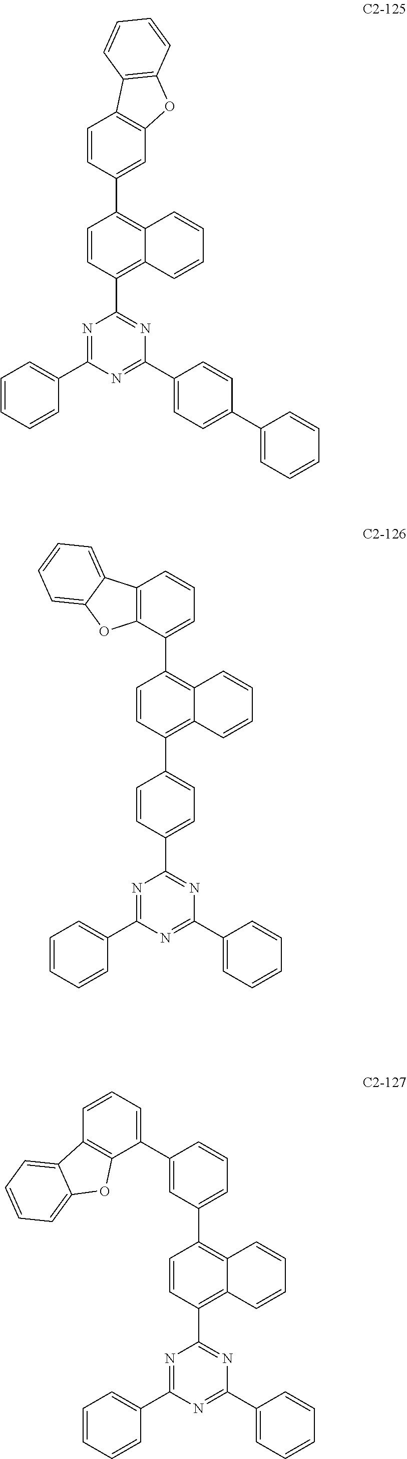 [SMILES: c1ccc(-c2ccc(-c3nc(-c4ccccc4)nc(-c4ccc(-c5ccc6c(c5)oc5ccccc56)c5ccccc45)n3)cc2)cc1 ; c1ccc(-c2nc(-c3ccccc3)nc(-c3ccc(-c4ccc(-c5cccc6c5oc5ccccc56)c5ccccc45)cc3)n2)cc1 ; c1ccc(-c2nc(-c3ccccc3)nc(-c3ccc(-c4cccc(-c5cccc6c5oc5ccccc56)c4)c4ccccc34)n2)cc1]

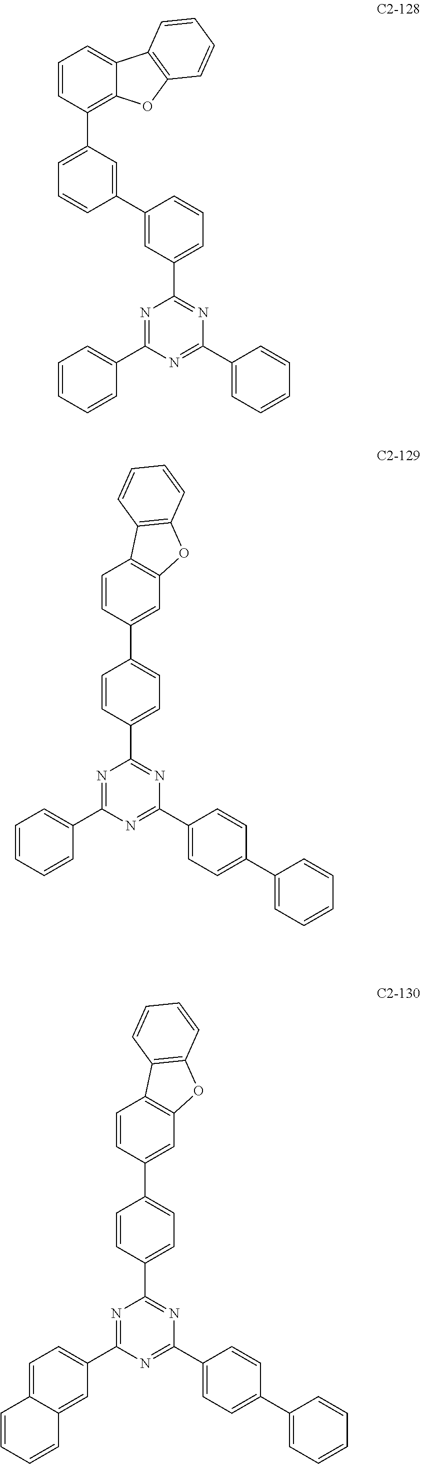 [SMILES: c1ccc(-c2nc(-c3ccccc3)nc(-c3cccc(-c4cccc(-c5cccc6c5oc5ccccc56)c4)c3)n2)cc1 ; c1ccc(-c2ccc(-c3nc(-c4ccccc4)nc(-c4ccc(-c5ccc6c(c5)oc5ccccc56)cc4)n3)cc2)cc1 ; c1ccc(-c2ccc(-c3nc(-c4ccc(-c5ccc6c(c5)oc5ccccc56)cc4)nc(-c4ccc5ccccc5c4)n3)cc2)cc1]

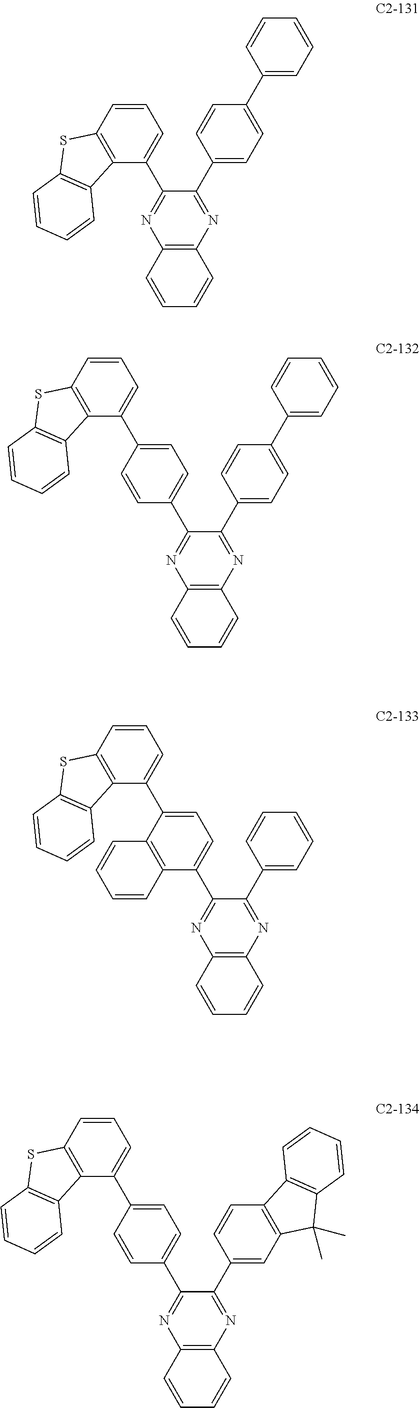 [SMILES: c1ccc(-c2ccc(-c3nc4ccccc4nc3-c3cccc4sc5ccccc5c34)cc2)cc1 ; c1ccc(-c2ccc(-c3nc4ccccc4nc3-c3ccc(-c4cccc5sc6ccccc6c45)cc3)cc2)cc1 ; c1ccc(-c2nc3ccccc3nc2-c2ccc(-c3cccc4sc5ccccc5c34)c3ccccc23)cc1 ; CC1(C)c2ccccc2-c2ccc(-c3nc4ccccc4nc3-c3ccc(-c4cccc5sc6ccccc6c45)cc3)cc21]

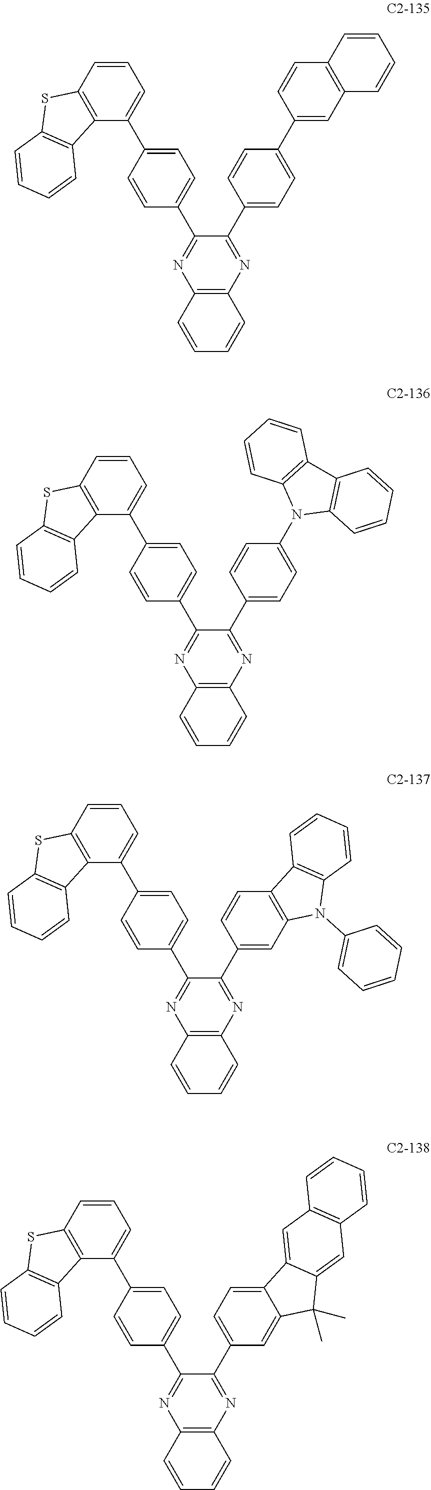 [SMILES: c1ccc2cc(-c3ccc(-c4nc5ccccc5nc4-c4ccc(-c5cccc6sc7ccccc7c56)cc4)cc3)ccc2c1 ; c1ccc2nc(-c3ccc(-n4c5ccccc5c5ccccc54)cc3)c(-c3ccc(-c4cccc5sc6ccccc6c45)cc3)nc2c1 ; c1ccc(-n2c3ccccc3c3ccc(-c4nc5ccccc5nc4-c4ccc(-c5cccc6sc7ccccc7c56)cc4)cc32)cc1 ; CC1(C)c2cc(-c3nc4ccccc4nc3-c3ccc(-c4cccc5sc6ccccc6c45)cc3)ccc2-c2cc3ccccc3cc21]

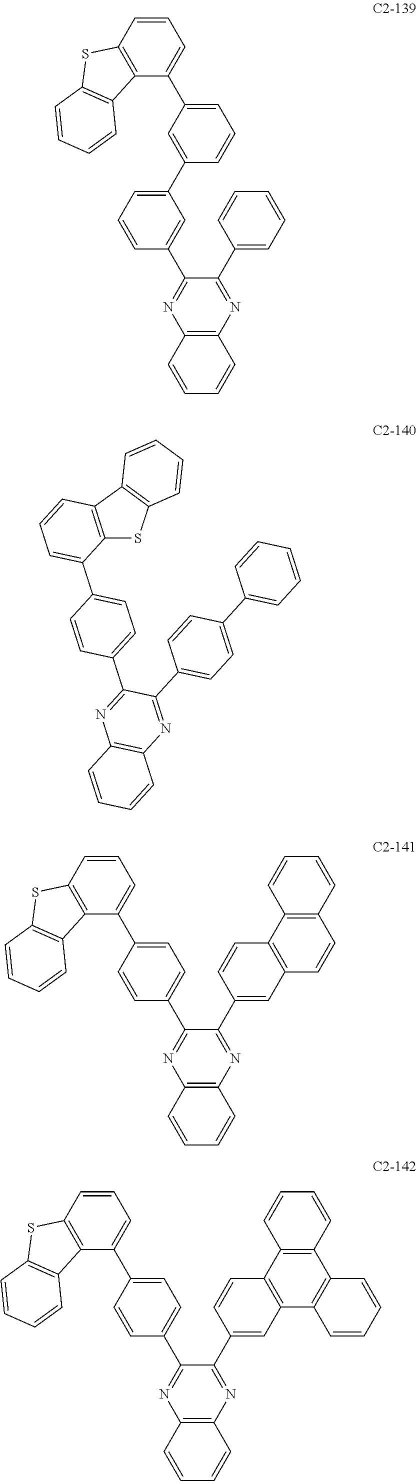 [SMILES: c1ccc(-c2nc3ccccc3nc2-c2cccc(-c3cccc(-c4cccc5sc6ccccc6c45)c3)c2)cc1 ; c1ccc(-c2ccc(-c3nc4ccccc4nc3-c3ccc(-c4cccc5c4sc4ccccc45)cc3)cc2)cc1 ; c1ccc2c(c1)ccc1cc(-c3nc4ccccc4nc3-c3ccc(-c4cccc5sc6ccccc6c45)cc3)ccc12 ; c1ccc2nc(-c3ccc4c5ccccc5c5ccccc5c4c3)c(-c3ccc(-c4cccc5sc6ccccc6c45)cc3)nc2c1]

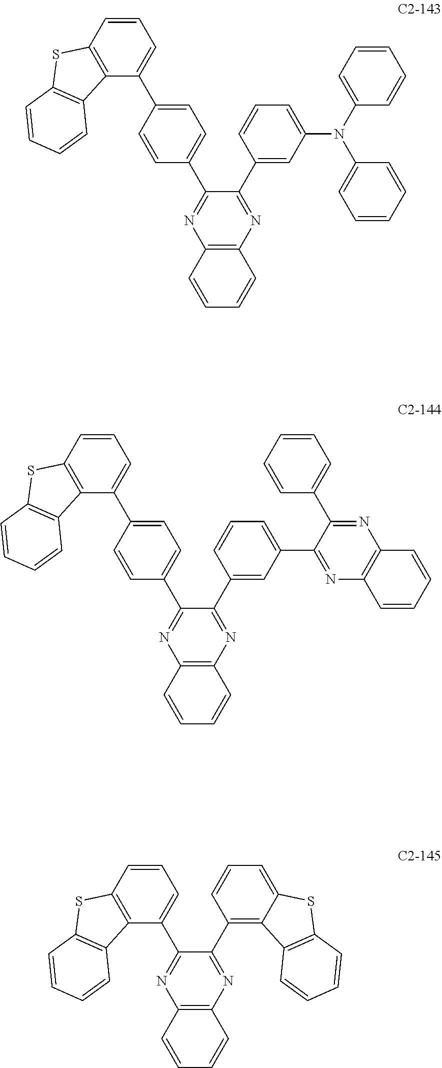 [SMILES: c1ccc(N(c2ccccc2)c2cccc(-c3nc4ccccc4nc3-c3ccc(-c4cccc5sc6ccccc6c45)cc3)c2)cc1 ; c1ccc(-c2nc3ccccc3nc2-c2cccc(-c3nc4ccccc4nc3-c3ccc(-c4cccc5sc6ccccc6c45)cc3)c2)cc1 ; c1ccc2nc(-c3cccc4sc5ccccc5c34)c(-c3cccc4sc5ccccc5c34)nc2c1]

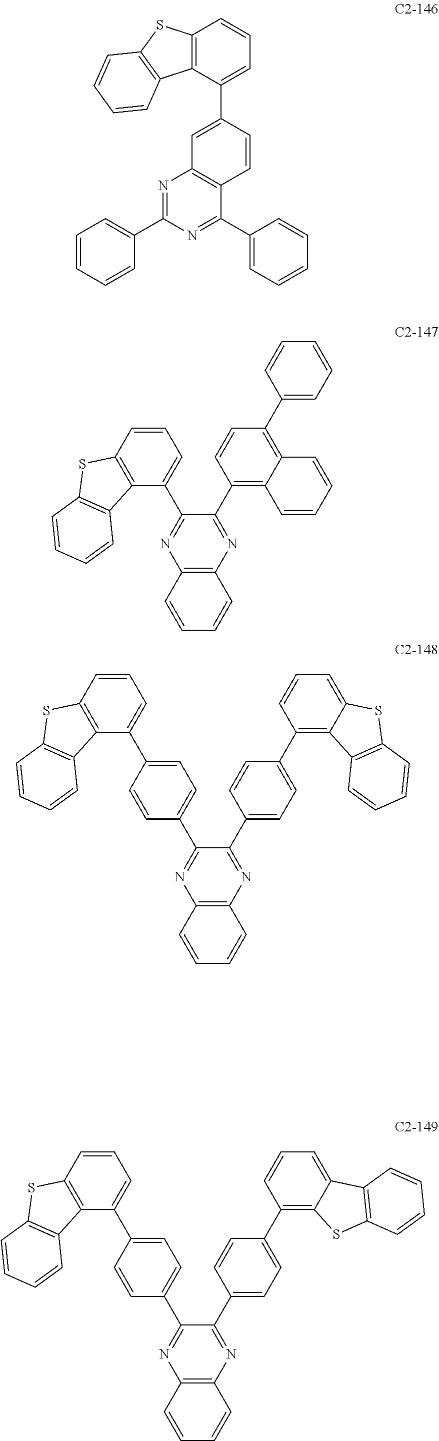 [SMILES: c1ccc(-c2nc(-c3ccccc3)c3ccc(-c4cccc5sc6ccccc6c45)cc3n2)cc1 ; c1ccc(-c2ccc(-c3nc4ccccc4nc3-c3cccc4sc5ccccc5c34)c3ccccc23)cc1 ; c1ccc2nc(-c3ccc(-c4cccc5sc6ccccc6c45)cc3)c(-c3ccc(-c4cccc5sc6ccccc6c45)cc3)nc2c1 ; c1ccc2nc(-c3ccc(-c4cccc5sc6ccccc6c45)cc3)c(-c3ccc(-c4cccc5c4sc4ccccc45)cc3)nc2c1]

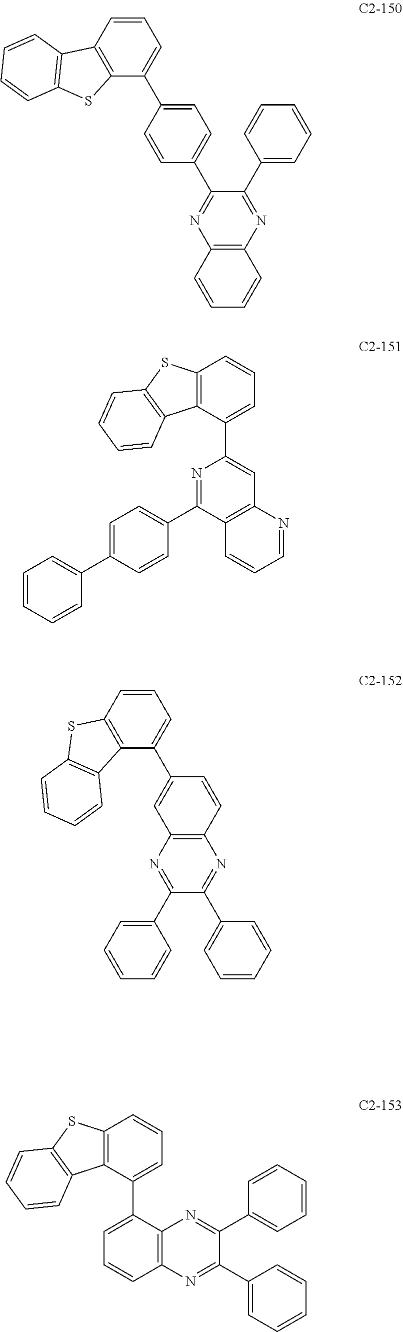 [SMILES: c1ccc(-c2nc3ccccc3nc2-c2ccc(-c3cccc4c3sc3ccccc34)cc2)cc1 ; c1ccc(-c2ccc(-c3nc(-c4cccc5sc6ccccc6c45)cc4ncccc34)cc2)cc1 ; c1ccc(-c2nc3ccc(-c4cccc5sc6ccccc6c45)cc3nc2-c2ccccc2)cc1 ; c1ccc(-c2nc3cccc(-c4cccc5sc6ccccc6c45)c3nc2-c2ccccc2)cc1]

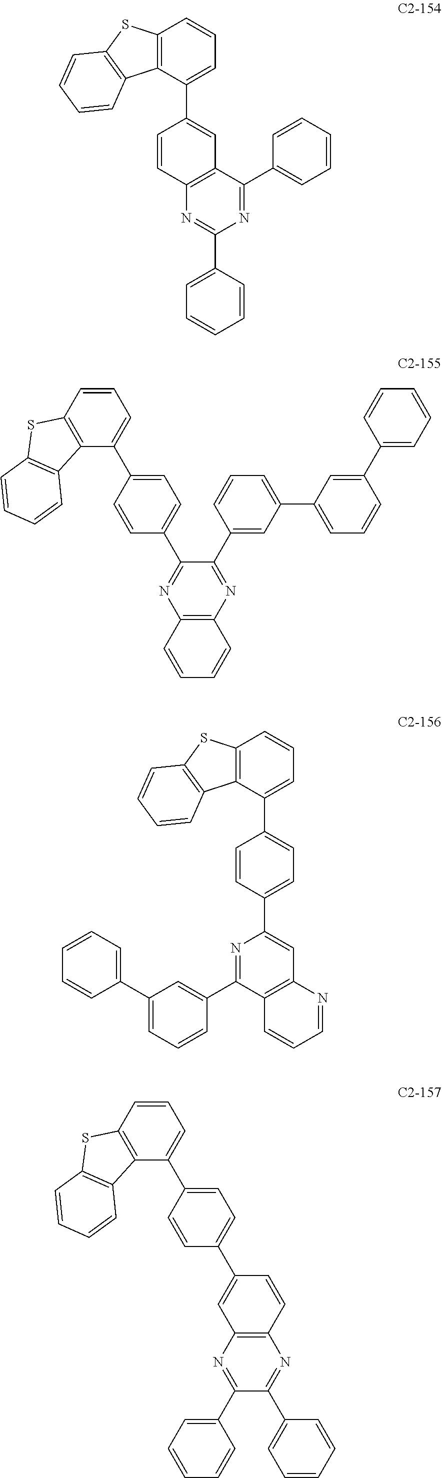 [SMILES: c1ccc(-c2nc(-c3ccccc3)c3cc(-c4cccc5sc6ccccc6c45)ccc3n2)cc1 ; c1ccc(-c2cccc(-c3cccc(-c4nc5ccccc5nc4-c4ccc(-c5cccc6sc7ccccc7c56)cc4)c3)c2)cc1 ; c1ccc(-c2cccc(-c3nc(-c4ccc(-c5cccc6sc7ccccc7c56)cc4)cc4ncccc34)c2)cc1 ; c1ccc(-c2nc3ccc(-c4ccc(-c5cccc6sc7ccccc7c56)cc4)cc3nc2-c2ccccc2)cc1]

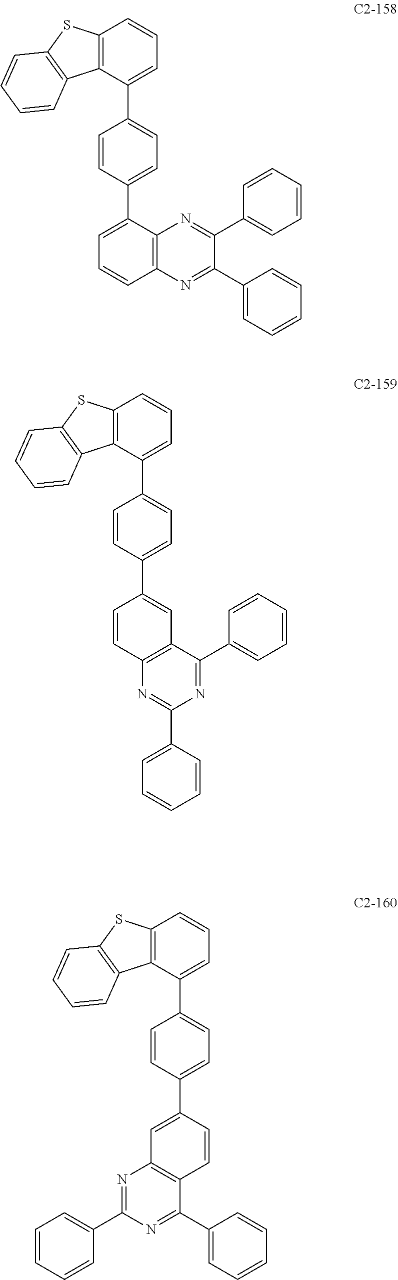 [SMILES: c1ccc(-c2nc3cccc(-c4ccc(-c5cccc6sc7ccccc7c56)cc4)c3nc2-c2ccccc2)cc1 ; c1ccc(-c2nc(-c3ccccc3)c3cc(-c4ccc(-c5cccc6sc7ccccc7c56)cc4)ccc3n2)cc1 ; c1ccc(-c2nc(-c3ccccc3)c3ccc(-c4ccc(-c5cccc6sc7ccccc7c56)cc4)cc3n2)cc1]

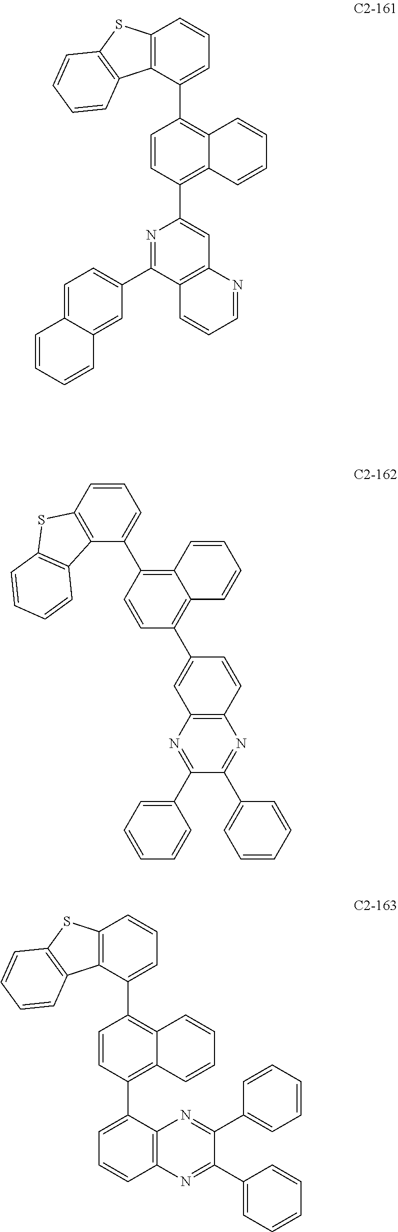 [SMILES: c1ccc2cc(-c3nc(-c4ccc(-c5cccc6sc7ccccc7c56)c5ccccc45)cc4ncccc34)ccc2c1 ; c1ccc(-c2nc3ccc(-c4ccc(-c5cccc6sc7ccccc7c56)c5ccccc45)cc3nc2-c2ccccc2)cc1 ; c1ccc(-c2nc3cccc(-c4ccc(-c5cccc6sc7ccccc7c56)c5ccccc45)c3nc2-c2ccccc2)cc1]

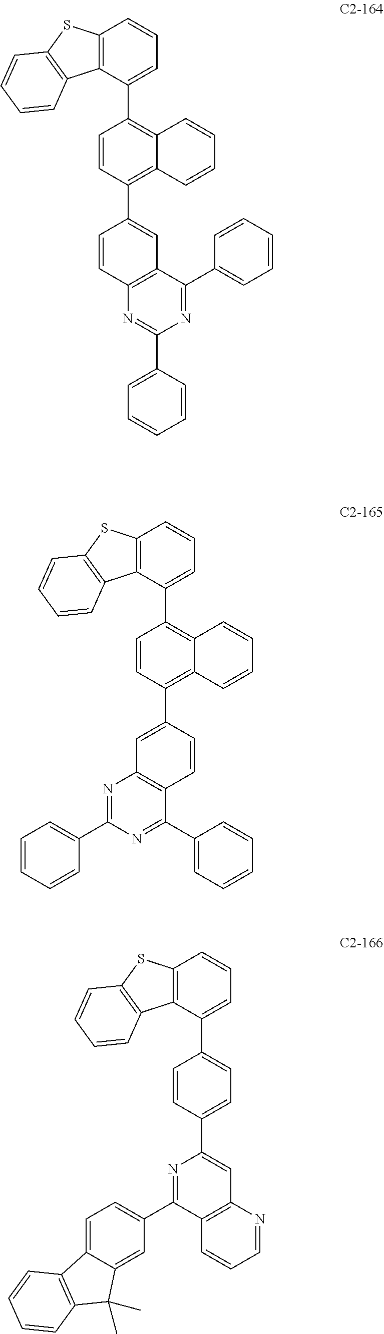 [SMILES: c1ccc(-c2nc(-c3ccccc3)c3cc(-c4ccc(-c5cccc6sc7ccccc7c56)c5ccccc45)ccc3n2)cc1 ; c1ccc(-c2nc(-c3ccccc3)c3ccc(-c4ccc(-c5cccc6sc7ccccc7c56)c5ccccc45)cc3n2)cc1 ; CC1(C)c2ccccc2-c2ccc(-c3nc(-c4ccc(-c5cccc6sc7ccccc7c56)cc4)cc4ncccc34)cc21]

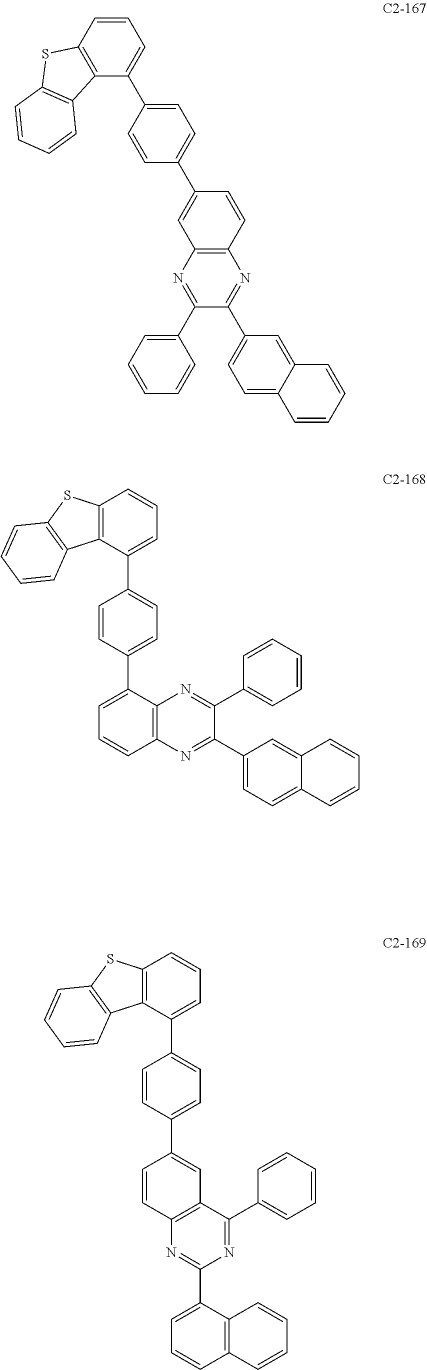 [SMILES: c1ccc(-c2nc3cc(-c4ccc(-c5cccc6sc7ccccc7c56)cc4)ccc3nc2-c2ccc3ccccc3c2)cc1 ; c1ccc(-c2nc3c(-c4ccc(-c5cccc6sc7ccccc7c56)cc4)cccc3nc2-c2ccc3ccccc3c2)cc1 ; c1ccc(-c2nc(-c3cccc4ccccc34)nc3ccc(-c4ccc(-c5cccc6sc7ccccc7c56)cc4)cc23)cc1]

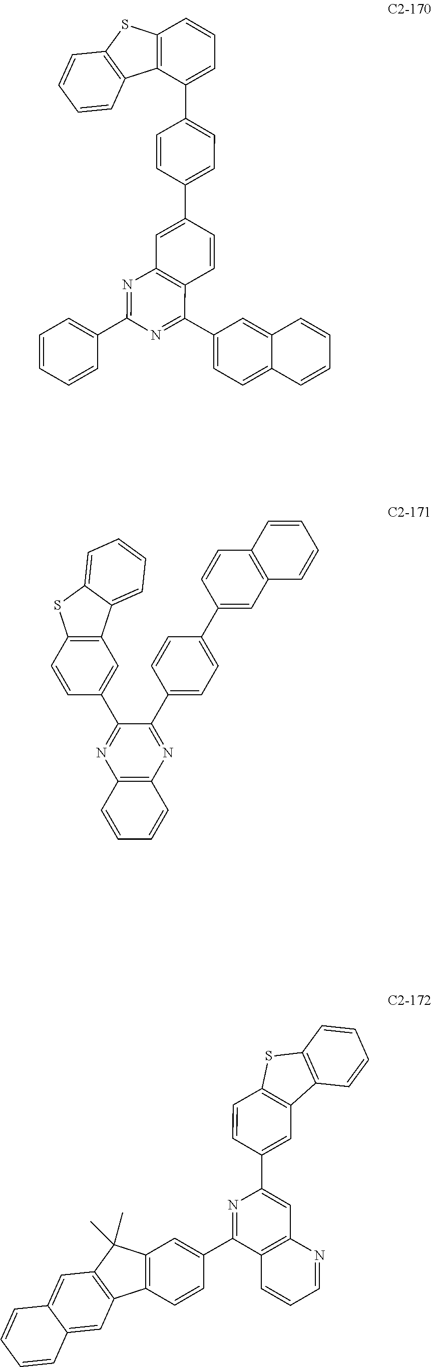 [SMILES: c1ccc(-c2nc(-c3ccc4ccccc4c3)c3ccc(-c4ccc(-c5cccc6sc7ccccc7c56)cc4)cc3n2)cc1 ; c1ccc2cc(-c3ccc(-c4nc5ccccc5nc4-c4ccc5sc6ccccc6c5c4)cc3)ccc2c1 ; CC1(C)c2cc(-c3nc(-c4ccc5sc6ccccc6c5c4)cc4ncccc34)ccc2-c2cc3ccccc3cc21]

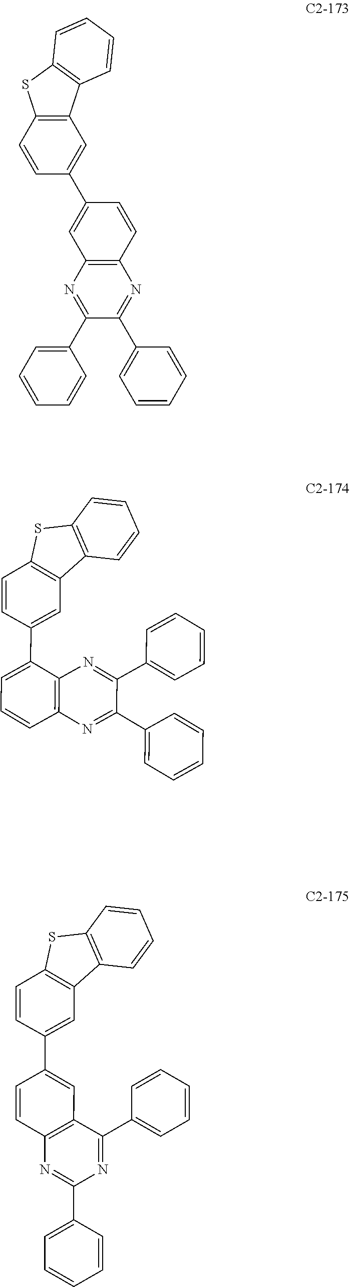 [SMILES: c1ccc(-c2nc3ccc(-c4ccc5sc6ccccc6c5c4)cc3nc2-c2ccccc2)cc1 ; c1ccc(-c2nc3cccc(-c4ccc5sc6ccccc6c5c4)c3nc2-c2ccccc2)cc1 ; c1ccc(-c2nc(-c3ccccc3)c3cc(-c4ccc5sc6ccccc6c5c4)ccc3n2)cc1]

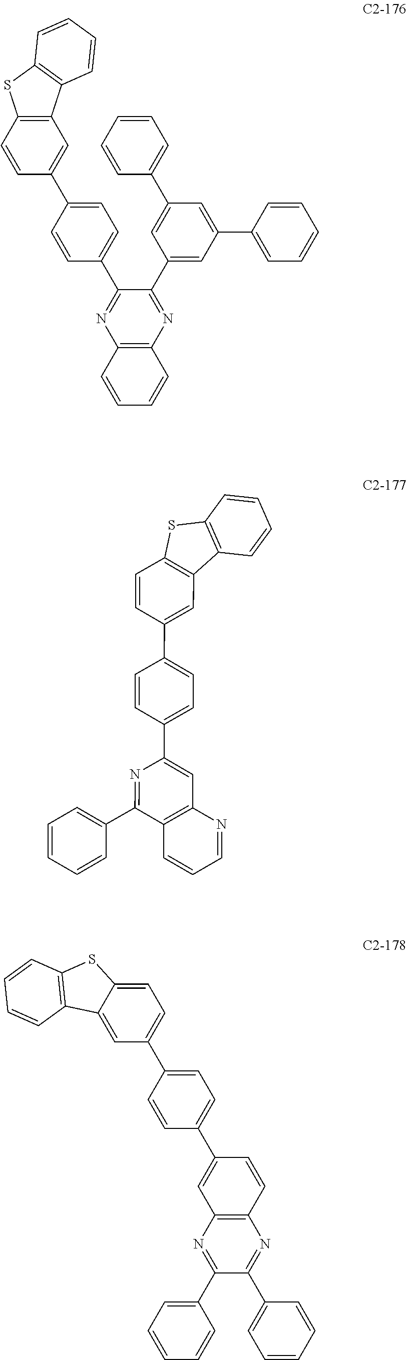 [SMILES: c1ccc(-c2cc(-c3ccccc3)cc(-c3nc4ccccc4nc3-c3ccc(-c4ccc5sc6ccccc6c5c4)cc3)c2)cc1 ; c1ccc(-c2nc(-c3ccc(-c4ccc5sc6ccccc6c5c4)cc3)cc3ncccc23)cc1 ; c1ccc(-c2nc3ccc(-c4ccc(-c5ccc6sc7ccccc7c6c5)cc4)cc3nc2-c2ccccc2)cc1]

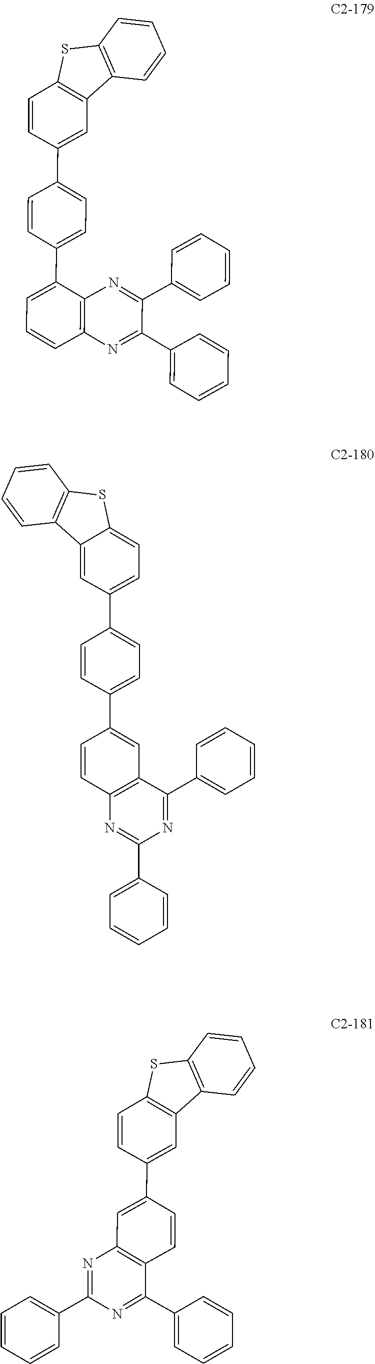 [SMILES: c1ccc(-c2nc3cccc(-c4ccc(-c5ccc6sc7ccccc7c6c5)cc4)c3nc2-c2ccccc2)cc1 ; c1ccc(-c2nc(-c3ccccc3)c3cc(-c4ccc(-c5ccc6sc7ccccc7c6c5)cc4)ccc3n2)cc1 ; c1ccc(-c2nc(-c3ccccc3)c3ccc(-c4ccc5sc6ccccc6c5c4)cc3n2)cc1]

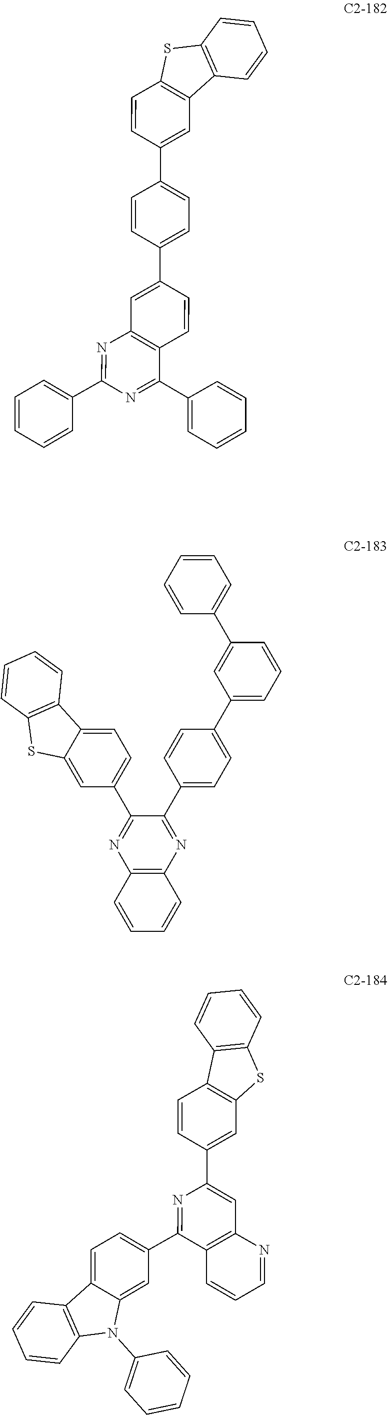 [SMILES: c1ccc(-c2nc(-c3ccccc3)c3ccc(-c4ccc(-c5ccc6sc7ccccc7c6c5)cc4)cc3n2)cc1 ; c1ccc(-c2cccc(-c3ccc(-c4nc5ccccc5nc4-c4ccc5c(c4)sc4ccccc45)cc3)c2)cc1 ; c1ccc(-n2c3ccccc3c3ccc(-c4nc(-c5ccc6c(c5)sc5ccccc56)cc5ncccc45)cc32)cc1]

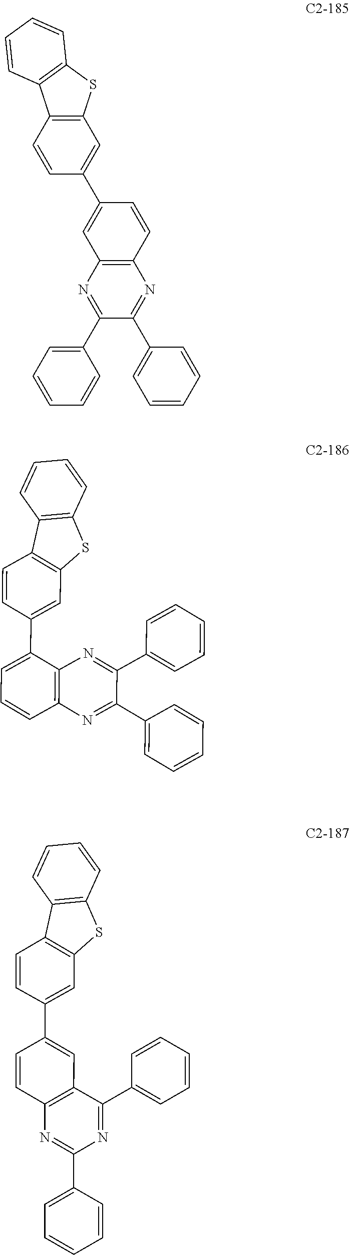 [SMILES: c1ccc(-c2nc3ccc(-c4ccc5c(c4)sc4ccccc45)cc3nc2-c2ccccc2)cc1 ; c1ccc(-c2nc3cccc(-c4ccc5c(c4)sc4ccccc45)c3nc2-c2ccccc2)cc1 ; c1ccc(-c2nc(-c3ccccc3)c3cc(-c4ccc5c(c4)sc4ccccc45)ccc3n2)cc1]

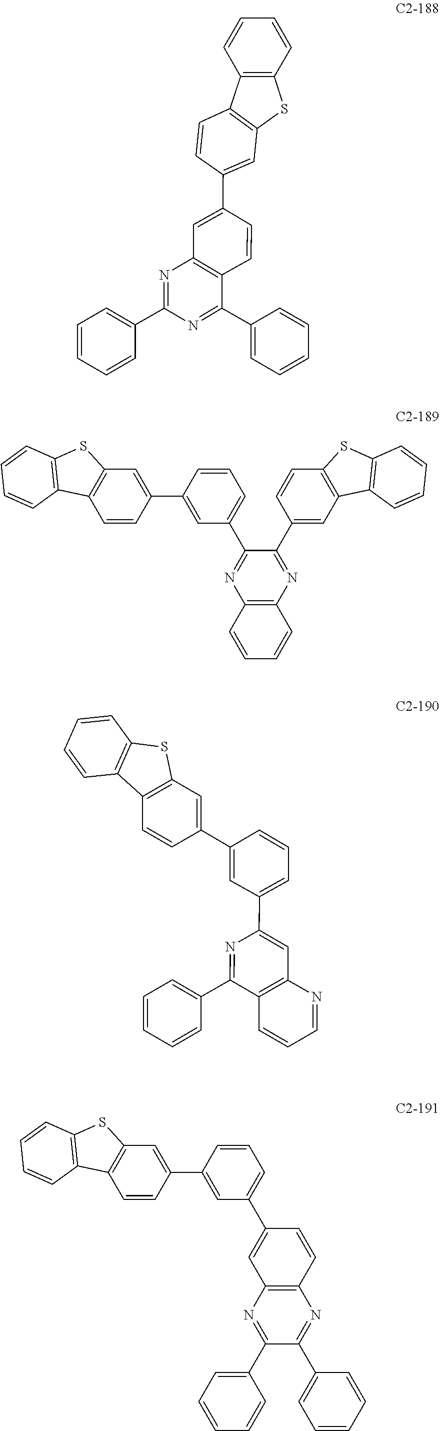 [SMILES: c1ccc(-c2nc(-c3ccccc3)c3ccc(-c4ccc5c(c4)sc4ccccc45)cc3n2)cc1 ; c1cc(-c2ccc3c(c2)sc2ccccc23)cc(-c2nc3ccccc3nc2-c2ccc3sc4ccccc4c3c2)c1 ; c1ccc(-c2nc(-c3cccc(-c4ccc5c(c4)sc4ccccc45)c3)cc3ncccc23)cc1 ; c1ccc(-c2nc3ccc(-c4cccc(-c5ccc6c(c5)sc5ccccc56)c4)cc3nc2-c2ccccc2)cc1]

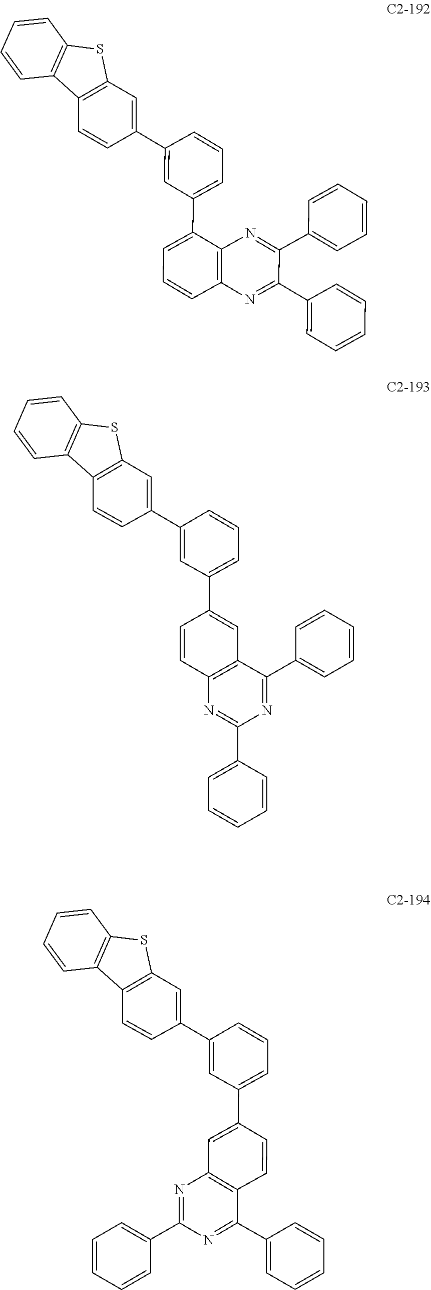 [SMILES: c1ccc(-c2nc3cccc(-c4cccc(-c5ccc6c(c5)sc5ccccc56)c4)c3nc2-c2ccccc2)cc1 ; c1ccc(-c2nc(-c3ccccc3)c3cc(-c4cccc(-c5ccc6c(c5)sc5ccccc56)c4)ccc3n2)cc1 ; c1ccc(-c2nc(-c3ccccc3)c3ccc(-c4cccc(-c5ccc6c(c5)sc5ccccc56)c4)cc3n2)cc1]

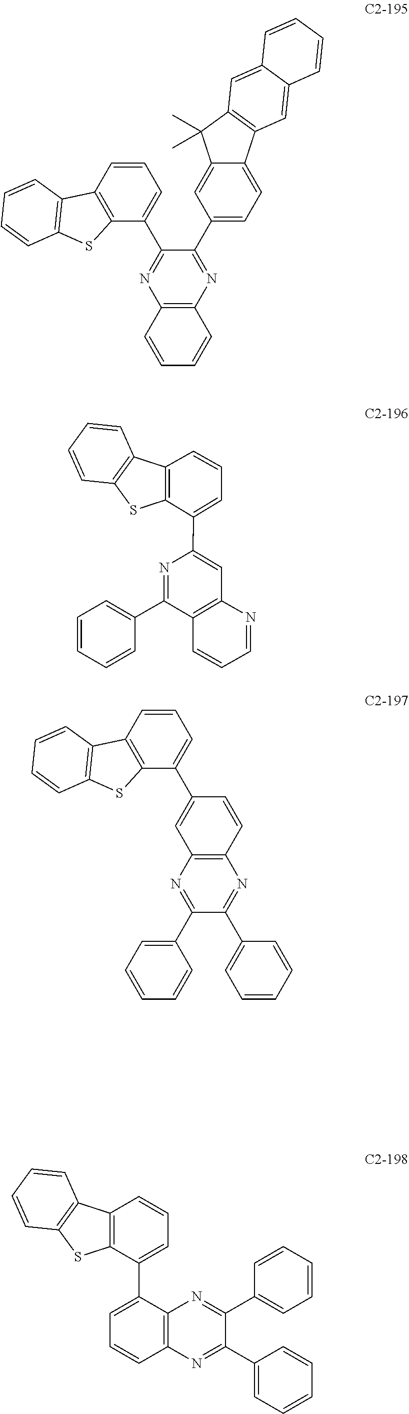 [SMILES: CC1(C)c2cc(-c3nc4ccccc4nc3-c3cccc4c3sc3ccccc34)ccc2-c2cc3ccccc3cc21 ; c1ccc(-c2nc(-c3cccc4c3sc3ccccc34)cc3ncccc23)cc1 ; c1ccc(-c2nc3ccc(-c4cccc5c4sc4ccccc45)cc3nc2-c2ccccc2)cc1 ; c1ccc(-c2nc3cccc(-c4cccc5c4sc4ccccc45)c3nc2-c2ccccc2)cc1]

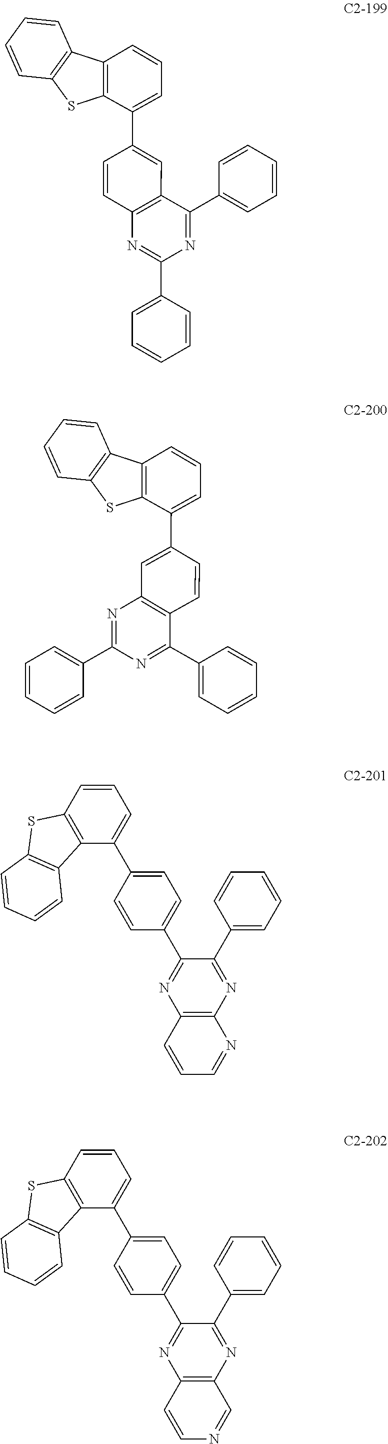 [SMILES: c1ccc(-c2nc(-c3ccccc3)c3cc(-c4cccc5c4sc4ccccc45)ccc3n2)cc1 ; c1ccc(-c2nc(-c3ccccc3)c3ccc(-c4cccc5c4sc4ccccc45)cc3n2)cc1 ; c1ccc(-c2nc3ncccc3nc2-c2ccc(-c3cccc4sc5ccccc5c34)cc2)cc1 ; c1ccc(-c2nc3cnccc3nc2-c2ccc(-c3cccc4sc5ccccc5c34)cc2)cc1]

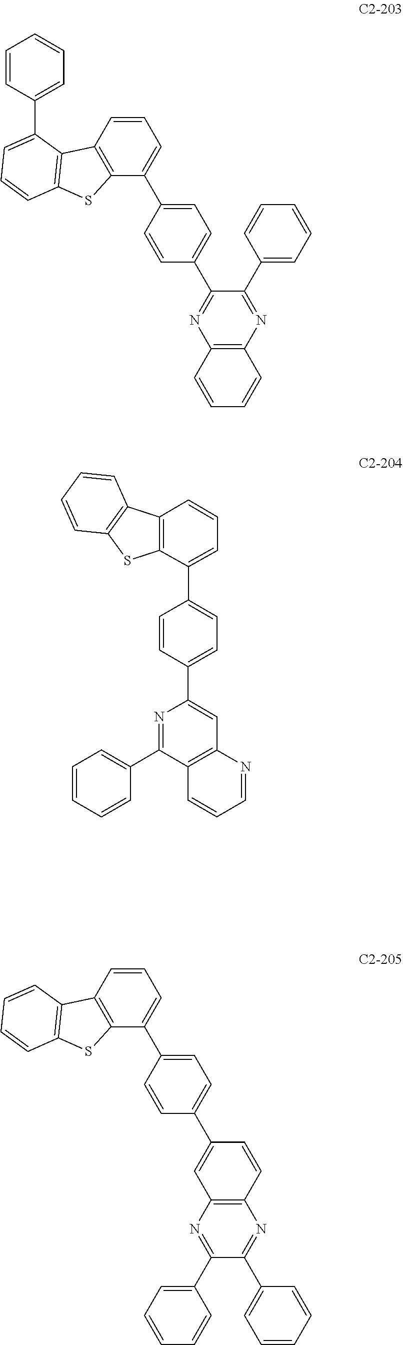 [SMILES: c1ccc(-c2nc3ccccc3nc2-c2ccc(-c3cccc4c3sc3cccc(-c5ccccc5)c34)cc2)cc1 ; c1ccc(-c2nc(-c3ccc(-c4cccc5c4sc4ccccc45)cc3)cc3ncccc23)cc1 ; c1ccc(-c2nc3ccc(-c4ccc(-c5cccc6c5sc5ccccc56)cc4)cc3nc2-c2ccccc2)cc1]

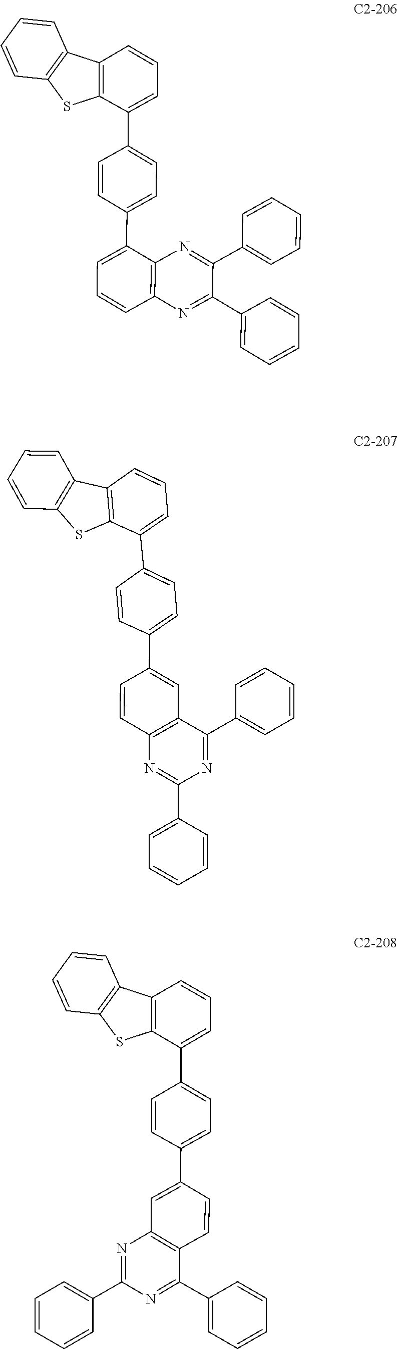 [SMILES: c1ccc(-c2nc3cccc(-c4ccc(-c5cccc6c5sc5ccccc56)cc4)c3nc2-c2ccccc2)cc1 ; c1ccc(-c2nc(-c3ccccc3)c3cc(-c4ccc(-c5cccc6c5sc5ccccc56)cc4)ccc3n2)cc1 ; c1ccc(-c2nc(-c3ccccc3)c3ccc(-c4ccc(-c5cccc6c5sc5ccccc56)cc4)cc3n2)cc1]

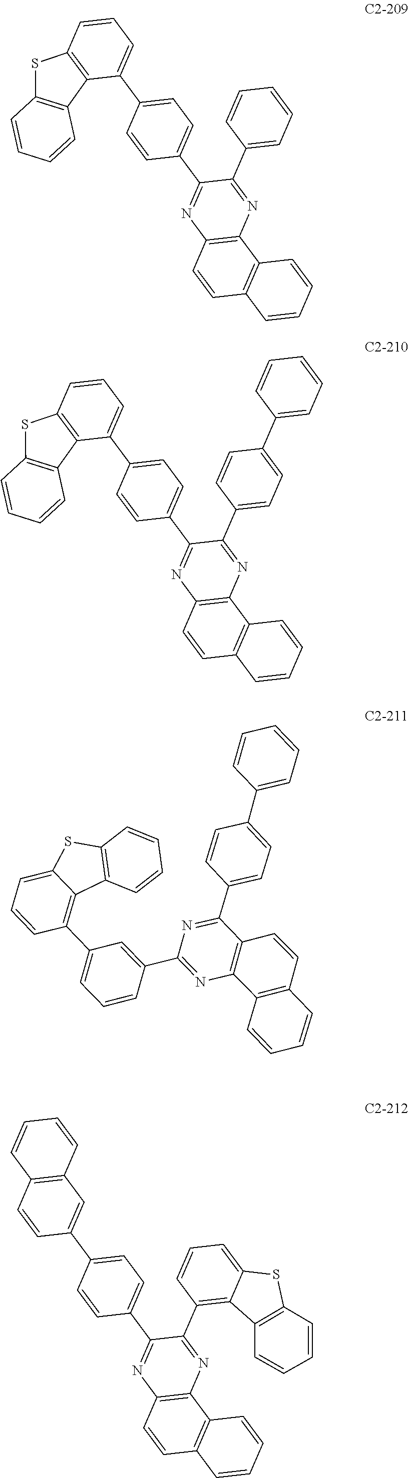

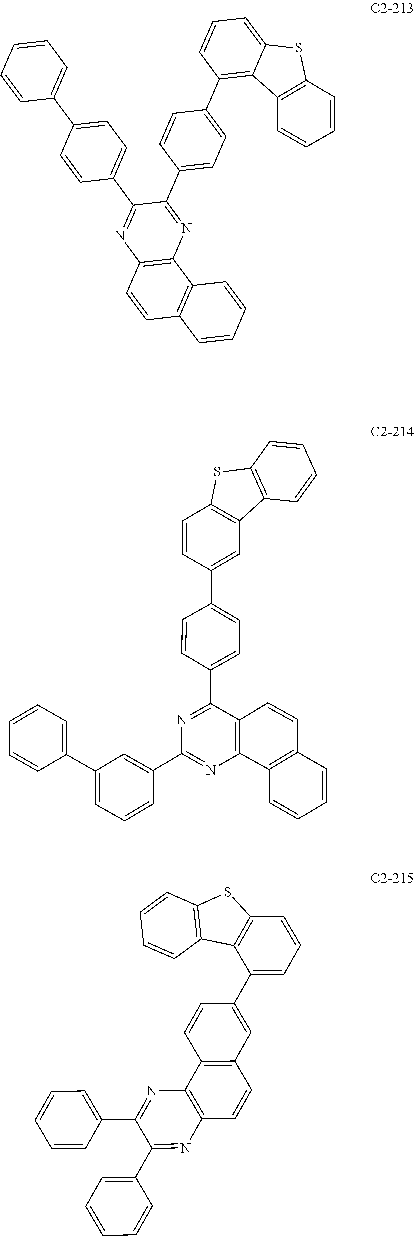 [SMILES: c1ccc(-c2ccc(-c3nc4ccc5ccccc5c4nc3-c3ccc(-c4cccc5sc6ccccc6c45)cc3)cc2)cc1 ; c1ccc(-c2cccc(-c3nc(-c4ccc(-c5ccc6sc7ccccc7c6c5)cc4)c4ccc5ccccc5c4n3)c2)cc1 ; c1ccc(-c2nc3ccc4cc(-c5cccc6sc7ccccc7c56)ccc4c3nc2-c2ccccc2)cc1]

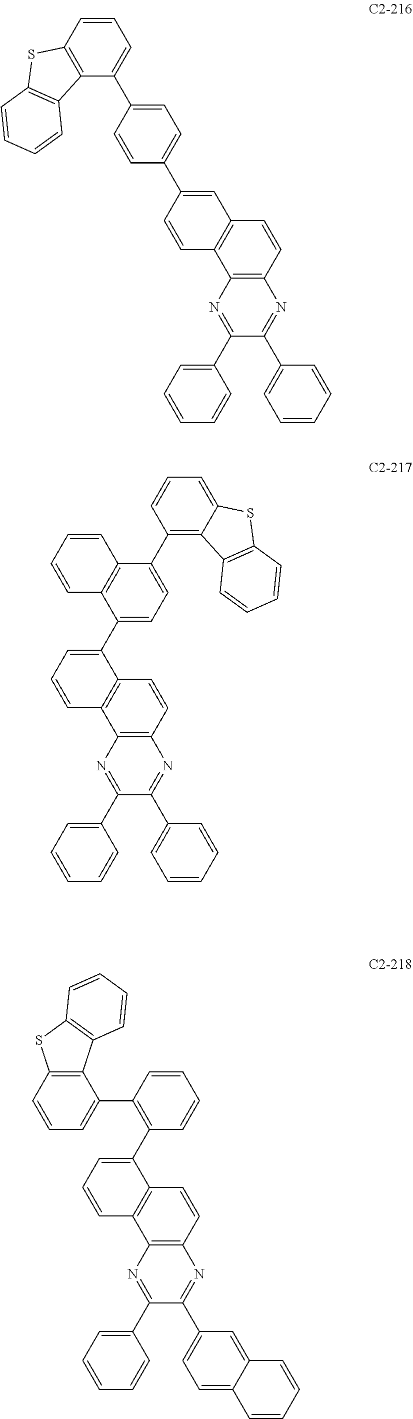 [SMILES: c1ccc(-c2nc3ccc4cc(-c5ccc(-c6cccc7sc8ccccc8c67)cc5)ccc4c3nc2-c2ccccc2)cc1 ; c1ccc(-c2nc3ccc4c(-c5ccc(-c6cccc7sc8ccccc8c67)c6ccccc56)cccc4c3nc2-c2ccccc2)cc1 ; c1ccc(-c2nc3c(ccc4c(-c5ccccc5-c5cccc6sc7ccccc7c56)cccc43)nc2-c2ccc3ccccc3c2)cc1]

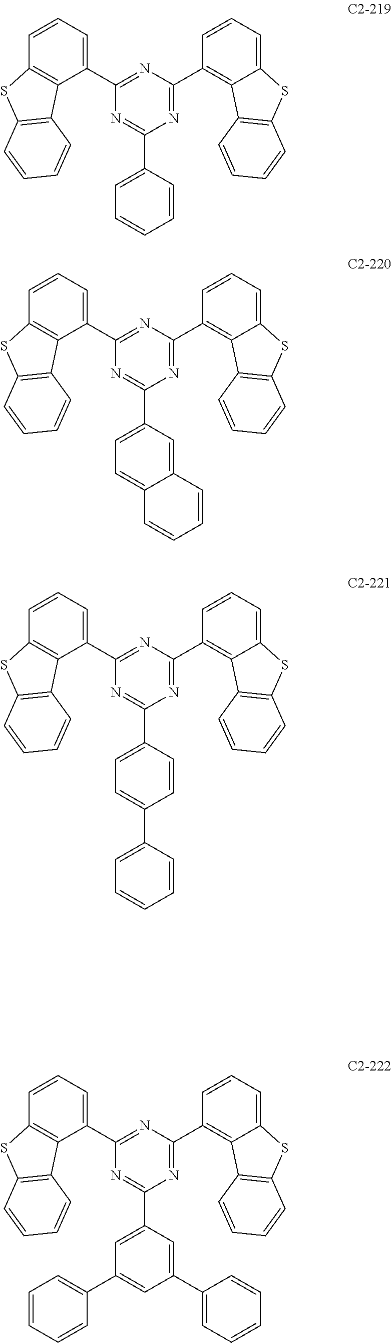 [SMILES: c1ccc(-c2nc(-c3cccc4sc5ccccc5c34)nc(-c3cccc4sc5ccccc5c34)n2)cc1 ; c1ccc2cc(-c3nc(-c4cccc5sc6ccccc6c45)nc(-c4cccc5sc6ccccc6c45)n3)ccc2c1 ; c1ccc(-c2ccc(-c3nc(-c4cccc5sc6ccccc6c45)nc(-c4cccc5sc6ccccc6c45)n3)cc2)cc1 ; c1ccc(-c2cc(-c3ccccc3)cc(-c3nc(-c4cccc5sc6ccccc6c45)nc(-c4cccc5sc6ccccc6c45)n3)c2)cc1]

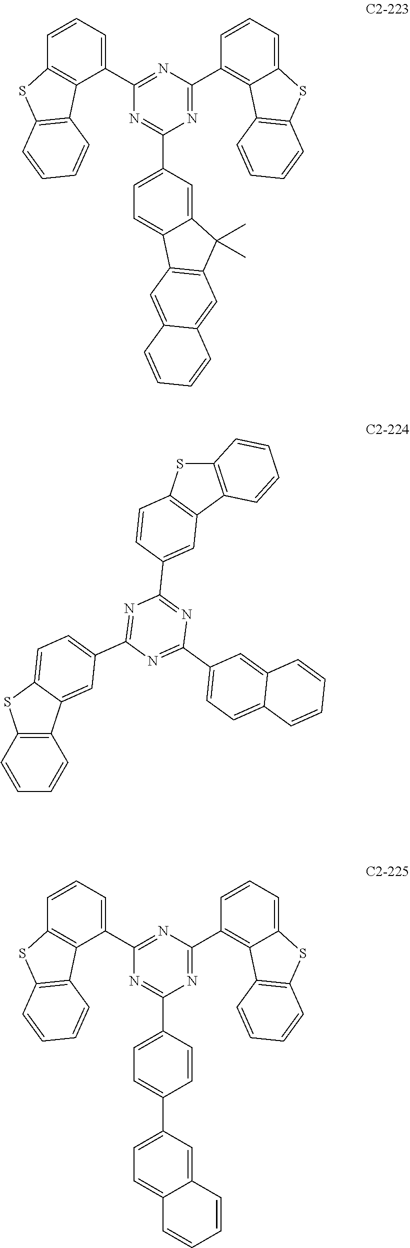 [SMILES: CC1(C)c2cc(-c3nc(-c4cccc5sc6ccccc6c45)nc(-c4cccc5sc6ccccc6c45)n3)ccc2-c2cc3ccccc3cc21 ; c1ccc2cc(-c3nc(-c4ccc5sc6ccccc6c5c4)nc(-c4ccc5sc6ccccc6c5c4)n3)ccc2c1 ; c1ccc2cc(-c3ccc(-c4nc(-c5cccc6sc7ccccc7c56)nc(-c5cccc6sc7ccccc7c56)n4)cc3)ccc2c1]

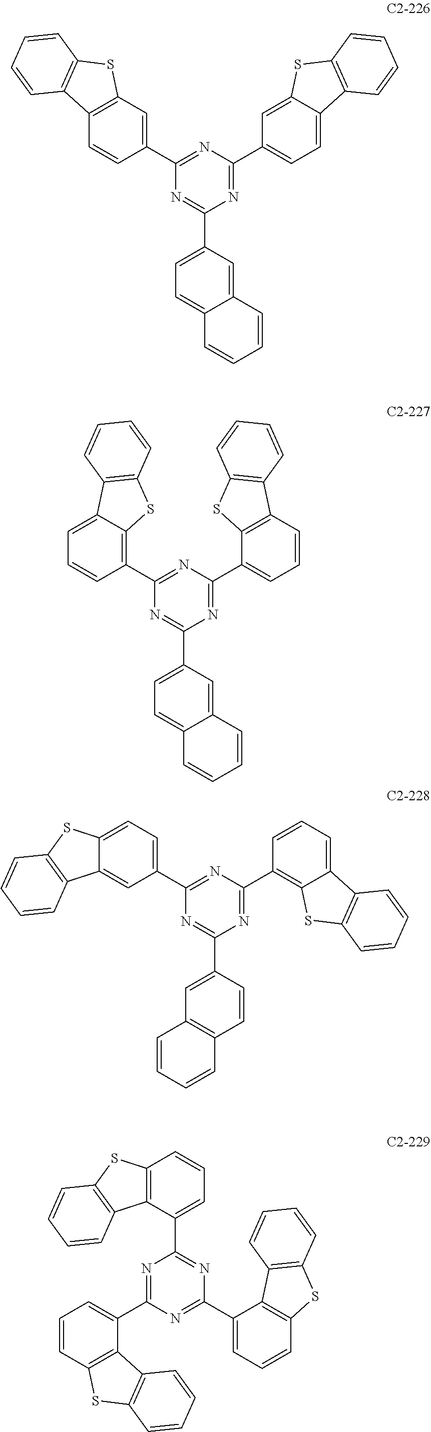 [SMILES: c1ccc2cc(-c3nc(-c4ccc5c(c4)sc4ccccc45)nc(-c4ccc5c(c4)sc4ccccc45)n3)ccc2c1 ; c1ccc2cc(-c3nc(-c4cccc5c4sc4ccccc45)nc(-c4cccc5c4sc4ccccc45)n3)ccc2c1 ; c1ccc2cc(-c3nc(-c4ccc5sc6ccccc6c5c4)nc(-c4cccc5c4sc4ccccc45)n3)ccc2c1 ; c1ccc2c(c1)sc1cccc(-c3nc(-c4cccc5sc6ccccc6c45)nc(-c4cccc5sc6ccccc6c45)n3)c12]

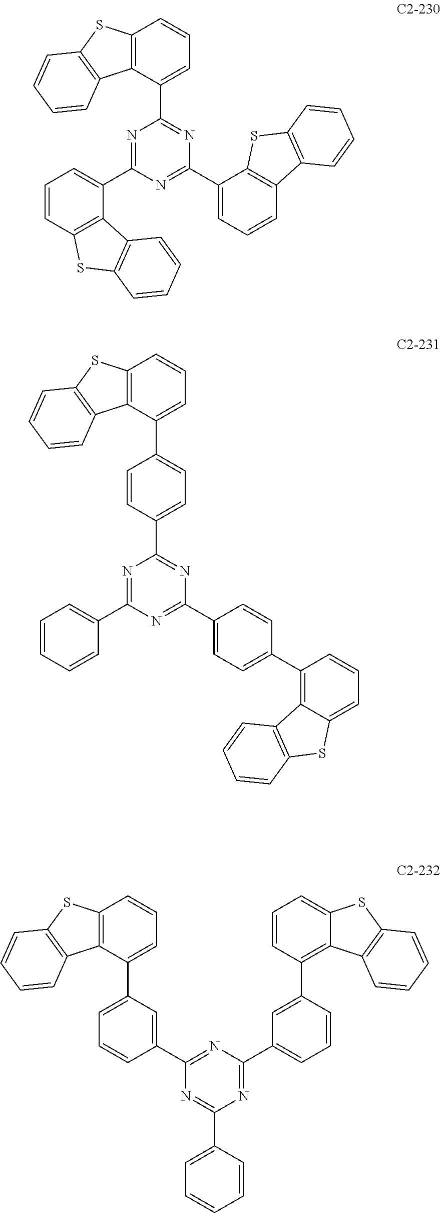 [SMILES: c1ccc2c(c1)sc1c(-c3nc(-c4cccc5sc6ccccc6c45)nc(-c4cccc5sc6ccccc6c45)n3)cccc12 ; c1ccc(-c2nc(-c3ccc(-c4cccc5sc6ccccc6c45)cc3)nc(-c3ccc(-c4cccc5sc6ccccc6c45)cc3)n2)cc1 ; c1ccc(-c2nc(-c3cccc(-c4cccc5sc6ccccc6c45)c3)nc(-c3cccc(-c4cccc5sc6ccccc6c45)c3)n2)cc1]

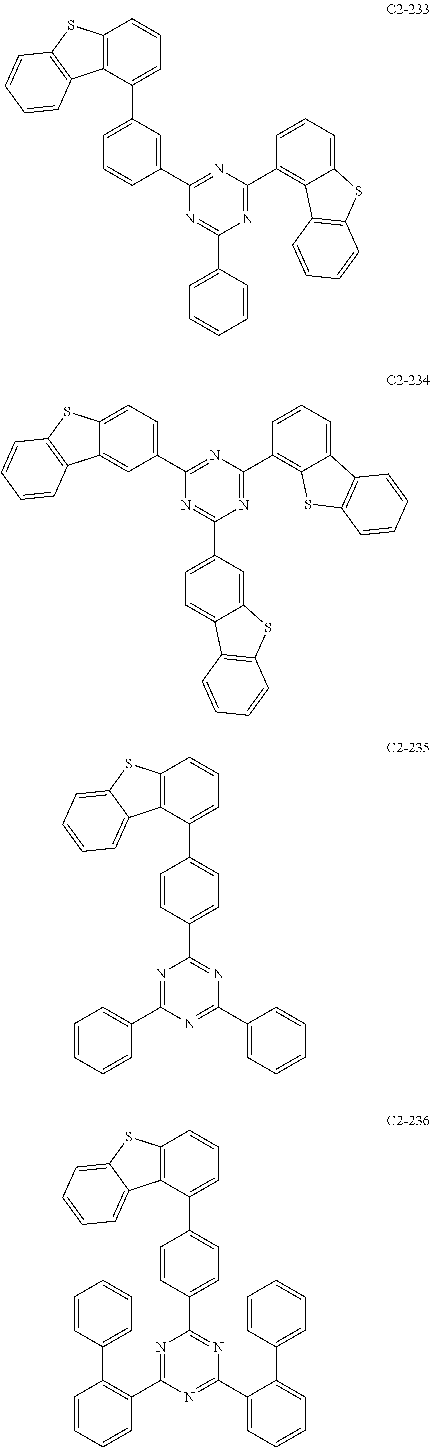 [SMILES: c1ccc(-c2nc(-c3cccc(-c4cccc5sc6ccccc6c45)c3)nc(-c3cccc4sc5ccccc5c34)n2)cc1 ; c1ccc2c(c1)sc1cc(-c3nc(-c4ccc5sc6ccccc6c5c4)nc(-c4cccc5c4sc4ccccc45)n3)ccc12 ; c1ccc(-c2nc(-c3ccccc3)nc(-c3ccc(-c4cccc5sc6ccccc6c45)cc3)n2)cc1 ; c1ccc(-c2ccccc2-c2nc(-c3ccc(-c4cccc5sc6ccccc6c45)cc3)nc(-c3ccccc3-c3ccccc3)n2)cc1]

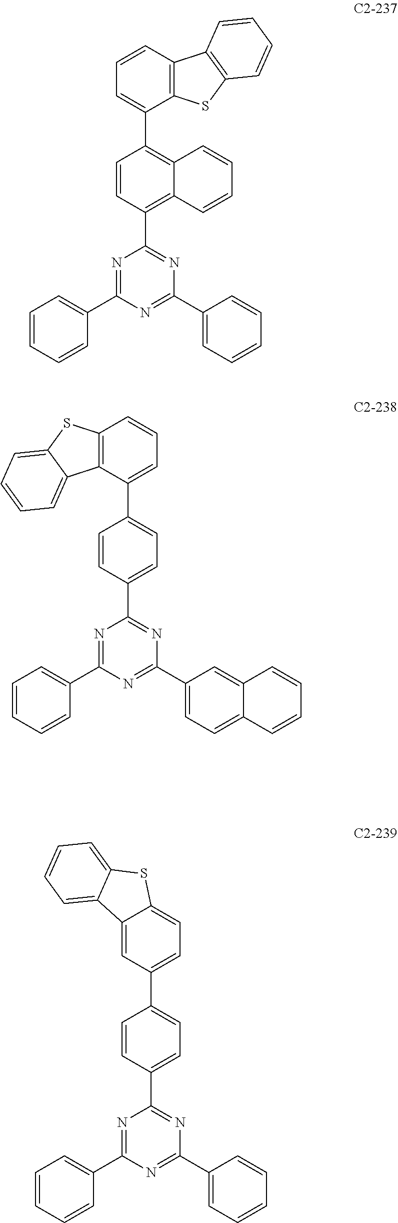 [SMILES: c1ccc(-c2nc(-c3ccccc3)nc(-c3ccc(-c4cccc5c4sc4ccccc45)c4ccccc34)n2)cc1 ; c1ccc(-c2nc(-c3ccc(-c4cccc5sc6ccccc6c45)cc3)nc(-c3ccc4ccccc4c3)n2)cc1 ; c1ccc(-c2nc(-c3ccccc3)nc(-c3ccc(-c4ccc5sc6ccccc6c5c4)cc3)n2)cc1]

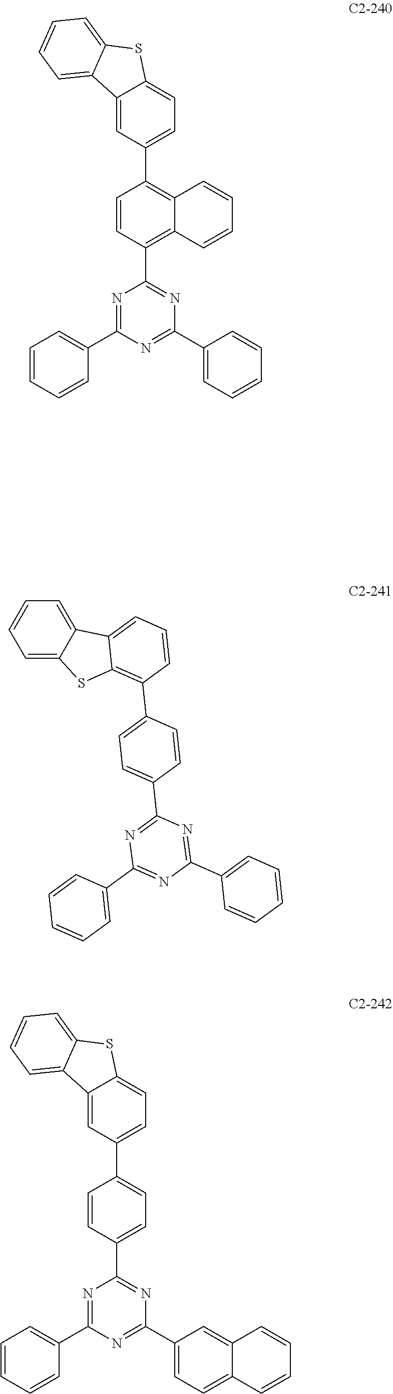 [SMILES: c1ccc(-c2nc(-c3ccccc3)nc(-c3ccc(-c4ccc5sc6ccccc6c5c4)c4ccccc34)n2)cc1 ; c1ccc(-c2nc(-c3ccccc3)nc(-c3ccc(-c4cccc5c4sc4ccccc45)cc3)n2)cc1 ; c1ccc(-c2nc(-c3ccc(-c4ccc5sc6ccccc6c5c4)cc3)nc(-c3ccc4ccccc4c3)n2)cc1]

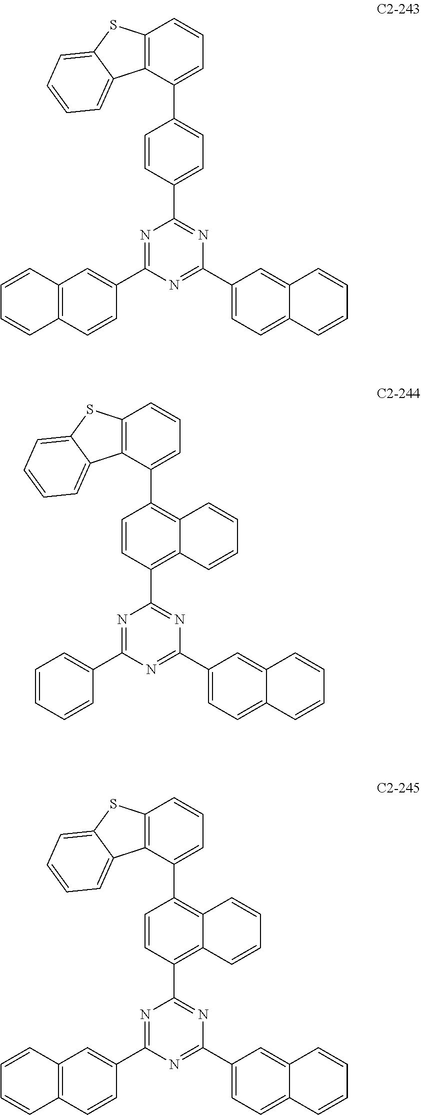 [SMILES: c1ccc2cc(-c3nc(-c4ccc(-c5cccc6sc7ccccc7c56)cc4)nc(-c4ccc5ccccc5c4)n3)ccc2c1 ; c1ccc(-c2nc(-c3ccc4ccccc4c3)nc(-c3ccc(-c4cccc5sc6ccccc6c45)c4ccccc34)n2)cc1 ; c1ccc2cc(-c3nc(-c4ccc5ccccc5c4)nc(-c4ccc(-c5cccc6sc7ccccc7c56)c5ccccc45)n3)ccc2c1]

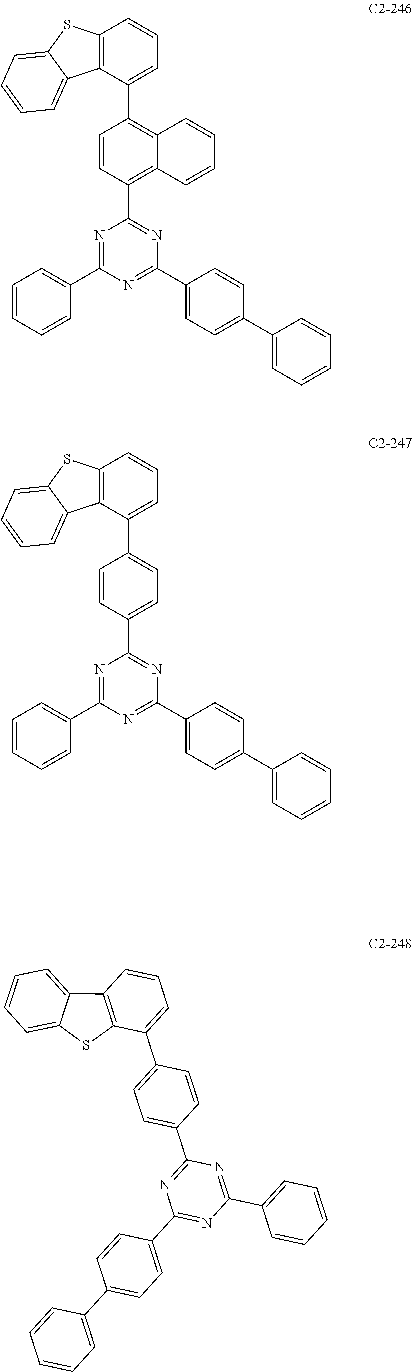 [SMILES: c1ccc(-c2ccc(-c3nc(-c4ccccc4)nc(-c4ccc(-c5cccc6sc7ccccc7c56)c5ccccc45)n3)cc2)cc1 ; c1ccc(-c2ccc(-c3nc(-c4ccccc4)nc(-c4ccc(-c5cccc6sc7ccccc7c56)cc4)n3)cc2)cc1 ; c1ccc(-c2ccc(-c3nc(-c4ccccc4)nc(-c4ccc(-c5cccc6c5sc5ccccc56)cc4)n3)cc2)cc1]

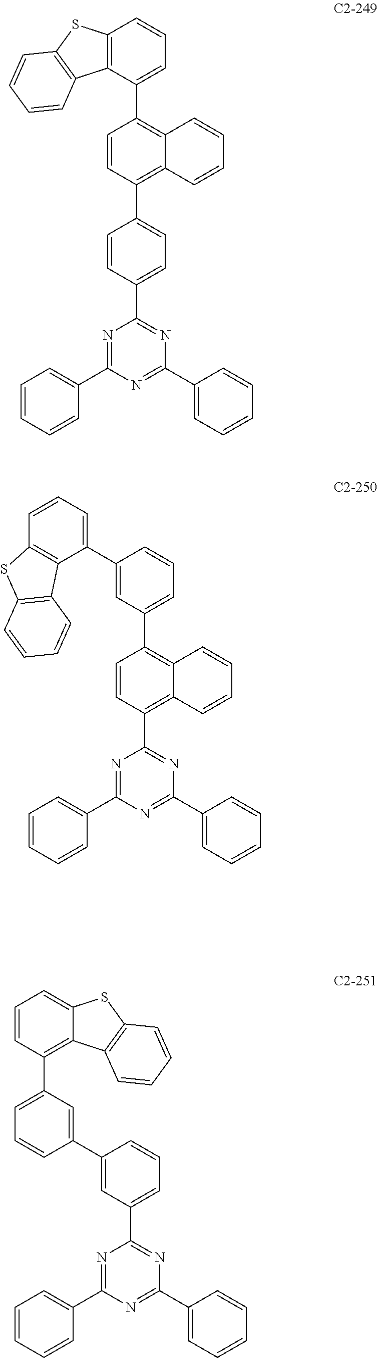 [SMILES: c1ccc(-c2nc(-c3ccccc3)nc(-c3ccc(-c4ccc(-c5cccc6sc7ccccc7c56)c5ccccc45)cc3)n2)cc1 ; c1ccc(-c2nc(-c3ccccc3)nc(-c3ccc(-c4cccc(-c5cccc6sc7ccccc7c56)c4)c4ccccc34)n2)cc1 ; c1ccc(-c2nc(-c3ccccc3)nc(-c3cccc(-c4cccc(-c5cccc6sc7ccccc7c56)c4)c3)n2)cc1]

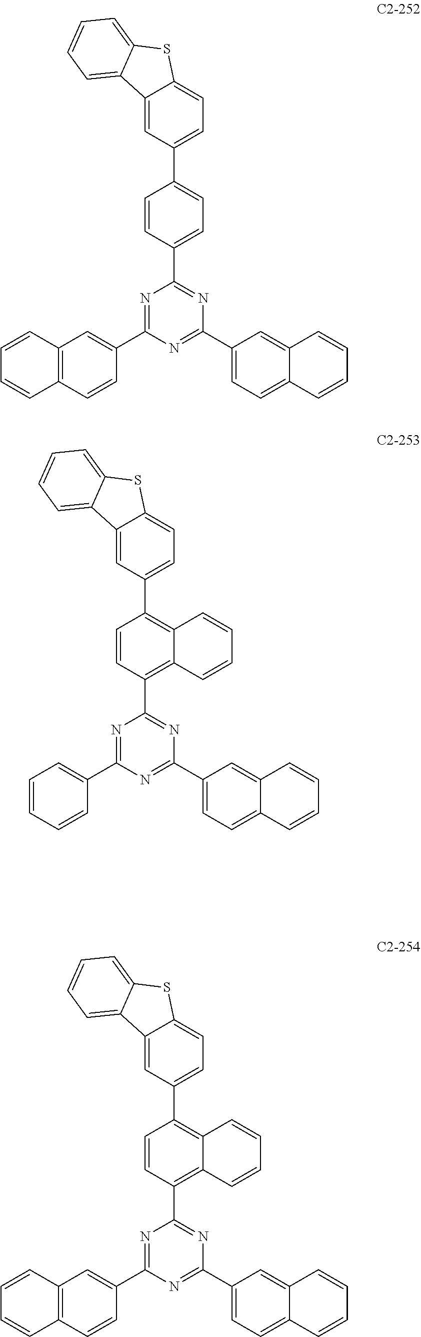 [SMILES: c1ccc2cc(-c3nc(-c4ccc(-c5ccc6sc7ccccc7c6c5)cc4)nc(-c4ccc5ccccc5c4)n3)ccc2c1 ; c1ccc(-c2nc(-c3ccc4ccccc4c3)nc(-c3ccc(-c4ccc5sc6ccccc6c5c4)c4ccccc34)n2)cc1 ; c1ccc2cc(-c3nc(-c4ccc5ccccc5c4)nc(-c4ccc(-c5ccc6sc7ccccc7c6c5)c5ccccc45)n3)ccc2c1]

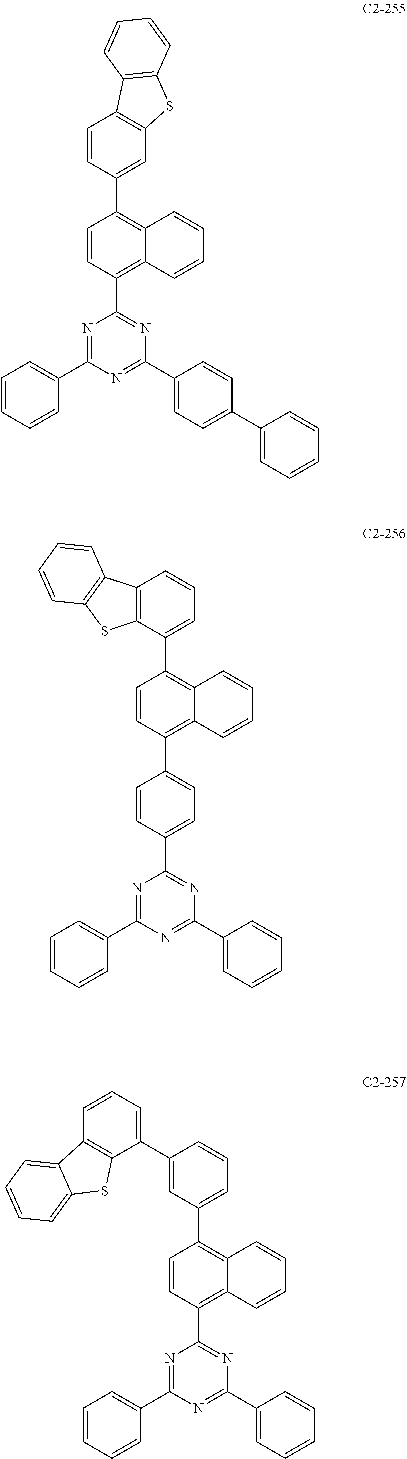 [SMILES: c1ccc(-c2ccc(-c3nc(-c4ccccc4)nc(-c4ccc(-c5ccc6c(c5)sc5ccccc56)c5ccccc45)n3)cc2)cc1 ; c1ccc(-c2nc(-c3ccccc3)nc(-c3ccc(-c4ccc(-c5cccc6c5sc5ccccc56)c5ccccc45)cc3)n2)cc1 ; c1ccc(-c2nc(-c3ccccc3)nc(-c3ccc(-c4cccc(-c5cccc6c5sc5ccccc56)c4)c4ccccc34)n2)cc1]

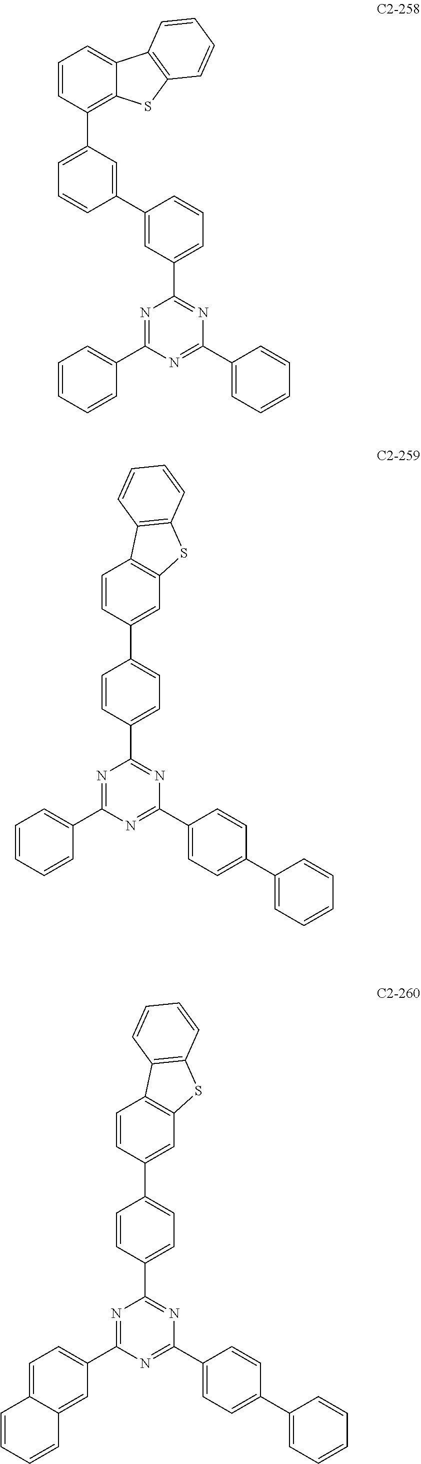 [SMILES: c1ccc(-c2nc(-c3ccccc3)nc(-c3cccc(-c4cccc(-c5cccc6c5sc5ccccc56)c4)c3)n2)cc1 ; c1ccc(-c2ccc(-c3nc(-c4ccccc4)nc(-c4ccc(-c5ccc6c(c5)sc5ccccc56)cc4)n3)cc2)cc1 ; c1ccc(-c2ccc(-c3nc(-c4ccc(-c5ccc6c(c5)sc5ccccc56)cc4)nc(-c4ccc5ccccc5c4)n3)cc2)cc1]

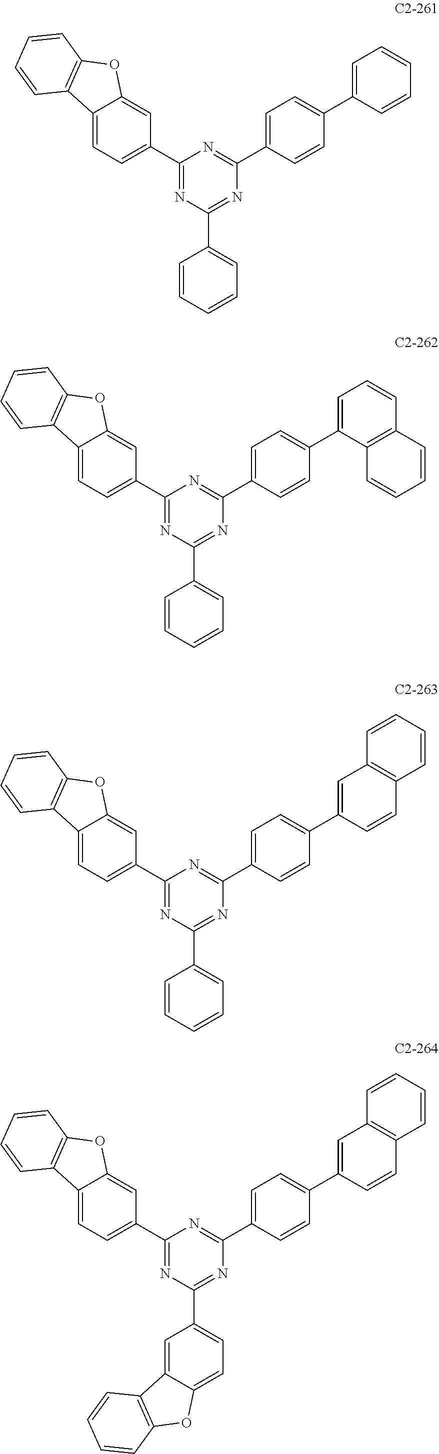 [SMILES: c1ccc(-c2ccc(-c3nc(-c4ccccc4)nc(-c4ccc5c(c4)oc4ccccc45)n3)cc2)cc1 ; c1ccc(-c2nc(-c3ccc(-c4cccc5ccccc45)cc3)nc(-c3ccc4c(c3)oc3ccccc34)n2)cc1 ; c1ccc(-c2nc(-c3ccc(-c4ccc5ccccc5c4)cc3)nc(-c3ccc4c(c3)oc3ccccc34)n2)cc1 ; c1ccc2cc(-c3ccc(-c4nc(-c5ccc6c(c5)oc5ccccc56)nc(-c5ccc6oc7ccccc7c6c5)n4)cc3)ccc2c1]

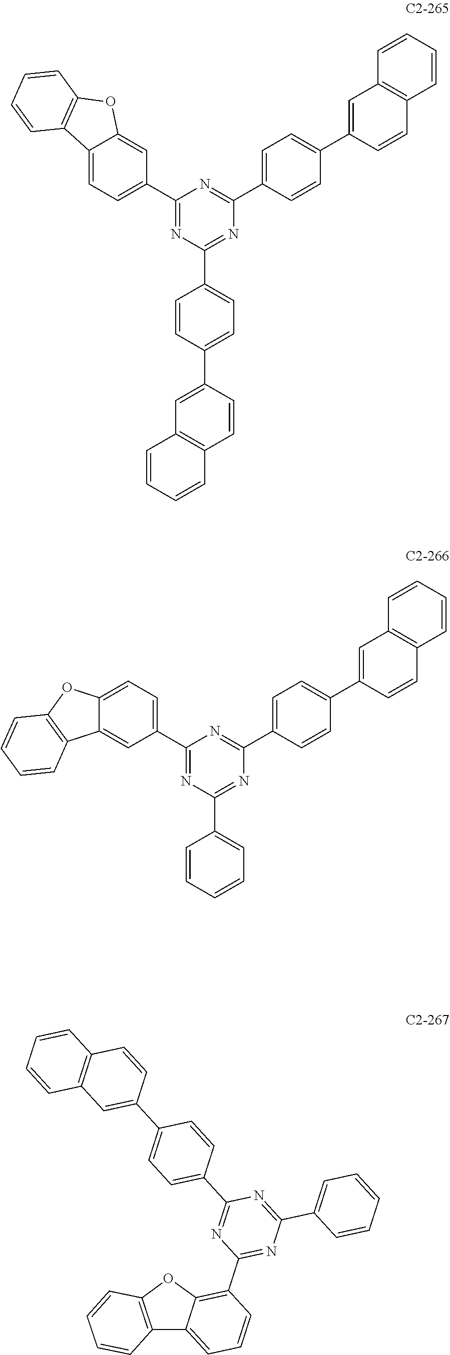 [SMILES: c1ccc2cc(-c3ccc(-c4nc(-c5ccc(-c6ccc7ccccc7c6)cc5)nc(-c5ccc6c(c5)oc5ccccc56)n4)cc3)ccc2c1 ; c1ccc(-c2nc(-c3ccc(-c4ccc5ccccc5c4)cc3)nc(-c3ccc4oc5ccccc5c4c3)n2)cc1 ; c1ccc(-c2nc(-c3ccc(-c4ccc5ccccc5c4)cc3)nc(-c3cccc4c3oc3ccccc34)n2)cc1]

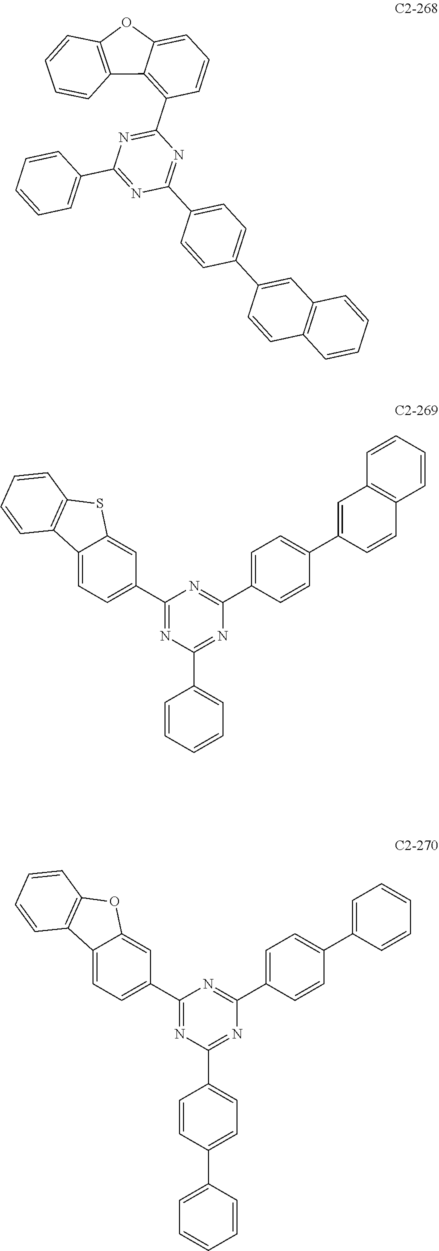 [SMILES: c1ccc(-c2nc(-c3ccc(-c4ccc5ccccc5c4)cc3)nc(-c3cccc4oc5ccccc5c34)n2)cc1 ; c1ccc(-c2nc(-c3ccc(-c4ccc5ccccc5c4)cc3)nc(-c3ccc4c(c3)sc3ccccc34)n2)cc1 ; c1ccc(-c2ccc(-c3nc(-c4ccc(-c5ccccc5)cc4)nc(-c4ccc5c(c4)oc4ccccc45)n3)cc2)cc1]

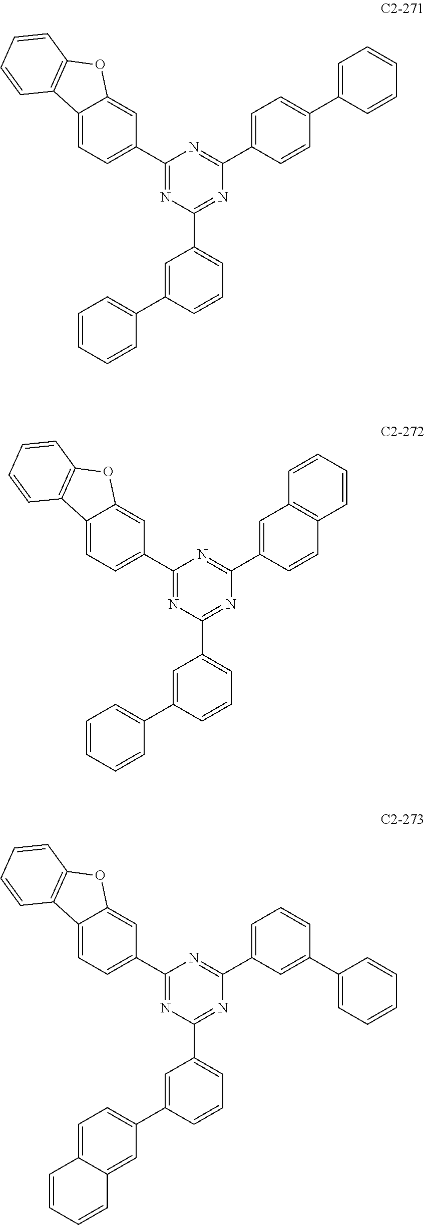 [SMILES: c1ccc(-c2ccc(-c3nc(-c4cccc(-c5ccccc5)c4)nc(-c4ccc5c(c4)oc4ccccc45)n3)cc2)cc1 ; c1ccc(-c2cccc(-c3nc(-c4ccc5ccccc5c4)nc(-c4ccc5c(c4)oc4ccccc45)n3)c2)cc1 ; c1ccc(-c2cccc(-c3nc(-c4cccc(-c5ccc6ccccc6c5)c4)nc(-c4ccc5c(c4)oc4ccccc45)n3)c2)cc1]

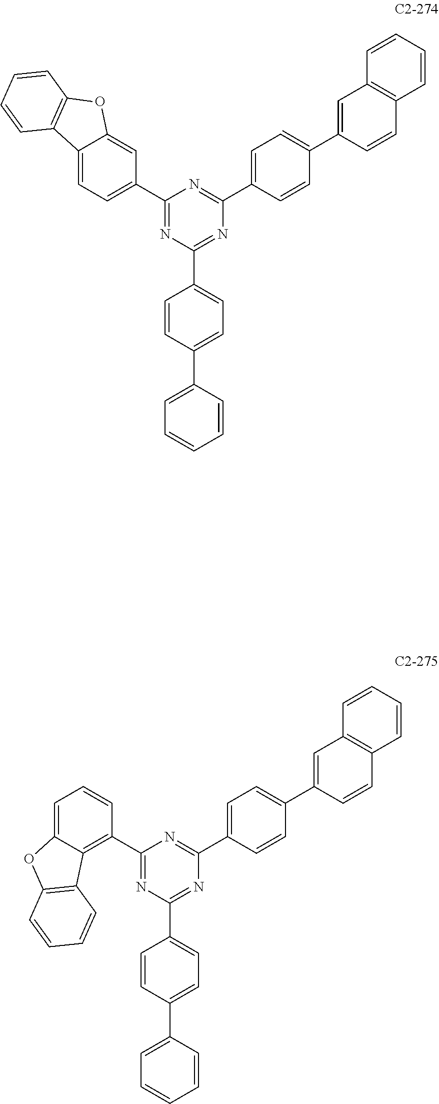 [SMILES: c1ccc(-c2ccc(-c3nc(-c4ccc(-c5ccc6ccccc6c5)cc4)nc(-c4ccc5c(c4)oc4ccccc45)n3)cc2)cc1 ; c1ccc(-c2ccc(-c3nc(-c4ccc(-c5ccc6ccccc6c5)cc4)nc(-c4cccc5oc6ccccc6c45)n3)cc2)cc1]

The compound represented by the formula 3 according to one embodiment may be produced with reference to a synthetic method known to a person skilled in the art.

Hereinafter, an organic electroluminescent device to which the aforementioned organic electroluminescent compound and/or the plurality of host materials is(are) applied will be described.

The organic electroluminescent device according to one embodiment includes a first electrode; a second electrode; and at least one organic layer(s) interposed between the first electrode and the second electrode. The organic layer may include a light-emitting layer, and the light-emitting layer may comprise organic electroluminescent compound represented by the formula 1. The organic electroluminescent device according to another embodiment of the present disclosure may include a plurality of host materials comprising at least one first host material represented by the formula 1 and at least one second host material represented by the formula 2 and/or the formula 3.

According to one embodiment, the organic electroluminescent material of the present disclosure comprises at least one compound(s) of compounds C-1 to C-410 represented by the formula 1 alone or in combination of two or more, and the organic electroluminescent material may be included in the organic layer, e.g., a light-emitting layer, of the organic electroluminescent device.

According to one embodiment, the organic electroluminescent material of the present disclosure comprises at least one compound(s) of compounds C-1 to C-410 as the first host material and at least one compound(s) of compounds H1-1 to H1-131 as the second host material represented by the formula 2 and/or at least one compound(s) of compounds C2-1 to C2-275 as the second host material represented by the formula 3, and the plurality of host materials may be included in the same organic layer, for example, a light-emitting layer, or may be included in different light-emitting layers, respectively.

The organic layer may further comprise at least one layer selected from a hole injection layer, a hole transport layer, a hole auxiliary layer, a light-emitting auxiliary layer, an electron transport layer, an electron injection layer, an interlayer, a hole blocking layer, an electron blocking layer, and an electron buffer layer, in addition to the light-emitting layer. The organic layer may further comprise an amine-based compound and/or an azine-based compound other than the light-emitting material according to the present disclosure. Specifically, the hole injection layer, the hole transport layer, the hole auxiliary layer, the light-emitting layer, the light-emitting auxiliary layer, or the electron blocking layer may contain the amine-based compound, e.g., an arylamine-based compound and a styrylarylamine-based compound, etc., as a hole injection material, a hole transport material, a hole auxiliary material, a light-emitting material, a light-emitting auxiliary material, and an electron blocking material. Also, the electron transport layer, the electron injection layer, the electron buffer layer, and the hole blocking layer may contain the azine-based compound as an electron transport material, an electron injection material, an electron buffer material, and a hole blocking material. Also, the organic layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising such a metal.

A plurality of host materials according to one embodiment may be used as light-emitting materials for a white organic light-emitting device. The white organic light-emitting device has suggested various structures such as a parallel side-by-side arrangement method, a stacking arrangement method, or CCM (color conversion material) method, etc., according to the arrangement of R (Red), G (Green), YG (yellowish green), or B (blue) light-emitting units. In addition, the organic electroluminescent material according to one embodiment may also be applied to the organic electroluminescent device comprising a QD (quantum dot).

One of either the first electrode or the second electrode may be an anode and the other may be a cathode. Wherein, the first electrode and the second electrode may each be formed as a transmissive conductive material, a transflective conductive material, or a reflective conductive material. The organic electroluminescent device may be a top emission type, a bottom emission type, or a both-sides emission type according to the kinds of the material forming the first electrode and the second electrode.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. Also, the hole injection layer may be doped as a p-dopant. Also, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or the electron blocking layer may be multi-layers, and wherein each layer may use a plurality of compounds.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer may be placed between the electron transport layer (or electron injection layer) and the light-emitting layer, and blocks the arrival of holes to the cathode, thereby improving the probability of recombination of electrons and holes in the light-emitting layer. The hole blocking layer or the electron transport layer may also be multi-layers, wherein each layer may use a plurality of compounds. Also, the electron injection layer may be doped as an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron blocking layer. The light-emitting auxiliary layer, the hole auxiliary layer, or the electron blocking layer may have an effect of improving the efficiency and/or the lifespan of the organic electroluminescent device.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both electrode(s). Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiOₓ(1≤X≤2), AlOₓ(1≤X≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

Further, in the organic electroluminescent device of the present disclosure, preferably, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. A reductive dopant layer may be employed as a charge generating layer to prepare an organic electroluminescent device having two or more light-emitting layers and emitting white light.

The organic electroluminescent device according to one embodiment may further include at least one dopant in the light-emitting layer.

The dopant comprised in the organic electroluminescent device of the present disclosure may be at least one phosphorescent or fluorescent dopant, preferably a phosphorescent dopant. The phosphorescent dopant material applied to the organic electroluminescent device of the present disclosure is not particularly limited, but may be preferably a metallated complex compound(s) of a metal atom(s) selected from iridium (Ir), osmium (Os), copper (Cu), and platinum (Pt), as necessary; more preferably an ortho-metallated complex compound(s) of a metal atom(s) selected from iridium (Ir), osmium (Os), copper (Cu), and platinum (Pt), as necessary; and even more preferably ortho-metallated iridium complex compound(s), as necessary.

The dopant comprised in the organic electroluminescent device of the present disclosure may use the compound represented by the following formula 101, but is not limited thereto.

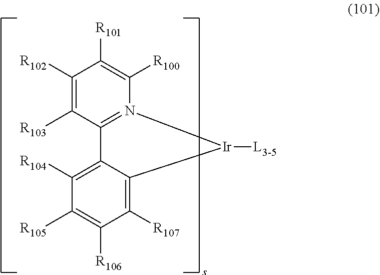

In formula 101,

L is selected from the following structures 1 to 3:

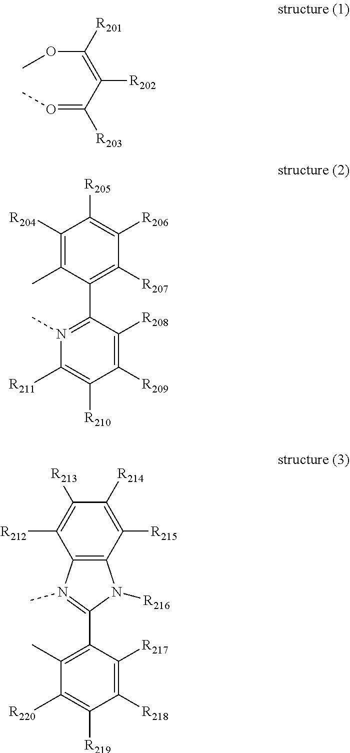

R₁₀₀ to R₁₀₃ each independently represent hydrogen, deuterium, halogen, (C1-C30)alkyl unsubstituted or substituted with deuterium and/or halogen, a substituted or unsubstituted (C3-C30)cycloalkyl, a substituted or unsubstituted (C6-C30)aryl, cyano, a substituted or unsubstituted (3- to 30-membered)heteroaryl, or a substituted or unsubstituted (C1-C30)alkoxy; or the adjacent substituents may be linked to each other to form a ring(s), e.g., a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline, together with pyridine;

R₁₀₄ to R₁₀₇ each independently represent hydrogen, deuterium, halogen, (C1-C30)alkyl unsubstituted or substituted with deuterium and/or halogen, a substituted or unsubstituted (C3-C30)cycloalkyl, a substituted or unsubstituted (C6-C30)aryl, a substituted or unsubstituted (3- to 30-membered)heteroaryl, cyano, or a substituted or unsubstituted (C1-C30)alkoxy; or the adjacent substituents may be linked to each other to form a ring(s), for example, a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine, together with benzene;

R₂₀₁ to R₂₂₀ each independently represent hydrogen, deuterium, halogen, (C1-C30)alkyl unsubstituted or substituted with deuterium and/or halogen, a substituted or unsubstituted (C3-C30)cycloalkyl, or a substituted or unsubstituted (C6-C30)aryl; or the adjacent substituents may be linked to each other to form a substituted or unsubstituted ring(s); and

s represents an integer of 1 to 3. Specifically, the specific examples of the dopant compound include the following, but are not limited thereto.

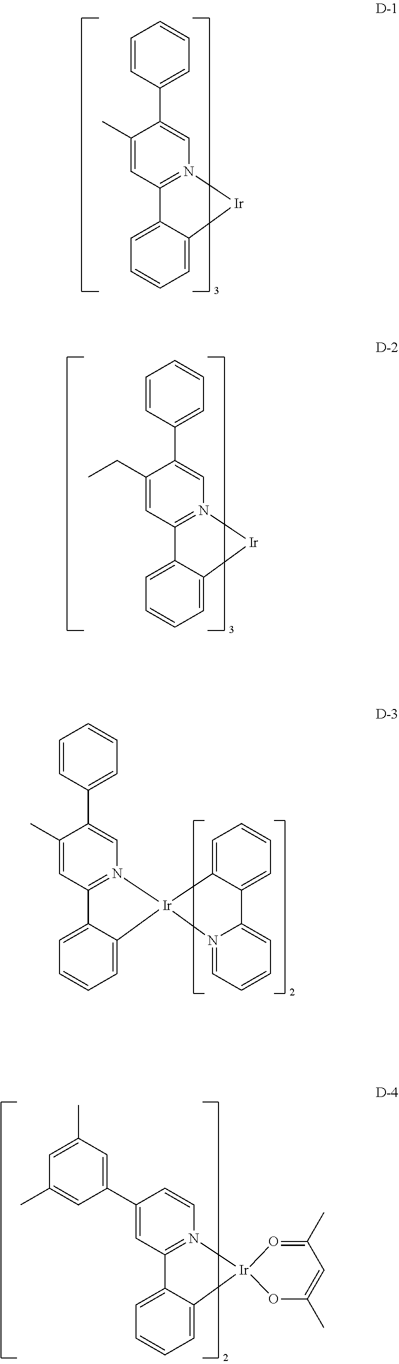

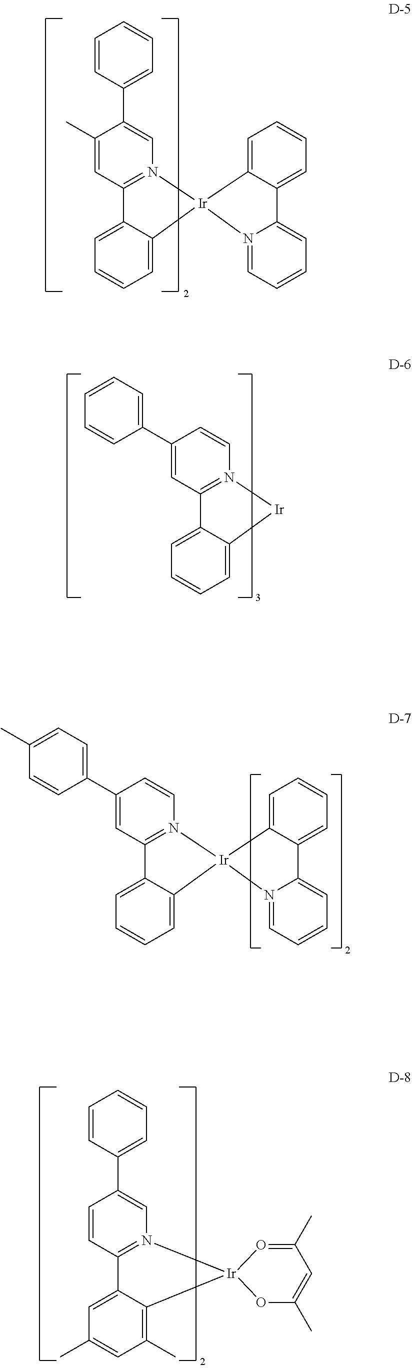

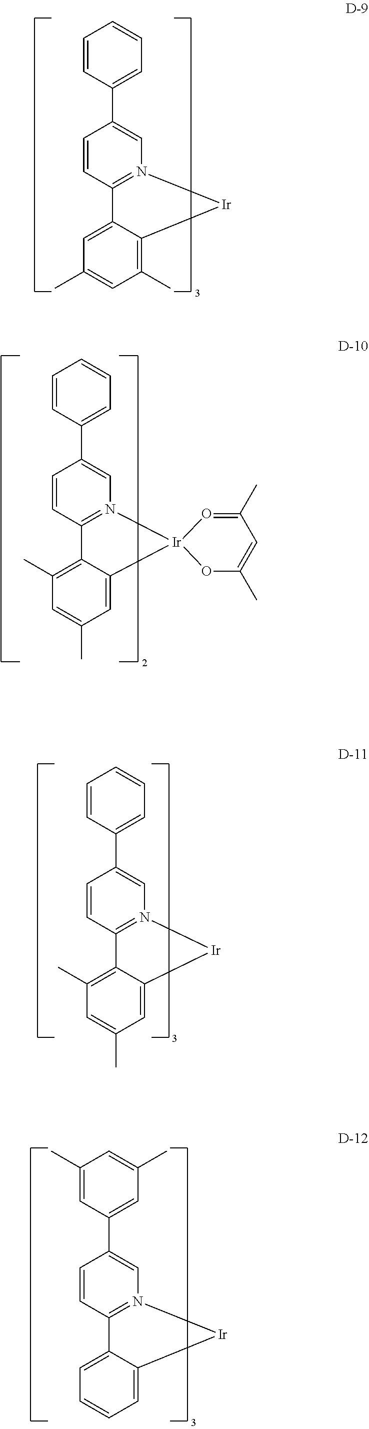

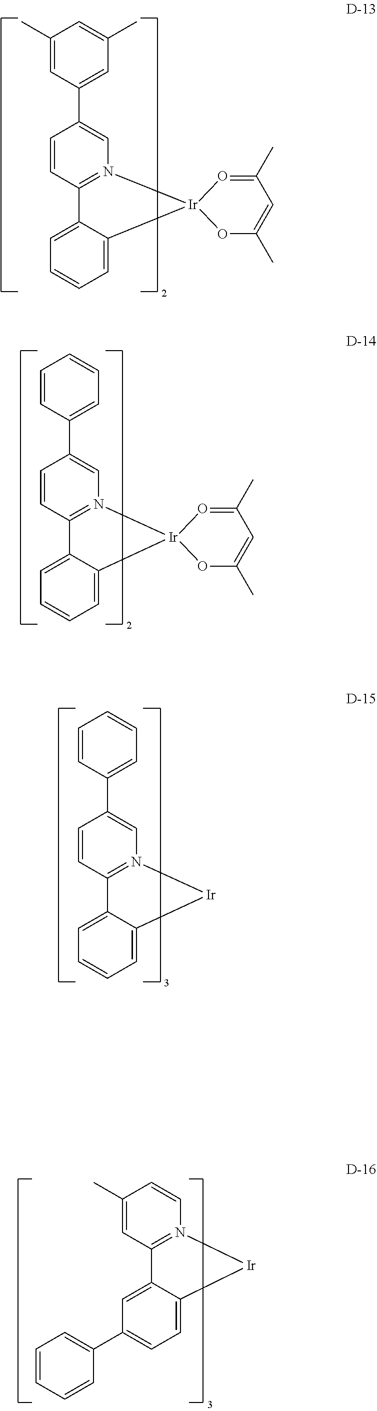

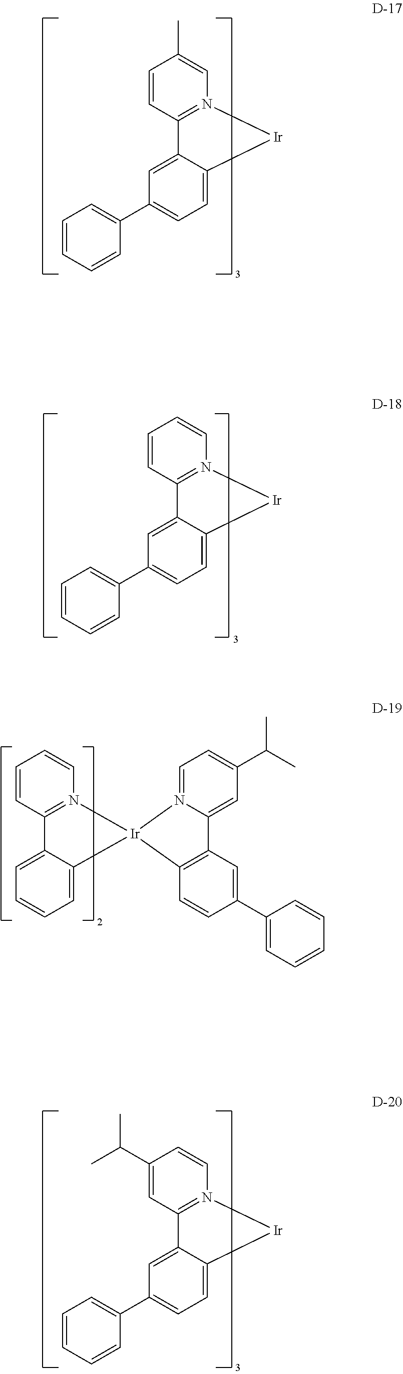

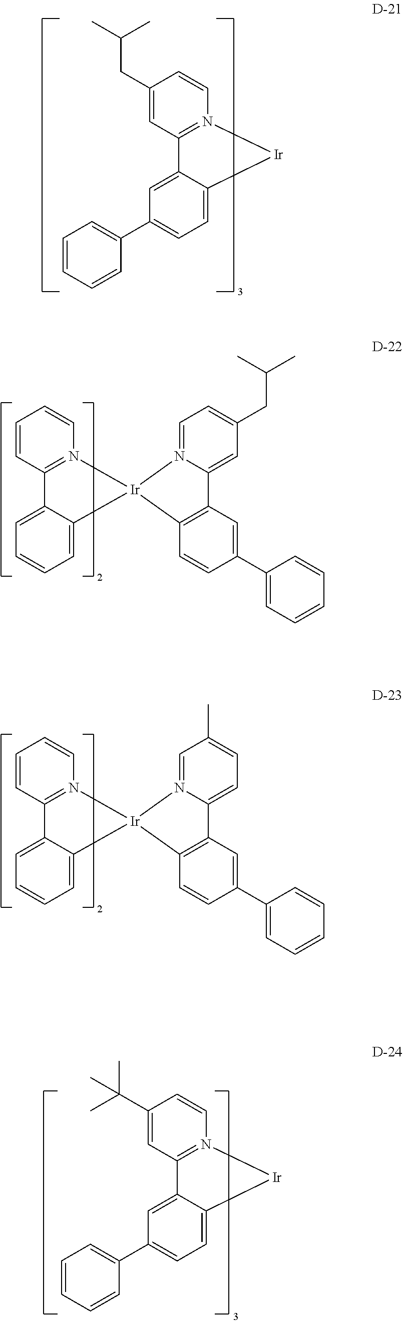

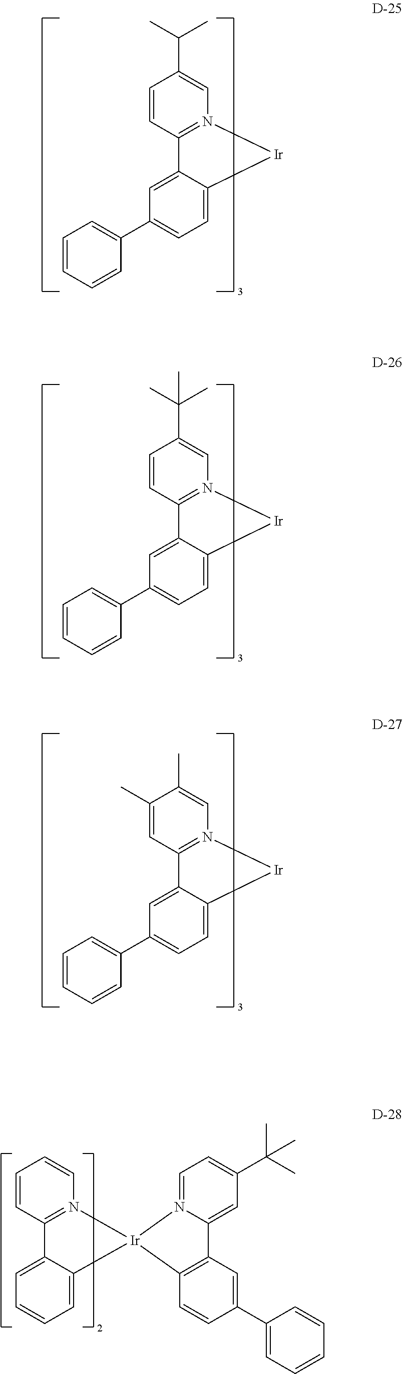

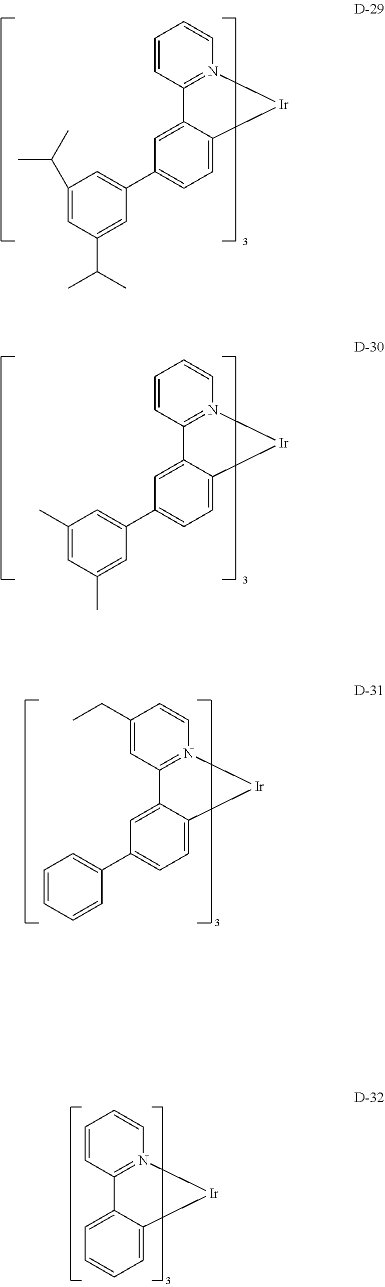

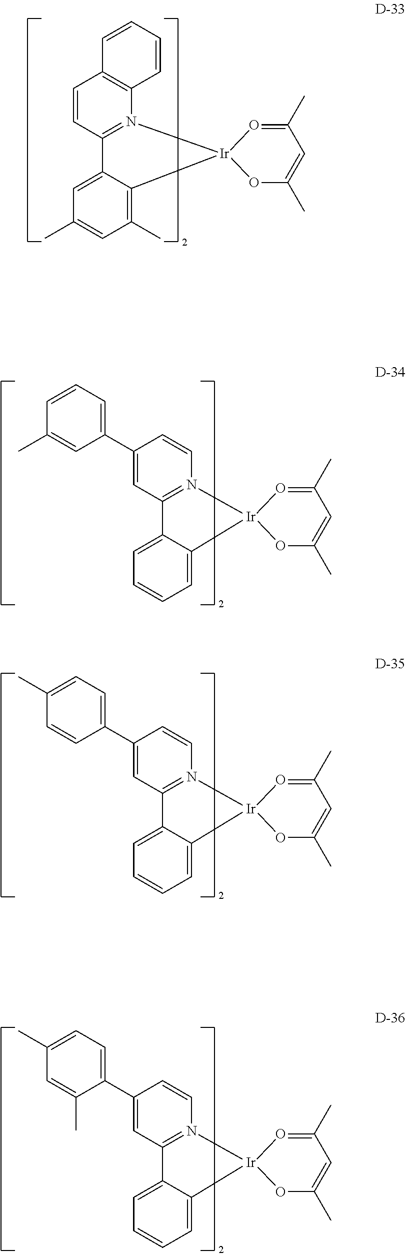

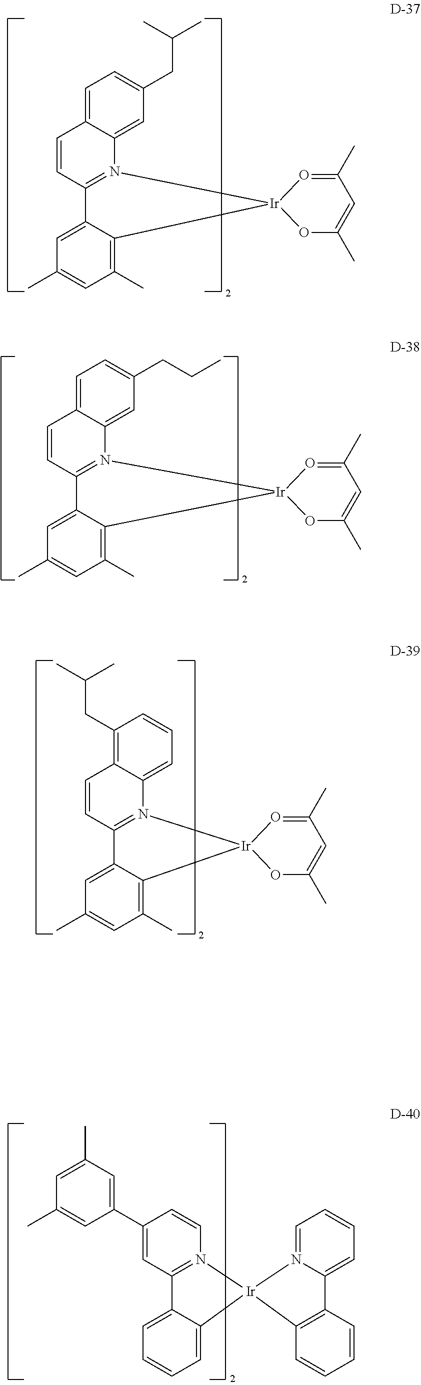

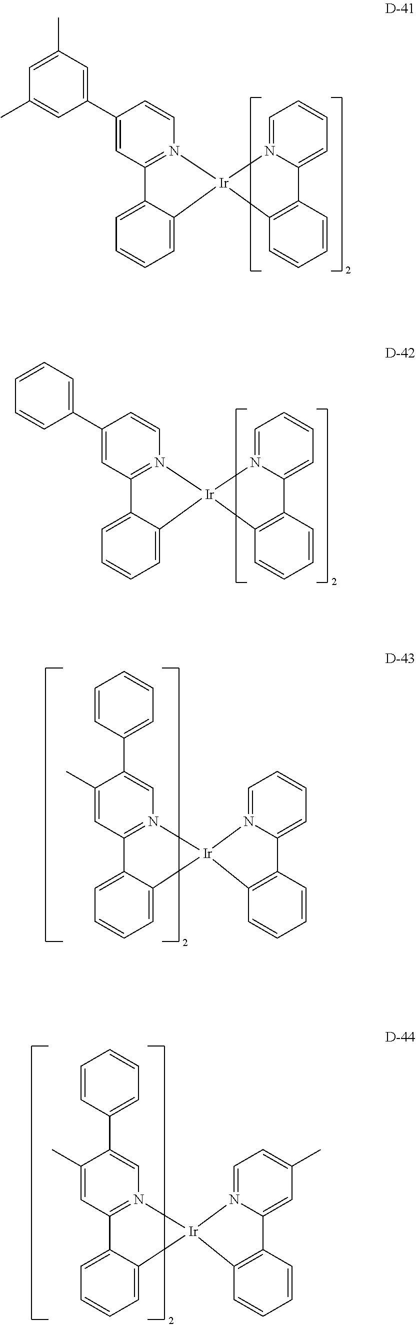

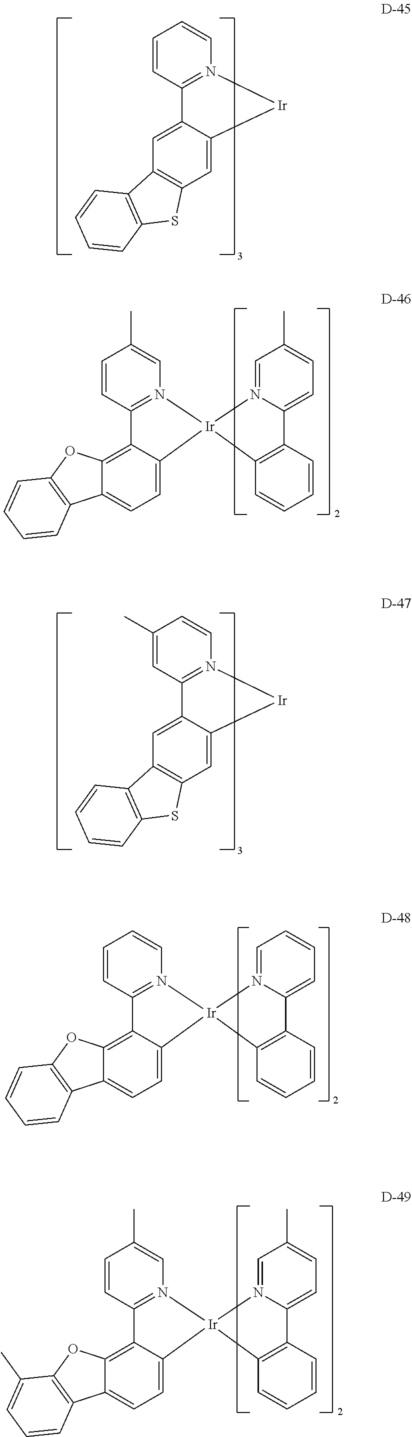

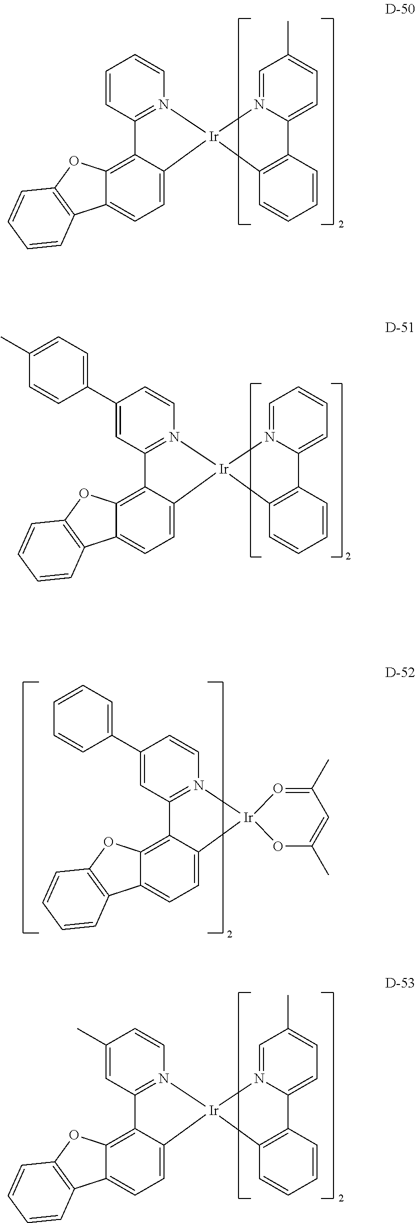

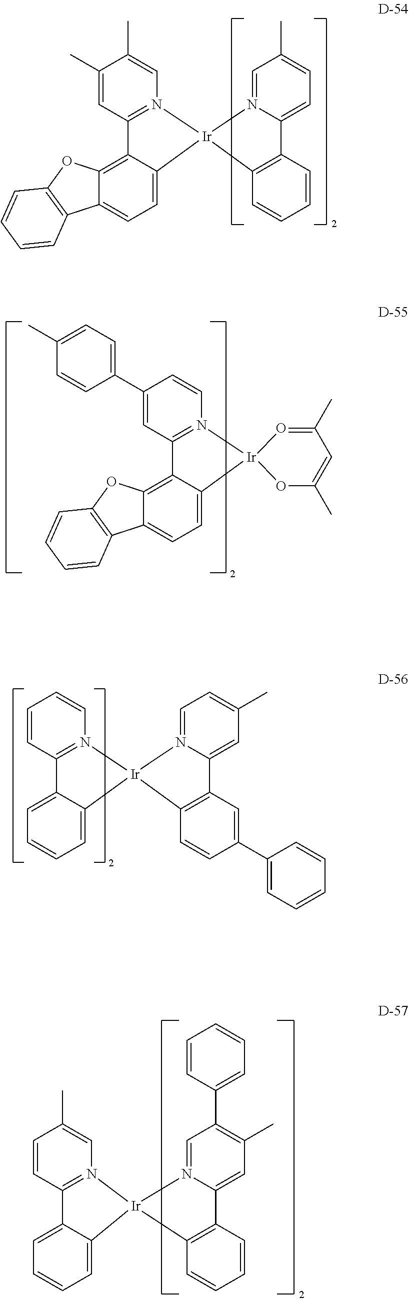

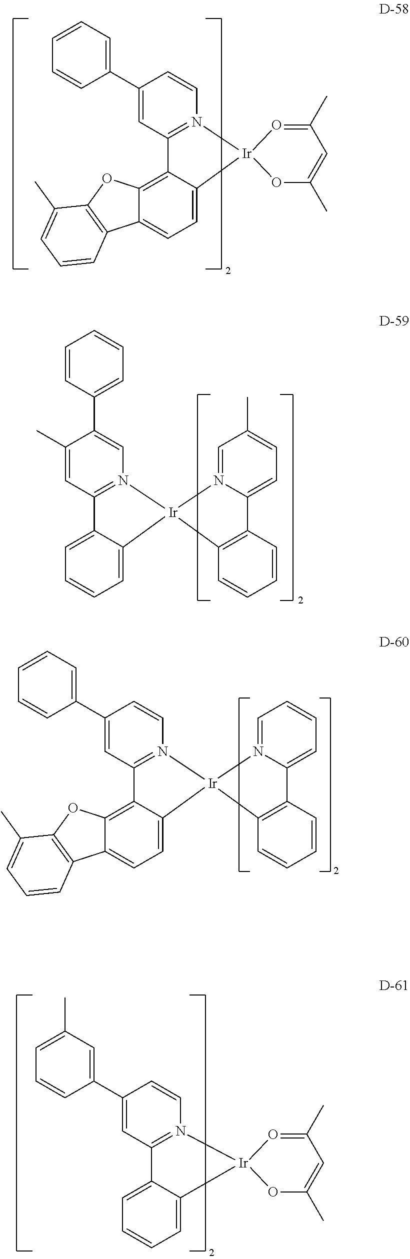

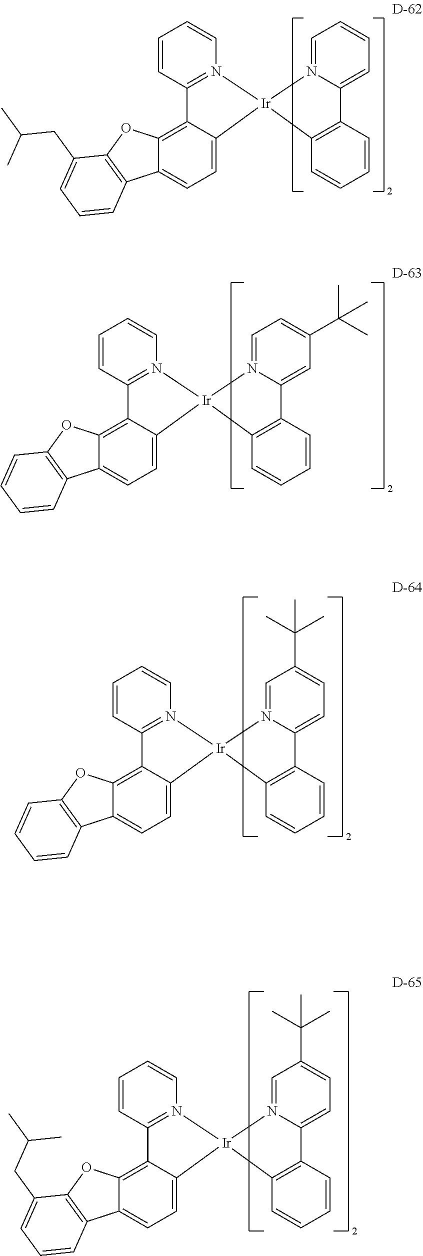

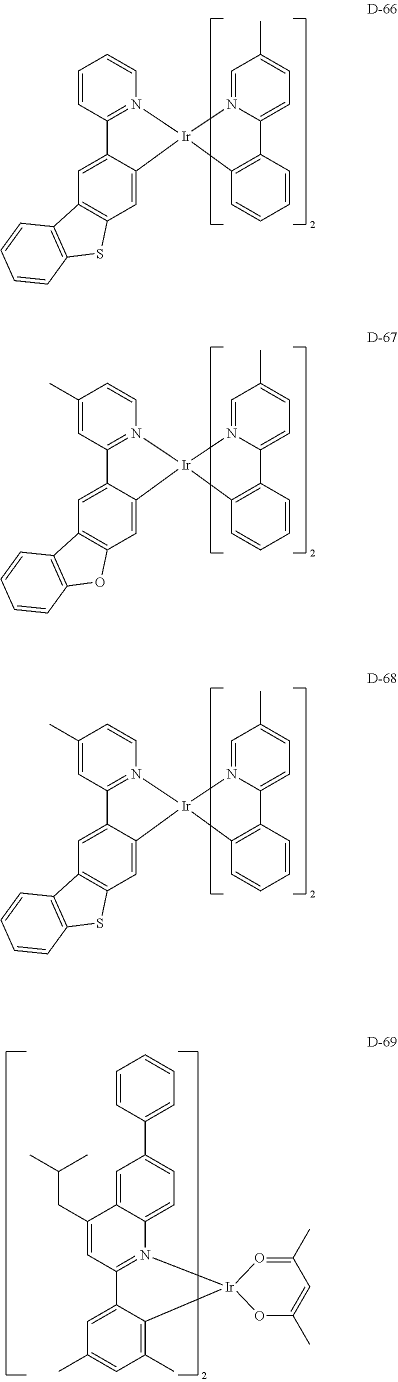

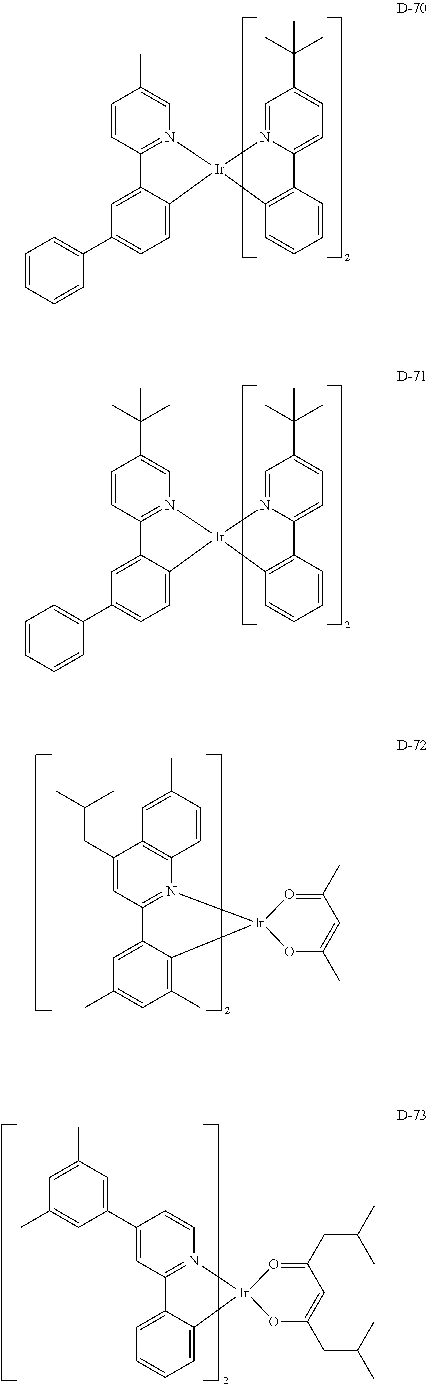

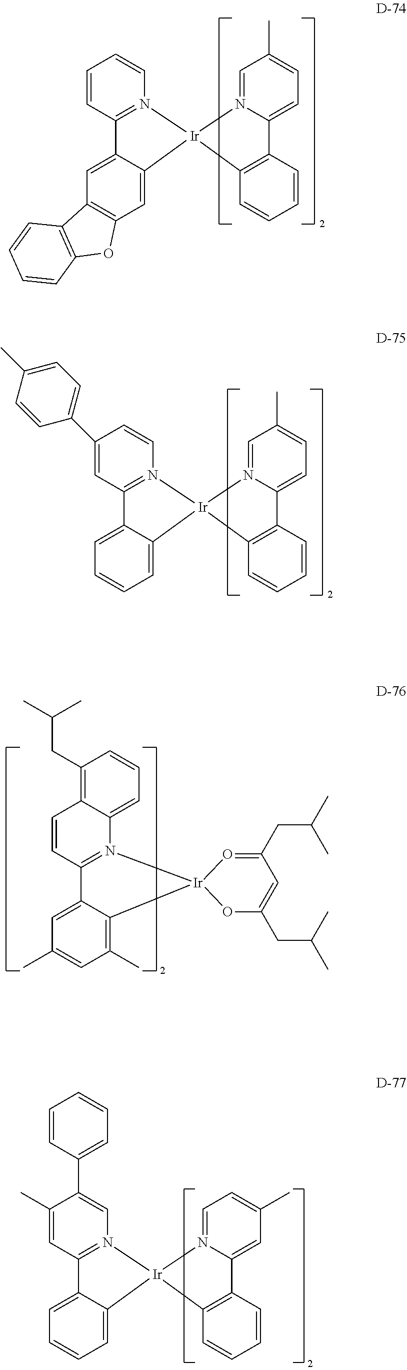

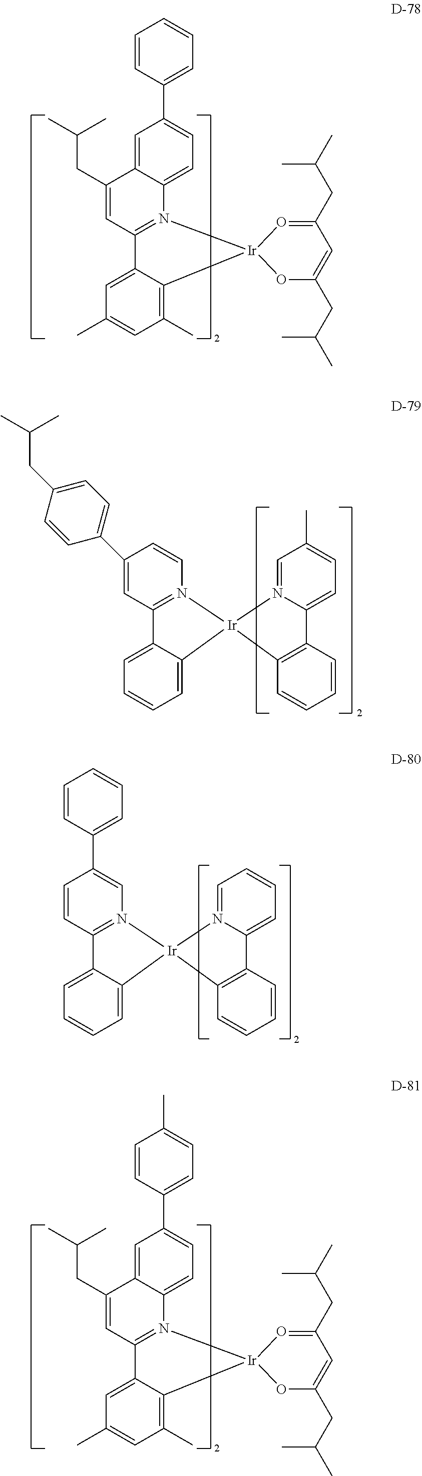

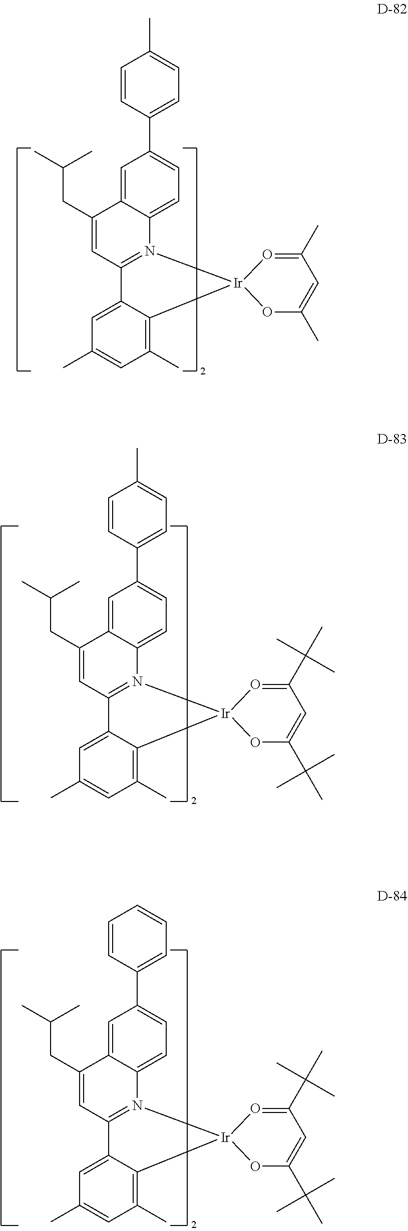

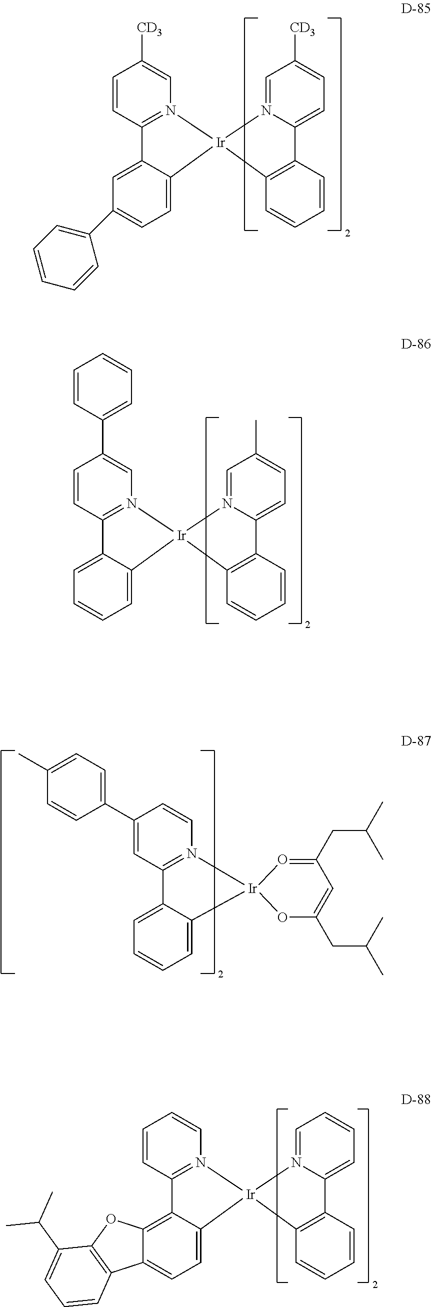

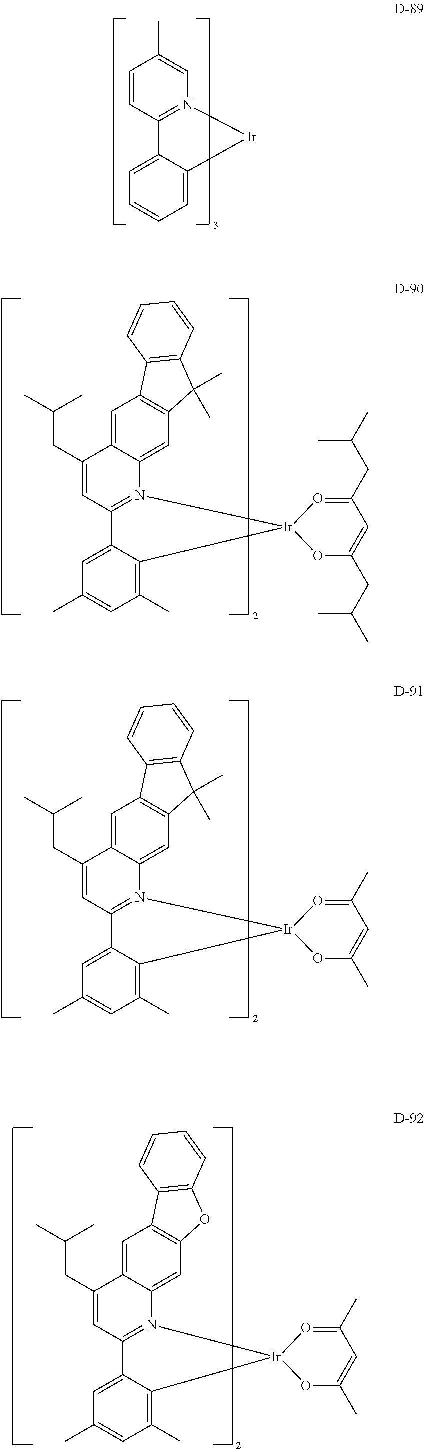

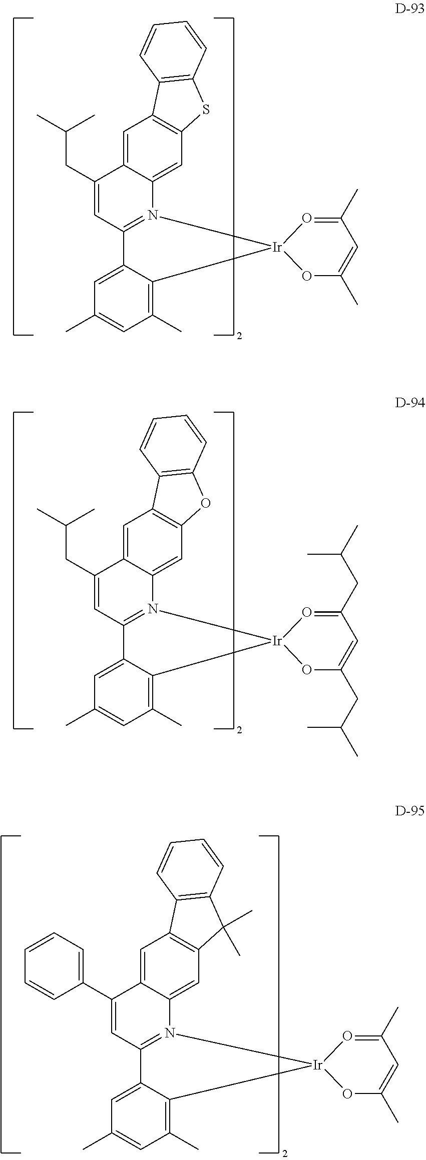

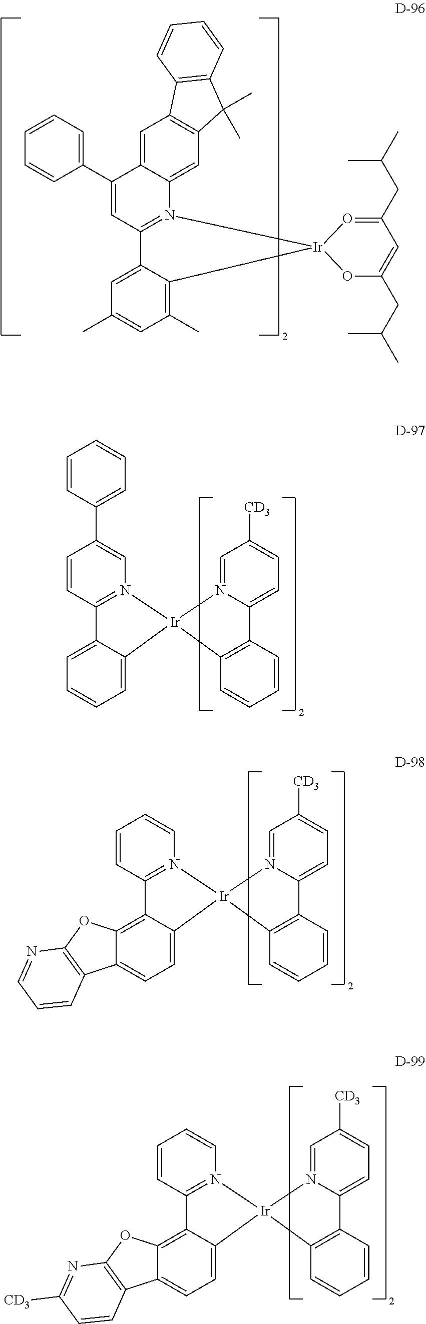

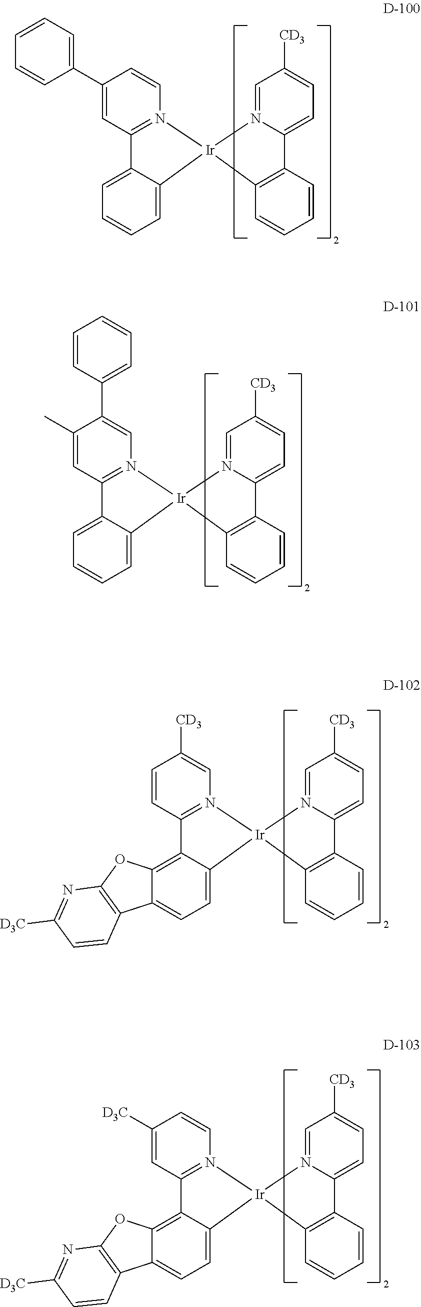

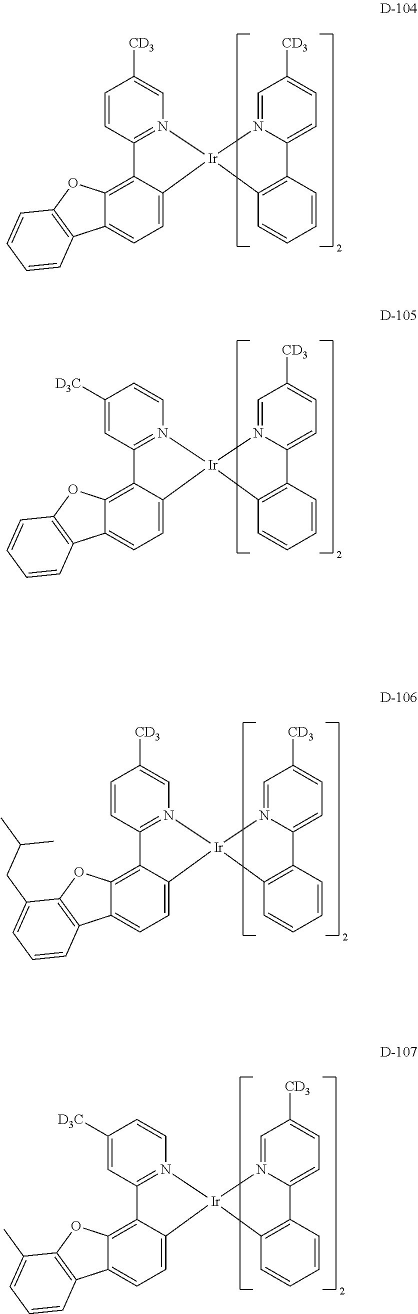

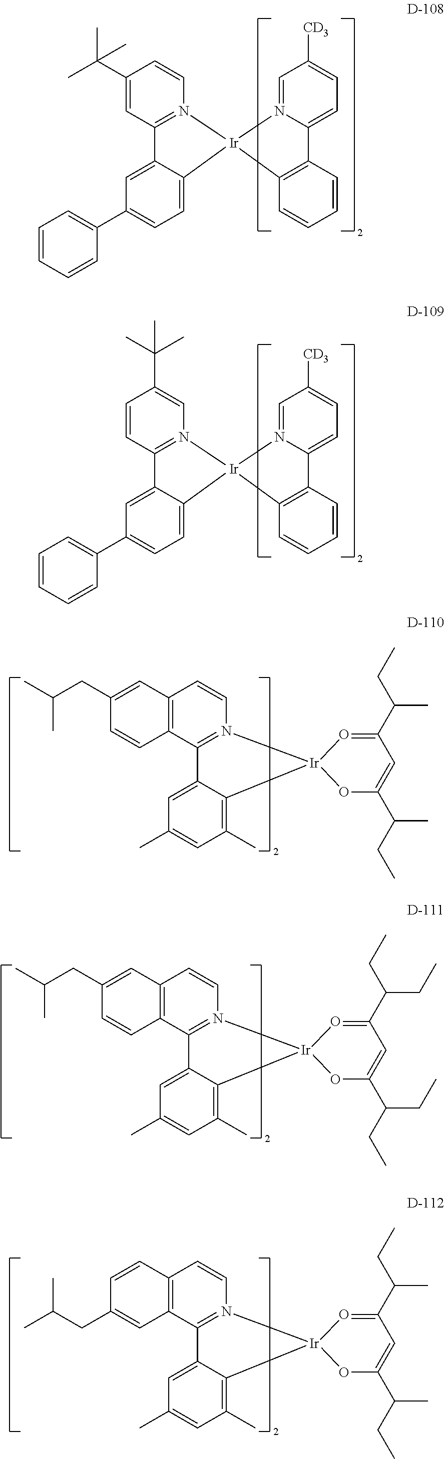

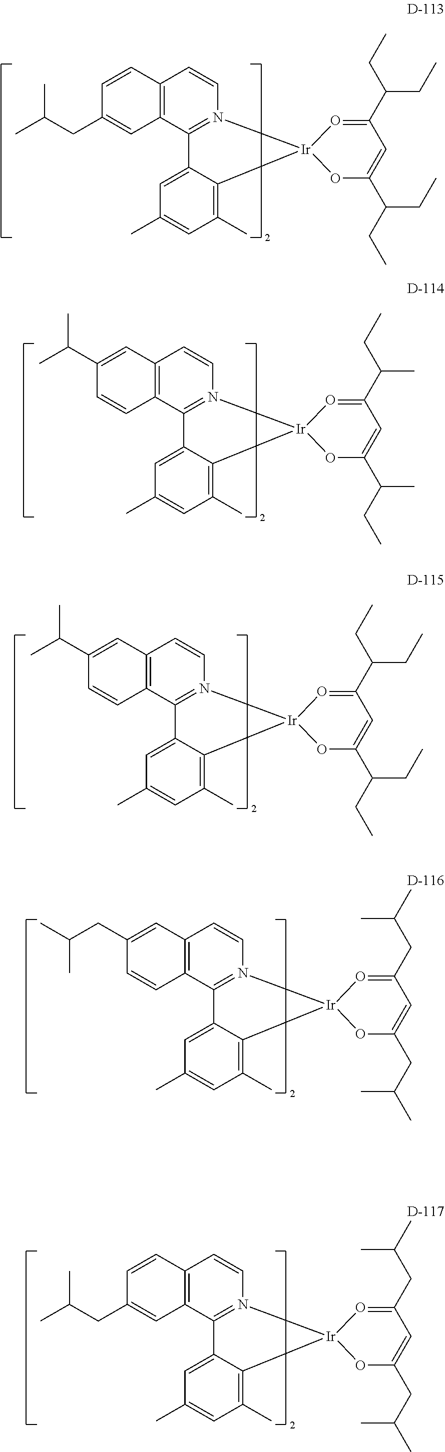

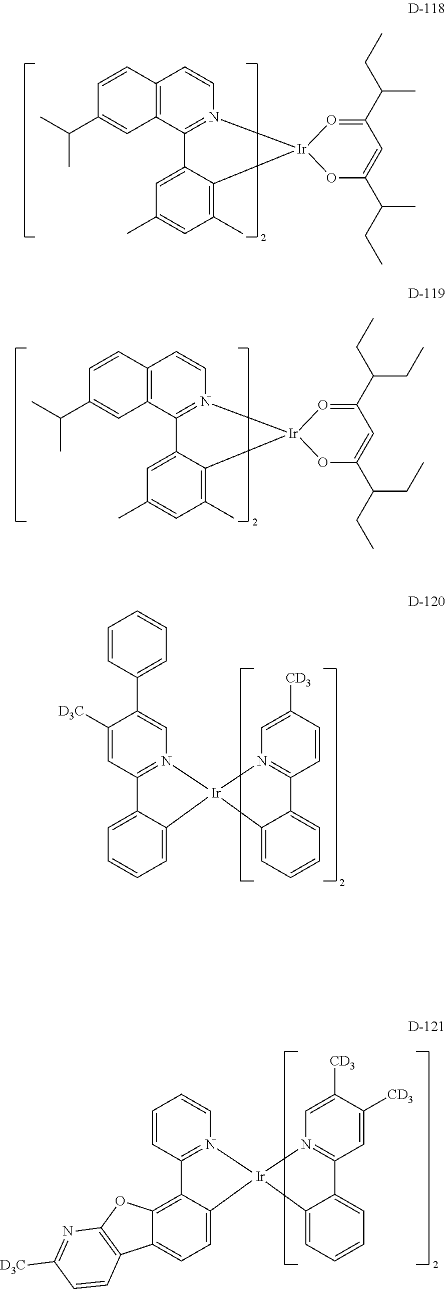

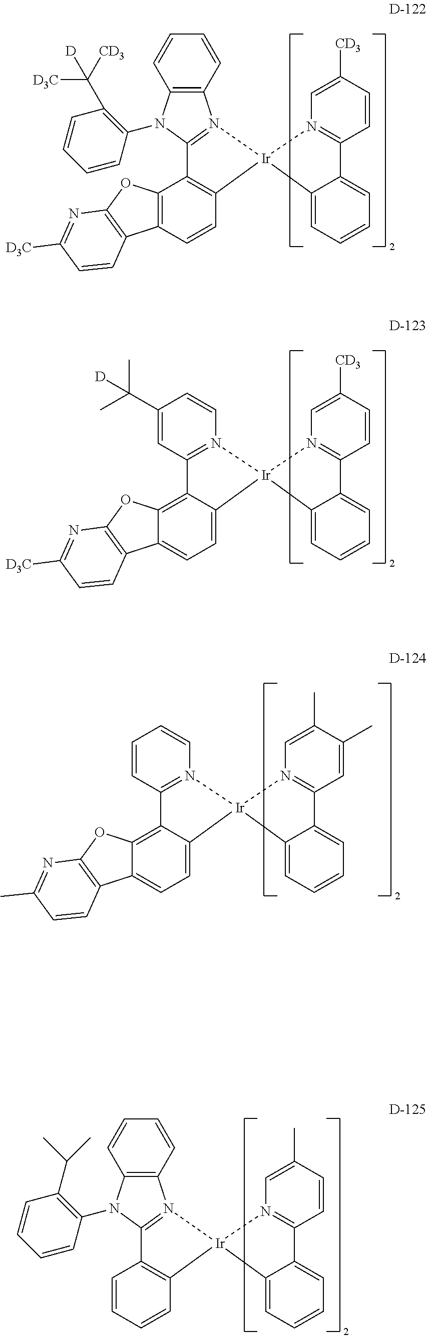

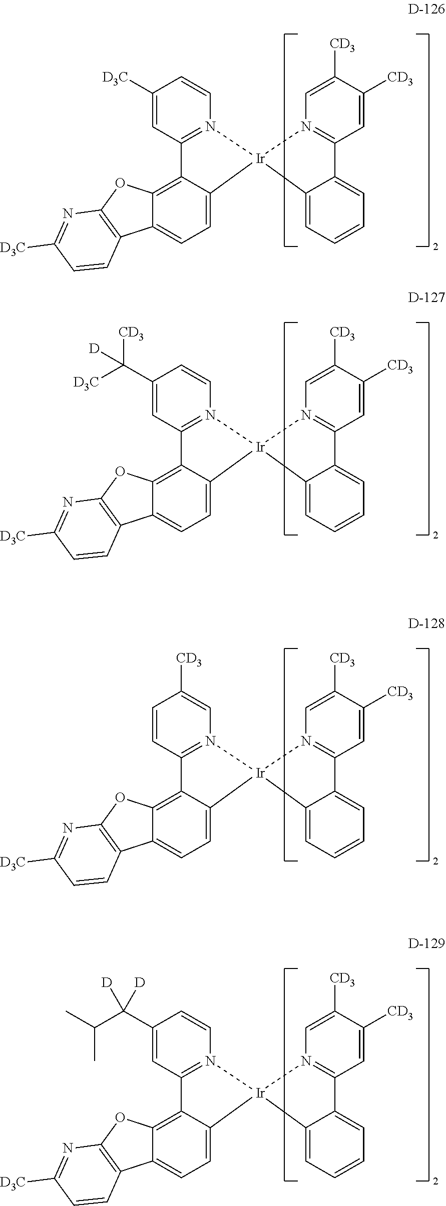

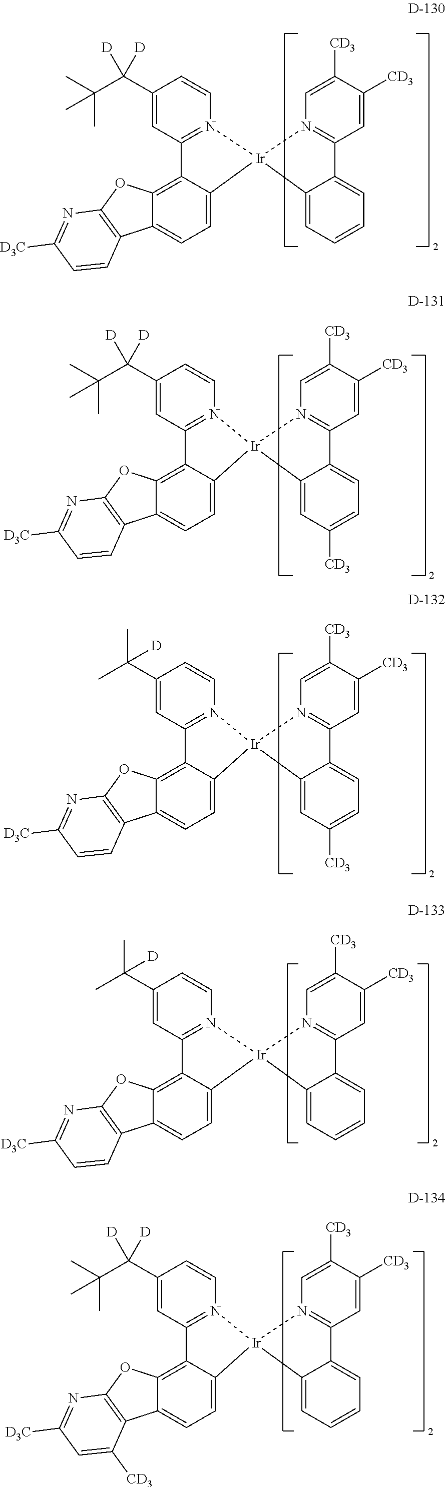

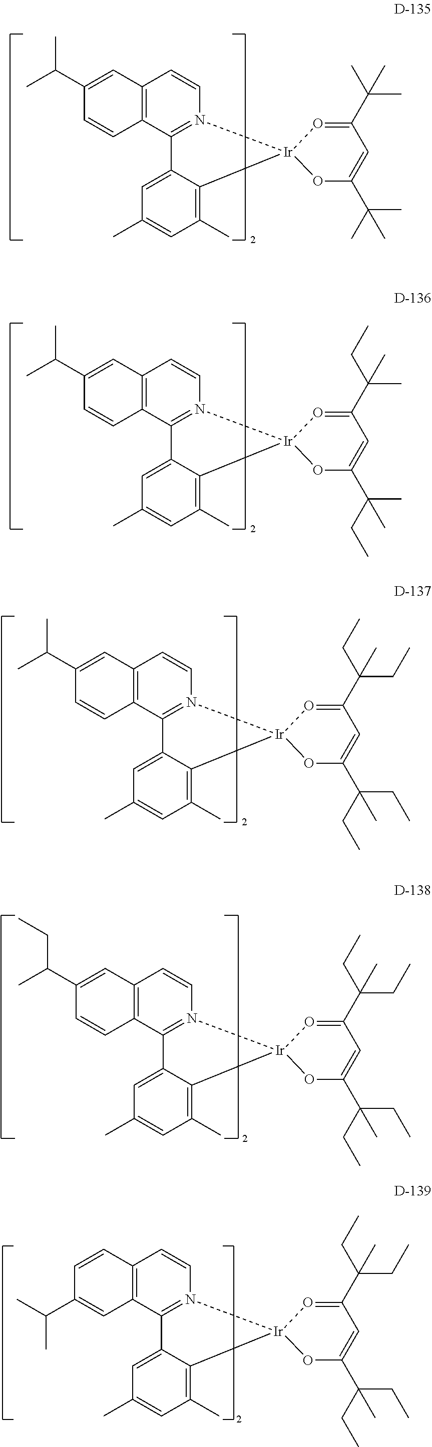

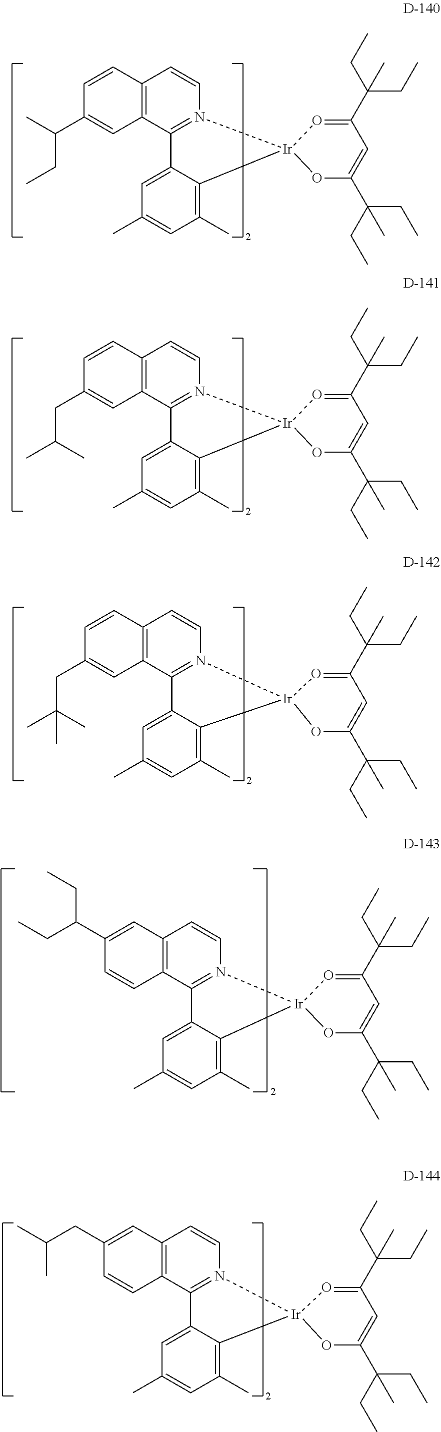

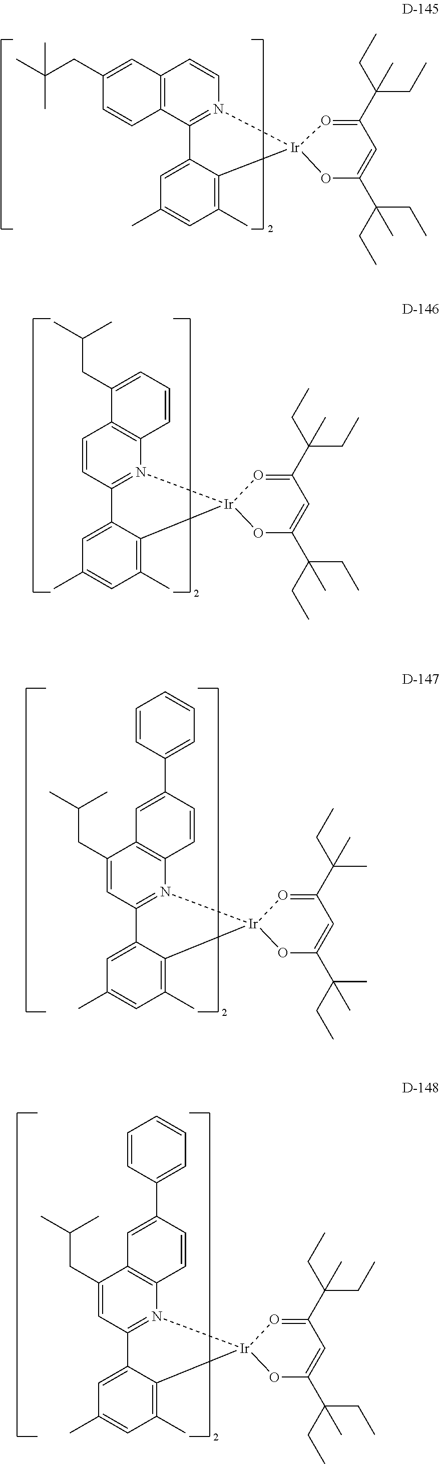

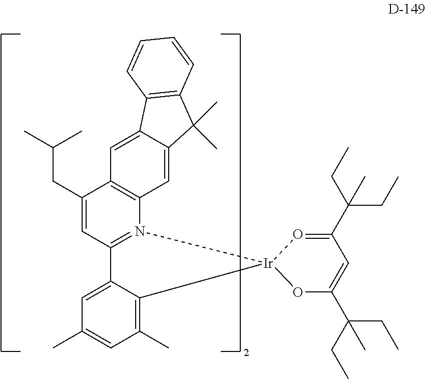

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used. When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

When forming a layer by the first host material and the second host material according to one embodiment, the layer can be formed by the above-listed methods, and can often be formed by co-deposition or mixed-deposition. The co-deposition is a mixed deposition method in which two or more materials are put into respective individual crucible sources and a current is applied to both cells simultaneously to evaporate the materials; and the mixed deposition is a method in which two or more materials are mixed in one crucible source before deposition, and then a current is applied to one cell to evaporate the materials.

According to one embodiment, when the first host material and the second host material exist in the same layer or different layers in the organic electroluminescent device, the layers by the two host compounds may be separately formed. For example, after depositing the first host material, a second host material may be deposited.

According to one embodiment, the present disclosure can provide display devices comprising an organic electroluminescent compound represented by the formula 1, and a plurality of host materials including a first host material represented by the formula 1 and a second host material represented by the formula 2 and/or the formula 3. In addition, by using the organic electroluminescent device of the present disclosure, display devices such as smartphones, tablets, notebooks, PCs, TVs, or display devices for vehicles, or lighting devices such as outdoor or indoor lighting can be prepared.

Hereinafter, the preparation method of compounds according to the present disclosure will be explained with reference to the synthesis method of a representative compound or intermediate compound in order to understand the present disclosure in detail.

### [Example 1] Synthesis of Compound C-277

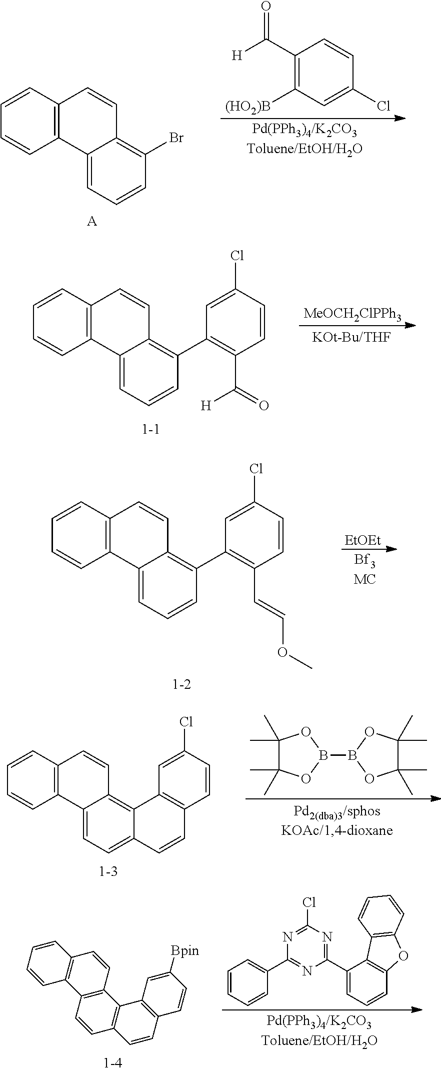

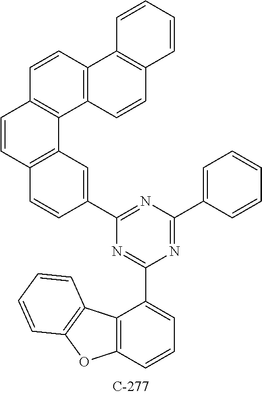 [SMILES: c1ccc(-c2nc(-c3ccc4ccc5ccc6c7ccccc7ccc6c5c4c3)nc(-c3cccc4oc5ccccc5c34)n2)cc1]

1) Synthesis of Compound 1-1

1-bromophenanthrene (50 g, 194 mmol), (5-chloro-2-formylphenyl)boronic acid (43.0 g, 233 mmol), Pd(PPh₃)₄ (11.2 g, 9.72 mmol), K₂CO₃ (67.2 g, 486 mmol), 600 mL of toluene, 300 mL of EtOH, and 450 mL of H₂O were added to a flask and dissolved. Thereafter, it was stirred under reflux at 140° C. for 3 hours. After completion of the reaction, the mixture was cooled to room temperature, and separated with a silica filter to obtain compound 1-1 (34.7 g, yield: 56%).

2) Synthesis of Compound 1-2

Compound 1-1 (34.6 g, 109.2 mmol) and (methoxymethyl)triphenylphosphonium chloride (MeOCH₂ClPPh₃) (56.2 g, 163.8 mmol) were added to 130 mL of THF, and then KOt-Bu (1M in THF) (163.8 mL, 163.8 mmol) was added dropwise thereto, followed by stirring at room temperature for 1.5 hours. After completion of the reaction, the organic layers were separated by adding ethyl acetate and water thereto followed by separating through a silica filter to obtain compound 1-2 (28.0 g, yield: 74.2%).

3) Synthesis of Compound 1-3

Compound 1-2 (16.0 g, 44.8 mmol) was dissolved in 400 mL of MC(Methyl chloride), and stirred at room temperature for one (1) day while adding dropwise BF₃EtOEt (31.0 mL, 247.1 mmol) thereto. After completion of the reaction, the organic layers were separated by adding MC and water thereto, followed by separating through a silica filter to obtain compound 1-3 (13.3 g, yield: 51.5%).

4) Synthesis of Compound 1-4

Compound 1-3 (4.0 g, 12.8 mmol), 4,4,4′,4′,5,5,5′,5′-octamethyl-2,2′-bi(1,3,2-dioxaborolane) (8.44 g, 33.2 mmol), Pd₂(dba)₃ (1.17 g, 1.28 mmol), sphos(0.525 g, 1.28 mmol), and KOAc (7.53 g, 76.7 mmol) were added to 120 mL of 1,4-dioxane, and stirred under reflux at 130° C. for 3 hours. Thereafter, it was separated by column chromatography to obtain compound 1-4 (5.00 g, yield: 96.7%).

5) Synthesis of Compound C-277

Compound 1-4 (4.0 g, 12.7 mmol), 2-chloro-4-(dibenzo[b,d]furan-1-yl)-6-phenyl-1,3,5-triazine (8.24 g, 32.5 mmol), Pd(PPh₃)₄ (0.844 g, 0.730 mmol), K₂CO₃ (5.05 g, 36.5 mmol), 40 mL of toluene, 15 mL of EtOH, and 15 mL of H₂O were added to a flask and dissolved. Thereafter, it was stirred under reflux at 140° C. for 3 hours. After completion of the reaction, the mixture was cooled to room temperature, and separated with a silica filter to obtain compound C-277 (5.7 g, yield: 77%).

| | MW | M.P | color |
|---|---|---|---|
| C-277 | 599.68 | 295.4° C. | yellow |

### [Example 2] Synthesis of Compound C-233

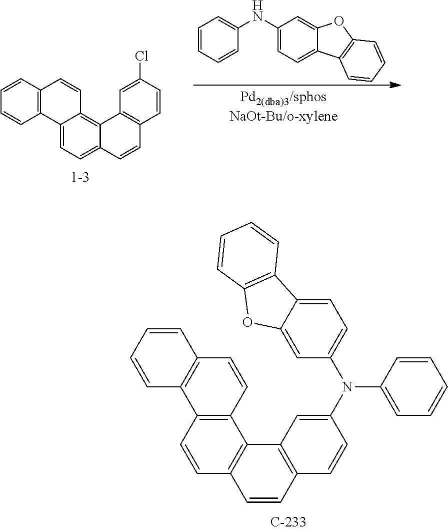 [SMILES: Clc1ccc2ccc3ccc4c5ccccc5ccc4c3c2c1 ; c1ccc(Nc2ccc3c(c2)oc2ccccc23)cc1 ; c1ccc(N(c2ccc3c(c2)oc2ccccc23)c2ccc3ccc4ccc5c6ccccc6ccc5c4c3c2)cc1]

Compound 1-3 (5.0 g, 16.0 mmol), N-phenyldibenzo[b,d]furan-3-amine (4.4 g, 16.8 mmol), Pd₂(dba)₃ (0.732 g, 0.800 mmol), Sphos (0.657 g, 1.60 mmol), NaOt-Bu (3.08 g, 32.0 mmol), and 80 mL of o-xylene were added to a flask and dissolved. Thereafter, it was stirred under reflux at 190° C. for 1 hour. After completion of the reaction, the mixture was cooled to room temperature and separated with a silica filter to obtain compound C-233 (5.0 g, yield: 58.4%).

| | MW | M.P | color |
|---|---|---|---|
| C-233 | 535.63 | 234.7° C. | Yellow |

### [Example 3] Synthesis of Compound C-282

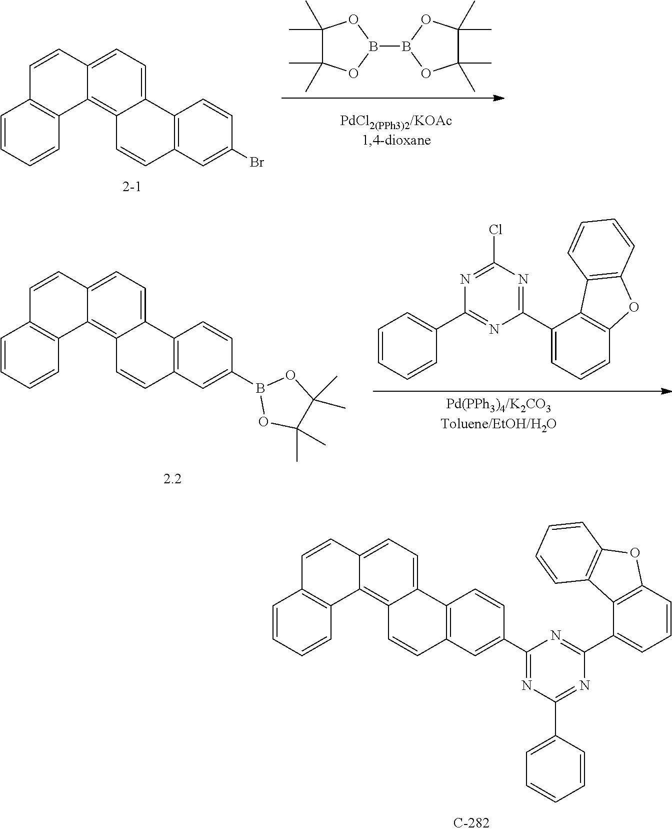

1) Synthesis of Compound 2-2

Compound 2-1 (15.0 g, 42.0 mmol), 4,4,4′,4′,5,5,5′,5′-octamethyl-2,2′-bi(1,3,2-dioxaborolane) (12.8 g, 50.4 mmol), PdCl₂(PPh₃)₂ (1.47 g, 2.10 mmol), KOAc (1.03 g, 10.5 mmol), and 210 mL of 1,4-dioxane were added to a flask and dissolved. Thereafter, it was stirred under reflux at 140° C. for 2 hours. After completion of the reaction, the mixture was cooled to room temperature and separated with a silica filter to obtain compound 2-2 (15.8 g, yield: 93.0%).

2) Synthesis of Compound C-282

Compound 2-2 (5.0 g, 12.4 mmol), 2-chloro-4-(dibenzo[b,d]furan-1-yl)-6-phenyl-1,3,5-triazine (4.0 g, 11.2 mmol), Pd(PPh₃)₄ (0.647 g, 0.56 mmol), K₂CO₃ (3.87 g, 28.0 mmol), 30 mL of toluene, 15 mL of EtOH, and 15 mL of H₂O were added to a flask and dissolved. Thereafter, it was stirred under reflux at 140° C. for 2 hours. After completion of the reaction, the mixture was cooled to room temperature and separated with a silica filter to obtain compound C-282 (3.2 g, yield: 47.6%).

| | MW | M.P | color |
|---|---|---|---|
| C-282 | 599.64 | 322.3° C. | Yellow |

### [Example 4] Synthesis of Compound C-343

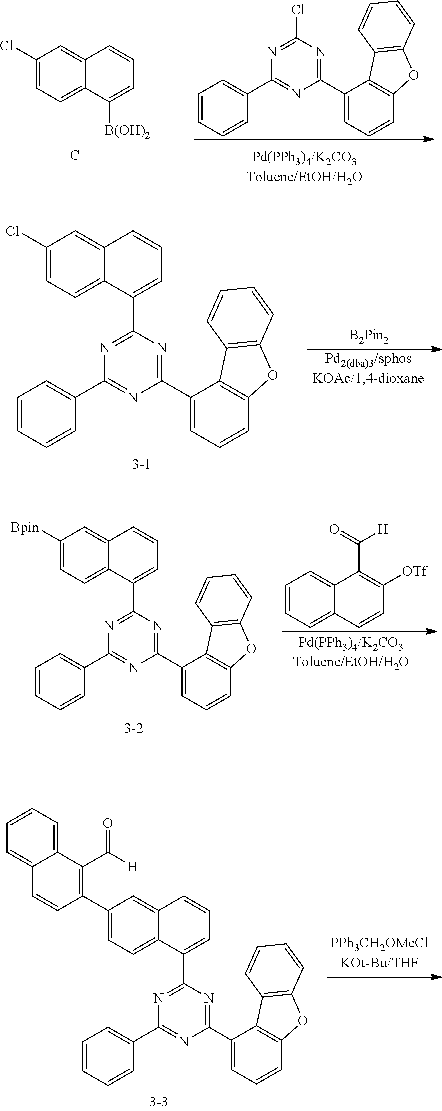

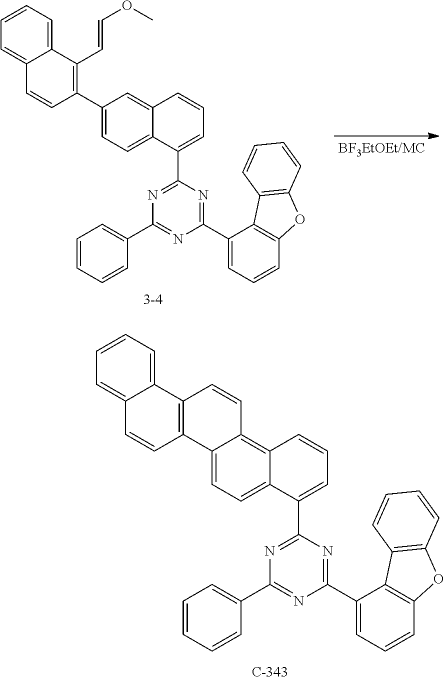 [SMILES: CO/C=C/c1c(-c2ccc3c(-c4nc(-c5ccccc5)nc(-c5cccc6oc7ccccc7c56)n4)cccc3c2)ccc2ccccc12 ; c1ccc(-c2nc(-c3cccc4c3ccc3c4ccc4c5ccccc5ccc43)nc(-c3cccc4oc5ccccc5c34)n2)cc1]

1) Synthesis of Compound 3-1

Compound C (17.4 g, 48.5 mmol), 2-chloro-4-(dibenzo[b,d]furan-1-yl)-6-phenyl-1,3,5-triazine (21.0 g, 72.8 mmol), Pd(PPh₃)₄ (1.68 g, 1.46 mmol), K₂CO₃ (20.1 g, 145.5 mmol), 200 mL of toluene, 50 mL of EtOH, and 50 mL of H₂O were added to a flask and dissolved. Then, it was stirred under reflux at 140° C. for 3 hours. After completion of the reaction, the mixture was cooled to room temperature and separated with a silica filter to obtain compound 3-1 (18.4 g, yield: 78%).

2) Synthesis of Compound 3-2

Compound 3-1 (18.4 g, 37.9 mmol), bis(pinacolato)diboron (B₂Pin₂) (13.5 g, 53.0 mmol), Pd₂(dba)₃ (1.73 g, 1.89 mmol), sphos(1.56 g, 3.79 mmol), and KOAc (11.1 g, 114 mmol) were added to 200 mL of 1,4-dioxane, and stirred at room temperature for 1.5 hours. After completion of the reaction, the organic layers were separated by adding ethyl acetate and water thereto, and then separated with a silica filter to obtain compound 3-2 (18.5 g, yield: 85%).

3) Synthesis of Compound 3-3

Compound 3-2 (13.2 g, 22.9 mmol) and 1-formylnaphthalene-2-yl trifluoromethanesulfonate (5.8 g, 19.1 mmol), Pd(PPh₃)₄ (0.661 g, 0.572 mmol), K₂CO₃ (7.90 g, 57.2 mmol), 50 mL of toluene, 25 mL of EtOH, and 25 mL of H₂O were added to a flask and dissolved. Then, it was stirred under reflux at 140° C. for 3 hours. After completion of the reaction, the mixture was cooled to room temperature and separated with a silica filter to obtain compound 3-3 (4.8 g, yield: 35%).

4) Synthesis of Compound 3-4

Compound 3-3 (4.0 g, 8.78 mmol) and (methoxymethyl)triphenylphosphonium chloride (PPh₃CH₂OMeCl) (3.40 g, 13.2 mmol) were added to 50 mL of THF, and then KOt-Bu (1M in THF) (9.93 mL, 13.2 mmol) was added dropwise thereto, followed by stirring at room temperature for 1.5 hours. After completion of the reaction, the organic layers were separated by adding ethyl acetate and water thereto, and then separated with a silica filter to obtain compound 3-4 (4.0 g, yield: 72%).

5) Synthesis of Compound C-343

Compound 3-4 (3.5 g, 5.54 mmol) and 400 mL of MC were added to a flask followed by dissolving. Thereafter, BF₃-EtOEt (0.894 mL, 16.6 mmol) was added dropwise thereto, and stirred at room temperature overnight. After completion of the reaction, the organic layers were separated by adding MC and water thereto, and then separated with a silica filter to obtain compound C-343 (13.3 g, yield: 51.5%).

| | MW | M.P | color |
|---|---|---|---|
| C-343 | 599.68 | 295.4° C. | yellow |

Hereinafter, the preparation method of an organic electroluminescent device comprising the organic electroluminescent compound of the present disclosure, and the property thereof will be explained in order to understand the present disclosure in detail.

### [Device Examples 1 to 4] Preparation of OLEDs According to the Present Disclosure which Emit Red Light

OLEDs according to the present disclosure were produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Then, compound HI-1 as a first hole injection compound was introduced into a cell of the vacuum vapor deposition apparatus, and compound HT-1 as a first hole transport compound was introduced into another cell. The two materials were evaporated at different rates and the first hole injection compound was deposited in a doping amount of 3 wt % based on the total amount of the first hole injection compound and the first hole transport compound to form a first hole injection layer having a thickness of 10 nm. Next, compound HT-1 was deposited as a first hole transport layer having a thickness of 80 nm on the first hole injection layer. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: The first host material and the second host material described in the following Table 1 were introduced into two cells of the vacuum vapor deposition apparatus as hosts, respectively, and compound D-39 was introduced into another cell as a dopant. The two host materials were evaporated at a rate of 1:1, and the dopant material was evaporated at a different rate, simultaneously and deposited in a doping amount of 3 wt % based on the total amount of the hosts and dopant to form a light-emitting layer having a thickness of 40 nm on the second hole transport layer. Next, compounds ETL-1 and EIL-1 as electron transport materials were deposited at a weight ratio of 50:50 to form an electron transport layer having a thickness of 35 nm on the light-emitting layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, OLEDs were produced. Each compound used for all the materials were purified by vacuum sublimation under 10⁻⁶ torr.

### [Comparative Examples 1 and 2] Preparation of OLEDs Comprising a Single Host Compound

OLEDs were manufactured in the same manner as in Device Example 1, except that the second host material of the following Table 1 alone was used as the host of the light-emitting layer.

The driving voltage, the luminous efficiency, and the light-emitting color at a luminance of 1,000 nits, and the time taken for luminance to decrease from 100% to 95% at a luminance of 5,000 nits (lifespan; T95) of the organic electroluminescent devices according to Device Examples 1 to 4 and Comparative Examples 1 and 2 produced as described above, were measured, and the results thereof are shown in Table 1 below:

TABLE 1

| | | | Driving | Luminous | Light- | |
|---|---|---|---|---|---|---|
| | First | Second | Voltage | Efficiency | Emitting | Lifespan |
| | host | host | (V) | (cd/A) | Color | (T95, hr) |
| Device | C-277 | H1-45 | 3.1 | 33.7 | Red | 492 |
| Example 1 | | | | | | |
| Device | C-282 | H1-45 | 3.1 | 32.5 | Red | 652 |
| Example 2 | | | | | | |
| Device | C-233 | C2-114 | 3.4 | 34.2 | Red | 437 |
| Example 3 | | | | | | |
| Device | C-343 | H1-129 | 3.1 | 34.3 | Red | 302 |
| Example 4 | | | | | | |
| Comparative | — | CBP | 9.0 | 14.3 | Red | 0.31 |
| Example 1 | | | | | | |
| Comparative | — | C2-114 | 3.6 | 29.4 | Red | 31.6 |
| Example 2 | | | | | | |

### [Device Example 5 to 7] Preparation of OLEDs According to the Present Disclosure which Emit Blue Light

OLEDs according to the present disclosure were produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropanol and then used. Thereafter, the ITO substrate was mounted on a substrate holder of a vacuum vapor deposition apparatus. Then, compound HI-1 as a first hole injection compound was introduced into a cell of the vacuum vapor deposition apparatus, and compound HT-3 was introduced into another cell. The two materials were evaporated at different rates and compound HI-1 was deposited in a doping amount of 3 wt % based on the total amount of compounds HI-1 and HI-3 to form a hole injection layer having a thickness of 10 nm. Next, compound HT-3 was deposited as a first hole transport layer having a thickness of 80 nm on the hole injection layer. Next, compound HT-4 was then introduced into another cell of the vacuum vapor deposition apparatus and was evaporated by applying an electric current to the cell, thereby forming a second hole transport layer having a thickness of 5 nm on the first hole transport layer. After forming the hole injection layer and the hole transport layers, a light-emitting layer was formed thereon as follows: Compound BH was introduced into one of the two cells of the vacuum deposition apparatus as hosts, and compound BD was introduced into another cell as a dopant. The dopant material was evaporated at a different rate, simultaneously, was deposited in a doping amount of 3 wt % based on the total amount of the host and the dopant to form a light-emitting layer having a thickness of 20 nm on the second hole transport layer. Next, the compound described in the following Table 2 as an electron buffer layer was deposited on the light-emitting layer to form an electron buffer layer having a thickness of 5 nm. Next, compounds ETL-1 and EIL-1 as electron transport materials were deposited at a weight ratio of 50:50 to form an electron transport layer having a thickness of 30 nm on the electron buffer layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, OLEDs were produced. Each compound used for all the materials were purified by vacuum sublimation under 10⁻⁶ torr.

### [Comparative Example 3] Preparation of OLED Comprising a Comparative Compound as an Electron Buffer Layer

An OLED was manufactured in the same manner as in Device Example 5, except that the compound described in the following Table 2 was used as an electron buffer layer.

The driving voltage, the luminous efficiency, and the light-emitting color at a luminance of 1,000 nits, and the time taken for luminance to decrease from 100% to 95% at a luminance of 2,000 nits (lifespan; T95) of the OLEDs according to Device Examples 5 to 7 and Comparative Example 3 produced as described above, were measured, and the results thereof are shown in Table 2 below:

TABLE 2

| | | Driving | Luminous | Light- | |
|---|---|---|---|---|---|
| | Electron Buffer | Voltage | Efficiency | Emitting | Lifespan |
| | Layer | (V) | (cd/A) | Color | T95(hr) |
| Comparative | T-1 | 4.8 | 4.9 | Blue | 29 |
| Example 3 | | | | | |
| Device | C-282 | 4.3 | 5.3 | Blue | 66 |
| Example 5 | | | | | |
| Device | C-277 | 3.8 | 6.3 | Blue | 56 |
| Example 6 | | | | | |
| Device | C-343 | 4.2 | 5.4 | Blue | 69 |
| Example 7 | | | | | |

The compounds used in Device Examples and Comparative Examples above are specifically shown in the following Table 3.

TABLE 3

| | |
|---|---|
| Hole Injection Layer/ Hole Transport Layer | 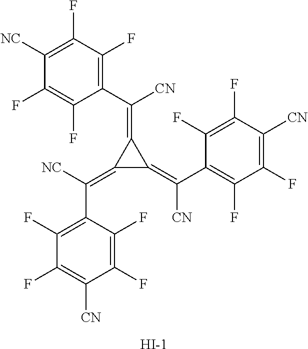 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 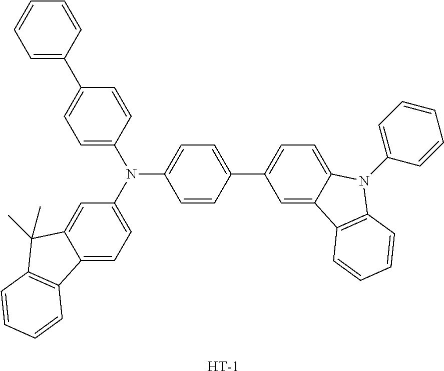 |
| | 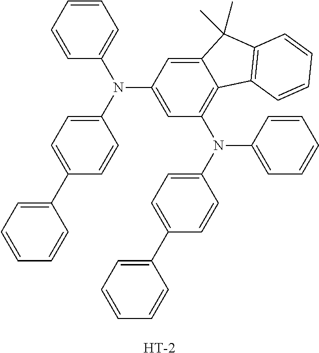 |
| | 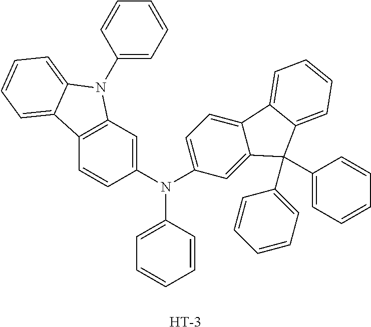 |
| | 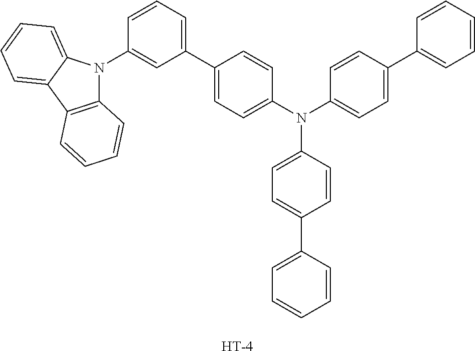 |
| Light-Emitting Layer/ Electron Buffer Layer | 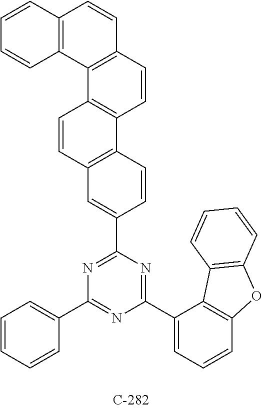 [SMILES: c1ccc(-c2nc(-c3ccc4c(ccc5c4ccc4ccc6ccccc6c45)c3)nc(-c3cccc4oc5ccccc5c34)n2)cc1] |
| | 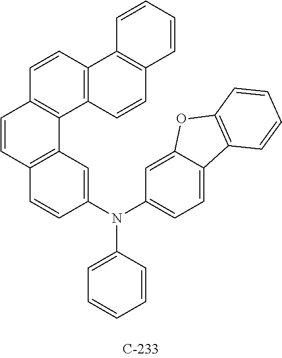 [SMILES: c1ccc(N(c2ccc3c(c2)oc2ccccc23)c2ccc3ccc4ccc5c6ccccc6ccc5c4c3c2)cc1] |
| | 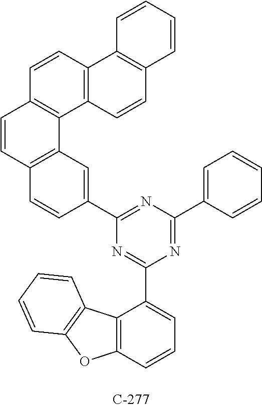 [SMILES: c1ccc(-c2nc(-c3ccc4ccc5ccc6c7ccccc7ccc6c5c4c3)nc(-c3cccc4oc5ccccc5c34)n2)cc1] |
| | 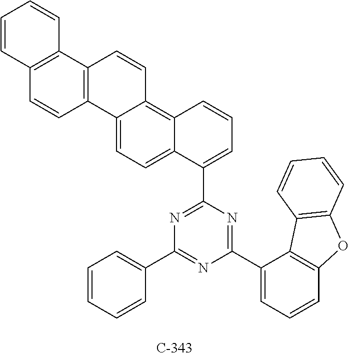 [SMILES: c1ccc(-c2nc(-c3cccc4c3ccc3c4ccc4c5ccccc5ccc43)nc(-c3cccc4oc5ccccc5c34)n2)cc1] |
| | 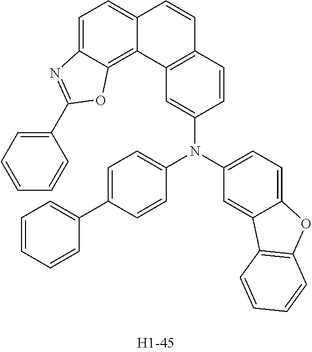 [SMILES: c1ccc(-c2ccc(N(c3ccc4oc5ccccc5c4c3)c3ccc4ccc5ccc6nc(-c7ccccc7)oc6c5c4c3)cc2)cc1] |
| | 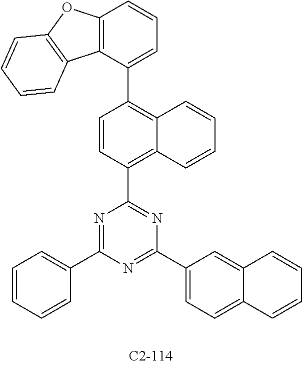 [SMILES: c1ccc(-c2nc(-c3ccc4ccccc4c3)nc(-c3ccc(-c4cccc5oc6ccccc6c45)c4ccccc34)n2)cc1] |
| | 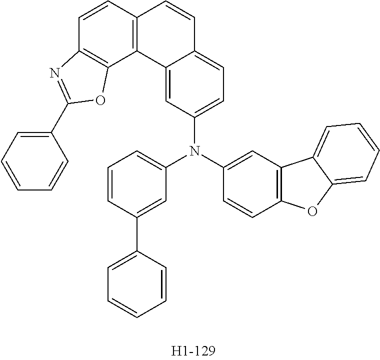 [SMILES: c1ccc(-c2cccc(N(c3ccc4oc5ccccc5c4c3)c3ccc4ccc5ccc6nc(-c7ccccc7)oc6c5c4c3)c2)cc1] |
| | 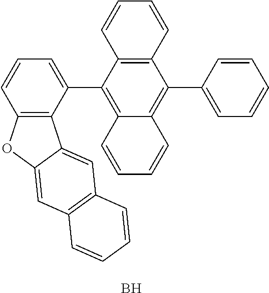 |
| | 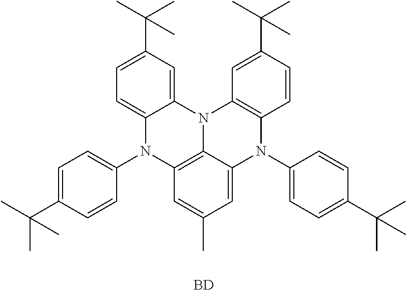 |
| | 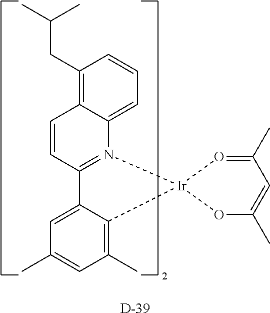 |
| | 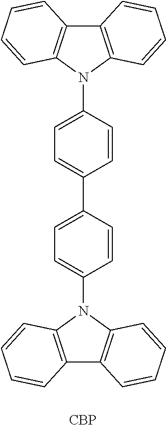 |
| Electron Transport Layer/Electron Injection Layer | 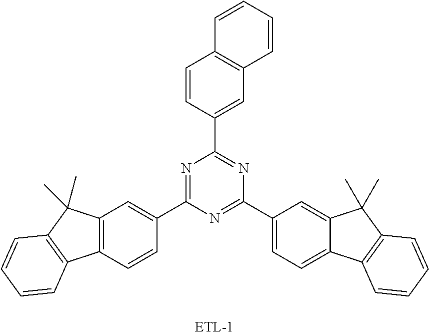 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 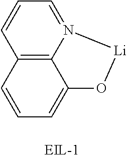 |

1. An organic electroluminescent compound represented by the following formula 1: 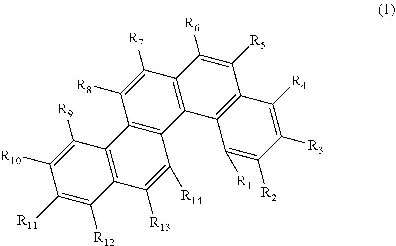
Wherein
R₁ to R₁₄ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of a (C3-C30) aliphatic ring and a (C6-C30) aromatic ring, -(L₁)ₙ-(HAr)ₘ, or -L₃-N—(Ar₃)(Ar₄); or may be linked to the adjacent substituent each other to form a ring(s);
provided that at least one of R₁ to R₁₄ is -(L₁)ₙ-(HAr)ₘ or -L₃-N—(Ar₃)(Ar₄);
L₁ represents single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
HAr represents a substituted or unsubstituted (C6-C30)aryl or a substituted or unsubstituted (3- to 30-membered)heteroaryl;
L₃ represents a single bond, a substituted or unsubstituted (C6-C30)arylene, or a substituted or unsubstituted (3- to 30-membered)heteroarylene;
Ar₃ and Ar₄ each independently represent hydrogen, a substituted or unsubstituted (C1-C30)alkyl, a substituted or unsubstituted (C2-C30)alkenyl, a substituted or unsubstituted fused ring of a (C3-C30) aliphatic ring and a (C6-C30) aromatic ring, a substituted or unsubstituted (C6-C30)aryl, or a substituted or unsubstituted (3- to 30-membered)heteroaryl; and
n and m each independently represent an integer of 1 or 2; and when n and m are an integer of 2, each of L₁ and each of HAr may be the same or different;
provided that the following compounds are excluded from the organic electroluminescent compound represented by the formula 1.

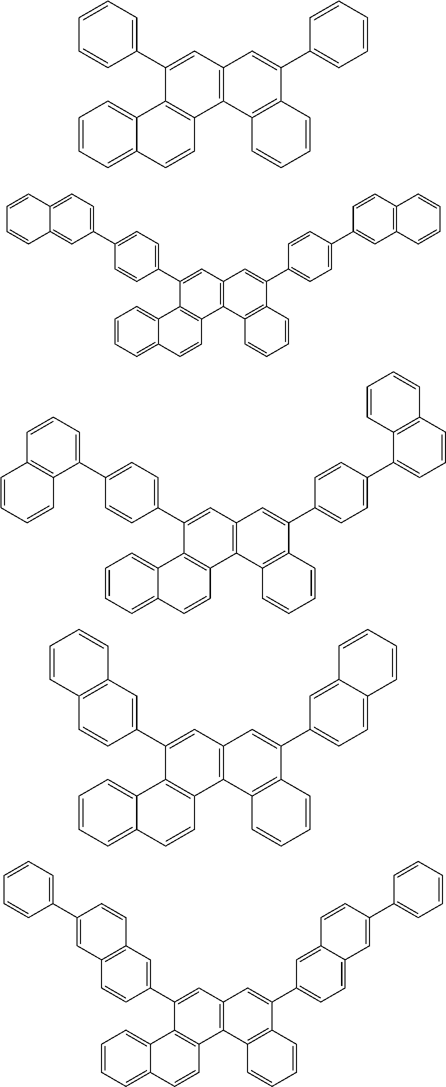

2. The organic electroluminescent compound according to claim 1, wherein at least one of R₁ to R₁₄ is -(L₁)ₙ-(HAr)ₘ.

3. The organic electroluminescent compound according to claim 1, wherein at least one of R₁ to R₁₄ is -L₃-N—(Ar₃)(Ar₄).

4. The organic electroluminescent compound according to claim 1, wherein the substituent(s) of the substituted (C1-C30)alkyl, the substituted (C2-C30)alkenyl, the substituted (C6-C30)aryl(ene), the substituted (3- to 30-membered)heteroaryl(ene), the substituted (C3-C30)cycloalkyl, the substituted (C1-C30)alkoxy, the substituted fused ring of (C3-C30) aliphatic ring and (C6-C30) aromatic ring, the substituted tri(C1-C30)alkylsilyl, the substituted di(C1-C30)alkyl(C6-C30)arylsilyl, the substituted (C1-C30)alkyldi(C6-C30)arylsilyl, and the substituted tri(C6-C30)arylsilyl each independently represents at least one selected from the group consisting of deuterium; halogen; cyano; carboxyl; nitro; hydroxy; (C1-C30)alkyl; halo(C1-C30)alkyl; (C2-C30)alkenyl; (C2-C30)alkynyl; (C1-C30)alkoxy; (C1-C30)alkylthio; (C3-C30)cycloalkyl; (C3-C30)cycloalkenyl; (3- to 7-membered)heterocycloalkyl; (C6-C30)aryloxy; (C6-C30)arylthio; (3- to 30-membered)heteroaryl unsubstituted or substituted with at least one (C6-C30)aryl; (C6-C30)aryl unsubstituted or substituted with at least one (C1-C30)alkyl and (3- to 30-membered)heteroaryl; tri(C1-C30)alkylsilyl; tri(C6-C30)arylsilyl; di(C1-C30)alkyl(C6-C30)arylsilyl; (C1-C30)alkyldi(C6-C30)arylsilyl; amino; mono- or di-(C1-C30)alkylamino; mono- or di-(C2-C30)alkenylamino; mono- or di-(C6-C30)arylamino unsubstituted or substituted with (C1-C30)alkyl; mono- or di-(3- to 30-membered)heteroarylamino; (C1-C30)alkyl(C2-C30)alkenylamino; (C1-C30)alkyl(C6-C30)arylamino; (C1-C30)alkyl(3- to 30-membered)heteroarylamino; (C2-C30)alkenyl(C6-C30)arylamino; (C2-C30)alkenyl(3- to 30-membered)heteroarylamino; (C6-C30)aryl(3- to 30-membered)heteroarylamino; (C1-C30)alkylcarbonyl; (C1-C30)alkoxycarbonyl; (C6-C30)arylcarbonyl; (C6-C30)arylphosphinyl, di(C6-C30)arylboronyl; di(C1-C30)alkylboronyl; (C1-C30)alkyl(C6-C30)arylboronyl; (C6-C30)ar(C1-C30)alkyl; and (C1-C30)alkyl(C6-C30)aryl.

5. The organic electroluminescent compound according to claim 1, wherein the compound represented by the formula 1 is selected from the following compounds:

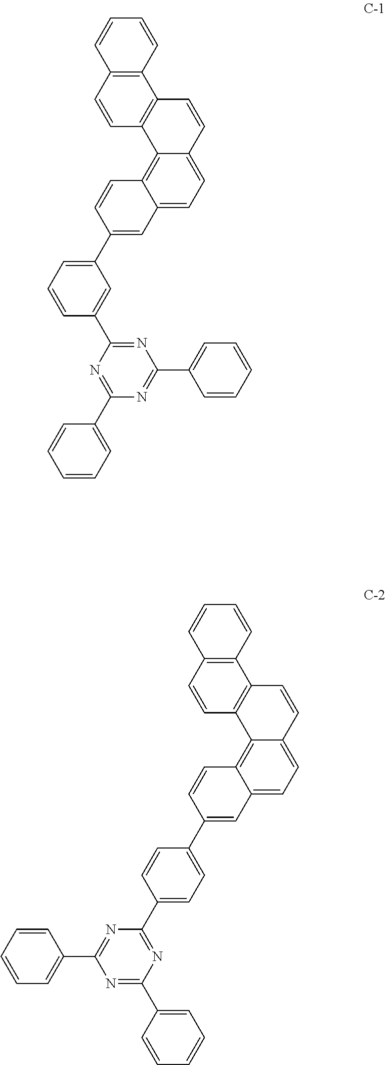 [SMILES: c1ccc(-c2nc(-c3ccccc3)nc(-c3cccc(-c4ccc5c(ccc6ccc7c8ccccc8ccc7c65)c4)c3)n2)cc1 ; c1ccc(-c2nc(-c3ccccc3)nc(-c3ccc(-c4ccc5c(ccc6ccc7c8ccccc8ccc7c65)c4)cc3)n2)cc1]

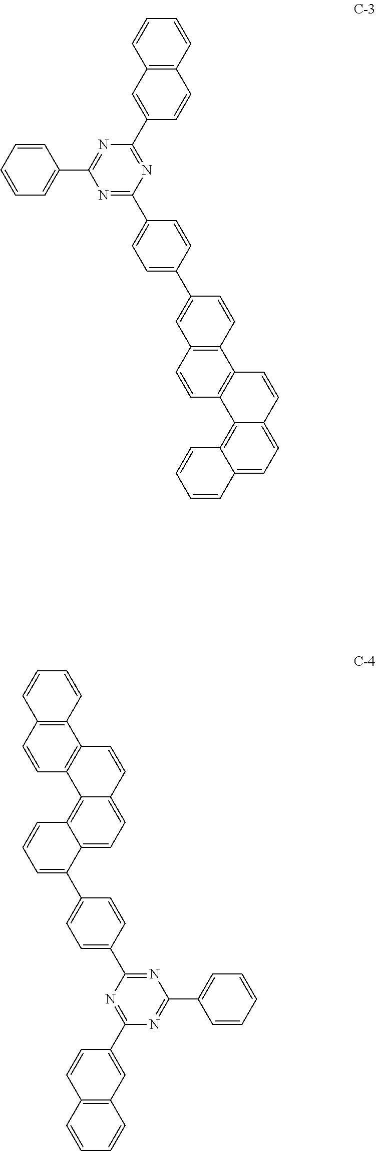 [SMILES: c1ccc(-c2nc(-c3ccc(-c4ccc5c(ccc6c5ccc5ccc7ccccc7c56)c4)cc3)nc(-c3ccc4ccccc4c3)n2)cc1 ; c1ccc(-c2nc(-c3ccc(-c4cccc5c4ccc4ccc6c7ccccc7ccc6c45)cc3)nc(-c3ccc4ccccc4c3)n2)cc1]

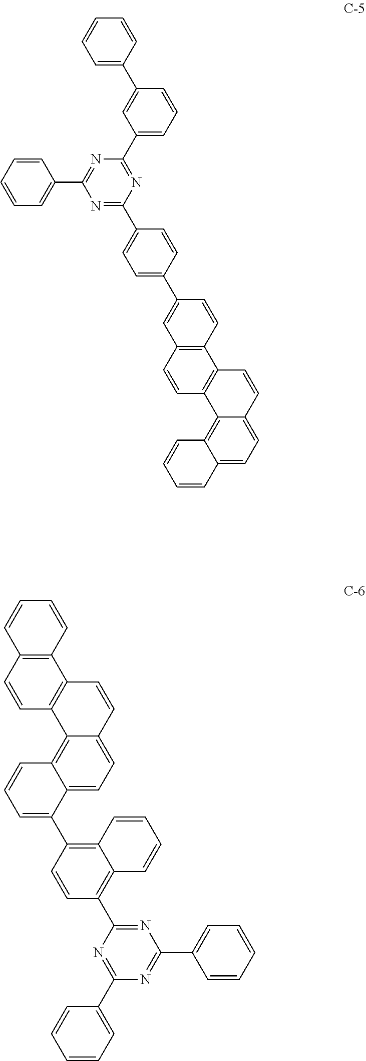 [SMILES: c1ccc(-c2cccc(-c3nc(-c4ccccc4)nc(-c4ccc(-c5ccc6c(ccc7c6ccc6ccc8ccccc8c67)c5)cc4)n3)c2)cc1 ; c1ccc(-c2nc(-c3ccccc3)nc(-c3ccc(-c4cccc5c4ccc4ccc6c7ccccc7ccc6c45)c4ccccc34)n2)cc1]

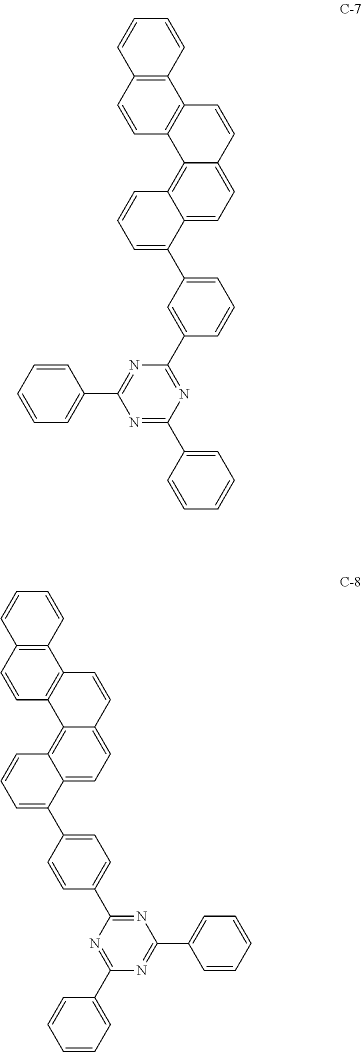 [SMILES: c1ccc(-c2nc(-c3ccccc3)nc(-c3cccc(-c4cccc5c4ccc4ccc6c7ccccc7ccc6c45)c3)n2)cc1 ; c1ccc(-c2nc(-c3ccccc3)nc(-c3ccc(-c4cccc5c4ccc4ccc6c7ccccc7ccc6c45)cc3)n2)cc1]

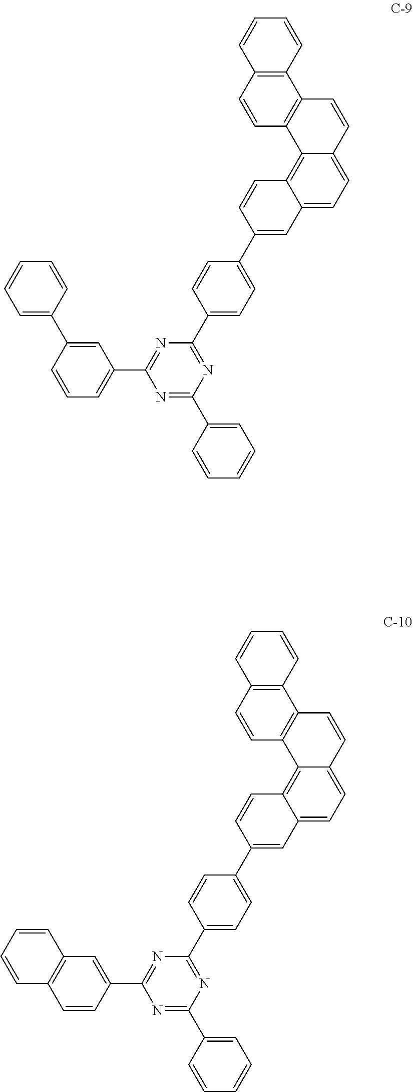 [SMILES: c1ccc(-c2cccc(-c3nc(-c4ccccc4)nc(-c4ccc(-c5ccc6c(ccc7ccc8c9ccccc9ccc8c76)c5)cc4)n3)c2)cc1 ; c1ccc(-c2nc(-c3ccc(-c4ccc5c(ccc6ccc7c8ccccc8ccc7c65)c4)cc3)nc(-c3ccc4ccccc4c3)n2)cc1]

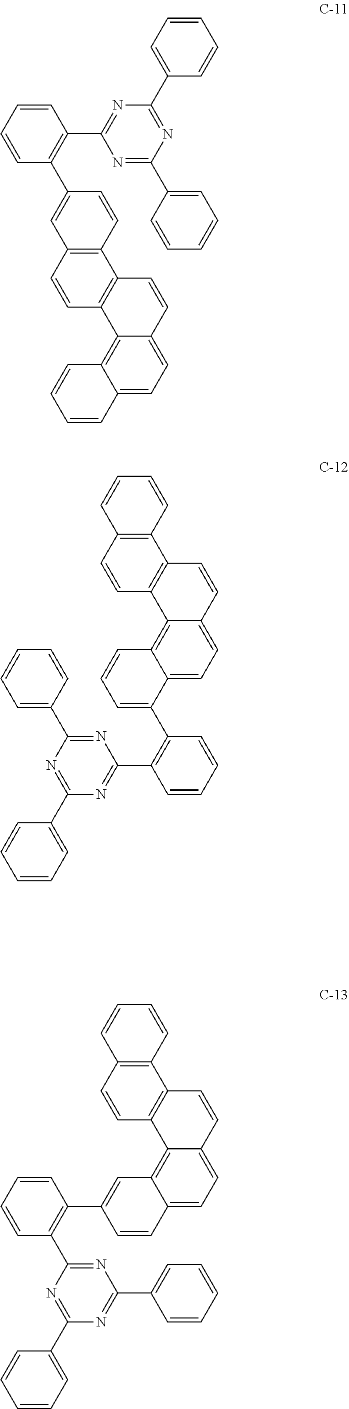 [SMILES: c1ccc(-c2nc(-c3ccccc3)nc(-c3ccccc3-c3ccc4c(ccc5c4ccc4ccc6ccccc6c45)c3)n2)cc1 ; c1ccc(-c2nc(-c3ccccc3)nc(-c3ccccc3-c3cccc4c3ccc3ccc5c6ccccc6ccc5c34)n2)cc1 ; c1ccc(-c2nc(-c3ccccc3)nc(-c3ccccc3-c3ccc4ccc5ccc6c7ccccc7ccc6c5c4c3)n2)cc1]

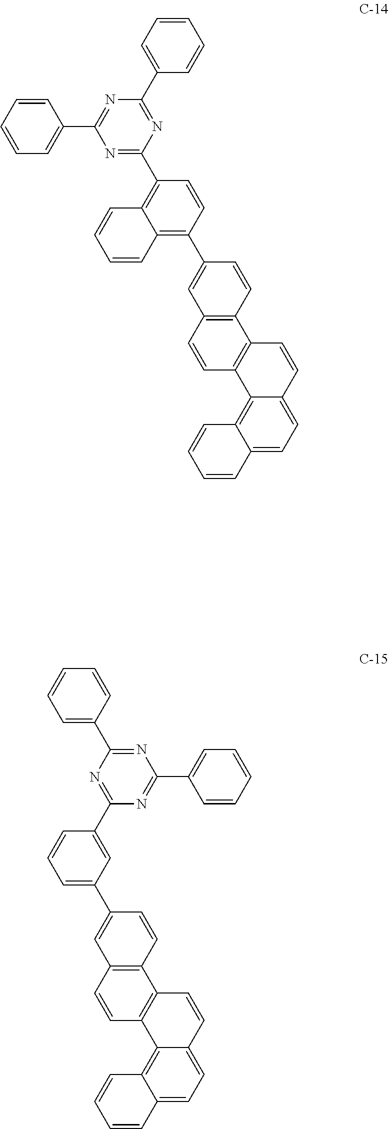 [SMILES: c1ccc(-c2nc(-c3ccccc3)nc(-c3ccc(-c4ccc5c(ccc6c5ccc5ccc7ccccc7c56)c4)c4ccccc34)n2)cc1 ; c1ccc(-c2nc(-c3ccccc3)nc(-c3cccc(-c4ccc5c(ccc6c5ccc5ccc7ccccc7c56)c4)c3)n2)cc1]

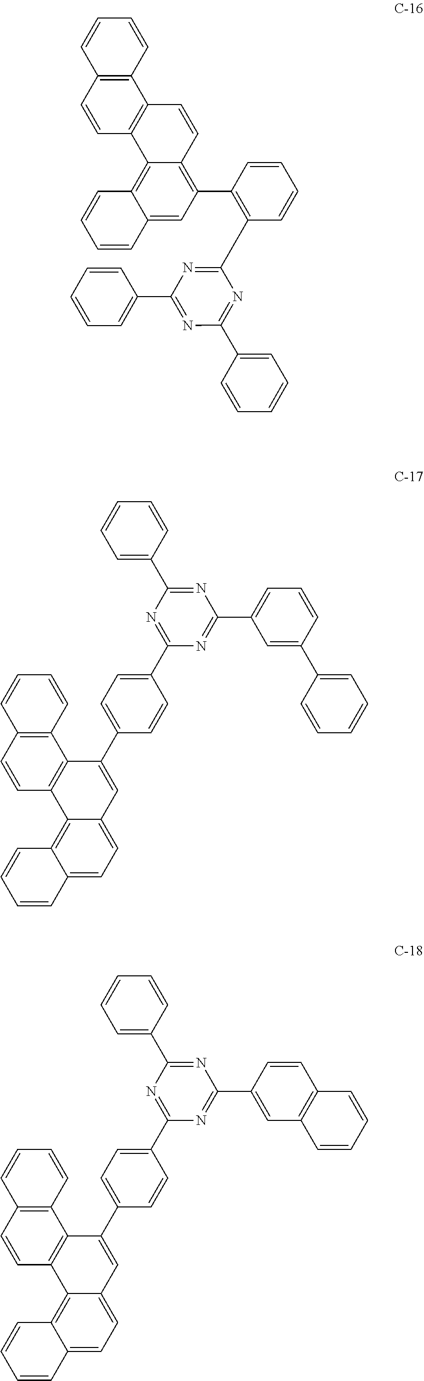 [SMILES: c1ccc(-c2nc(-c3ccccc3)nc(-c3ccccc3-c3cc4ccccc4c4c3ccc3c5ccccc5ccc34)n2)cc1 ; c1ccc(-c2cccc(-c3nc(-c4ccccc4)nc(-c4ccc(-c5cc6ccc7ccccc7c6c6ccc7ccccc7c56)cc4)n3)c2)cc1 ; c1ccc(-c2nc(-c3ccc(-c4cc5ccc6ccccc6c5c5ccc6ccccc6c45)cc3)nc(-c3ccc4ccccc4c3)n2)cc1]

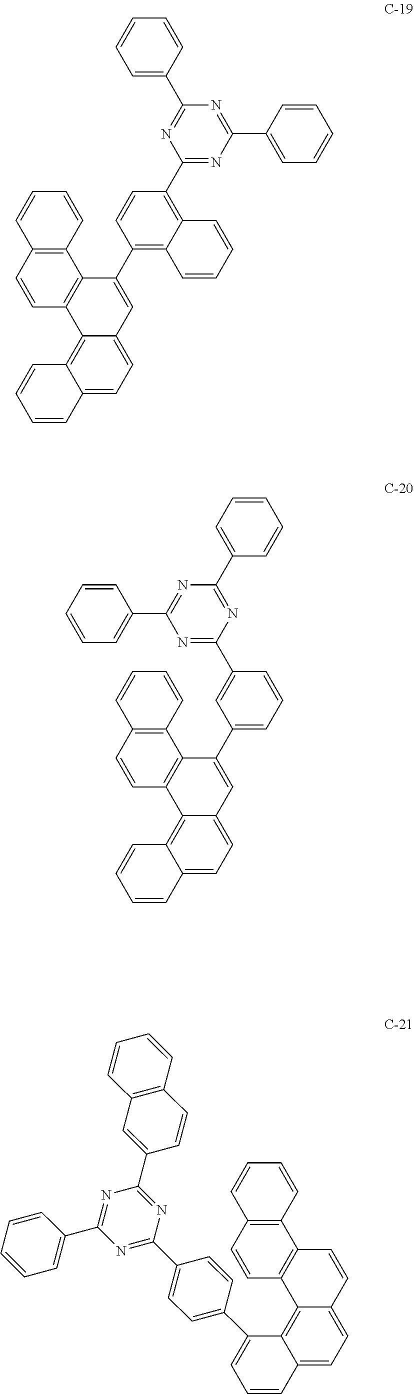 [SMILES: c1ccc(-c2nc(-c3ccccc3)nc(-c3ccc(-c4cc5ccc6ccccc6c5c5ccc6ccccc6c45)c4ccccc34)n2)cc1 ; c1ccc(-c2nc(-c3ccccc3)nc(-c3cccc(-c4cc5ccc6ccccc6c5c5ccc6ccccc6c45)c3)n2)cc1 ; c1ccc(-c2nc(-c3ccc(-c4cccc5ccc6ccc7c8ccccc8ccc7c6c45)cc3)nc(-c3ccc4ccccc4c3)n2)cc1]

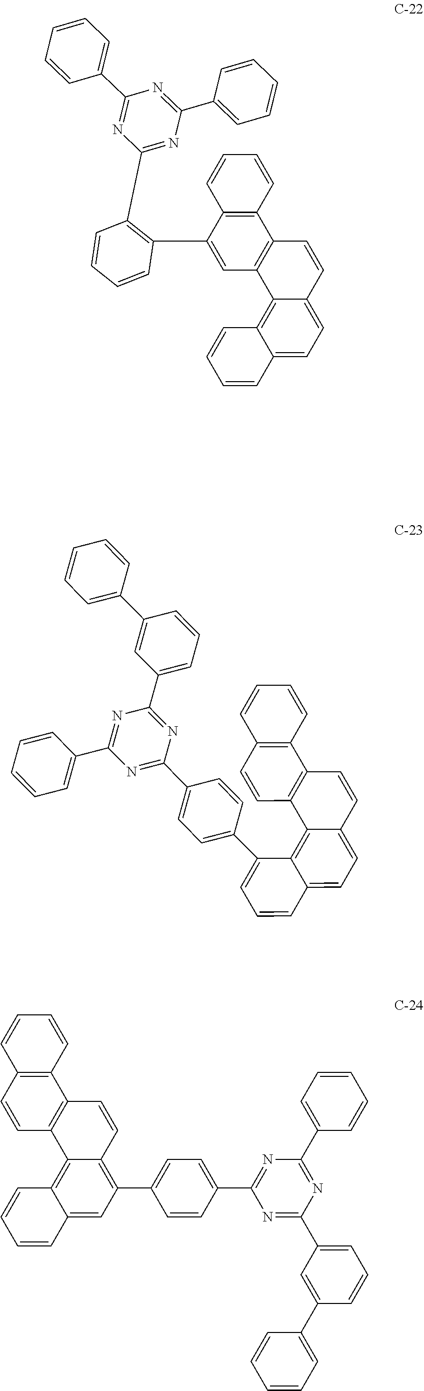 [SMILES: c1ccc(-c2nc(-c3ccccc3)nc(-c3ccccc3-c3cc4c(ccc5ccc6ccccc6c54)c4ccccc34)n2)cc1 ; c1ccc(-c2cccc(-c3nc(-c4ccccc4)nc(-c4ccc(-c5cccc6ccc7ccc8c9ccccc9ccc8c7c56)cc4)n3)c2)cc1 ; c1ccc(-c2cccc(-c3nc(-c4ccccc4)nc(-c4ccc(-c5cc6ccccc6c6c5ccc5c7ccccc7ccc56)cc4)n3)c2)cc1]

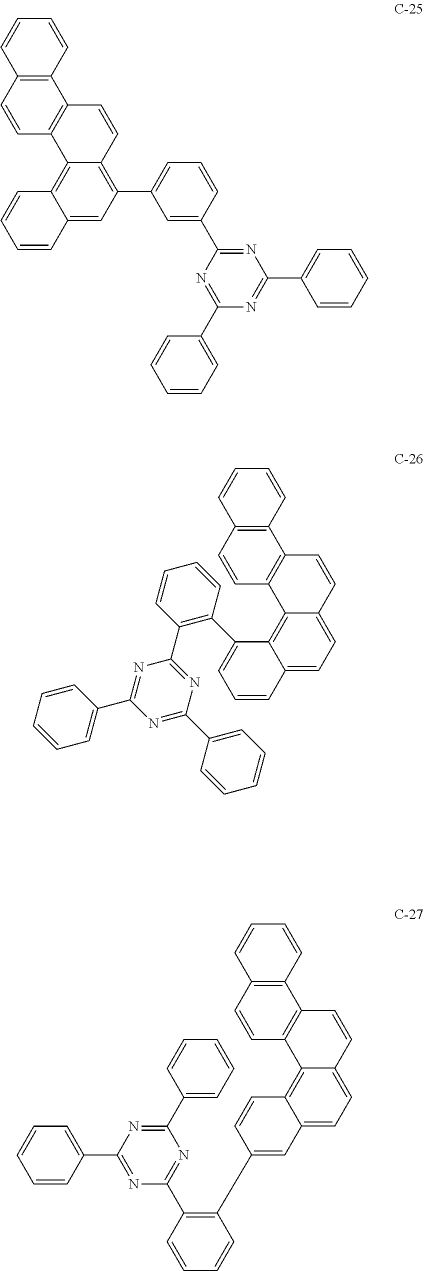 [SMILES: c1ccc(-c2nc(-c3ccccc3)nc(-c3cccc(-c4cc5ccccc5c5c4ccc4c6ccccc6ccc45)c3)n2)cc1 ; c1ccc(-c2nc(-c3ccccc3)nc(-c3ccccc3-c3cccc4ccc5ccc6c7ccccc7ccc6c5c34)n2)cc1 ; c1ccc(-c2nc(-c3ccccc3)nc(-c3ccccc3-c3ccc4c(ccc5ccc6c7ccccc7ccc6c54)c3)n2)cc1]

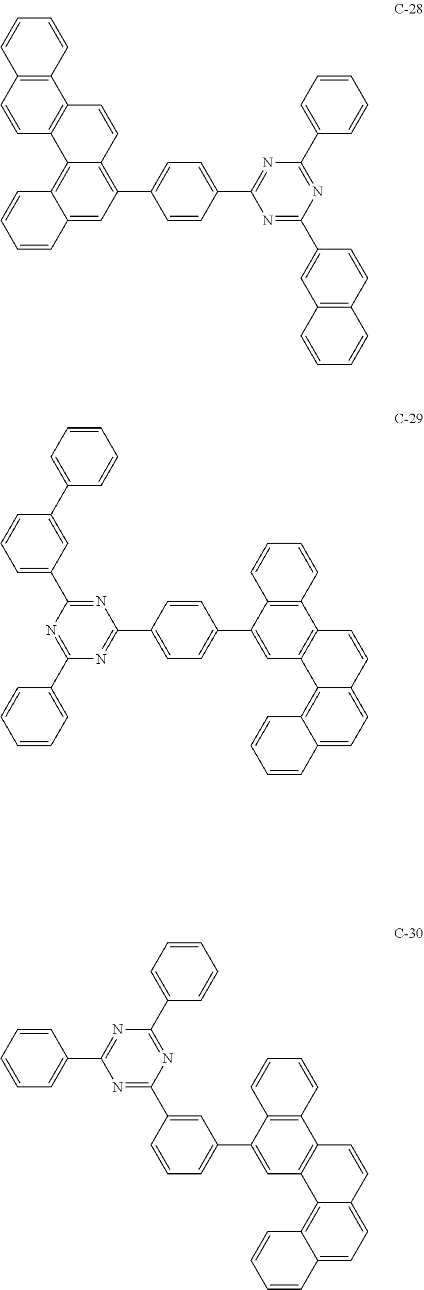 [SMILES: c1ccc(-c2nc(-c3ccc(-c4cc5ccccc5c5c4ccc4c6ccccc6ccc45)cc3)nc(-c3ccc4ccccc4c3)n2)cc1 ; c1ccc(-c2cccc(-c3nc(-c4ccccc4)nc(-c4ccc(-c5cc6c(ccc7ccc8ccccc8c76)c6ccccc56)cc4)n3)c2)cc1 ; c1ccc(-c2nc(-c3ccccc3)nc(-c3cccc(-c4cc5c(ccc6ccc7ccccc7c65)c5ccccc45)c3)n2)cc1]

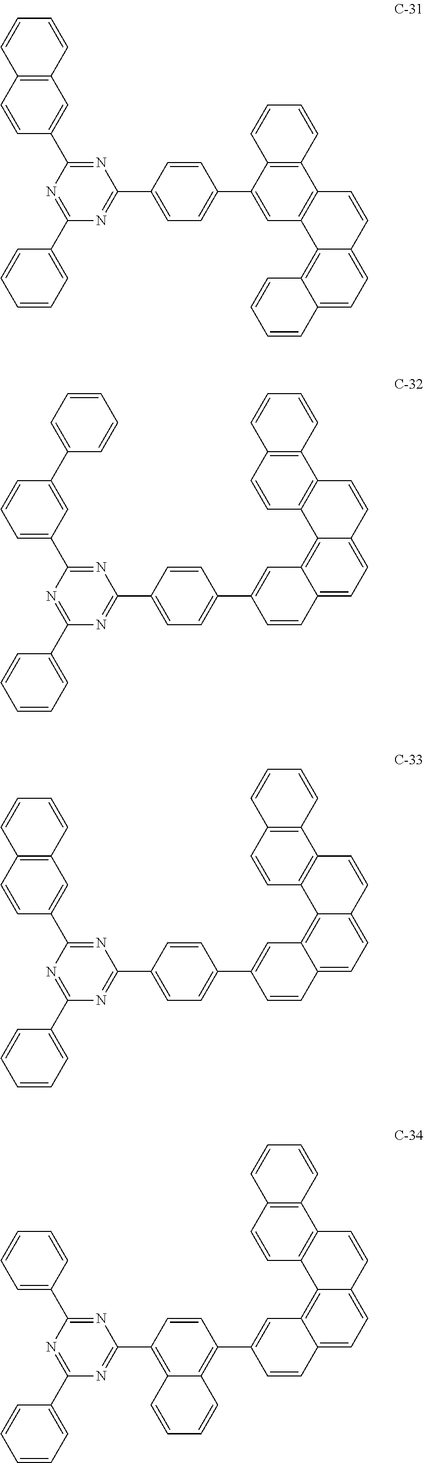 [SMILES: c1ccc(-c2nc(-c3ccc(-c4cc5c(ccc6ccc7ccccc7c65)c5ccccc45)cc3)nc(-c3ccc4ccccc4c3)n2)cc1 ; c1ccc(-c2cccc(-c3nc(-c4ccccc4)nc(-c4ccc(-c5ccc6ccc7ccc8c9ccccc9ccc8c7c6c5)cc4)n3)c2)cc1 ; c1ccc(-c2nc(-c3ccc(-c4ccc5ccc6ccc7c8ccccc8ccc7c6c5c4)cc3)nc(-c3ccc4ccccc4c3)n2)cc1 ; c1ccc(-c2nc(-c3ccccc3)nc(-c3ccc(-c4ccc5ccc6ccc7c8ccccc8ccc7c6c5c4)c4ccccc34)n2)cc1]

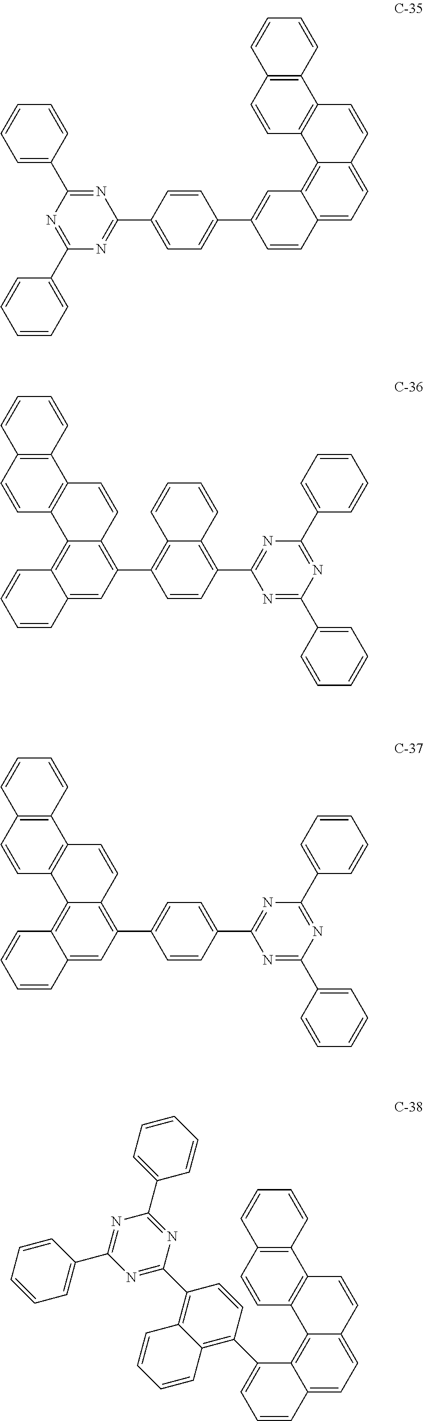 [SMILES: c1ccc(-c2nc(-c3ccccc3)nc(-c3ccc(-c4ccc5ccc6ccc7c8ccccc8ccc7c6c5c4)cc3)n2)cc1 ; c1ccc(-c2nc(-c3ccccc3)nc(-c3ccc(-c4cc5ccccc5c5c4ccc4c6ccccc6ccc45)c4ccccc34)n2)cc1 ; c1ccc(-c2nc(-c3ccccc3)nc(-c3ccc(-c4cc5ccccc5c5c4ccc4c6ccccc6ccc45)cc3)n2)cc1 ; c1ccc(-c2nc(-c3ccccc3)nc(-c3ccc(-c4cccc5ccc6ccc7c8ccccc8ccc7c6c45)c4ccccc34)n2)cc1]

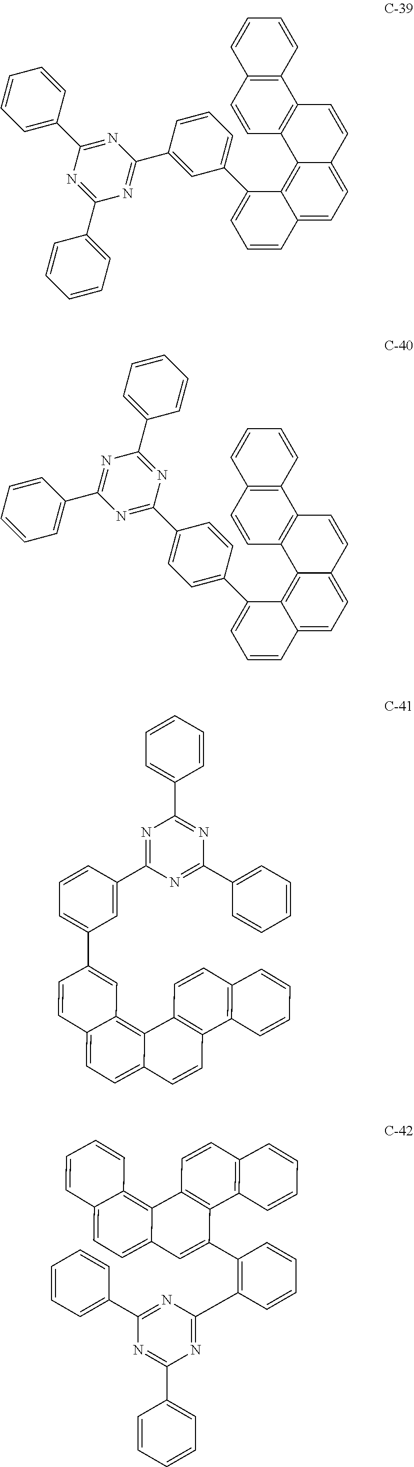 [SMILES: c1ccc(-c2nc(-c3ccccc3)nc(-c3cccc(-c4cccc5ccc6ccc7c8ccccc8ccc7c6c45)c3)n2)cc1 ; c1ccc(-c2nc(-c3ccccc3)nc(-c3ccc(-c4cccc5ccc6ccc7c8ccccc8ccc7c6c45)cc3)n2)cc1 ; c1ccc(-c2nc(-c3ccccc3)nc(-c3cccc(-c4ccc5ccc6ccc7c8ccccc8ccc7c6c5c4)c3)n2)cc1 ; c1ccc(-c2nc(-c3ccccc3)nc(-c3ccccc3-c3cc4ccc5ccccc5c4c4ccc5ccccc5c34)n2)cc1]

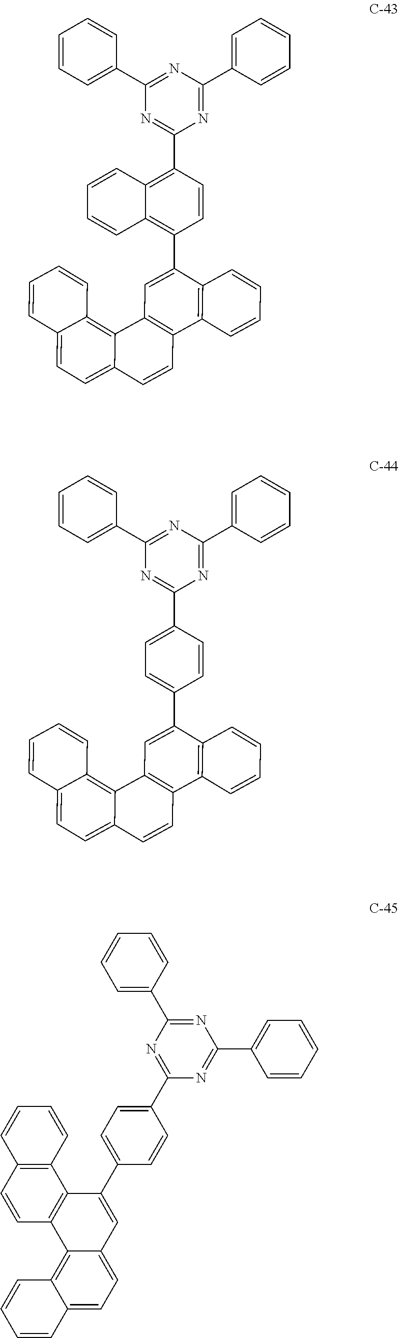 [SMILES: c1ccc(-c2nc(-c3ccccc3)nc(-c3ccc(-c4cc5c(ccc6ccc7ccccc7c65)c5ccccc45)c4ccccc34)n2)cc1 ; c1ccc(-c2nc(-c3ccccc3)nc(-c3ccc(-c4cc5c(ccc6ccc7ccccc7c65)c5ccccc45)cc3)n2)cc1 ; c1ccc(-c2nc(-c3ccccc3)nc(-c3ccc(-c4cc5ccc6ccccc6c5c5ccc6ccccc6c45)cc3)n2)cc1]

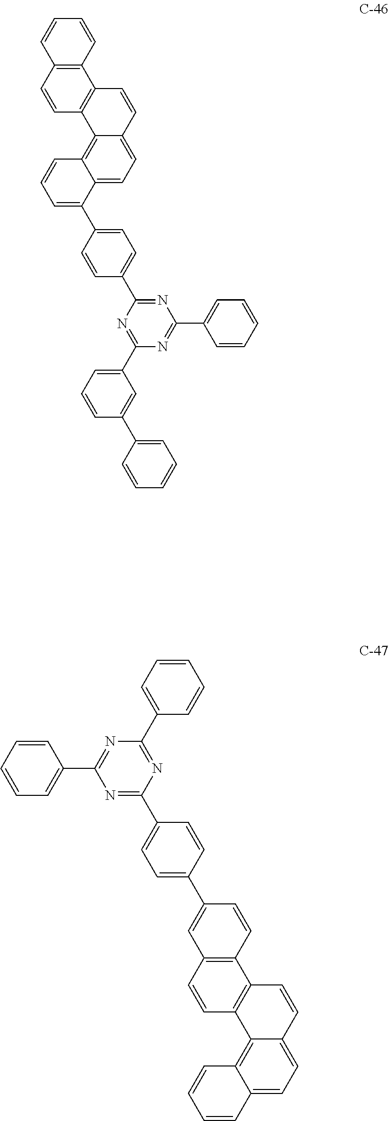 [SMILES: c1ccc(-c2cccc(-c3nc(-c4ccccc4)nc(-c4ccc(-c5cccc6c5ccc5ccc7c8ccccc8ccc7c56)cc4)n3)c2)cc1 ; c1ccc(-c2nc(-c3ccccc3)nc(-c3ccc(-c4ccc5c(ccc6c5ccc5ccc7ccccc7c56)c4)cc3)n2)cc1]

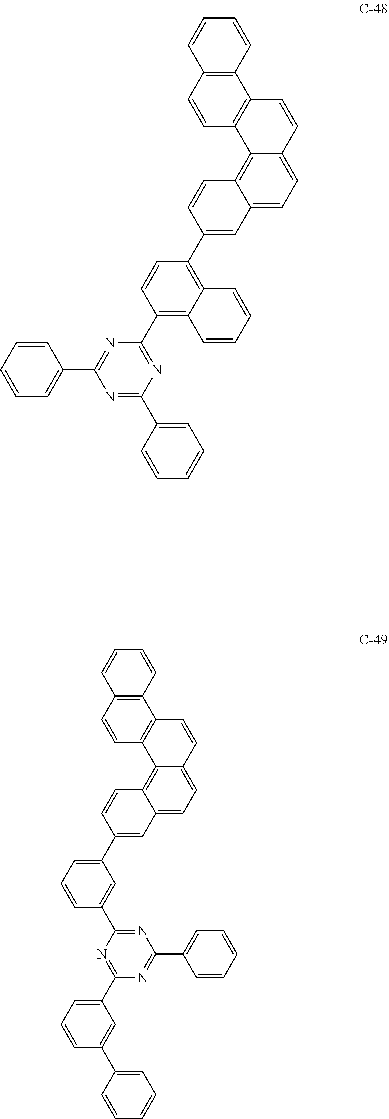 [SMILES: c1ccc(-c2nc(-c3ccccc3)nc(-c3ccc(-c4ccc5c(ccc6ccc7c8ccccc8ccc7c65)c4)c4ccccc34)n2)cc1 ; c1ccc(-c2cccc(-c3nc(-c4ccccc4)nc(-c4cccc(-c5ccc6c(ccc7ccc8c9ccccc9ccc8c76)c5)c4)n3)c2)cc1]

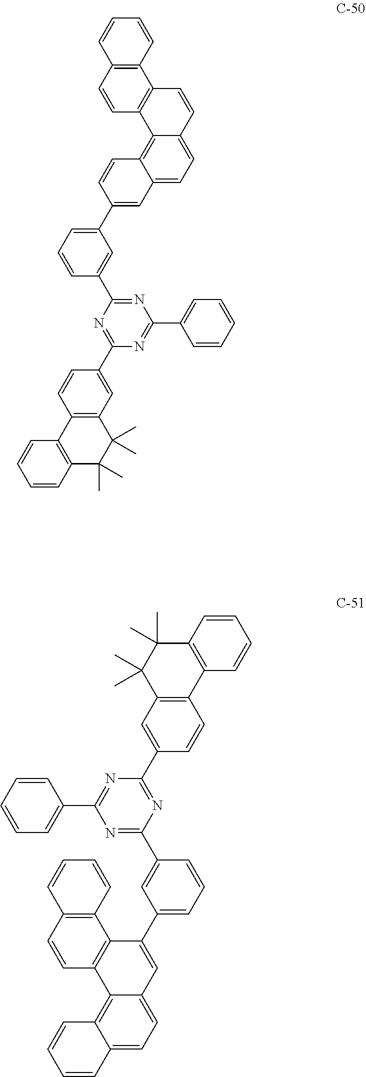 [SMILES: CC1(C)c2ccccc2-c2ccc(-c3nc(-c4ccccc4)nc(-c4cccc(-c5ccc6c(ccc7ccc8c9ccccc9ccc8c76)c5)c4)n3)cc2C1(C)C ; CC1(C)c2ccccc2-c2ccc(-c3nc(-c4ccccc4)nc(-c4cccc(-c5cc6ccc7ccccc7c6c6ccc7ccccc7c56)c4)n3)cc2C1(C)C]

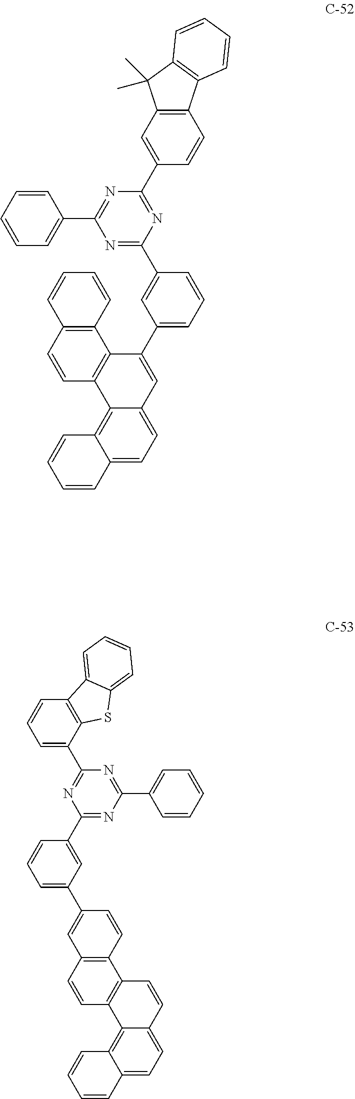 [SMILES: CC1(C)c2ccccc2-c2ccc(-c3nc(-c4ccccc4)nc(-c4cccc(-c5cc6ccc7ccccc7c6c6ccc7ccccc7c56)c4)n3)cc21 ; c1ccc(-c2nc(-c3cccc(-c4ccc5c(ccc6c5ccc5ccc7ccccc7c56)c4)c3)nc(-c3cccc4c3sc3ccccc34)n2)cc1]

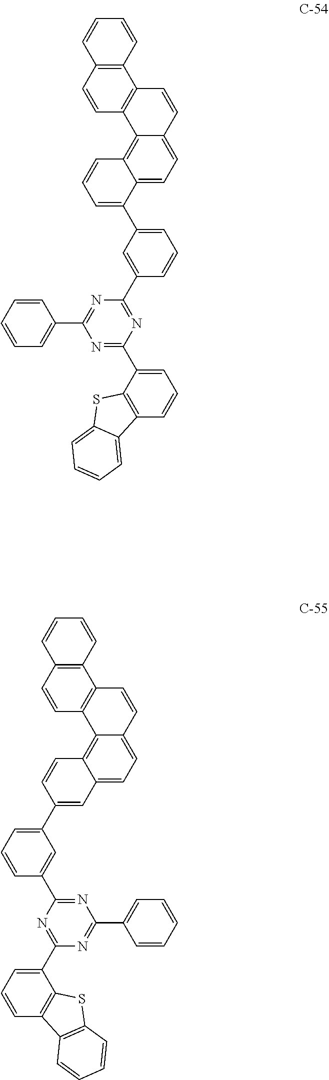 [SMILES: c1ccc(-c2nc(-c3cccc(-c4cccc5c4ccc4ccc6c7ccccc7ccc6c45)c3)nc(-c3cccc4c3sc3ccccc34)n2)cc1 ; c1ccc(-c2nc(-c3cccc(-c4ccc5c(ccc6ccc7c8ccccc8ccc7c65)c4)c3)nc(-c3cccc4c3sc3ccccc34)n2)cc1]

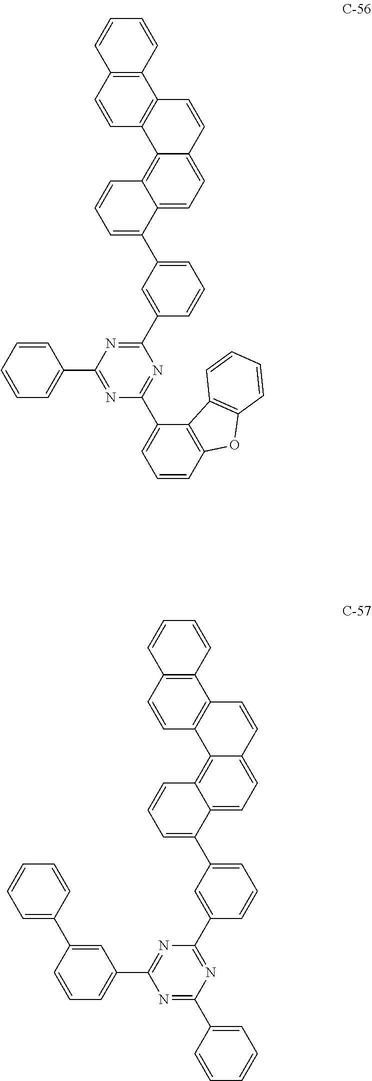 [SMILES: c1ccc(-c2nc(-c3cccc(-c4cccc5c4ccc4ccc6c7ccccc7ccc6c45)c3)nc(-c3cccc4oc5ccccc5c34)n2)cc1 ; c1ccc(-c2cccc(-c3nc(-c4ccccc4)nc(-c4cccc(-c5cccc6c5ccc5ccc7c8ccccc8ccc7c56)c4)n3)c2)cc1]

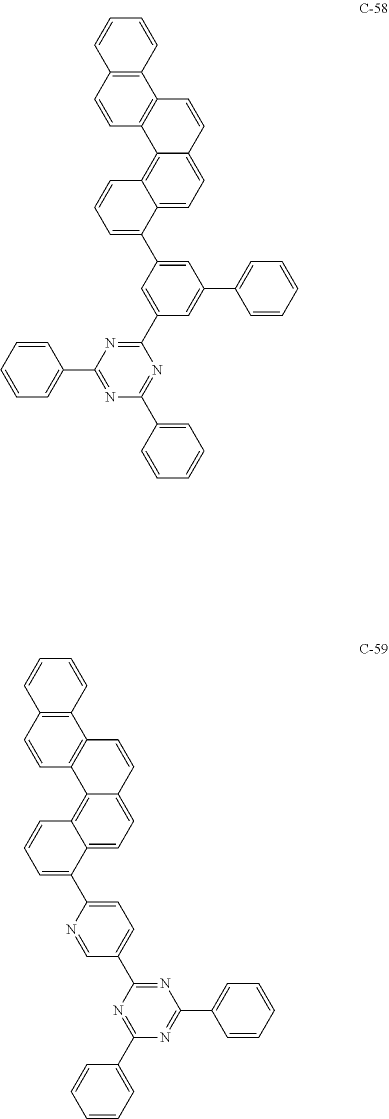 [SMILES: c1ccc(-c2cc(-c3nc(-c4ccccc4)nc(-c4ccccc4)n3)cc(-c3cccc4c3ccc3ccc5c6ccccc6ccc5c34)c2)cc1 ; c1ccc(-c2nc(-c3ccccc3)nc(-c3ccc(-c4cccc5c4ccc4ccc6c7ccccc7ccc6c45)nc3)n2)cc1]

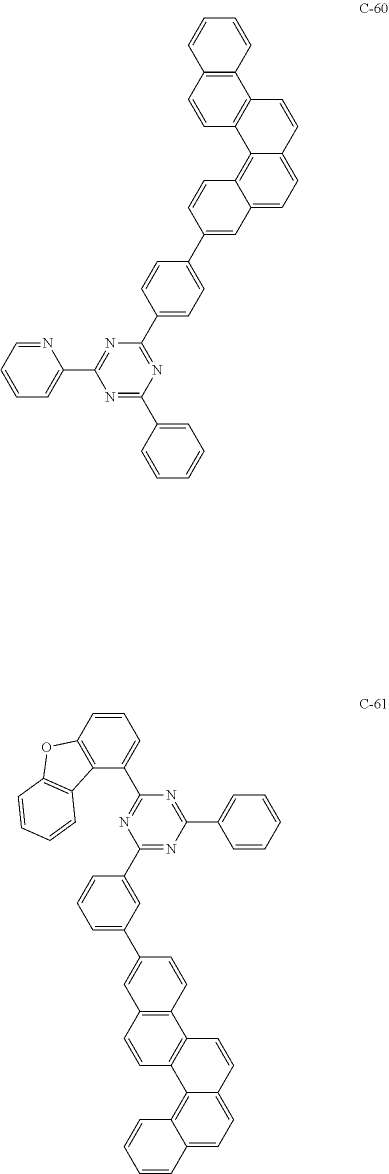 [SMILES: c1ccc(-c2nc(-c3ccc(-c4ccc5c(ccc6ccc7c8ccccc8ccc7c65)c4)cc3)nc(-c3ccccn3)n2)cc1 ; c1ccc(-c2nc(-c3cccc(-c4ccc5c(ccc6c5ccc5ccc7ccccc7c56)c4)c3)nc(-c3cccc4oc5ccccc5c34)n2)cc1]

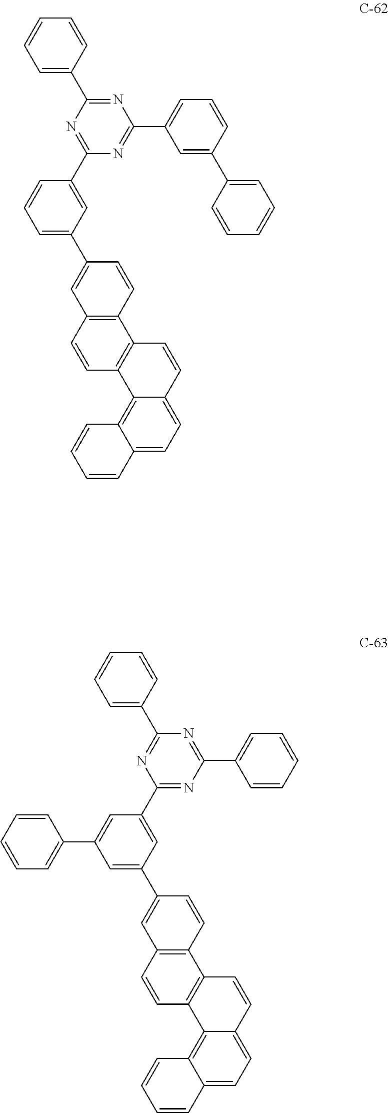 [SMILES: c1ccc(-c2cccc(-c3nc(-c4ccccc4)nc(-c4cccc(-c5ccc6c(ccc7c6ccc6ccc8ccccc8c67)c5)c4)n3)c2)cc1 ; c1ccc(-c2cc(-c3ccc4c(ccc5c4ccc4ccc6ccccc6c45)c3)cc(-c3nc(-c4ccccc4)nc(-c4ccccc4)n3)c2)cc1]

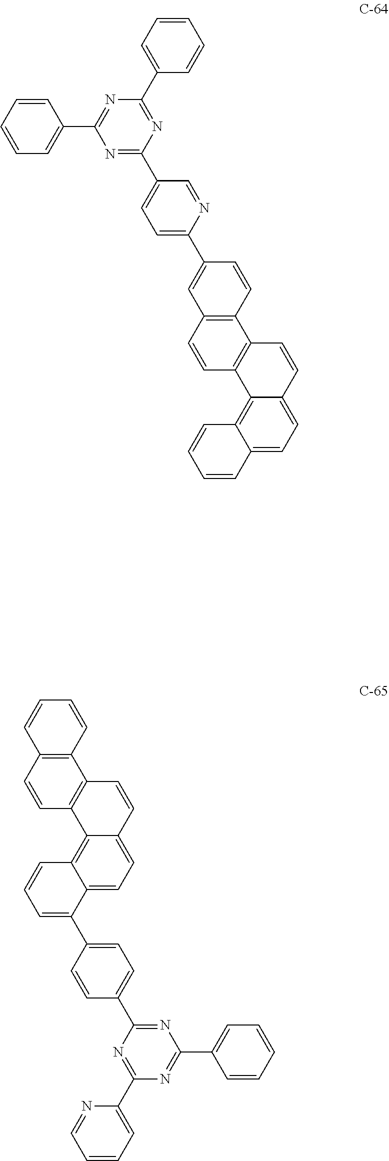 [SMILES: c1ccc(-c2nc(-c3ccccc3)nc(-c3ccc(-c4ccc5c(ccc6c5ccc5ccc7ccccc7c56)c4)nc3)n2)cc1 ; c1ccc(-c2nc(-c3ccc(-c4cccc5c4ccc4ccc6c7ccccc7ccc6c45)cc3)nc(-c3ccccn3)n2)cc1]

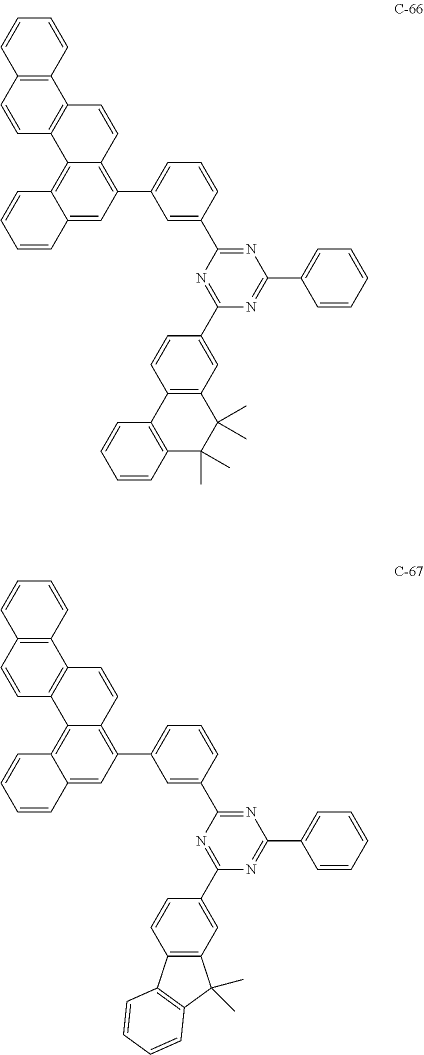 [SMILES: CC1(C)c2ccccc2-c2ccc(-c3nc(-c4ccccc4)nc(-c4cccc(-c5cc6ccccc6c6c5ccc5c7ccccc7ccc56)c4)n3)cc2C1(C)C ; CC1(C)c2ccccc2-c2ccc(-c3nc(-c4ccccc4)nc(-c4cccc(-c5cc6ccccc6c6c5ccc5c7ccccc7ccc56)c4)n3)cc21]

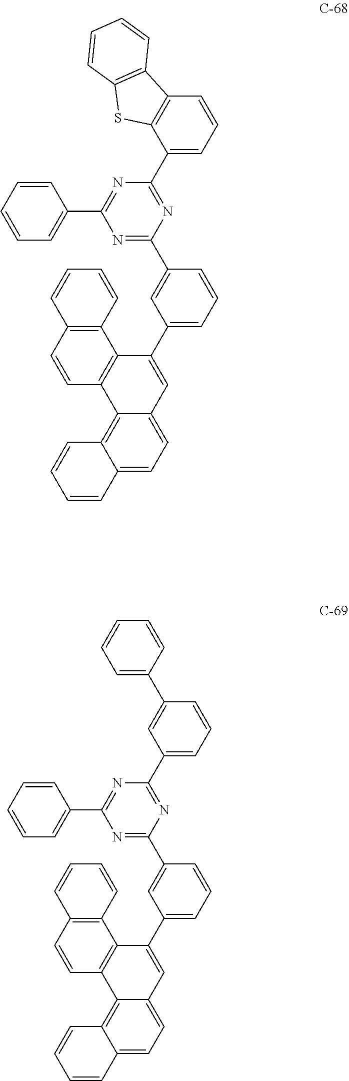 [SMILES: c1ccc(-c2nc(-c3cccc(-c4cc5ccc6ccccc6c5c5ccc6ccccc6c45)c3)nc(-c3cccc4c3sc3ccccc34)n2)cc1 ; c1ccc(-c2cccc(-c3nc(-c4ccccc4)nc(-c4cccc(-c5cc6ccc7ccccc7c6c6ccc7ccccc7c56)c4)n3)c2)cc1]

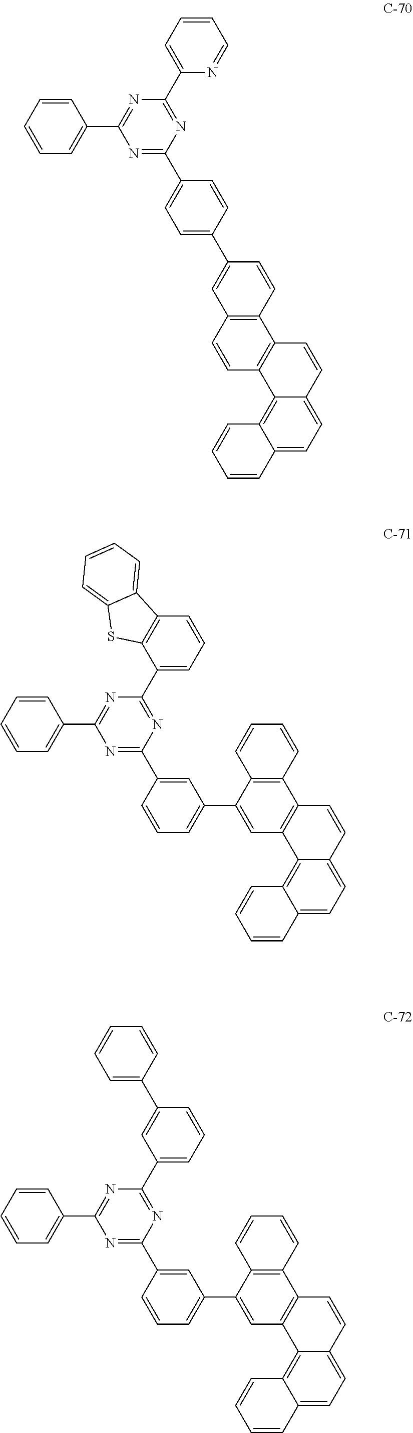 [SMILES: c1ccc(-c2nc(-c3ccc(-c4ccc5c(ccc6c5ccc5ccc7ccccc7c56)c4)cc3)nc(-c3ccccn3)n2)cc1 ; c1ccc(-c2nc(-c3cccc(-c4cc5c(ccc6ccc7ccccc7c65)c5ccccc45)c3)nc(-c3cccc4c3sc3ccccc34)n2)cc1 ; c1ccc(-c2cccc(-c3nc(-c4ccccc4)nc(-c4cccc(-c5cc6c(ccc7ccc8ccccc8c76)c6ccccc56)c4)n3)c2)cc1]

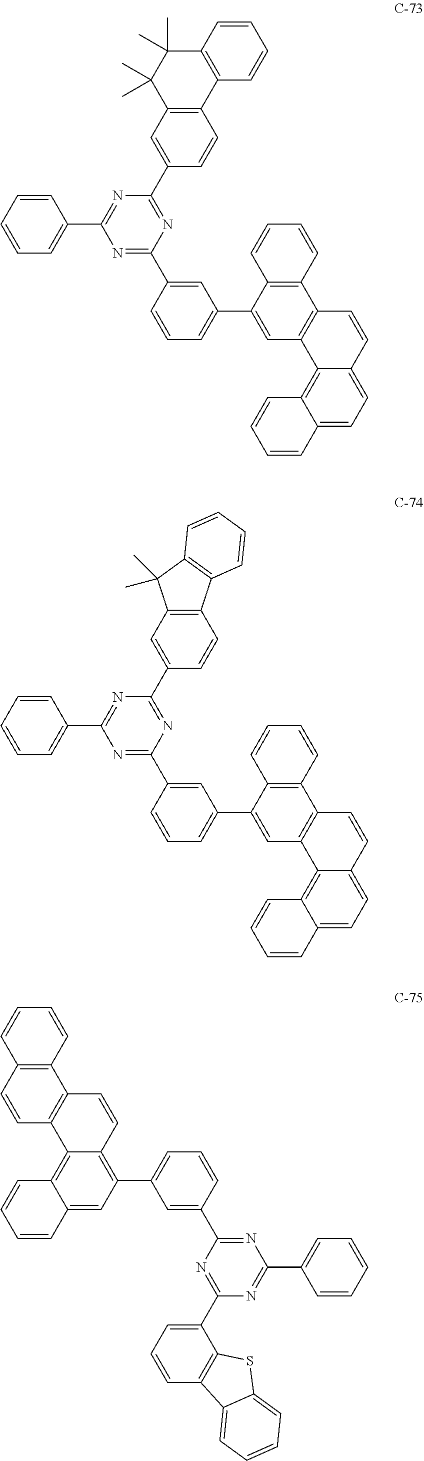 [SMILES: CC1(C)c2ccccc2-c2ccc(-c3nc(-c4ccccc4)nc(-c4cccc(-c5cc6c(ccc7ccc8ccccc8c76)c6ccccc56)c4)n3)cc2C1(C)C ; CC1(C)c2ccccc2-c2ccc(-c3nc(-c4ccccc4)nc(-c4cccc(-c5cc6c(ccc7ccc8ccccc8c76)c6ccccc56)c4)n3)cc21 ; c1ccc(-c2nc(-c3cccc(-c4cc5ccccc5c5c4ccc4c6ccccc6ccc45)c3)nc(-c3cccc4c3sc3ccccc34)n2)cc1]

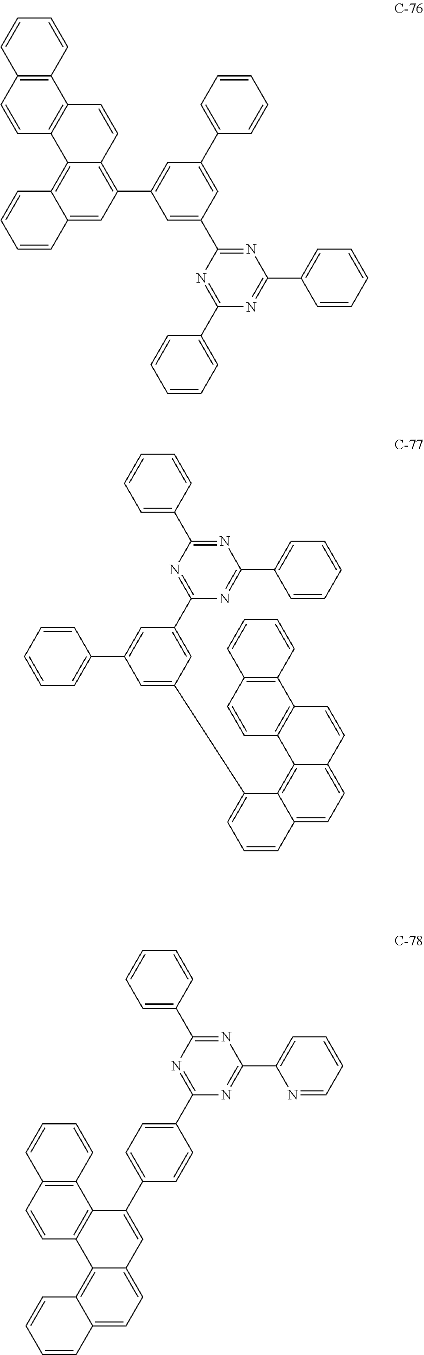 [SMILES: c1ccc(-c2cc(-c3nc(-c4ccccc4)nc(-c4ccccc4)n3)cc(-c3cc4ccccc4c4c3ccc3c5ccccc5ccc34)c2)cc1 ; c1ccc(-c2cc(-c3nc(-c4ccccc4)nc(-c4ccccc4)n3)cc(-c3cccc4ccc5ccc6c7ccccc7ccc6c5c34)c2)cc1 ; c1ccc(-c2nc(-c3ccc(-c4cc5ccc6ccccc6c5c5ccc6ccccc6c45)cc3)nc(-c3ccccn3)n2)cc1]

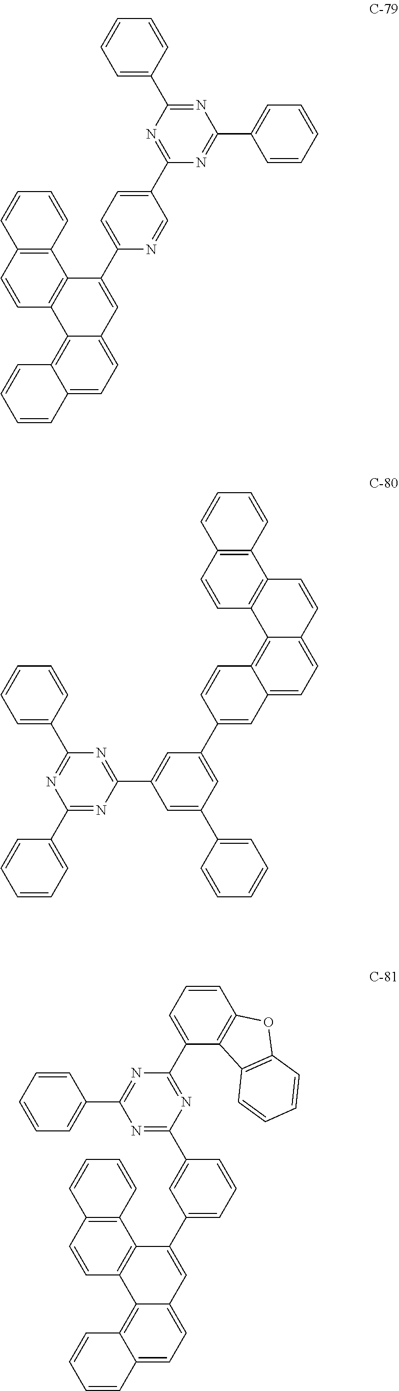 [SMILES: c1ccc(-c2nc(-c3ccccc3)nc(-c3ccc(-c4cc5ccc6ccccc6c5c5ccc6ccccc6c45)nc3)n2)cc1 ; c1ccc(-c2cc(-c3ccc4c(ccc5ccc6c7ccccc7ccc6c54)c3)cc(-c3nc(-c4ccccc4)nc(-c4ccccc4)n3)c2)cc1 ; c1ccc(-c2nc(-c3cccc(-c4cc5ccc6ccccc6c5c5ccc6ccccc6c45)c3)nc(-c3cccc4oc5ccccc5c34)n2)cc1]

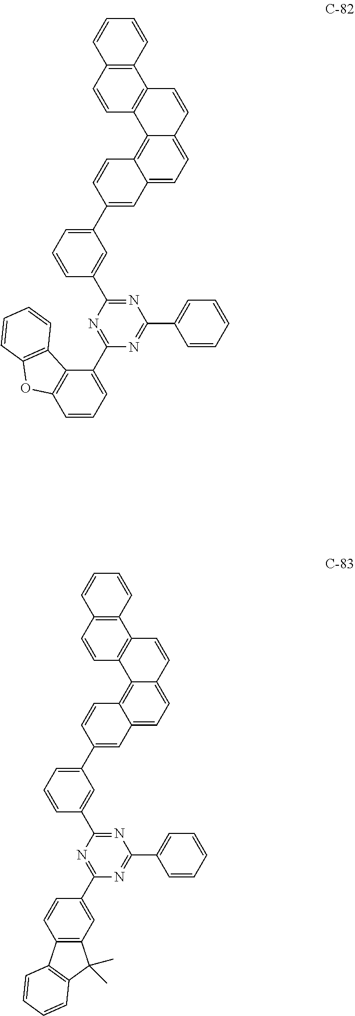 [SMILES: c1ccc(-c2nc(-c3cccc(-c4ccc5c(ccc6ccc7c8ccccc8ccc7c65)c4)c3)nc(-c3cccc4oc5ccccc5c34)n2)cc1 ; CC1(C)c2ccccc2-c2ccc(-c3nc(-c4ccccc4)nc(-c4cccc(-c5ccc6c(ccc7ccc8c9ccccc9ccc8c76)c5)c4)n3)cc21]

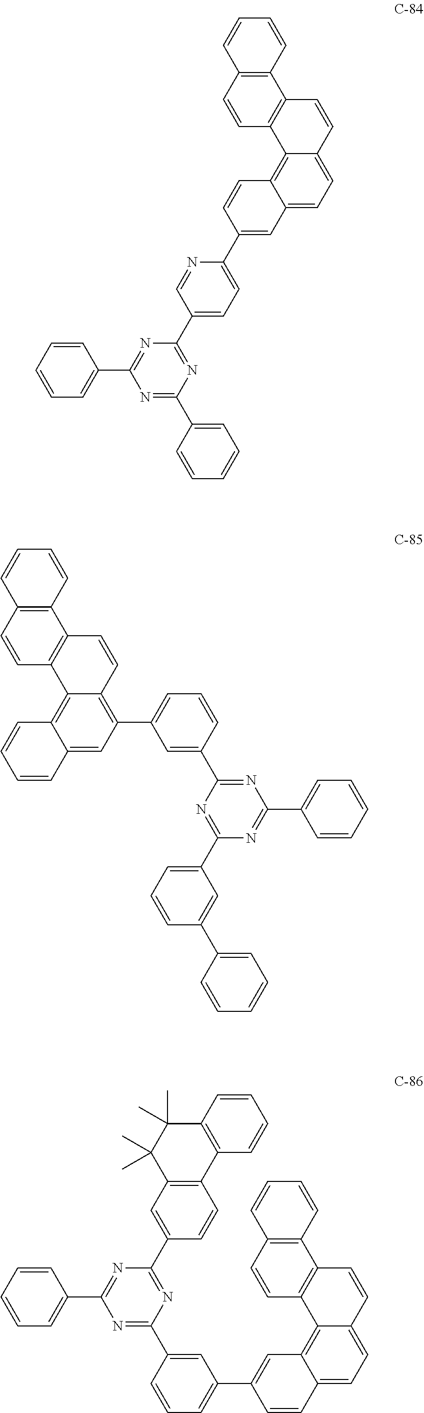 [SMILES: c1ccc(-c2nc(-c3ccccc3)nc(-c3ccc(-c4ccc5c(ccc6ccc7c8ccccc8ccc7c65)c4)nc3)n2)cc1 ; c1ccc(-c2cccc(-c3nc(-c4ccccc4)nc(-c4cccc(-c5cc6ccccc6c6c5ccc5c7ccccc7ccc56)c4)n3)c2)cc1 ; CC1(C)c2ccccc2-c2ccc(-c3nc(-c4ccccc4)nc(-c4cccc(-c5ccc6ccc7ccc8c9ccccc9ccc8c7c6c5)c4)n3)cc2C1(C)C]

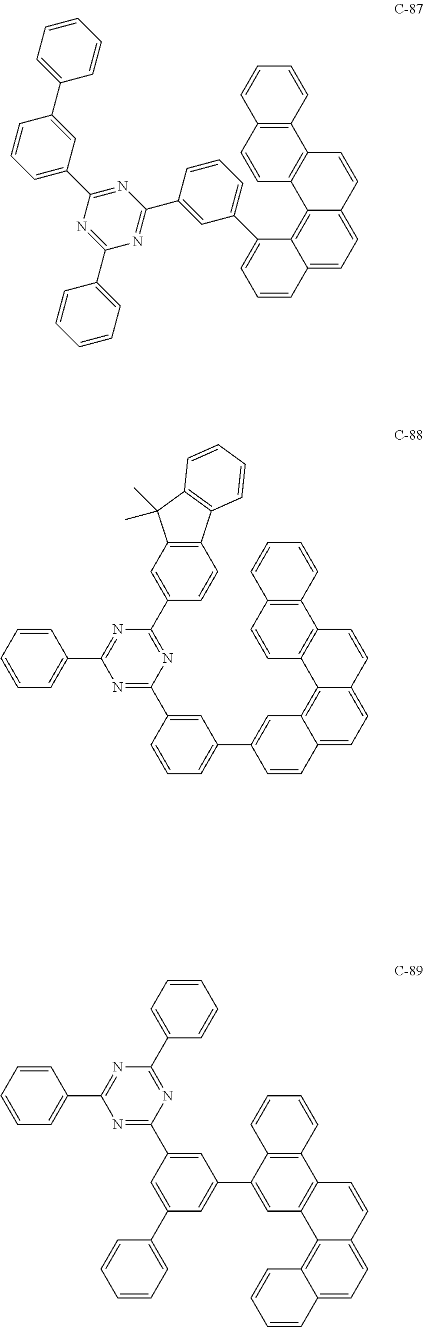 [SMILES: c1ccc(-c2cccc(-c3nc(-c4ccccc4)nc(-c4cccc(-c5cccc6ccc7ccc8c9ccccc9ccc8c7c56)c4)n3)c2)cc1 ; CC1(C)c2ccccc2-c2ccc(-c3nc(-c4ccccc4)nc(-c4cccc(-c5ccc6ccc7ccc8c9ccccc9ccc8c7c6c5)c4)n3)cc21 ; c1ccc(-c2cc(-c3nc(-c4ccccc4)nc(-c4ccccc4)n3)cc(-c3cc4c(ccc5ccc6ccccc6c54)c4ccccc34)c2)cc1]

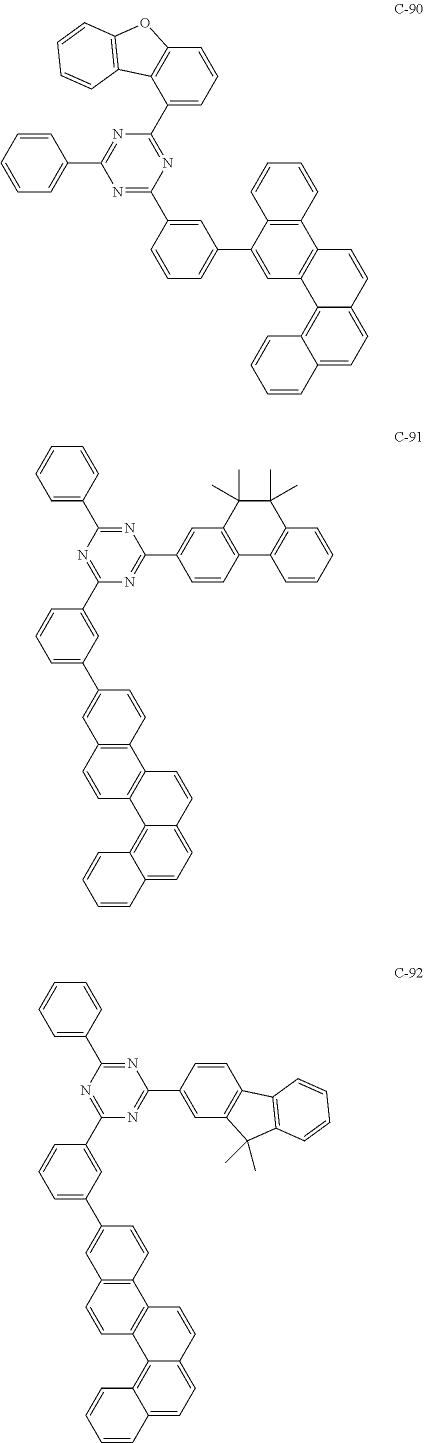 [SMILES: c1ccc(-c2nc(-c3cccc(-c4cc5c(ccc6ccc7ccccc7c65)c5ccccc45)c3)nc(-c3cccc4oc5ccccc5c34)n2)cc1 ; CC1(C)c2ccccc2-c2ccc(-c3nc(-c4ccccc4)nc(-c4cccc(-c5ccc6c(ccc7c6ccc6ccc8ccccc8c67)c5)c4)n3)cc2C1(C)C ; CC1(C)c2ccccc2-c2ccc(-c3nc(-c4ccccc4)nc(-c4cccc(-c5ccc6c(ccc7c6ccc6ccc8ccccc8c67)c5)c4)n3)cc21]

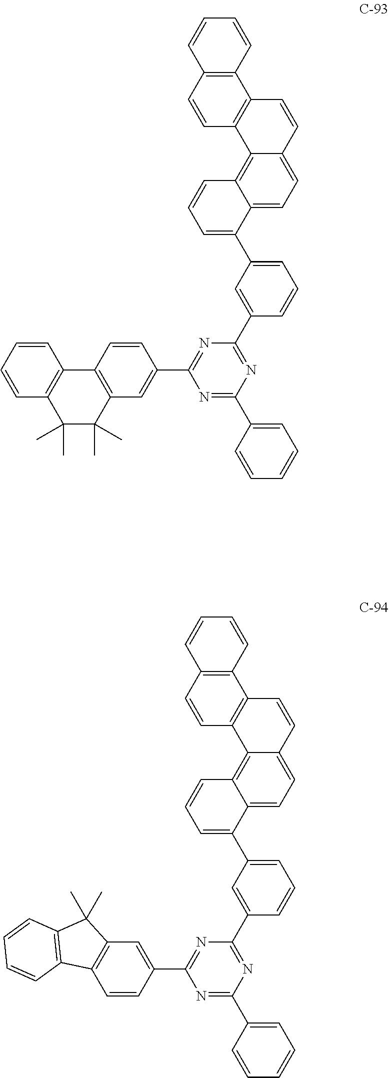 [SMILES: CC1(C)c2ccccc2-c2ccc(-c3nc(-c4ccccc4)nc(-c4cccc(-c5cccc6c5ccc5ccc7c8ccccc8ccc7c56)c4)n3)cc2C1(C)C ; CC1(C)c2ccccc2-c2ccc(-c3nc(-c4ccccc4)nc(-c4cccc(-c5cccc6c5ccc5ccc7c8ccccc8ccc7c56)c4)n3)cc21]

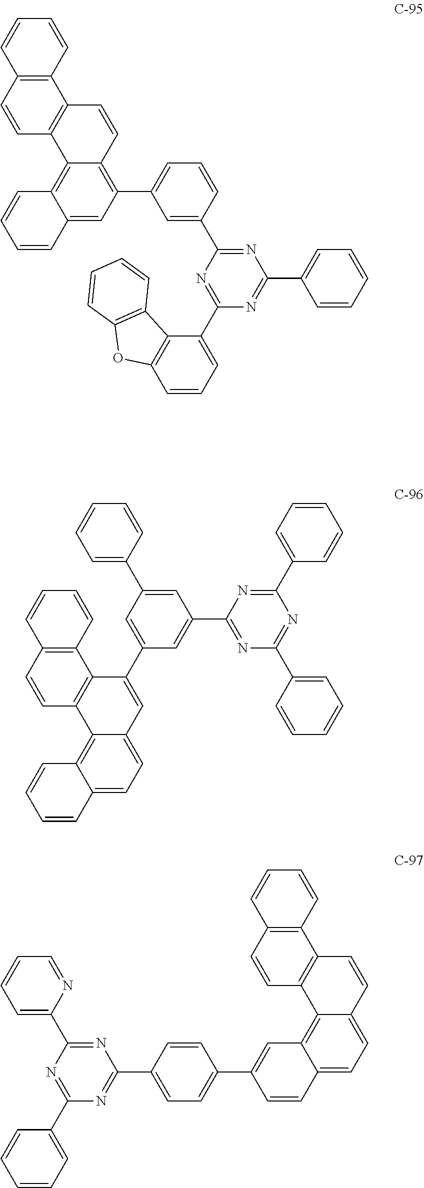 [SMILES: c1ccc(-c2nc(-c3cccc(-c4cc5ccccc5c5c4ccc4c6ccccc6ccc45)c3)nc(-c3cccc4oc5ccccc5c34)n2)cc1 ; c1ccc(-c2cc(-c3nc(-c4ccccc4)nc(-c4ccccc4)n3)cc(-c3cc4ccc5ccccc5c4c4ccc5ccccc5c34)c2)cc1 ; c1ccc(-c2nc(-c3ccc(-c4ccc5ccc6ccc7c8ccccc8ccc7c6c5c4)cc3)nc(-c3ccccn3)n2)cc1]

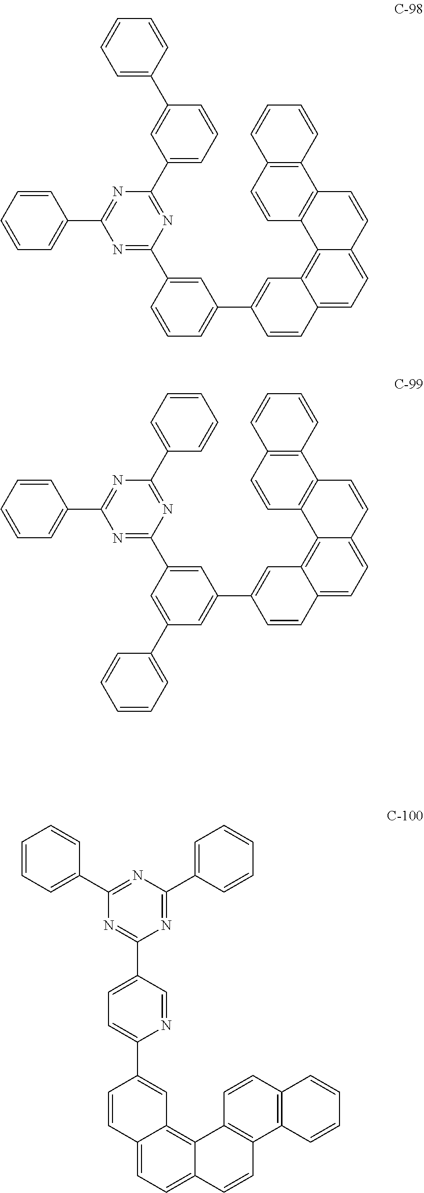 [SMILES: c1ccc(-c2cccc(-c3nc(-c4ccccc4)nc(-c4cccc(-c5ccc6ccc7ccc8c9ccccc9ccc8c7c6c5)c4)n3)c2)cc1 ; c1ccc(-c2cc(-c3ccc4ccc5ccc6c7ccccc7ccc6c5c4c3)cc(-c3nc(-c4ccccc4)nc(-c4ccccc4)n3)c2)cc1 ; c1ccc(-c2nc(-c3ccccc3)nc(-c3ccc(-c4ccc5ccc6ccc7c8ccccc8ccc7c6c5c4)nc3)n2)cc1]

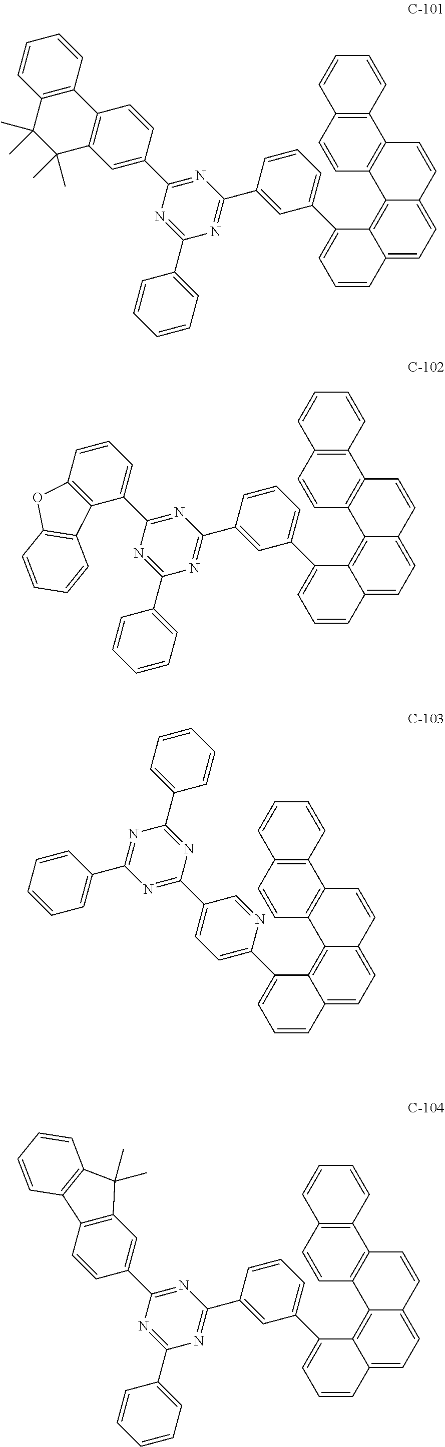 [SMILES: CC1(C)c2ccccc2-c2ccc(-c3nc(-c4ccccc4)nc(-c4cccc(-c5cccc6ccc7ccc8c9ccccc9ccc8c7c56)c4)n3)cc2C1(C)C ; c1ccc(-c2nc(-c3cccc(-c4cccc5ccc6ccc7c8ccccc8ccc7c6c45)c3)nc(-c3cccc4oc5ccccc5c34)n2)cc1 ; c1ccc(-c2nc(-c3ccccc3)nc(-c3ccc(-c4cccc5ccc6ccc7c8ccccc8ccc7c6c45)nc3)n2)cc1 ; CC1(C)c2ccccc2-c2ccc(-c3nc(-c4ccccc4)nc(-c4cccc(-c5cccc6ccc7ccc8c9ccccc9ccc8c7c56)c4)n3)cc21]

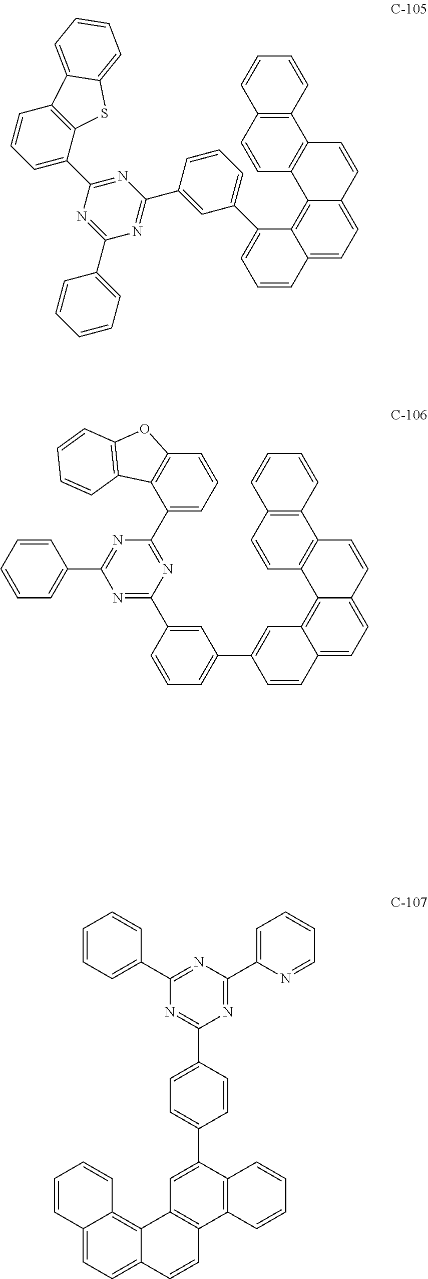 [SMILES: c1ccc(-c2nc(-c3cccc(-c4cccc5ccc6ccc7c8ccccc8ccc7c6c45)c3)nc(-c3cccc4c3sc3ccccc34)n2)cc1 ; c1ccc(-c2nc(-c3cccc(-c4ccc5ccc6ccc7c8ccccc8ccc7c6c5c4)c3)nc(-c3cccc4oc5ccccc5c34)n2)cc1 ; c1ccc(-c2nc(-c3ccc(-c4cc5c(ccc6ccc7ccccc7c65)c5ccccc45)cc3)nc(-c3ccccn3)n2)cc1]

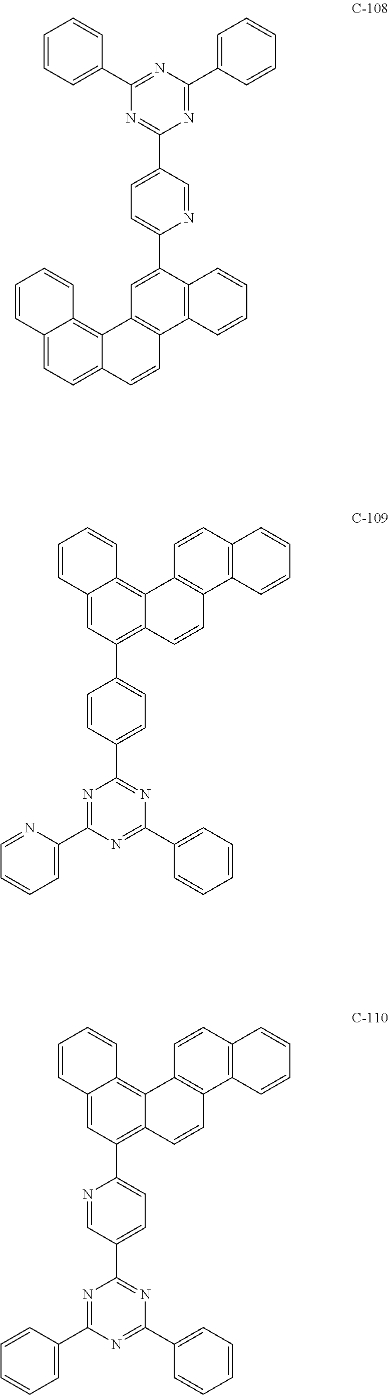 [SMILES: c1ccc(-c2nc(-c3ccccc3)nc(-c3ccc(-c4cc5c(ccc6ccc7ccccc7c65)c5ccccc45)nc3)n2)cc1 ; c1ccc(-c2nc(-c3ccc(-c4cc5ccccc5c5c4ccc4c6ccccc6ccc45)cc3)nc(-c3ccccn3)n2)cc1 ; c1ccc(-c2nc(-c3ccccc3)nc(-c3ccc(-c4cc5ccccc5c5c4ccc4c6ccccc6ccc45)nc3)n2)cc1]

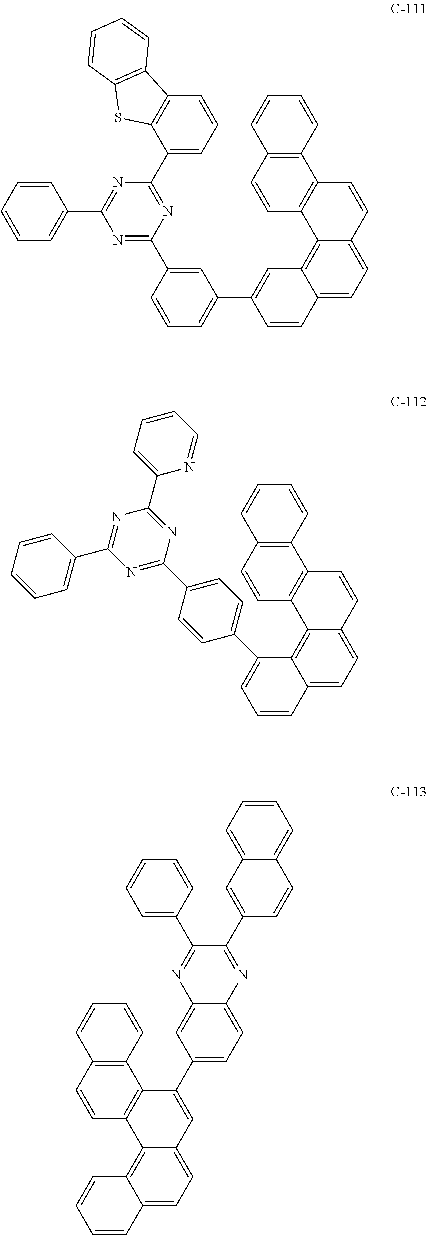 [SMILES: c1ccc(-c2nc(-c3cccc(-c4ccc5ccc6ccc7c8ccccc8ccc7c6c5c4)c3)nc(-c3cccc4c3sc3ccccc34)n2)cc1 ; c1ccc(-c2nc(-c3ccc(-c4cccc5ccc6ccc7c8ccccc8ccc7c6c45)cc3)nc(-c3ccccn3)n2)cc1 ; c1ccc(-c2nc3cc(-c4cc5ccc6ccccc6c5c5ccc6ccccc6c45)ccc3nc2-c2ccc3ccccc3c2)cc1]

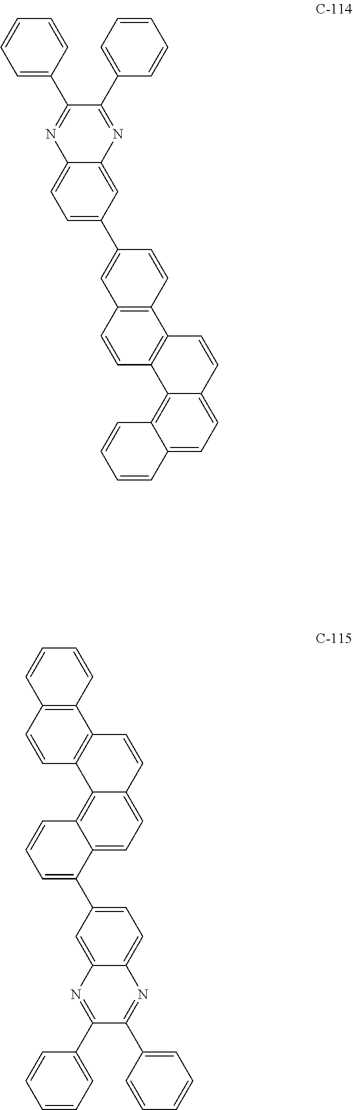 [SMILES: c1ccc(-c2nc3ccc(-c4ccc5c(ccc6c5ccc5ccc7ccccc7c56)c4)cc3nc2-c2ccccc2)cc1 ; c1ccc(-c2nc3ccc(-c4cccc5c4ccc4ccc6c7ccccc7ccc6c45)cc3nc2-c2ccccc2)cc1]

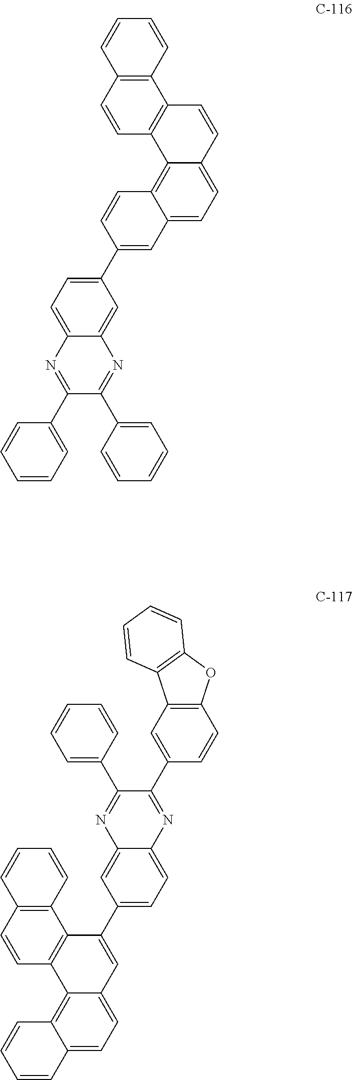 [SMILES: c1ccc(-c2nc3ccc(-c4ccc5c(ccc6ccc7c8ccccc8ccc7c65)c4)cc3nc2-c2ccccc2)cc1 ; c1ccc(-c2nc3cc(-c4cc5ccc6ccccc6c5c5ccc6ccccc6c45)ccc3nc2-c2ccc3oc4ccccc4c3c2)cc1]

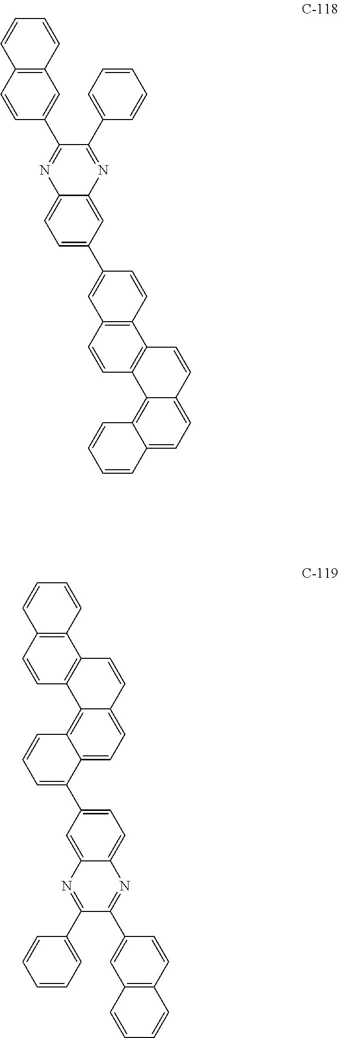 [SMILES: c1ccc(-c2nc3cc(-c4ccc5c(ccc6c5ccc5ccc7ccccc7c56)c4)ccc3nc2-c2ccc3ccccc3c2)cc1 ; c1ccc(-c2nc3cc(-c4cccc5c4ccc4ccc6c7ccccc7ccc6c45)ccc3nc2-c2ccc3ccccc3c2)cc1]

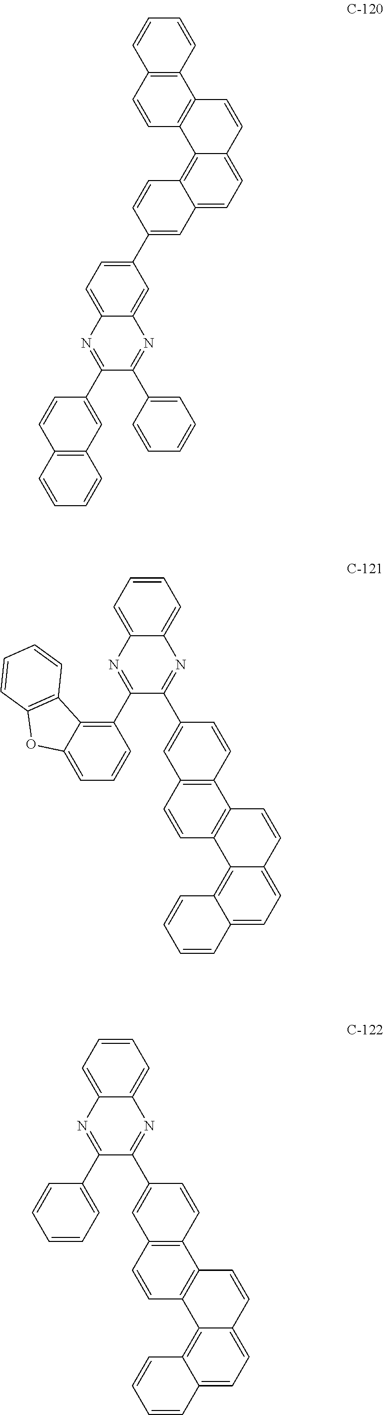 [SMILES: c1ccc(-c2nc3cc(-c4ccc5c(ccc6ccc7c8ccccc8ccc7c65)c4)ccc3nc2-c2ccc3ccccc3c2)cc1 ; c1ccc2c(c1)ccc1ccc3c4ccc(-c5nc6ccccc6nc5-c5cccc6oc7ccccc7c56)cc4ccc3c12 ; c1ccc(-c2nc3ccccc3nc2-c2ccc3c(ccc4c3ccc3ccc5ccccc5c34)c2)cc1]

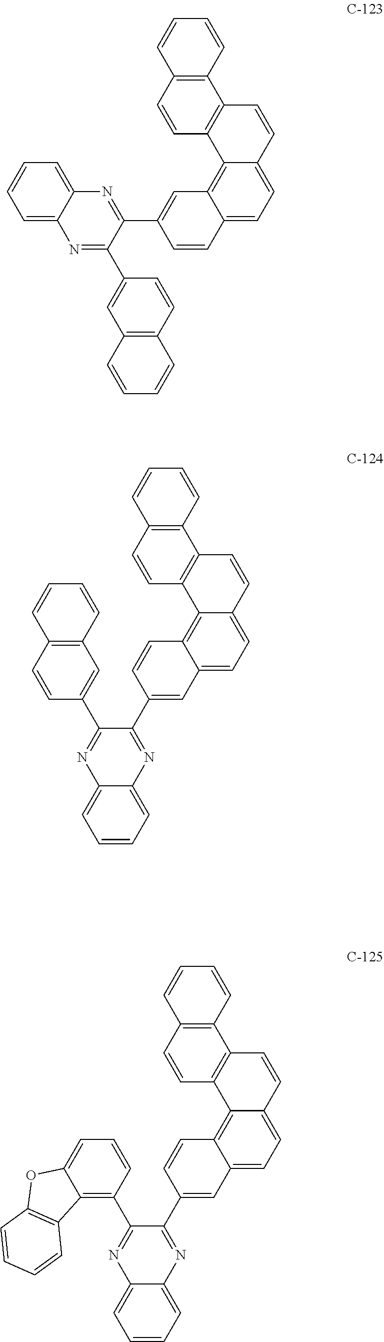 [SMILES: c1ccc2cc(-c3nc4ccccc4nc3-c3ccc4ccc5ccc6c7ccccc7ccc6c5c4c3)ccc2c1 ; c1ccc2cc(-c3nc4ccccc4nc3-c3ccc4c(ccc5ccc6c7ccccc7ccc6c54)c3)ccc2c1 ; c1ccc2c(c1)ccc1c2ccc2ccc3cc(-c4nc5ccccc5nc4-c4cccc5oc6ccccc6c45)ccc3c21]

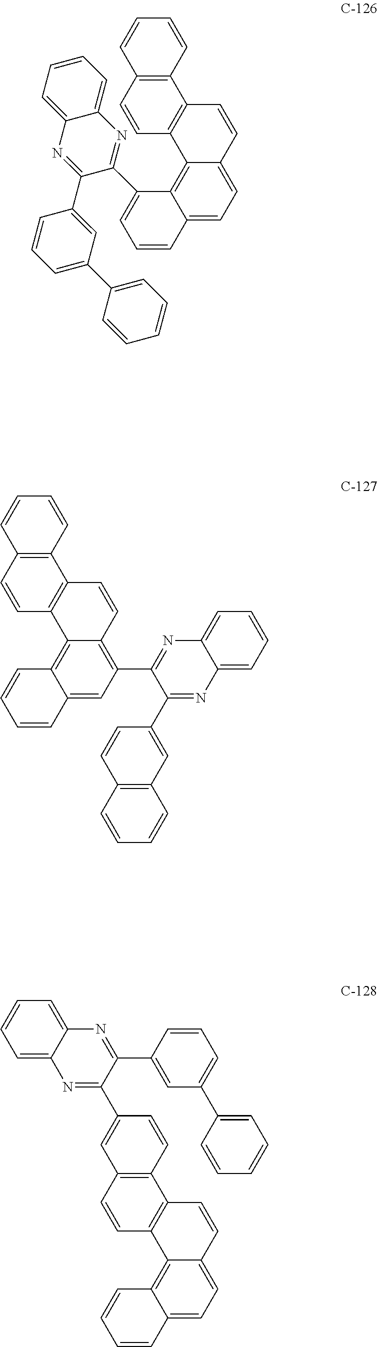 [SMILES: c1ccc(-c2cccc(-c3nc4ccccc4nc3-c3cccc4ccc5ccc6c7ccccc7ccc6c5c34)c2)cc1 ; c1ccc2cc(-c3nc4ccccc4nc3-c3cc4ccccc4c4c3ccc3c5ccccc5ccc34)ccc2c1 ; c1ccc(-c2cccc(-c3nc4ccccc4nc3-c3ccc4c(ccc5c4ccc4ccc6ccccc6c45)c3)c2)cc1]

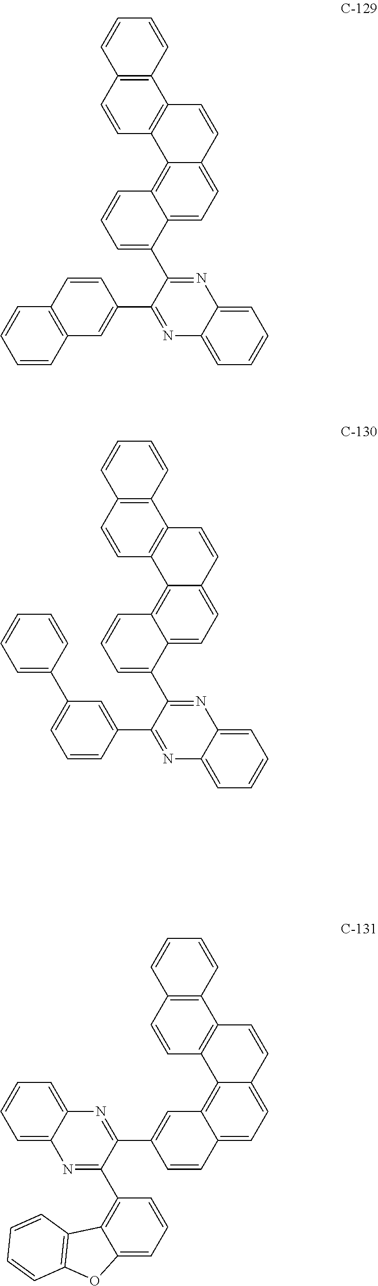 [SMILES: c1ccc2cc(-c3nc4ccccc4nc3-c3cccc4c3ccc3ccc5c6ccccc6ccc5c34)ccc2c1 ; c1ccc(-c2cccc(-c3nc4ccccc4nc3-c3cccc4c3ccc3ccc5c6ccccc6ccc5c34)c2)cc1 ; c1ccc2c(c1)ccc1c2ccc2ccc3ccc(-c4nc5ccccc5nc4-c4cccc5oc6ccccc6c45)cc3c21]

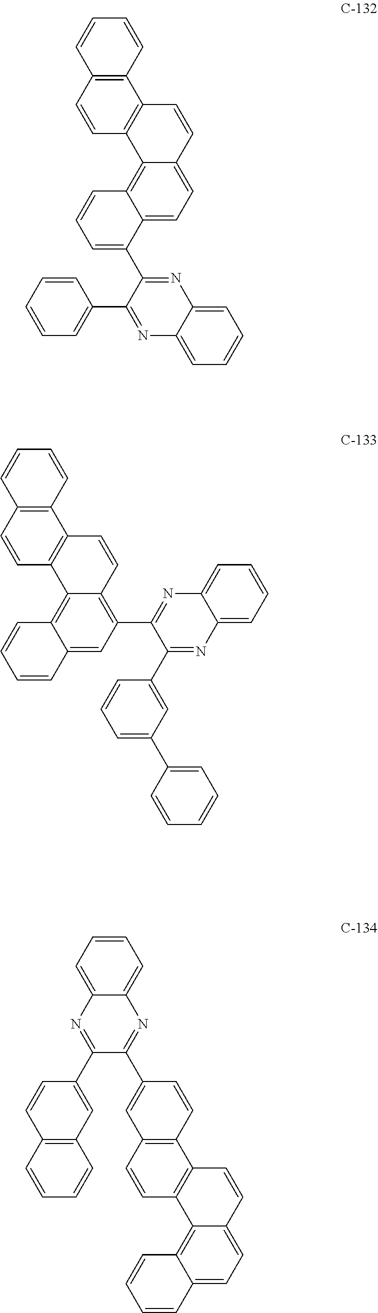 [SMILES: c1ccc(-c2nc3ccccc3nc2-c2cccc3c2ccc2ccc4c5ccccc5ccc4c23)cc1 ; c1ccc(-c2cccc(-c3nc4ccccc4nc3-c3cc4ccccc4c4c3ccc3c5ccccc5ccc34)c2)cc1 ; c1ccc2cc(-c3nc4ccccc4nc3-c3ccc4c(ccc5c4ccc4ccc6ccccc6c45)c3)ccc2c1]

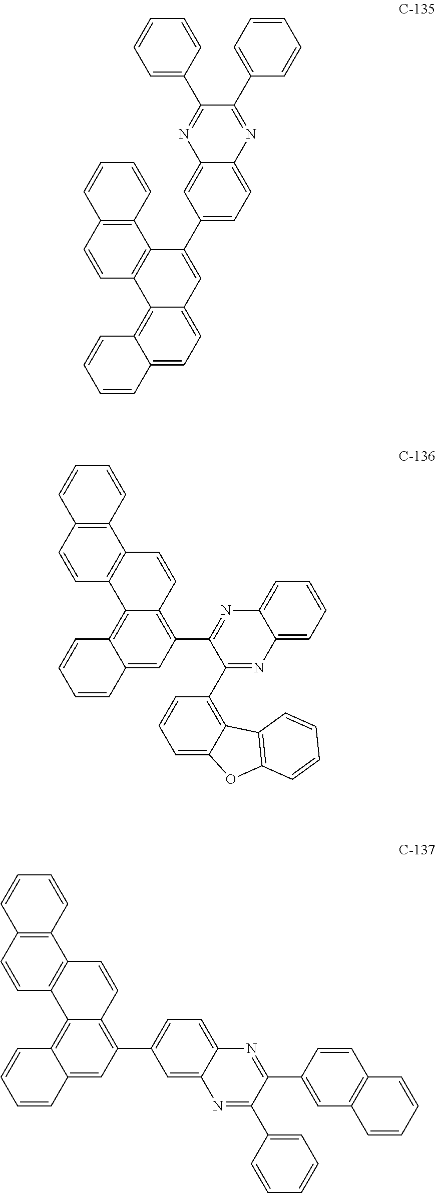 [SMILES: c1ccc(-c2nc3ccc(-c4cc5ccc6ccccc6c5c5ccc6ccccc6c45)cc3nc2-c2ccccc2)cc1 ; c1ccc2c(c1)ccc1c2ccc2c(-c3nc4ccccc4nc3-c3cccc4oc5ccccc5c34)cc3ccccc3c21 ; c1ccc(-c2nc3cc(-c4cc5ccccc5c5c4ccc4c6ccccc6ccc45)ccc3nc2-c2ccc3ccccc3c2)cc1]

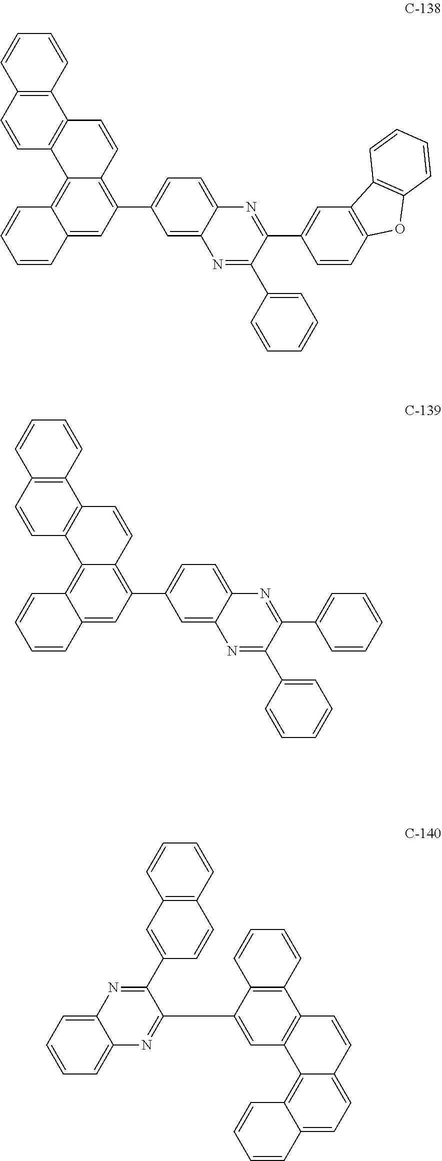 [SMILES: c1ccc(-c2nc3cc(-c4cc5ccccc5c5c4ccc4c6ccccc6ccc45)ccc3nc2-c2ccc3oc4ccccc4c3c2)cc1 ; c1ccc(-c2nc3ccc(-c4cc5ccccc5c5c4ccc4c6ccccc6ccc45)cc3nc2-c2ccccc2)cc1 ; c1ccc2cc(-c3nc4ccccc4nc3-c3cc4c(ccc5ccc6ccccc6c54)c4ccccc34)ccc2c1]

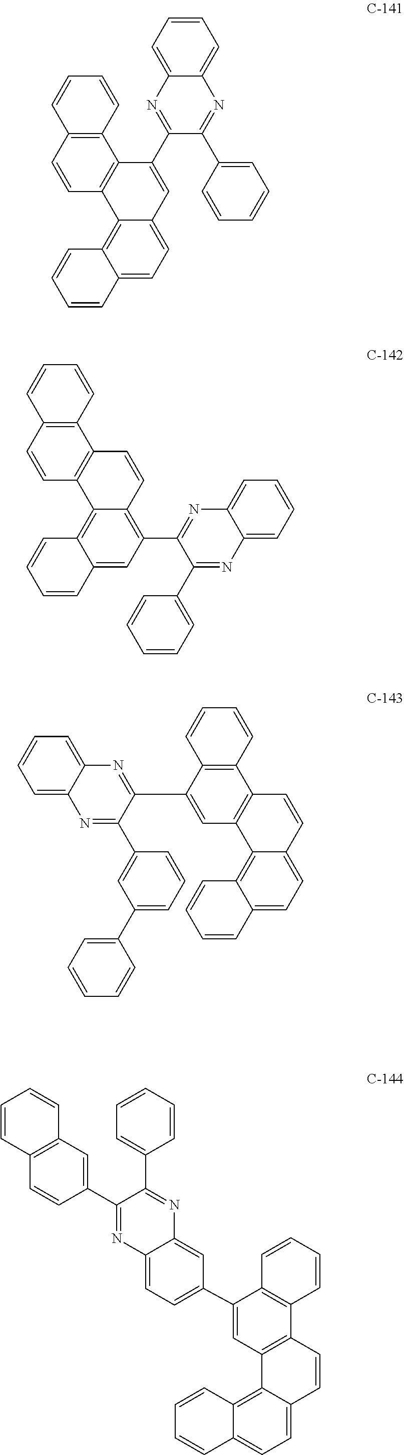 [SMILES: c1ccc(-c2nc3ccccc3nc2-c2cc3ccc4ccccc4c3c3ccc4ccccc4c23)cc1 ; c1ccc(-c2nc3ccccc3nc2-c2cc3ccccc3c3c2ccc2c4ccccc4ccc23)cc1 ; c1ccc(-c2cccc(-c3nc4ccccc4nc3-c3cc4c(ccc5ccc6ccccc6c54)c4ccccc34)c2)cc1 ; c1ccc(-c2nc3cc(-c4cc5c(ccc6ccc7ccccc7c65)c5ccccc45)ccc3nc2-c2ccc3ccccc3c2)cc1]

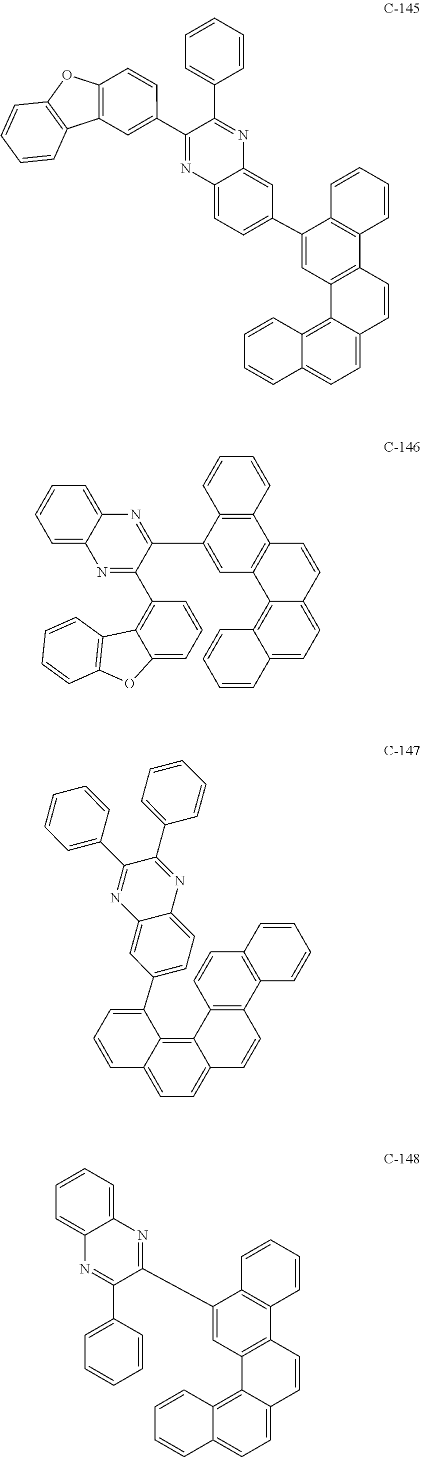 [SMILES: c1ccc(-c2nc3cc(-c4cc5c(ccc6ccc7ccccc7c65)c5ccccc45)ccc3nc2-c2ccc3oc4ccccc4c3c2)cc1 ; c1ccc2c(c1)ccc1ccc3c4ccccc4c(-c4nc5ccccc5nc4-c4cccc5oc6ccccc6c45)cc3c12 ; c1ccc(-c2nc3ccc(-c4cccc5ccc6ccc7c8ccccc8ccc7c6c45)cc3nc2-c2ccccc2)cc1 ; c1ccc(-c2nc3ccccc3nc2-c2cc3c(ccc4ccc5ccccc5c43)c3ccccc23)cc1]

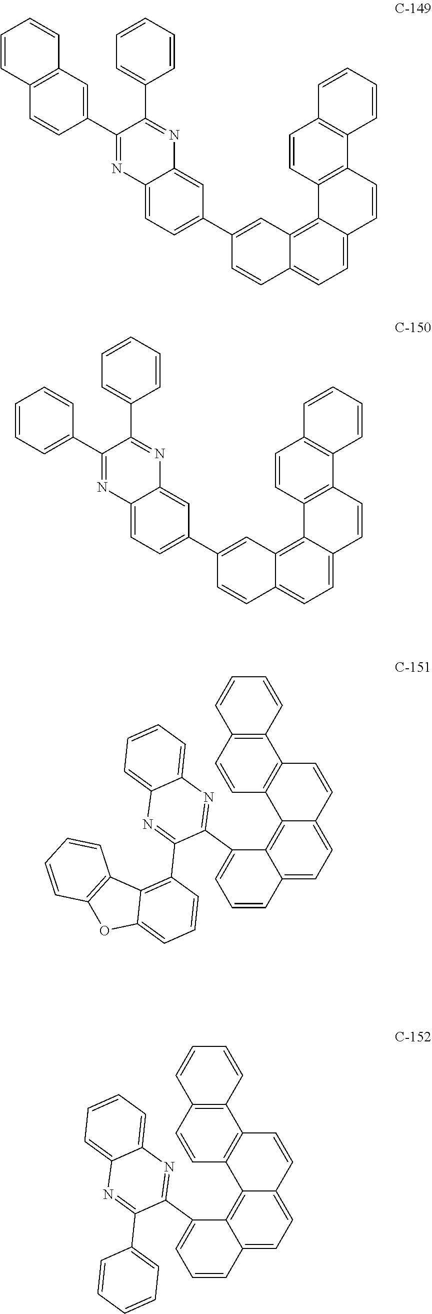 [SMILES: c1ccc(-c2nc3cc(-c4ccc5ccc6ccc7c8ccccc8ccc7c6c5c4)ccc3nc2-c2ccc3ccccc3c2)cc1 ; c1ccc(-c2nc3ccc(-c4ccc5ccc6ccc7c8ccccc8ccc7c6c5c4)cc3nc2-c2ccccc2)cc1 ; c1ccc2c(c1)ccc1c2ccc2ccc3cccc(-c4nc5ccccc5nc4-c4cccc5oc6ccccc6c45)c3c21 ; c1ccc(-c2nc3ccccc3nc2-c2cccc3ccc4ccc5c6ccccc6ccc5c4c23)cc1]

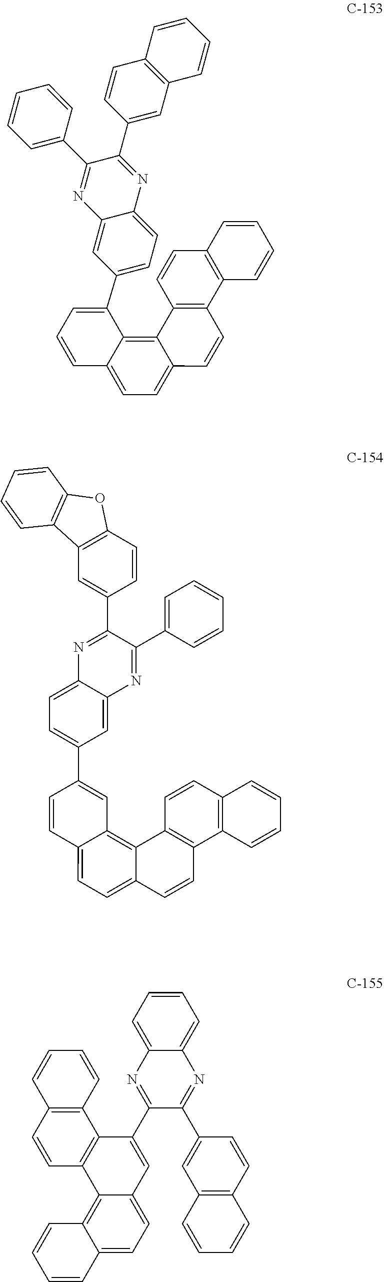 [SMILES: c1ccc(-c2nc3cc(-c4cccc5ccc6ccc7c8ccccc8ccc7c6c45)ccc3nc2-c2ccc3ccccc3c2)cc1 ; c1ccc(-c2nc3cc(-c4ccc5ccc6ccc7c8ccccc8ccc7c6c5c4)ccc3nc2-c2ccc3oc4ccccc4c3c2)cc1 ; c1ccc2cc(-c3nc4ccccc4nc3-c3cc4ccc5ccccc5c4c4ccc5ccccc5c34)ccc2c1]

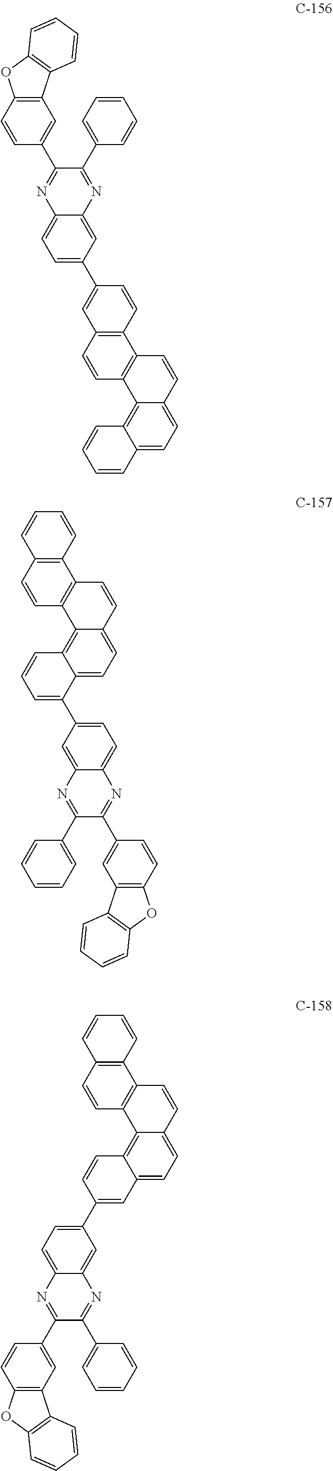 [SMILES: c1ccc(-c2nc3cc(-c4ccc5c(ccc6c5ccc5ccc7ccccc7c56)c4)ccc3nc2-c2ccc3oc4ccccc4c3c2)cc1 ; c1ccc(-c2nc3cc(-c4cccc5c4ccc4ccc6c7ccccc7ccc6c45)ccc3nc2-c2ccc3oc4ccccc4c3c2)cc1 ; c1ccc(-c2nc3cc(-c4ccc5c(ccc6ccc7c8ccccc8ccc7c65)c4)ccc3nc2-c2ccc3oc4ccccc4c3c2)cc1]

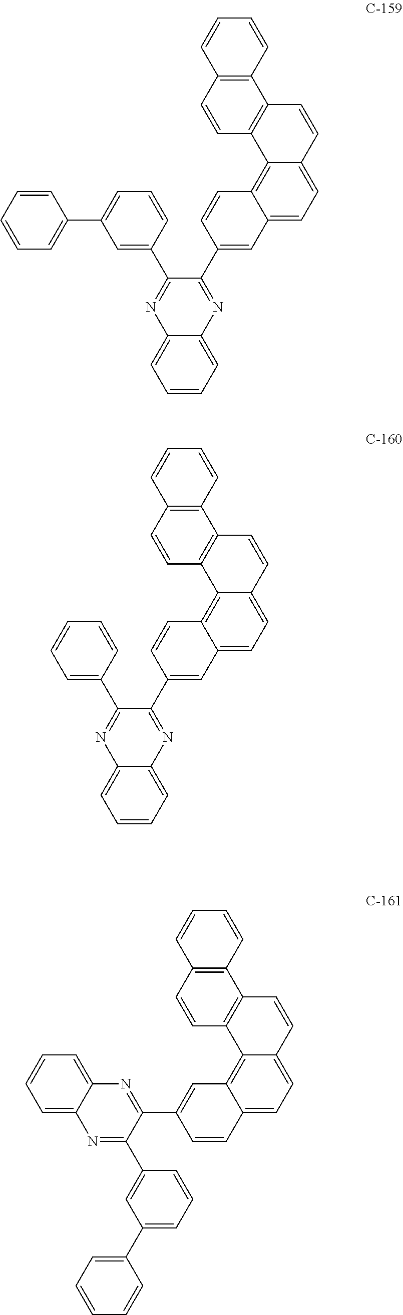 [SMILES: c1ccc(-c2cccc(-c3nc4ccccc4nc3-c3ccc4c(ccc5ccc6c7ccccc7ccc6c54)c3)c2)cc1 ; c1ccc(-c2nc3ccccc3nc2-c2ccc3c(ccc4ccc5c6ccccc6ccc5c43)c2)cc1 ; c1ccc(-c2cccc(-c3nc4ccccc4nc3-c3ccc4ccc5ccc6c7ccccc7ccc6c5c4c3)c2)cc1]

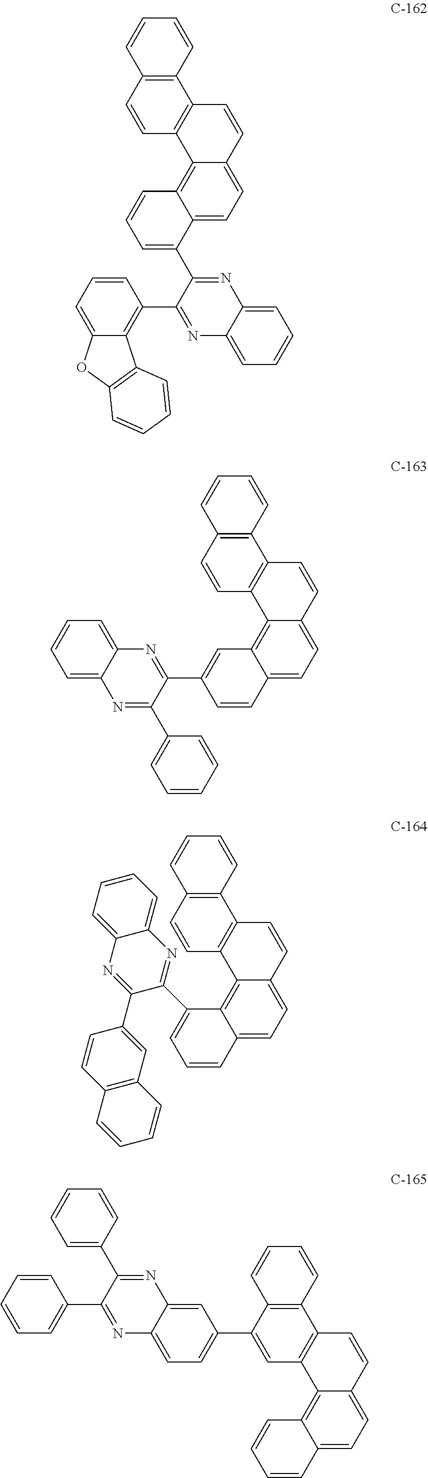

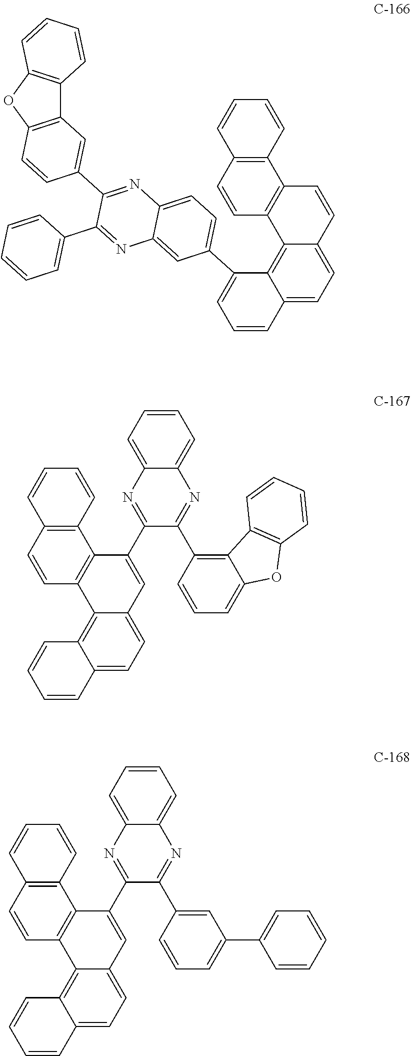 [SMILES: c1ccc(-c2nc3cc(-c4cccc5ccc6ccc7c8ccccc8ccc7c6c45)ccc3nc2-c2ccc3oc4ccccc4c3c2)cc1 ; c1ccc2c(c1)ccc1c2c(-c2nc3ccccc3nc2-c2cccc3oc4ccccc4c23)cc2ccc3ccccc3c21 ; c1ccc(-c2cccc(-c3nc4ccccc4nc3-c3cc4ccc5ccccc5c4c4ccc5ccccc5c34)c2)cc1]

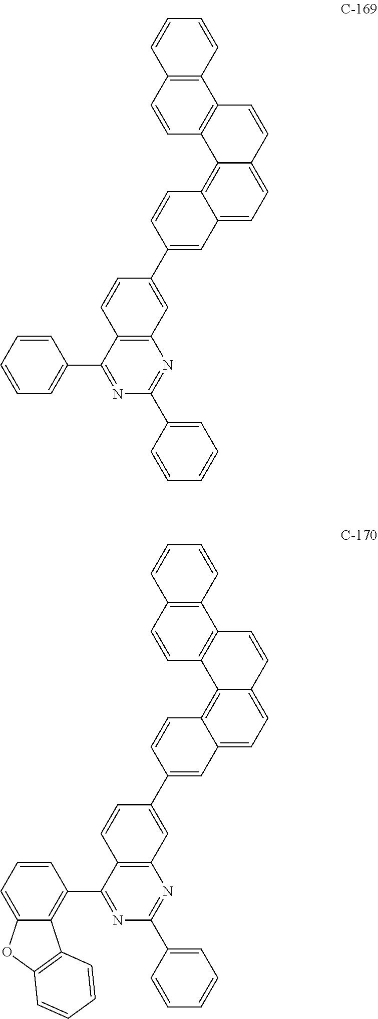 [SMILES: c1ccc(-c2nc(-c3ccccc3)c3ccc(-c4ccc5c(ccc6ccc7c8ccccc8ccc7c65)c4)cc3n2)cc1 ; c1ccc(-c2nc(-c3cccc4oc5ccccc5c34)c3ccc(-c4ccc5c(ccc6ccc7c8ccccc8ccc7c65)c4)cc3n2)cc1]

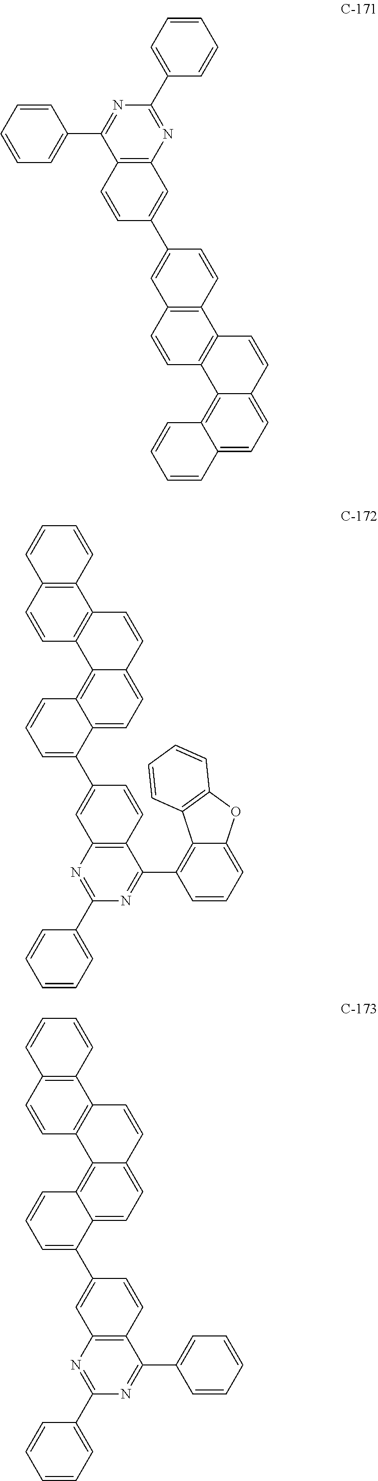

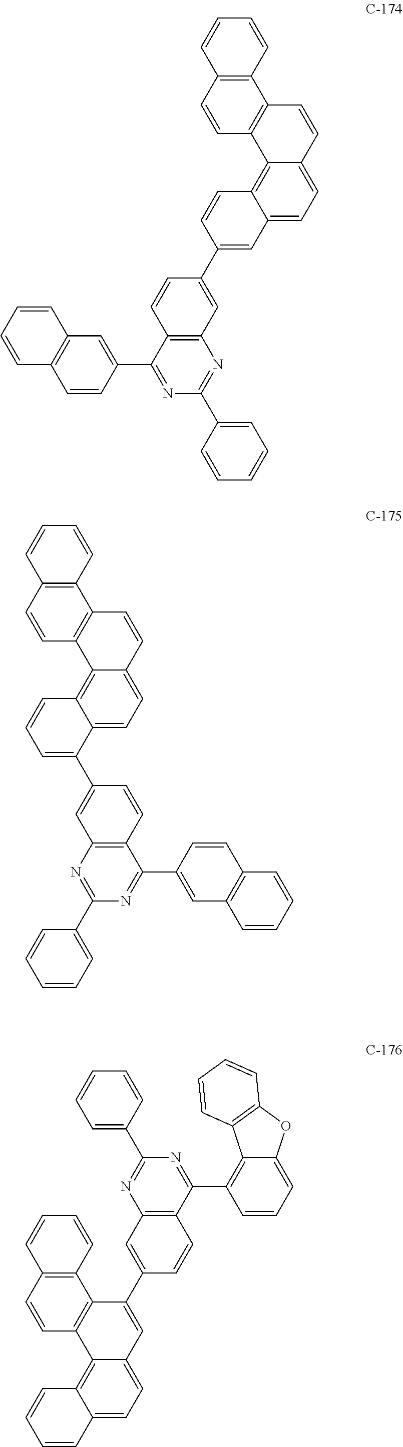 [SMILES: c1ccc(-c2nc(-c3ccc4ccccc4c3)c3ccc(-c4ccc5c(ccc6ccc7c8ccccc8ccc7c65)c4)cc3n2)cc1 ; c1ccc(-c2nc(-c3ccc4ccccc4c3)c3ccc(-c4cccc5c4ccc4ccc6c7ccccc7ccc6c45)cc3n2)cc1 ; c1ccc(-c2nc(-c3cccc4oc5ccccc5c34)c3ccc(-c4cc5ccc6ccccc6c5c5ccc6ccccc6c45)cc3n2)cc1]

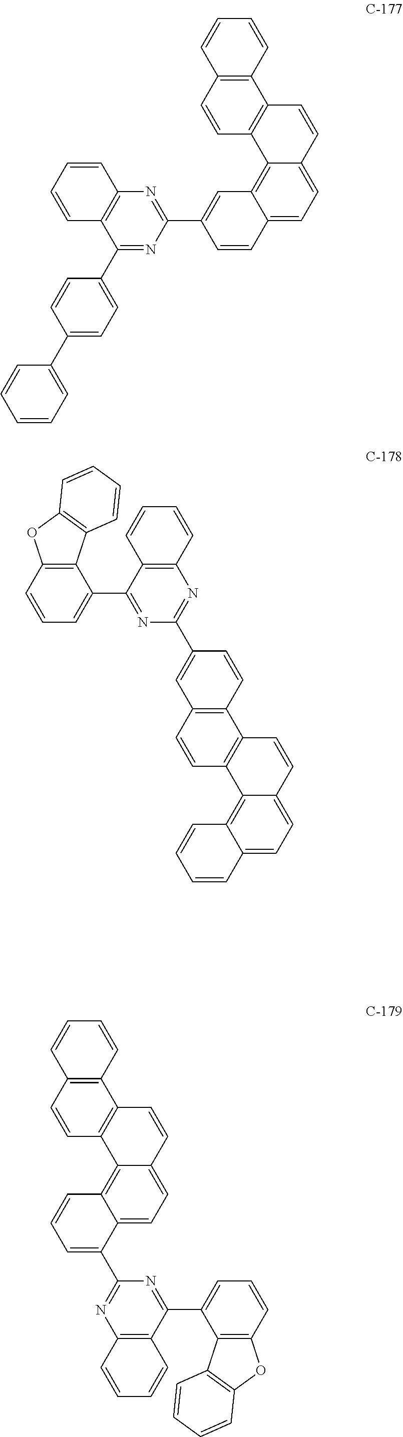 [SMILES: c1ccc(-c2ccc(-c3nc(-c4ccc5ccc6ccc7c8ccccc8ccc7c6c5c4)nc4ccccc34)cc2)cc1 ; c1ccc2c(c1)ccc1ccc3c4ccc(-c5nc(-c6cccc7oc8ccccc8c67)c6ccccc6n5)cc4ccc3c12 ; c1ccc2c(c1)ccc1c2ccc2ccc3c(-c4nc(-c5cccc6oc7ccccc7c56)c5ccccc5n4)cccc3c21]

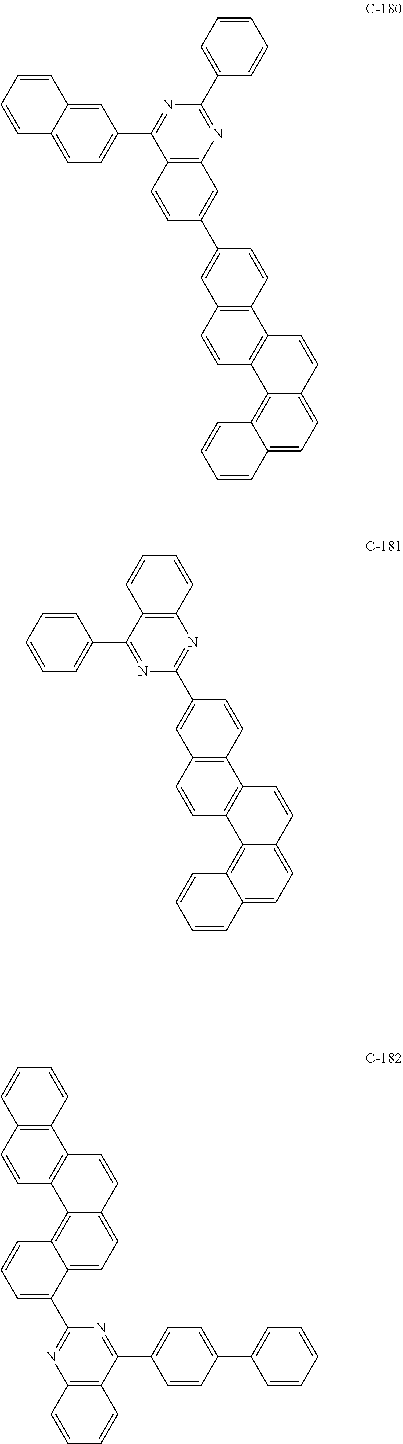 [SMILES: c1ccc(-c2nc(-c3ccc4ccccc4c3)c3ccc(-c4ccc5c(ccc6c5ccc5ccc7ccccc7c56)c4)cc3n2)cc1 ; c1ccc(-c2nc(-c3ccc4c(ccc5c4ccc4ccc6ccccc6c45)c3)nc3ccccc23)cc1 ; c1ccc(-c2ccc(-c3nc(-c4cccc5c4ccc4ccc6c7ccccc7ccc6c45)nc4ccccc34)cc2)cc1]

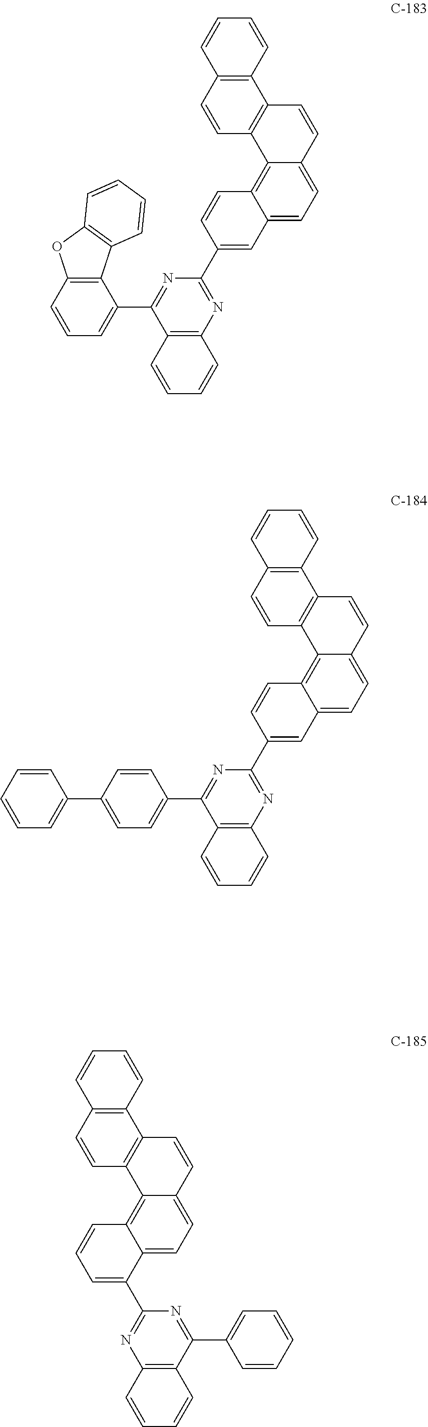 [SMILES: c1ccc2c(c1)ccc1c2ccc2ccc3cc(-c4nc(-c5cccc6oc7ccccc7c56)c5ccccc5n4)ccc3c21 ; c1ccc(-c2ccc(-c3nc(-c4ccc5c(ccc6ccc7c8ccccc8ccc7c65)c4)nc4ccccc34)cc2)cc1 ; c1ccc(-c2nc(-c3cccc4c3ccc3ccc5c6ccccc6ccc5c34)nc3ccccc23)cc1]

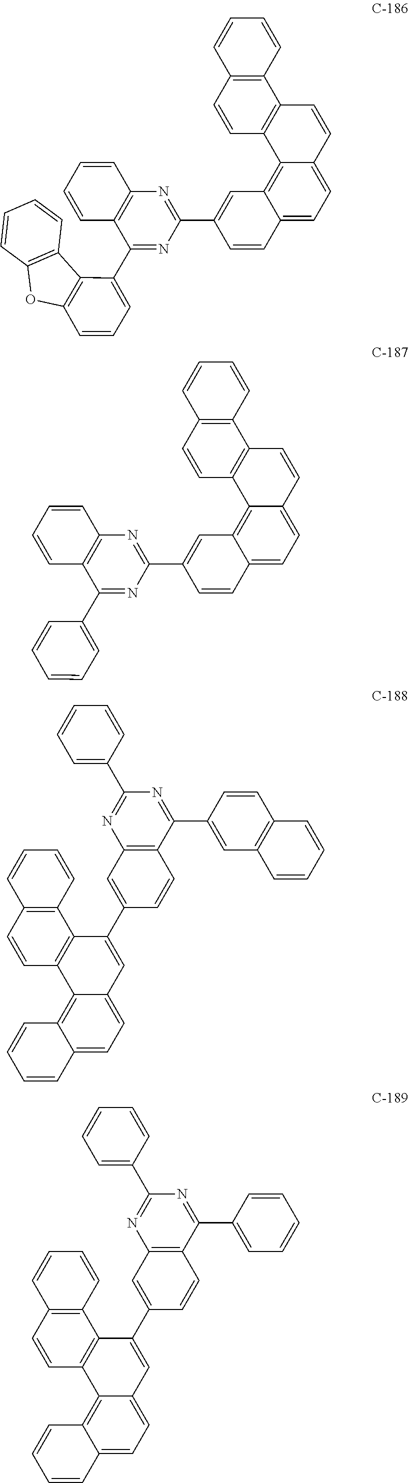

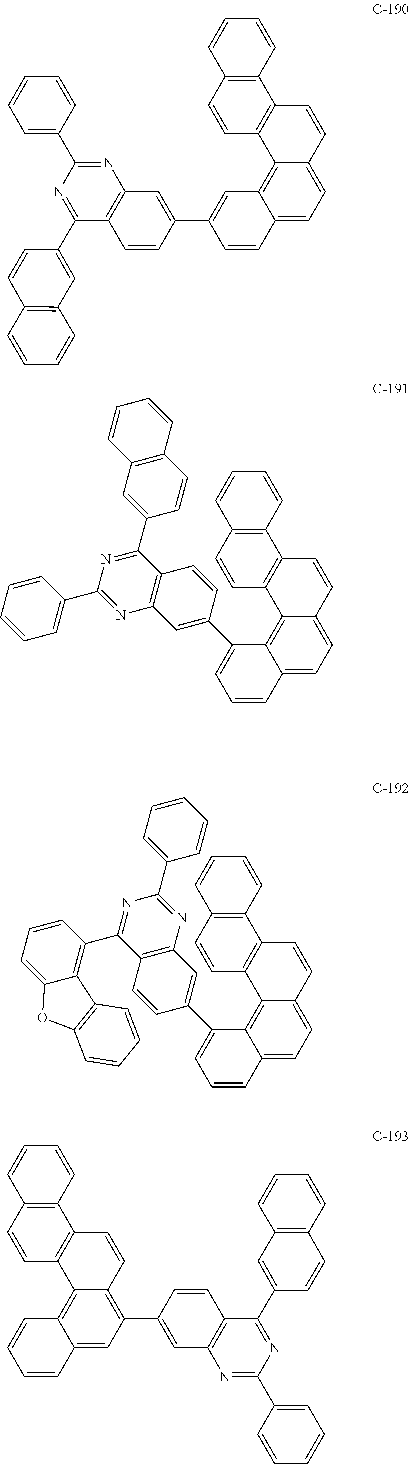 [SMILES: c1ccc(-c2nc(-c3ccc4ccccc4c3)c3ccc(-c4ccc5ccc6ccc7c8ccccc8ccc7c6c5c4)cc3n2)cc1 ; c1ccc(-c2nc(-c3ccc4ccccc4c3)c3ccc(-c4cccc5ccc6ccc7c8ccccc8ccc7c6c45)cc3n2)cc1 ; c1ccc(-c2nc(-c3cccc4oc5ccccc5c34)c3ccc(-c4cccc5ccc6ccc7c8ccccc8ccc7c6c45)cc3n2)cc1 ; c1ccc(-c2nc(-c3ccc4ccccc4c3)c3ccc(-c4cc5ccccc5c5c4ccc4c6ccccc6ccc45)cc3n2)cc1]

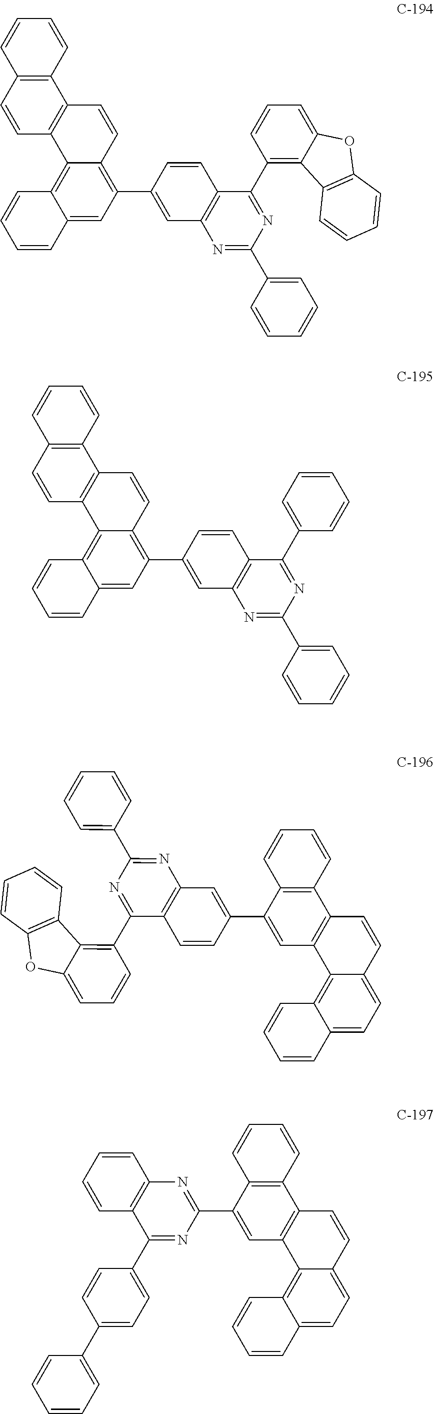 [SMILES: c1ccc(-c2nc(-c3cccc4oc5ccccc5c34)c3ccc(-c4cc5ccccc5c5c4ccc4c6ccccc6ccc45)cc3n2)cc1 ; c1ccc(-c2nc(-c3ccccc3)c3ccc(-c4cc5ccccc5c5c4ccc4c6ccccc6ccc45)cc3n2)cc1 ; c1ccc(-c2nc(-c3cccc4oc5ccccc5c34)c3ccc(-c4cc5c(ccc6ccc7ccccc7c65)c5ccccc45)cc3n2)cc1 ; c1ccc(-c2ccc(-c3nc(-c4cc5c(ccc6ccc7ccccc7c65)c5ccccc45)nc4ccccc34)cc2)cc1]

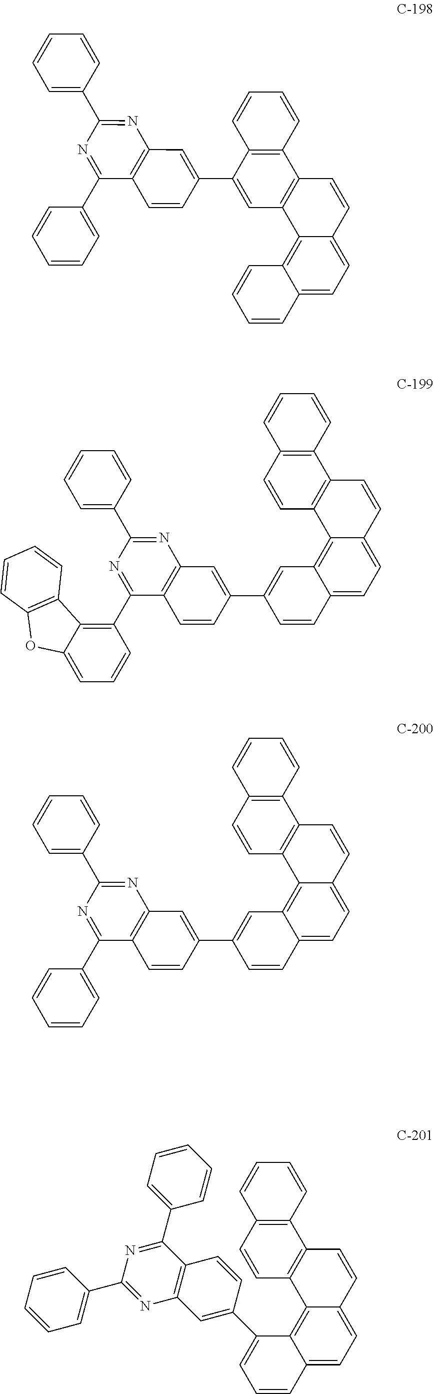 [SMILES: c1ccc(-c2nc(-c3ccccc3)c3ccc(-c4cc5c(ccc6ccc7ccccc7c65)c5ccccc45)cc3n2)cc1 ; c1ccc(-c2nc(-c3cccc4oc5ccccc5c34)c3ccc(-c4ccc5ccc6ccc7c8ccccc8ccc7c6c5c4)cc3n2)cc1 ; c1ccc(-c2nc(-c3ccccc3)c3ccc(-c4ccc5ccc6ccc7c8ccccc8ccc7c6c5c4)cc3n2)cc1 ; c1ccc(-c2nc(-c3ccccc3)c3ccc(-c4cccc5ccc6ccc7c8ccccc8ccc7c6c45)cc3n2)cc1]

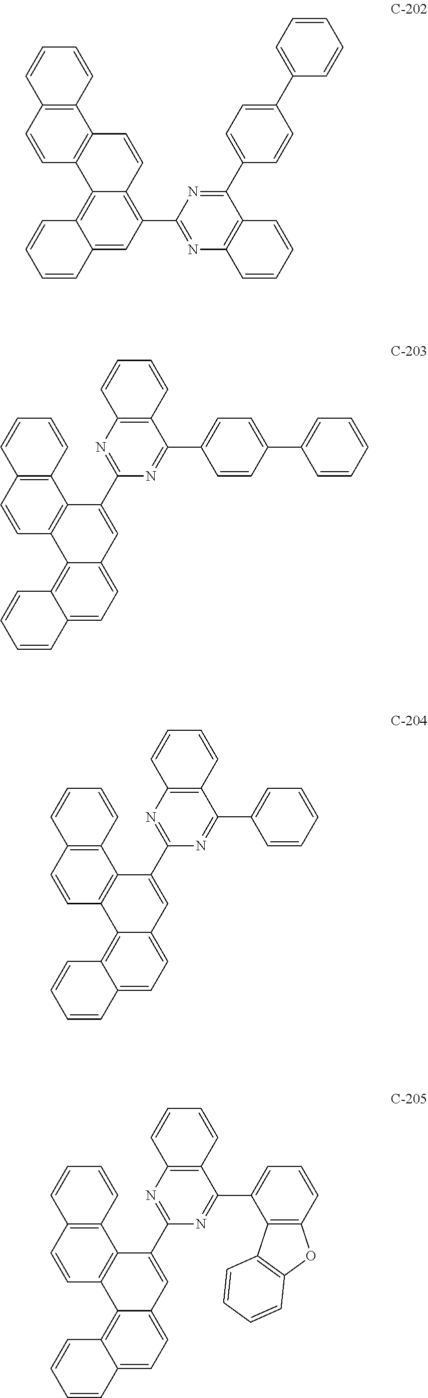 [SMILES: c1ccc(-c2ccc(-c3nc(-c4cc5ccccc5c5c4ccc4c6ccccc6ccc45)nc4ccccc34)cc2)cc1 ; c1ccc(-c2ccc(-c3nc(-c4cc5ccc6ccccc6c5c5ccc6ccccc6c45)nc4ccccc34)cc2)cc1 ; c1ccc(-c2nc(-c3cc4ccc5ccccc5c4c4ccc5ccccc5c34)nc3ccccc23)cc1 ; c1ccc2c(c1)ccc1c2c(-c2nc(-c3cccc4oc5ccccc5c34)c3ccccc3n2)cc2ccc3ccccc3c21]

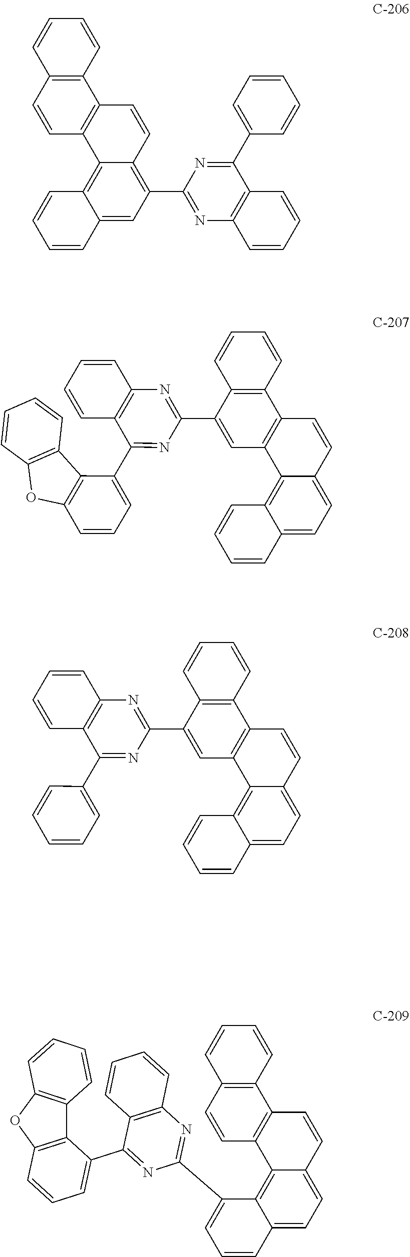 [SMILES: c1ccc(-c2nc(-c3cc4ccccc4c4c3ccc3c5ccccc5ccc34)nc3ccccc23)cc1 ; c1ccc2c(c1)ccc1ccc3c4ccccc4c(-c4nc(-c5cccc6oc7ccccc7c56)c5ccccc5n4)cc3c12 ; c1ccc(-c2nc(-c3cc4c(ccc5ccc6ccccc6c54)c4ccccc34)nc3ccccc23)cc1 ; c1ccc2c(c1)ccc1c2ccc2ccc3cccc(-c4nc(-c5cccc6oc7ccccc7c56)c5ccccc5n4)c3c21]

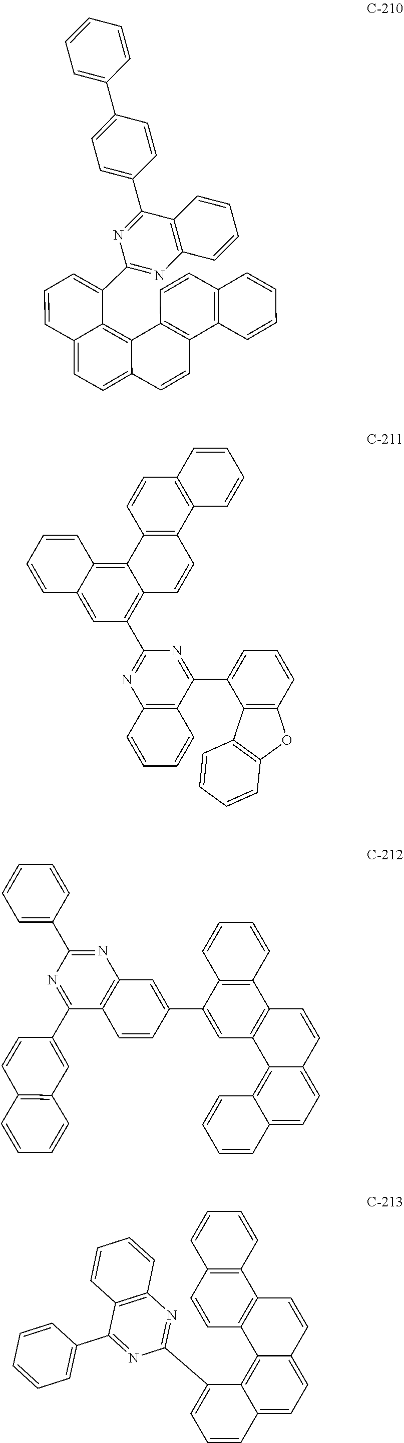 [SMILES: c1ccc(-c2ccc(-c3nc(-c4cccc5ccc6ccc7c8ccccc8ccc7c6c45)nc4ccccc34)cc2)cc1 ; c1ccc2c(c1)ccc1c2ccc2c(-c3nc(-c4cccc5oc6ccccc6c45)c4ccccc4n3)cc3ccccc3c21 ; c1ccc(-c2nc(-c3ccc4ccccc4c3)c3ccc(-c4cc5c(ccc6ccc7ccccc7c65)c5ccccc45)cc3n2)cc1 ; c1ccc(-c2nc(-c3cccc4ccc5ccc6c7ccccc7ccc6c5c34)nc3ccccc23)cc1]

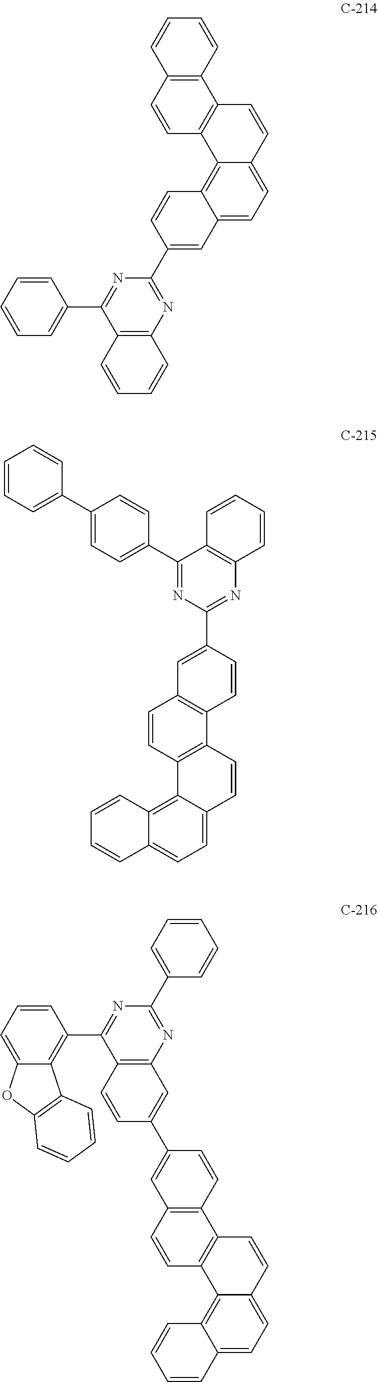 [SMILES: c1ccc(-c2nc(-c3ccc4c(ccc5ccc6c7ccccc7ccc6c54)c3)nc3ccccc23)cc1 ; c1ccc(-c2ccc(-c3nc(-c4ccc5c(ccc6c5ccc5ccc7ccccc7c56)c4)nc4ccccc34)cc2)cc1 ; c1ccc(-c2nc(-c3cccc4oc5ccccc5c34)c3ccc(-c4ccc5c(ccc6c5ccc5ccc7ccccc7c56)c4)cc3n2)cc1]

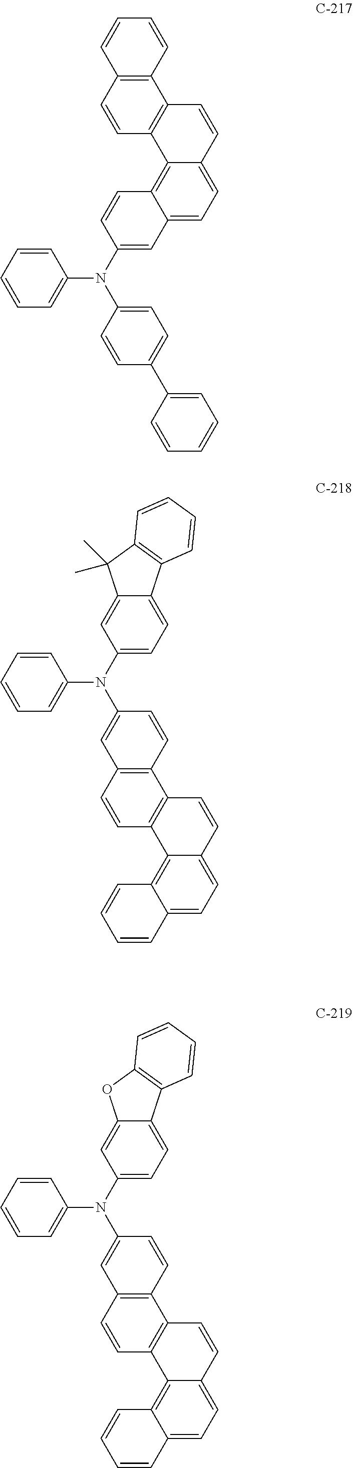

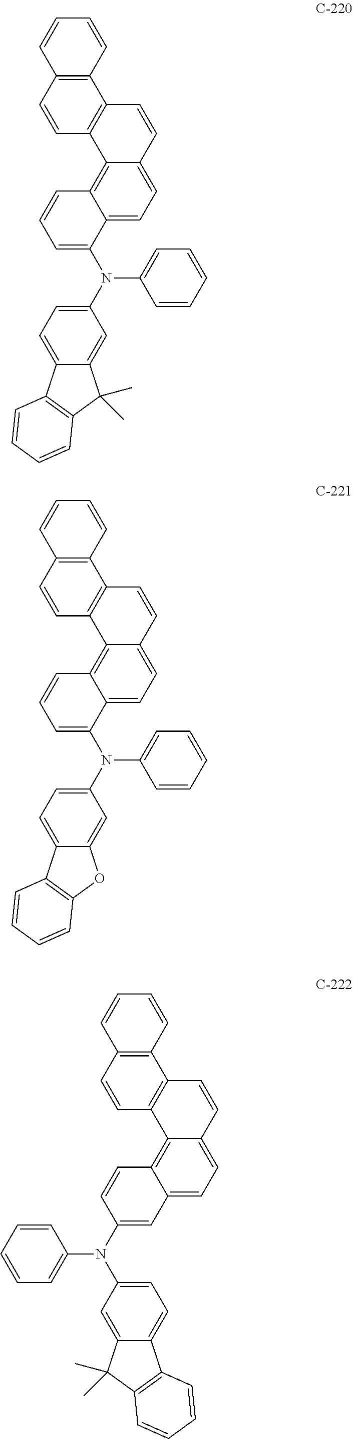

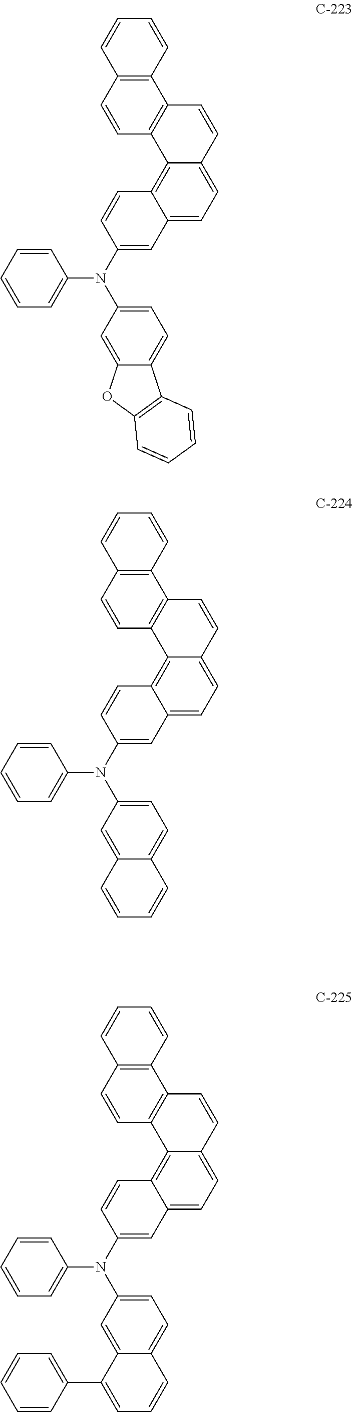 [SMILES: c1ccc(N(c2ccc3c(ccc4ccc5c6ccccc6ccc5c43)c2)c2ccc3c(c2)oc2ccccc23)cc1 ; c1ccc(N(c2ccc3ccccc3c2)c2ccc3c(ccc4ccc5c6ccccc6ccc5c43)c2)cc1 ; c1ccc(-c2cccc3ccc(N(c4ccccc4)c4ccc5c(ccc6ccc7c8ccccc8ccc7c65)c4)cc23)cc1]

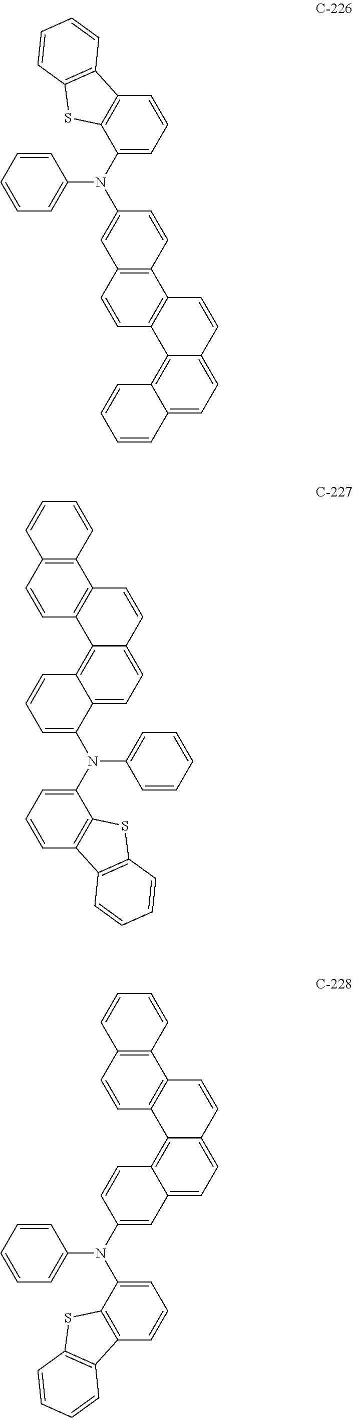 [SMILES: c1ccc(N(c2ccc3c(ccc4c3ccc3ccc5ccccc5c34)c2)c2cccc3c2sc2ccccc23)cc1 ; c1ccc(N(c2cccc3c2ccc2ccc4c5ccccc5ccc4c23)c2cccc3c2sc2ccccc23)cc1 ; c1ccc(N(c2ccc3c(ccc4ccc5c6ccccc6ccc5c43)c2)c2cccc3c2sc2ccccc23)cc1]

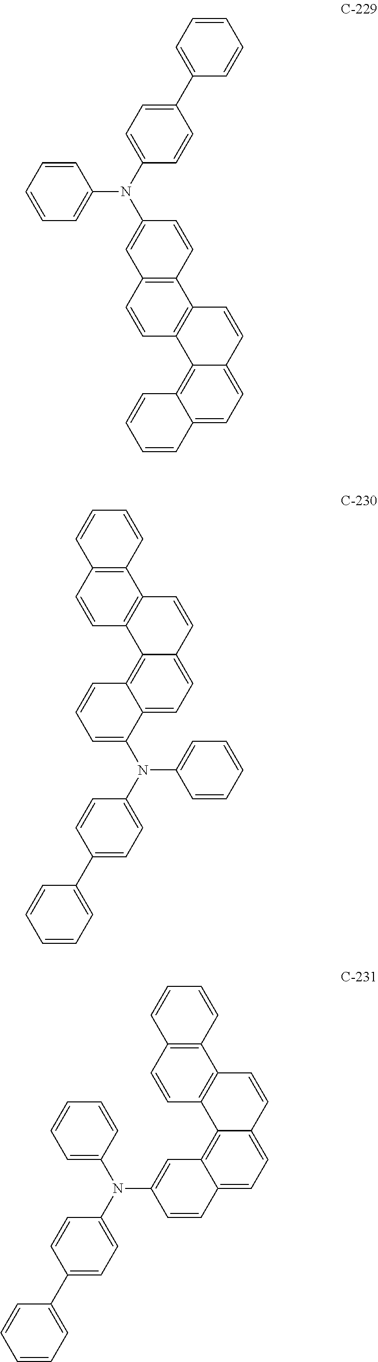 [SMILES: c1ccc(-c2ccc(N(c3ccccc3)c3ccc4c(ccc5c4ccc4ccc6ccccc6c45)c3)cc2)cc1 ; c1ccc(-c2ccc(N(c3ccccc3)c3cccc4c3ccc3ccc5c6ccccc6ccc5c34)cc2)cc1 ; c1ccc(-c2ccc(N(c3ccccc3)c3ccc4ccc5ccc6c7ccccc7ccc6c5c4c3)cc2)cc1]

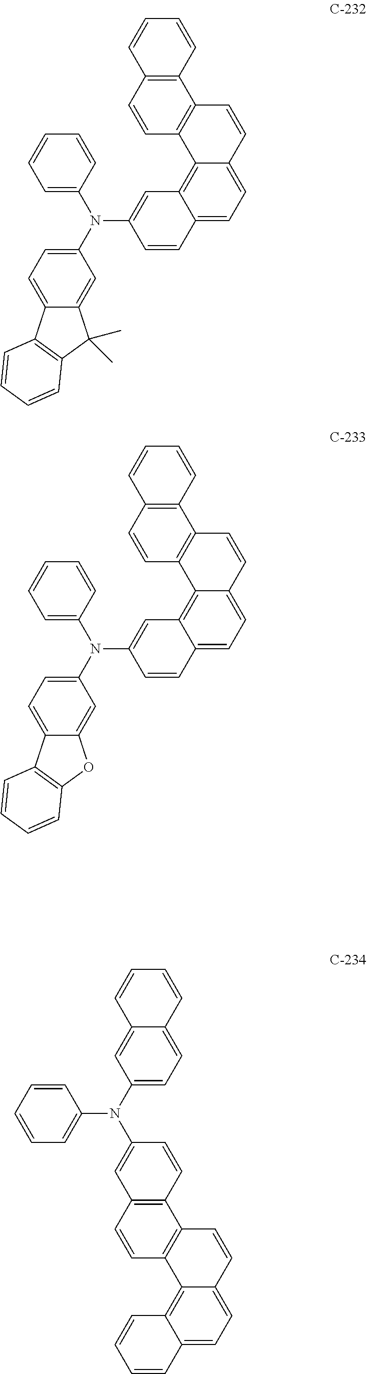 [SMILES: CC1(C)c2ccccc2-c2ccc(N(c3ccccc3)c3ccc4ccc5ccc6c7ccccc7ccc6c5c4c3)cc21 ; c1ccc(N(c2ccc3c(c2)oc2ccccc23)c2ccc3ccc4ccc5c6ccccc6ccc5c4c3c2)cc1 ; c1ccc(N(c2ccc3ccccc3c2)c2ccc3c(ccc4c3ccc3ccc5ccccc5c34)c2)cc1]

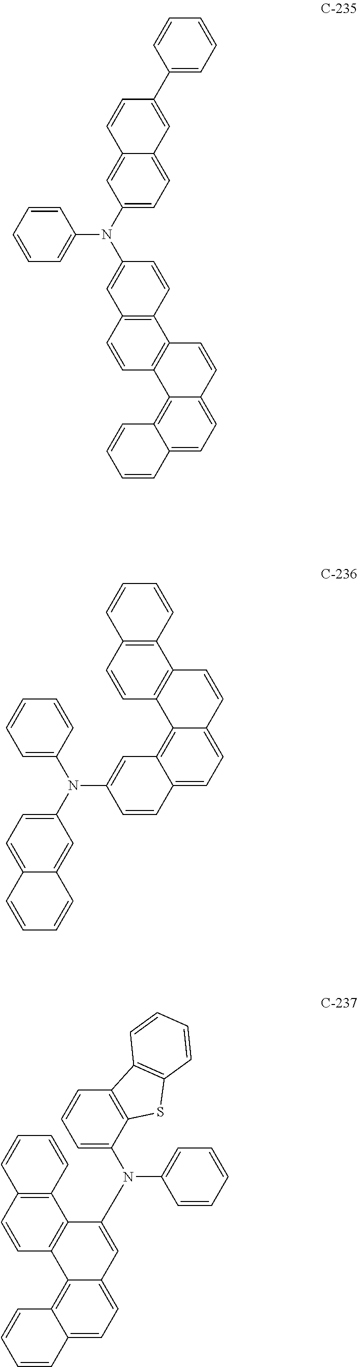 [SMILES: c1ccc(-c2ccc3cc(N(c4ccccc4)c4ccc5c(ccc6c5ccc5ccc7ccccc7c56)c4)ccc3c2)cc1 ; c1ccc(N(c2ccc3ccccc3c2)c2ccc3ccc4ccc5c6ccccc6ccc5c4c3c2)cc1 ; c1ccc(N(c2cccc3c2sc2ccccc23)c2cc3ccc4ccccc4c3c3ccc4ccccc4c23)cc1]

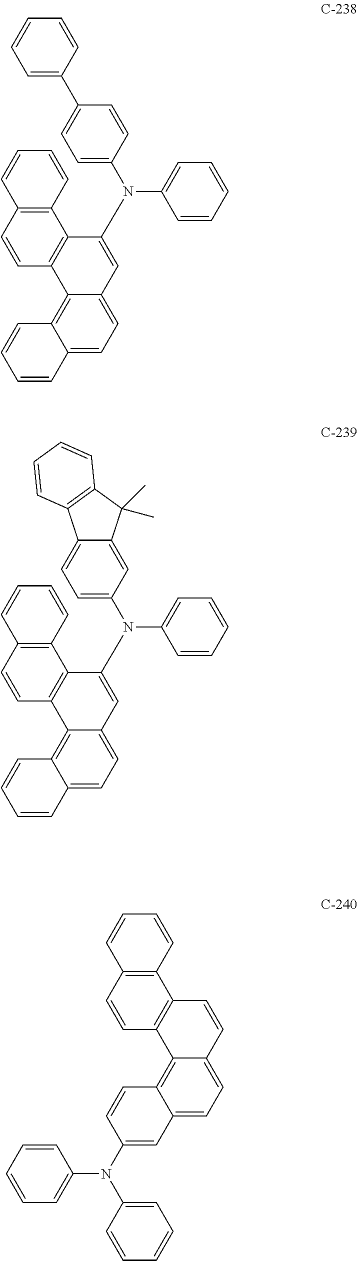 [SMILES: c1ccc(-c2ccc(N(c3ccccc3)c3cc4ccc5ccccc5c4c4ccc5ccccc5c34)cc2)cc1 ; CC1(C)c2ccccc2-c2ccc(N(c3ccccc3)c3cc4ccc5ccccc5c4c4ccc5ccccc5c34)cc21 ; c1ccc(N(c2ccccc2)c2ccc3c(ccc4ccc5c6ccccc6ccc5c43)c2)cc1]

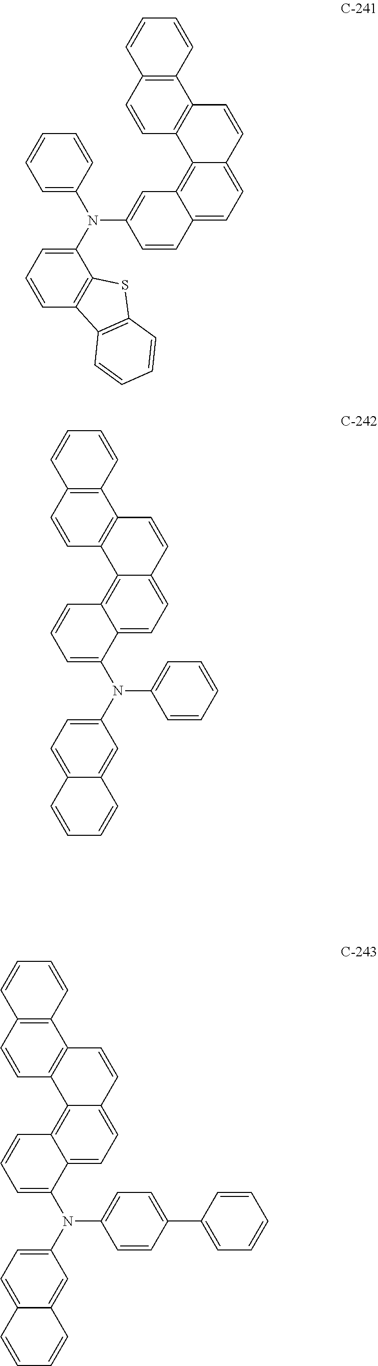 [SMILES: c1ccc(N(c2ccc3ccc4ccc5c6ccccc6ccc5c4c3c2)c2cccc3c2sc2ccccc23)cc1 ; c1ccc(N(c2ccc3ccccc3c2)c2cccc3c2ccc2ccc4c5ccccc5ccc4c23)cc1 ; c1ccc(-c2ccc(N(c3ccc4ccccc4c3)c3cccc4c3ccc3ccc5c6ccccc6ccc5c34)cc2)cc1]

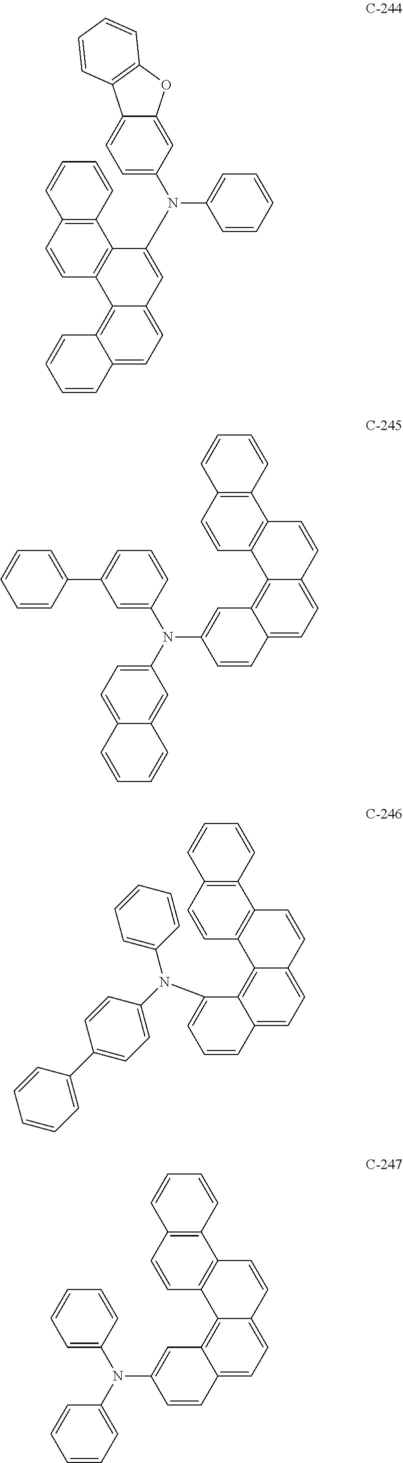 [SMILES: c1ccc(N(c2ccc3c(c2)oc2ccccc23)c2cc3ccc4ccccc4c3c3ccc4ccccc4c23)cc1 ; c1ccc(-c2cccc(N(c3ccc4ccccc4c3)c3ccc4ccc5ccc6c7ccccc7ccc6c5c4c3)c2)cc1 ; c1ccc(-c2ccc(N(c3ccccc3)c3cccc4ccc5ccc6c7ccccc7ccc6c5c34)cc2)cc1 ; c1ccc(N(c2ccccc2)c2ccc3ccc4ccc5c6ccccc6ccc5c4c3c2)cc1]

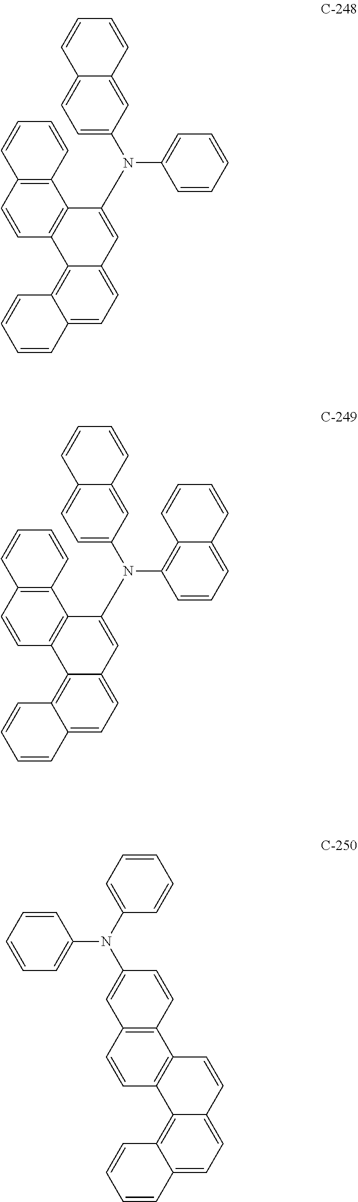 [SMILES: c1ccc(N(c2ccc3ccccc3c2)c2cc3ccc4ccccc4c3c3ccc4ccccc4c23)cc1 ; c1ccc2cc(N(c3cccc4ccccc34)c3cc4ccc5ccccc5c4c4ccc5ccccc5c34)ccc2c1 ; c1ccc(N(c2ccccc2)c2ccc3c(ccc4c3ccc3ccc5ccccc5c34)c2)cc1]

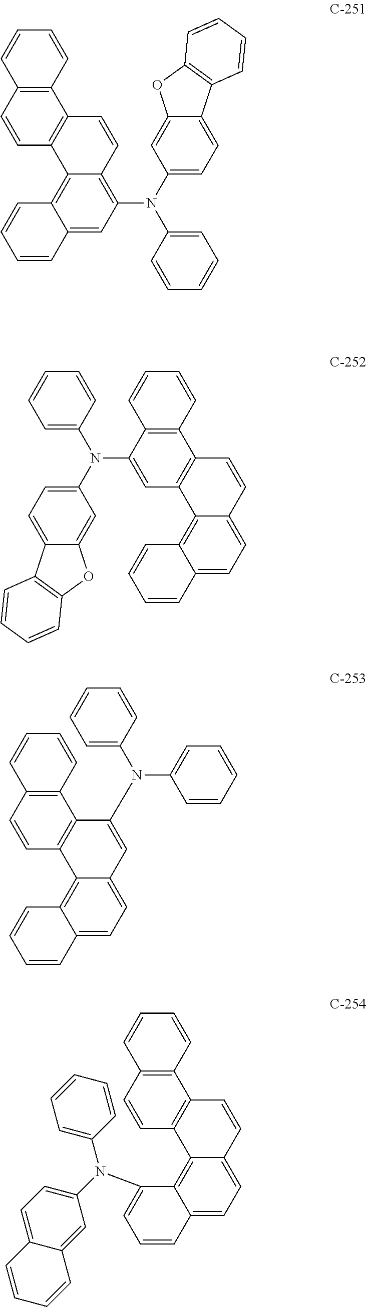 [SMILES: c1ccc(N(c2ccc3c(c2)oc2ccccc23)c2cc3ccccc3c3c2ccc2c4ccccc4ccc23)cc1 ; c1ccc(N(c2ccc3c(c2)oc2ccccc23)c2cc3c(ccc4ccc5ccccc5c43)c3ccccc23)cc1 ; c1ccc(N(c2ccccc2)c2cc3ccc4ccccc4c3c3ccc4ccccc4c23)cc1 ; c1ccc(N(c2ccc3ccccc3c2)c2cccc3ccc4ccc5c6ccccc6ccc5c4c23)cc1]

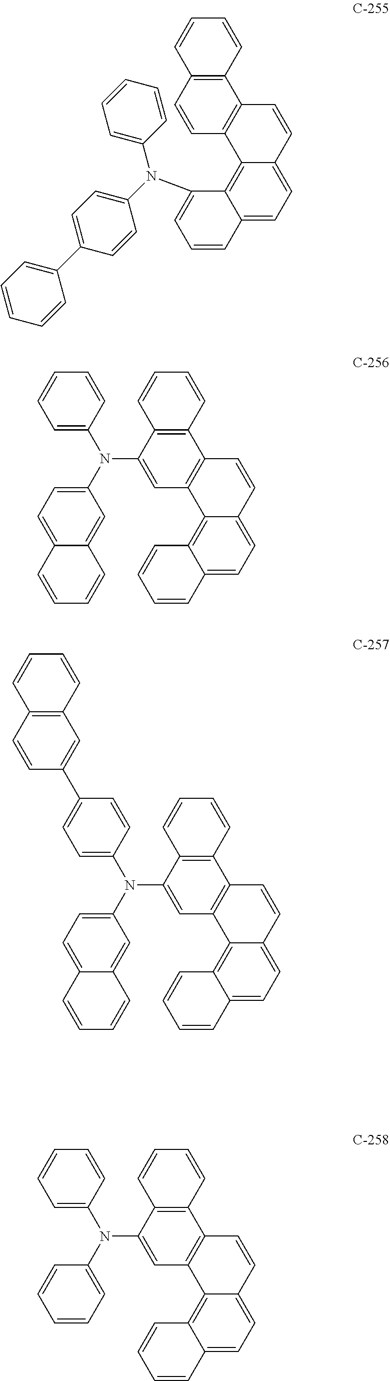 [SMILES: c1ccc(-c2ccc(N(c3ccccc3)c3cccc4ccc5ccc6c7ccccc7ccc6c5c34)cc2)cc1 ; c1ccc(N(c2ccc3ccccc3c2)c2cc3c(ccc4ccc5ccccc5c43)c3ccccc23)cc1 ; c1ccc2cc(-c3ccc(N(c4ccc5ccccc5c4)c4cc5c(ccc6ccc7ccccc7c65)c5ccccc45)cc3)ccc2c1 ; c1ccc(N(c2ccccc2)c2cc3c(ccc4ccc5ccccc5c43)c3ccccc23)cc1]

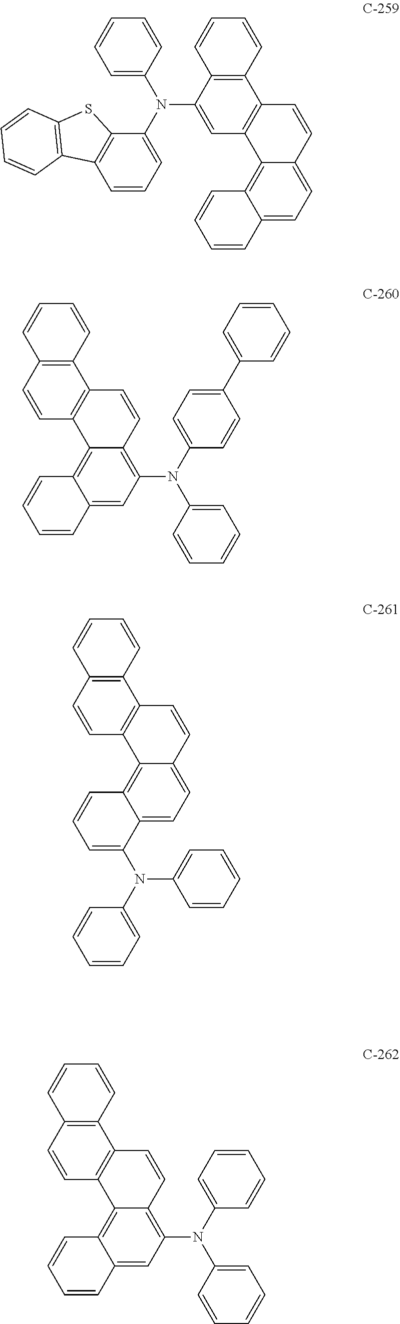 [SMILES: c1ccc(N(c2cc3c(ccc4ccc5ccccc5c43)c3ccccc23)c2cccc3c2sc2ccccc23)cc1 ; c1ccc(-c2ccc(N(c3ccccc3)c3cc4ccccc4c4c3ccc3c5ccccc5ccc34)cc2)cc1 ; c1ccc(N(c2ccccc2)c2cccc3c2ccc2ccc4c5ccccc5ccc4c23)cc1 ; c1ccc(N(c2ccccc2)c2cc3ccccc3c3c2ccc2c4ccccc4ccc23)cc1]

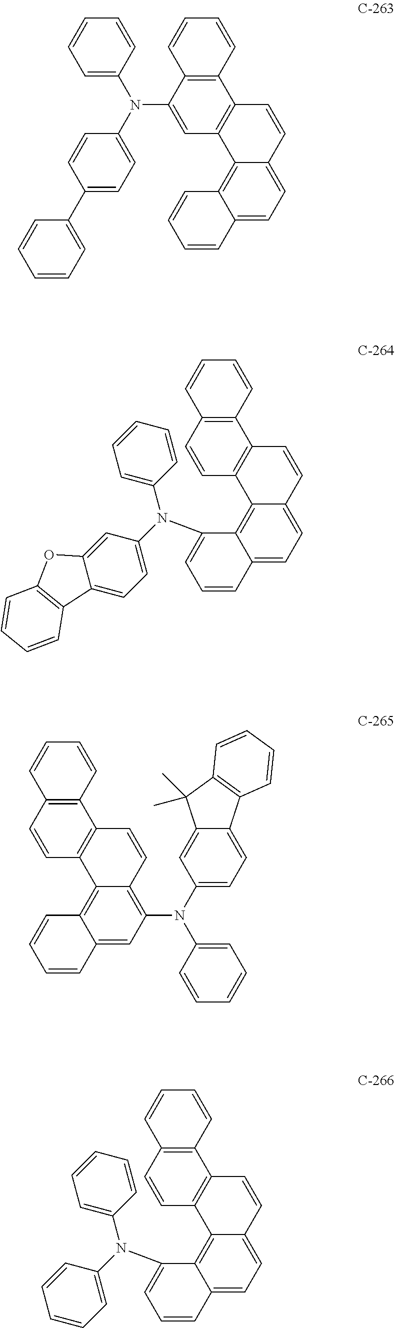 [SMILES: c1ccc(-c2ccc(N(c3ccccc3)c3cc4c(ccc5ccc6ccccc6c54)c4ccccc34)cc2)cc1 ; c1ccc(N(c2ccc3c(c2)oc2ccccc23)c2cccc3ccc4ccc5c6ccccc6ccc5c4c23)cc1 ; CC1(C)c2ccccc2-c2ccc(N(c3ccccc3)c3cc4ccccc4c4c3ccc3c5ccccc5ccc34)cc21 ; c1ccc(N(c2ccccc2)c2cccc3ccc4ccc5c6ccccc6ccc5c4c23)cc1]

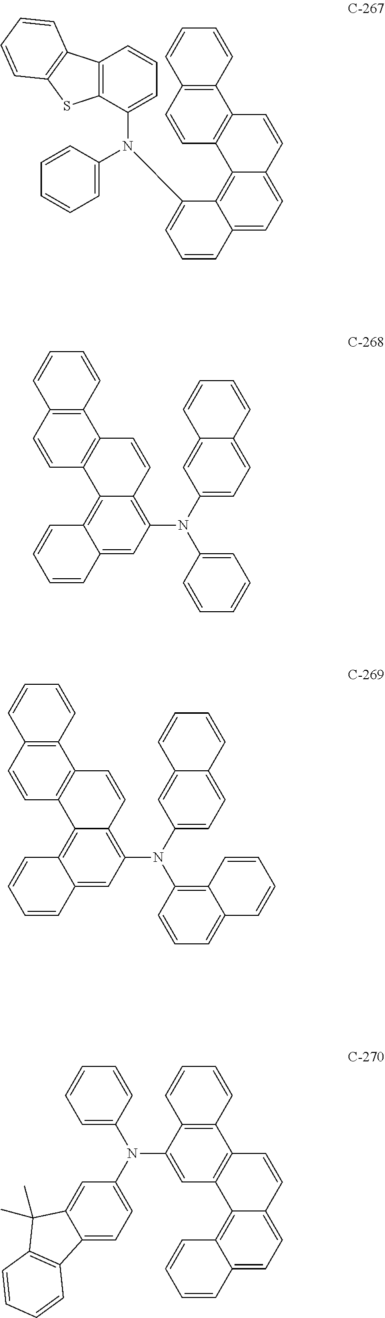 [SMILES: c1ccc(N(c2cccc3c2sc2ccccc23)c2cccc3ccc4ccc5c6ccccc6ccc5c4c23)cc1 ; c1ccc(N(c2ccc3ccccc3c2)c2cc3ccccc3c3c2ccc2c4ccccc4ccc23)cc1 ; c1ccc2cc(N(c3cccc4ccccc34)c3cc4ccccc4c4c3ccc3c5ccccc5ccc34)ccc2c1 ; CC1(C)c2ccccc2-c2ccc(N(c3ccccc3)c3cc4c(ccc5ccc6ccccc6c54)c4ccccc34)cc21]

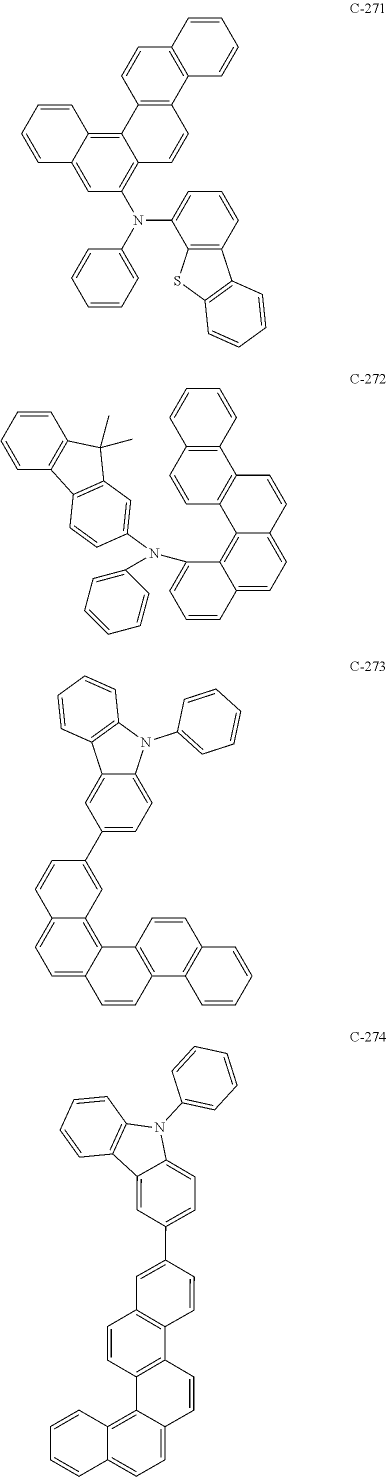

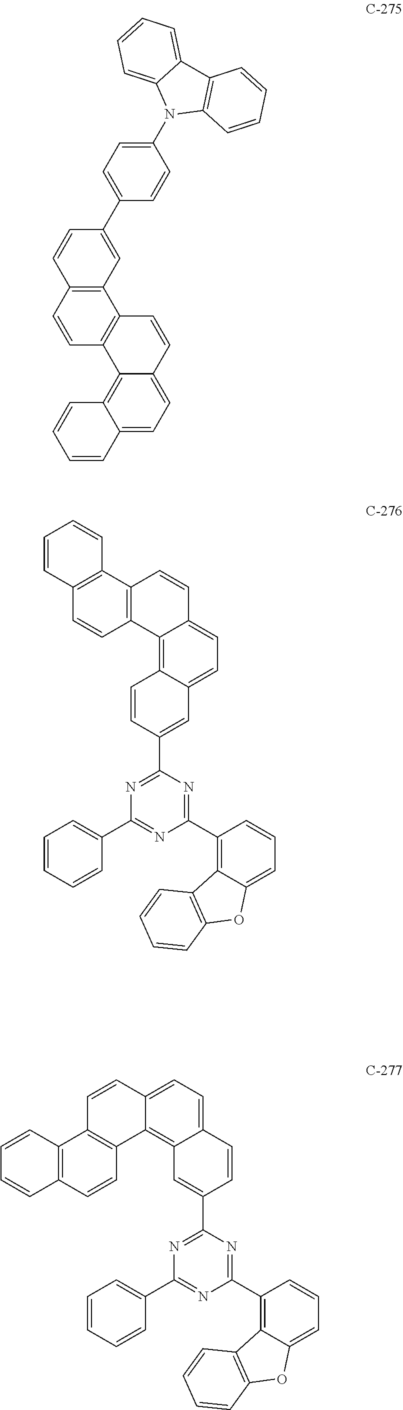 [SMILES: c1ccc2c(c1)ccc1ccc3c4cc(-c5ccc(-n6c7ccccc7c7ccccc76)cc5)ccc4ccc3c12 ; c1ccc(-c2nc(-c3ccc4c(ccc5ccc6c7ccccc7ccc6c54)c3)nc(-c3cccc4oc5ccccc5c34)n2)cc1 ; c1ccc(-c2nc(-c3ccc4ccc5ccc6c7ccccc7ccc6c5c4c3)nc(-c3cccc4oc5ccccc5c34)n2)cc1]

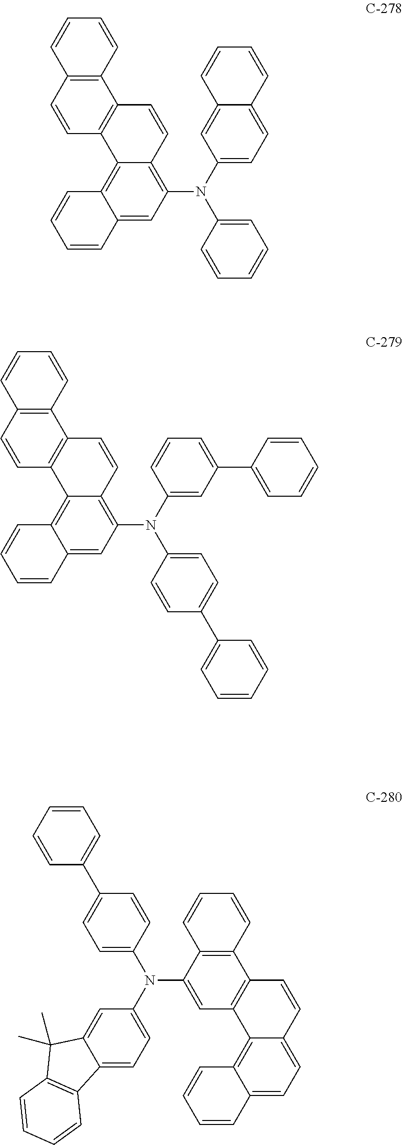 [SMILES: c1ccc(N(c2ccc3ccccc3c2)c2cc3ccccc3c3c2ccc2c4ccccc4ccc23)cc1 ; c1ccc(-c2ccc(N(c3cccc(-c4ccccc4)c3)c3cc4ccccc4c4c3ccc3c5ccccc5ccc34)cc2)cc1 ; CC1(C)c2ccccc2-c2ccc(N(c3ccc(-c4ccccc4)cc3)c3cc4c(ccc5ccc6ccccc6c54)c4ccccc34)cc21]

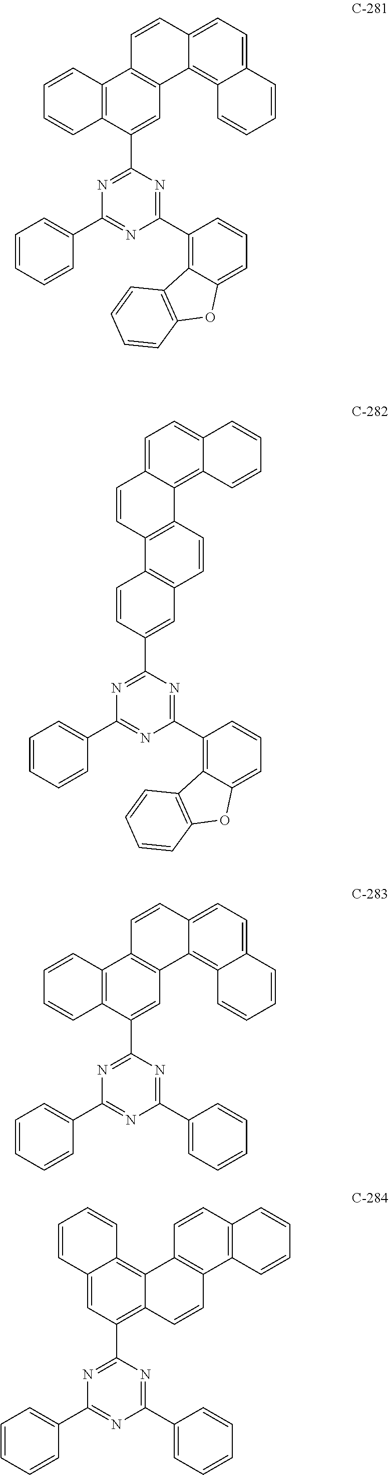

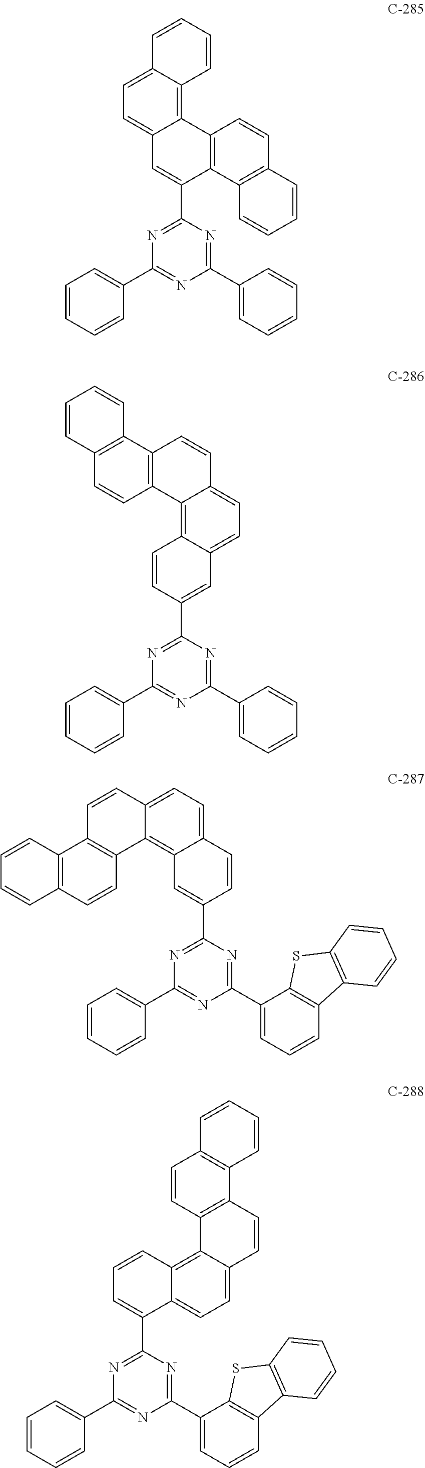

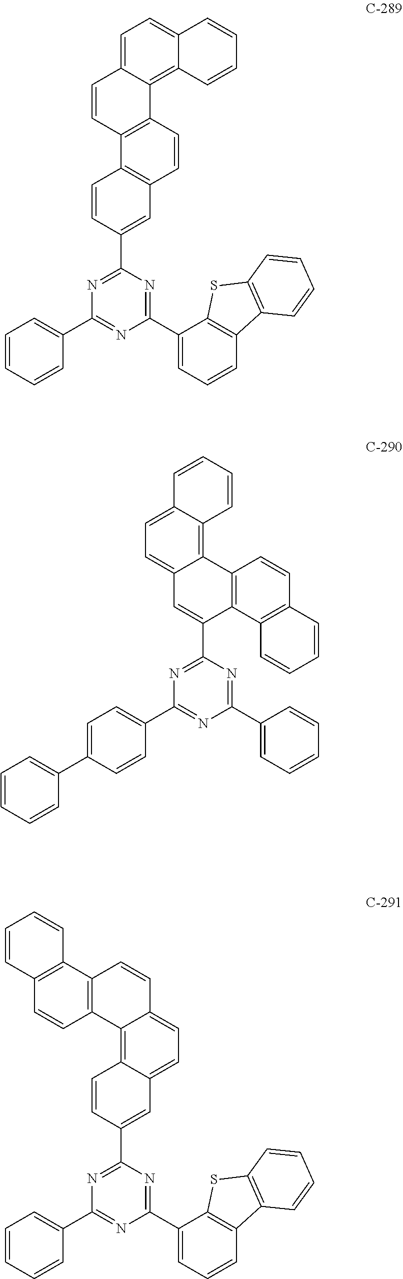 [SMILES: c1ccc(-c2nc(-c3ccc4c(ccc5c4ccc4ccc6ccccc6c45)c3)nc(-c3cccc4c3sc3ccccc34)n2)cc1 ; c1ccc(-c2ccc(-c3nc(-c4ccccc4)nc(-c4cc5ccc6ccccc6c5c5ccc6ccccc6c45)n3)cc2)cc1 ; c1ccc(-c2nc(-c3ccc4c(ccc5ccc6c7ccccc7ccc6c54)c3)nc(-c3cccc4c3sc3ccccc34)n2)cc1]

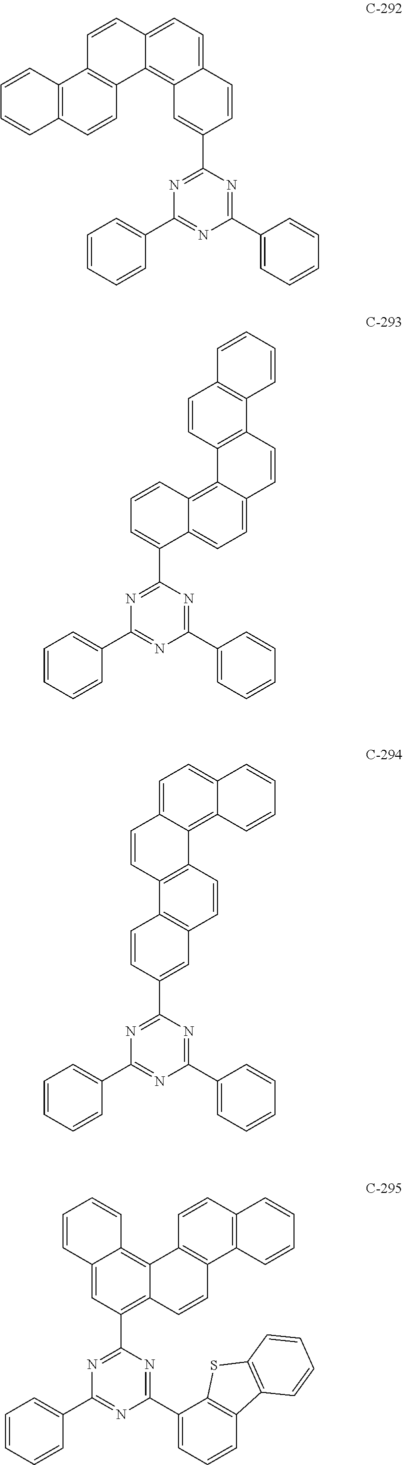 [SMILES: c1ccc(-c2nc(-c3ccccc3)nc(-c3ccc4ccc5ccc6c7ccccc7ccc6c5c4c3)n2)cc1 ; c1ccc(-c2nc(-c3ccccc3)nc(-c3cccc4c3ccc3ccc5c6ccccc6ccc5c34)n2)cc1 ; c1ccc(-c2nc(-c3ccccc3)nc(-c3ccc4c(ccc5c4ccc4ccc6ccccc6c45)c3)n2)cc1 ; c1ccc(-c2nc(-c3cc4ccccc4c4c3ccc3c5ccccc5ccc34)nc(-c3cccc4c3sc3ccccc34)n2)cc1]

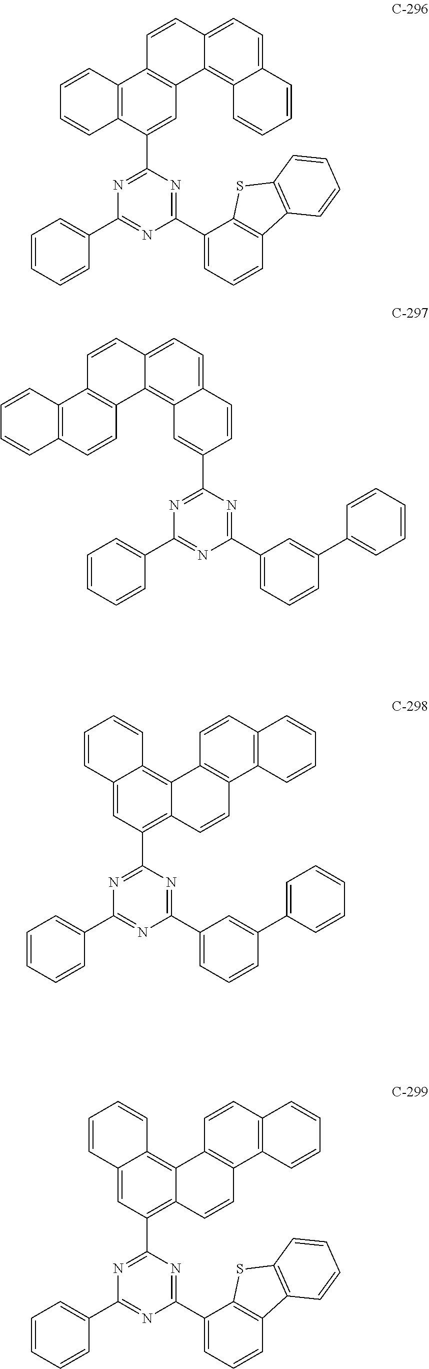 [SMILES: c1ccc(-c2nc(-c3cc4c(ccc5ccc6ccccc6c54)c4ccccc34)nc(-c3cccc4c3sc3ccccc34)n2)cc1 ; c1ccc(-c2cccc(-c3nc(-c4ccccc4)nc(-c4ccc5ccc6ccc7c8ccccc8ccc7c6c5c4)n3)c2)cc1 ; c1ccc(-c2cccc(-c3nc(-c4ccccc4)nc(-c4cc5ccccc5c5c4ccc4c6ccccc6ccc45)n3)c2)cc1 ; c1ccc(-c2nc(-c3cc4ccccc4c4c3ccc3c5ccccc5ccc34)nc(-c3cccc4c3sc3ccccc34)n2)cc1]

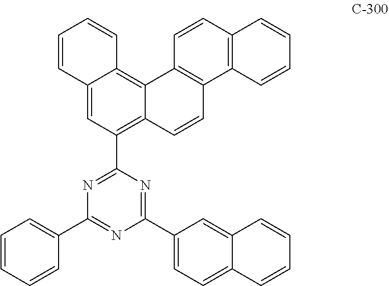 [SMILES: c1ccc(-c2nc(-c3ccc4ccccc4c3)nc(-c3cc4ccccc4c4c3ccc3c5ccccc5ccc34)n2)cc1]

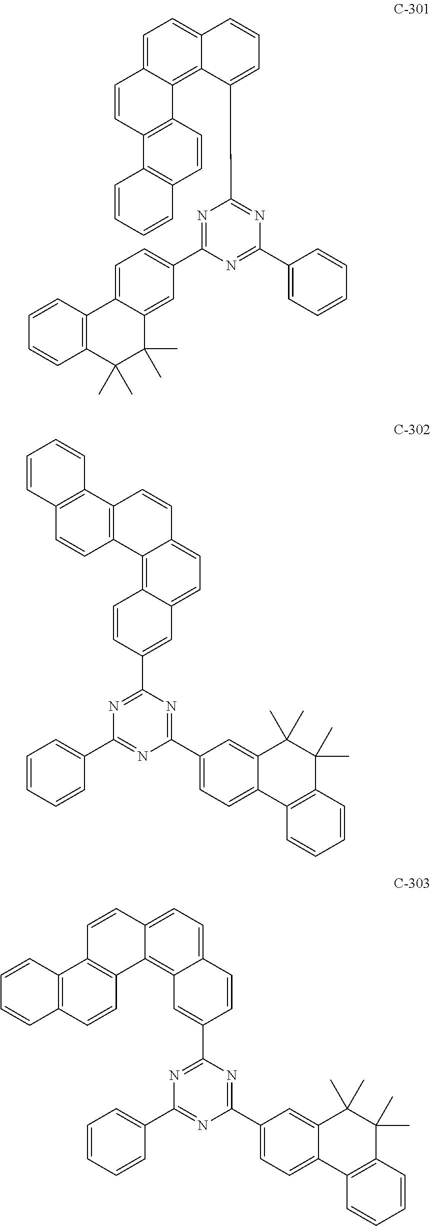 [SMILES: CC1(C)c2ccccc2-c2ccc(-c3nc(-c4ccccc4)nc(-c4cccc5ccc6ccc7c8ccccc8ccc7c6c45)n3)cc2C1(C)C ; CC1(C)c2ccccc2-c2ccc(-c3nc(-c4ccccc4)nc(-c4ccc5c(ccc6ccc7c8ccccc8ccc7c65)c4)n3)cc2C1(C)C ; CC1(C)c2ccccc2-c2ccc(-c3nc(-c4ccccc4)nc(-c4ccc5ccc6ccc7c8ccccc8ccc7c6c5c4)n3)cc2C1(C)C]

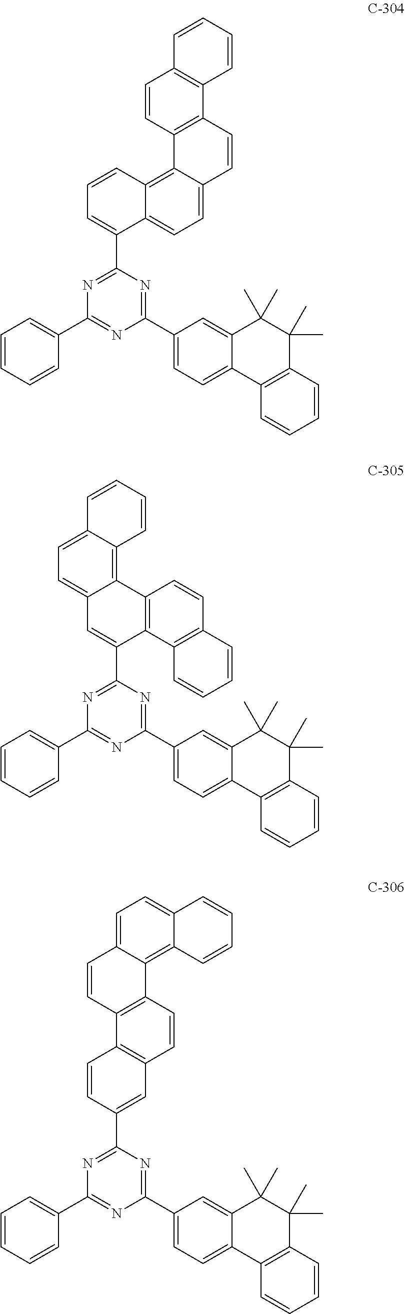 [SMILES: CC1(C)c2ccccc2-c2ccc(-c3nc(-c4ccccc4)nc(-c4cccc5c4ccc4ccc6c7ccccc7ccc6c45)n3)cc2C1(C)C ; CC1(C)c2ccccc2-c2ccc(-c3nc(-c4ccccc4)nc(-c4cc5ccc6ccccc6c5c5ccc6ccccc6c45)n3)cc2C1(C)C ; CC1(C)c2ccccc2-c2ccc(-c3nc(-c4ccccc4)nc(-c4ccc5c(ccc6c5ccc5ccc7ccccc7c56)c4)n3)cc2C1(C)C]

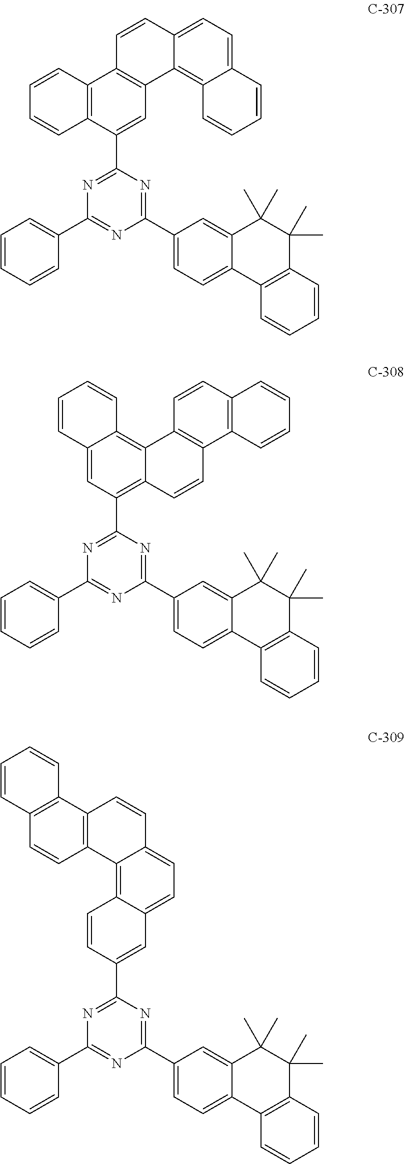 [SMILES: CC1(C)c2ccccc2-c2ccc(-c3nc(-c4ccccc4)nc(-c4cc5c(ccc6ccc7ccccc7c65)c5ccccc45)n3)cc2C1(C)C ; CC1(C)c2ccccc2-c2ccc(-c3nc(-c4ccccc4)nc(-c4cc5ccccc5c5c4ccc4c6ccccc6ccc45)n3)cc2C1(C)C ; CC1(C)c2ccccc2-c2ccc(-c3nc(-c4ccccc4)nc(-c4ccc5c(ccc6ccc7c8ccccc8ccc7c65)c4)n3)cc2C1(C)C]

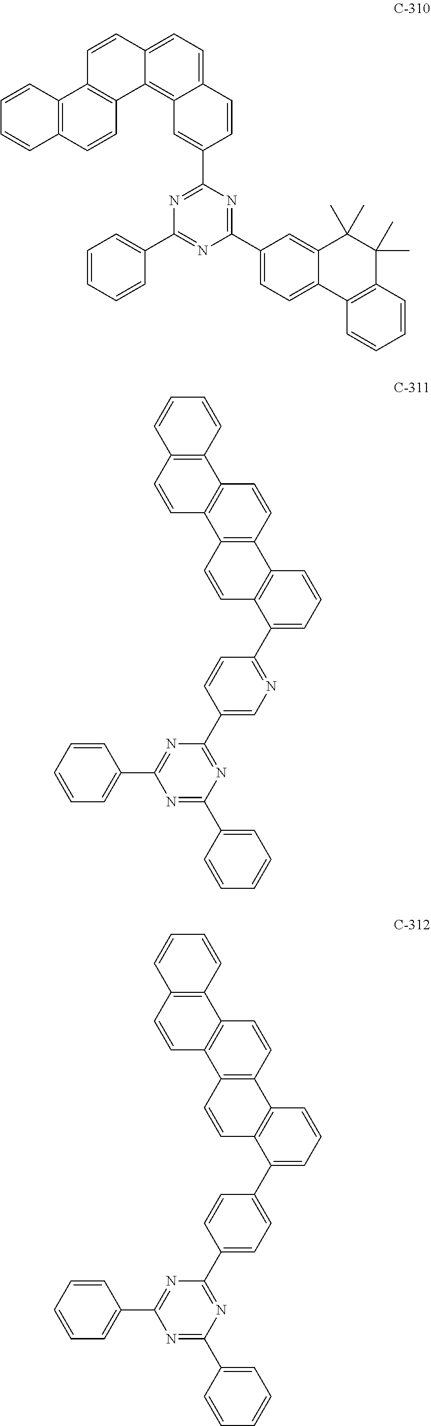 [SMILES: CC1(C)c2ccccc2-c2ccc(-c3nc(-c4ccccc4)nc(-c4ccc5ccc6ccc7c8ccccc8ccc7c6c5c4)n3)cc2C1(C)C ; c1ccc(-c2nc(-c3ccccc3)nc(-c3ccc(-c4cccc5c4ccc4c5ccc5c6ccccc6ccc54)nc3)n2)cc1 ; c1ccc(-c2nc(-c3ccccc3)nc(-c3ccc(-c4cccc5c4ccc4c5ccc5c6ccccc6ccc54)cc3)n2)cc1]

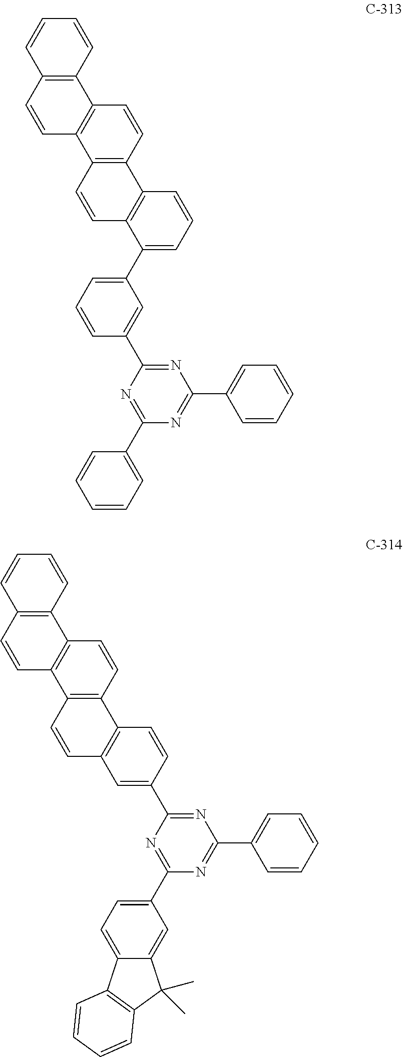 [SMILES: c1ccc(-c2nc(-c3ccccc3)nc(-c3cccc(-c4cccc5c4ccc4c5ccc5c6ccccc6ccc54)c3)n2)cc1 ; CC1(C)c2ccccc2-c2ccc(-c3nc(-c4ccccc4)nc(-c4ccc5c(ccc6c5ccc5c7ccccc7ccc56)c4)n3)cc21]

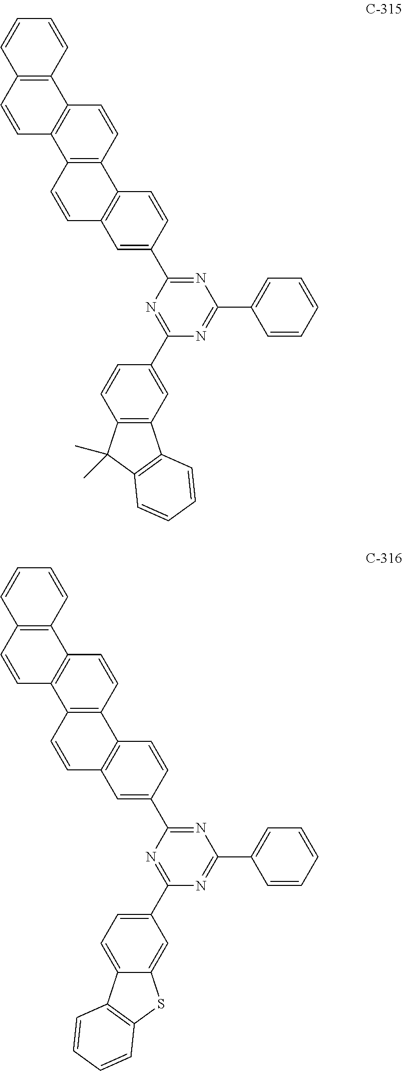 [SMILES: CC1(C)c2ccccc2-c2cc(-c3nc(-c4ccccc4)nc(-c4ccc5c(ccc6c5ccc5c7ccccc7ccc56)c4)n3)ccc21 ; c1ccc(-c2nc(-c3ccc4c(ccc5c4ccc4c6ccccc6ccc45)c3)nc(-c3ccc4c(c3)sc3ccccc34)n2)cc1]

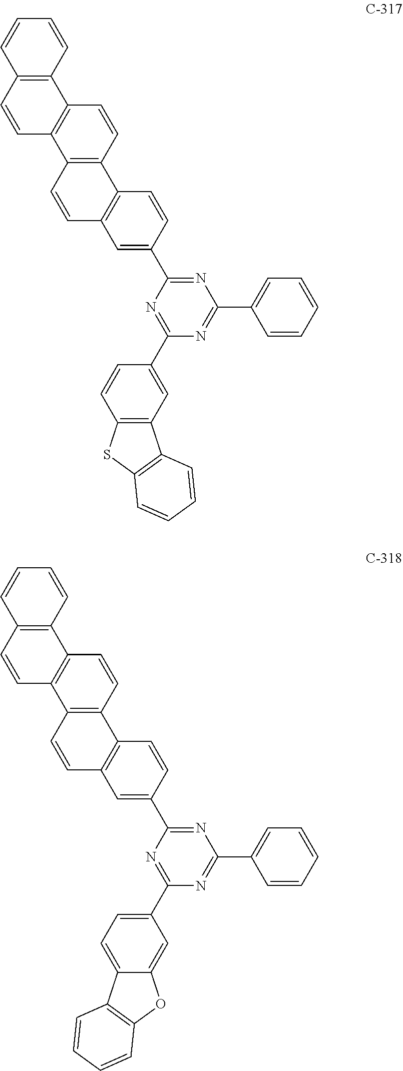 [SMILES: c1ccc(-c2nc(-c3ccc4c(ccc5c4ccc4c6ccccc6ccc45)c3)nc(-c3ccc4sc5ccccc5c4c3)n2)cc1 ; c1ccc(-c2nc(-c3ccc4c(ccc5c4ccc4c6ccccc6ccc45)c3)nc(-c3ccc4c(c3)oc3ccccc34)n2)cc1]

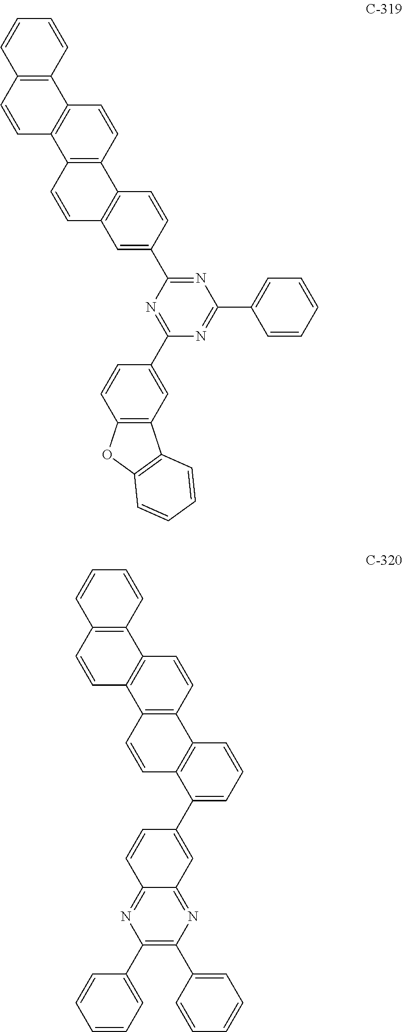 [SMILES: c1ccc(-c2nc(-c3ccc4c(ccc5c4ccc4c6ccccc6ccc45)c3)nc(-c3ccc4oc5ccccc5c4c3)n2)cc1 ; c1ccc(-c2nc3ccc(-c4cccc5c4ccc4c5ccc5c6ccccc6ccc54)cc3nc2-c2ccccc2)cc1]

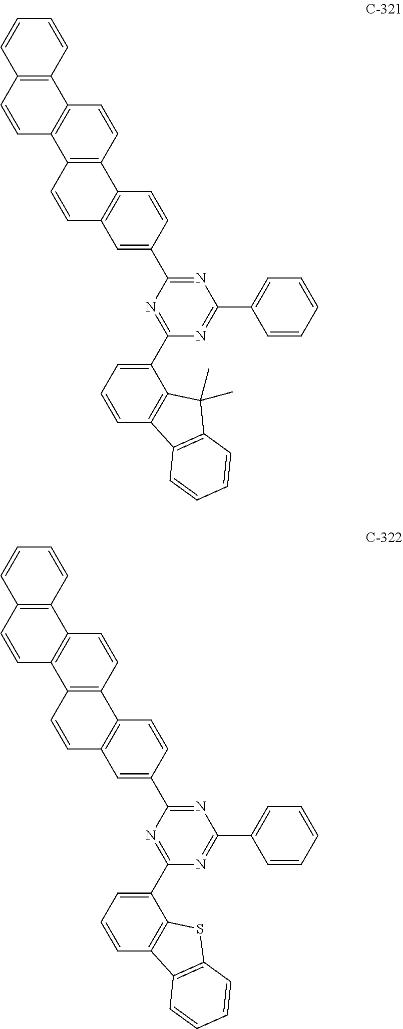 [SMILES: CC1(C)c2ccccc2-c2cccc(-c3nc(-c4ccccc4)nc(-c4ccc5c(ccc6c5ccc5c7ccccc7ccc56)c4)n3)c21 ; c1ccc(-c2nc(-c3ccc4c(ccc5c4ccc4c6ccccc6ccc45)c3)nc(-c3cccc4c3sc3ccccc34)n2)cc1]

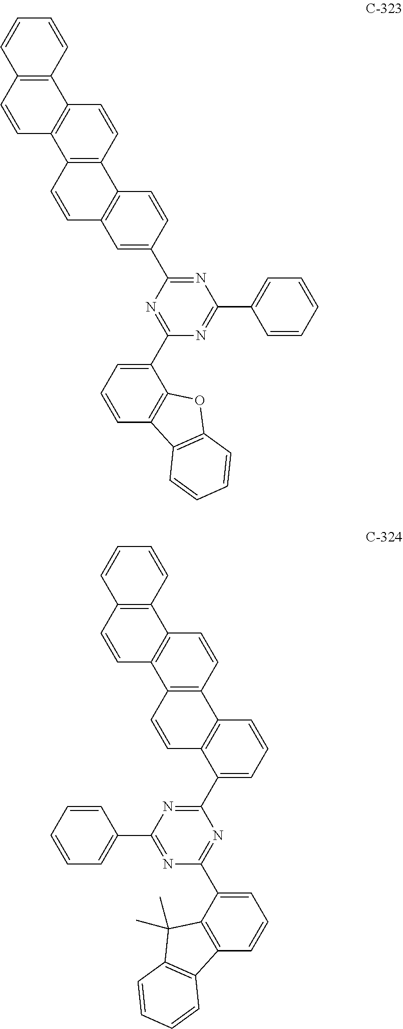 [SMILES: c1ccc(-c2nc(-c3ccc4c(ccc5c4ccc4c6ccccc6ccc45)c3)nc(-c3cccc4c3oc3ccccc34)n2)cc1 ; CC1(C)c2ccccc2-c2cccc(-c3nc(-c4ccccc4)nc(-c4cccc5c4ccc4c5ccc5c6ccccc6ccc54)n3)c21]

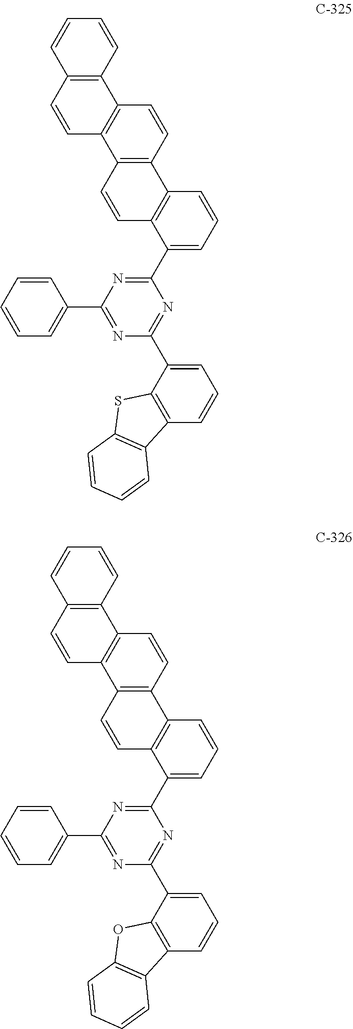 [SMILES: c1ccc(-c2nc(-c3cccc4c3ccc3c4ccc4c5ccccc5ccc43)nc(-c3cccc4c3sc3ccccc34)n2)cc1 ; c1ccc(-c2nc(-c3cccc4c3ccc3c4ccc4c5ccccc5ccc43)nc(-c3cccc4c3oc3ccccc34)n2)cc1]

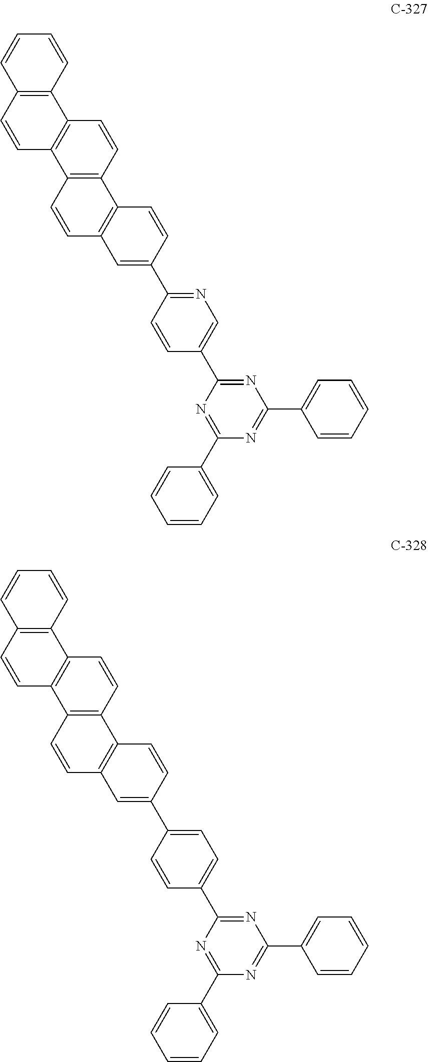 [SMILES: c1ccc(-c2nc(-c3ccccc3)nc(-c3ccc(-c4ccc5c(ccc6c5ccc5c7ccccc7ccc56)c4)nc3)n2)cc1 ; c1ccc(-c2nc(-c3ccccc3)nc(-c3ccc(-c4ccc5c(ccc6c5ccc5c7ccccc7ccc56)c4)cc3)n2)cc1]

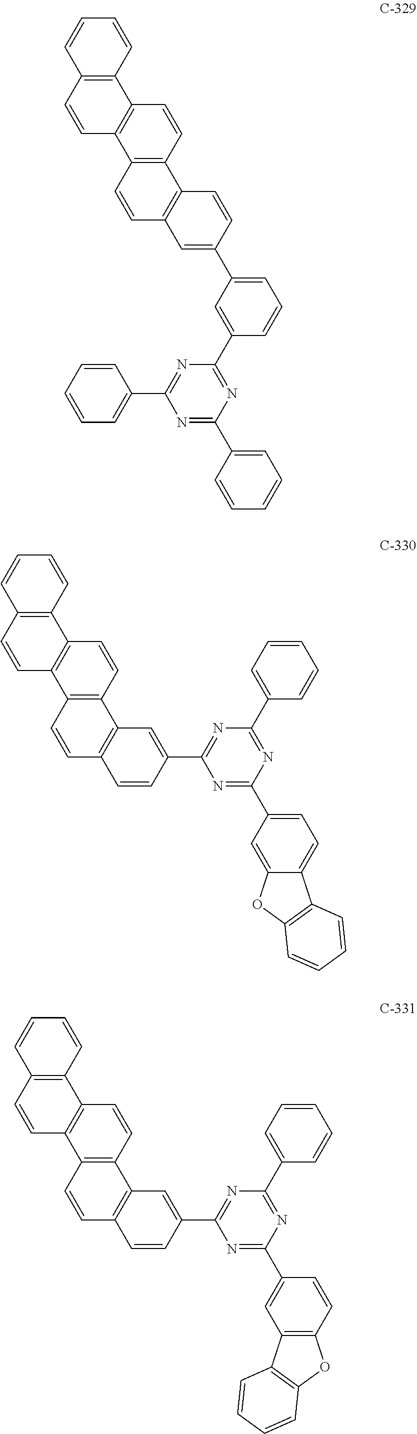 [SMILES: c1ccc(-c2nc(-c3ccccc3)nc(-c3cccc(-c4ccc5c(ccc6c5ccc5c7ccccc7ccc56)c4)c3)n2)cc1 ; c1ccc(-c2nc(-c3ccc4c(c3)oc3ccccc34)nc(-c3ccc4ccc5c(ccc6c7ccccc7ccc65)c4c3)n2)cc1 ; c1ccc(-c2nc(-c3ccc4ccc5c(ccc6c7ccccc7ccc65)c4c3)nc(-c3ccc4oc5ccccc5c4c3)n2)cc1]

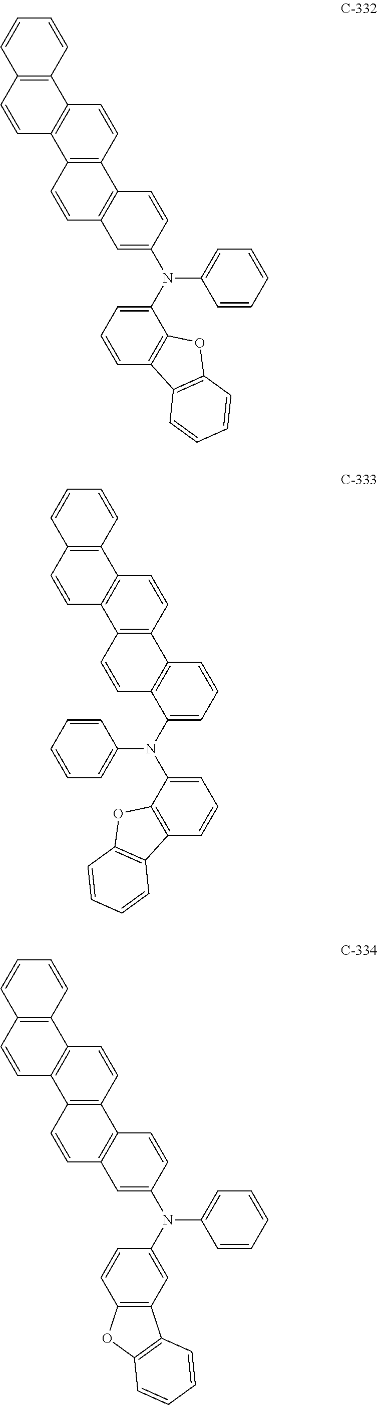 [SMILES: c1ccc(N(c2ccc3c(ccc4c3ccc3c5ccccc5ccc34)c2)c2cccc3c2oc2ccccc23)cc1 ; c1ccc(N(c2cccc3c2ccc2c3ccc3c4ccccc4ccc32)c2cccc3c2oc2ccccc23)cc1 ; c1ccc(N(c2ccc3c(ccc4c3ccc3c5ccccc5ccc34)c2)c2ccc3oc4ccccc4c3c2)cc1]

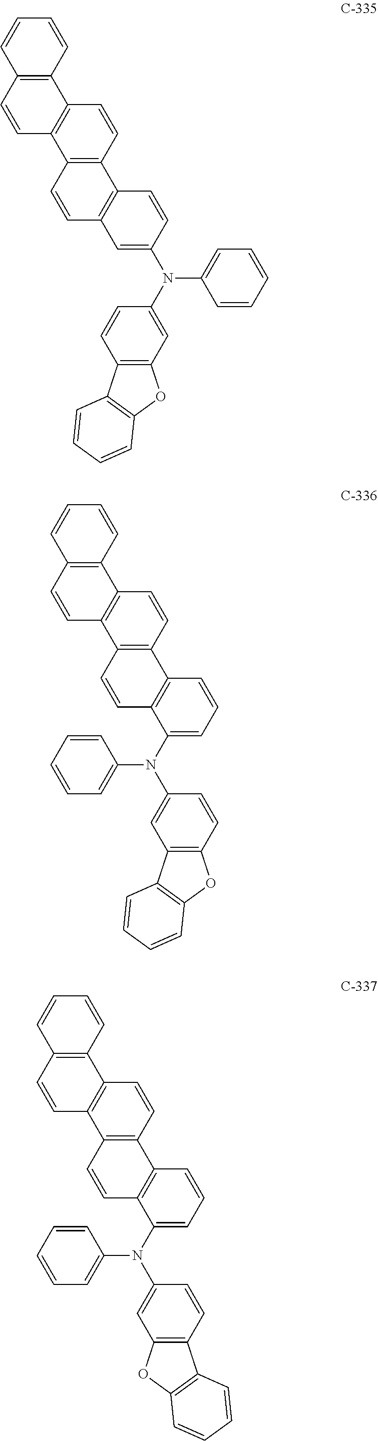

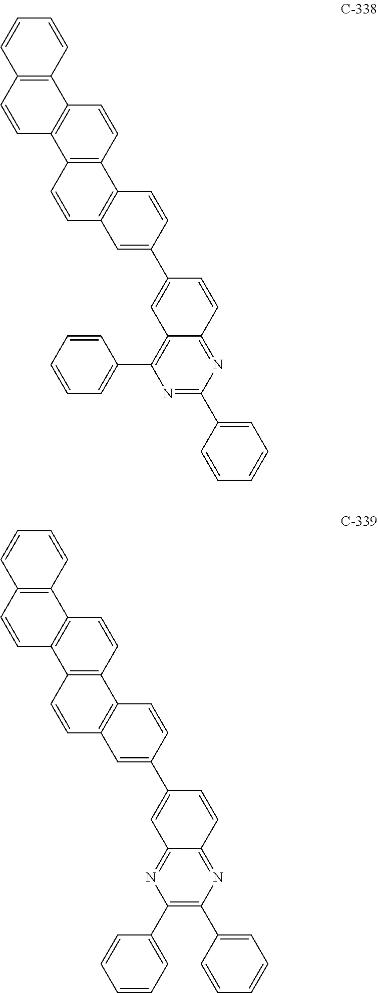 [SMILES: c1ccc(-c2nc(-c3ccccc3)c3cc(-c4ccc5c(ccc6c5ccc5c7ccccc7ccc56)c4)ccc3n2)cc1 ; c1ccc(-c2nc3ccc(-c4ccc5c(ccc6c5ccc5c7ccccc7ccc56)c4)cc3nc2-c2ccccc2)cc1]

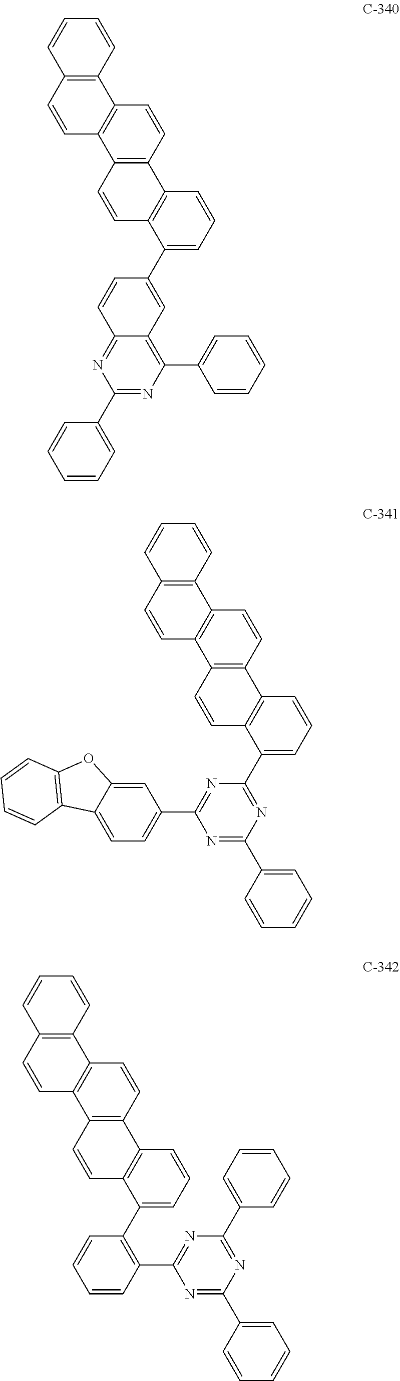 [SMILES: c1ccc(-c2nc(-c3ccccc3)c3cc(-c4cccc5c4ccc4c5ccc5c6ccccc6ccc54)ccc3n2)cc1 ; c1ccc(-c2nc(-c3ccc4c(c3)oc3ccccc34)nc(-c3cccc4c3ccc3c4ccc4c5ccccc5ccc43)n2)cc1 ; c1ccc(-c2nc(-c3ccccc3)nc(-c3ccccc3-c3cccc4c3ccc3c4ccc4c5ccccc5ccc43)n2)cc1]

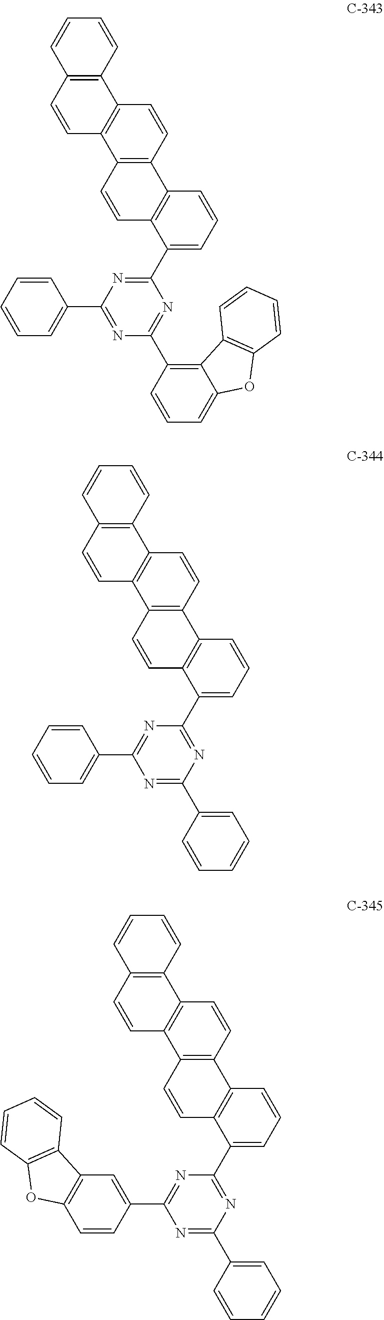 [SMILES: c1ccc(-c2nc(-c3cccc4c3ccc3c4ccc4c5ccccc5ccc43)nc(-c3cccc4oc5ccccc5c34)n2)cc1 ; c1ccc(-c2nc(-c3ccccc3)nc(-c3cccc4c3ccc3c4ccc4c5ccccc5ccc43)n2)cc1 ; c1ccc(-c2nc(-c3ccc4oc5ccccc5c4c3)nc(-c3cccc4c3ccc3c4ccc4c5ccccc5ccc43)n2)cc1]

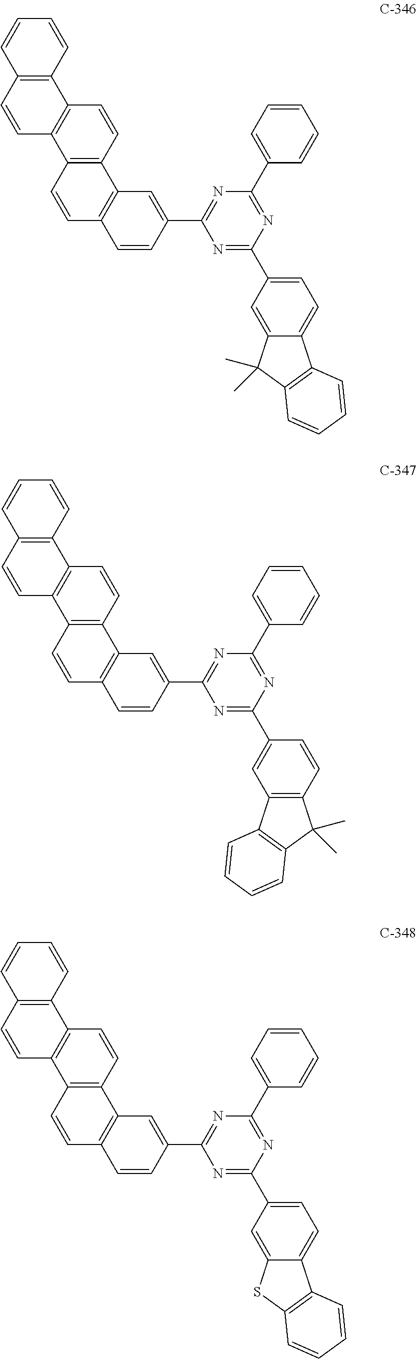 [SMILES: CC1(C)c2ccccc2-c2ccc(-c3nc(-c4ccccc4)nc(-c4ccc5ccc6c(ccc7c8ccccc8ccc76)c5c4)n3)cc21 ; CC1(C)c2ccccc2-c2cc(-c3nc(-c4ccccc4)nc(-c4ccc5ccc6c(ccc7c8ccccc8ccc76)c5c4)n3)ccc21 ; c1ccc(-c2nc(-c3ccc4c(c3)sc3ccccc34)nc(-c3ccc4ccc5c(ccc6c7ccccc7ccc65)c4c3)n2)cc1]

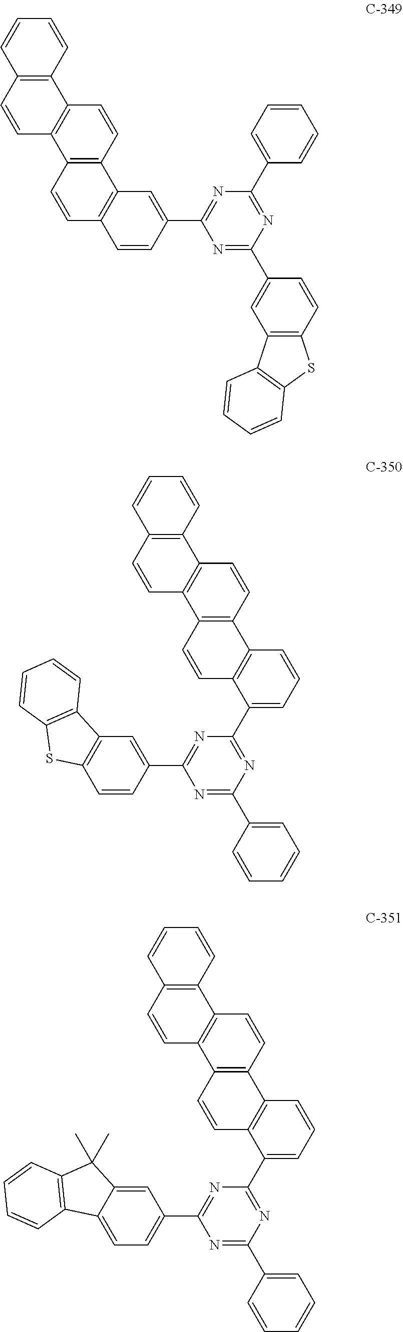 [SMILES: c1ccc(-c2nc(-c3ccc4ccc5c(ccc6c7ccccc7ccc65)c4c3)nc(-c3ccc4sc5ccccc5c4c3)n2)cc1 ; c1ccc(-c2nc(-c3ccc4sc5ccccc5c4c3)nc(-c3cccc4c3ccc3c4ccc4c5ccccc5ccc43)n2)cc1 ; CC1(C)c2ccccc2-c2ccc(-c3nc(-c4ccccc4)nc(-c4cccc5c4ccc4c5ccc5c6ccccc6ccc54)n3)cc21]

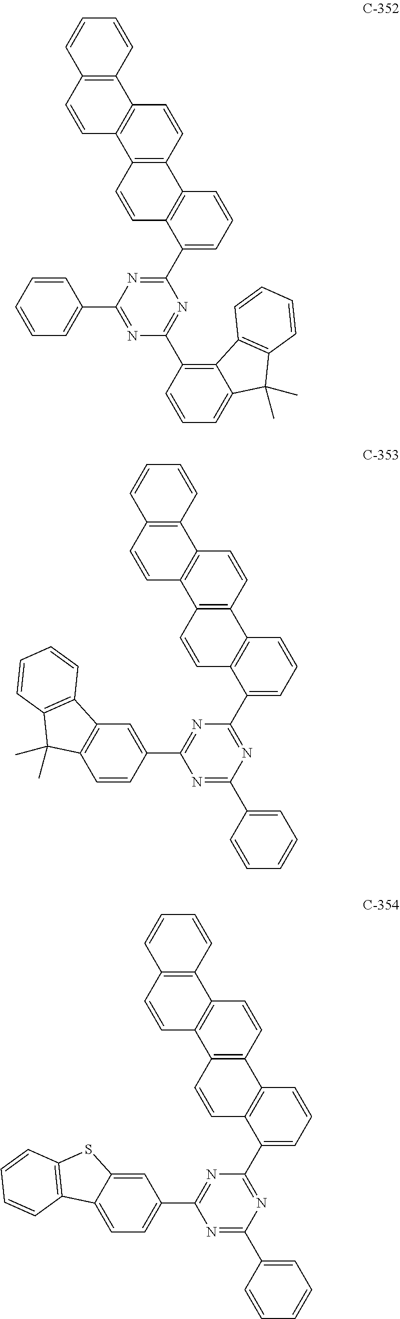

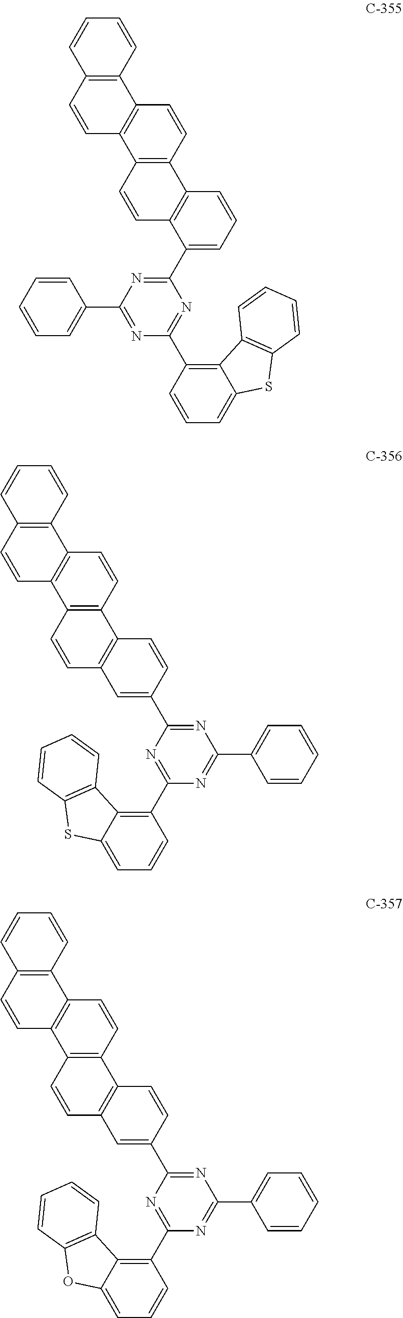 [SMILES: c1ccc(-c2nc(-c3cccc4c3ccc3c4ccc4c5ccccc5ccc43)nc(-c3cccc4sc5ccccc5c34)n2)cc1 ; c1ccc(-c2nc(-c3ccc4c(ccc5c4ccc4c6ccccc6ccc45)c3)nc(-c3cccc4sc5ccccc5c34)n2)cc1 ; c1ccc(-c2nc(-c3ccc4c(ccc5c4ccc4c6ccccc6ccc45)c3)nc(-c3cccc4oc5ccccc5c34)n2)cc1]

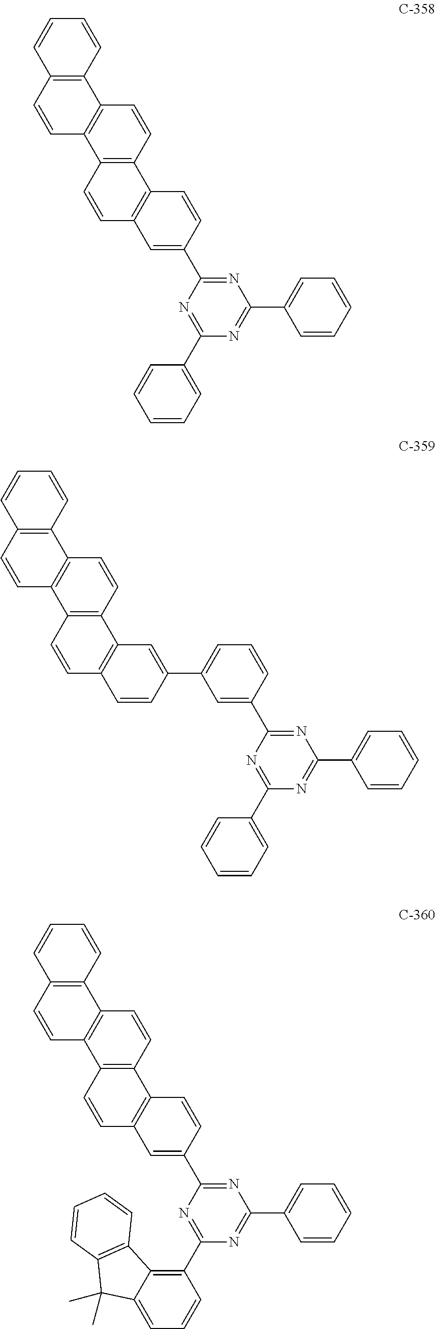 [SMILES: c1ccc(-c2nc(-c3ccccc3)nc(-c3ccc4c(ccc5c4ccc4c6ccccc6ccc45)c3)n2)cc1 ; c1ccc(-c2nc(-c3ccccc3)nc(-c3cccc(-c4ccc5ccc6c(ccc7c8ccccc8ccc76)c5c4)c3)n2)cc1 ; CC1(C)c2ccccc2-c2c(-c3nc(-c4ccccc4)nc(-c4ccc5c(ccc6c5ccc5c7ccccc7ccc56)c4)n3)cccc21]

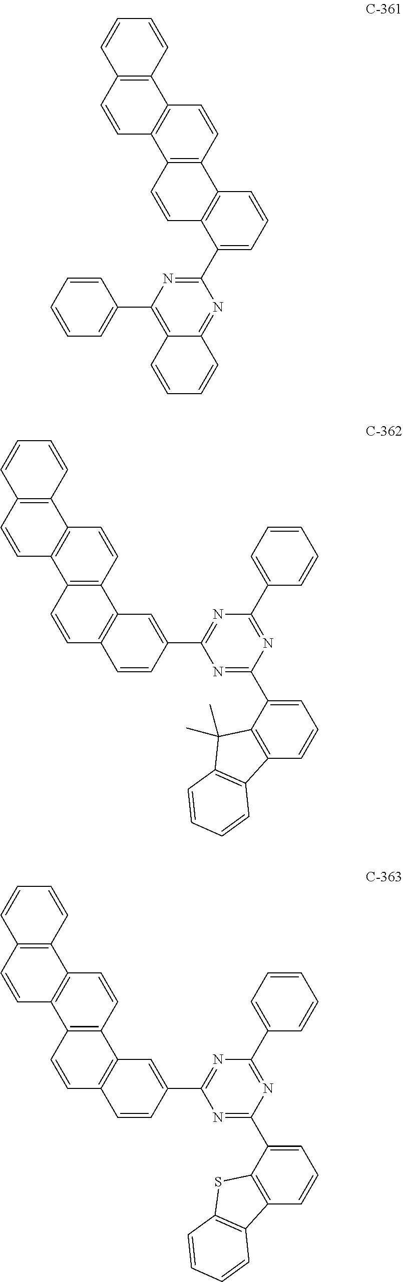 [SMILES: c1ccc(-c2nc(-c3cccc4c3ccc3c4ccc4c5ccccc5ccc43)nc3ccccc23)cc1 ; CC1(C)c2ccccc2-c2cccc(-c3nc(-c4ccccc4)nc(-c4ccc5ccc6c(ccc7c8ccccc8ccc76)c5c4)n3)c21 ; c1ccc(-c2nc(-c3ccc4ccc5c(ccc6c7ccccc7ccc65)c4c3)nc(-c3cccc4c3sc3ccccc34)n2)cc1]

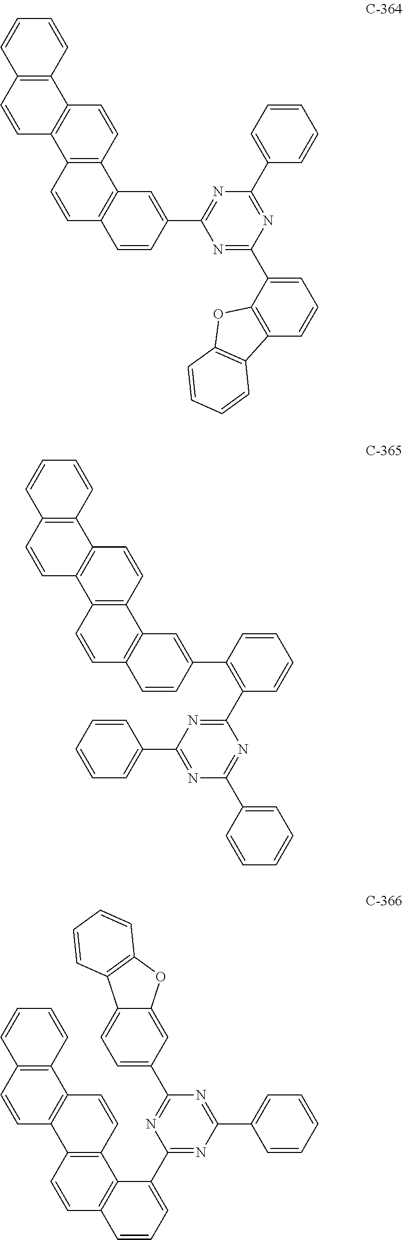 [SMILES: c1ccc(-c2nc(-c3ccc4ccc5c(ccc6c7ccccc7ccc65)c4c3)nc(-c3cccc4c3oc3ccccc34)n2)cc1 ; c1ccc(-c2nc(-c3ccccc3)nc(-c3ccccc3-c3ccc4ccc5c(ccc6c7ccccc7ccc65)c4c3)n2)cc1 ; c1ccc(-c2nc(-c3ccc4c(c3)oc3ccccc34)nc(-c3cccc4ccc5c6ccc7ccccc7c6ccc5c34)n2)cc1]

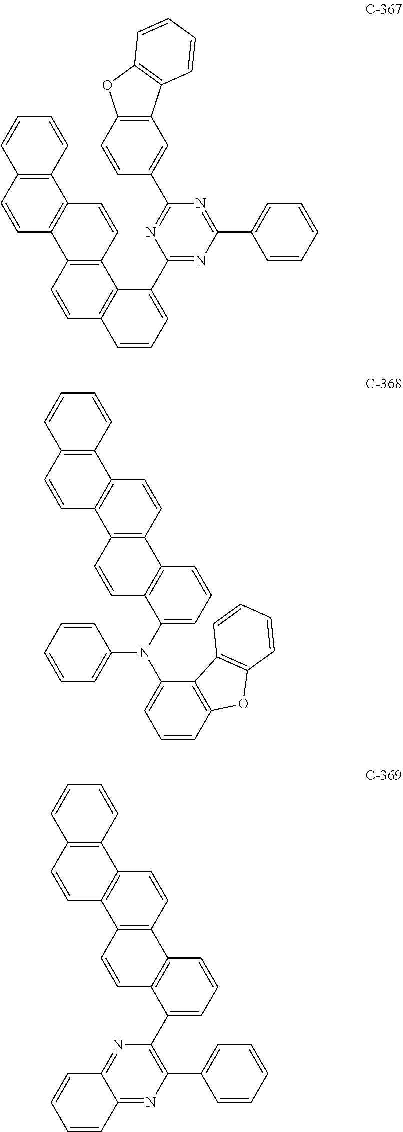 [SMILES: c1ccc(-c2nc(-c3ccc4oc5ccccc5c4c3)nc(-c3cccc4ccc5c6ccc7ccccc7c6ccc5c34)n2)cc1 ; c1ccc(N(c2cccc3c2ccc2c3ccc3c4ccccc4ccc32)c2cccc3oc4ccccc4c23)cc1 ; c1ccc(-c2nc3ccccc3nc2-c2cccc3c2ccc2c3ccc3c4ccccc4ccc32)cc1]

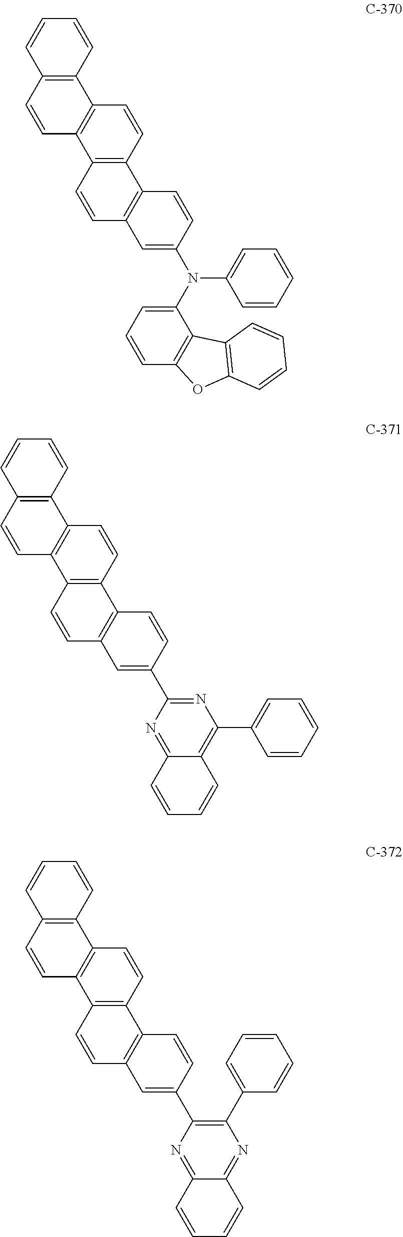 [SMILES: c1ccc(N(c2ccc3c(ccc4c3ccc3c5ccccc5ccc34)c2)c2cccc3oc4ccccc4c23)cc1 ; c1ccc(-c2nc(-c3ccc4c(ccc5c4ccc4c6ccccc6ccc45)c3)nc3ccccc23)cc1 ; c1ccc(-c2nc3ccccc3nc2-c2ccc3c(ccc4c3ccc3c5ccccc5ccc34)c2)cc1]

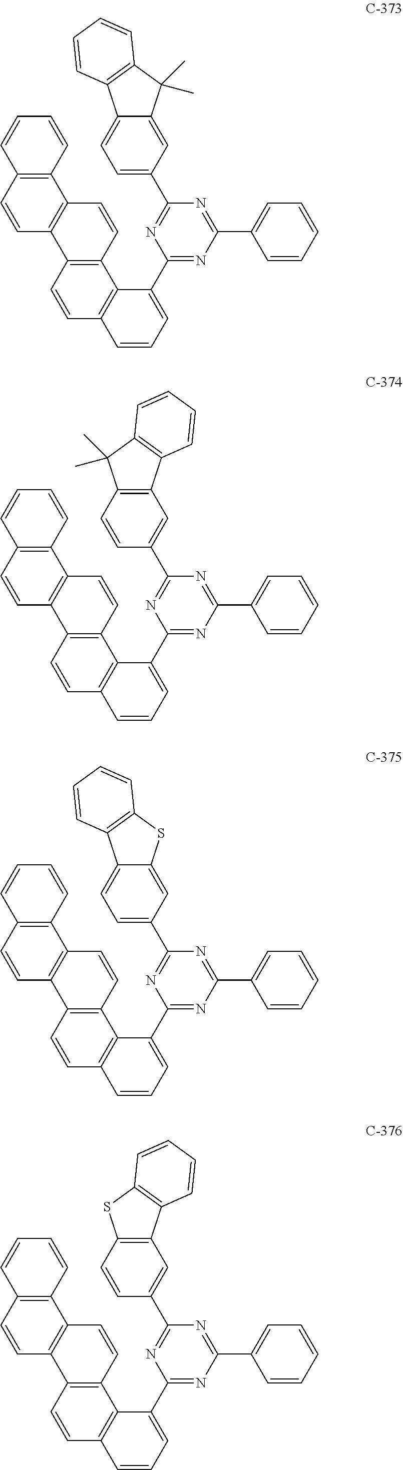 [SMILES: CC1(C)c2ccccc2-c2ccc(-c3nc(-c4ccccc4)nc(-c4cccc5ccc6c7ccc8ccccc8c7ccc6c45)n3)cc21 ; CC1(C)c2ccccc2-c2cc(-c3nc(-c4ccccc4)nc(-c4cccc5ccc6c7ccc8ccccc8c7ccc6c45)n3)ccc21 ; c1ccc(-c2nc(-c3ccc4c(c3)sc3ccccc34)nc(-c3cccc4ccc5c6ccc7ccccc7c6ccc5c34)n2)cc1 ; c1ccc(-c2nc(-c3ccc4sc5ccccc5c4c3)nc(-c3cccc4ccc5c6ccc7ccccc7c6ccc5c34)n2)cc1]

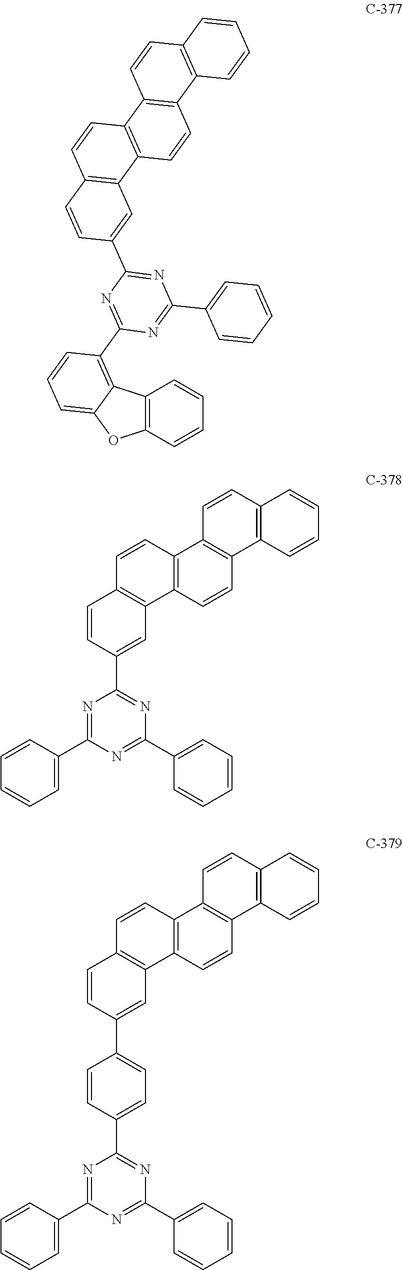 [SMILES: c1ccc(-c2nc(-c3ccc4ccc5c(ccc6c7ccccc7ccc65)c4c3)nc(-c3cccc4oc5ccccc5c34)n2)cc1 ; c1ccc(-c2nc(-c3ccccc3)nc(-c3ccc4ccc5c(ccc6c7ccccc7ccc65)c4c3)n2)cc1 ; c1ccc(-c2nc(-c3ccccc3)nc(-c3ccc(-c4ccc5ccc6c(ccc7c8ccccc8ccc76)c5c4)cc3)n2)cc1]

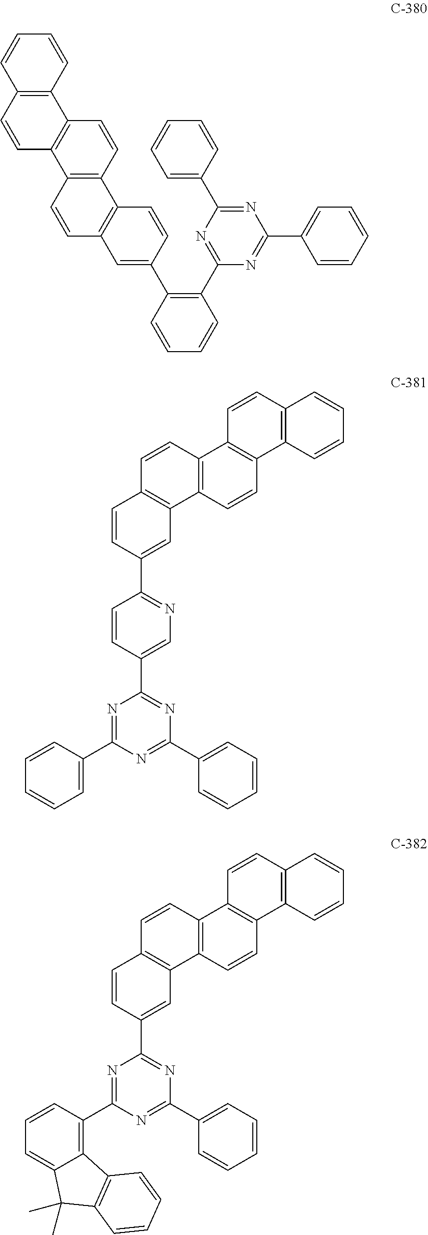 [SMILES: c1ccc(-c2nc(-c3ccccc3)nc(-c3ccccc3-c3ccc4c(ccc5c4ccc4c6ccccc6ccc45)c3)n2)cc1 ; c1ccc(-c2nc(-c3ccccc3)nc(-c3ccc(-c4ccc5ccc6c(ccc7c8ccccc8ccc76)c5c4)nc3)n2)cc1 ; CC1(C)c2ccccc2-c2c(-c3nc(-c4ccccc4)nc(-c4ccc5ccc6c(ccc7c8ccccc8ccc76)c5c4)n3)cccc21]

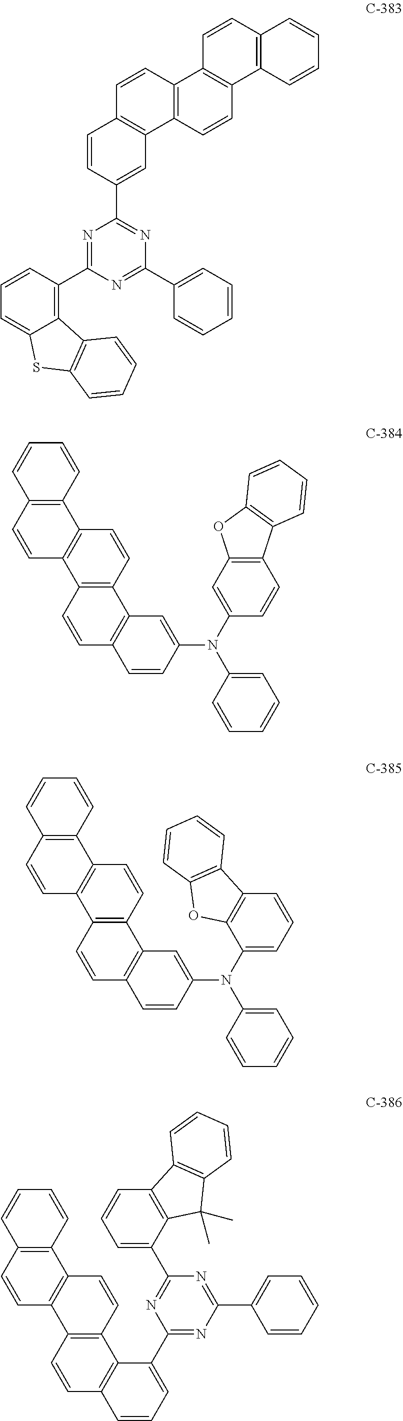 [SMILES: c1ccc(-c2nc(-c3ccc4ccc5c(ccc6c7ccccc7ccc65)c4c3)nc(-c3cccc4sc5ccccc5c34)n2)cc1 ; c1ccc(N(c2ccc3c(c2)oc2ccccc23)c2ccc3ccc4c(ccc5c6ccccc6ccc54)c3c2)cc1 ; c1ccc(N(c2ccc3ccc4c(ccc5c6ccccc6ccc54)c3c2)c2cccc3c2oc2ccccc23)cc1 ; CC1(C)c2ccccc2-c2cccc(-c3nc(-c4ccccc4)nc(-c4cccc5ccc6c7ccc8ccccc8c7ccc6c45)n3)c21]

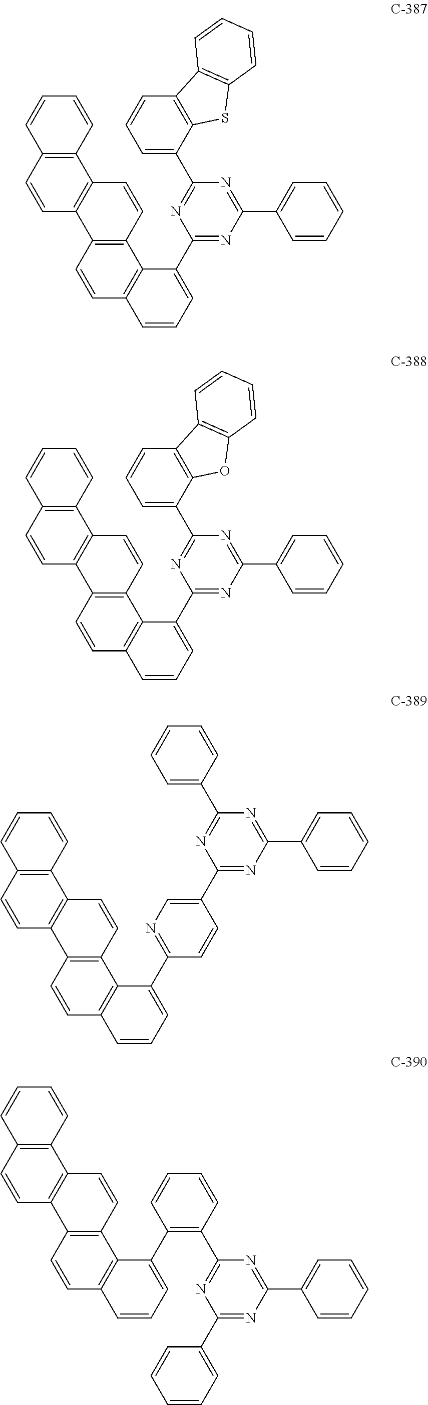 [SMILES: c1ccc(-c2nc(-c3cccc4c3sc3ccccc34)nc(-c3cccc4ccc5c6ccc7ccccc7c6ccc5c34)n2)cc1 ; c1ccc(-c2nc(-c3cccc4c3oc3ccccc34)nc(-c3cccc4ccc5c6ccc7ccccc7c6ccc5c34)n2)cc1 ; c1ccc(-c2nc(-c3ccccc3)nc(-c3ccc(-c4cccc5ccc6c7ccc8ccccc8c7ccc6c45)nc3)n2)cc1 ; c1ccc(-c2nc(-c3ccccc3)nc(-c3ccccc3-c3cccc4ccc5c6ccc7ccccc7c6ccc5c34)n2)cc1]

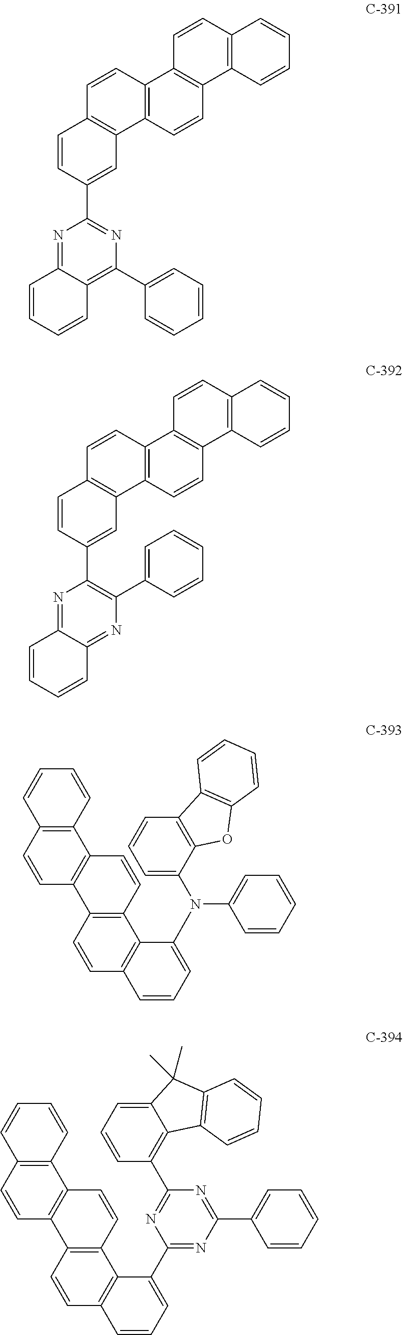 [SMILES: c1ccc(-c2nc(-c3ccc4ccc5c(ccc6c7ccccc7ccc65)c4c3)nc3ccccc23)cc1 ; c1ccc(-c2nc3ccccc3nc2-c2ccc3ccc4c(ccc5c6ccccc6ccc54)c3c2)cc1 ; c1ccc(N(c2cccc3c2oc2ccccc23)c2cccc3ccc4c5ccc6ccccc6c5ccc4c23)cc1 ; CC1(C)c2ccccc2-c2c(-c3nc(-c4ccccc4)nc(-c4cccc5ccc6c7ccc8ccccc8c7ccc6c45)n3)cccc21]

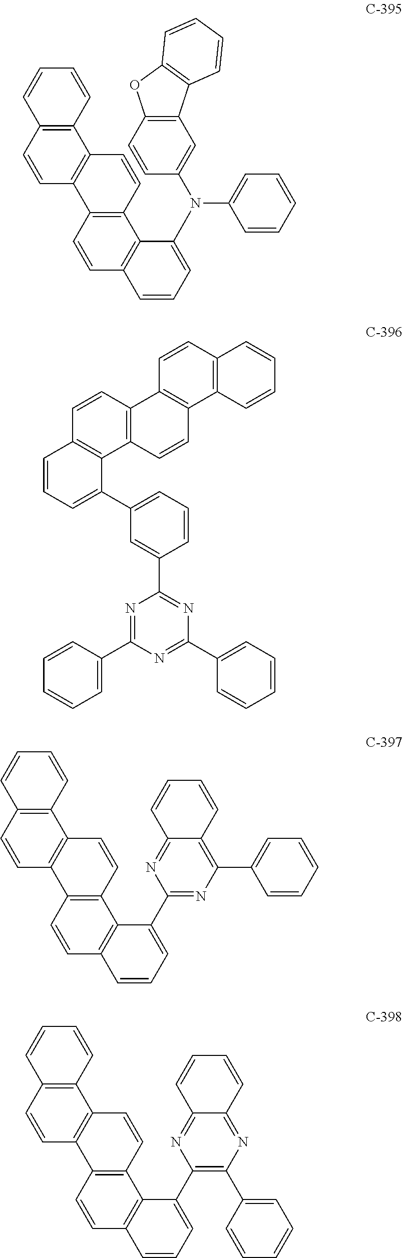

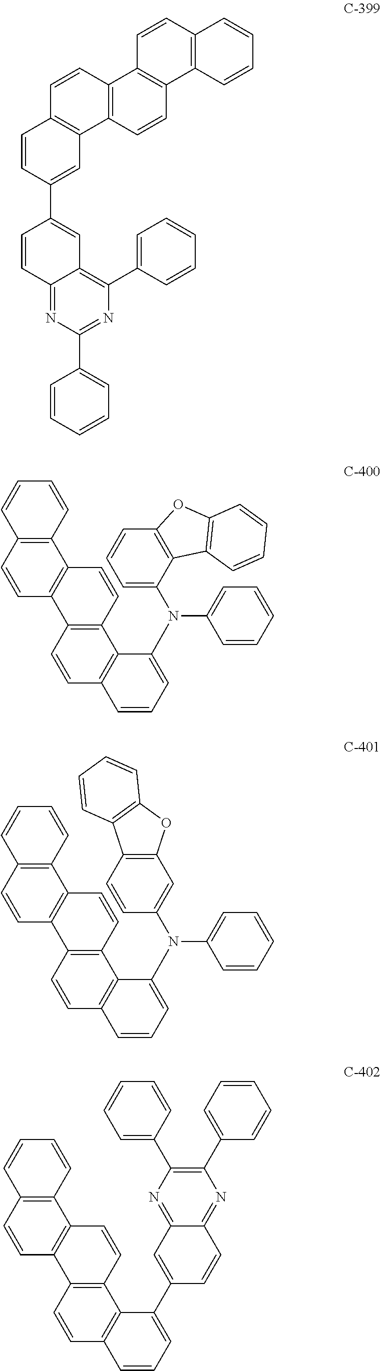

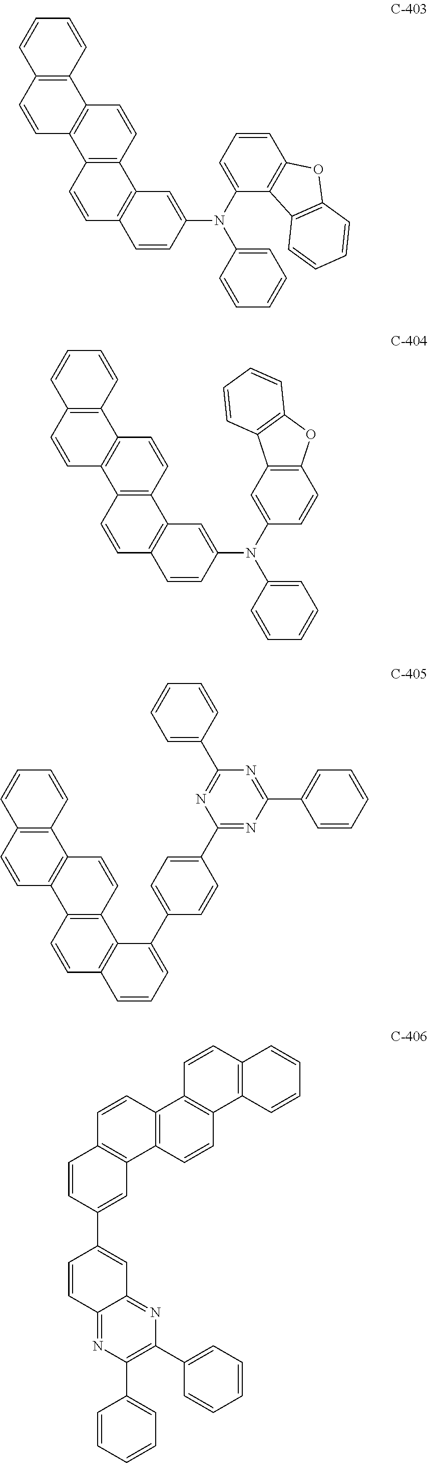 [SMILES: c1ccc(N(c2ccc3ccc4c(ccc5c6ccccc6ccc54)c3c2)c2cccc3oc4ccccc4c23)cc1 ; c1ccc(N(c2ccc3ccc4c(ccc5c6ccccc6ccc54)c3c2)c2ccc3oc4ccccc4c3c2)cc1 ; c1ccc(-c2nc(-c3ccccc3)nc(-c3ccc(-c4cccc5ccc6c7ccc8ccccc8c7ccc6c45)cc3)n2)cc1 ; c1ccc(-c2nc3ccc(-c4ccc5ccc6c(ccc7c8ccccc8ccc76)c5c4)cc3nc2-c2ccccc2)cc1]

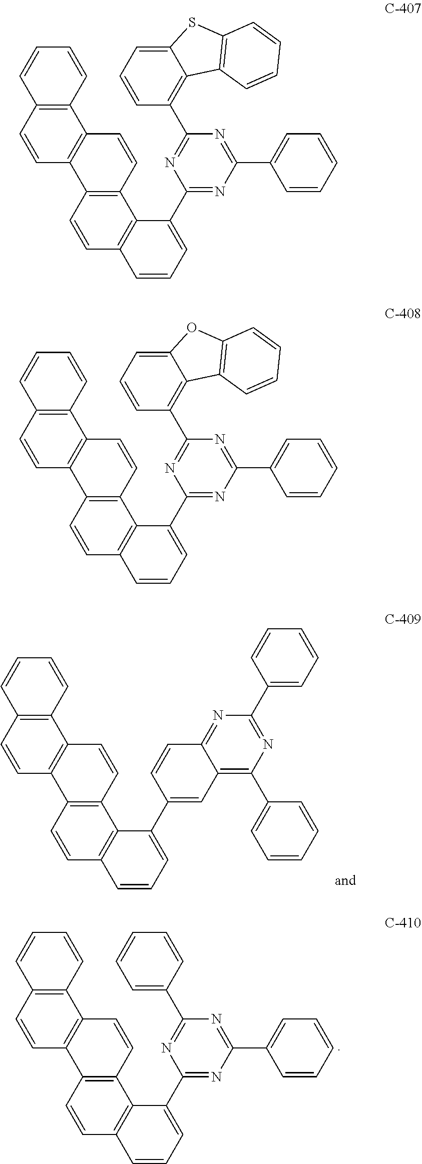

6. A plurality of host materials comprising a first host material including the organic electroluminescent compound according to claim 1, and a second host material which is different from the first host material.

7. The plurality of host materials according to claim 6, wherein the second host material comprises a compound represented by the following formula 2: 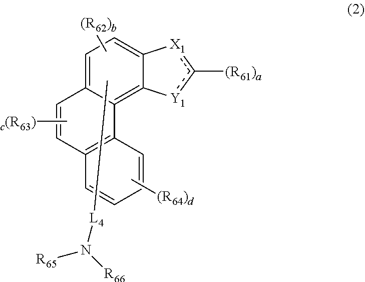
wherein
X₁ and Y₁ each independently represent —N═, —NR₆₇—, —O— or —S—, provided that one of X₁ and Y₁ is —N═, and the other of X₁ and Y₁ is —NR₆₇—, —O— or —S—;
R₆₁ represents a substituted or unsubstituted (C6-C30)aryl or a substituted or unsubstituted (3- to 30-membered)heteroaryl;
R₆₂ to R₆₄ and R₆₇ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of a (C3-C30) aliphatic ring and a (C6-C30) aromatic ring, or -L₃″-N(Ar₃″)(Ar₄″); or may be linked to the adjacent substituent each other to form a ring(s);
L₃″ represents a single bond, a substituted or unsubstituted (C6-C30)arylene, or a substituted or unsubstituted (3- to 30-membered)heteroarylene;
Ar₃″ and Ar₄″ each independently represent hydrogen, a substituted or unsubstituted (C1-C30)alkyl, a substituted or unsubstituted (C2-C30)alkenyl, a substituted or unsubstituted fused ring of a (C3-C30) aliphatic ring and a (C6-C30) aromatic ring, a substituted or unsubstituted (C6-C30)aryl, or a substituted or unsubstituted (3- to 30-membered)heteroaryl;
L₄ represents a single bond, a substituted or unsubstituted (C6-C30)arylene, or a substituted or unsubstituted (3- to 30-membered)heteroarylene;
R₆₅ and R₆₆ each independently represent a substituted or unsubstituted (C6-C30)aryl or a substituted or unsubstituted (3- to 30-membered)heteroaryl; and
a represents 1, b and c each independently represent 1 or 2, and d is an integer of 1 to 4, and when b to d are an integer of 2 or more, each of R₆₂ to R₆₄ may be the same or different.

8. The plurality of host materials according to claim 7, wherein the compound represented by the formula 2 is selected from the following compounds:

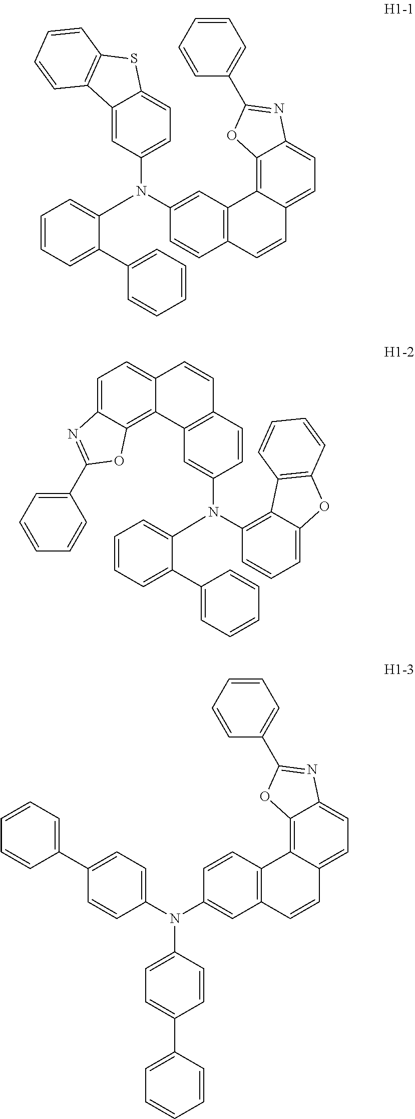

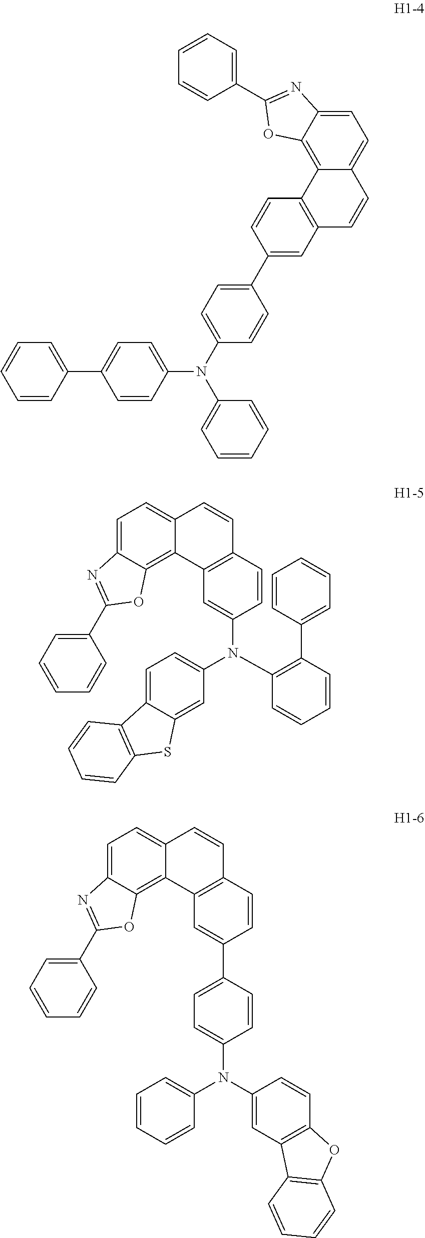 [SMILES: c1ccc(-c2ccc(N(c3ccccc3)c3ccc(-c4ccc5c(ccc6ccc7nc(-c8ccccc8)oc7c65)c4)cc3)cc2)cc1 ; c1ccc(-c2nc3ccc4ccc5ccc(N(c6ccc7c(c6)sc6ccccc67)c6ccccc6-c6ccccc6)cc5c4c3o2)cc1 ; c1ccc(-c2nc3ccc4ccc5ccc(-c6ccc(N(c7ccccc7)c7ccc8oc9ccccc9c8c7)cc6)cc5c4c3o2)cc1]

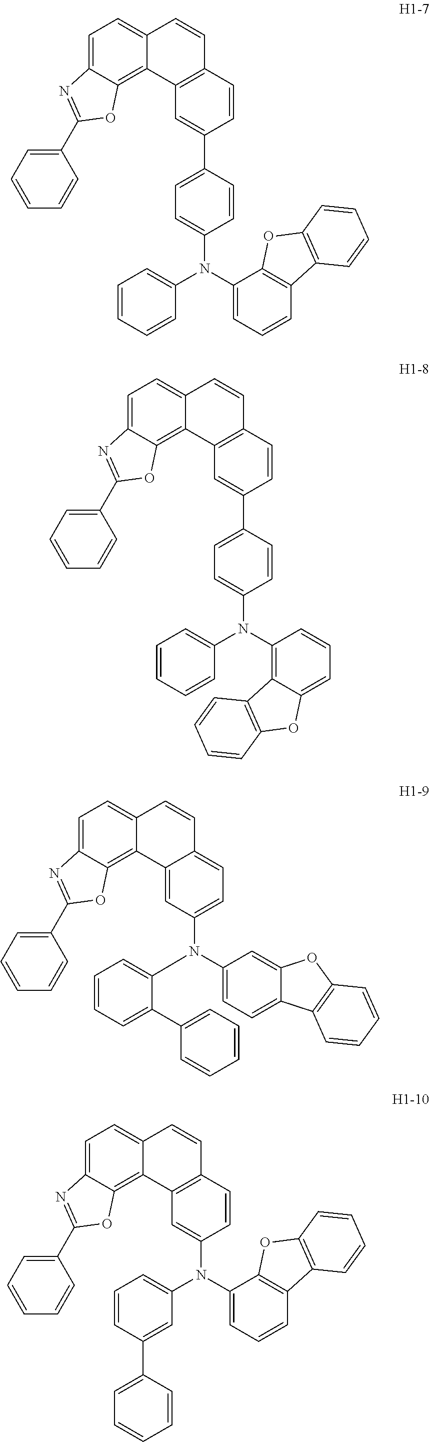

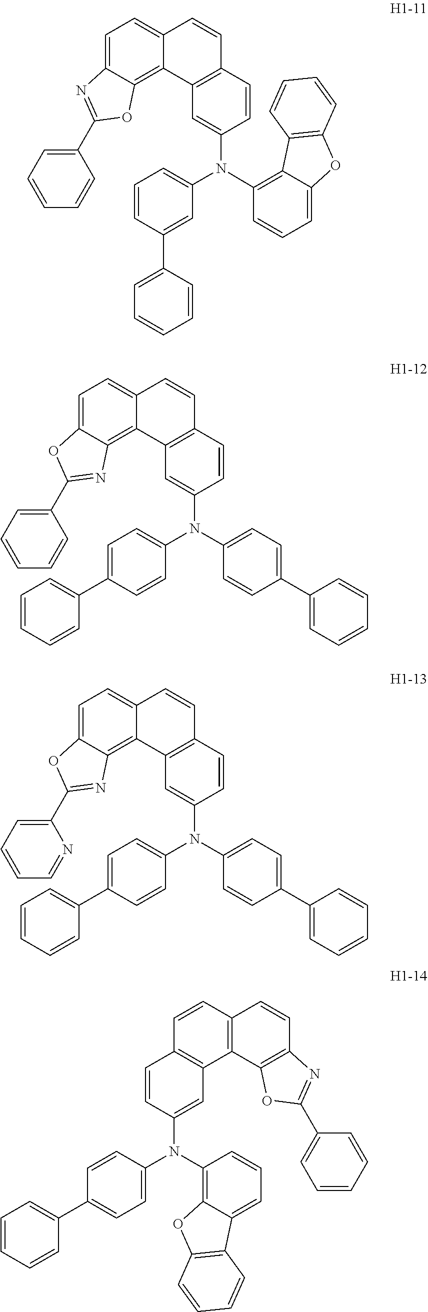

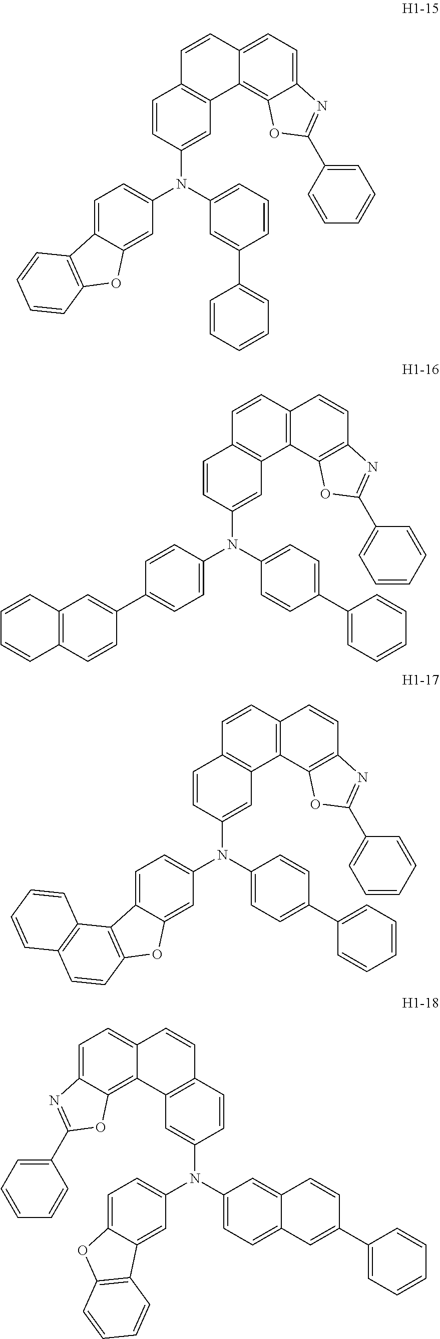

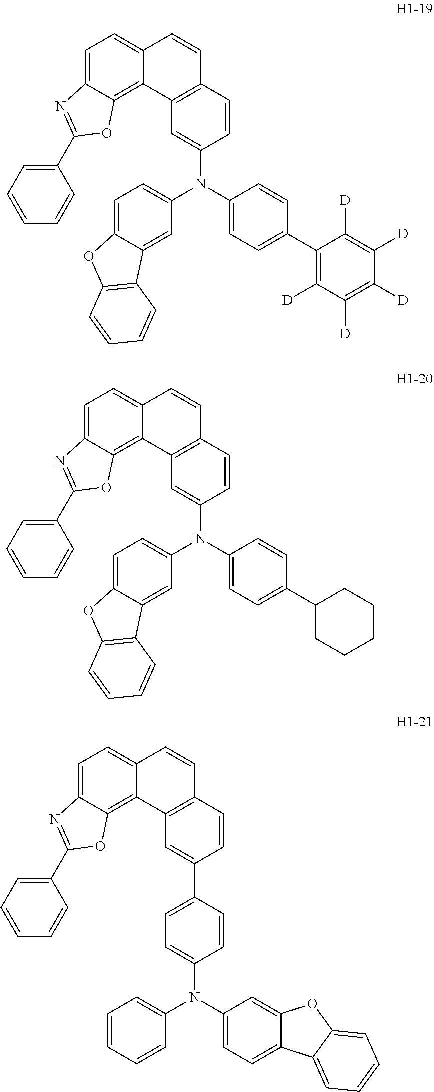

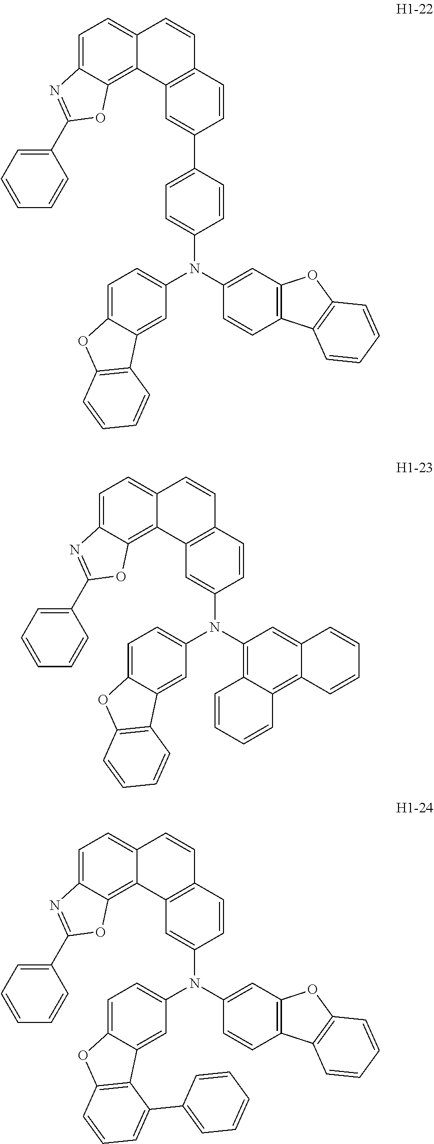 [SMILES: c1ccc(-c2nc3ccc4ccc5ccc(-c6ccc(N(c7ccc8c(c7)oc7ccccc78)c7ccc8oc9ccccc9c8c7)cc6)cc5c4c3o2)cc1 ; c1ccc(-c2nc3ccc4ccc5ccc(N(c6ccc7oc8ccccc8c7c6)c6cc7ccccc7c7ccccc67)cc5c4c3o2)cc1 ; c1ccc(-c2nc3ccc4ccc5ccc(N(c6ccc7c(c6)oc6ccccc67)c6ccc7oc8cccc(-c9ccccc9)c8c7c6)cc5c4c3o2)cc1]

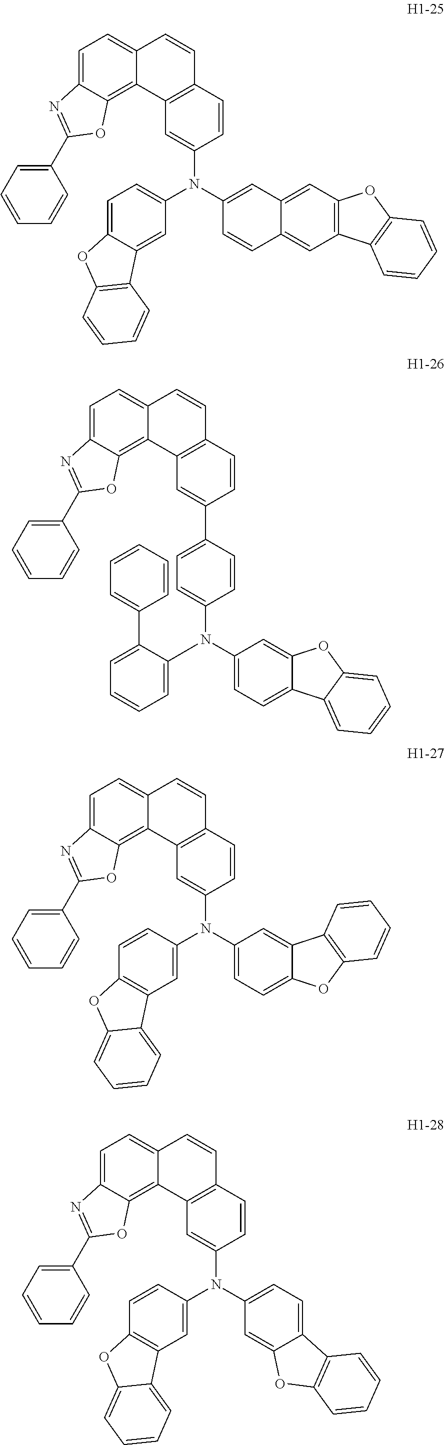 [SMILES: c1ccc(-c2nc3ccc4ccc5ccc(N(c6ccc7cc8c(cc7c6)oc6ccccc68)c6ccc7oc8ccccc8c7c6)cc5c4c3o2)cc1 ; c1ccc(-c2nc3ccc4ccc5ccc(-c6ccc(N(c7ccc8c(c7)oc7ccccc78)c7ccccc7-c7ccccc7)cc6)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3o2)cc1]

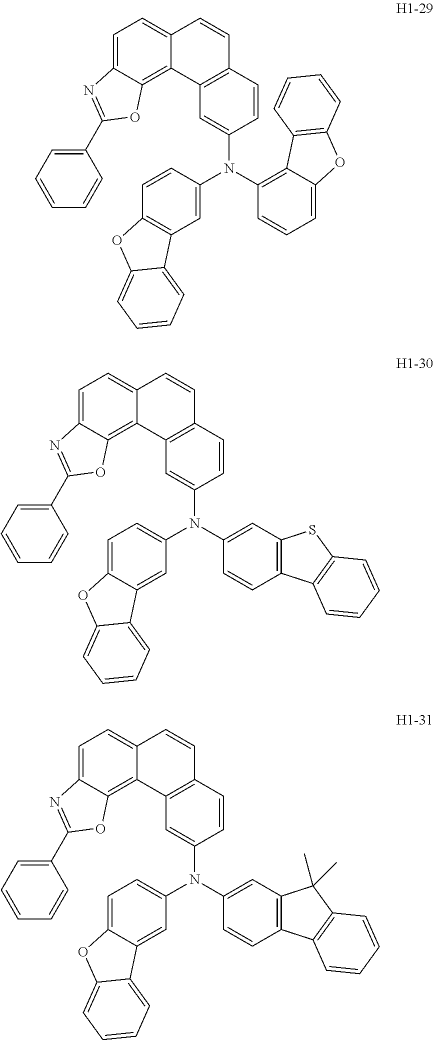

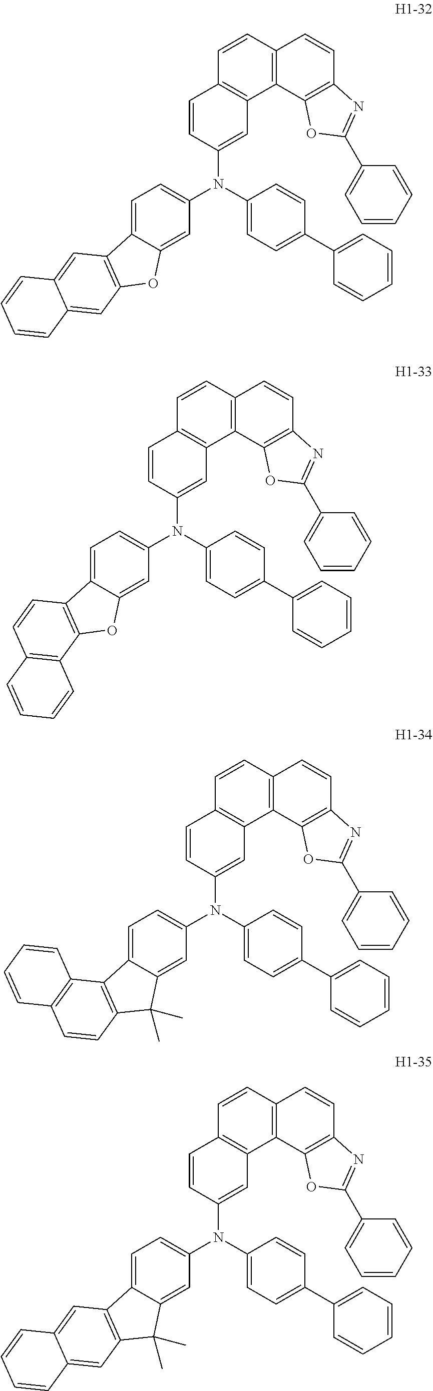 [SMILES: c1ccc(-c2ccc(N(c3ccc4c(c3)oc3cc5ccccc5cc34)c3ccc4ccc5ccc6nc(-c7ccccc7)oc6c5c4c3)cc2)cc1 ; c1ccc(-c2ccc(N(c3ccc4c(c3)oc3c5ccccc5ccc43)c3ccc4ccc5ccc6nc(-c7ccccc7)oc6c5c4c3)cc2)cc1 ; CC1(C)c2cc(N(c3ccc(-c4ccccc4)cc3)c3ccc4ccc5ccc6nc(-c7ccccc7)oc6c5c4c3)ccc2-c2c1ccc1ccccc21 ; CC1(C)c2cc(N(c3ccc(-c4ccccc4)cc3)c3ccc4ccc5ccc6nc(-c7ccccc7)oc6c5c4c3)ccc2-c2cc3ccccc3cc21]

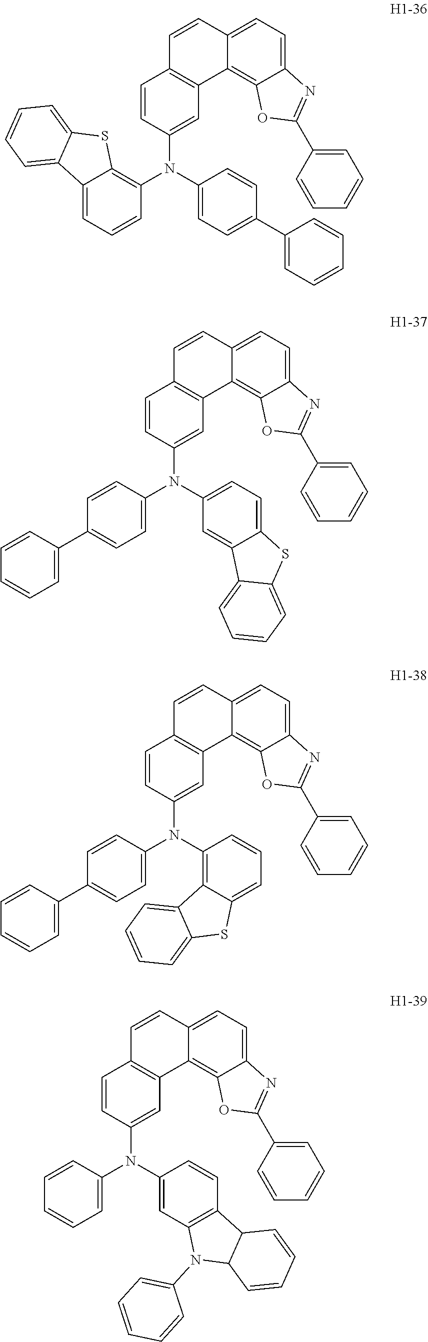

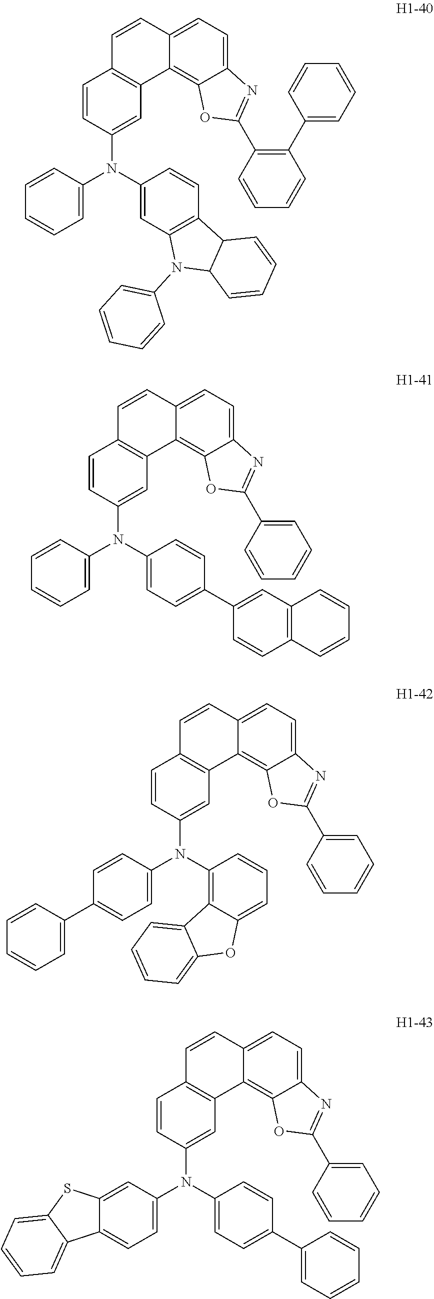

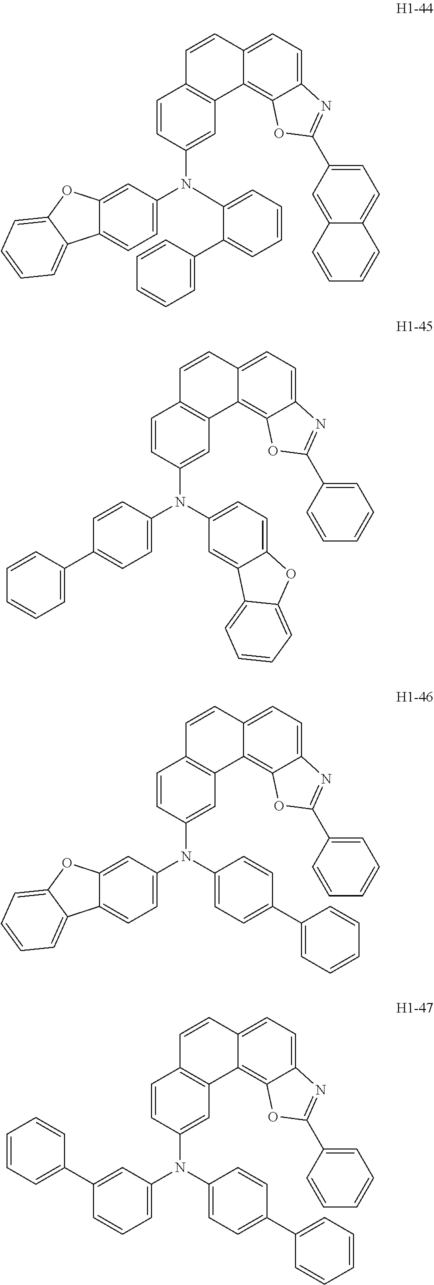

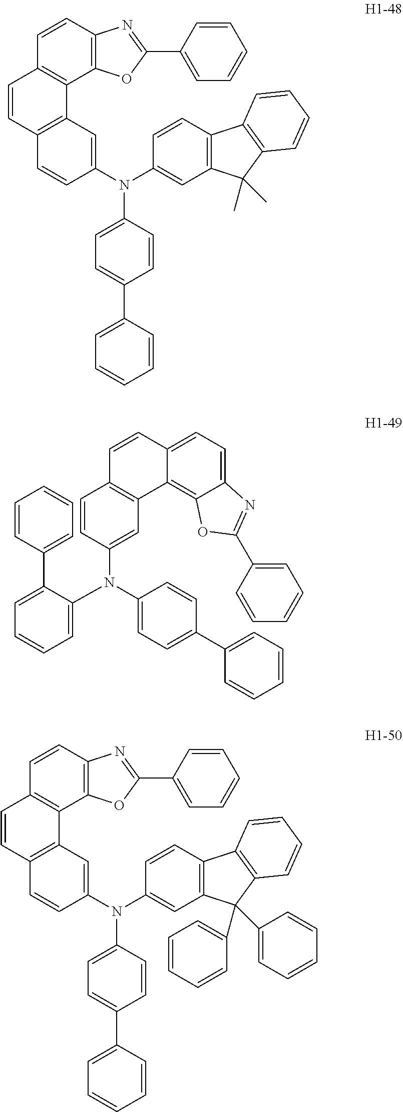 [SMILES: CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3ccc4ccc5ccc6nc(-c7ccccc7)oc6c5c4c3)c3ccccc3-c3ccccc3)cc2)cc1 ; c1ccc(-c2ccc(N(c3ccc4c(c3)C(c3ccccc3)(c3ccccc3)c3ccccc3-4)c3ccc4ccc5ccc6nc(-c7ccccc7)oc6c5c4c3)cc2)cc1]

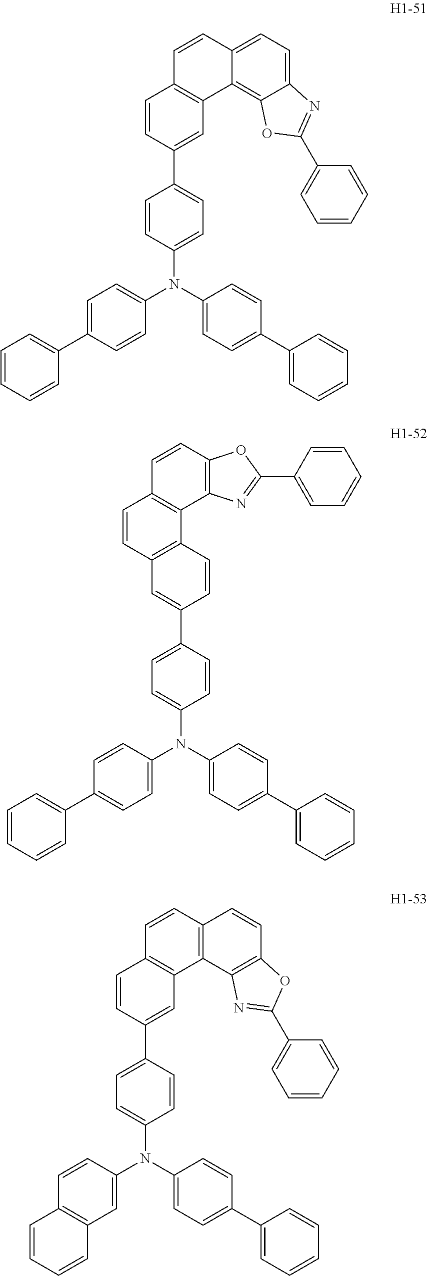 [SMILES: c1ccc(-c2ccc(N(c3ccc(-c4ccccc4)cc3)c3ccc(-c4ccc5ccc6ccc7nc(-c8ccccc8)oc7c6c5c4)cc3)cc2)cc1 ; c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1]

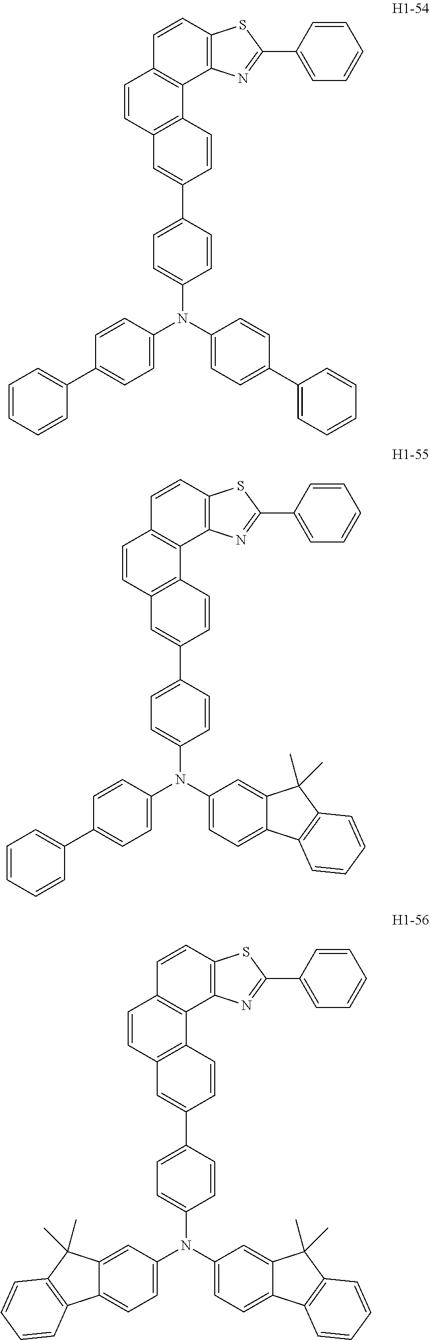 [SMILES: c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21]

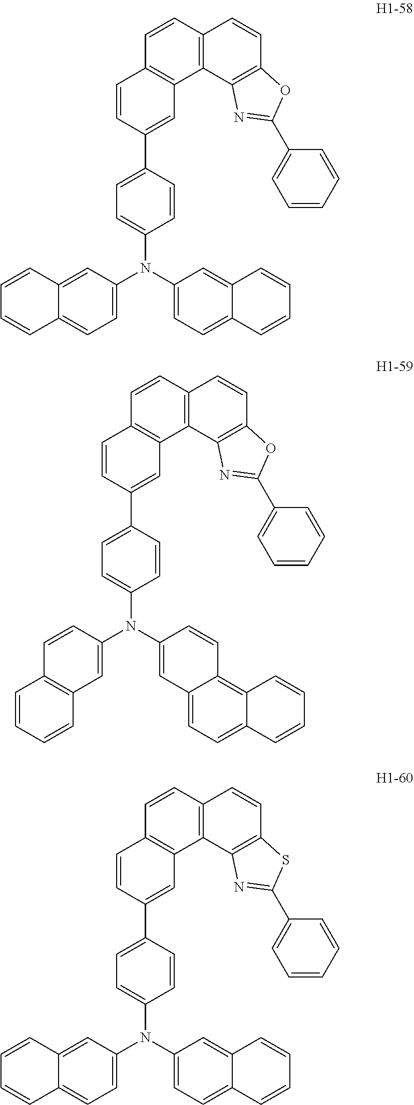

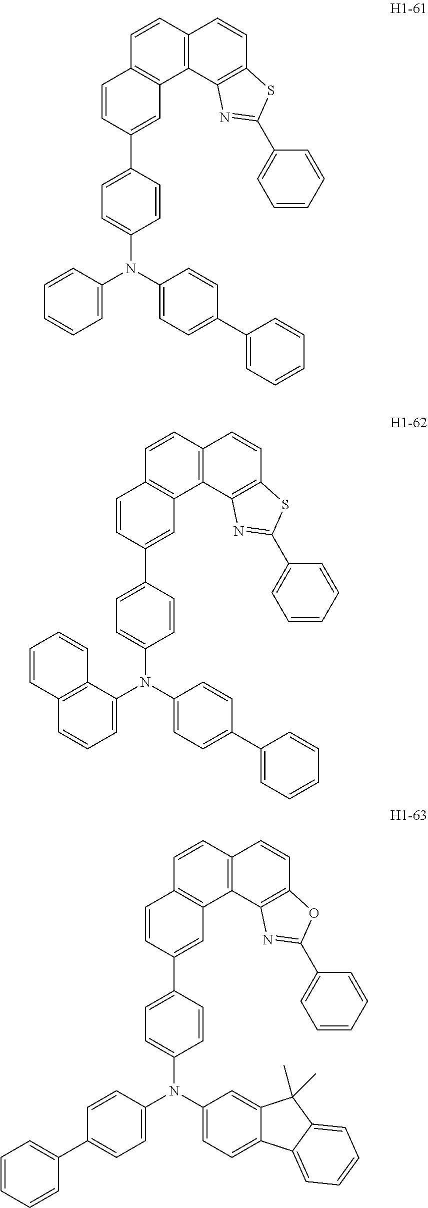 [SMILES: c1ccc(-c2ccc(N(c3ccccc3)c3ccc(-c4ccc5ccc6ccc7sc(-c8ccccc8)nc7c6c5c4)cc3)cc2)cc1 ; c1ccc(-c2ccc(N(c3ccc(-c4ccc5ccc6ccc7sc(-c8ccccc8)nc7c6c5c4)cc3)c3cccc4ccccc34)cc2)cc1 ; CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21]

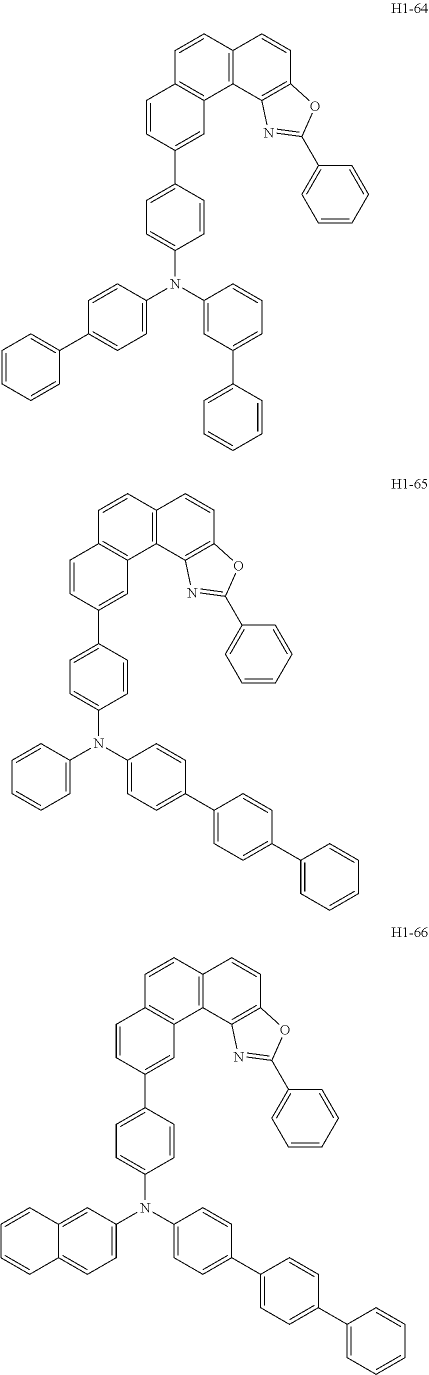 [SMILES: c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1]

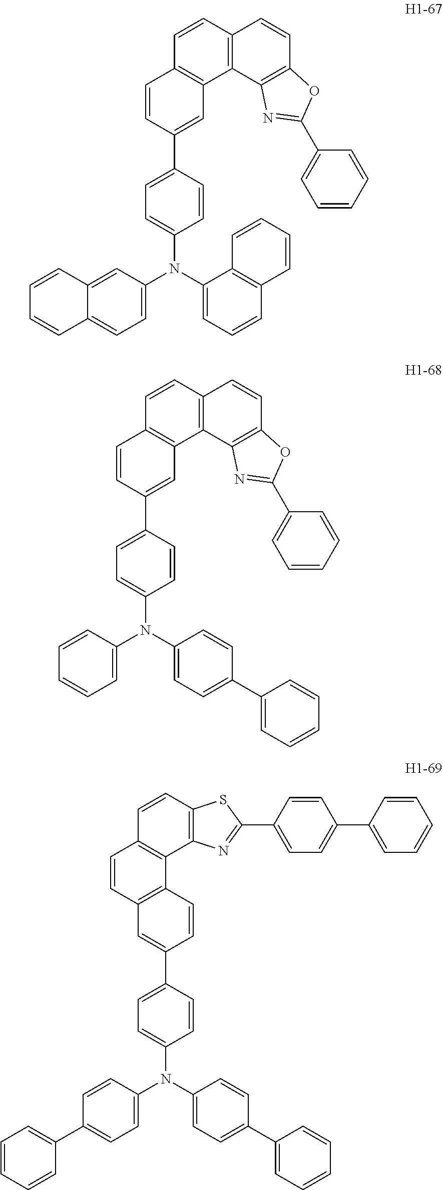

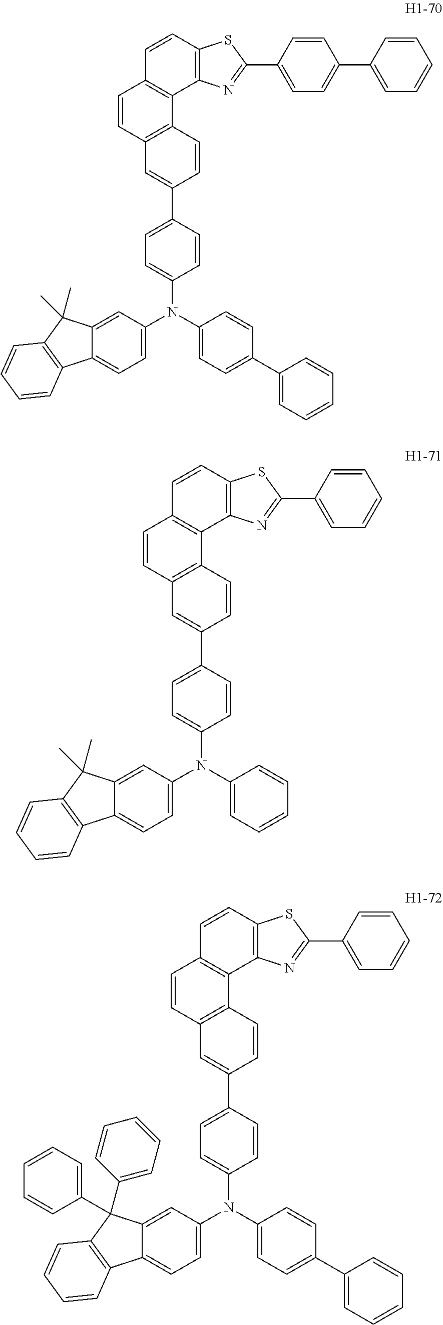 [SMILES: CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21 ; CC1(C)c2ccccc2-c2ccc(N(c3ccccc3)c3ccc(-c4ccc5c(ccc6ccc7sc(-c8ccccc8)nc7c65)c4)cc3)cc21 ; c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1]

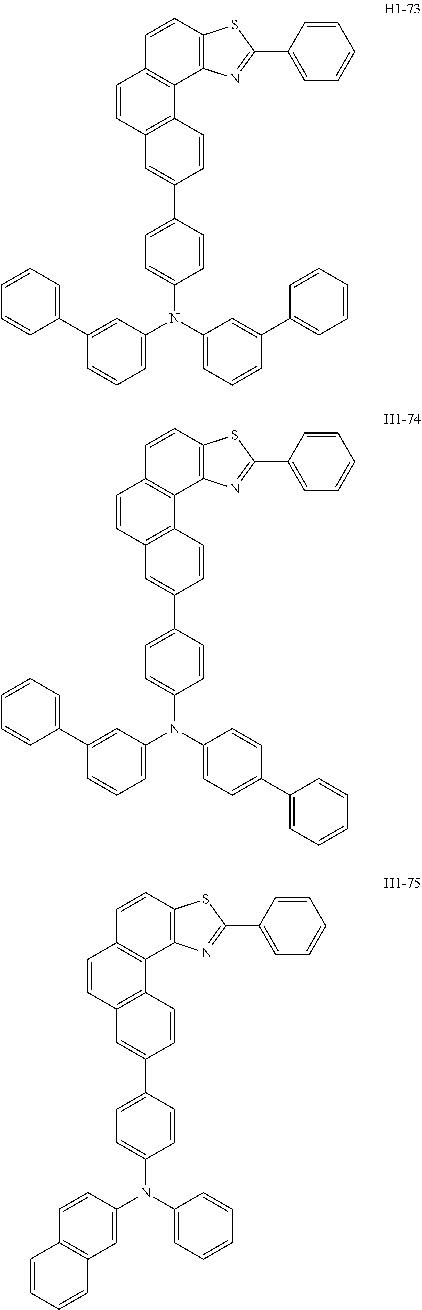 [SMILES: c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1 ; c1ccc(-c2nc3c(ccc4ccc5cc(-c6ccc(N(c7ccccc7)c7ccc8ccccc8c7)cc6)ccc5c43)s2)cc1]

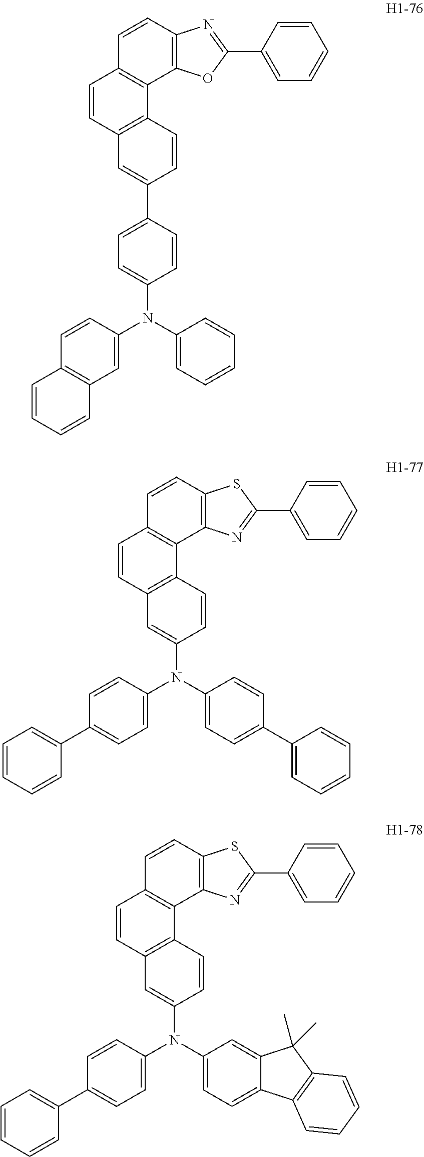 [SMILES: c1ccc(-c2nc3ccc4ccc5cc(-c6ccc(N(c7ccccc7)c7ccc8ccccc8c7)cc6)ccc5c4c3o2)cc1 ; c1ccc(-c2ccc(N(c3ccc(-c4ccccc4)cc3)c3ccc4c(ccc5ccc6sc(-c7ccccc7)nc6c54)c3)cc2)cc1 ; CC1(C)c2ccccc2-c2ccc(N(c3ccc(-c4ccccc4)cc3)c3ccc4c(ccc5ccc6sc(-c7ccccc7)nc6c54)c3)cc21]

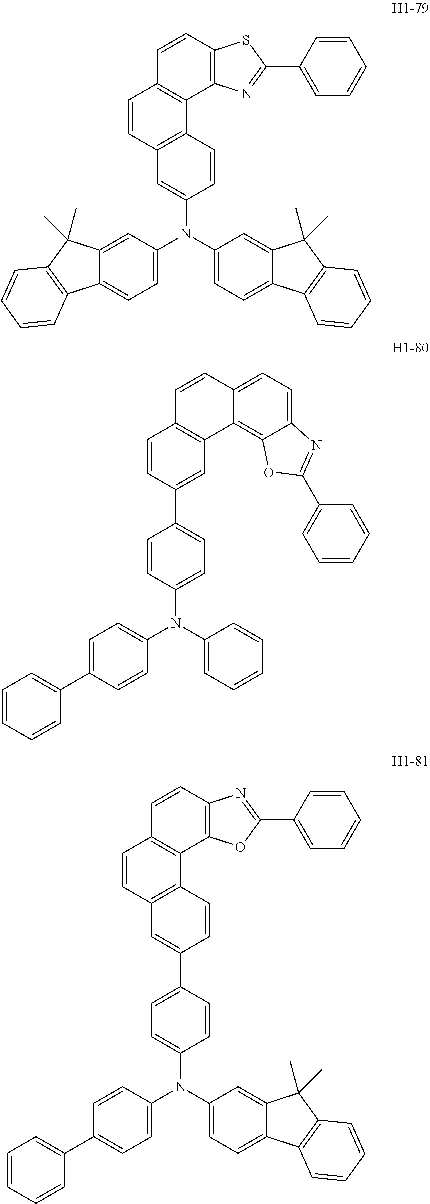 [SMILES: CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; c1ccc(-c2ccc(N(c3ccccc3)c3ccc(-c4ccc5ccc6ccc7nc(-c8ccccc8)oc7c6c5c4)cc3)cc2)cc1 ; CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21]

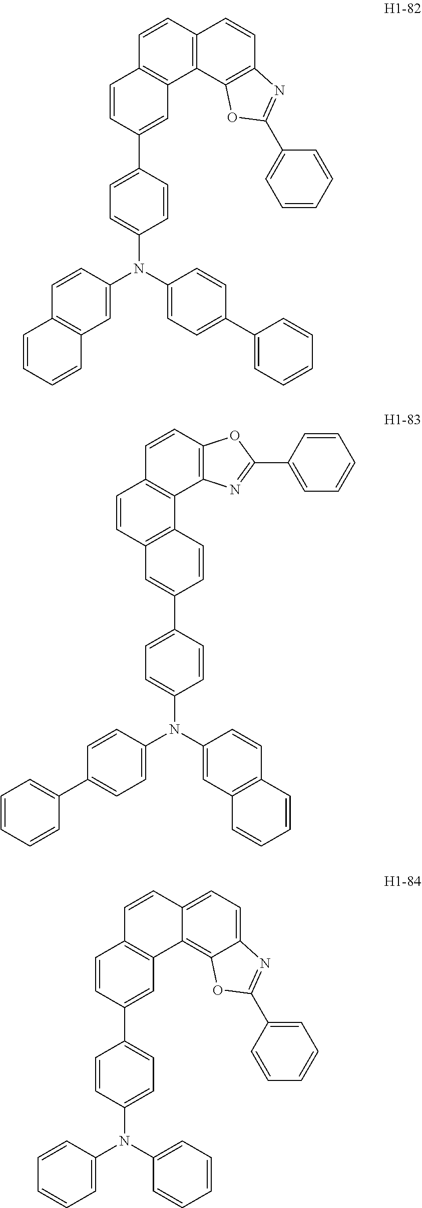 [SMILES: c1ccc(-c2ccc(N(c3ccc(-c4ccc5ccc6ccc7nc(-c8ccccc8)oc7c6c5c4)cc3)c3ccc4ccccc4c3)cc2)cc1 ; c1ccc(-c2ccc(N(c3ccc(-c4ccc5c(ccc6ccc7oc(-c8ccccc8)nc7c65)c4)cc3)c3ccc4ccccc4c3)cc2)cc1 ; c1ccc(-c2nc3ccc4ccc5ccc(-c6ccc(N(c7ccccc7)c7ccccc7)cc6)cc5c4c3o2)cc1]

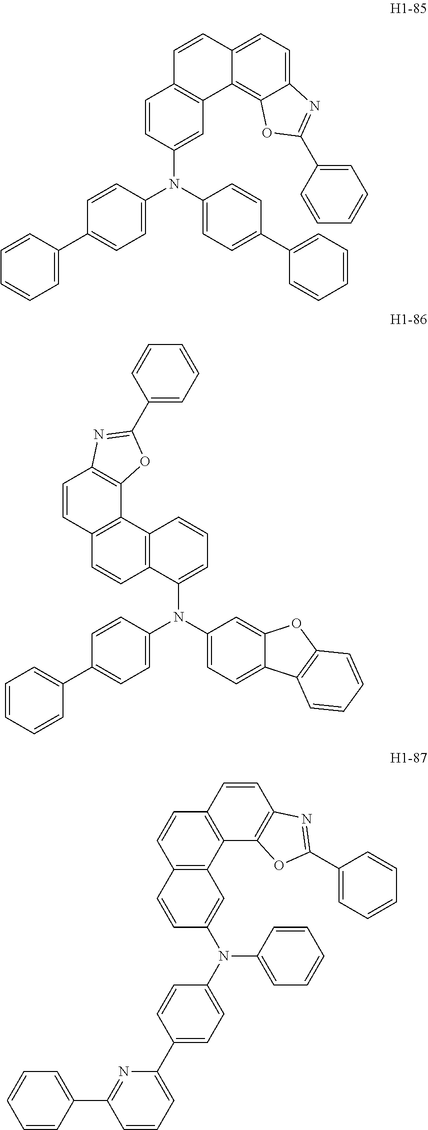 [SMILES: c1ccc(-c2ccc(N(c3ccc(-c4ccccc4)cc3)c3ccc4ccc5ccc6nc(-c7ccccc7)oc6c5c4c3)cc2)cc1 ; c1ccc(-c2ccc(N(c3ccc4c(c3)oc3ccccc34)c3cccc4c3ccc3ccc5nc(-c6ccccc6)oc5c34)cc2)cc1 ; c1ccc(-c2cccc(-c3ccc(N(c4ccccc4)c4ccc5ccc6ccc7nc(-c8ccccc8)oc7c6c5c4)cc3)n2)cc1]

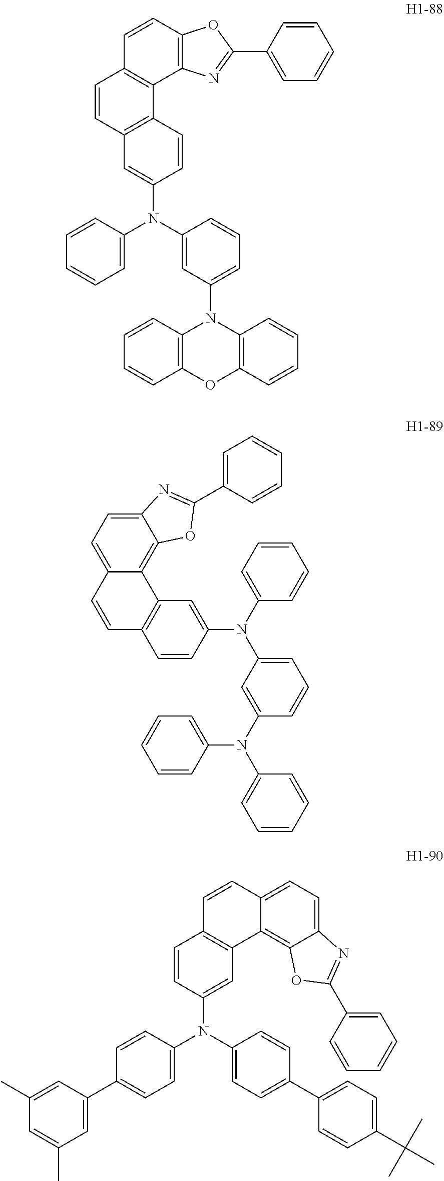 [SMILES: c1ccc(-c2nc3c(ccc4ccc5cc(N(c6ccccc6)c6cccc(N7c8ccccc8Oc8ccccc87)c6)ccc5c43)o2)cc1 ; c1ccc(-c2nc3ccc4ccc5ccc(N(c6ccccc6)c6cccc(N(c7ccccc7)c7ccccc7)c6)cc5c4c3o2)cc1 ; Cc1cc(C)cc(-c2ccc(N(c3ccc(-c4ccc(C(C)(C)C)cc4)cc3)c3ccc4ccc5ccc6nc(-c7ccccc7)oc6c5c4c3)cc2)c1]

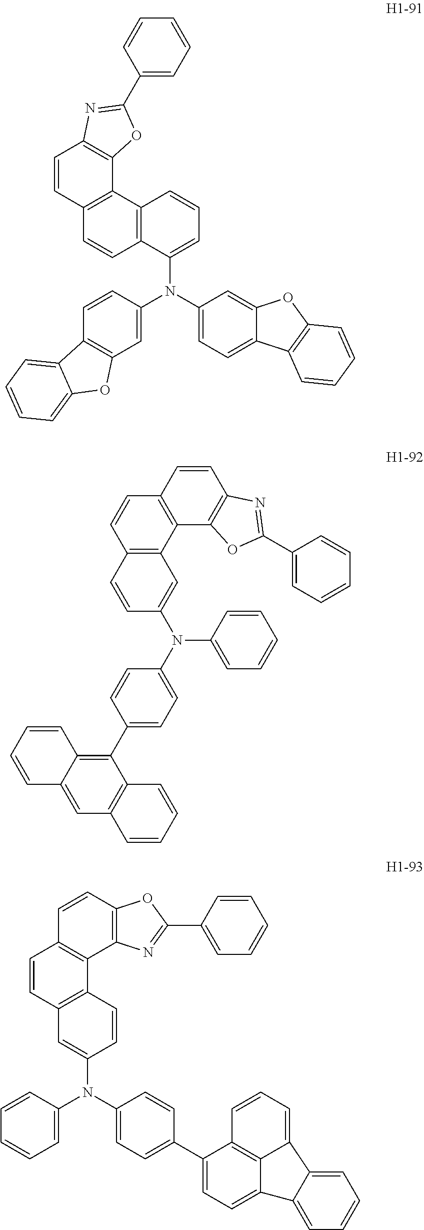 [SMILES: c1ccc(-c2nc3ccc4ccc5c(N(c6ccc7c(c6)oc6ccccc67)c6ccc7c(c6)oc6ccccc67)cccc5c4c3o2)cc1 ; c1ccc(-c2nc3ccc4ccc5ccc(N(c6ccccc6)c6ccc(-c7c8ccccc8cc8ccccc78)cc6)cc5c4c3o2)cc1 ; c1ccc(-c2nc3c(ccc4ccc5cc(N(c6ccccc6)c6ccc(-c7ccc8c9c(cccc79)-c7ccccc7-8)cc6)ccc5c43)o2)cc1]

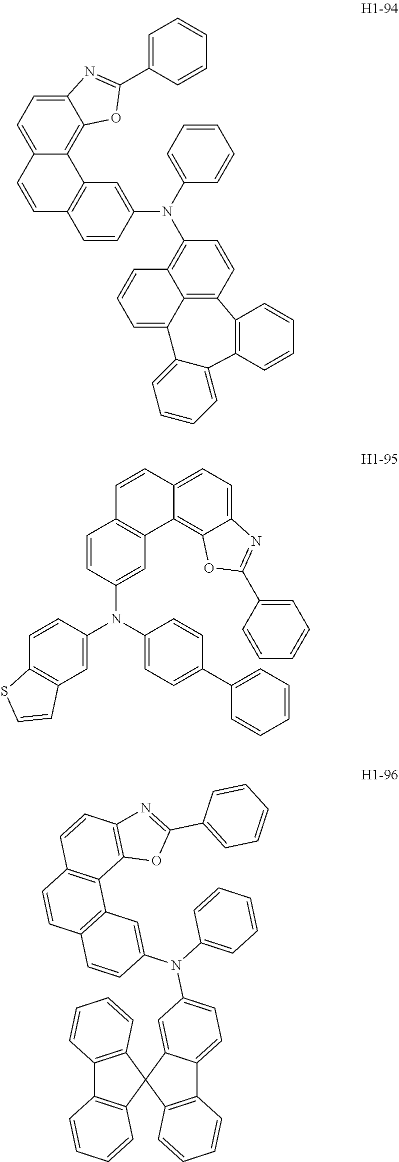

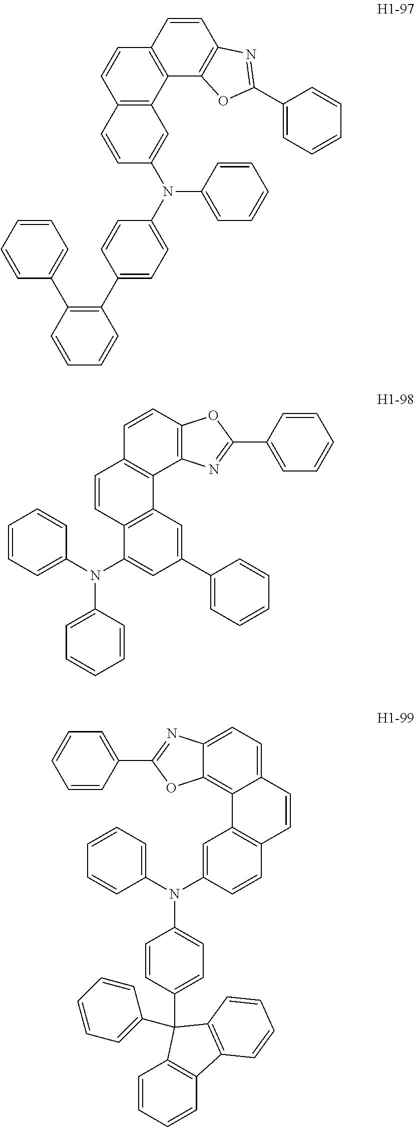 [SMILES: c1ccc(-c2nc3ccc4ccc5ccc(N(c6ccccc6)c6ccc(-c7ccccc7-c7ccccc7)cc6)cc5c4c3o2)cc1 ; c1ccc(-c2cc(N(c3ccccc3)c3ccccc3)c3ccc4ccc5oc(-c6ccccc6)nc5c4c3c2)cc1 ; c1ccc(-c2nc3ccc4ccc5ccc(N(c6ccccc6)c6ccc(C7(c8ccccc8)c8ccccc8-c8ccccc87)cc6)cc5c4c3o2)cc1]

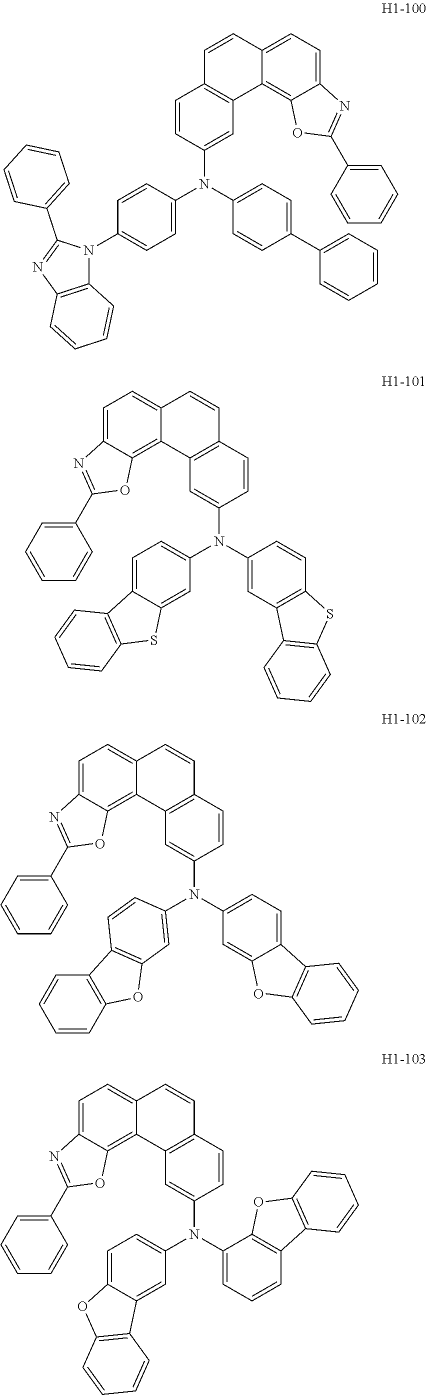 [SMILES: c1ccc(-c2ccc(N(c3ccc(-n4c(-c5ccccc5)nc5ccccc54)cc3)c3ccc4ccc5ccc6nc(-c7ccccc7)oc6c5c4c3)cc2)cc1 ; c1ccc(-c2nc3ccc4ccc5ccc(N(c6ccc7c(c6)sc6ccccc67)c6ccc7sc8ccccc8c7c6)cc5c4c3o2)cc1 ; c1ccc(-c2nc3ccc4ccc5ccc(N(c6ccc7c(c6)oc6ccccc67)c6ccc7c(c6)oc6ccccc67)cc5c4c3o2)cc1 ; c1ccc(-c2nc3ccc4ccc5ccc(N(c6ccc7oc8ccccc8c7c6)c6cccc7c6oc6ccccc67)cc5c4c3o2)cc1]

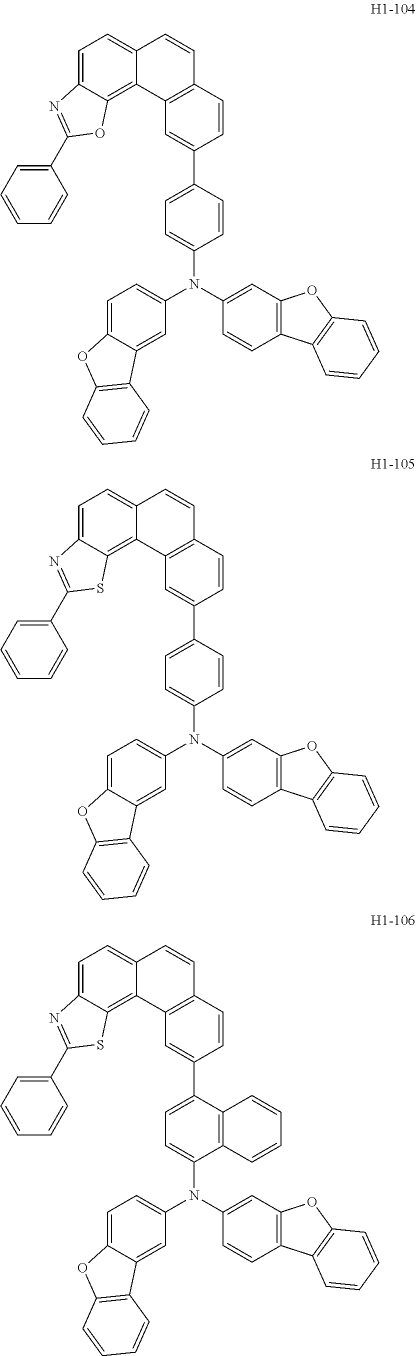

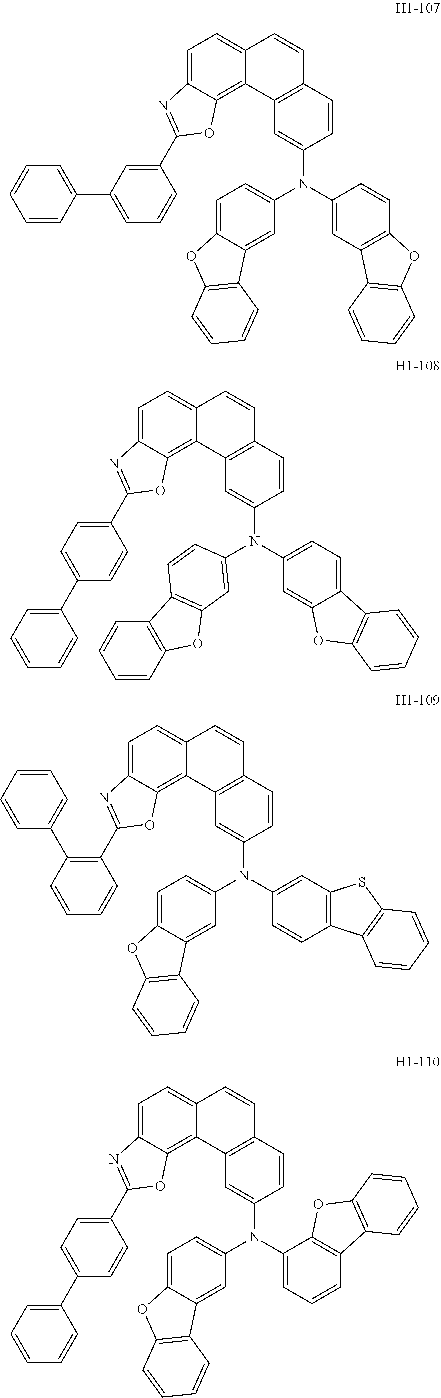 [SMILES: c1ccc(-c2cccc(-c3nc4ccc5ccc6ccc(N(c7ccc8oc9ccccc9c8c7)c7ccc8oc9ccccc9c8c7)cc6c5c4o3)c2)cc1 ; c1ccc(-c2ccc(-c3nc4ccc5ccc6ccc(N(c7ccc8c(c7)oc7ccccc78)c7ccc8c(c7)oc7ccccc78)cc6c5c4o3)cc2)cc1 ; c1ccc(-c2ccccc2-c2nc3ccc4ccc5ccc(N(c6ccc7c(c6)sc6ccccc67)c6ccc7oc8ccccc8c7c6)cc5c4c3o2)cc1 ; c1ccc(-c2ccc(-c3nc4ccc5ccc6ccc(N(c7ccc8oc9ccccc9c8c7)c7cccc8c7oc7ccccc78)cc6c5c4o3)cc2)cc1]

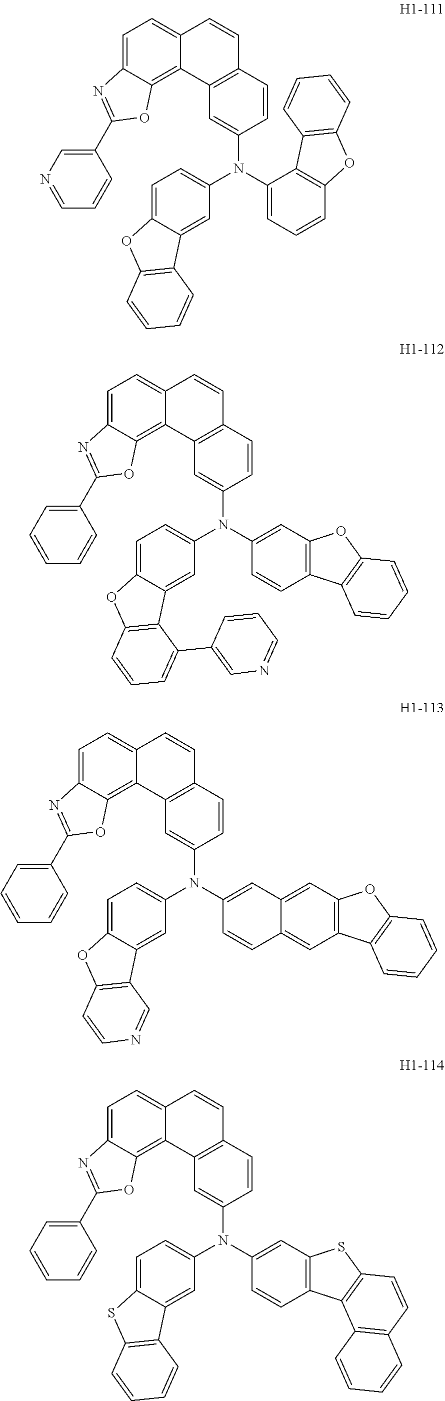 [SMILES: c1cncc(-c2nc3ccc4ccc5ccc(N(c6ccc7oc8ccccc8c7c6)c6cccc7oc8ccccc8c67)cc5c4c3o2)c1 ; c1ccc(-c2nc3ccc4ccc5ccc(N(c6ccc7c(c6)oc6ccccc67)c6ccc7oc8cccc(-c9cccnc9)c8c7c6)cc5c4c3o2)cc1 ; c1ccc(-c2nc3ccc4ccc5ccc(N(c6ccc7cc8c(cc7c6)oc6ccccc68)c6ccc7oc8ccncc8c7c6)cc5c4c3o2)cc1 ; c1ccc(-c2nc3ccc4ccc5ccc(N(c6ccc7c(c6)sc6ccc8ccccc8c67)c6ccc7sc8ccccc8c7c6)cc5c4c3o2)cc1]

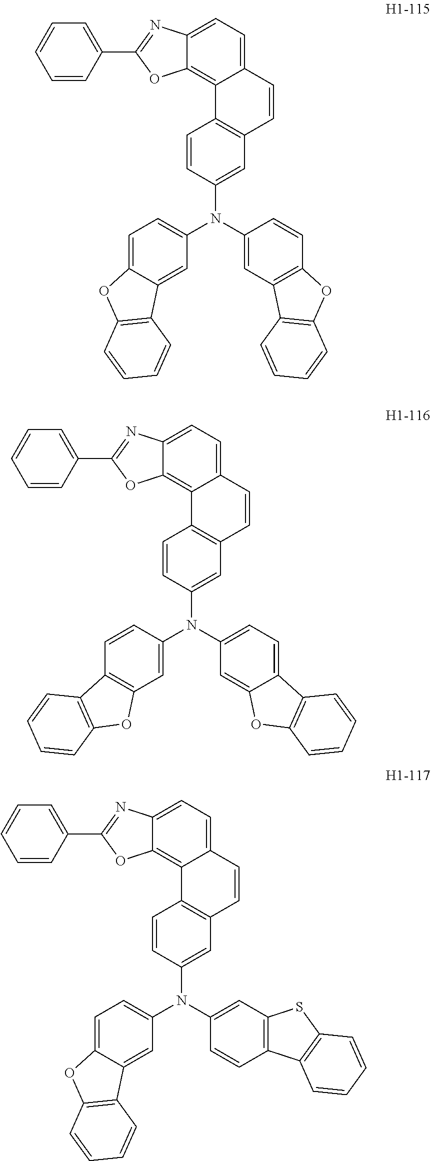 [SMILES: c1ccc(-c2nc3ccc4ccc5cc(N(c6ccc7oc8ccccc8c7c6)c6ccc7oc8ccccc8c7c6)ccc5c4c3o2)cc1 ; c1ccc(-c2nc3ccc4ccc5cc(N(c6ccc7c(c6)oc6ccccc67)c6ccc7c(c6)oc6ccccc67)ccc5c4c3o2)cc1 ; c1ccc(-c2nc3ccc4ccc5cc(N(c6ccc7c(c6)sc6ccccc67)c6ccc7oc8ccccc8c7c6)ccc5c4c3o2)cc1]

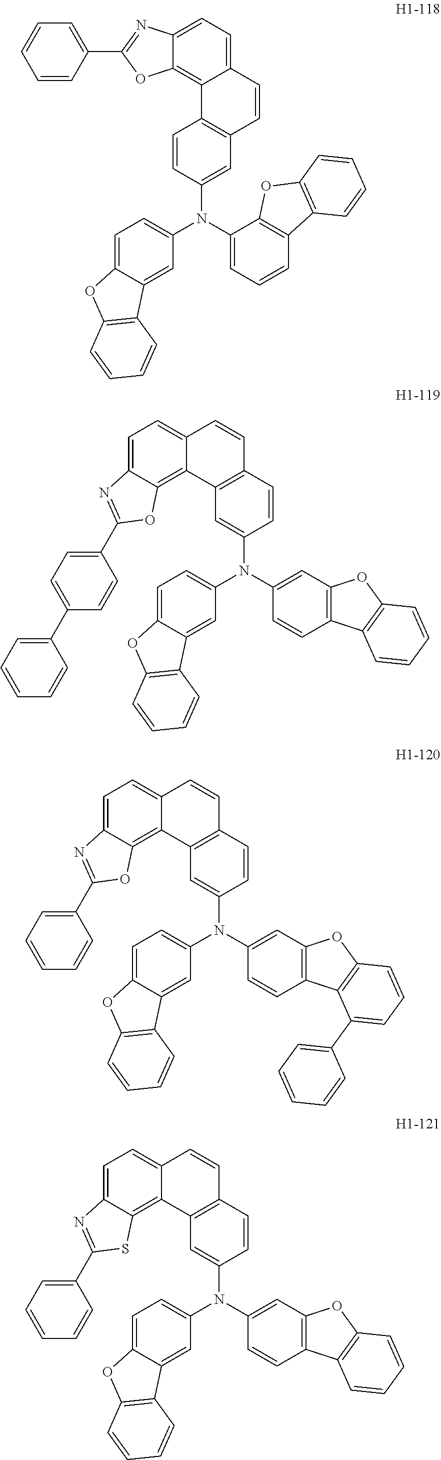 [SMILES: c1ccc(-c2nc3ccc4ccc5cc(N(c6ccc7oc8ccccc8c7c6)c6cccc7c6oc6ccccc67)ccc5c4c3o2)cc1 ; c1ccc(-c2ccc(-c3nc4ccc5ccc6ccc(N(c7ccc8c(c7)oc7ccccc78)c7ccc8oc9ccccc9c8c7)cc6c5c4o3)cc2)cc1 ; c1ccc(-c2nc3ccc4ccc5ccc(N(c6ccc7c(c6)oc6cccc(-c8ccccc8)c67)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3s2)cc1]

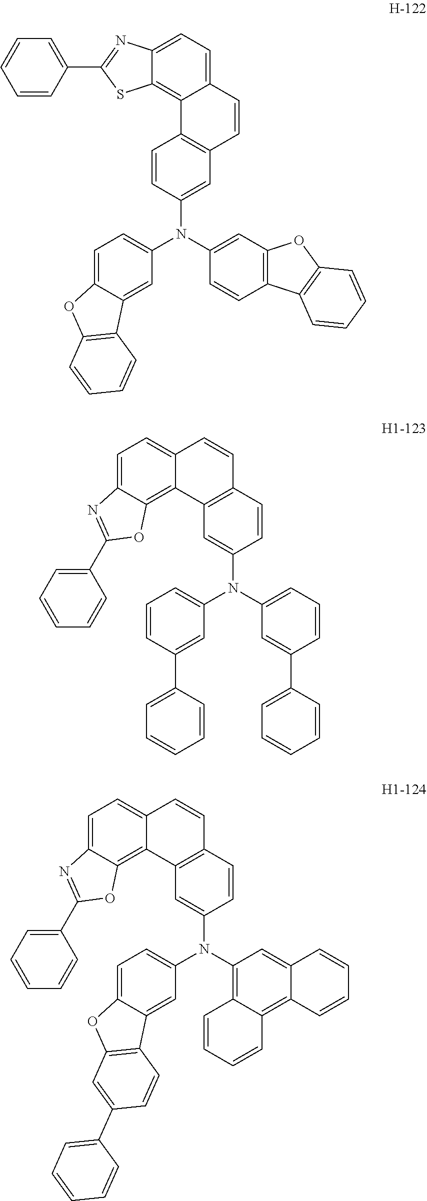

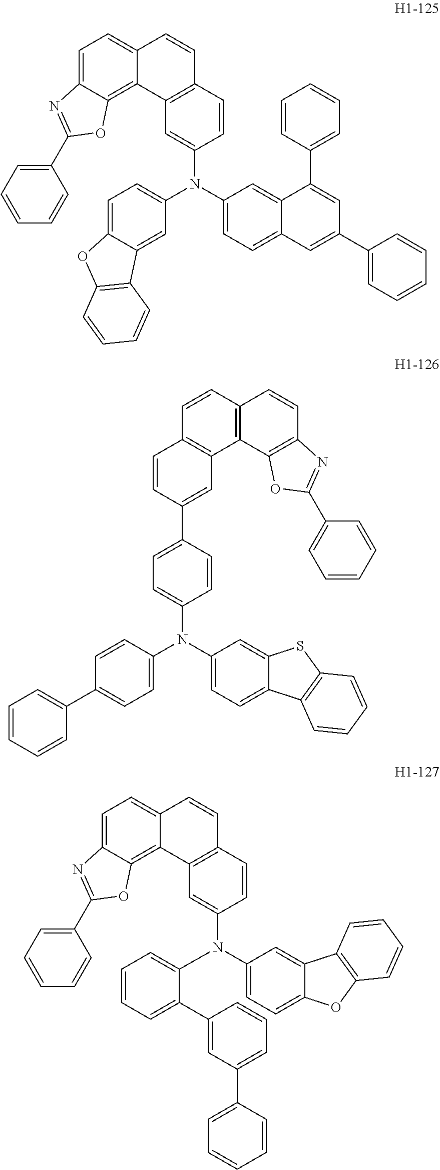 [SMILES: c1ccc(-c2cc(-c3ccccc3)c3cc(N(c4ccc5oc6ccccc6c5c4)c4ccc5ccc6ccc7nc(-c8ccccc8)oc7c6c5c4)ccc3c2)cc1 ; c1ccc(-c2ccc(N(c3ccc(-c4ccc5ccc6ccc7nc(-c8ccccc8)oc7c6c5c4)cc3)c3ccc4c(c3)sc3ccccc34)cc2)cc1 ; c1ccc(-c2cccc(-c3ccccc3N(c3ccc4oc5ccccc5c4c3)c3ccc4ccc5ccc6nc(-c7ccccc7)oc6c5c4c3)c2)cc1]

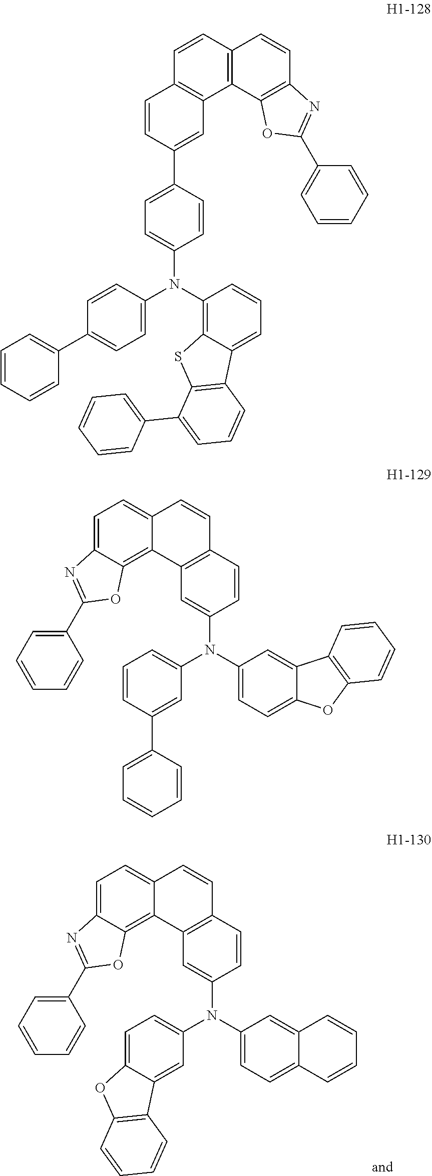 [SMILES: c1ccc(-c2ccc(N(c3ccc(-c4ccc5ccc6ccc7nc(-c8ccccc8)oc7c6c5c4)cc3)c3cccc4c3sc3c(-c5ccccc5)cccc34)cc2)cc1 ; c1ccc(-c2cccc(N(c3ccc4oc5ccccc5c4c3)c3ccc4ccc5ccc6nc(-c7ccccc7)oc6c5c4c3)c2)cc1 ; c1ccc(-c2nc3ccc4ccc5ccc(N(c6ccc7ccccc7c6)c6ccc7oc8ccccc8c7c6)cc5c4c3o2)cc1]

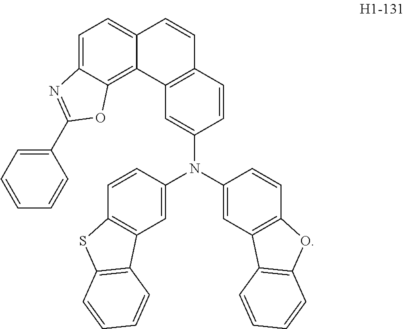 [SMILES: c1ccc(-c2nc3ccc4ccc5ccc(N(c6ccc7oc8ccccc8c7c6)c6ccc7sc8ccccc8c7c6)cc5c4c3o2)cc1]

9. The plurality of host materials according to claim 6, wherein the second host material comprises a compound the compound represented by the formula 3: 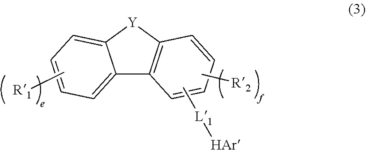
wherein
Y represents —O— or —S—;
L₁ represents a single bond, a substituted or unsubstituted (C6-C30)arylene, or a substituted or unsubstituted (3- to 30-membered)heteroarylene;
HAr′ represents a substituted or unsubstituted (3- to 30-membered)heteroaryl comprising at least one nitrogen;
R′₁ and R′₂ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or may be linked to the adjacent substituent to form a ring(s); and
e represents an integer 1 to 4, and f represents an integer 1 to 3; and when e and f are an integer of 2 or more, each of R′₁ and each of R′₂ may be the same or different.

10. The plurality of host materials according to claim 9, wherein the compound represented by the formula 3 is selected from the following compounds:

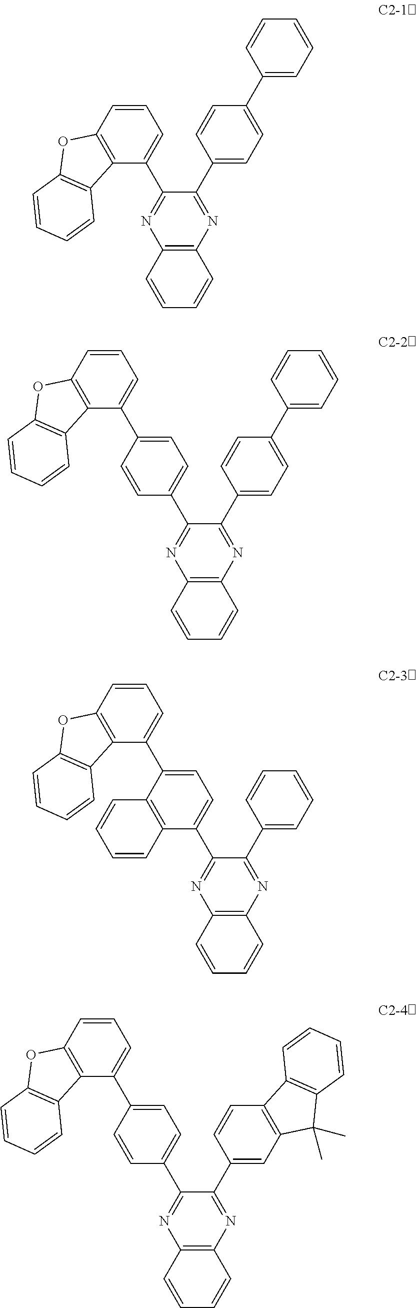 [SMILES: c1ccc(-c2ccc(-c3nc4ccccc4nc3-c3cccc4oc5ccccc5c34)cc2)cc1 ; c1ccc(-c2ccc(-c3nc4ccccc4nc3-c3ccc(-c4cccc5oc6ccccc6c45)cc3)cc2)cc1 ; c1ccc(-c2nc3ccccc3nc2-c2ccc(-c3cccc4oc5ccccc5c34)c3ccccc23)cc1 ; CC1(C)c2ccccc2-c2ccc(-c3nc4ccccc4nc3-c3ccc(-c4cccc5oc6ccccc6c45)cc3)cc21]

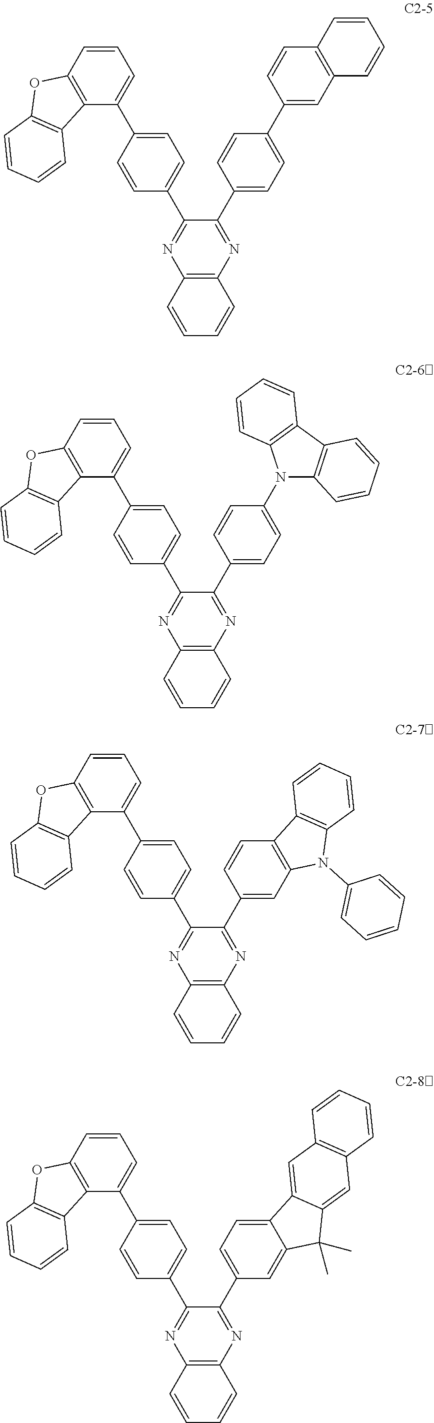 [SMILES: c1ccc2cc(-c3ccc(-c4nc5ccccc5nc4-c4ccc(-c5cccc6oc7ccccc7c56)cc4)cc3)ccc2c1 ; c1ccc2nc(-c3ccc(-n4c5ccccc5c5ccccc54)cc3)c(-c3ccc(-c4cccc5oc6ccccc6c45)cc3)nc2c1 ; c1ccc(-n2c3ccccc3c3ccc(-c4nc5ccccc5nc4-c4ccc(-c5cccc6oc7ccccc7c56)cc4)cc32)cc1 ; CC1(C)c2cc(-c3nc4ccccc4nc3-c3ccc(-c4cccc5oc6ccccc6c45)cc3)ccc2-c2cc3ccccc3cc21]

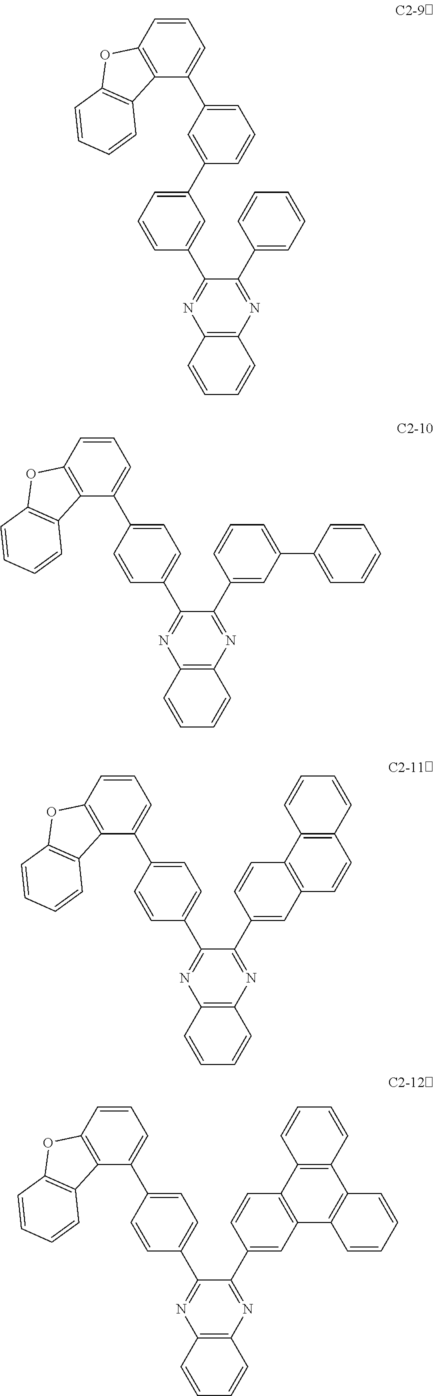 [SMILES: c1ccc(-c2nc3ccccc3nc2-c2cccc(-c3cccc(-c4cccc5oc6ccccc6c45)c3)c2)cc1 ; c1ccc(-c2cccc(-c3nc4ccccc4nc3-c3ccc(-c4cccc5oc6ccccc6c45)cc3)c2)cc1 ; c1ccc2c(c1)ccc1cc(-c3nc4ccccc4nc3-c3ccc(-c4cccc5oc6ccccc6c45)cc3)ccc12 ; c1ccc2nc(-c3ccc4c5ccccc5c5ccccc5c4c3)c(-c3ccc(-c4cccc5oc6ccccc6c45)cc3)nc2c1]

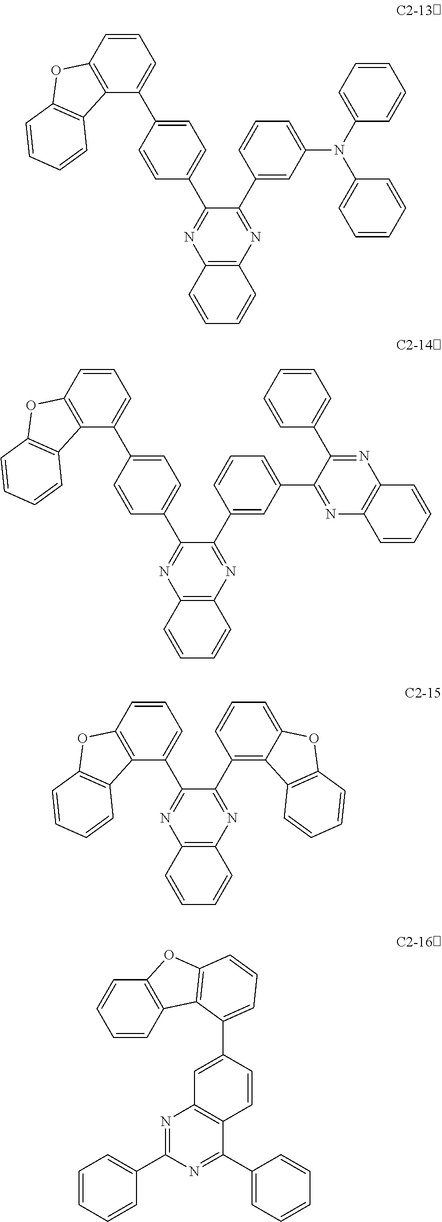 [SMILES: c1ccc(N(c2ccccc2)c2cccc(-c3nc4ccccc4nc3-c3ccc(-c4cccc5oc6ccccc6c45)cc3)c2)cc1 ; c1ccc(-c2nc3ccccc3nc2-c2cccc(-c3nc4ccccc4nc3-c3ccc(-c4cccc5oc6ccccc6c45)cc3)c2)cc1 ; c1ccc2nc(-c3cccc4oc5ccccc5c34)c(-c3cccc4oc5ccccc5c34)nc2c1 ; c1ccc(-c2nc(-c3ccccc3)c3ccc(-c4cccc5oc6ccccc6c45)cc3n2)cc1]

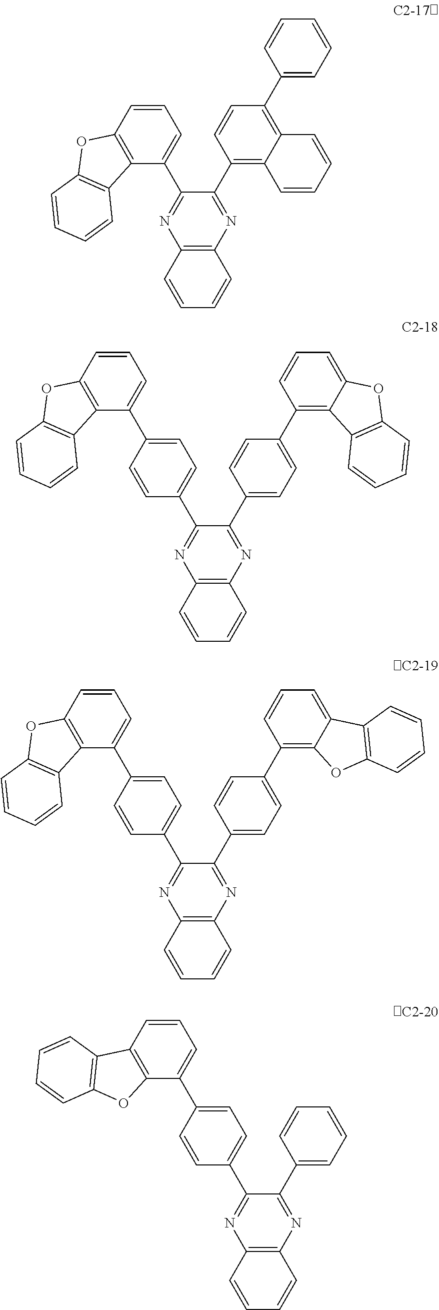

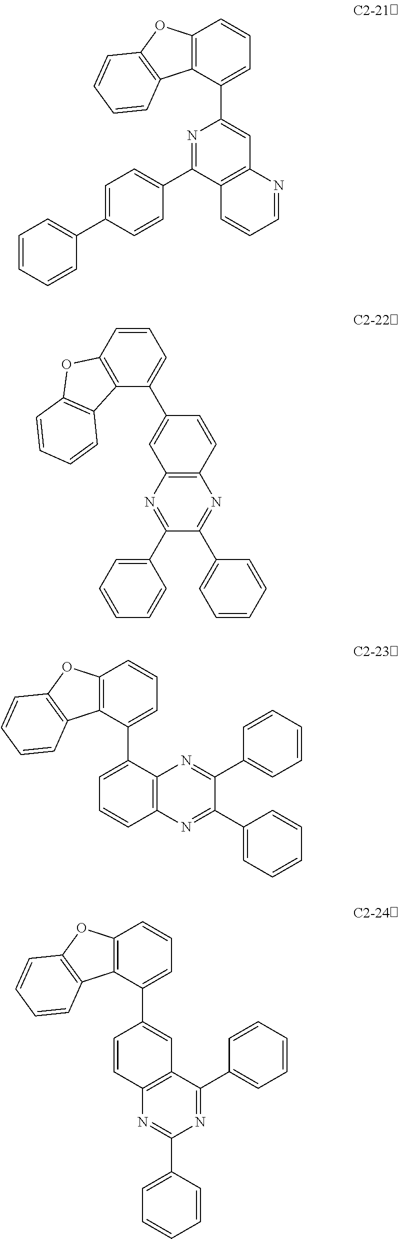

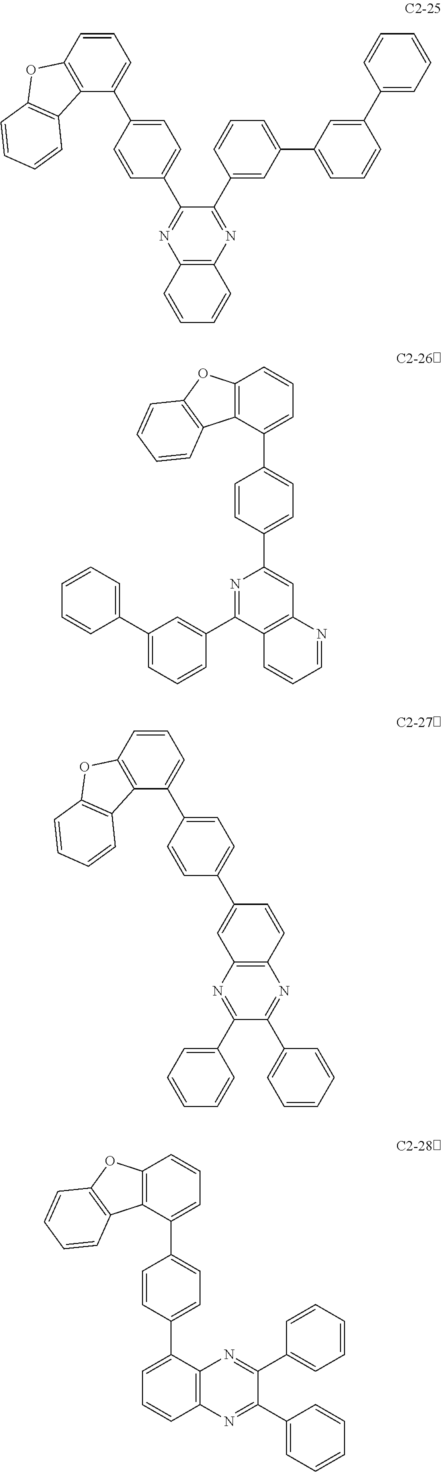 [SMILES: c1ccc(-c2cccc(-c3cccc(-c4nc5ccccc5nc4-c4ccc(-c5cccc6oc7ccccc7c56)cc4)c3)c2)cc1 ; c1ccc(-c2cccc(-c3nc(-c4ccc(-c5cccc6oc7ccccc7c56)cc4)cc4ncccc34)c2)cc1 ; c1ccc(-c2nc3ccc(-c4ccc(-c5cccc6oc7ccccc7c56)cc4)cc3nc2-c2ccccc2)cc1 ; c1ccc(-c2nc3cccc(-c4ccc(-c5cccc6oc7ccccc7c56)cc4)c3nc2-c2ccccc2)cc1]

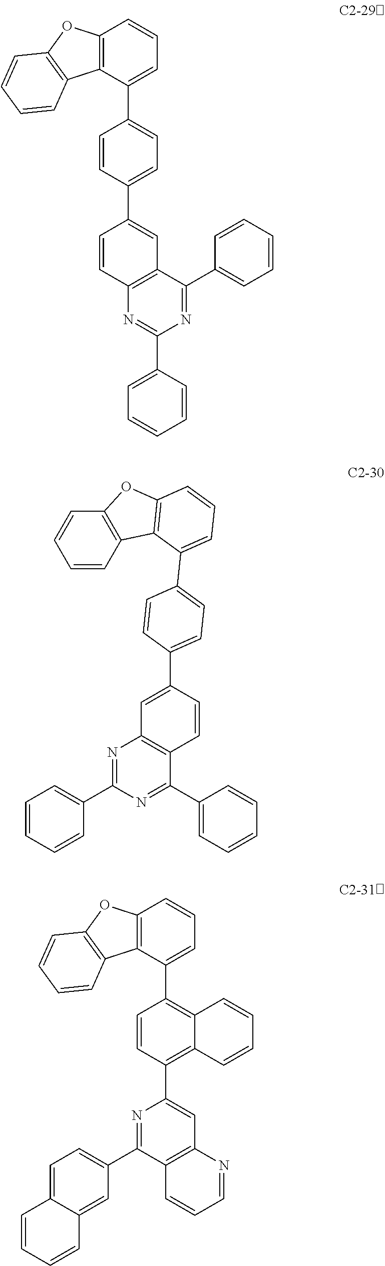 [SMILES: c1ccc(-c2nc(-c3ccccc3)c3cc(-c4ccc(-c5cccc6oc7ccccc7c56)cc4)ccc3n2)cc1 ; c1ccc(-c2nc(-c3ccccc3)c3ccc(-c4ccc(-c5cccc6oc7ccccc7c56)cc4)cc3n2)cc1 ; c1ccc2cc(-c3nc(-c4ccc(-c5cccc6oc7ccccc7c56)c5ccccc45)cc4ncccc34)ccc2c1]

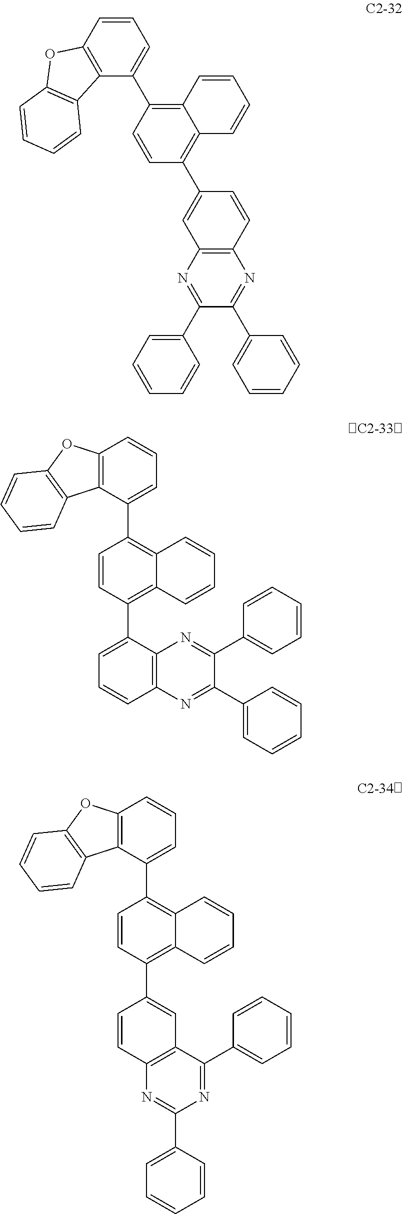 [SMILES: c1ccc(-c2nc3ccc(-c4ccc(-c5cccc6oc7ccccc7c56)c5ccccc45)cc3nc2-c2ccccc2)cc1 ; c1ccc(-c2nc3cccc(-c4ccc(-c5cccc6oc7ccccc7c56)c5ccccc45)c3nc2-c2ccccc2)cc1 ; c1ccc(-c2nc(-c3ccccc3)c3cc(-c4ccc(-c5cccc6oc7ccccc7c56)c5ccccc45)ccc3n2)cc1]

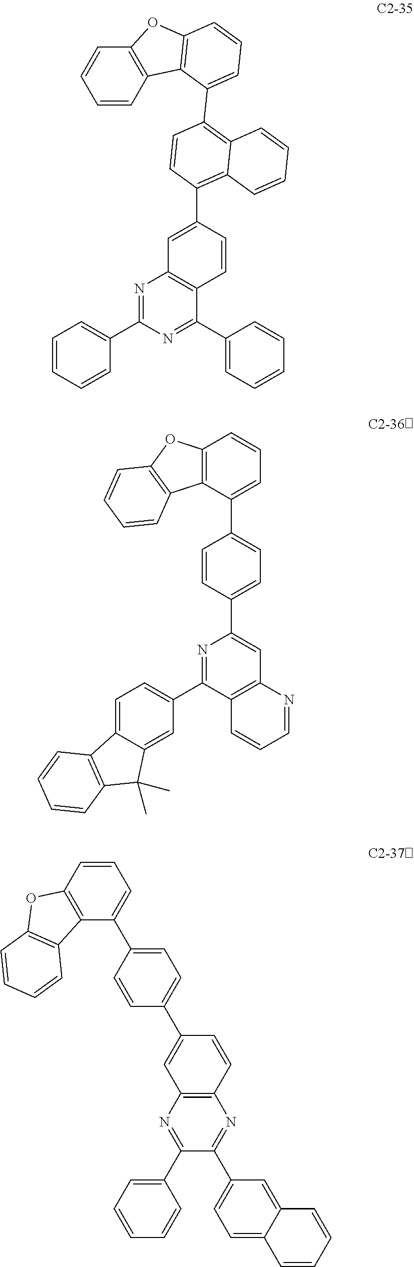 [SMILES: c1ccc(-c2nc(-c3ccccc3)c3ccc(-c4ccc(-c5cccc6oc7ccccc7c56)c5ccccc45)cc3n2)cc1 ; CC1(C)c2ccccc2-c2ccc(-c3nc(-c4ccc(-c5cccc6oc7ccccc7c56)cc4)cc4ncccc34)cc21 ; c1ccc(-c2nc3cc(-c4ccc(-c5cccc6oc7ccccc7c56)cc4)ccc3nc2-c2ccc3ccccc3c2)cc1]

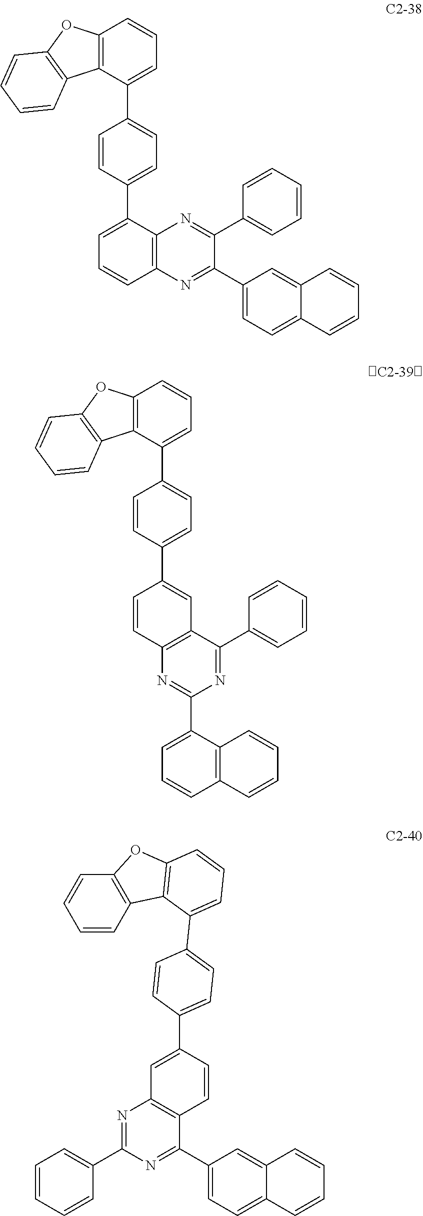 [SMILES: c1ccc(-c2nc3c(-c4ccc(-c5cccc6oc7ccccc7c56)cc4)cccc3nc2-c2ccc3ccccc3c2)cc1 ; c1ccc(-c2nc(-c3cccc4ccccc34)nc3ccc(-c4ccc(-c5cccc6oc7ccccc7c56)cc4)cc23)cc1 ; c1ccc(-c2nc(-c3ccc4ccccc4c3)c3ccc(-c4ccc(-c5cccc6oc7ccccc7c56)cc4)cc3n2)cc1]

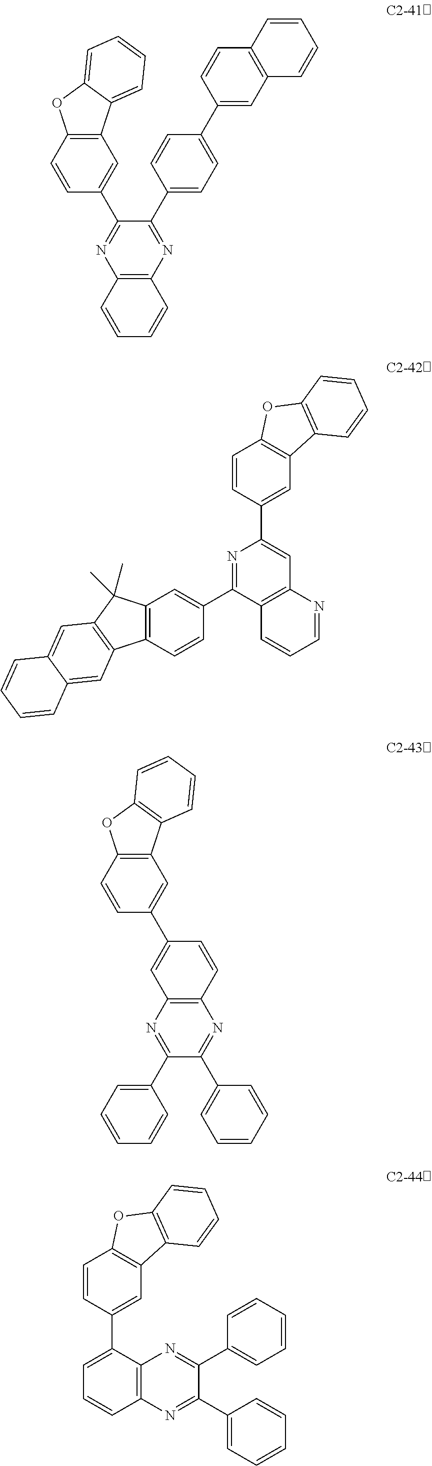 [SMILES: c1ccc2cc(-c3ccc(-c4nc5ccccc5nc4-c4ccc5oc6ccccc6c5c4)cc3)ccc2c1 ; CC1(C)c2cc(-c3nc(-c4ccc5oc6ccccc6c5c4)cc4ncccc34)ccc2-c2cc3ccccc3cc21 ; c1ccc(-c2nc3ccc(-c4ccc5oc6ccccc6c5c4)cc3nc2-c2ccccc2)cc1 ; c1ccc(-c2nc3cccc(-c4ccc5oc6ccccc6c5c4)c3nc2-c2ccccc2)cc1]

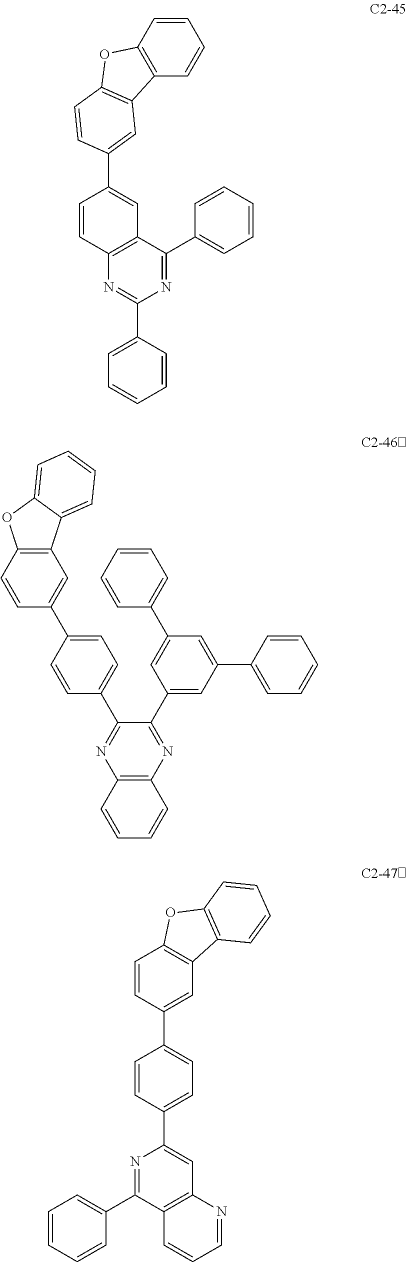 [SMILES: c1ccc(-c2nc(-c3ccccc3)c3cc(-c4ccc5oc6ccccc6c5c4)ccc3n2)cc1 ; c1ccc(-c2cc(-c3ccccc3)cc(-c3nc4ccccc4nc3-c3ccc(-c4ccc5oc6ccccc6c5c4)cc3)c2)cc1 ; c1ccc(-c2nc(-c3ccc(-c4ccc5oc6ccccc6c5c4)cc3)cc3ncccc23)cc1]

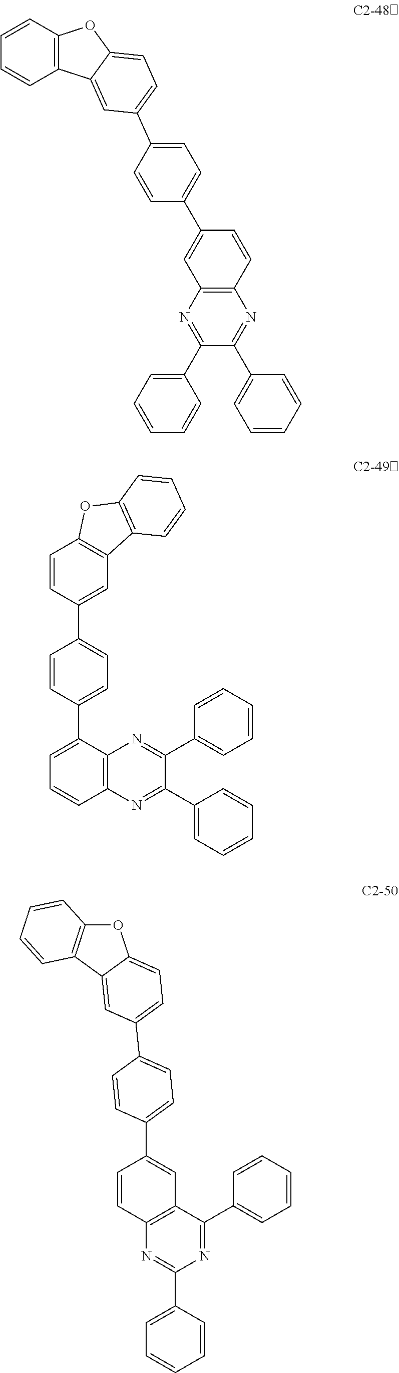

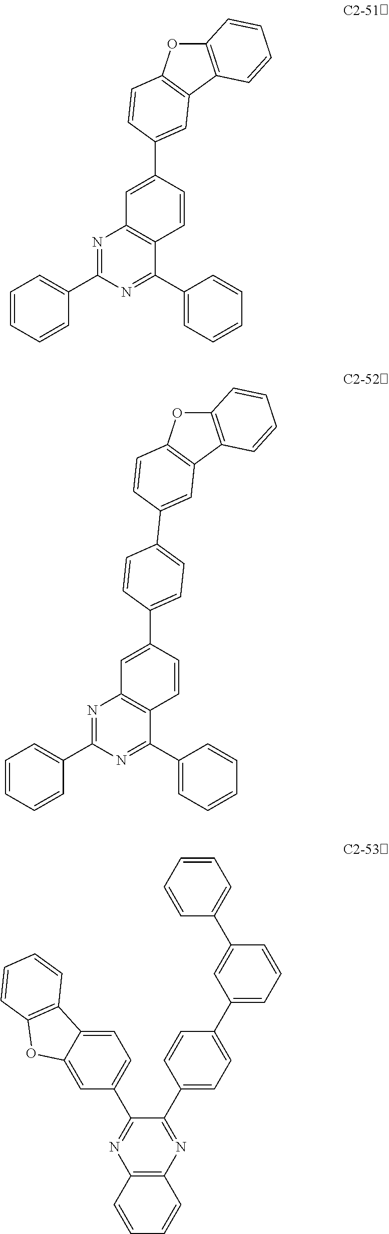 [SMILES: c1ccc(-c2nc(-c3ccccc3)c3ccc(-c4ccc5oc6ccccc6c5c4)cc3n2)cc1 ; c1ccc(-c2nc(-c3ccccc3)c3ccc(-c4ccc(-c5ccc6oc7ccccc7c6c5)cc4)cc3n2)cc1 ; c1ccc(-c2cccc(-c3ccc(-c4nc5ccccc5nc4-c4ccc5c(c4)oc4ccccc45)cc3)c2)cc1]

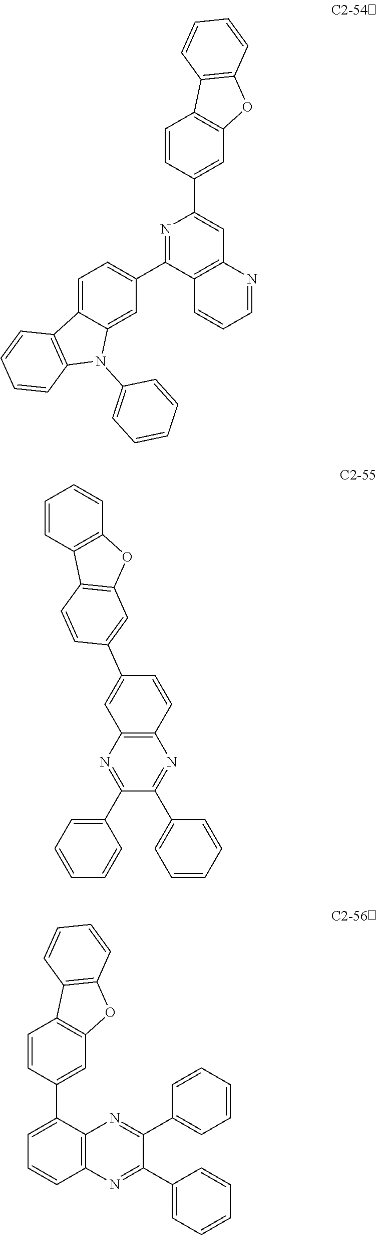 [SMILES: c1ccc(-n2c3ccccc3c3ccc(-c4nc(-c5ccc6c(c5)oc5ccccc56)cc5ncccc45)cc32)cc1 ; c1ccc(-c2nc3ccc(-c4ccc5c(c4)oc4ccccc45)cc3nc2-c2ccccc2)cc1 ; c1ccc(-c2nc3cccc(-c4ccc5c(c4)oc4ccccc45)c3nc2-c2ccccc2)cc1]

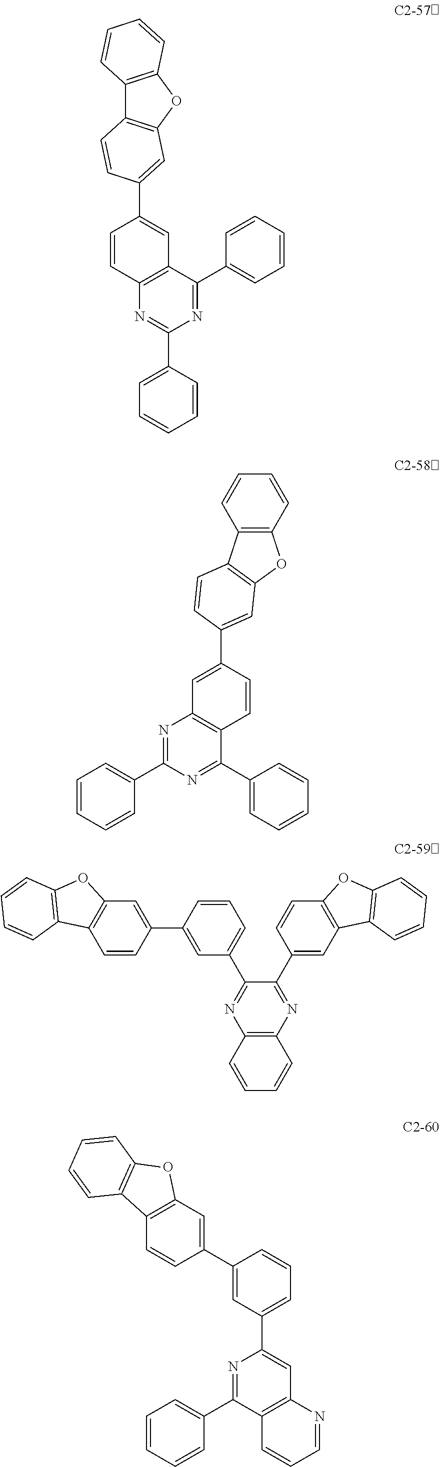 [SMILES: c1ccc(-c2nc(-c3ccccc3)c3cc(-c4ccc5c(c4)oc4ccccc45)ccc3n2)cc1 ; c1ccc(-c2nc(-c3ccccc3)c3ccc(-c4ccc5c(c4)oc4ccccc45)cc3n2)cc1 ; c1cc(-c2ccc3c(c2)oc2ccccc23)cc(-c2nc3ccccc3nc2-c2ccc3oc4ccccc4c3c2)c1 ; c1ccc(-c2nc(-c3cccc(-c4ccc5c(c4)oc4ccccc45)c3)cc3ncccc23)cc1]

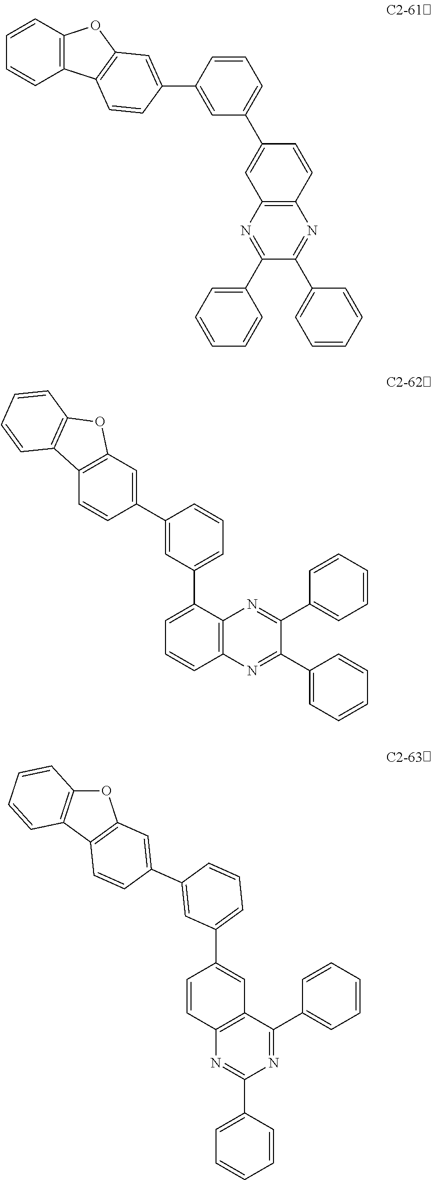 [SMILES: c1ccc(-c2nc3ccc(-c4cccc(-c5ccc6c(c5)oc5ccccc56)c4)cc3nc2-c2ccccc2)cc1 ; c1ccc(-c2nc3cccc(-c4cccc(-c5ccc6c(c5)oc5ccccc56)c4)c3nc2-c2ccccc2)cc1 ; c1ccc(-c2nc(-c3ccccc3)c3cc(-c4cccc(-c5ccc6c(c5)oc5ccccc56)c4)ccc3n2)cc1]

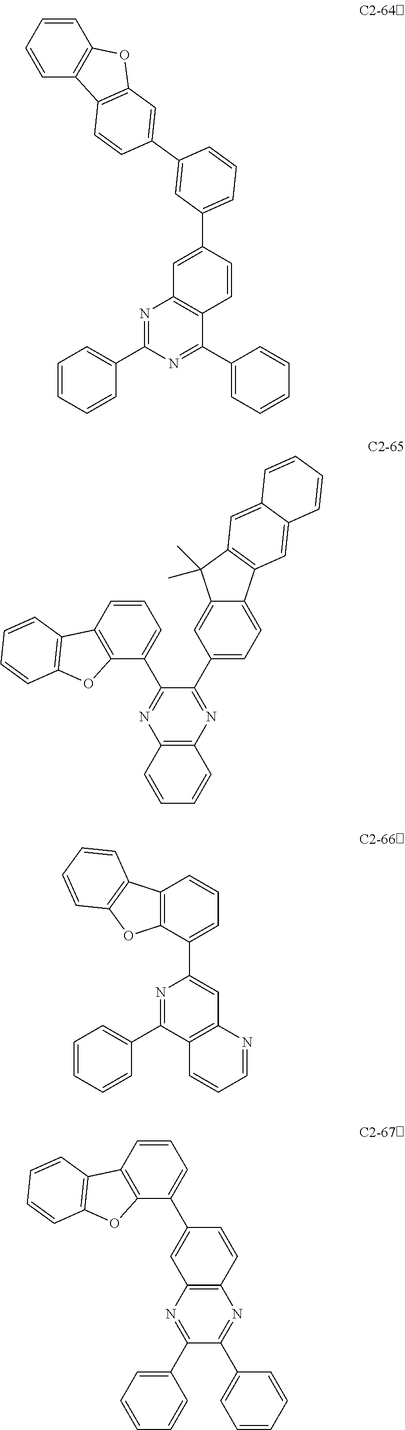 [SMILES: c1ccc(-c2nc(-c3ccccc3)c3ccc(-c4cccc(-c5ccc6c(c5)oc5ccccc56)c4)cc3n2)cc1 ; CC1(C)c2cc(-c3nc4ccccc4nc3-c3cccc4c3oc3ccccc34)ccc2-c2cc3ccccc3cc21 ; c1ccc(-c2nc(-c3cccc4c3oc3ccccc34)cc3ncccc23)cc1 ; c1ccc(-c2nc3ccc(-c4cccc5c4oc4ccccc45)cc3nc2-c2ccccc2)cc1]

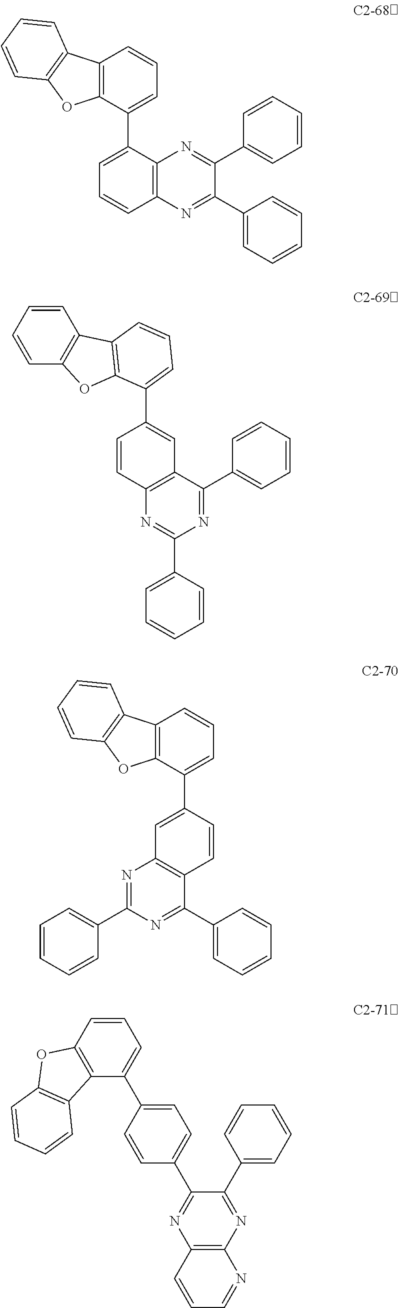 [SMILES: c1ccc(-c2nc3cccc(-c4cccc5c4oc4ccccc45)c3nc2-c2ccccc2)cc1 ; c1ccc(-c2nc(-c3ccccc3)c3cc(-c4cccc5c4oc4ccccc45)ccc3n2)cc1 ; c1ccc(-c2nc(-c3ccccc3)c3ccc(-c4cccc5c4oc4ccccc45)cc3n2)cc1 ; c1ccc(-c2nc3ncccc3nc2-c2ccc(-c3cccc4oc5ccccc5c34)cc2)cc1]

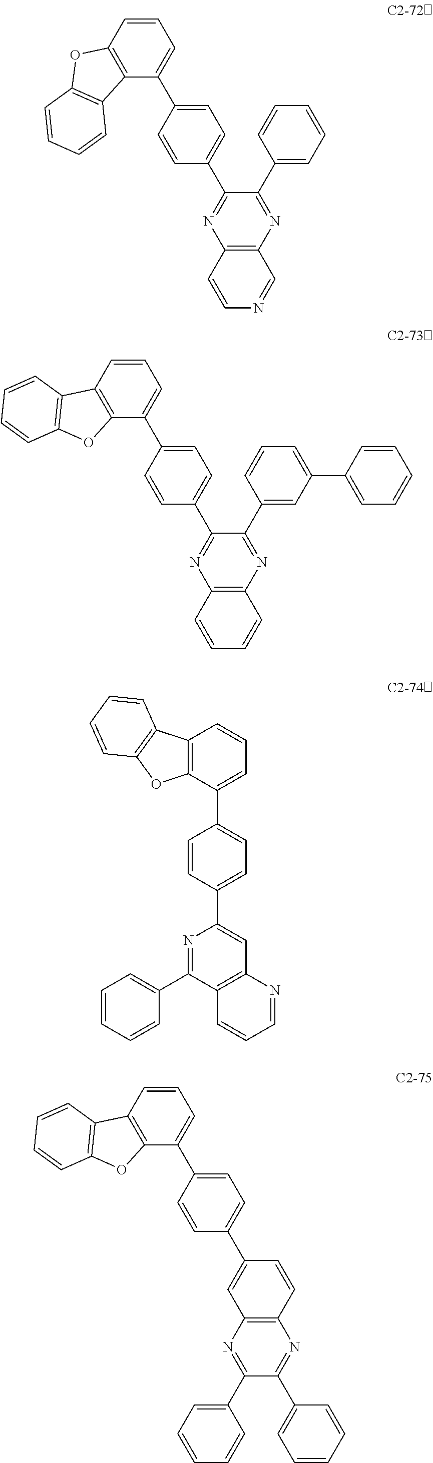 [SMILES: c1ccc(-c2nc3cnccc3nc2-c2ccc(-c3cccc4oc5ccccc5c34)cc2)cc1 ; c1ccc(-c2cccc(-c3nc4ccccc4nc3-c3ccc(-c4cccc5c4oc4ccccc45)cc3)c2)cc1 ; c1ccc(-c2nc(-c3ccc(-c4cccc5c4oc4ccccc45)cc3)cc3ncccc23)cc1 ; c1ccc(-c2nc3ccc(-c4ccc(-c5cccc6c5oc5ccccc56)cc4)cc3nc2-c2ccccc2)cc1]

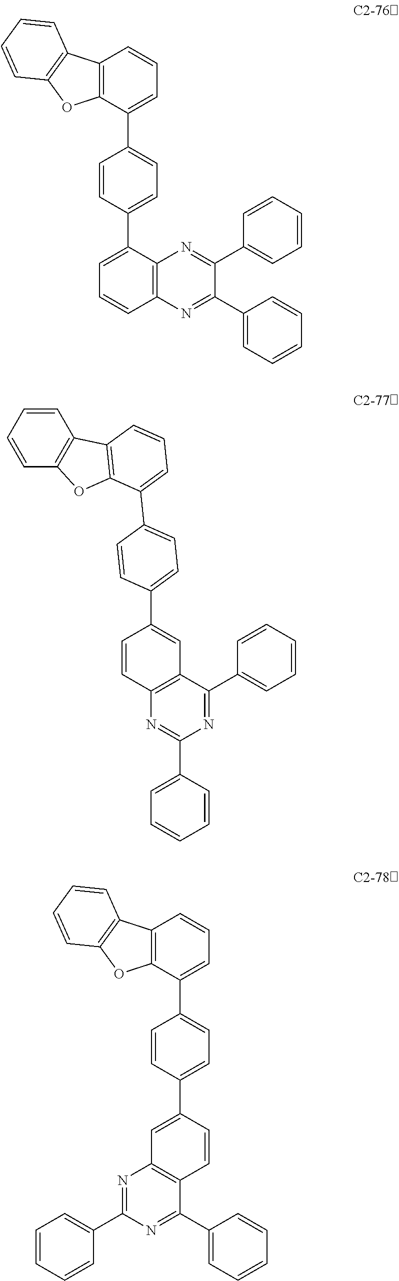 [SMILES: c1ccc(-c2nc3cccc(-c4ccc(-c5cccc6c5oc5ccccc56)cc4)c3nc2-c2ccccc2)cc1 ; c1ccc(-c2nc(-c3ccccc3)c3cc(-c4ccc(-c5cccc6c5oc5ccccc56)cc4)ccc3n2)cc1 ; c1ccc(-c2nc(-c3ccccc3)c3ccc(-c4ccc(-c5cccc6c5oc5ccccc56)cc4)cc3n2)cc1]

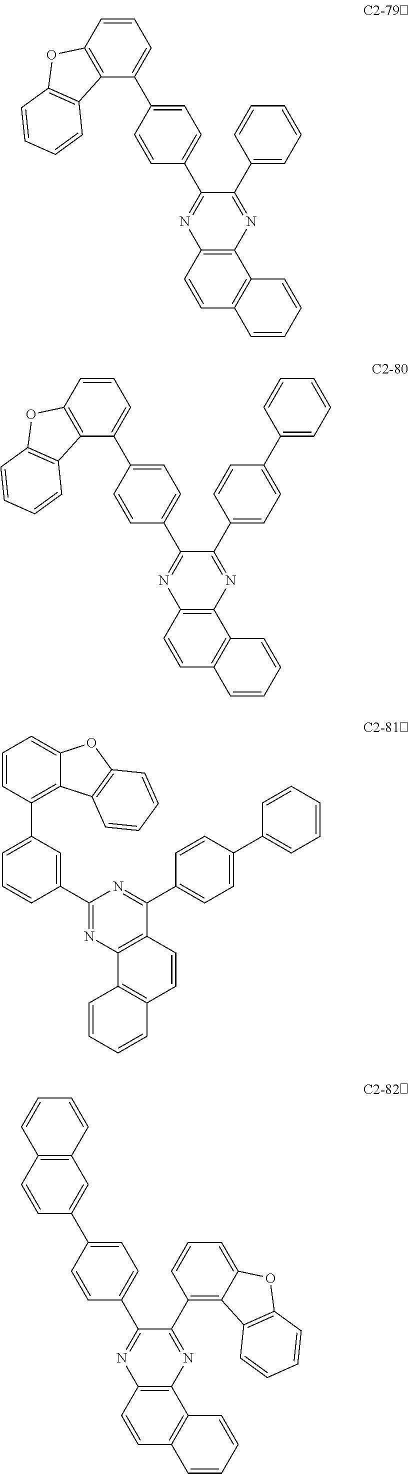

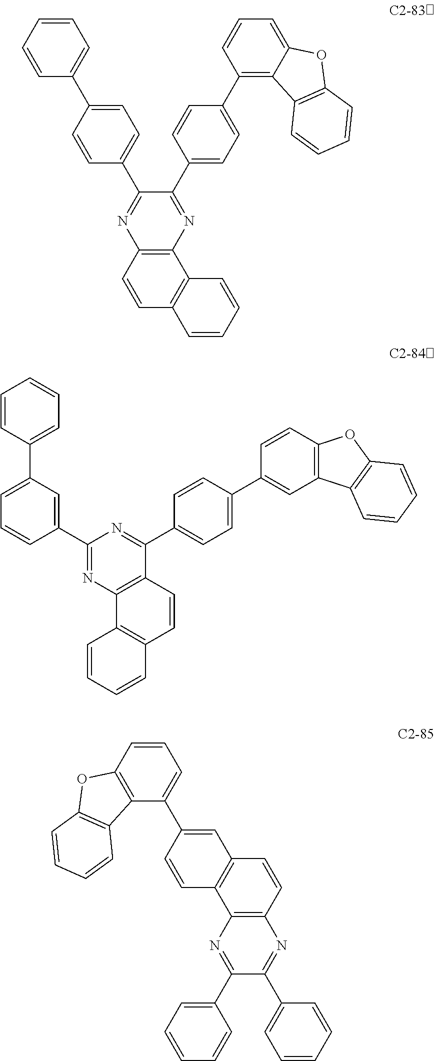 [SMILES: c1ccc(-c2ccc(-c3nc4ccc5ccccc5c4nc3-c3ccc(-c4cccc5oc6ccccc6c45)cc3)cc2)cc1 ; c1ccc(-c2cccc(-c3nc(-c4ccc(-c5ccc6oc7ccccc7c6c5)cc4)c4ccc5ccccc5c4n3)c2)cc1 ; c1ccc(-c2nc3ccc4cc(-c5cccc6oc7ccccc7c56)ccc4c3nc2-c2ccccc2)cc1]

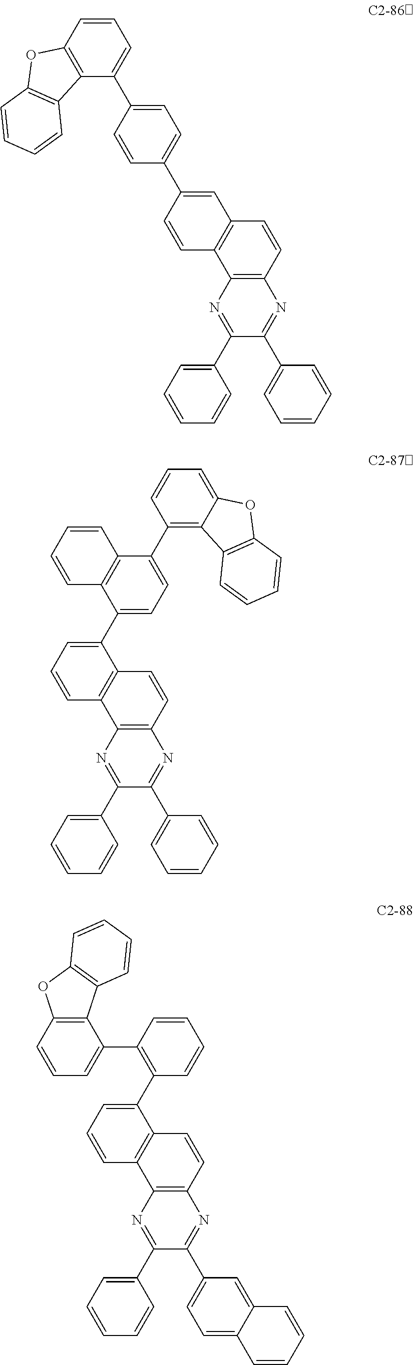 [SMILES: c1ccc(-c2nc3ccc4cc(-c5ccc(-c6cccc7oc8ccccc8c67)cc5)ccc4c3nc2-c2ccccc2)cc1 ; c1ccc(-c2nc3ccc4c(-c5ccc(-c6cccc7oc8ccccc8c67)c6ccccc56)cccc4c3nc2-c2ccccc2)cc1 ; c1ccc(-c2nc3c(ccc4c(-c5ccccc5-c5cccc6oc7ccccc7c56)cccc43)nc2-c2ccc3ccccc3c2)cc1]

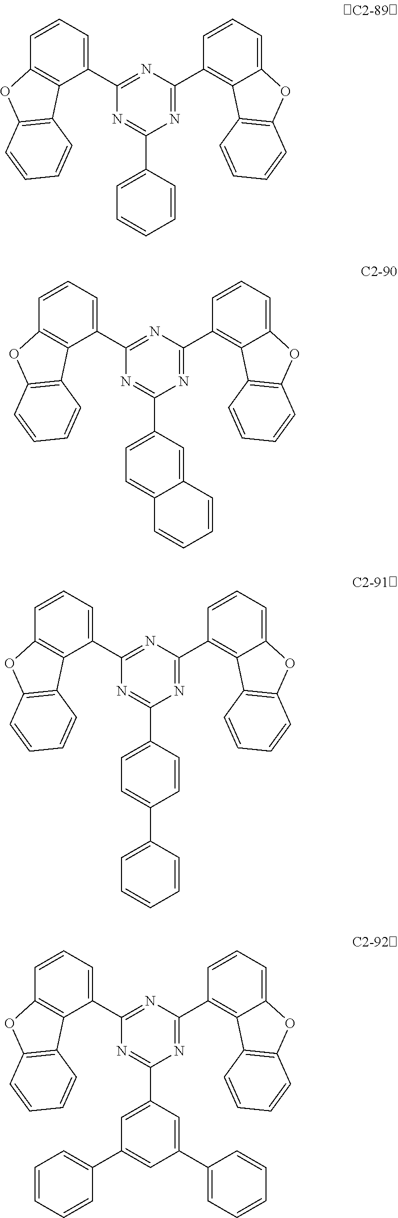 [SMILES: c1ccc(-c2nc(-c3cccc4oc5ccccc5c34)nc(-c3cccc4oc5ccccc5c34)n2)cc1 ; c1ccc2cc(-c3nc(-c4cccc5oc6ccccc6c45)nc(-c4cccc5oc6ccccc6c45)n3)ccc2c1 ; c1ccc(-c2ccc(-c3nc(-c4cccc5oc6ccccc6c45)nc(-c4cccc5oc6ccccc6c45)n3)cc2)cc1 ; c1ccc(-c2cc(-c3ccccc3)cc(-c3nc(-c4cccc5oc6ccccc6c45)nc(-c4cccc5oc6ccccc6c45)n3)c2)cc1]

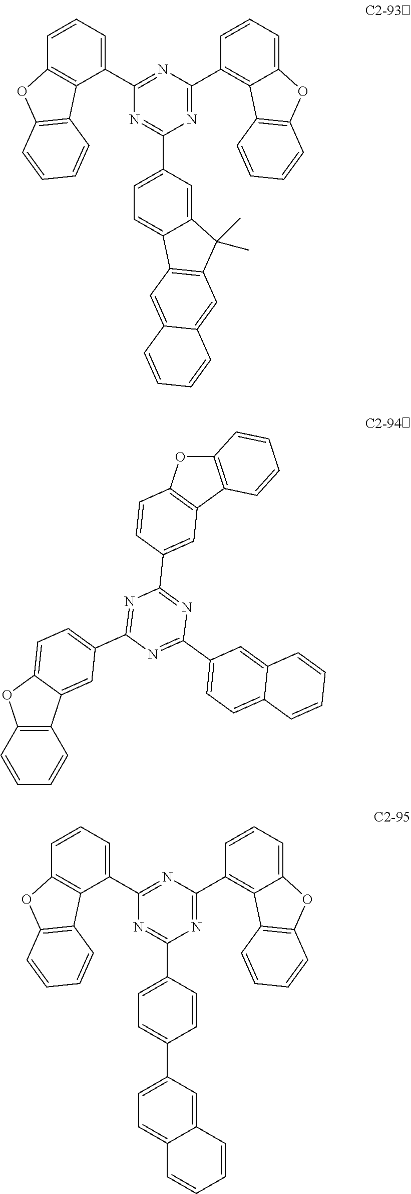 [SMILES: CC1(C)c2cc(-c3nc(-c4cccc5oc6ccccc6c45)nc(-c4cccc5oc6ccccc6c45)n3)ccc2-c2cc3ccccc3cc21 ; c1ccc2cc(-c3nc(-c4ccc5oc6ccccc6c5c4)nc(-c4ccc5oc6ccccc6c5c4)n3)ccc2c1 ; c1ccc2cc(-c3ccc(-c4nc(-c5cccc6oc7ccccc7c56)nc(-c5cccc6oc7ccccc7c56)n4)cc3)ccc2c1]

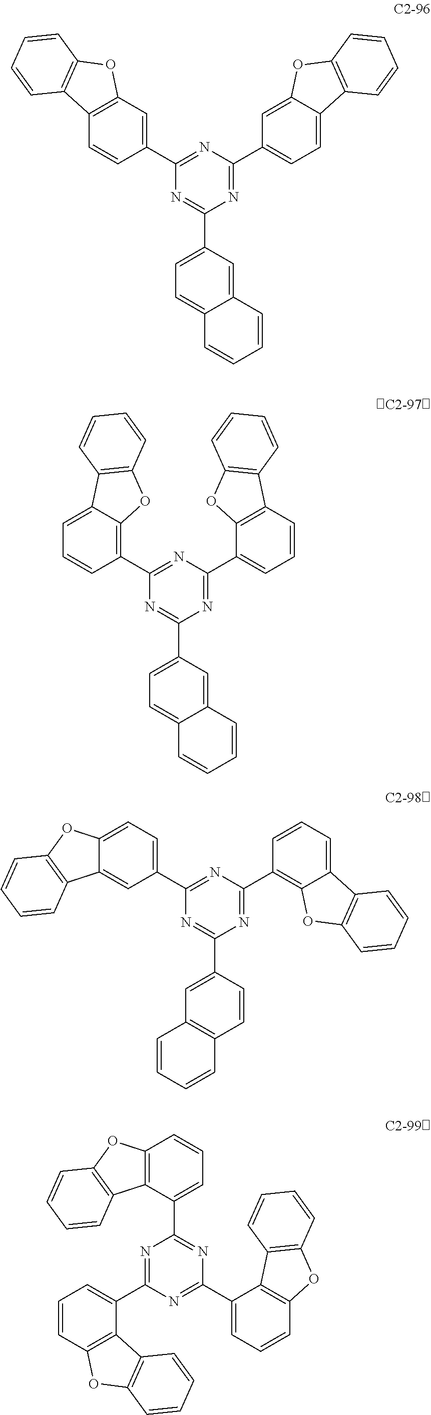 [SMILES: c1ccc2cc(-c3nc(-c4ccc5c(c4)oc4ccccc45)nc(-c4ccc5c(c4)oc4ccccc45)n3)ccc2c1 ; c1ccc2cc(-c3nc(-c4cccc5c4oc4ccccc45)nc(-c4cccc5c4oc4ccccc45)n3)ccc2c1 ; c1ccc2cc(-c3nc(-c4ccc5oc6ccccc6c5c4)nc(-c4cccc5c4oc4ccccc45)n3)ccc2c1 ; c1ccc2c(c1)oc1cccc(-c3nc(-c4cccc5oc6ccccc6c45)nc(-c4cccc5oc6ccccc6c45)n3)c12]

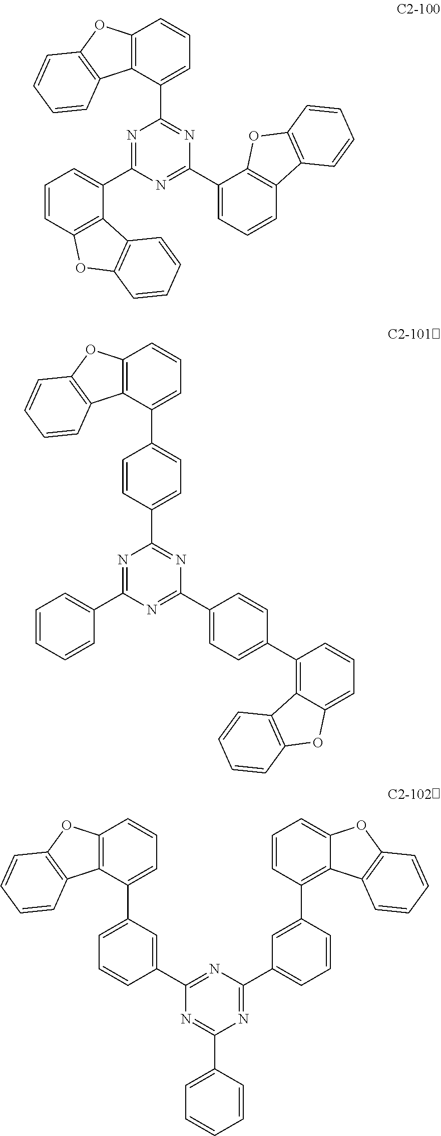 [SMILES: c1ccc2c(c1)oc1c(-c3nc(-c4cccc5oc6ccccc6c45)nc(-c4cccc5oc6ccccc6c45)n3)cccc12 ; c1ccc(-c2nc(-c3ccc(-c4cccc5oc6ccccc6c45)cc3)nc(-c3ccc(-c4cccc5oc6ccccc6c45)cc3)n2)cc1 ; c1ccc(-c2nc(-c3cccc(-c4cccc5oc6ccccc6c45)c3)nc(-c3cccc(-c4cccc5oc6ccccc6c45)c3)n2)cc1]

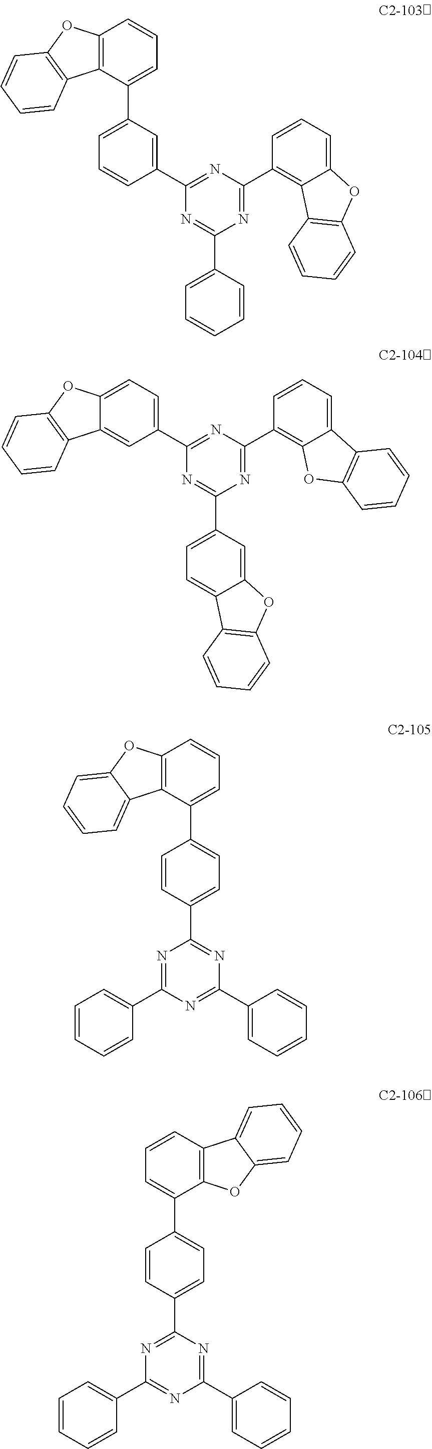 [SMILES: c1ccc(-c2nc(-c3cccc(-c4cccc5oc6ccccc6c45)c3)nc(-c3cccc4oc5ccccc5c34)n2)cc1 ; c1ccc2c(c1)oc1cc(-c3nc(-c4ccc5oc6ccccc6c5c4)nc(-c4cccc5c4oc4ccccc45)n3)ccc12 ; c1ccc(-c2nc(-c3ccccc3)nc(-c3ccc(-c4cccc5oc6ccccc6c45)cc3)n2)cc1 ; c1ccc(-c2nc(-c3ccccc3)nc(-c3ccc(-c4cccc5c4oc4ccccc45)cc3)n2)cc1]

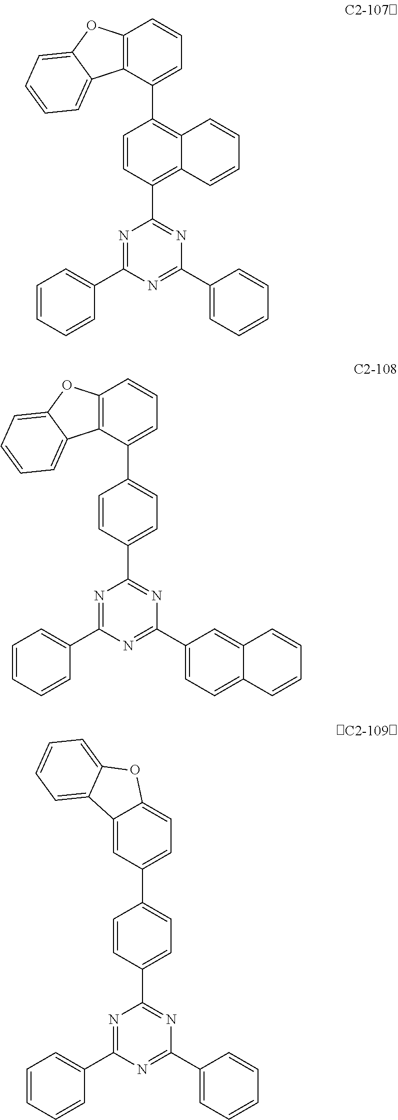 [SMILES: c1ccc(-c2nc(-c3ccccc3)nc(-c3ccc(-c4cccc5oc6ccccc6c45)c4ccccc34)n2)cc1 ; c1ccc(-c2nc(-c3ccc(-c4cccc5oc6ccccc6c45)cc3)nc(-c3ccc4ccccc4c3)n2)cc1 ; c1ccc(-c2nc(-c3ccccc3)nc(-c3ccc(-c4ccc5oc6ccccc6c5c4)cc3)n2)cc1]

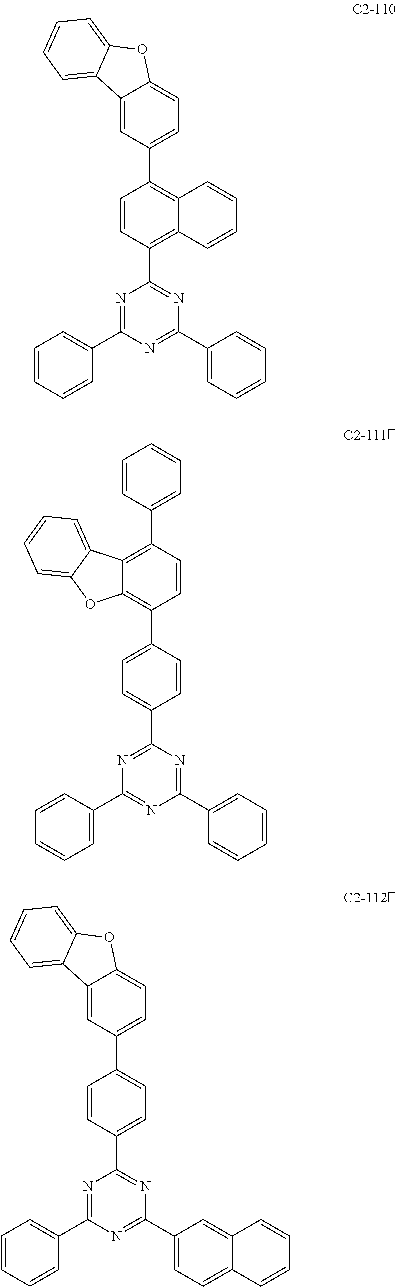 [SMILES: c1ccc(-c2nc(-c3ccccc3)nc(-c3ccc(-c4ccc5oc6ccccc6c5c4)c4ccccc34)n2)cc1 ; c1ccc(-c2nc(-c3ccccc3)nc(-c3ccc(-c4ccc(-c5ccccc5)c5c4oc4ccccc45)cc3)n2)cc1 ; c1ccc(-c2nc(-c3ccc(-c4ccc5oc6ccccc6c5c4)cc3)nc(-c3ccc4ccccc4c3)n2)cc1]

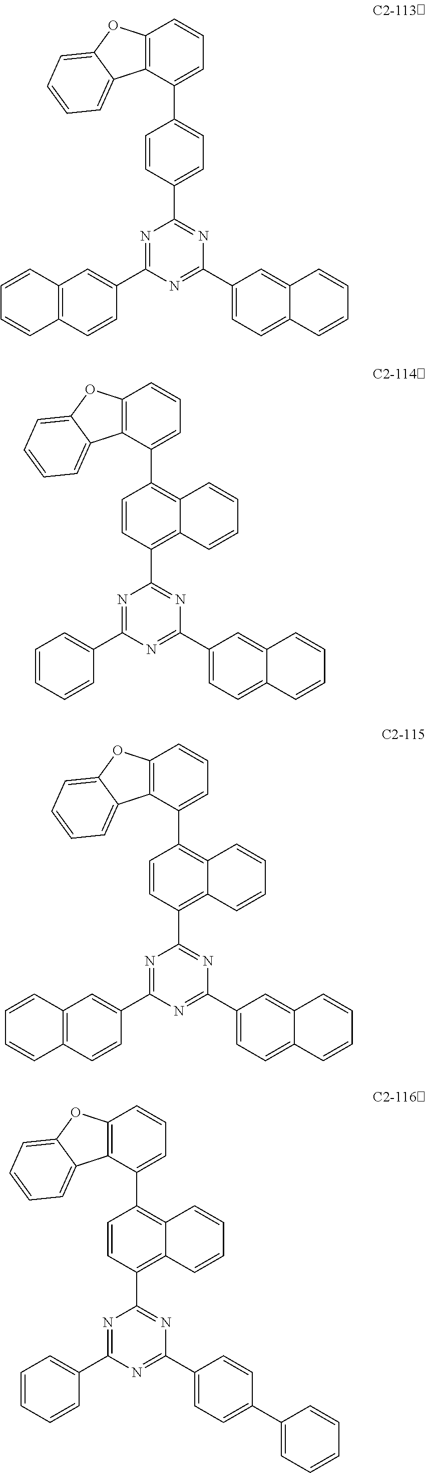 [SMILES: c1ccc2cc(-c3nc(-c4ccc(-c5cccc6oc7ccccc7c56)cc4)nc(-c4ccc5ccccc5c4)n3)ccc2c1 ; c1ccc(-c2nc(-c3ccc4ccccc4c3)nc(-c3ccc(-c4cccc5oc6ccccc6c45)c4ccccc34)n2)cc1 ; c1ccc2cc(-c3nc(-c4ccc5ccccc5c4)nc(-c4ccc(-c5cccc6oc7ccccc7c56)c5ccccc45)n3)ccc2c1 ; c1ccc(-c2ccc(-c3nc(-c4ccccc4)nc(-c4ccc(-c5cccc6oc7ccccc7c56)c5ccccc45)n3)cc2)cc1]

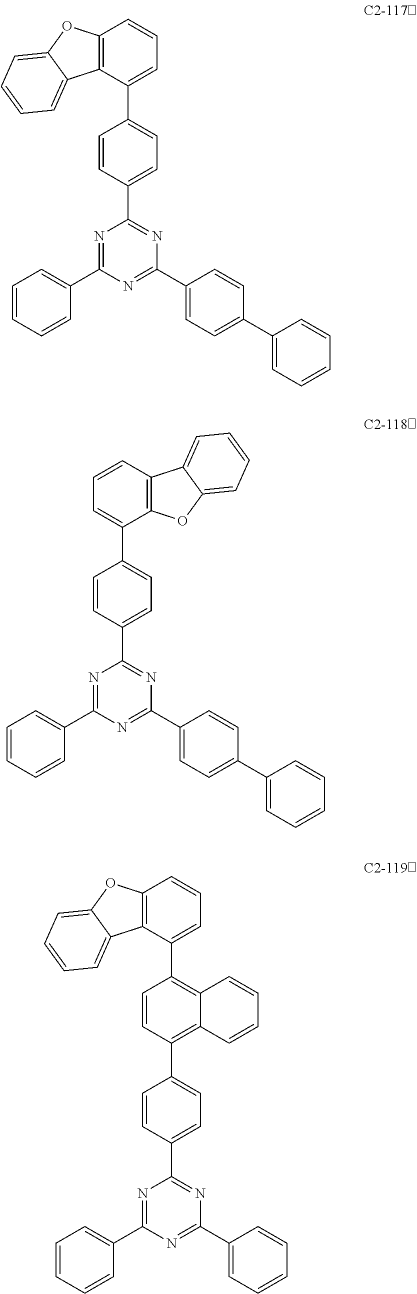 [SMILES: c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccccc4)nc(-c4ccc(-c5cccc6c5oc5ccccc56)cc4)n3)cc2)cc1 ; c1ccc(-c2nc(-c3ccccc3)nc(-c3ccc(-c4ccc(-c5cccc6oc7ccccc7c56)c5ccccc45)cc3)n2)cc1]

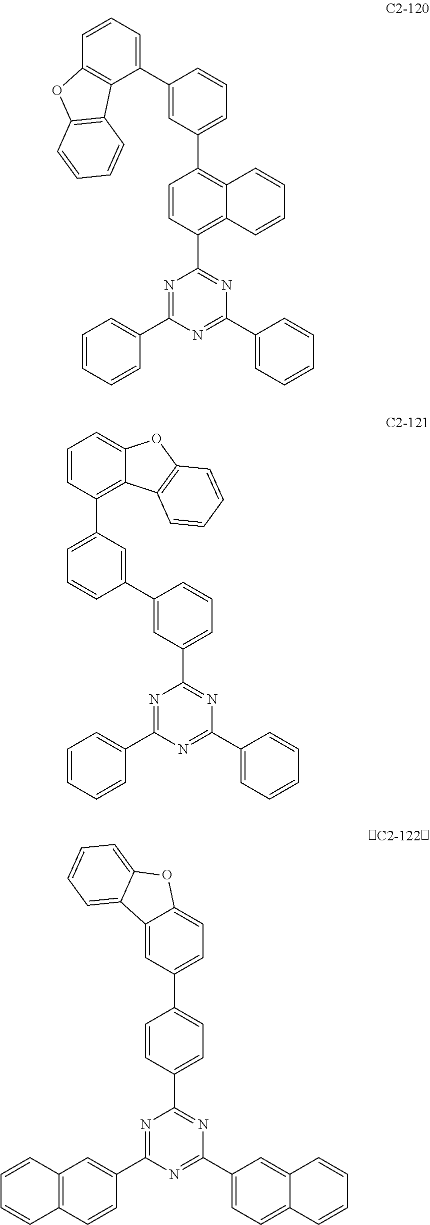 [SMILES: c1ccc(-c2nc(-c3ccccc3)nc(-c3ccc(-c4cccc(-c5cccc6oc7ccccc7c56)c4)c4ccccc34)n2)cc1 ; c1ccc(-c2nc(-c3ccccc3)nc(-c3cccc(-c4cccc(-c5cccc6oc7ccccc7c56)c4)c3)n2)cc1 ; c1ccc2cc(-c3nc(-c4ccc(-c5ccc6oc7ccccc7c6c5)cc4)nc(-c4ccc5ccccc5c4)n3)ccc2c1]

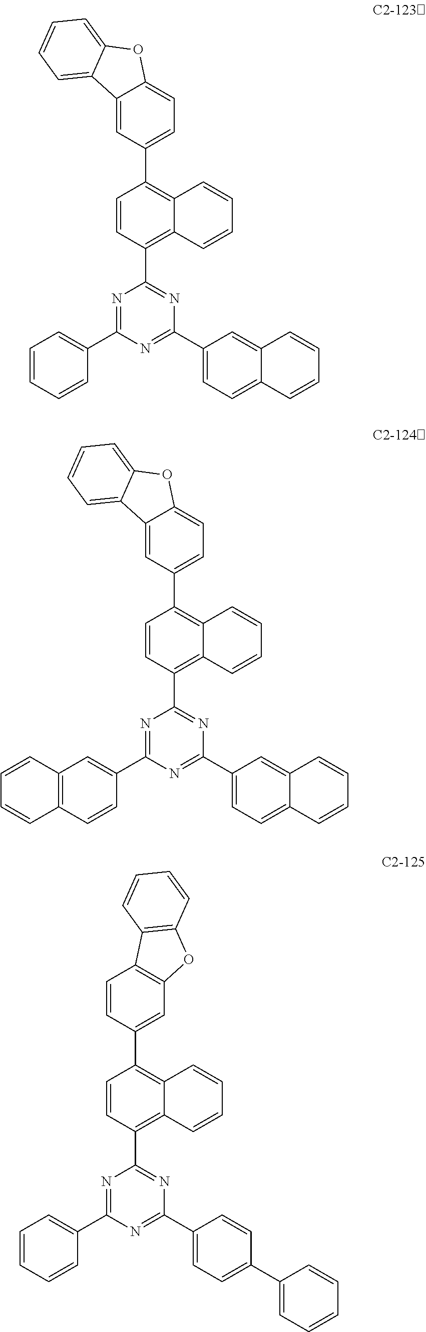 [SMILES: c1ccc(-c2nc(-c3ccc4ccccc4c3)nc(-c3ccc(-c4ccc5oc6ccccc6c5c4)c4ccccc34)n2)cc1 ; c1ccc2cc(-c3nc(-c4ccc5ccccc5c4)nc(-c4ccc(-c5ccc6oc7ccccc7c6c5)c5ccccc45)n3)ccc2c1 ; c1ccc(-c2ccc(-c3nc(-c4ccccc4)nc(-c4ccc(-c5ccc6c(c5)oc5ccccc56)c5ccccc45)n3)cc2)cc1]

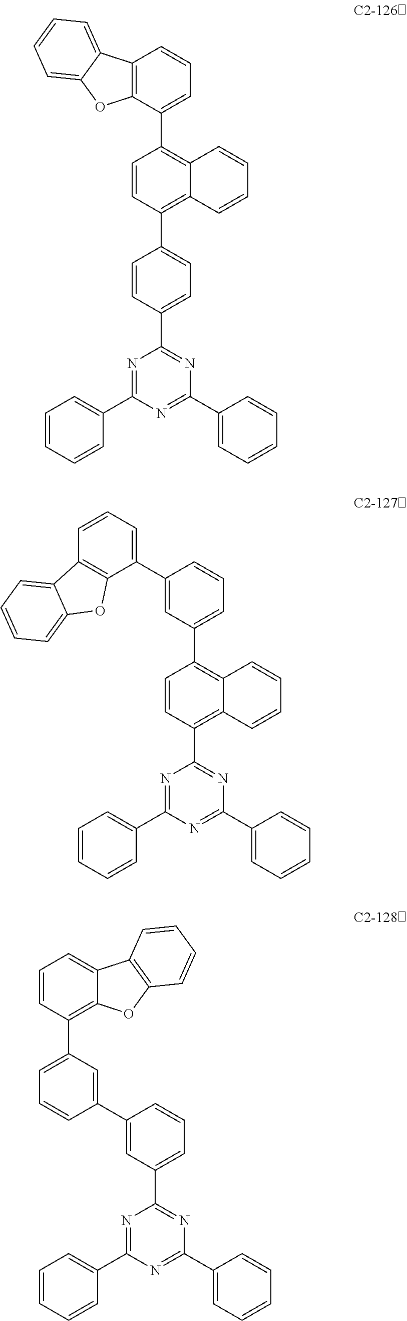 [SMILES: c1ccc(-c2nc(-c3ccccc3)nc(-c3ccc(-c4ccc(-c5cccc6c5oc5ccccc56)c5ccccc45)cc3)n2)cc1 ; c1ccc(-c2nc(-c3ccccc3)nc(-c3ccc(-c4cccc(-c5cccc6c5oc5ccccc56)c4)c4ccccc34)n2)cc1 ; c1ccc(-c2nc(-c3ccccc3)nc(-c3cccc(-c4cccc(-c5cccc6c5oc5ccccc56)c4)c3)n2)cc1]

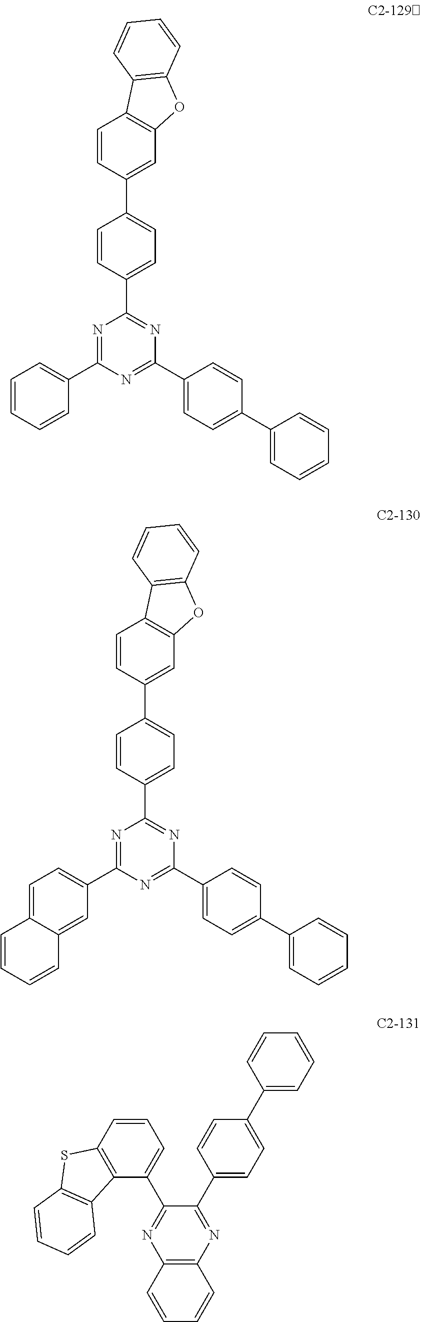 [SMILES: c1ccc(-c2ccc(-c3nc(-c4ccccc4)nc(-c4ccc(-c5ccc6c(c5)oc5ccccc56)cc4)n3)cc2)cc1 ; c1ccc(-c2ccc(-c3nc(-c4ccc(-c5ccc6c(c5)oc5ccccc56)cc4)nc(-c4ccc5ccccc5c4)n3)cc2)cc1 ; c1ccc(-c2ccc(-c3nc4ccccc4nc3-c3cccc4sc5ccccc5c34)cc2)cc1]

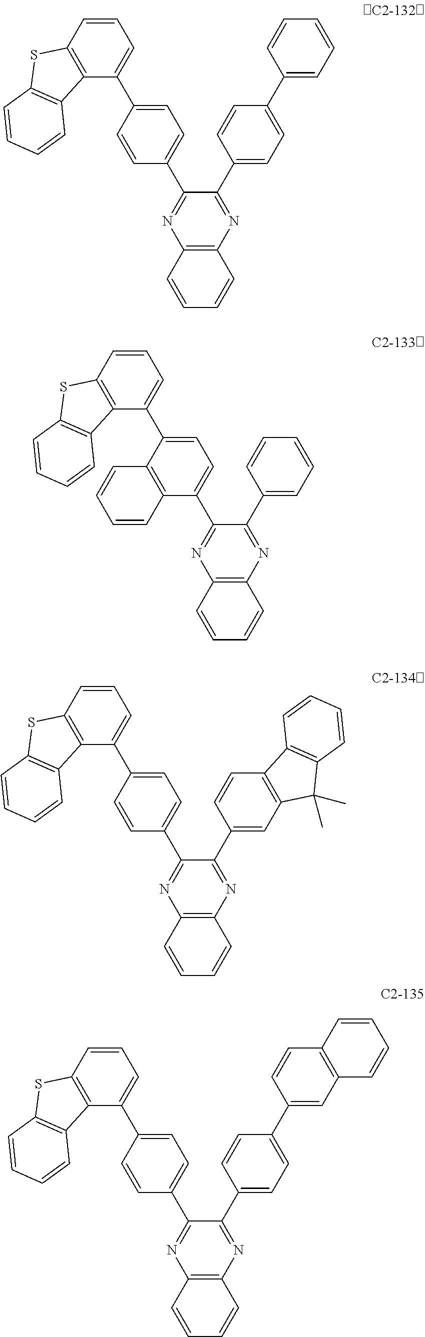 [SMILES: c1ccc(-c2ccc(-c3nc4ccccc4nc3-c3ccc(-c4cccc5sc6ccccc6c45)cc3)cc2)cc1 ; c1ccc(-c2nc3ccccc3nc2-c2ccc(-c3cccc4sc5ccccc5c34)c3ccccc23)cc1 ; CC1(C)c2ccccc2-c2ccc(-c3nc4ccccc4nc3-c3ccc(-c4cccc5sc6ccccc6c45)cc3)cc21 ; c1ccc2cc(-c3ccc(-c4nc5ccccc5nc4-c4ccc(-c5cccc6sc7ccccc7c56)cc4)cc3)ccc2c1]

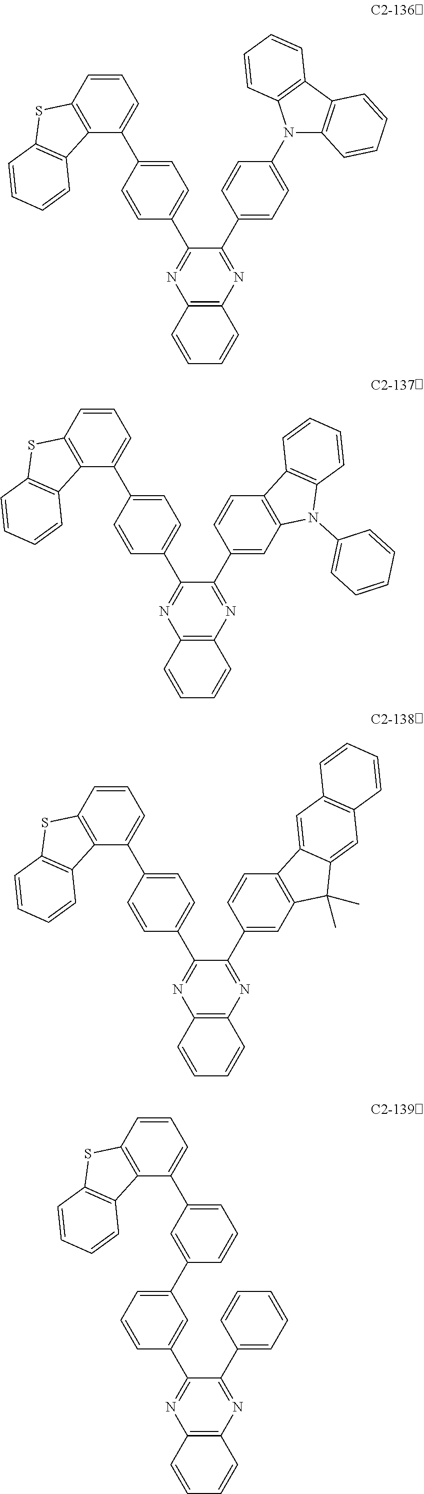 [SMILES: c1ccc2nc(-c3ccc(-n4c5ccccc5c5ccccc54)cc3)c(-c3ccc(-c4cccc5sc6ccccc6c45)cc3)nc2c1 ; c1ccc(-n2c3ccccc3c3ccc(-c4nc5ccccc5nc4-c4ccc(-c5cccc6sc7ccccc7c56)cc4)cc32)cc1 ; CC1(C)c2cc(-c3nc4ccccc4nc3-c3ccc(-c4cccc5sc6ccccc6c45)cc3)ccc2-c2cc3ccccc3cc21 ; c1ccc(-c2nc3ccccc3nc2-c2cccc(-c3cccc(-c4cccc5sc6ccccc6c45)c3)c2)cc1]

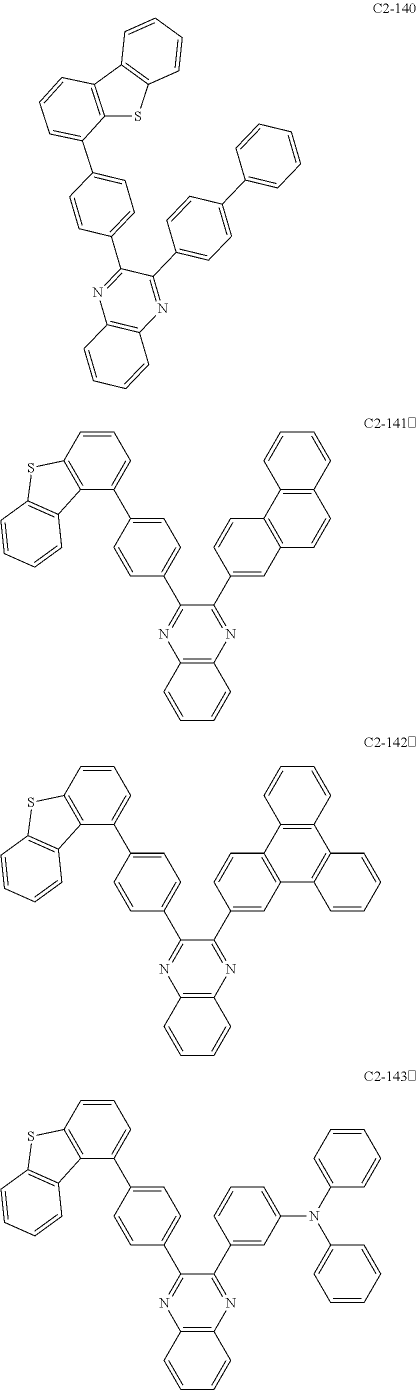

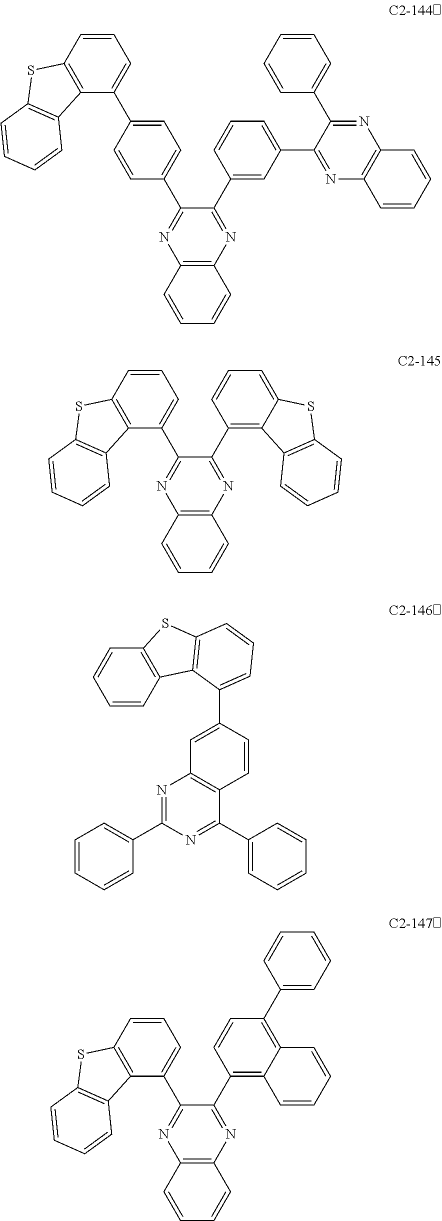

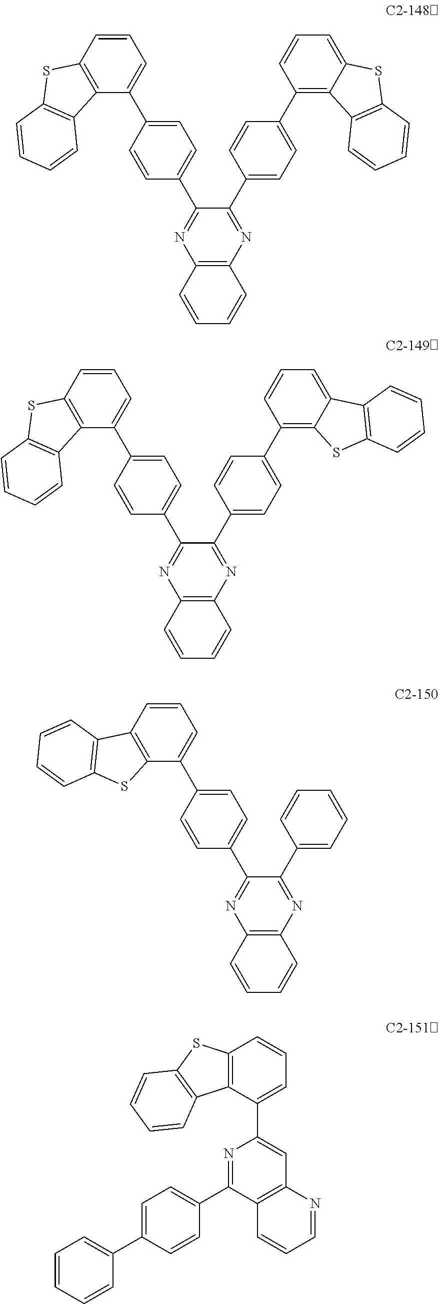

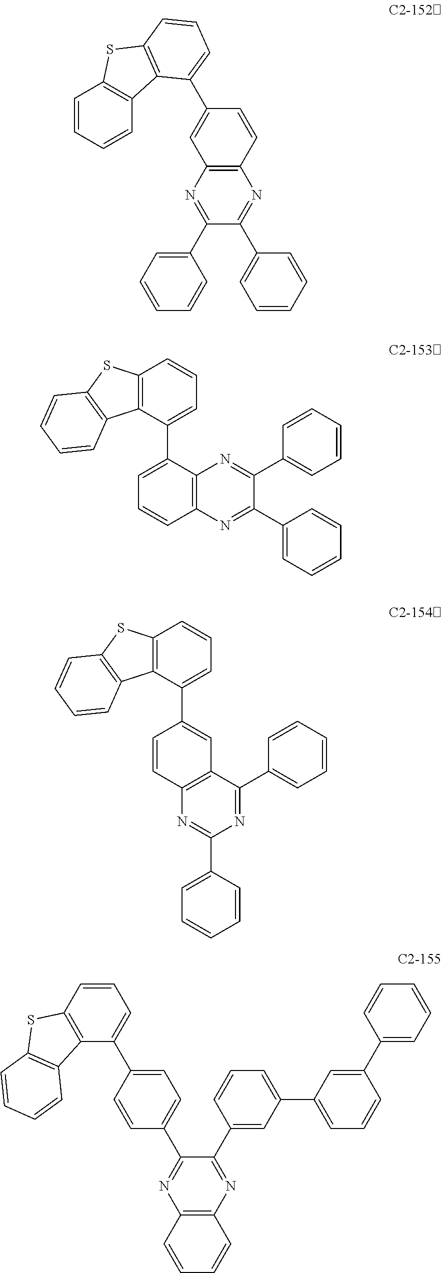 [SMILES: c1ccc(-c2nc3ccc(-c4cccc5sc6ccccc6c45)cc3nc2-c2ccccc2)cc1 ; c1ccc(-c2nc3cccc(-c4cccc5sc6ccccc6c45)c3nc2-c2ccccc2)cc1 ; c1ccc(-c2nc(-c3ccccc3)c3cc(-c4cccc5sc6ccccc6c45)ccc3n2)cc1 ; c1ccc(-c2cccc(-c3cccc(-c4nc5ccccc5nc4-c4ccc(-c5cccc6sc7ccccc7c56)cc4)c3)c2)cc1]

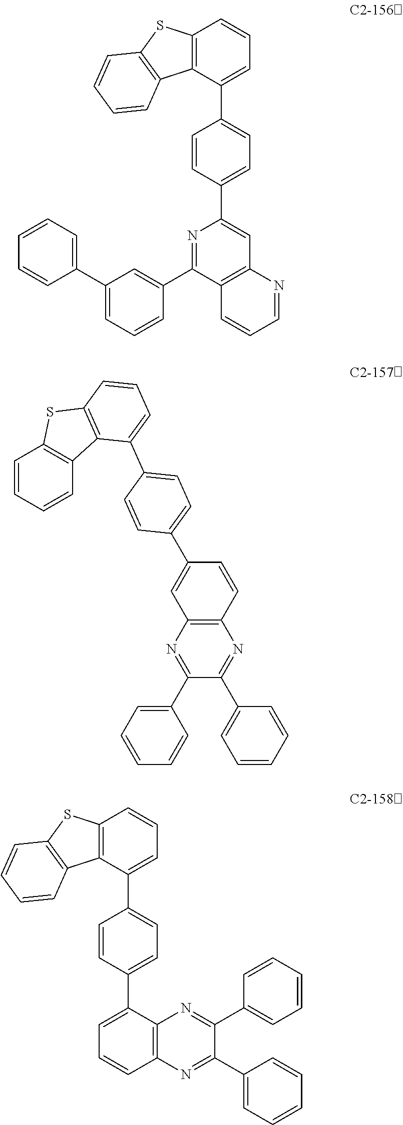

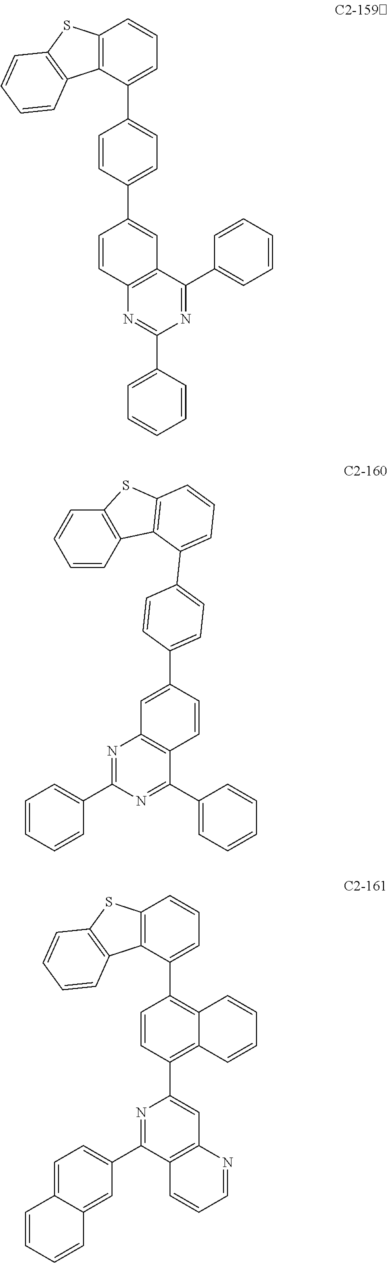 [SMILES: c1ccc(-c2nc(-c3ccccc3)c3cc(-c4ccc(-c5cccc6sc7ccccc7c56)cc4)ccc3n2)cc1 ; c1ccc(-c2nc(-c3ccccc3)c3ccc(-c4ccc(-c5cccc6sc7ccccc7c56)cc4)cc3n2)cc1 ; c1ccc2cc(-c3nc(-c4ccc(-c5cccc6sc7ccccc7c56)c5ccccc45)cc4ncccc34)ccc2c1]

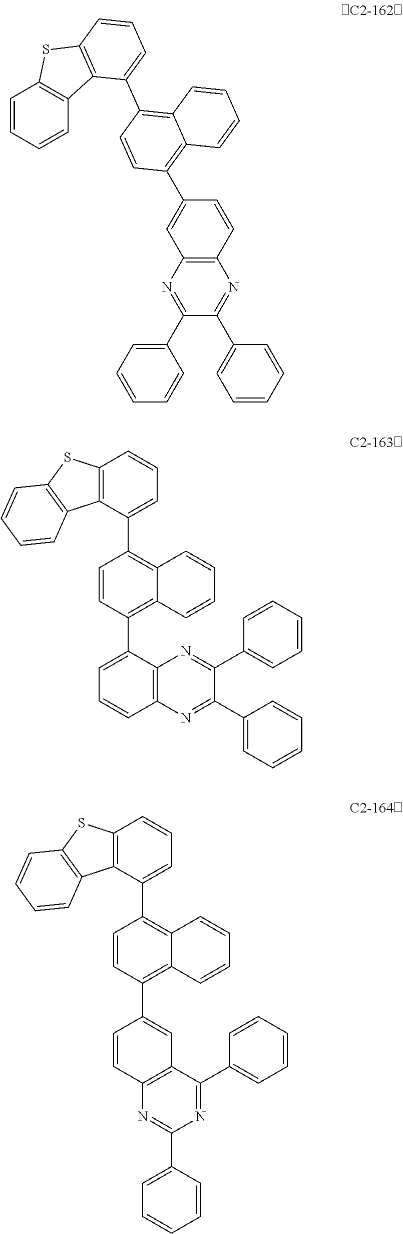 [SMILES: c1ccc(-c2nc3ccc(-c4ccc(-c5cccc6sc7ccccc7c56)c5ccccc45)cc3nc2-c2ccccc2)cc1 ; c1ccc(-c2nc3cccc(-c4ccc(-c5cccc6sc7ccccc7c56)c5ccccc45)c3nc2-c2ccccc2)cc1 ; c1ccc(-c2nc(-c3ccccc3)c3cc(-c4ccc(-c5cccc6sc7ccccc7c56)c5ccccc45)ccc3n2)cc1]

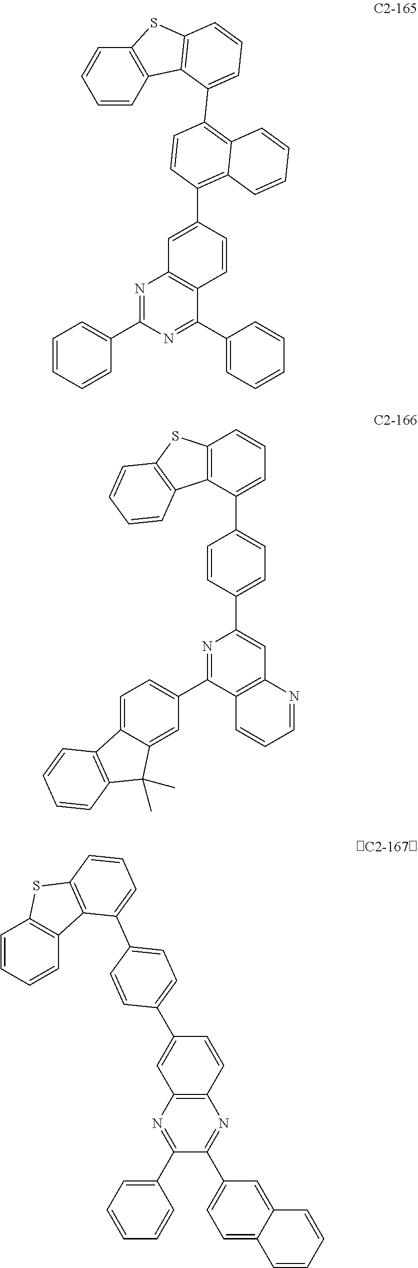

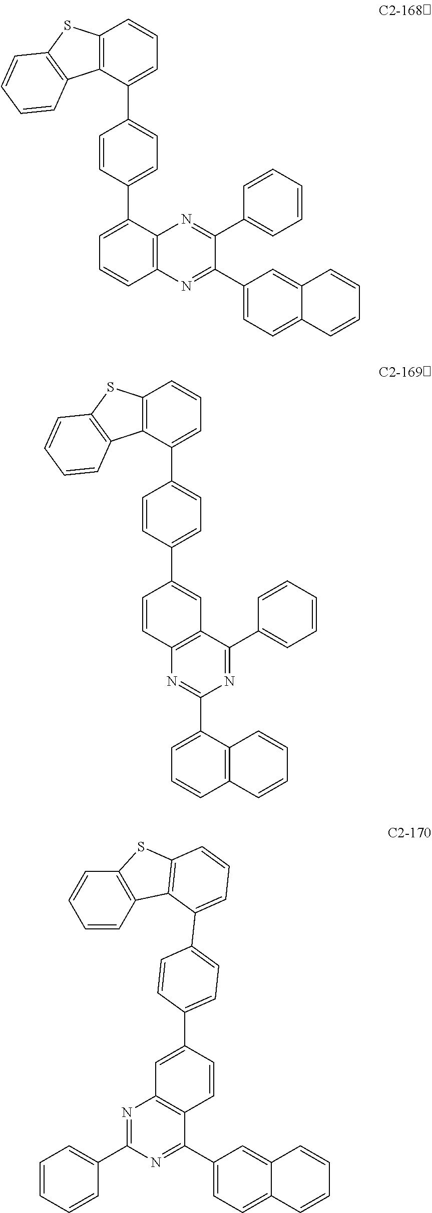 [SMILES: c1ccc(-c2nc3c(-c4ccc(-c5cccc6sc7ccccc7c56)cc4)cccc3nc2-c2ccc3ccccc3c2)cc1 ; c1ccc(-c2nc(-c3cccc4ccccc34)nc3ccc(-c4ccc(-c5cccc6sc7ccccc7c56)cc4)cc23)cc1 ; c1ccc(-c2nc(-c3ccc4ccccc4c3)c3ccc(-c4ccc(-c5cccc6sc7ccccc7c56)cc4)cc3n2)cc1]

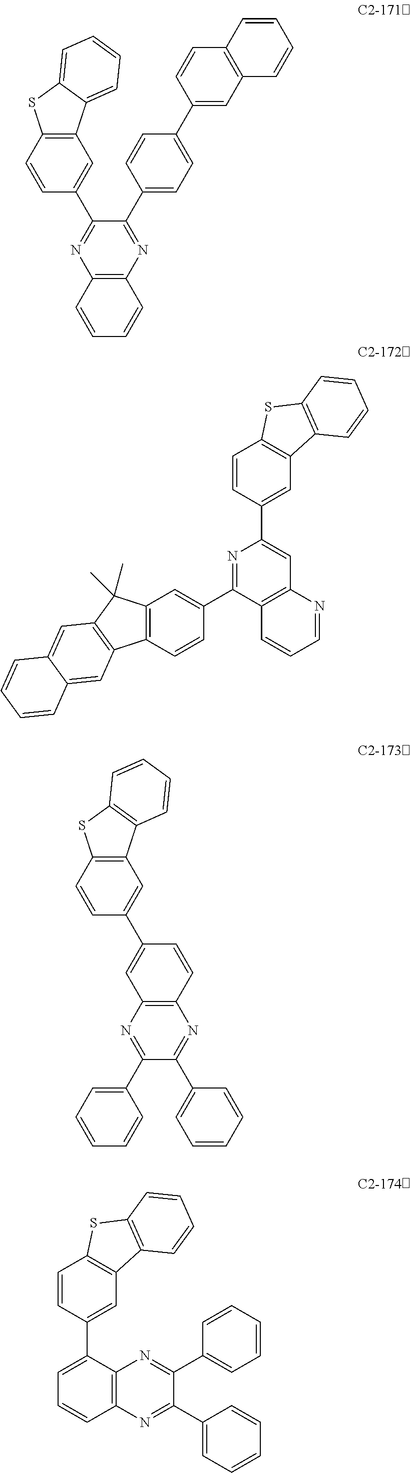 [SMILES: c1ccc2cc(-c3ccc(-c4nc5ccccc5nc4-c4ccc5sc6ccccc6c5c4)cc3)ccc2c1 ; CC1(C)c2cc(-c3nc(-c4ccc5sc6ccccc6c5c4)cc4ncccc34)ccc2-c2cc3ccccc3cc21 ; c1ccc(-c2nc3ccc(-c4ccc5sc6ccccc6c5c4)cc3nc2-c2ccccc2)cc1 ; c1ccc(-c2nc3cccc(-c4ccc5sc6ccccc6c5c4)c3nc2-c2ccccc2)cc1]

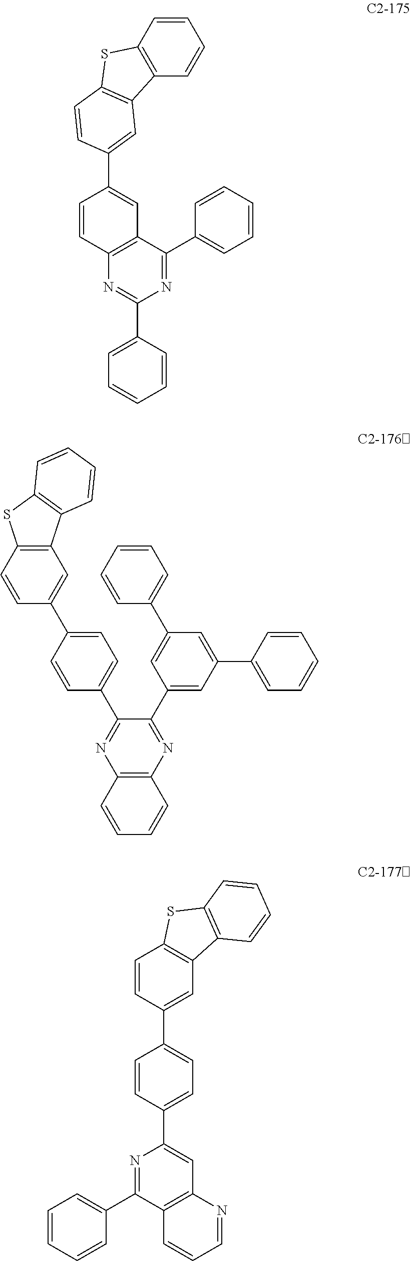 [SMILES: c1ccc(-c2nc(-c3ccccc3)c3cc(-c4ccc5sc6ccccc6c5c4)ccc3n2)cc1 ; c1ccc(-c2cc(-c3ccccc3)cc(-c3nc4ccccc4nc3-c3ccc(-c4ccc5sc6ccccc6c5c4)cc3)c2)cc1 ; c1ccc(-c2nc(-c3ccc(-c4ccc5sc6ccccc6c5c4)cc3)cc3ncccc23)cc1]

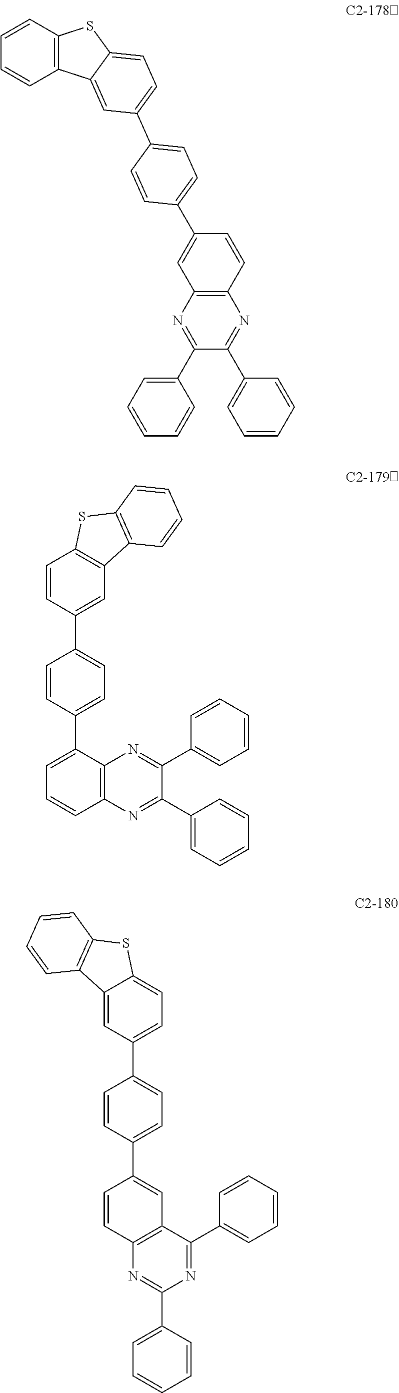 [SMILES: c1ccc(-c2nc3ccc(-c4ccc(-c5ccc6sc7ccccc7c6c5)cc4)cc3nc2-c2ccccc2)cc1 ; c1ccc(-c2nc3cccc(-c4ccc(-c5ccc6sc7ccccc7c6c5)cc4)c3nc2-c2ccccc2)cc1 ; c1ccc(-c2nc(-c3ccccc3)c3cc(-c4ccc(-c5ccc6sc7ccccc7c6c5)cc4)ccc3n2)cc1]

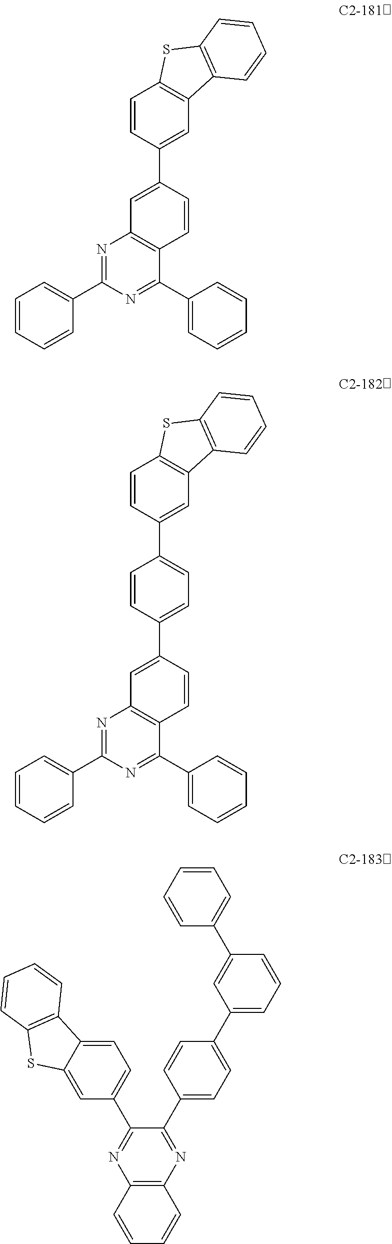

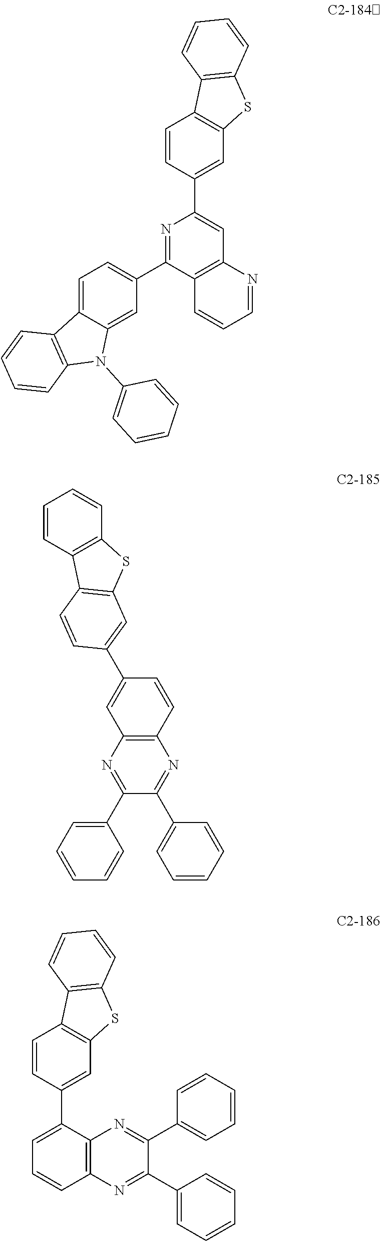 [SMILES: c1ccc(-n2c3ccccc3c3ccc(-c4nc(-c5ccc6c(c5)sc5ccccc56)cc5ncccc45)cc32)cc1 ; c1ccc(-c2nc3ccc(-c4ccc5c(c4)sc4ccccc45)cc3nc2-c2ccccc2)cc1 ; c1ccc(-c2nc3cccc(-c4ccc5c(c4)sc4ccccc45)c3nc2-c2ccccc2)cc1]

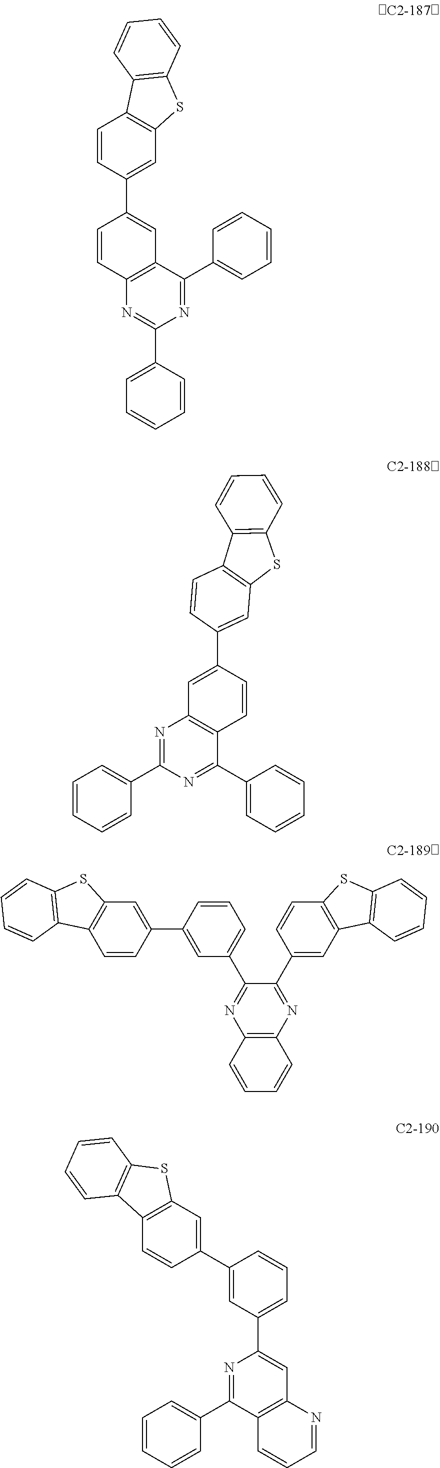 [SMILES: c1ccc(-c2nc(-c3ccccc3)c3cc(-c4ccc5c(c4)sc4ccccc45)ccc3n2)cc1 ; c1ccc(-c2nc(-c3ccccc3)c3ccc(-c4ccc5c(c4)sc4ccccc45)cc3n2)cc1 ; c1cc(-c2ccc3c(c2)sc2ccccc23)cc(-c2nc3ccccc3nc2-c2ccc3sc4ccccc4c3c2)c1 ; c1ccc(-c2nc(-c3cccc(-c4ccc5c(c4)sc4ccccc45)c3)cc3ncccc23)cc1]

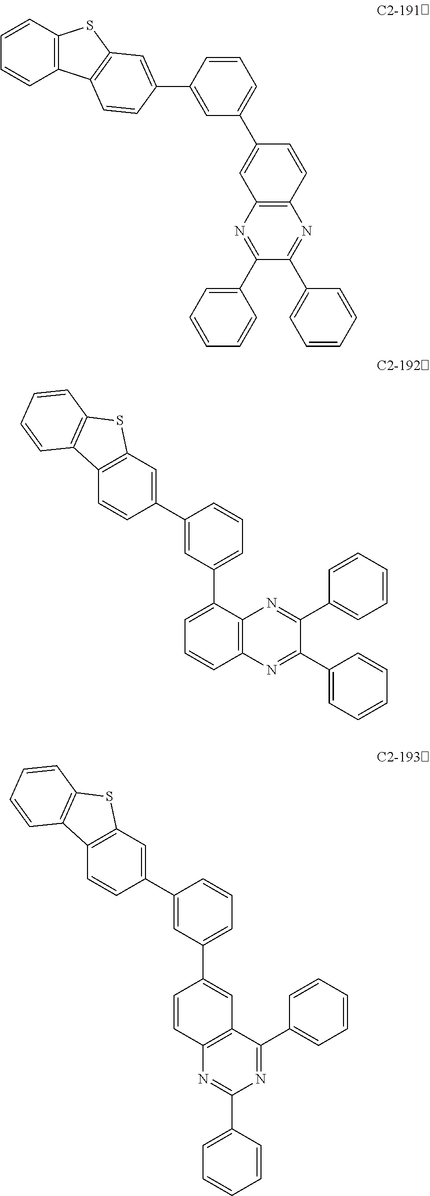 [SMILES: c1ccc(-c2nc3ccc(-c4cccc(-c5ccc6c(c5)sc5ccccc56)c4)cc3nc2-c2ccccc2)cc1 ; c1ccc(-c2nc3cccc(-c4cccc(-c5ccc6c(c5)sc5ccccc56)c4)c3nc2-c2ccccc2)cc1 ; c1ccc(-c2nc(-c3ccccc3)c3cc(-c4cccc(-c5ccc6c(c5)sc5ccccc56)c4)ccc3n2)cc1]

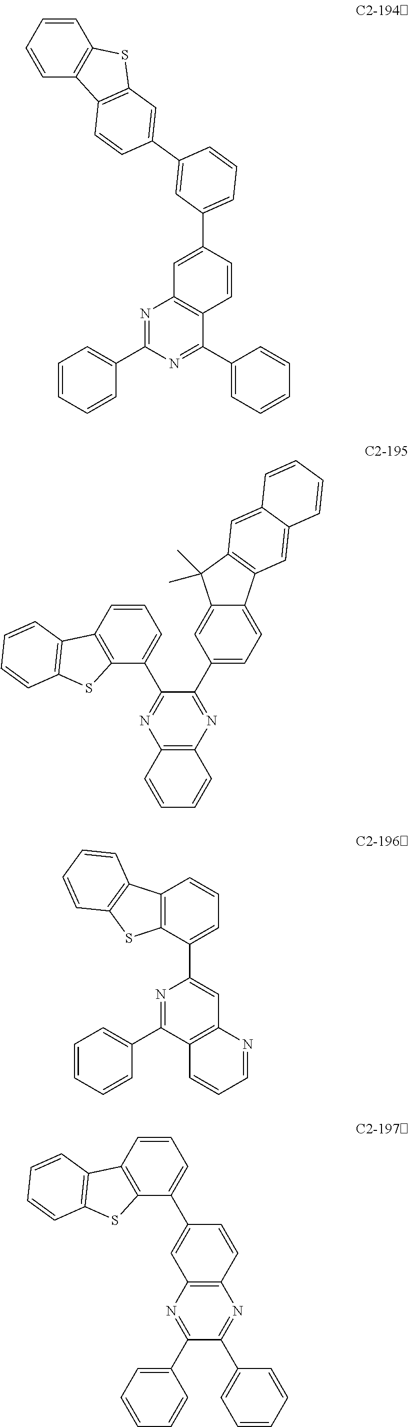 [SMILES: c1ccc(-c2nc(-c3ccccc3)c3ccc(-c4cccc(-c5ccc6c(c5)sc5ccccc56)c4)cc3n2)cc1 ; CC1(C)c2cc(-c3nc4ccccc4nc3-c3cccc4c3sc3ccccc34)ccc2-c2cc3ccccc3cc21 ; c1ccc(-c2nc(-c3cccc4c3sc3ccccc34)cc3ncccc23)cc1 ; c1ccc(-c2nc3ccc(-c4cccc5c4sc4ccccc45)cc3nc2-c2ccccc2)cc1]

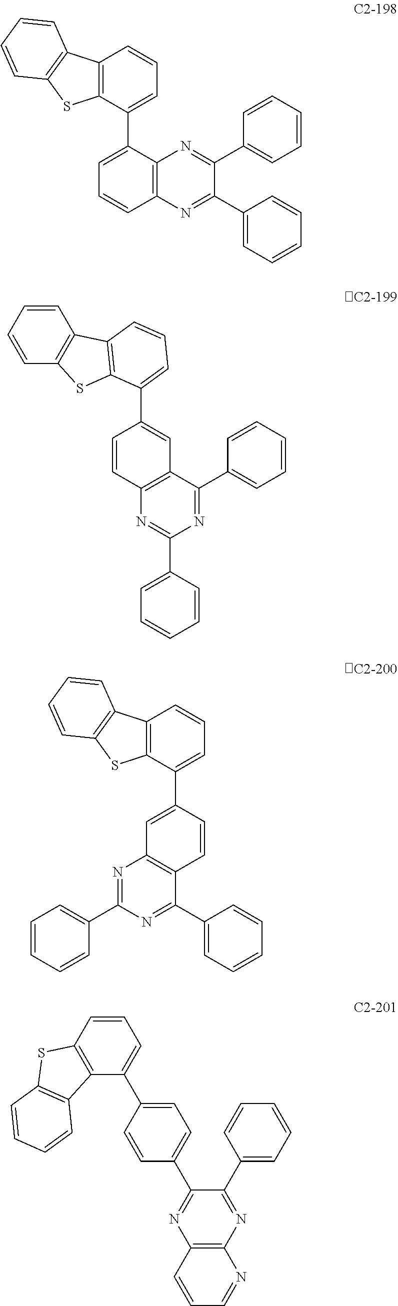 [SMILES: c1ccc(-c2nc3cccc(-c4cccc5c4sc4ccccc45)c3nc2-c2ccccc2)cc1 ; c1ccc(-c2nc(-c3ccccc3)c3cc(-c4cccc5c4sc4ccccc45)ccc3n2)cc1 ; c1ccc(-c2nc(-c3ccccc3)c3ccc(-c4cccc5c4sc4ccccc45)cc3n2)cc1 ; c1ccc(-c2nc3ncccc3nc2-c2ccc(-c3cccc4sc5ccccc5c34)cc2)cc1]

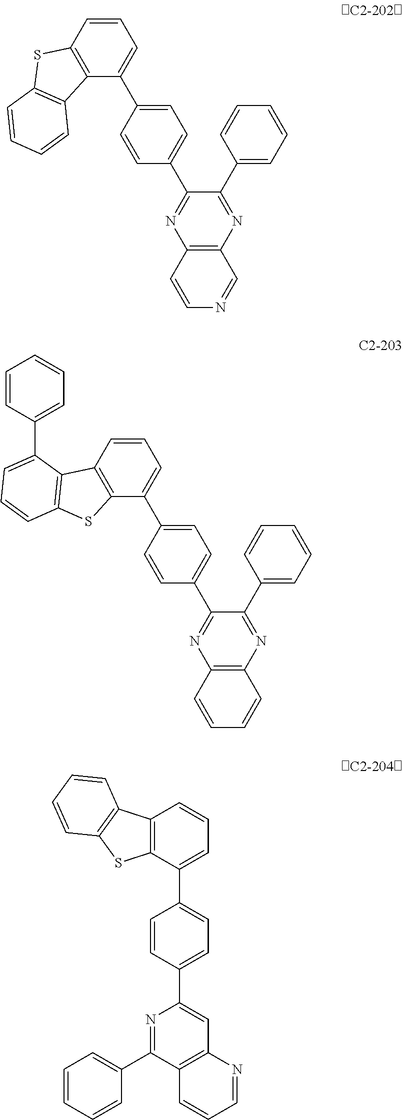 [SMILES: c1ccc(-c2nc3cnccc3nc2-c2ccc(-c3cccc4sc5ccccc5c34)cc2)cc1 ; c1ccc(-c2nc3ccccc3nc2-c2ccc(-c3cccc4c3sc3cccc(-c5ccccc5)c34)cc2)cc1 ; c1ccc(-c2nc(-c3ccc(-c4cccc5c4sc4ccccc45)cc3)cc3ncccc23)cc1]

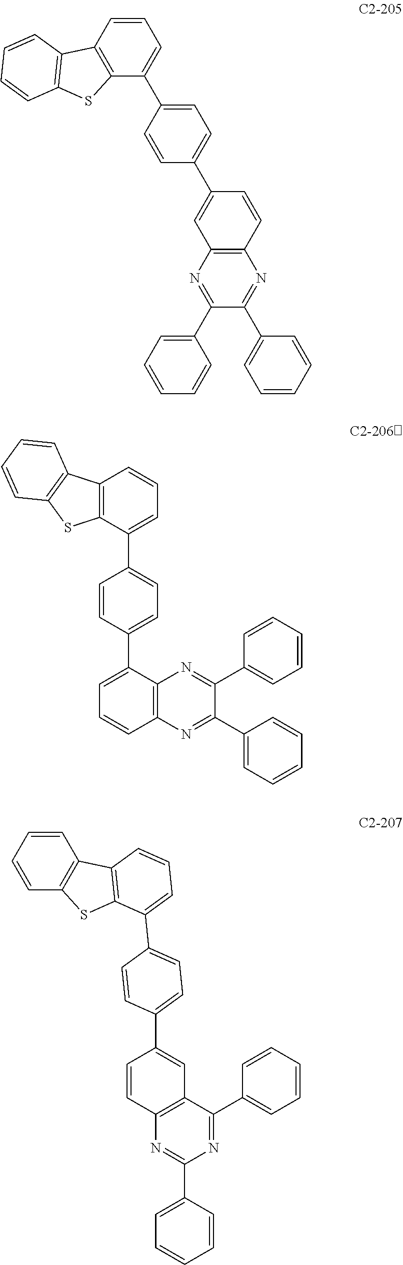 [SMILES: c1ccc(-c2nc3ccc(-c4ccc(-c5cccc6c5sc5ccccc56)cc4)cc3nc2-c2ccccc2)cc1 ; c1ccc(-c2nc3cccc(-c4ccc(-c5cccc6c5sc5ccccc56)cc4)c3nc2-c2ccccc2)cc1 ; c1ccc(-c2nc(-c3ccccc3)c3cc(-c4ccc(-c5cccc6c5sc5ccccc56)cc4)ccc3n2)cc1]

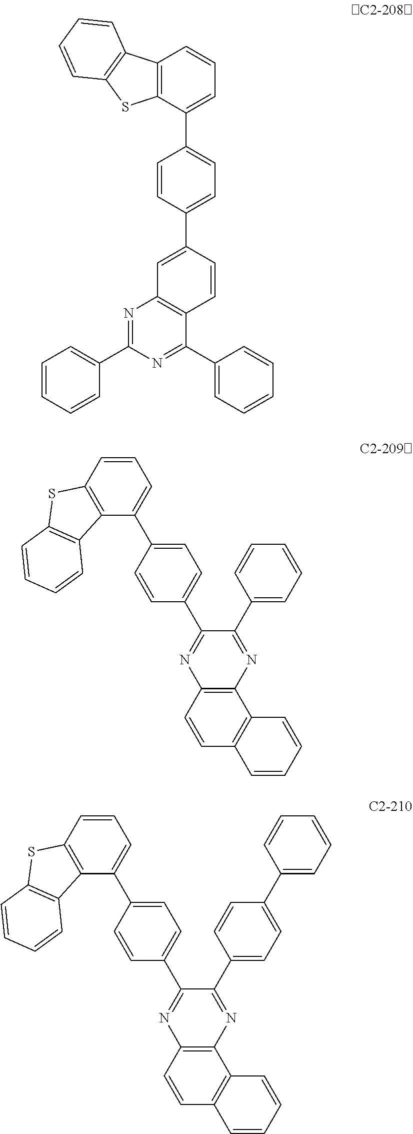

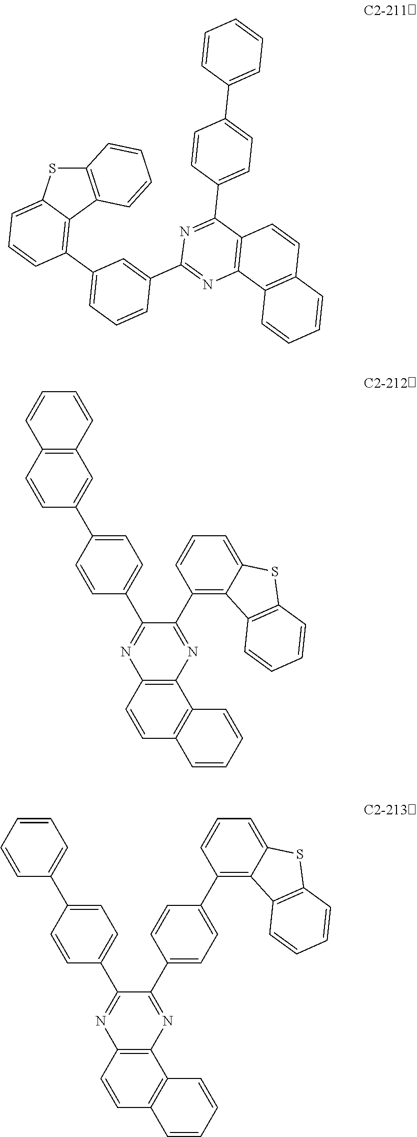 [SMILES: c1ccc(-c2ccc(-c3nc(-c4cccc(-c5cccc6sc7ccccc7c56)c4)nc4c3ccc3ccccc34)cc2)cc1 ; c1ccc2cc(-c3ccc(-c4nc5ccc6ccccc6c5nc4-c4cccc5sc6ccccc6c45)cc3)ccc2c1 ; c1ccc(-c2ccc(-c3nc4ccc5ccccc5c4nc3-c3ccc(-c4cccc5sc6ccccc6c45)cc3)cc2)cc1]

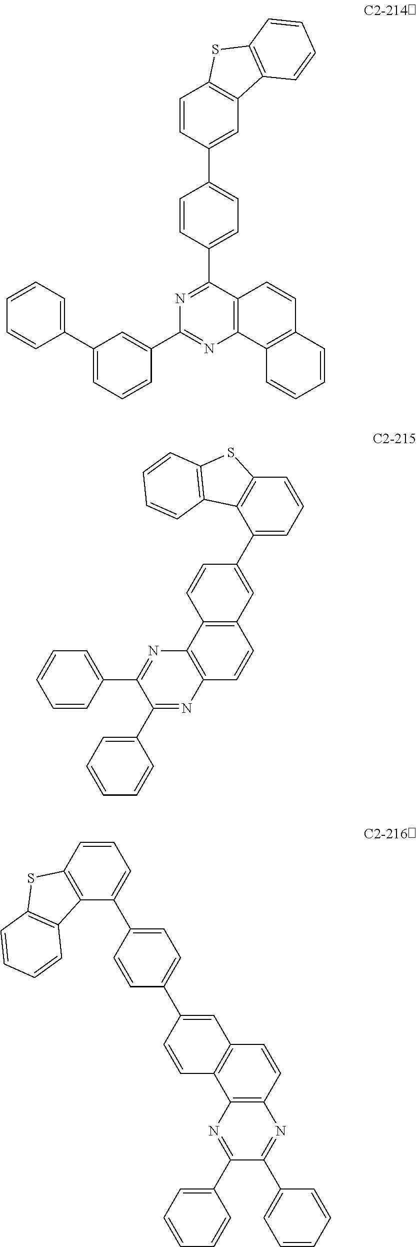 [SMILES: c1ccc(-c2cccc(-c3nc(-c4ccc(-c5ccc6sc7ccccc7c6c5)cc4)c4ccc5ccccc5c4n3)c2)cc1 ; c1ccc(-c2nc3ccc4cc(-c5cccc6sc7ccccc7c56)ccc4c3nc2-c2ccccc2)cc1 ; c1ccc(-c2nc3ccc4cc(-c5ccc(-c6cccc7sc8ccccc8c67)cc5)ccc4c3nc2-c2ccccc2)cc1]

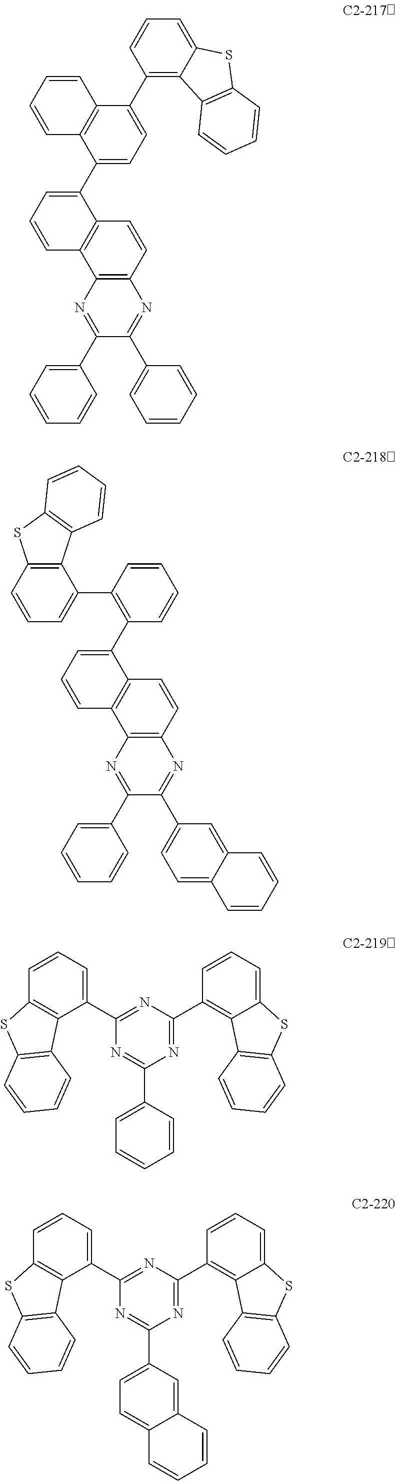

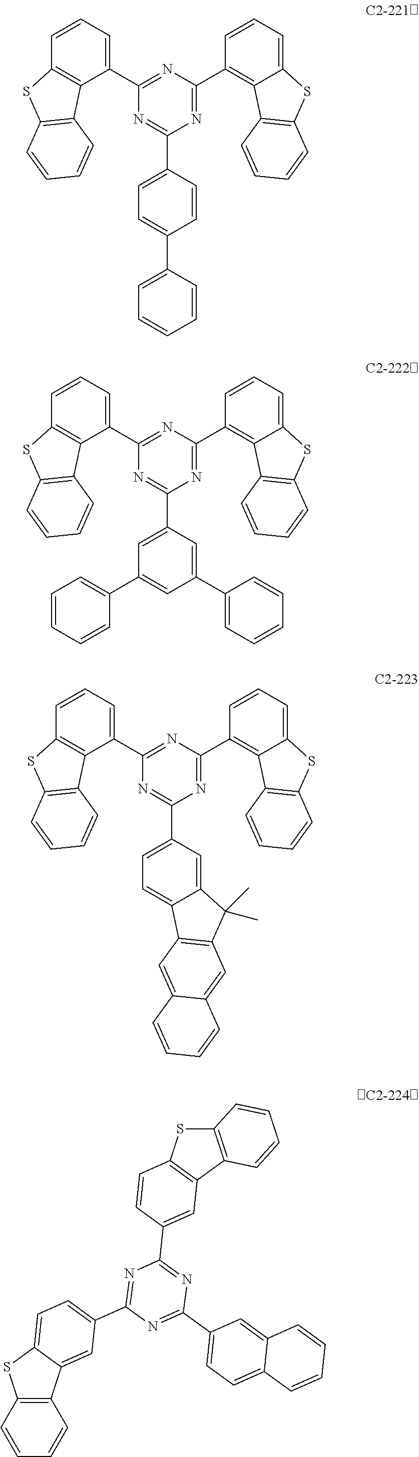

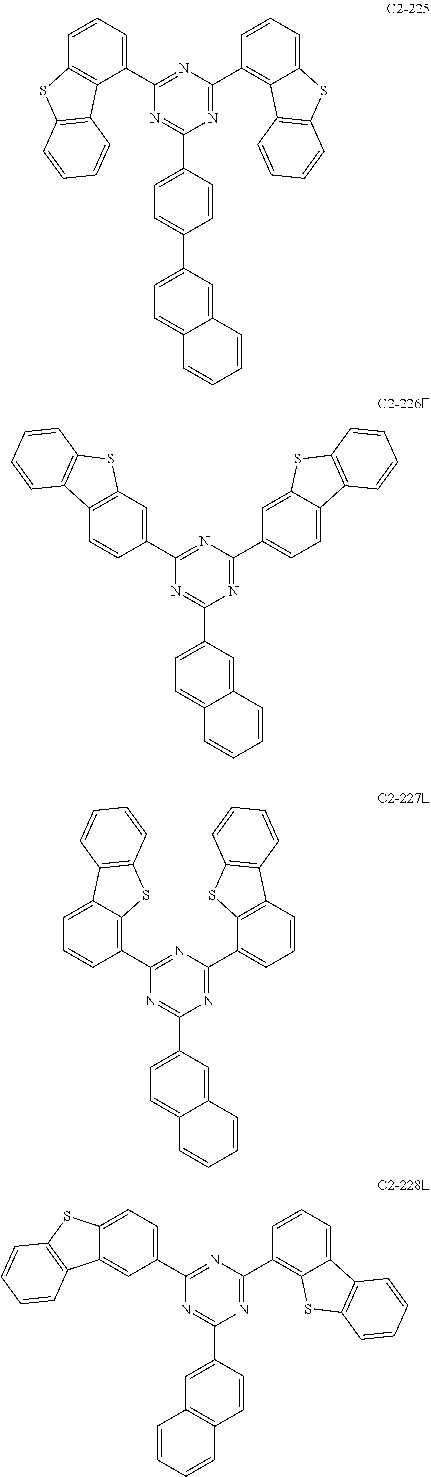 [SMILES: c1ccc2cc(-c3ccc(-c4nc(-c5cccc6sc7ccccc7c56)nc(-c5cccc6sc7ccccc7c56)n4)cc3)ccc2c1 ; c1ccc2cc(-c3nc(-c4ccc5c(c4)sc4ccccc45)nc(-c4ccc5c(c4)sc4ccccc45)n3)ccc2c1 ; c1ccc2cc(-c3nc(-c4cccc5c4sc4ccccc45)nc(-c4cccc5c4sc4ccccc45)n3)ccc2c1 ; c1ccc2cc(-c3nc(-c4ccc5sc6ccccc6c5c4)nc(-c4cccc5c4sc4ccccc45)n3)ccc2c1]

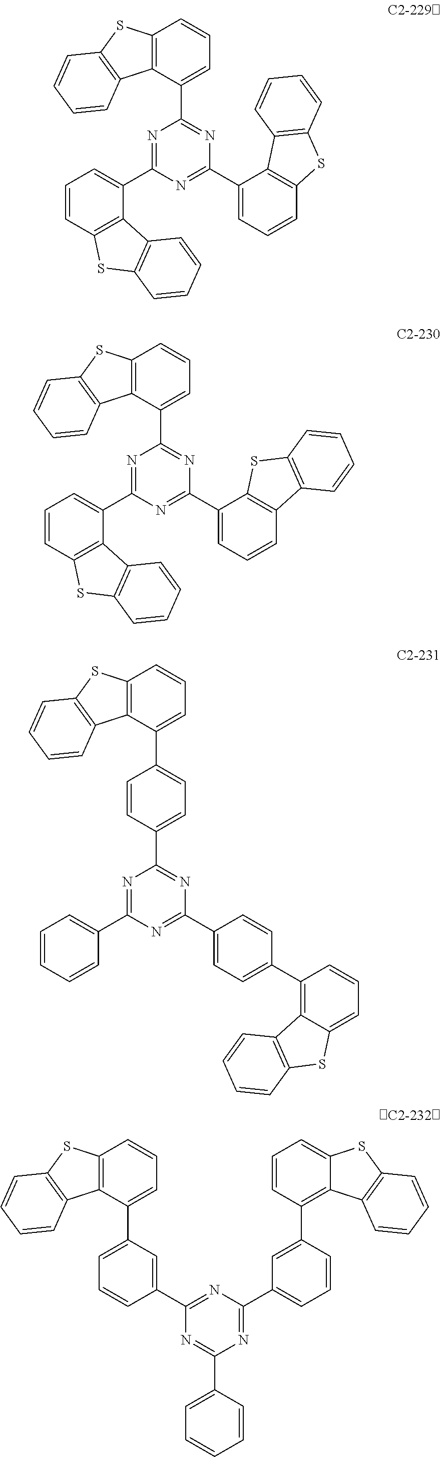

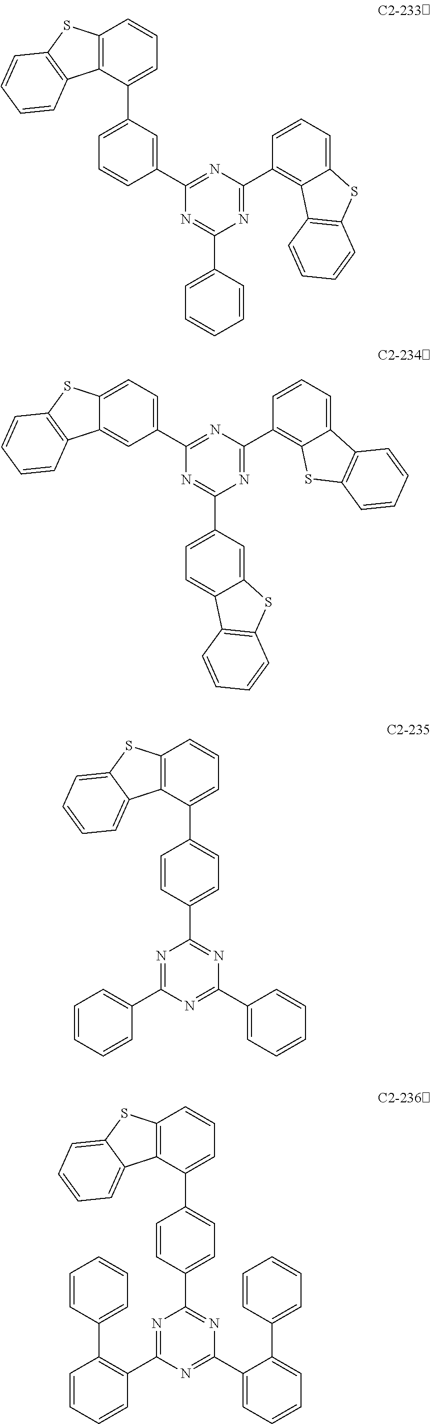 [SMILES: c1ccc(-c2nc(-c3cccc(-c4cccc5sc6ccccc6c45)c3)nc(-c3cccc4sc5ccccc5c34)n2)cc1 ; c1ccc2c(c1)sc1cc(-c3nc(-c4ccc5sc6ccccc6c5c4)nc(-c4cccc5c4sc4ccccc45)n3)ccc12 ; c1ccc(-c2nc(-c3ccccc3)nc(-c3ccc(-c4cccc5sc6ccccc6c45)cc3)n2)cc1 ; c1ccc(-c2ccccc2-c2nc(-c3ccc(-c4cccc5sc6ccccc6c45)cc3)nc(-c3ccccc3-c3ccccc3)n2)cc1]

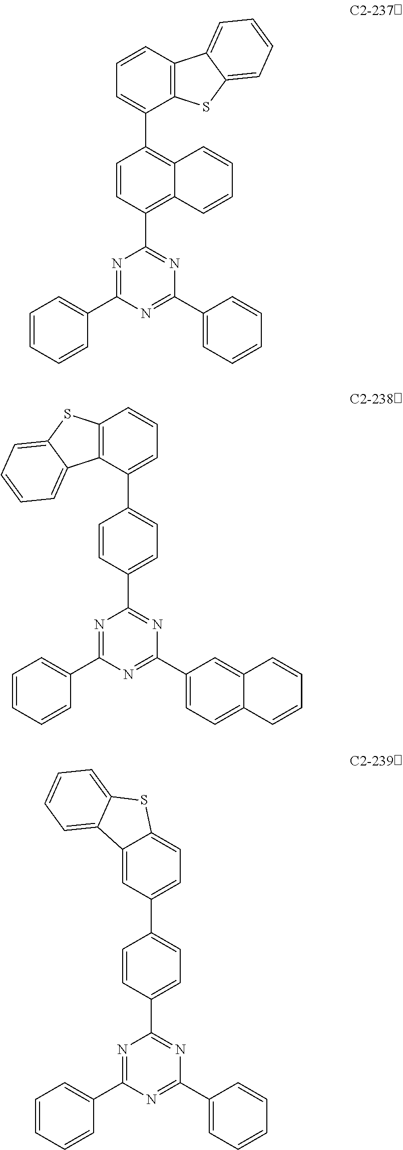 [SMILES: c1ccc(-c2nc(-c3ccccc3)nc(-c3ccc(-c4cccc5c4sc4ccccc45)c4ccccc34)n2)cc1 ; c1ccc(-c2nc(-c3ccc(-c4cccc5sc6ccccc6c45)cc3)nc(-c3ccc4ccccc4c3)n2)cc1 ; c1ccc(-c2nc(-c3ccccc3)nc(-c3ccc(-c4ccc5sc6ccccc6c5c4)cc3)n2)cc1]

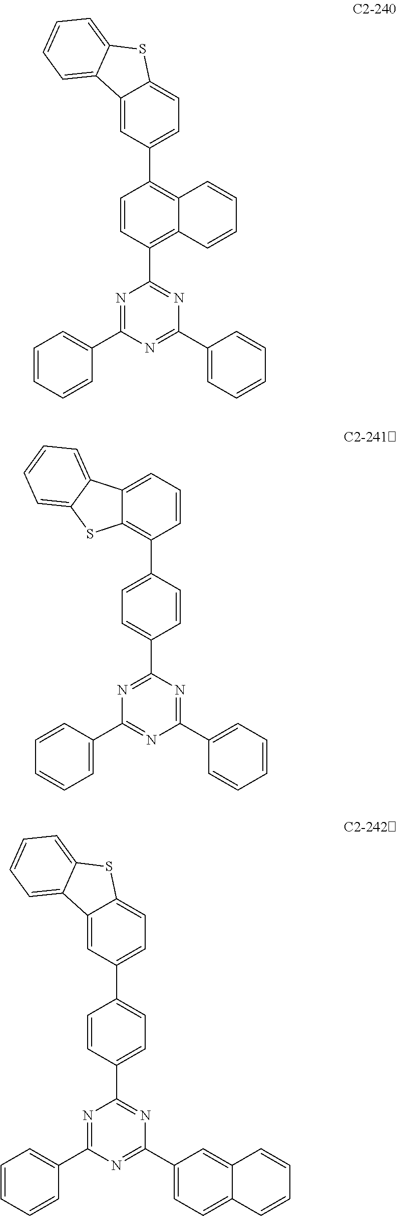

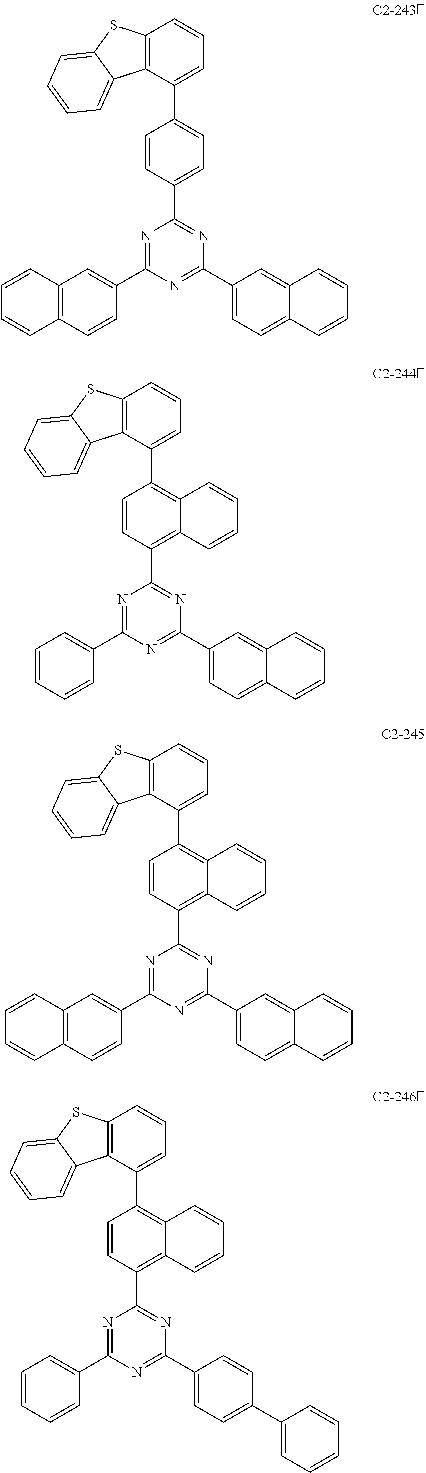 [SMILES: c1ccc2cc(-c3nc(-c4ccc(-c5cccc6sc7ccccc7c56)cc4)nc(-c4ccc5ccccc5c4)n3)ccc2c1 ; c1ccc(-c2nc(-c3ccc4ccccc4c3)nc(-c3ccc(-c4cccc5sc6ccccc6c45)c4ccccc34)n2)cc1 ; c1ccc2cc(-c3nc(-c4ccc5ccccc5c4)nc(-c4ccc(-c5cccc6sc7ccccc7c56)c5ccccc45)n3)ccc2c1 ; c1ccc(-c2ccc(-c3nc(-c4ccccc4)nc(-c4ccc(-c5cccc6sc7ccccc7c56)c5ccccc45)n3)cc2)cc1]

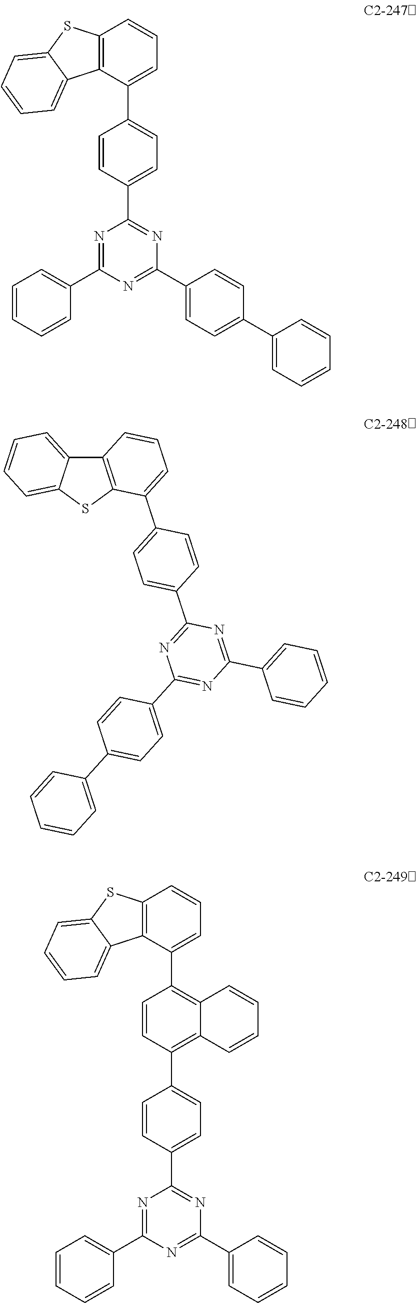 [SMILES: c1ccc(-c2ccc(-c3nc(-c4ccccc4)nc(-c4ccc(-c5cccc6sc7ccccc7c56)cc4)n3)cc2)cc1 ; c1ccc(-c2ccc(-c3nc(-c4ccccc4)nc(-c4ccc(-c5cccc6c5sc5ccccc56)cc4)n3)cc2)cc1 ; c1ccc(-c2nc(-c3ccccc3)nc(-c3ccc(-c4ccc(-c5cccc6sc7ccccc7c56)c5ccccc45)cc3)n2)cc1]

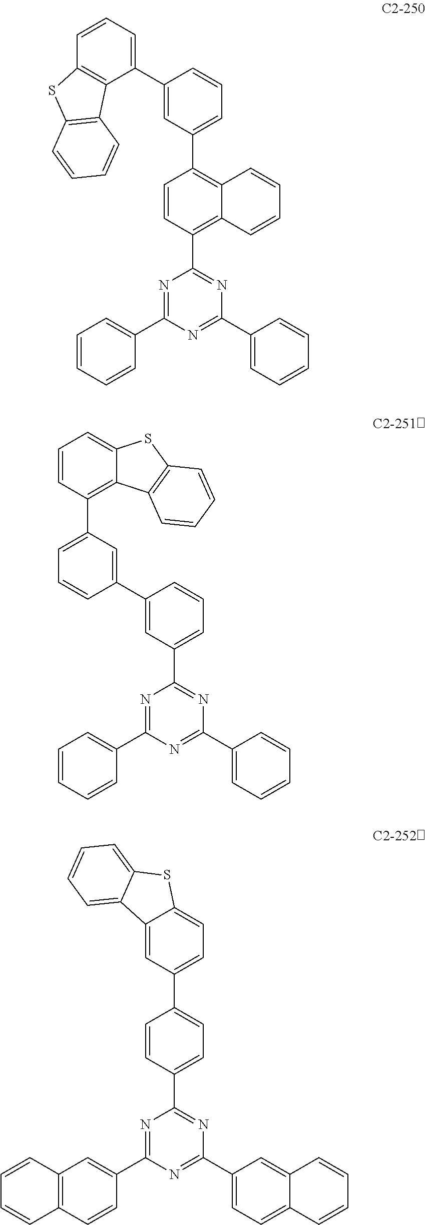 [SMILES: c1ccc(-c2nc(-c3ccccc3)nc(-c3ccc(-c4cccc(-c5cccc6sc7ccccc7c56)c4)c4ccccc34)n2)cc1 ; c1ccc(-c2nc(-c3ccccc3)nc(-c3cccc(-c4cccc(-c5cccc6sc7ccccc7c56)c4)c3)n2)cc1 ; c1ccc2cc(-c3nc(-c4ccc(-c5ccc6sc7ccccc7c6c5)cc4)nc(-c4ccc5ccccc5c4)n3)ccc2c1]

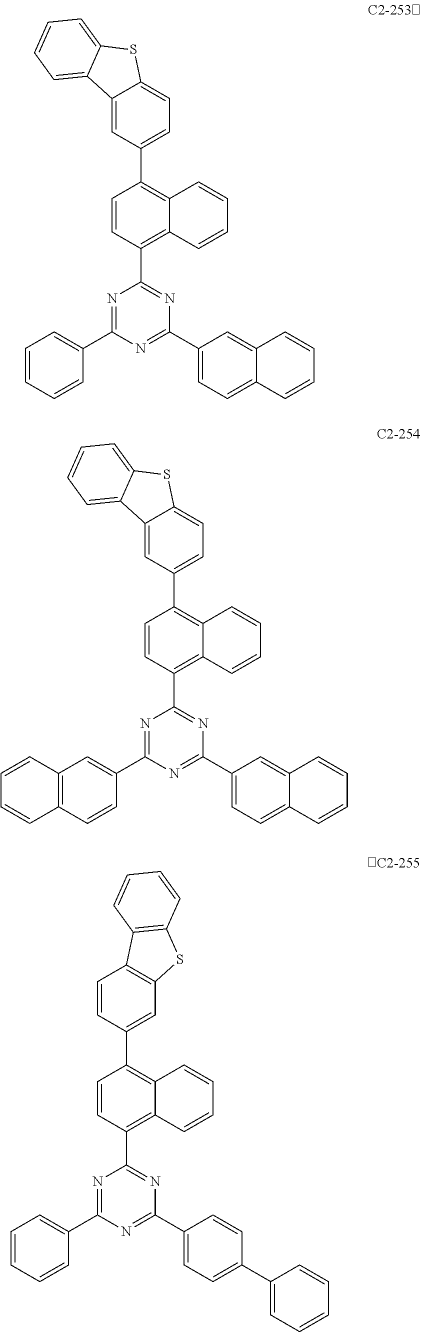 [SMILES: c1ccc(-c2nc(-c3ccc4ccccc4c3)nc(-c3ccc(-c4ccc5sc6ccccc6c5c4)c4ccccc34)n2)cc1 ; c1ccc2cc(-c3nc(-c4ccc5ccccc5c4)nc(-c4ccc(-c5ccc6sc7ccccc7c6c5)c5ccccc45)n3)ccc2c1 ; c1ccc(-c2ccc(-c3nc(-c4ccccc4)nc(-c4ccc(-c5ccc6c(c5)sc5ccccc56)c5ccccc45)n3)cc2)cc1]

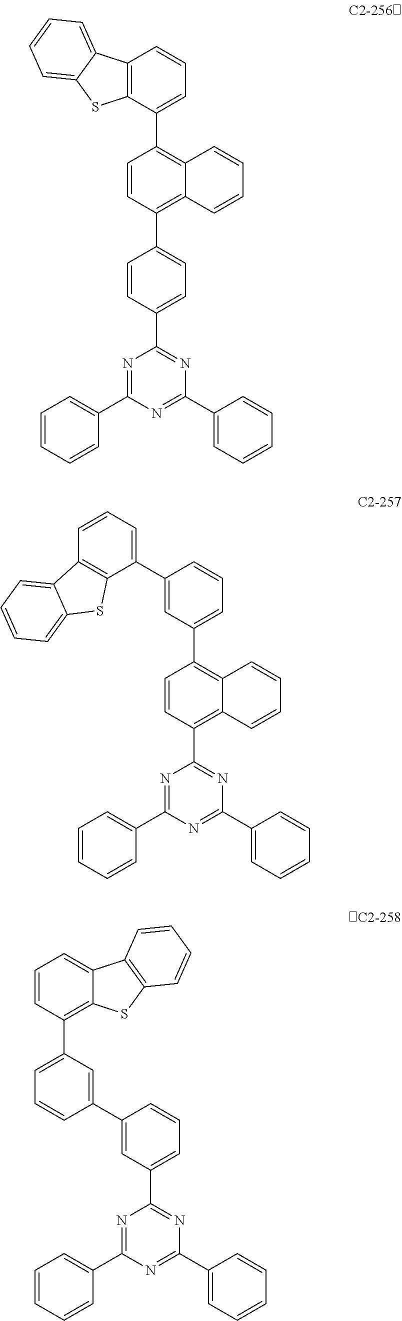 [SMILES: c1ccc(-c2nc(-c3ccccc3)nc(-c3ccc(-c4ccc(-c5cccc6c5sc5ccccc56)c5ccccc45)cc3)n2)cc1 ; c1ccc(-c2nc(-c3ccccc3)nc(-c3ccc(-c4cccc(-c5cccc6c5sc5ccccc56)c4)c4ccccc34)n2)cc1 ; c1ccc(-c2nc(-c3ccccc3)nc(-c3cccc(-c4cccc(-c5cccc6c5sc5ccccc56)c4)c3)n2)cc1]

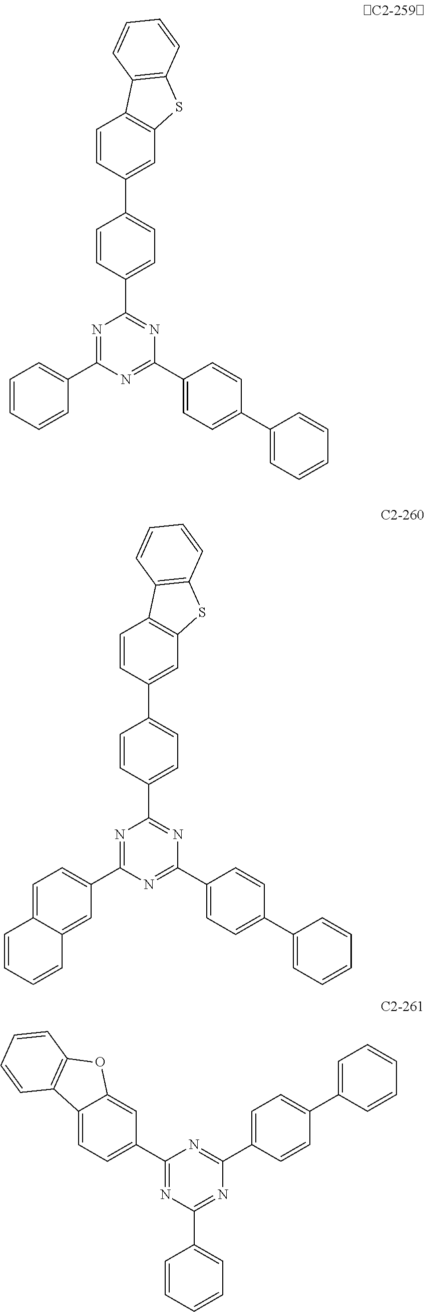 [SMILES: c1ccc(-c2ccc(-c3nc(-c4ccccc4)nc(-c4ccc(-c5ccc6c(c5)sc5ccccc56)cc4)n3)cc2)cc1 ; c1ccc(-c2ccc(-c3nc(-c4ccc(-c5ccc6c(c5)sc5ccccc56)cc4)nc(-c4ccc5ccccc5c4)n3)cc2)cc1 ; c1ccc(-c2ccc(-c3nc(-c4ccccc4)nc(-c4ccc5c(c4)oc4ccccc45)n3)cc2)cc1]

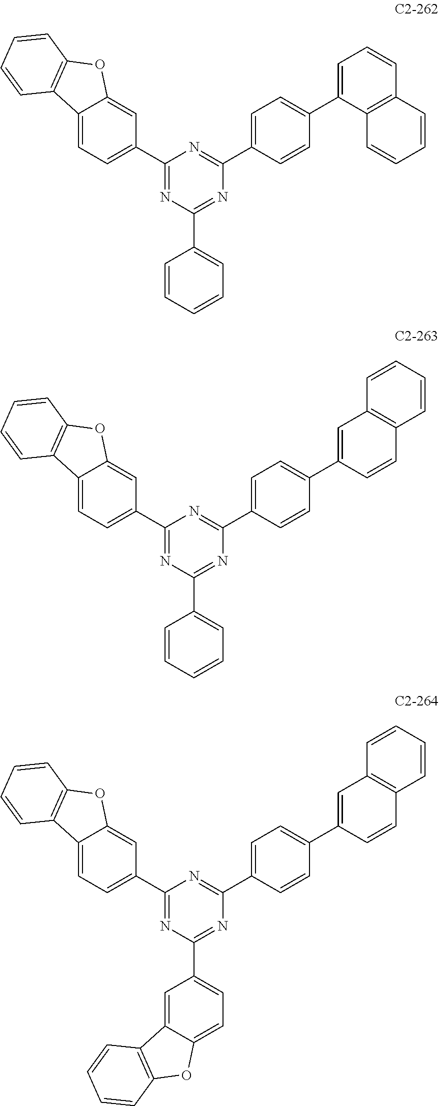 [SMILES: c1ccc(-c2nc(-c3ccc(-c4cccc5ccccc45)cc3)nc(-c3ccc4c(c3)oc3ccccc34)n2)cc1 ; c1ccc(-c2nc(-c3ccc(-c4ccc5ccccc5c4)cc3)nc(-c3ccc4c(c3)oc3ccccc34)n2)cc1 ; c1ccc2cc(-c3ccc(-c4nc(-c5ccc6c(c5)oc5ccccc56)nc(-c5ccc6oc7ccccc7c6c5)n4)cc3)ccc2c1]

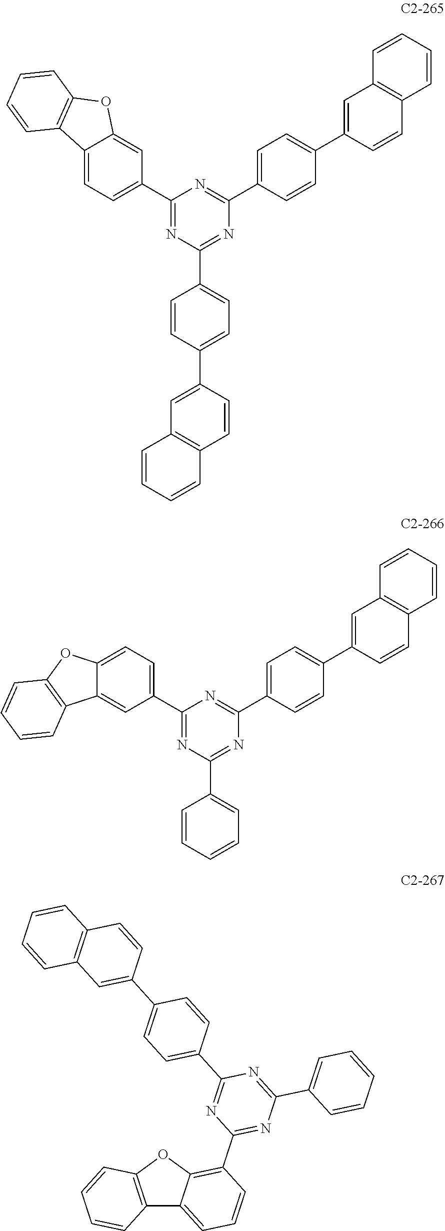 [SMILES: c1ccc2cc(-c3ccc(-c4nc(-c5ccc(-c6ccc7ccccc7c6)cc5)nc(-c5ccc6c(c5)oc5ccccc56)n4)cc3)ccc2c1 ; c1ccc(-c2nc(-c3ccc(-c4ccc5ccccc5c4)cc3)nc(-c3ccc4oc5ccccc5c4c3)n2)cc1 ; c1ccc(-c2nc(-c3ccc(-c4ccc5ccccc5c4)cc3)nc(-c3cccc4c3oc3ccccc34)n2)cc1]

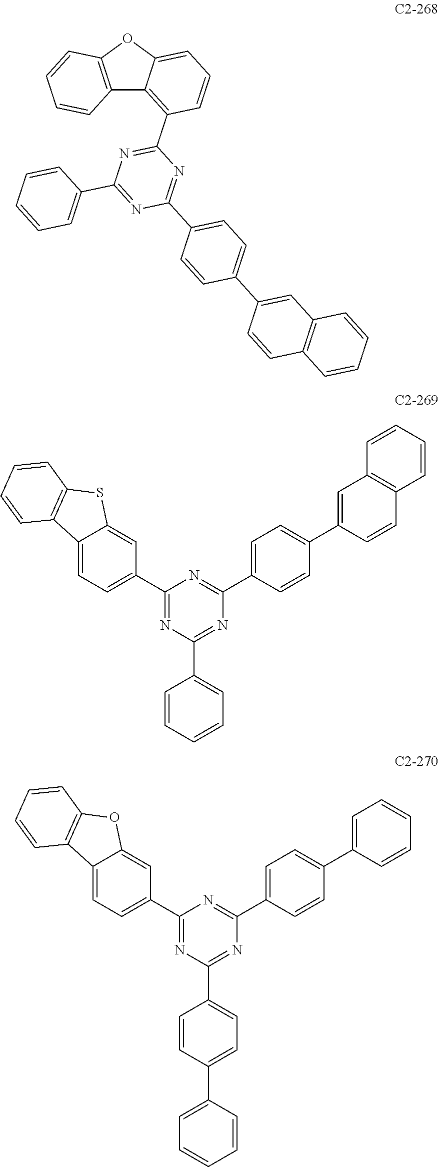 [SMILES: c1ccc(-c2nc(-c3ccc(-c4ccc5ccccc5c4)cc3)nc(-c3cccc4oc5ccccc5c34)n2)cc1 ; c1ccc(-c2nc(-c3ccc(-c4ccc5ccccc5c4)cc3)nc(-c3ccc4c(c3)sc3ccccc34)n2)cc1 ; c1ccc(-c2ccc(-c3nc(-c4ccc(-c5ccccc5)cc4)nc(-c4ccc5c(c4)oc4ccccc45)n3)cc2)cc1]

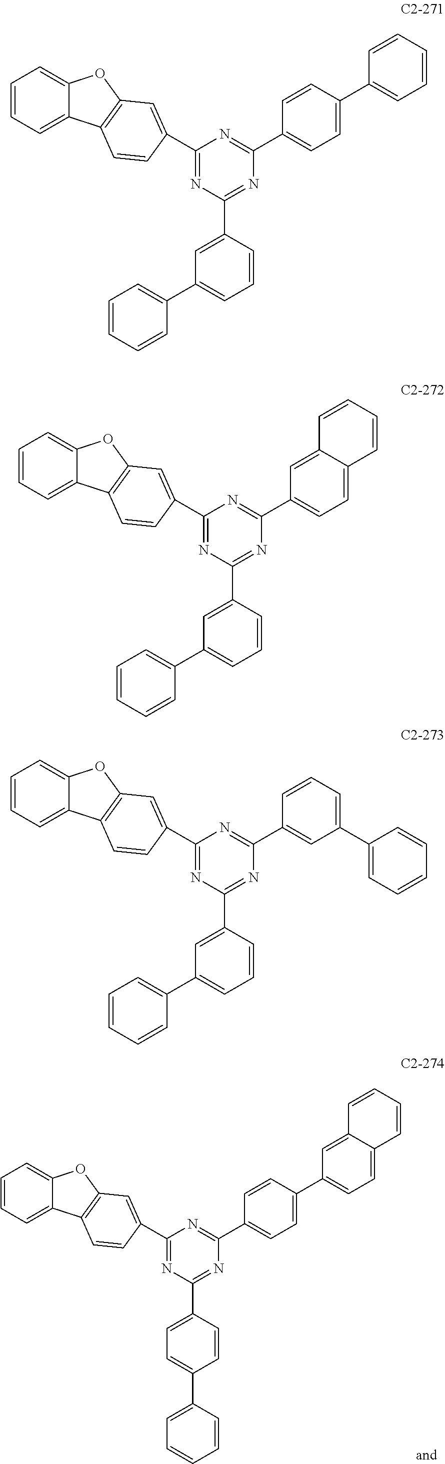 [SMILES: c1ccc(-c2ccc(-c3nc(-c4cccc(-c5ccccc5)c4)nc(-c4ccc5c(c4)oc4ccccc45)n3)cc2)cc1 ; c1ccc(-c2cccc(-c3nc(-c4ccc5ccccc5c4)nc(-c4ccc5c(c4)oc4ccccc45)n3)c2)cc1 ; c1ccc(-c2cccc(-c3nc(-c4cccc(-c5ccccc5)c4)nc(-c4ccc5c(c4)oc4ccccc45)n3)c2)cc1 ; c1ccc(-c2ccc(-c3nc(-c4ccc(-c5ccc6ccccc6c5)cc4)nc(-c4ccc5c(c4)oc4ccccc45)n3)cc2)cc1]

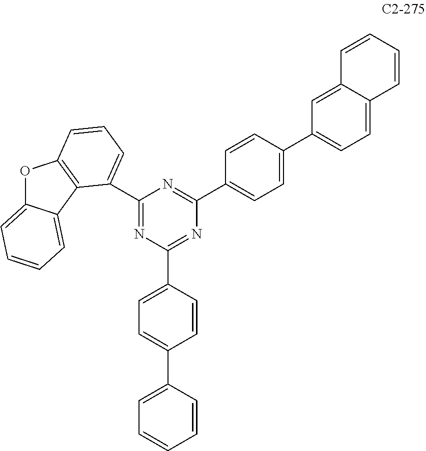 [SMILES: c1ccc(-c2ccc(-c3nc(-c4ccc(-c5ccc6ccccc6c5)cc4)nc(-c4cccc5oc6ccccc6c45)n3)cc2)cc1]

11. An organic electroluminescent device comprising an organic electroluminescent compound according to claim 1.

12. The organic electroluminescent device according to claim 11, wherein the organic electroluminescent compound is included in the light-emitting layer.

13. (canceled)
